(19) United States
(12) Patent Application Publication
(10) Pub. No.: US 2023/0271954 A1
(43) Pub. Date: Aug. 31, 2023

(54) COMPOUNDS USEFUL AS T CELL ACTIVATORS

(71) Applicant: Gossamer Bio Services, Inc., San Diego, CA (US)

(72) Inventors: Anthony Casarez, San Diego, CA (US); Terry Kellar, San Diego, CA (US)

(21) Appl. No.: 17/881,442

(22) Filed: Aug. 4, 2022

Related U.S. Application Data
(63) Continuation of application No. 17/553,563, filed on Dec. 16, 2021, now abandoned.
(60) Provisional application No. 63/126,433, filed on Dec. 16, 2020.

(51) Int. Cl. C07D 471/04; C07D 209/42; C07D 405/12; C07D 487/04

(52) U.S. Cl. CPC C07D 471/04; C07D 209/42; C07D 405/12; C07D 487/04

(57) ABSTRACT

Compounds are provided having structure (I):

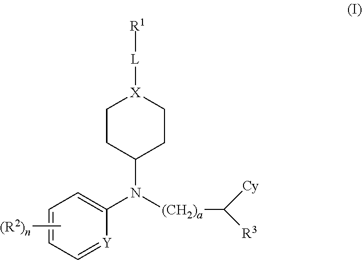

or a pharmaceutically acceptable salt, solvate, hydrate, isomer, or isotope thereof, wherein X, L, Y, R¹, R², R³ and Cy are as defined herein. Pharmaceutical compositions containing such compounds, as well as the compounds themselves, are also provided. Methods are provided wherein the compounds are inhibitors of one or both of diacylglycerol kinase alpha (DGKα) and diacylglycerol kinase zeta (DGKζ) or are useful in the treatment of diseases, disorders and conditions related to DGKα and/or DGKζ activity. More specifically, methods of treating a proliferative or a viral infection are provided by administering to a subject in need thereof, an effective amount of the pharmaceutical composition containing compounds of structure (I).

### FIELD OF THE INVENTION

The present invention generally relates to compounds that activate T cells, promote T cell proliferation, and/or exhibit antitumor activity. Provided herein are aniline compounds, compositions comprising such compounds, and methods of their use. The invention further pertains to pharmaceutical compositions comprising at least one compound according to the invention that are useful for the treatment of proliferative disorders, such as cancer, and viral infections.

### BACKGROUND

Human cancers harbor numerous genetic and epigenetic alterations, generating neoantigens potentially recognizable by the immune system (Sjoblom et al, Science, 2006, 314, 268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. However, although an endogenous immune response to cancer is observed in preclinical models and patients, this response is ineffective, and established cancers are viewed as “self” and tolerated by the immune system. Contributing to this state of tolerance, tumors may exploit several distinct mechanisms to actively subvert anti-tumor immunity. These mechanisms include dysfunctional T-cell signaling (Mizoguchi et al, Science, 1992, 258, 1795-98), suppressive regulatory cells (Facciabene et al, Cancer Res, 2012, 72, 2162-71), and the co-opting of endogenous “immune checkpoints”, which serve to down-modulate the intensity of adaptive immune responses and protect normal tissues from collateral damage, by tumors to evade immune destruction (Topalian et al, Curr. Opin. Immunol., 2012, 24, 1-6; Mellman et al, Nature, 2011, 480, 480-489).

Diacylglycerol kinases (DGKs) are lipid kinases that mediate the conversion of diacylglycerol to phosphatidic acid thereby terminating T cell functions propagated through the TCR signaling pathway. Thus, DGKs serve as intracellular checkpoints and inhibition of DGKs are expected to enhance T cell signaling pathways and T cell activation. Supporting evidence include knock-out mouse models of either DGKα or DGKζ which show a hyper-responsive T cell phenotype and improved anti-tumor immune activity (Riese et al, Journal of Biological Chemistry, 2011, 7, 5254-5265; Zha et al, Nature Immunology, 2006, 12, 1343).

Furthermore, tumor infiltrating lymphocytes isolated from human renal cell carcinoma patients were observed to overexpress DGKα which resulted in inhibited T cell function (Prinz et al, J immunology, 2012, 12, 5990-6000). Thus, DGKα and DGKζ are viewed as targets for cancer immunotherapy (Riese et al, Front Cell Dev Biol., 2016, 4, 108; Chen et al, Front Cell Dev Biol., 2016, 4, 130; Avila-Flores et al, Immunology and Cell Biology, 2017, 95, 549-563; Noessner, Front Cell Dev Biol., 2017, 5, 16; Krishna, et al, Front Immunology, 2013, 4,178; Jing, et al, Cancer Research, 2017, 77, 5676-5686.) The full length human diacylglycerol kinase alpha isoform an enzyme is disclosed as SEQ ID NO: 1, and the full length human diacylglycerol kinase zeta enzyme is disclosed as SEQ ID NO: 2.

Accordingly, there remains a need for compounds useful as inhibitors of one or both of DGKα and DGKζ. Additionally, there remains a need for compounds useful as inhibitors of one or both of DGKα and DGKζ that have selectivity over other diacylglycerol kinases, protein kinases, and/or other lipid kinases, as well as for related compositions and methods for treating diseases, disorders and conditions that would benefit from such modulation. An agent that is safe and effective in restoring T cell activation, lowering antigen threshold, enhancing anti-tumor functionality, and/or overcoming the suppressive effects of one or more endogenous immune checkpoints, such as PD-1, LAG-3 and TGFβ, would be significant for the treatment of patients with proliferative disorders, such as cancer, as well as viral infections. The present invention fulfils these and other needs as for fully provided in the following disclosure.

### BRIEF SUMMARY

Described herein are compounds that have activity as inhibitors of one or both of DGKα and DGKζ. Further, the compounds that have activity as inhibitors of one or both of DGKα and DGKζ have selectivity over other diacylglycerol kinases, protein kinases, and/or other lipid kinases. These compounds are provided to be useful as pharmaceuticals with desirable stability, bioavailability, therapeutic index, and toxicity values that are important to their druggability.

In one embodiment, a compound is provided of structure of (I):

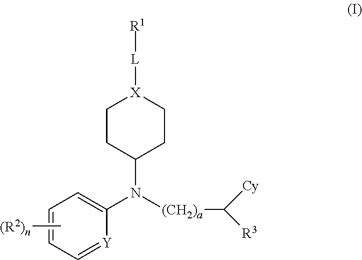

or a pharmaceutically acceptable salt, solvate, hydrate, isomer, or isotope thereof, wherein X, L, Y, R¹, R², R³ and Cy are as defined herein.

In other embodiments, compounds are provided having any of the structures (I-A), (I-B), (I-B-cis) or (I-B-trans), as defined herein, or a pharmaceutically acceptable salt, solvate, hydrate, isomer, or isotope thereof.

In some embodiments, compounds are provided having any of the structures (I-A-1), (I-A-2), (I-A-3), (I-A-4), (I-B-1), (I-B-2), (I-B-3) or (I-B-4), as defined herein, or a pharmaceutically acceptable salt, solvate, hydrate, isomer, or isotope thereof.

In yet other embodiments, pharmaceutical compositions are provided comprising a carrier or excipient and a compound having structure (I), or a pharmaceutically acceptable salt, solvate, hydrate, isomer, or isotope thereof.

In specific embodiments, pharmaceutical compositions are provided comprising substructures of structure (I) with structures (I-A), (I-B), (I-B-cis), (I-B-trans), (I-A-1), (I-A-2), (I-A-3), (I-A-4), (I-B-1), (I-B-2), (I-B-3) or (I-B-4) as defined herein or a pharmaceutically acceptable salt, solvate, hydrate, isomer or isotope thereof.

In one embodiment, methods are provided wherein the compounds are inhibitors of one or both of diacylglycerol kinase alpha (DGKα) and diacylglycerol kinase zeta (DGKζ) or are useful in the treatment of diseases, disorders and conditions related to DGKα and/or DGKζ activity.

In more specific embodiments, the diacylglycerol kinase alpha (DGKα) or diacylglycerol kinase zeta (DGKζ) dependent condition is a proliferative disorder or a viral infection.

In one embodiment, the methods of treating the diacylglycerol kinase alpha (DGKα) or diacylglycerol kinase zeta (DGKζ) dependent condition condition are provided which comprise administering an effective amount of a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) as defined herein or a pharmaceutically acceptable salt, solvate, hydrate, isomer, or isotope thereof.

In yet another embodiment, methods are provided for treating a proliferative disorder or a viral infection, comprising administering to a subject in need thereof an effective amount of the pharmaceutical composition, comprising a carrier or excipient and a compound having structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof.

In another embodiment, compounds are provided having one or more of the structures disclosed herein, or a pharmaceutically acceptable salt, solvate, hydrate, isomer, or isotope thereof.

### DETAILED DESCRIPTION

As mentioned above, compounds are provided that have activity as inhibitors of one or both of DGKα and DGKζ. Further, the compounds that have activity as inhibitors of one or both of DGKα and DGKζ and have selectivity over other diacylglycerol kinases, protein kinases, and/or other lipid kinases.

Unless defined otherwise, all technical and scientific terms used herein have the same meaning as is commonly understood by one of skill in the art to which the claimed subject matter belongs. It is to be understood that the detailed description is exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

As used herein, ranges and amounts can be expressed as “about” a particular value or range. About also includes the exact amount. Hence “about 100 μL” means “about 100 μL” and also “100 μL.” In some embodiments, about means within 5% of the value. Hence, “about 100 μL” means 95-105 μL. In some embodiments, about means within 4% of the value. In some embodiments, about means within 3% of the value. In some embodiments, about means within 2% of the value. In some embodiments, about means within 1% of the value. Generally, the term “about” includes an amount that would be expected to be within experimental error.

### Definitions

“Alkyl” means a straight chain or branched saturated hydrocarbon group. “Lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 2 carbon atoms. Examples of straight chain lower alkyl groups include, but are not limited to, methyl, ethyl, n-propyl, n-butyl, n-pentyl, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

“Alkenyl” groups include straight and branched chain alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to —CH═CH₂, —CH═CH(CH₃), —CH═C(CH₃)₂, —C(CH₃)═CH₂, —C(CH₃)═CH(CH₃), —C(CH₂CH₃)═CH₂, —CH═CHCH₂CH₃, —CH═CH(CH₂)₂CH₃, —CH═CH(CH₂)₃CH₃, —CH═CH(CH₂)₄CH₃, or vinyl.

“Alkynyl” groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

As used herein, “alkylene” means a divalent alkyl group. Examples of straight chain lower alkylene groups include, but are not limited to, methylene (i.e., —CH₂—), ethylene (i.e., —CH₂CH₂—), propylene (i.e., —CH₂CH₂CH₂—), and butylene (i.e., —CH₂CH₂CH₂CH₂—). As used herein, “heteroalkylene” is an alkylene group of which one or more carbon atoms is replaced with a heteroatom such as, but not limited to, N, O, S, or P.

“Alkoxy” refers to an alkyl as defined above joined by way of an oxygen atom (i.e., —O-alkyl). Examples of lower alkoxy groups include, but are not limited to, methoxy, ethoxy, n-propoxy, n-butoxy, isopropoxy, sec-butoxy, tert-butoxy, and the like.

“Carbocycle” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity.

In one embodiment, carbocycle includes cycloalkyl as defined above.

In another embodiment, carbocycle includes aryl as defined above.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

“Aryl” groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like).

“Carbocyclealkyl” refers to an alkyl as defined above with one or more hydrogen atoms replaced with carbocycle. Examples of carbocyclealkyl groups include, but are not limited to, benzyl and the like.

As used herein, “heterocycle” or “heterocyclyl” groups include aromatic and non-aromatic ring compounds (heterocyclic rings) containing 3 or more ring members, of which one or more is a heteroatom such as, but not limited to, N, O, S, or P. A heterocycle group as defined herein can be a heteroaryl group or a partially or completely saturated cyclic group including at least one ring heteroatom. In some embodiments, heterocycle groups include 3 to 20 ring members, whereas other such groups have 3 to 15 ring members. At least one ring contains a heteroatom, but every ring in a polycyclic system need not contain a heteroatom. For example, a dioxolanyl ring and a benzodioxolanyl ring system (methylenedioxyphenyl ring system) are both heterocycle groups within the meaning herein. A heterocycle group designated as a C₂-heterocycle can be a 5-membered ring with two carbon atoms and three heteroatoms, a 6-membered ring with two carbon atoms and four heteroatoms and so forth. Likewise, a C₄-heterocycle can be a 5-membered ring with one heteroatom, a 6-membered ring with two heteroatoms, and so forth. The number of carbon atoms plus the number of heteroatoms sums up to equal the total number of ring atoms. A saturated heterocyclic ring refers to a heterocyclic ring containing no unsaturated carbon atoms.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

In one embodiment, heterocyclyl includes heteroaryl.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“Heterocyclealkyl” refers to an alkyl as defined above with one or more hydrogen atoms replaced with heterocycle. Examples of heterocyclealkyl groups include, but are not limited to, morpholinoethyl and the like.

“Halo” or “halogen” refers to fluorine, chlorine, bromine and iodine.

“Hydroxy” refers to —OH.

“Haloalkyl” refers to an alkyl as defined above with one or more hydrogen atoms replaced with halogen. Examples of lower haloalkyl groups include, but are not limited to, —CF₃, —CH₂CF₃, and the like.

“Haloalkoxy” refers to an alkoxy as defined above with one or more hydrogen atoms replaced with halogen. Examples of lower haloalkoxy groups include, but are not limited to —OCF₃, —OCH₂CF₃, and the like.

“Hydroxyalkyl” refers to an alkyl as defined above with one or more hydrogen atoms replaced with —OH. Examples of lower hydroxyalkyl groups include, but are not limited to —CH₂OH, —CH₂CH₂OH, and the like.

As used herein, the term “optionally substituted” refers to a group (e.g., an alkyl, carbocycle, or heterocycle) having 0, 1, or more substituents, such as 0-25, 0-20, 0-10 or 0-5 substituents. Substituents include, but are not limited to —OR^{a}, —NR^{a}R^{b}, —S(O)₂R^{a} or —S(O)₂OR^{a}, halogen, cyano, alkyl, haloalkyl, alkoxy, carbocycle, heterocycle, carbocyclalkyl, or heterocyclealkyl, wherein each R^{a} and R^{b} is, independently, H, alkyl, haloalkyl, carbocycle, or heterocycle, or R^{a} and R^{b}, together with the atom to which they are attached, form a 3-8 membered carbocycle or heterocycle.

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized to be substantially free of their enantiomeric or diastereomeric partners, and these are all within the scope of certain embodiments of the disclosure. The isomers resulting from the presence of a chiral center comprise a pair of non-superimposable isomers that are called “enantiomers.” Single enantiomers of a pure compound are optically active (i.e., they can rotate the plane of plane polarized light and designated R or S). The term also encompasses isomers arising from substitution patterns across double bonds, in particular (E)- and (Z)-isomers, or cis- and trans-isomers. E-Z configuration describes the absolute stereochemistry across double bonds having two, three or four substituents. Following the Cahn-Ingold-Prelog priority rules (CIP rules), each substituent on a double bond is assigned a priority, and the positions of the higher of the two substituents on each carbon determined. If the two groups of higher priority are on the same side of the double bond (cis to each other), the bond is assigned Z (“zusammen”, German for “together”). If the two groups of higher priority are on opposite sides of the double bond (trans to each other), the bond is assigned E (“entgegen”, German for “opposite”).

1,4-disubstituted cyclohexanes, as described herein, may exist as cis and trans isomers. Each isomer may be isolated separately or exist as mixtures. The mixtures may be predominantly one isomer, e.g. 99.9%, or 99% or 90%, predominantly the other isomer, enriched in one or the other of the isomer (e.g. an 80/20 mixture, or a 40/60 mixture), or be approximately equal mixtures. Assignment of cis or trans is illustrated in the figure below.

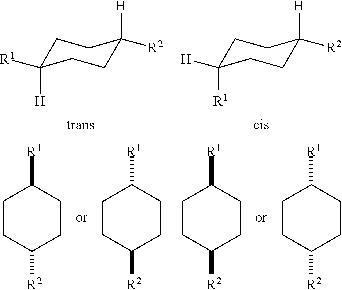

“Isolated optical isomer” means a compound which has been substantially purified from the corresponding optical isomer(s) of the same formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has several isotopes, fluorine 19 is longest-lived. Thus, an isotope of a compound having the structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, -galactaric, and galacturonic acid.

Although pharmaceutically unacceptable salts are not generally useful as medicaments, such salts may be useful, for example as intermediates in the synthesis of the compounds described herein, for example in their purification by recrystallization.

In some embodiments, the compounds are pharmaceutically acceptable salts. In some embodiments, the compounds are isomers. In some embodiments, the compounds are racemates. In some embodiments, the compounds are solvates. In some embodiments, the compounds are hydrates. In some embodiments, the compounds are isotopes. In some embodiments, the compounds are tautomers.

A “tautomer” refers to a proton shift from one atom of molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements, where tautomerization is possible, a chemical equilibrium of the tautomers will exist.

In one embodiment, compounds of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) may include tautomers.

### Compounds

As detailed above, the present disclosure provides compounds compound having activity as inhibitors of one or both of DGKα and DGKζ. Accordingly, one embodiment provides a compound having the following structure (I):

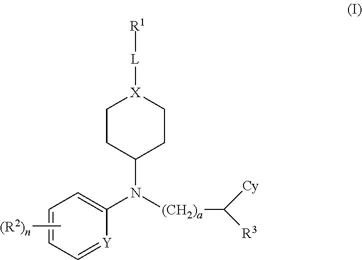

or a pharmaceutically acceptable salt, solvate, hydrate, isomer, or isotope thereof, wherein:

R¹ is an 8-13 membered heteroaryl comprising 1-4 ring nitrogen atoms and substituted with 1, 2, 3 or 4 substituents, wherein each substituent is independently OH, oxo, halo, CN, NO₂, C₁₋₄ alkyl, O—C₁₋₄ alkyl, C₁₋₄ alkenyl or C₃₋₆ cycloalkyl;

X is N or CH;

when X is N, then L is a bond, CH₂, C(O) or CHMe, and

when X is CH, then L is a bond, NH, NMe or NHC(O);

Y is N or CH;

each occurrence of R² is independently halo, OH or OMe;

a is 0, 1 or 2;

n is 0, 1, 2, 3 or 4;

R³ is H or C₁₋₄ alkyl; and

Cy is a 3-6 membered cycloalkyl or heterocycloalkyl comprising 0 or 1 ring oxygen atom.

In another embodiment, are provided compounds of the following structures (I-A), (I-B), (I-B-cis) or (I-B-trans):

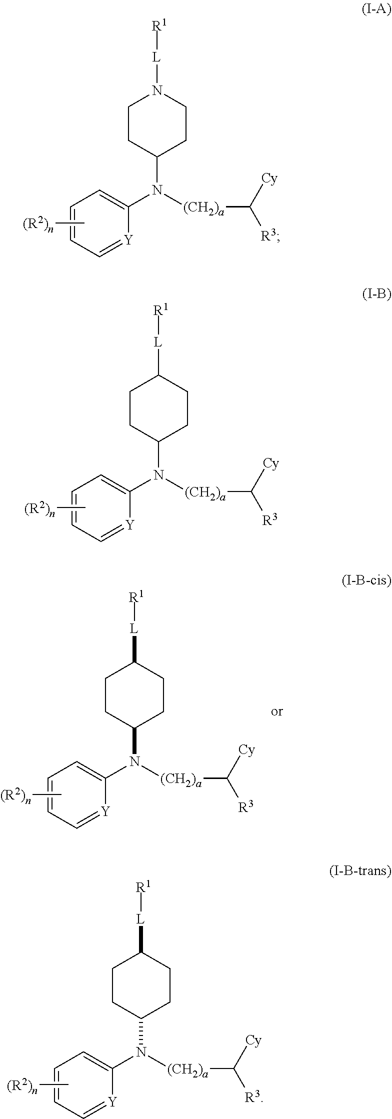

or a pharmaceutically acceptable salt, solvate, hydrate, isomer, or isotope thereof.

In yet another embodiment, a compound is provided having structure (I-A):

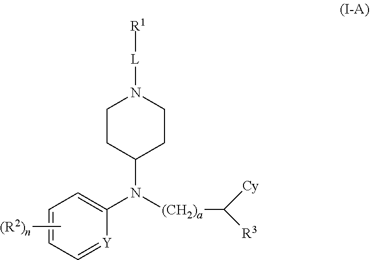

or a pharmaceutically acceptable salt, solvate, hydrate, isomer, or isotope thereof.

In some embodiments, a compound is provided having structure (I-B):

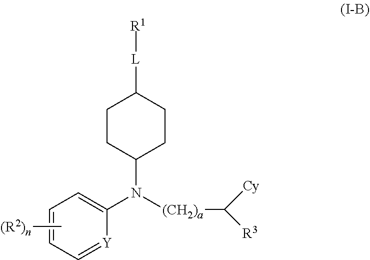

or a pharmaceutically acceptable salt, solvate, hydrate, isomer, or isotope thereof.

In one embodiment, a compound is provided having structure (I-B-cis):

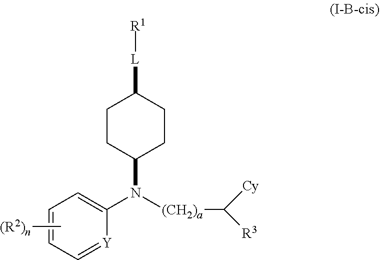

or a pharmaceutically acceptable salt, solvate, hydrate, isomer, or isotope thereof.

In yet another embodiment, a compound is provided having structure (I-B-trans):

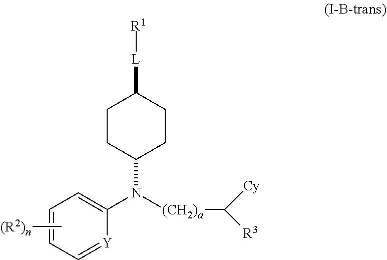

or a pharmaceutically acceptable salt, solvate, hydrate, isomer, or isotope thereof.

In some embodiments, an isomer is provided wherein the isomer with structure (I-B-cis) or (I-B-trans) are present as mixtures. In other embodiments, an isomer is provided wherein the mixture is predominantly one isomer, i.e., 99.9% or 99% or at least 90% of (I-B-cis) isomer. In other embodiments, an isomer is provided wherein the mixture is 99.9% or 99% or at least 90% of (I-B-trans) isomer.

In yet other embodiments, the mixture is enriched in either the (I-B-cis) or the (I-B-trans) isomer. In some embodiments, the isomer is an 80/20 mixture of one isomer over the other. In yet other embodiments, the isomer is a 60/40 mixture of one isomer over the other. In some embodiments, the isomer is approximately an equal mixture of one isomer over the other. In yet other embodiments, the (I-B-cis) and (I-B-trans) are present as 50/50 mixtures.

In yet another embodiment, compounds are provided having one of the following structures (I-A-1), (I-A-2), (I-A-3), (I-A-4), (I-B-1), (I-B-2), (I-B-3) or (I-B-4):

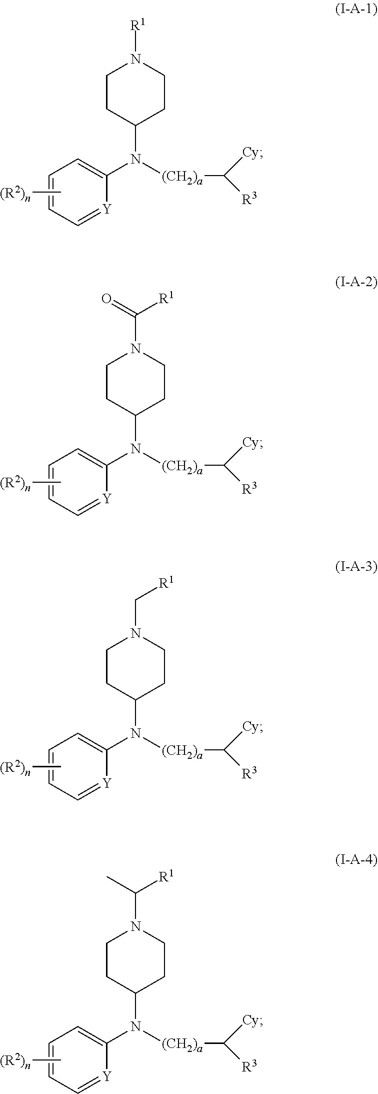

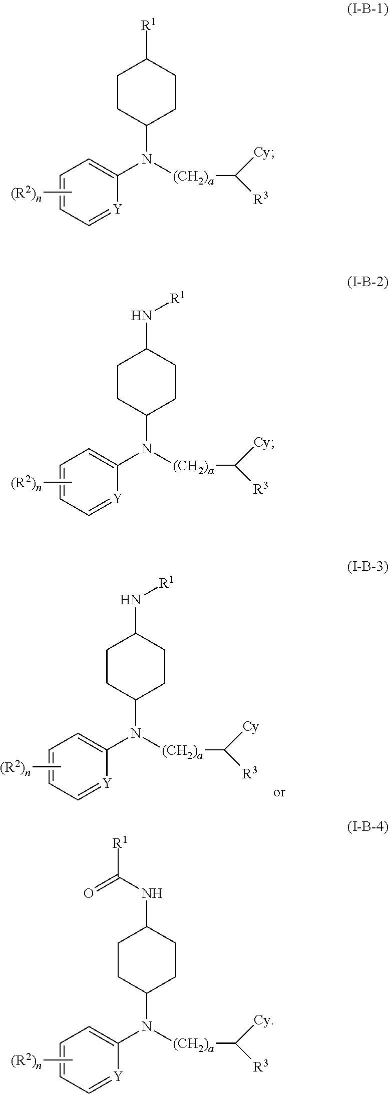

or a pharmaceutically acceptable salt, solvate, hydrate, isomer, or isotope thereof.

In one embodiment, R¹ is a 9-, 10- or 13-membered heteroaryl. In some embodiments, R¹ is a 9-membered heteroaryl. In other embodiments, R¹ is a 10-membered heteroaryl. Yet in another embodiment, R¹ is a 13-membered heteroaryl.

In one embodiment, the heteroaryl comprises 1 to 3 ring nitrogen atoms. In yet another embodiment, the heteroaryl comprises one ring nitrogen atom. In some other embodiments, the heteroaryl comprises 2 ring nitrogen atoms. In one embodiment, the heteroaryl comprises 3 ring nitrogen atoms.

In another embodiment, R¹ is a 13-membered heteroaryl wherein the heteroaryl ring comprises 2, 3 or 4 ring nitrogen atoms.

In one embodiment, R¹ is substituted with 1 substituent, wherein the substituent is OH, halogen, CN, NO₂, C₁₋₄ alkyl, O—C₁₋₄ alkyl, C₁₋₄ alkenyl or C₃₋₆ cycloalkyl. In some other embodiments, R¹ is substituted with 2 substituents, wherein each substituent is independently OH, halo, CN, NO₂, C₁₋₄ alkyl, O—C₁₋₄ alkyl, C₁₋₄ alkenyl or C₃₋₆ cycloalkyl. In yet other embodiments, R¹ is substituted with 3 substituents, wherein each substituent is independently oxo, halogen, CN, NO₂ or C₁₋₄ alkyl. In some embodiments, R¹ is substituted with 4 substituents, wherein each substituent is independently oxo, halogen, CN, NO₂ or C₁₋₄ alkyl.

In another embodiment, R¹ is:

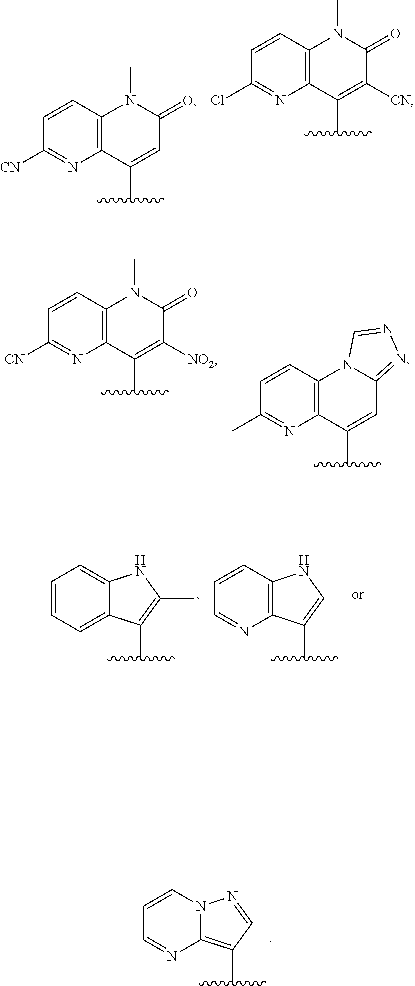

In one embodiment

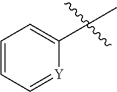

is unsubstituted.

In another embodiment, n in —(R²)ₙ is 1, 2, 3 or 4 and wherein each occurrence of R² is independently halo, OH or OMe. In some other embodiments, n is 1 and wherein R² is OH. In yet other embodiments, n is 1 and wherein R² is OMe. In one embodiment, n is 1 or 2 and wherein R² is halo. In one embodiment, halo is fluoro.

In one embodiment, n is 2 and wherein each R² is independently OH and halo. In another embodiment, halo is fluoro.

In yet another embodiment, n is 2 and wherein each R² is independently OMe and halo.

In one embodiment, Y is CH.

In another embodiment, Y is N.

In another embodiment,

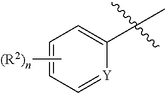

has one of the following structures:

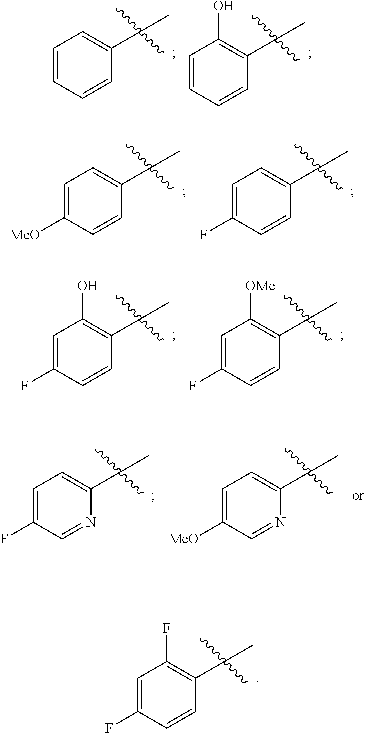

In one embodiment, a is 0 or 1.

In another embodiment, R³ is H or methyl. In yet another embodiment, R³ is H. In yet another embodiment, R³ is methyl.

In some embodiments, a is 0 or 1 and wherein R³ is H.

In one embodiment, Cy is cyclopropyl or a 4- or 5-membered heterocycloalkyl comprising 1 oxygen atom. In another embodiment, Cy is a cyclopropyl. In yet another embodiment, Cy is a 4- or 5-membered heterocycloalkyl comprising 1 oxygen atom. In one embodiment, Cy is oxetane.

In one embodiment, Cy is cyclopropyl and wherein R³ is H.

In another embodiment, Cy is 4- or 5-membered heterocycloalkyl comprising 1 oxygen atom and wherein R³ is H.

In one embodiment, Cy is cyclopropyl and wherein a is 0.

In another embodiment, Cy is cyclopropyl and wherein a is 1.

In some embodiments, Cy is 4- or 5-membered heterocycloalkyl comprising 1 oxygen atom and wherein a is 0.

In yet another embodiment, Cy is 4- or 5-membered heterocycloalkyl comprising 1 oxygen atom and wherein a is 1.

In some embodiments, Cy is cyclopropyl or oxetane, wherein a is 0 or 1, and wherein R³ is H.

Representative compounds of structure (I) as well as structures (I-A), (I-B), (I-B-cis), (I-B-trans), (I-A-1), (I-A-2), (I-A-3), (I-A-4), (I-B-1), (I-B-2), (I-B-3) or (I-B-4) as applicable, include, but not limited to, any one of the compounds listed in Table 1 below, or pharmaceutically acceptable salt, solvate, hydrate, isomer, or isotope thereof.

To this end, representative compounds are identified herein by their respective “Compound Number”, or “Example Number” which is sometimes abbreviated as “Compound No.”, “Cmpd. No.”, “No.”, “Example No.”, “Eg. No.”, or “Ex”, and the like.

TABLE 1
Compounds of structure (I)

| CMPD. NO. | STRUCTURE | NAME |
|---|---|---|
| 1A | 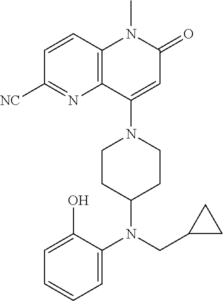 [SMILES: Cn1c(=O)cc(N2CCC(N(CC3CC3)c3ccccc3O)CC2)c2nc(C#N)ccc21] | 8-(4-((cyclopropylmethyl)(2-hydroxyphenyl)amino) piperidin-1-yl)-5-methyl-6-oxo-5,6-dihydro-1,5- naphthyridine-2-carbonitrile |
| 1B | 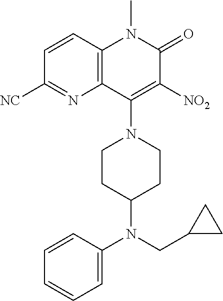 [SMILES: Cn1c(=O)c([N+](=O)[O-])c(N2CCC(N(CC3CC3)c3ccccc3)CC2)c2nc(C#N)ccc21] | 8-(4-((cyclopropylmethyl)(phenyl)amino)piperidin-1- yl)-5-methyl-7-nitro-6-oxo-5,6-dihydro-1,5- naphthyridine-2-carbonitrile |
| 1C | 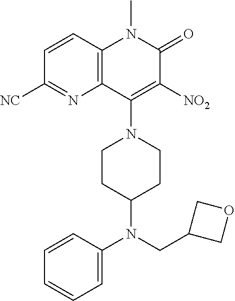 [SMILES: Cn1c(=O)c([N+](=O)[O-])c(N2CCC(N(CC3COC3)c3ccccc3)CC2)c2nc(C#N)ccc21] | 5-methyl-7-nitro-8-(4-((oxetan-3-ylmethyl)(phenyl) amino)piperidin-1-yl)-6-oxo-5,6-dihydro-1,5- naphthyridine-2-carbonitrile |
| 1D | 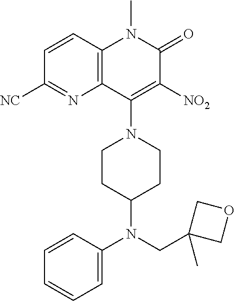 [SMILES: Cn1c(=O)c([N+](=O)[O-])c(N2CCC(N(CC3(C)COC3)c3ccccc3)CC2)c2nc(C#N)ccc21] | 5-methyl-8-(4-(((3-methyloxetan-3-yl)methyl)(phenyl) amino)piperidin-1-yl)-7-nitro-6-oxo-5,6-dihydro-1,5- naphthyridine-2-carbonitrile |
| 1E | 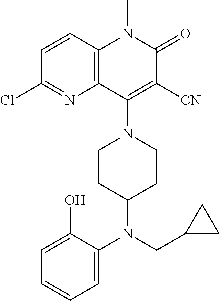 [SMILES: Cn1c(=O)c(C#N)c(N2CCC(N(CC3CC3)c3ccccc3O)CC2)c2nc(Cl)ccc21] | 6-chloro-4-(4-((cyclopropylmethyl)(2-hydroxyphenyl) amino)piperidin-1-yl)-1-methyl-2-oxo-1,2-dihydro-1,5- naphthyridine-3-carbonitrile |
| 1F | 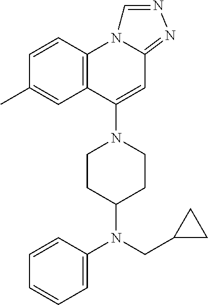 [SMILES: Cc1ccc2c(c1)c(N1CCC(N(CC3CC3)c3ccccc3)CC1)cc1nncn12] | N-(cyclopropylmethyl)-1-(7-methyl-[1,2,4] triazolo[4,3-a]quinolin-5-yl)-N-phenylpiperidin-4- amine |
| 2A | 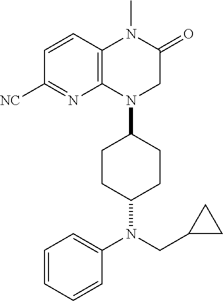 | trans-4-[4-[N-(cyclopropylmethyl)anilino]cyclohexyl]- 1-methyl-2-oxo-3H-pyrido[2,3-b]pyrazine-6- carbonitrile |
| 2B | 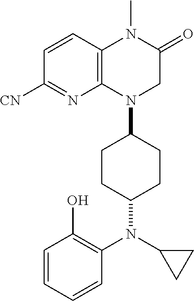 | 4-(trans-4-(cyclopropyl(2-hydroxyphenyl)amino) cyclohexyl)-1-methyl-2-oxo-1,2,3,4- tetrahydropyrido[3,2-b]pyrazine-6-carbonitrile |
| 2C | 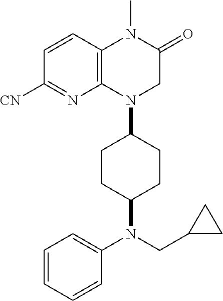 | cis-4-[4-[N-(cyclopropylmethyl)anilino]cyclohexyl]-1- methyl-2-oxo-3H-pyrido[2,3-b]pyrazine-6-carbonitrile |
| 3A | 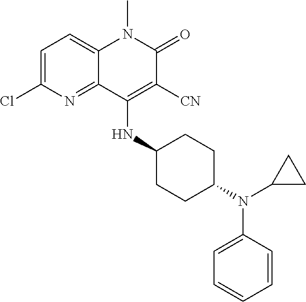 [SMILES: Cn1c(=O)c(C#N)c(N[C@H]2CC[C@H](N(c3ccccc3)C3CC3)CC2)c2nc(Cl)ccc21] | trans-6-chloro-4-((4-(cyclopropyl(phenyl)amino) cyclohexyl)amino)-1-methyl-2-oxo-1,2-dihydro-1,5- naphthyridine-3-carbonitrile |
| 3B | 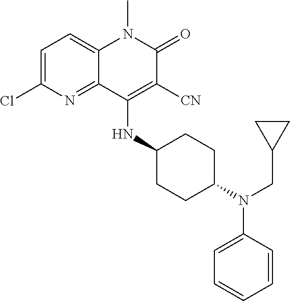 [SMILES: Cn1c(=O)c(C#N)c(N[C@H]2CC[C@H](N(CC3CC3)c3ccccc3)CC2)c2nc(Cl)ccc21] | trans-6-chloro-4-((4-((cyclopropylmethyl)(phenyl) amino)cyclohexyl)amino)-1-methyl-2-oxo-1,2- dihydro-1,5-naphthyridine-3-carbonitrile |
| 3C | 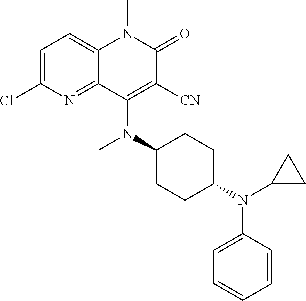 [SMILES: Cn1c(=O)c(C#N)c(N(C)[C@H]2CC[C@H](N(c3ccccc3)C3CC3)CC2)c2nc(Cl)ccc21] | trans-6-chloro-4-((4-(cyclopropyl(phenyl)amino) cyclohexyl)(methyl)amino)-1-methyl-2-oxo-1,2- dihydro-1,5-naphthyridine-3-carbonitrile |
| 3D | 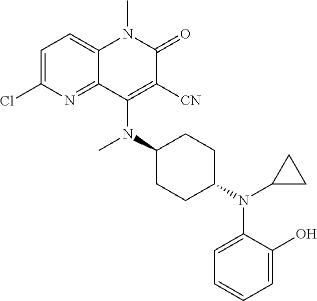 [SMILES: Cn1c(=O)c(C#N)c(N(C)[C@H]2CC[C@H](N(c3ccccc3O)C3CC3)CC2)c2nc(Cl)ccc21] | trans-6-chloro-4-((4-(cyclopropyl(2-hydroxyphenyl) amino)cyclohexyl)(methyl)amino)-1-methyl-2-oxo- 1,2-dihydro-1,5-naphthyridine-3-carbonitrile |
| 3E | 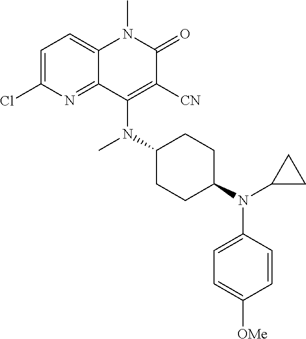 [SMILES: COc1ccc(N(C2CC2)[C@H]2CC[C@H](N(C)c3c(C#N)c(=O)n(C)c4ccc(Cl)nc34)CC2)cc1] | trans-6-chloro-4-((4-(cyclopropyl(4-methoxyphenyl) amino)cyclohexyl)(methyl)amino)-1-methyl-2-oxo- 1,2-dihydro-1,5-naphthyridine-3-carbonitrile |
| 3F | 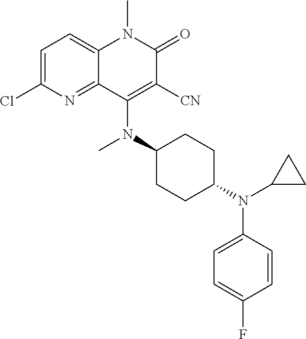 [SMILES: Cn1c(=O)c(C#N)c(N(C)[C@H]2CC[C@H](N(c3ccc(F)cc3)C3CC3)CC2)c2nc(Cl)ccc21] | trans-6-chloro-4-((4-(cyclopropyl(4-fluorophenyl) amino)cyclohexyl)(methyl)amino)-1-methyl-2-oxo- 1,2-dihydro-1,5-naphthyridine-3-carbonitrile |
| 3G | 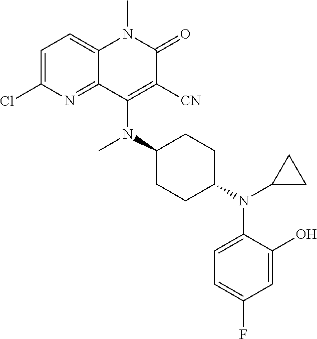 [SMILES: Cn1c(=O)c(C#N)c(N(C)[C@H]2CC[C@H](N(c3ccc(F)cc3O)C3CC3)CC2)c2nc(Cl)ccc21] | trans-6-chloro-4-((4-(cyclopropyl(4-fluoro-2- hydroxyphenyl)amino)cyclohexyl)(methyl)amino)-1- methyl-2-oxo-1,2-dihydro-1,5-naphthyridine-3- carbonitrile |
| 3H | 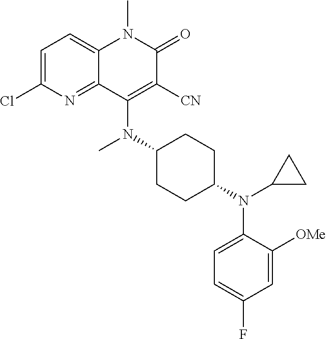 [SMILES: COc1cc(F)ccc1N(C1CC1)[C@H]1CC[C@@H](N(C)c2c(C#N)c(=O)n(C)c3ccc(Cl)nc23)CC1] | cis-6-chloro-4-((4-(cyclopropyl(4-fluoro-2- methoxyphenyl)amino)cyclohexyl)(methyl)amino)-1- methyl-2-oxo-1,2-dihydro-1,5-naphthyridine-3- carbonitrile |
| 3I | 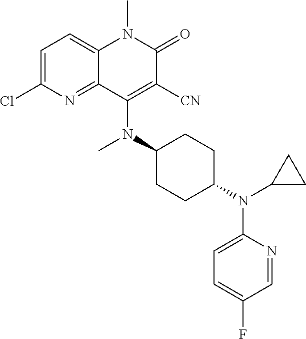 [SMILES: Cn1c(=O)c(C#N)c(N(C)[C@H]2CC[C@H](N(c3ccc(F)cn3)C3CC3)CC2)c2nc(Cl)ccc21] | trans-6-chloro-4-[[4-[cyclopropyl-(5-fluoro-2-pyridyl) amino]cyclohexyl]-methyl-amino]-1-methyl-2-oxo- 1,5-naphthyridine-3-carbonitrile |
| 3J | 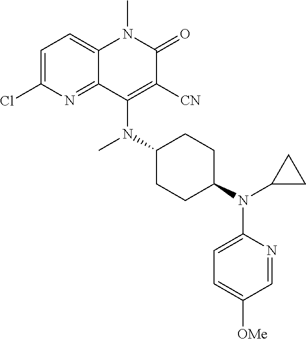 [SMILES: COc1ccc(N(C2CC2)[C@H]2CC[C@H](N(C)c3c(C#N)c(=O)n(C)c4ccc(Cl)nc34)CC2)nc1] | trans-6-chloro-4-((4-(cyclopropyl(5-methoxypyridin-2- yl)amino)cyclohexyl)(methyl)amino)-1-methyl-2-oxo- 1,2-dihydro-1,5-naphthyridine-3-carbonitrile |
| 3K | 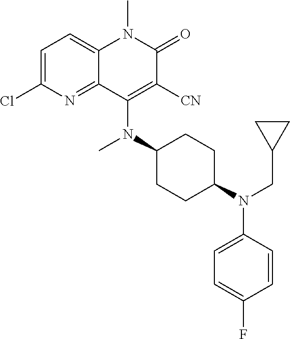 [SMILES: Cn1c(=O)c(C#N)c(N(C)[C@H]2CC[C@@H](N(CC3CC3)c3ccc(F)cc3)CC2)c2nc(Cl)ccc21] | cis-6-Chloro-4-((4-((cyclopropylmethyl)(4- fluorophenyl)amino)cyclohexyl)amino)-1-methyl-2- oxo-1,2-dihydro-1,5-naphthyridine-3-carbonitrile |
| 3L | 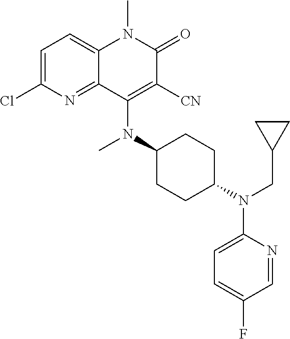 [SMILES: Cn1c(=O)c(C#N)c(N(C)[C@H]2CC[C@H](N(CC3CC3)c3ccc(F)cn3)CC2)c2nc(Cl)ccc21] | trans-6-chloro-4-((4-((cyclopropylmethyl)(5- fluoropyridin-2-yl)amino)cyclohexyl)(methyl)amino)- 1-methyl-2-oxo-1,2-dihydro-1,5-naphthyridine-3- carbonitrile |
| 3M | 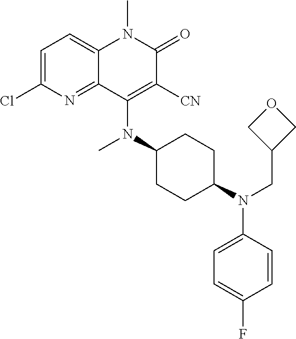 [SMILES: Cn1c(=O)c(C#N)c(N(C)[C@H]2CC[C@@H](N(CC3COC3)c3ccc(F)cc3)CC2)c2nc(Cl)ccc21] | cis-6-Chloro-4-[[4-(N-4-fluorophenyl-N-oxetan-3- ylmethylanilino)cyclohexyl]-methylamino]-1-methyl- 2-oxo-1,2-dihydro-1,5-naphthyridine-3-carbonitrile |
| 3N | 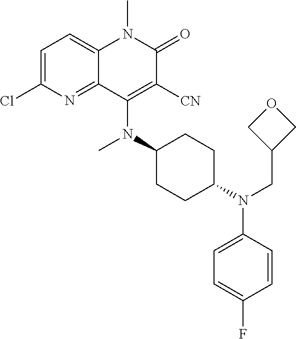 [SMILES: Cn1c(=O)c(C#N)c(N(C)[C@H]2CC[C@H](N(CC3COC3)c3ccc(F)cc3)CC2)c2nc(Cl)ccc21] | trans-6-Chloro-4-[[4-(N-4-fluorophenyl-N-oxetan-3- ylmethylanilino)cyclohexyl]-methylamino]-1-methyl- 2-oxo-1,2-dihydro-1,5-naphthyridine-3-carbonitrile |
| 3O | 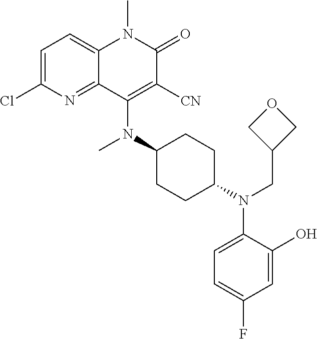 [SMILES: Cn1c(=O)c(C#N)c(N(C)[C@H]2CC[C@H](N(CC3COC3)c3ccc(F)cc3O)CC2)c2nc(Cl)ccc21] | trans-6-chloro-4-((-4-((4-fluoro-2-hydroxyphenyl) (oxetan-3-ylmethyl)amino)cyclohexyl)(methyl)amino)- 1-methyl-2-oxo-1,2-dihydro-1,5-naphthyridine-3- carbonitrile |
| 3Q | 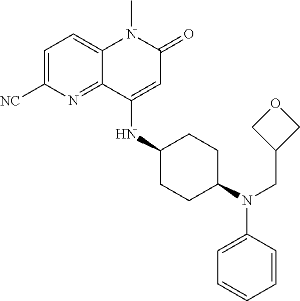 [SMILES: Cn1c(=O)cc(N[C@H]2CC[C@@H](N(CC3COC3)c3ccccc3)CC2)c2nc(C#N)ccc21] | cis-5-methyl-8-((4-((oxetan-3-ylmethyl)(phenyl)amino) cyclohexyl)amino)-6-oxo-5,6-dihydro-1,5- naphthyridine-2-carbonitrile |
| 3R | 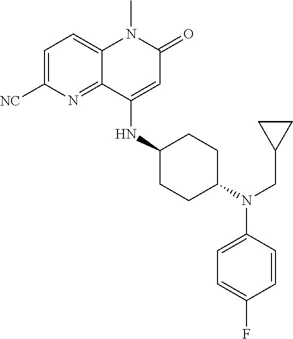 [SMILES: Cn1c(=O)cc(N[C@H]2CC[C@H](N(CC3CC3)c3ccc(F)cc3)CC2)c2nc(C#N)ccc21] | trans-8-((4-((cyclopropylmethyl)(4-fluorophenyl) amino)cyclohexyl)amino)-5-methyl-6-oxo-5,6- dihydro-1,5-naphthyridine-2-carbonitrile |
| 3S | 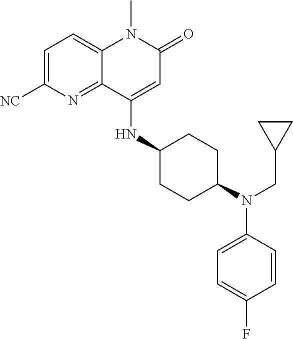 [SMILES: Cn1c(=O)cc(N[C@H]2CC[C@@H](N(CC3CC3)c3ccc(F)cc3)CC2)c2nc(C#N)ccc21] | cis-8-((4-((cyclopropylmethyl)(4-fluorophenyl)amino) cyclohexyl)amino)-5-methyl-6-oxo-5,6-dihydro-1,5- naphthyridine-2-carbonitrile |
| 3T | 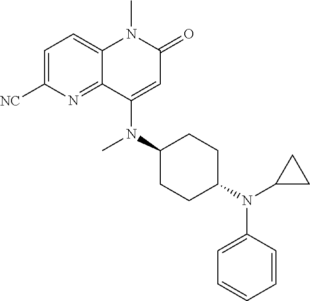 [SMILES: Cn1c(=O)cc(N(C)[C@H]2CC[C@H](N(c3ccccc3)C3CC3)CC2)c2nc(C#N)ccc21] | trans-8-((4-(cyclopropyl(phenyl)amino)cyclohexyl) (methyl)amino)-5-methyl-6-oxo-5,6-dihydro-1,5- naphthyridine-2-carbonitrile |
| 3U | 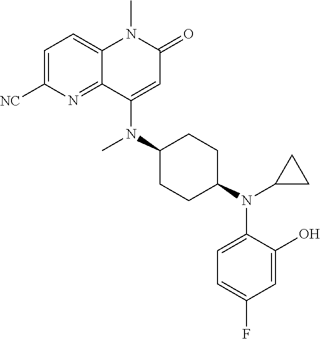 [SMILES: Cn1c(=O)cc(N(C)[C@H]2CC[C@@H](N(c3ccc(F)cc3O)C3CC3)CC2)c2nc(C#N)ccc21] | cis-8-[[4-(N-cyclopropyl-4-fluoro-2-methoxy-anilino) cyclohexyl]-methyl-amino]-5-methyl-6-oxo-1,5- naphthyridine-2-carbonitrile |
| 3V | 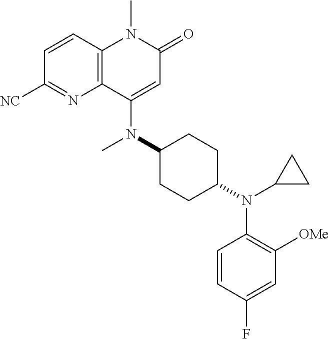 | trans-8-[[4-(N-cyclopropyl-4-fluoro-2-methoxy- anilino)cyclohexyl]-methyl-amino]-5-methyl-6-oxo- 1,5-naphthyridine-2-carbonitrile |
| 3W | 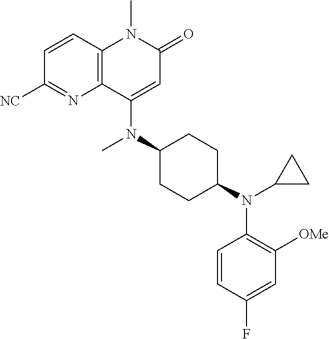 [SMILES: COc1cc(F)ccc1N(C1CC1)[C@H]1CC[C@@H](N(C)c2cc(=O)n(C)c3ccc(C#N)nc23)CC1] | cis-8-[[4-(N-cyclopropyl-4-fluoro-2-methoxy-anilino) cyclohexyl]-methyl-amino]-5-methyl-6-oxo-1,5- naphthyridine-2-carbonitrile |
| 3X | 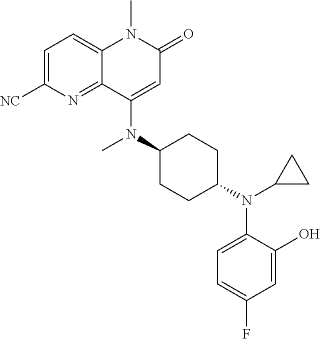 [SMILES: Cn1c(=O)cc(N(C)[C@H]2CC[C@H](N(c3ccc(F)cc3O)C3CC3)CC2)c2nc(C#N)ccc21] | trans-8-((4-(cyclopropyl(4-fluoro-2-hydroxyphenyl) amino)cyclohexyl)(methyl)amino)-5-methyl-6-oxo- 5,6-dihydro-1,5-naphthyridine-2-carbonitrile |
| 3Y | 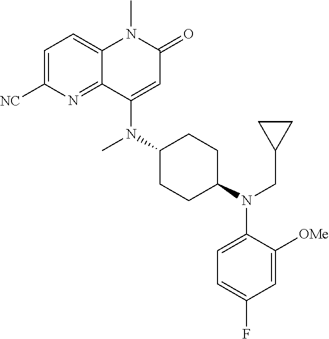 [SMILES: COc1cc(F)ccc1N(CC1CC1)[C@H]1CC[C@H](N(C)c2cc(=O)n(C)c3ccc(C#N)nc23)CC1] | trans-8-[[4-(N-cyclopropylmethyl-N-4-fluoro-2- methoxyphenyl-anilino)cyclohexyl]-methyl-amino]-5- methyl-6-oxo-5,6-dihydro-1,5-naphthyridine-2- carbonitrile |
| 3Z | 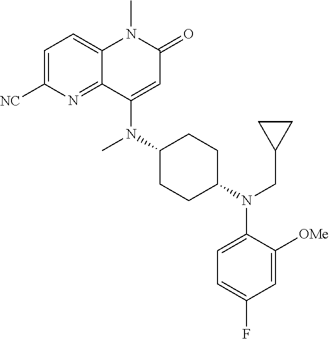 [SMILES: COc1cc(F)ccc1N(CC1CC1)[C@H]1CC[C@@H](N(C)c2cc(=O)n(C)c3ccc(C#N)nc23)CC1] | cis-8-[[4-(N-cyclopropylmethyl-N-4-fluoro-2- methoxyphenyl-anilino)cyclohexyl]-methyl-amino]-5- methyl-6-oxo-5,6-dihydro-1,5-naphthyridine-2- carbonitrile |
| 3AA | 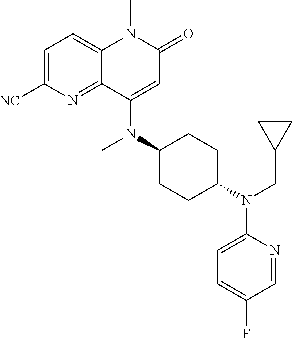 [SMILES: Cn1c(=O)cc(N(C)[C@H]2CC[C@H](N(CC3CC3)c3ccc(F)cn3)CC2)c2nc(C#N)ccc21] | trans-8-[[4-[cyclopropylmethyl-(5-fluoro-2-pyridyl) amino]cyclohexyl]-methyl-amino]-5-methyl-6-oxo- 1,5-naphthyridine-2-carbonitrile |
| 3BB | 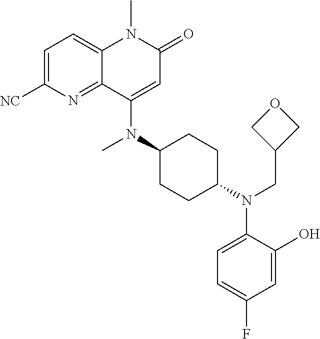 [SMILES: Cn1c(=O)cc(N(C)[C@H]2CC[C@H](N(CC3COC3)c3ccc(F)cc3O)CC2)c2nc(C#N)ccc21] | trans-8-((4-((4-fluoro-2-hydroxyphenyl)(oxetan-3- ylmethyl)amino)cyclohexyl)(methyl)amino)-5-methyl- 6-oxo-5,6-dihydro-1,5-naphthyridine-2-carbonitrile |
| 3CC | 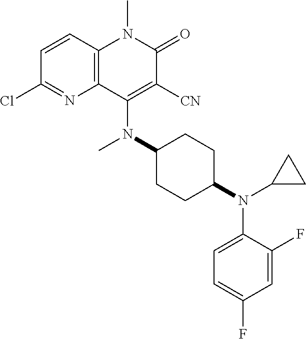 | cis-6-Chloro-4-[[4-(N-cyclopropyl-2,4-difluoro- anilino)cyclohexyl]-methyl-amino]-1-methyl-2-oxo- 1,5-naphthyridine-3-carbonitrile |
| 4A | 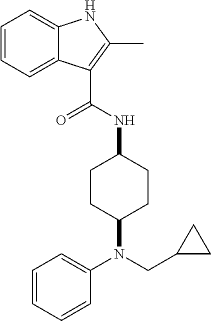 | cis-N-4-((cyclopropylmethyl)(phenyl)amino) cyclohexyl)-2-methyl-1H-indole-3-carboxamide |
| 4B | 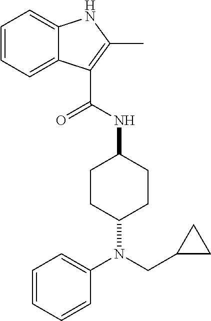 | trans-N-4-((cyclopropylmethyl)(phenyl)amino) cyclohexyl)-2-methyl-1H-indole-3-carboxamide |
| 4C | 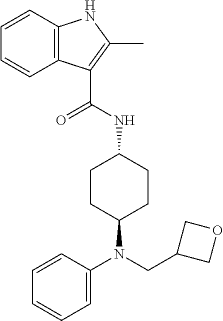 [SMILES: Cc1[nH]c2ccccc2c1C(=O)N[C@H]1CC[C@H](N(CC2COC2)c2ccccc2)CC1] | trans-2-methyl-N-(4-((oxetan-3-ylmethyl)(phenyl) amino)cyclohexyl)-1H-indole-3-carboxamide |
| 4D | 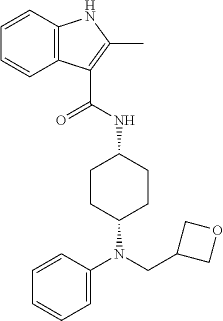 [SMILES: Cc1[nH]c2ccccc2c1C(=O)N[C@H]1CC[C@@H](N(CC2COC2)c2ccccc2)CC1] | cis-2-methyl-N-(4-((oxetan-3-ylmethyl)(phenyl)amino) cyclohexyl)-1H-indole-3-carboxamide |
| 4E | 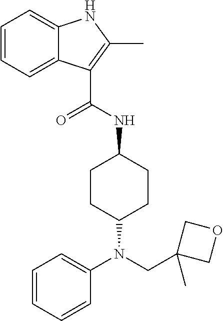 | trans-2-methyl-N-(4-(((3-methyloxetan-3-yl)methyl) (phenyl)amino)cyclohexyl)-1H-indole-3-carboxamide |
| 4F | 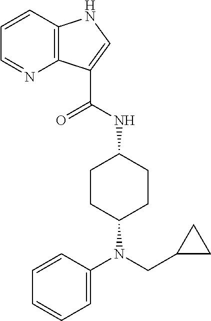 [SMILES: O=C(N[C@H]1CC[C@@H](N(CC2CC2)c2ccccc2)CC1)c1c[nH]c2cccnc12] | cis-N-(4-((cyclopropylmethyl)(phenyl)amino) cyclohexyl)-1H-pyrrolo[3,2-b]pyridine-3-carboxamide |
| 4G | 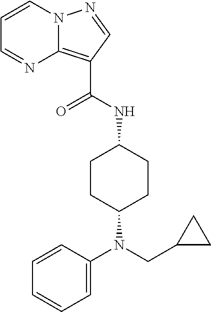 [SMILES: O=C(N[C@H]1CC[C@@H](N(CC2CC2)c2ccccc2)CC1)c1cnn2cccnc12] | cis-N-(4-((cyclopropylmethyl)(phenyl)amino) cyclohexyl)pyrazolo[1,5-a]pyrimidine-3-carboxamide |
| 5 | 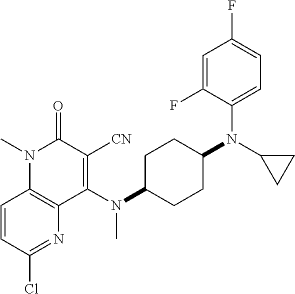 | cis-6-chloro-4-((-4-(cyclopropyl(2,4-difluorophenyl) amino)cyclohexyl)(methyl)amino)-1-methyl-2-oxo- 1,2-dihydro-1,5-naphthyridine-3-carbonitrile |
| 6 | 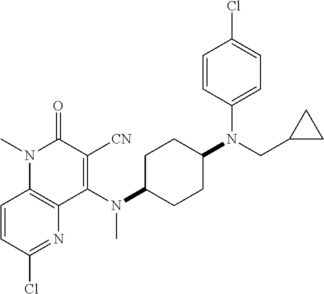 | cis-6-chloro-4-((4-((4-chlorophenyl) (cyclopropylmethyl)amino)cyclohexyl)(methyl) amino)-1-methyl-2-oxo-1,2-dihydro-1,5- naphthyridine-3-carbonitrile |
| 7 | 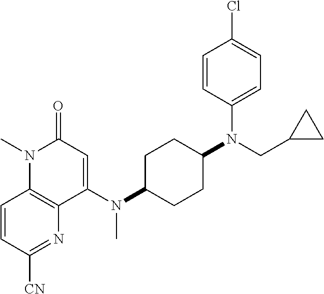 | cis-8-((4-((4-chlorophenyl)(cyclopropylmethyl) amino)cyclohexyl)(methyl)amino)-5-methyl-6-oxo- 5,6-dihydro-1,5-naphthyridine-2-carbonitrile |
| 8 | 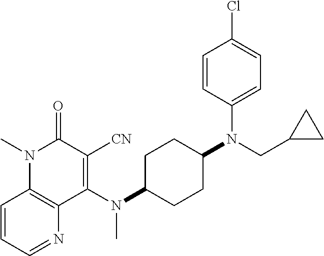 | cis-4-((4-((4-chlorophenyl)(cyclopropylmethyl) amino)cyclohexyl)(methyl)amino)-1-methyl-2-oxo- 1,2-dihydro-1,5-naphthyridine-3-carbonitrile |
| 9 | 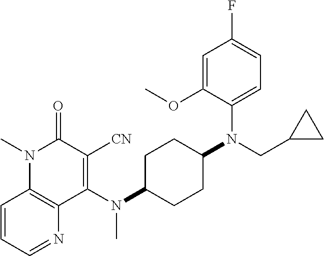 | cis-4-((4-((cyclopropylmethyl)(4-fluoro-2- methoxyphenyl)amino)cyclohexyl)(methyl)amino)-1- methyl-2-oxo-1,2-dihydro-1,5-naphthyridine-3- carbonitrile |
| 10 | 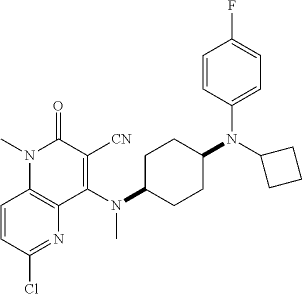 | cis-6-chloro-4-((4-(cyclobutyl(4-fluorophenyl)amino) cyclohexyl)(methyl)amino)-1-methyl-2-oxo-1,2- dihydro-1,5-naphthyridine-3-carbonitrile |
| 11 | 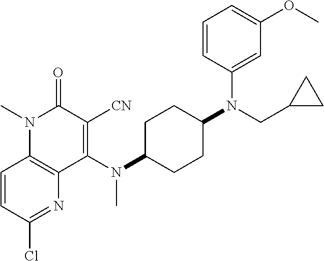 | cis-6-chloro-4-((4-((cyclopropylmethyl)(3- methoxyphenyl)amino)cyclohexyl)(methyl)amino)-1- methyl-2-oxo-1,2-dihydro-1,5-naphthyridine-3- carbonitrile |
| 12 | 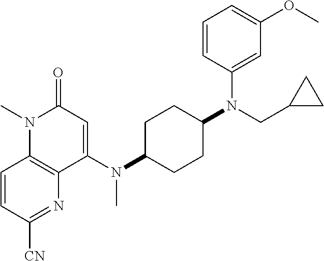 | cis-8-((4-((cyclopropylmethyl)(3-methoxyphenyl) amino)cyclohexyl)(methyl)amino)-5-methyl-6-oxo- 5,6-dihydro-1,5-naphthyridine-2-carbonitrile |
| 13 | 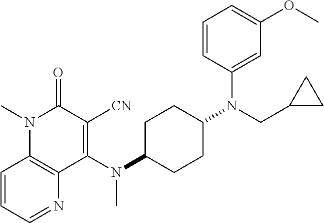 | trans-4-((4-((cyclopropylmethyl)(3-methoxyphenyl) amino)cyclohexyl)(methyl)amino)-1-methyl-2-oxo- 1,2-dihydro-1,5-naphthyridine-3-carbonitrile |
| 14 | 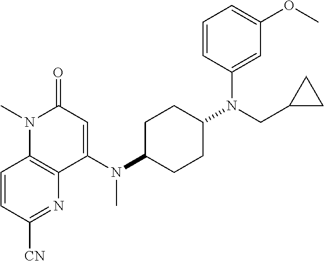 | trans-8-((4-((cyclopropylmethyl)(3-methoxyphenyl) amino)cyclohexyl)(methyl)amino)-5-methyl-6-oxo- 5,6-dihydro-1,5-naphthyridine-2-carbonitrile |
| 15 | 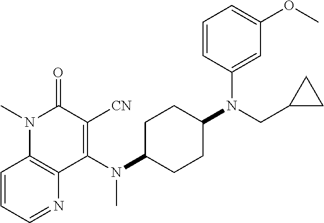 | cis-4-((4-((cyclopropylmethyl)(3-methoxyphenyl) amino)cyclohexyl)(methyl)amino)-1-methyl-2-oxo- 1,2-dihydro-1,5-naphthyridine-3-carbonitrile |
| 16 | 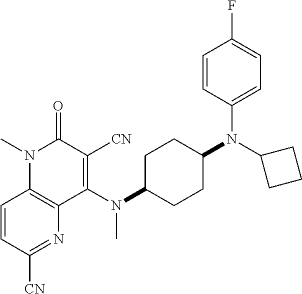 | cis-6-chloro-4-((4-(cyclobutyl(4-fluorophenyl)amino) cyclohexyl)(methyl)amino)-1-methyl-2-oxo-1,2- dihydro-1,5-naphthyridine-3-carbonitrile |
| 17 | 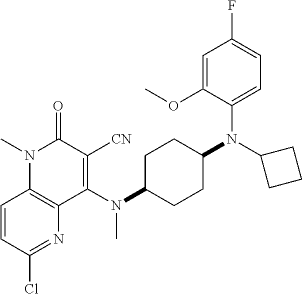 | cis-6-chloro-4-((4-(cyclobutyl(4-fluoro-2- methoxyphenyl)amino)cyclohexyl)(methyl)amino)-1- methyl-2-oxo-1,2-dihydro-1,5-naphthyridine-3- carbonitrile |
| 18 | 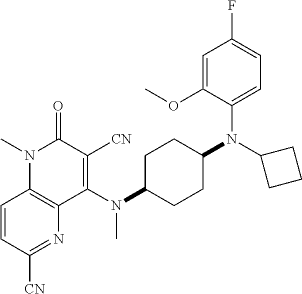 | cis-8-((4-(cyclobutyl(4-fluoro-2-methoxyphenyl) amino)cyclohexyl)(methyl)amino)-5-methyl-6-oxo- 5,6-dihydro-1,5-naphthyridine-2,7-dicarbonitrile |
| 19 | 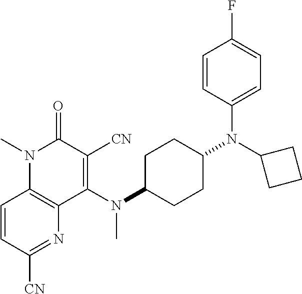 | trans-8-((4-(cyclobutyl(4-fluorophenyl)amino) cyclohexyl)(methyl)amino)-5-methyl-6-oxo-5,6- dihydro-1,5-naphthyridine-2,7-dicarbonitrile |
| 20 | 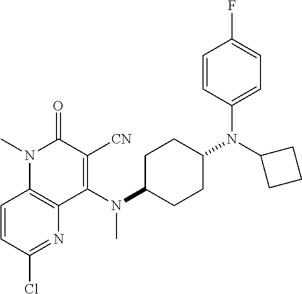 | trans-6-chloro-4-((4-(cyclobutyl(4-fluorophenyl) amino)cyclohexyl)(methyl)amino)-1-methyl-2-oxo- 1,2-dihydro-1,5-naphthyridine-3-carbonitrile |
| 21 | 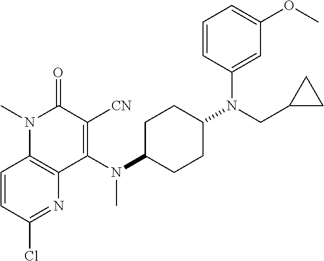 | trans-6-chloro-4-((4-((cyclopropylmethyl)(3- methoxyphenyl)amino)cyclohexyl)(methyl)amino)-1- methyl-2-oxo-1,2-dihydro-1,5-naphthyridine-3- carbonitrile |
| 22 | 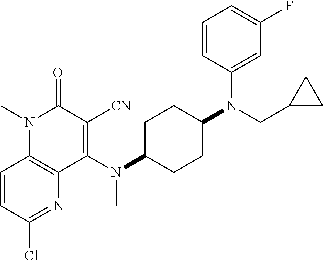 | cis-6-chloro-4-((4-((cyclopropylmethyl)(3- fluorophenyl)amino)cyclohexyl)(methyl)amino)-1- methyl-2-oxo-1,2-dihydro-1,5-naphthyridine-3- carbonitrile |
| 23 | 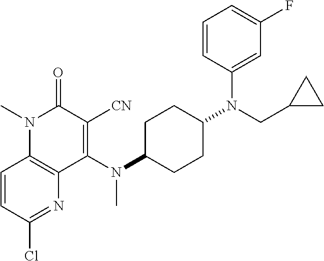 | trans-6-chloro-4-((4-((cyclopropylmethyl)(3- fluorophenyl)amino)cyclohexyl)(methyl)amino)-1- methyl-2-oxo-1,2-dihydro-1,5-naphthyridine-3- carbonitrile |
| 24 | 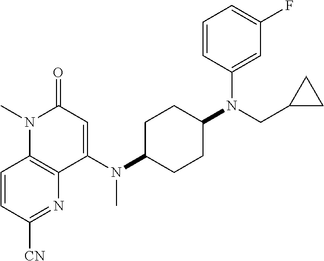 | cis-8-((4-((cyclopropylmethyl)(3-fluorophenyl) amino)cyclohexyl)(methyl)amino)-5-methyl-6-oxo- 5,6-dihydro-1,5-naphthyridine-2-carbonitrile |
| 25 | 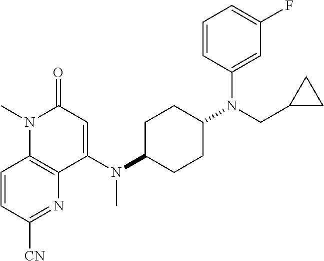 | trans-8-((4-((cyclopropylmethyl)(3-fluorophenyl) amino)cyclohexyl)(methyl)amino)-5-methyl-6-oxo- 5,6-dihydro-1,5-naphthyridine-2-carbonitrile |
| 26 | 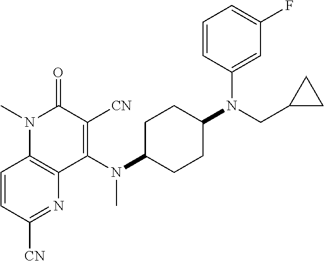 | cis-8-((4-((cyclopropylmethyl)(3-fluorophenyl) amino)cyclohexyl)(methyl)amino)-5-methyl-6-oxo- 5,6-dihydro-1,5-naphthyridine-2,7-dicarbonitrile |
| 27 | 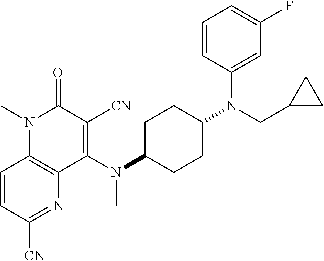 | trans-8-((4-((cyclopropylmethyl)(3-fluorophenyl) amino)cyclohexyl)(methyl)amino)-5-methyl-6-oxo- 5,6-dihydro-1,5-naphthyridine-2,7-dicarbonitrile |
| 28 | 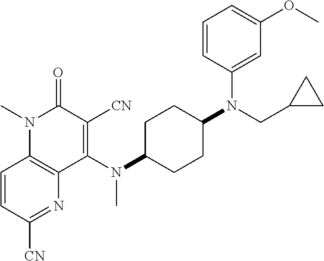 | cis-8-((4-((cyclopropylmethyl)(3-methoxyphenyl) amino)cyclohexyl)(methyl)amino)-5-methyl-6-oxo- 5,6-dihydro-1,5-naphthyridine-2,7-dicarbonitrile |
| 29 | 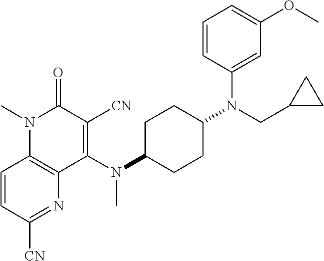 | trans-8-((4-((cyclopropylmethyl)(3-methoxyphenyl) amino)cyclohexyl)(methyl)amino)-5-methyl-6-oxo- 5,6-dihydro-1,5-naphthyridine-2,7-dicarbonitrile |
| 30 | 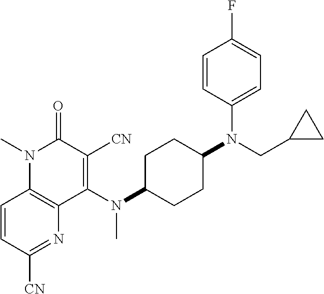 | cis-8-((4-((cyclopropylmethyl)(4-fluorophenyl) amino)cyclohexyl)(methyl)amino)-5-methyl-6-oxo- 5,6-dihydro-1,5-naphthyridine-2,7-dicarbonitrile |
| 31 | 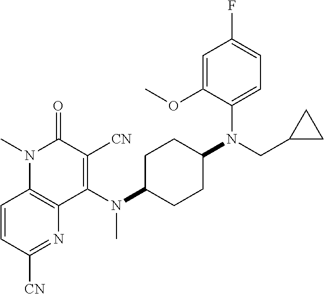 | cis-8-((4-((cyclopropylmethyl)(4-fluoro-2- methoxyphenyl)amino)cyclohexyl)(methyl)amino)-5- methyl-6-oxo-5,6-dihydro-1,5-naphthyridine-2,7- dicarbonitrile |
| 32 | 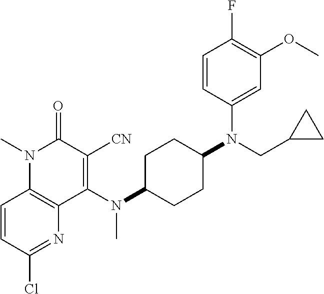 | cis-6-chloro-4-((4-((cyclopropylmethyl)(4-fluoro-3- methoxyphenyl)amino)cyclohexyl)(methyl)amino)-1- methyl-2-oxo-1,2-dihydro-1,5-naphthyridine-3- carbonitrile |
| 33 | 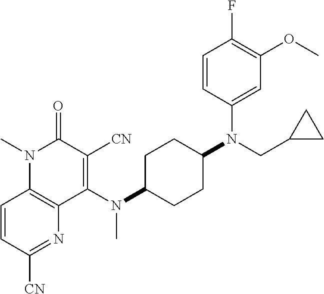 | cis-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2,7- dicarbonitrile |
| 34 | 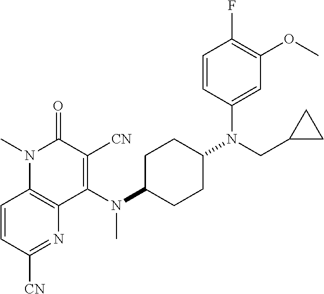 | trans-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2,7- dicarbonitrile |
| 35 | 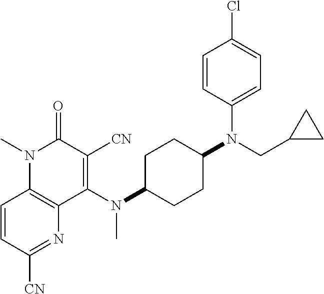 | cis-8-((4-((4-chlorophenyl)(cyclopropylmethyl) amino)cyclohexyl)(methyl)amino)-5-methyl-6-oxo- 5,6-dihydro-1,5-naphthyridine-2,7-dicarbonitrile |
| 36 | 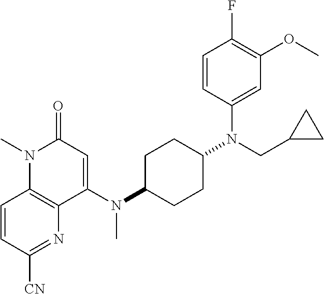 | trans-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2- carbonitrile |
| 37 | 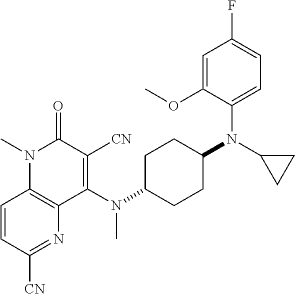 | trans-8-((4-(cyclopropyl(4-fluoro-2-methoxyphenyl) amino)cyclohexyl)(methyl)amino)-5-methyl-6-oxo- 5,6-dihydro-1,5-naphthyridine-2,7-dicarbonitrile |
| 38 | 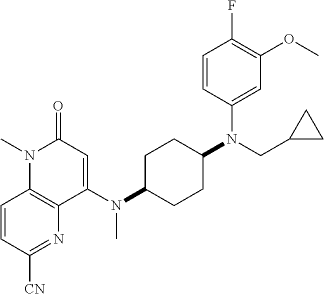 | cis-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2- carbonitrile |
| 39 | 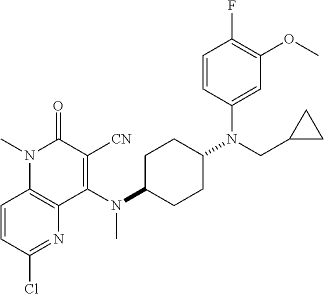 | trans-6-chloro-4-((4-((cyclopropylmethyl)(4-fluoro- 3-methoxyphenyl)amino)cyclohexyl)(methyl)amino)- 1-methyl-2-oxo-1,2-dihydro-1,5-naphthyridine-3- carbonitrile |
| 40 | 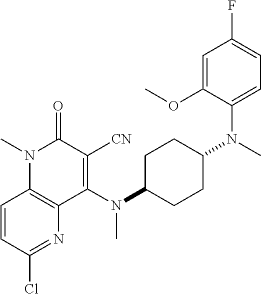 | trans-6-chloro-4-((4-((4-fluoro-2-methoxyphenyl) (methyl)amino)cyclohexyl)(methyl)amino)-1-methyl- 2-oxo-1,2-dihydro-1,5-naphthyridine-3-carbonitrile |
| 41 | 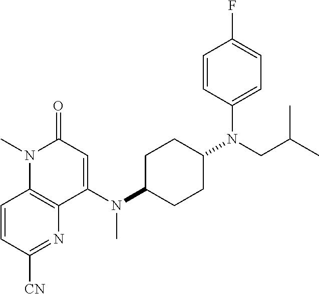 | trans-8-((-4-((4-fluorophenyl)(isobutyl)amino) cyclohexyl)(methyl)amino)-5-methyl-6-oxo-5,6- dihydro-1,5-naphthyridine-2-carbonitrile |
| 42 | 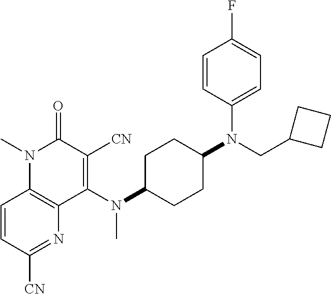 | cis-8-((-4-((cyclobutylmethyl)(4-fluorophenyl)amino) cyclohexyl)(methyl)amino)-5-methyl-6-oxo-5,6- dihydro-1,5-naphthyridine-2,7-dicarbonitrile |
| 43 | 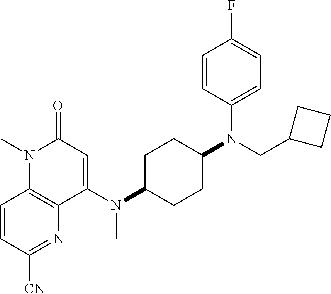 | cis-8-((-4-((cyclobutylmethyl)(4-fluorophenyl)amino) cyclohexyl)(methyl)amino)-5-methyl-6-oxo-5,6- dihydro-1,5-naphthyridine-2-carbonitrile |
| 44 | 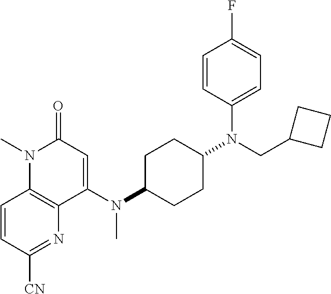 | trans-8-((-4-((cyclobutylmethyl)(4-fluorophenyl) amino)cyclohexyl)(methyl)amino)-5-methyl-6-oxo- 5,6-dihydro-1,5-naphthyridine-2-carbonitrile |
| 45 | 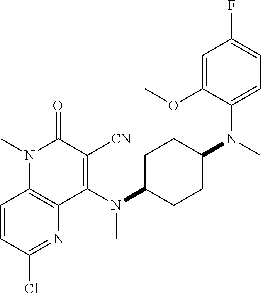 | cis-6-chloro-4-((4-((4-fluoro-2-methoxyphenyl) (methyl)amino)cyclohexyl)(methyl)amino)-1-methyl- 2-oxo-1,2-dihydro-1,5-naphthyridine-3-carbonitrile |
| 46 | 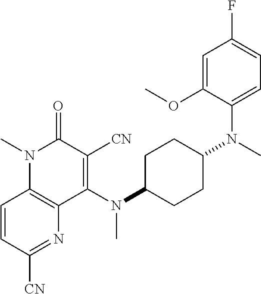 | trans-8-((4-((4-fluoro-2-methoxyphenyl)(methyl) amino)cyclohexyl)(methyl)amino)-5-methyl-6-oxo- 5,6-dihydro-1,5-naphthyridine-2,7-dicarbonitrile |
| 47 | 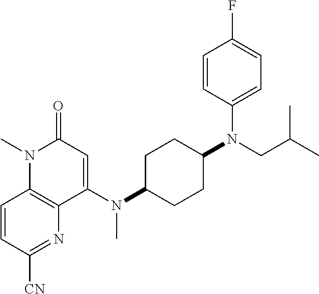 | cis-8-((-4-((4-fluorophenyl)(isobutyl)amino) cyclohexyl)(methyl)amino)-5-methyl-6-oxo-5,6- dihydro-1,5-naphthyridine-2-carbonitrile |
| 48 | 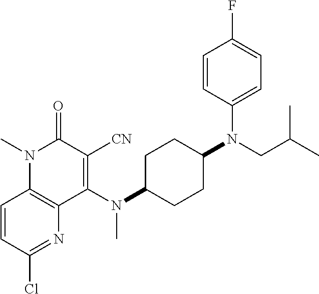 | cis-6-chloro-4-((-4-((4-fluorophenyl)(isobutyl)amino) cyclohexyl)(methyl)amino)-1-methyl-2-oxo-1,2- dihydro-1,5-naphthyridine-3-carbonitrile |
| 49 | 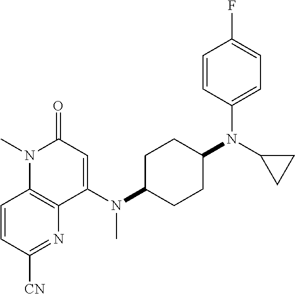 | cis-8-((4-(cyclopropyl(4-fluorophenyl)amino) cyclohexyl)(methyl)amino)-5-methyl-6-oxo-5,6- dihydro-1,5-naphthyridine-2-carbonitrile |
| 50 | 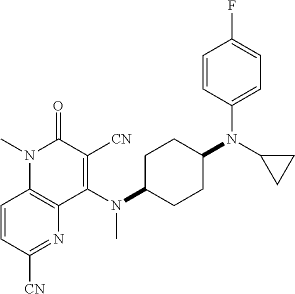 | cis-8-((4-(cyclopropyl(4-fluorophenyl)amino) cyclohexyl)(methyl)amino)-5-methyl-6-oxo-5,6- dihydro-1,5-naphthyridine-2,7-dicarbonitrile |
| 51 | 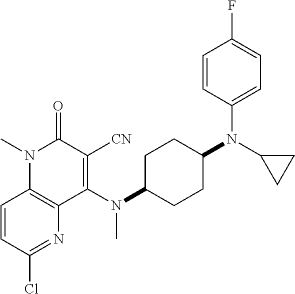 | cis-6-chloro-4-((4-(cyclopropyl(4-fluorophenyl) amino)cyclohexyl)(methyl)amino)-1-methyl-2-oxo- 1,2-dihydro-1,5-naphthyridine-3-carbonitrile |
| 52 | 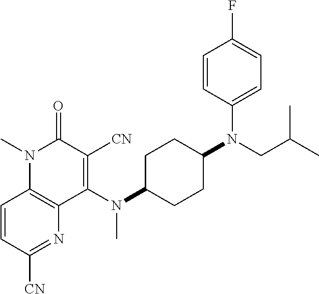 | cis-8-((-4-((4-fluorophenyl)(isobutyl)amino) cyclohexyl)(methyl)amino)-5-methyl-6-oxo-5,6- dihydro-1,5-naphthyridine-2,7-dicarbonitrile |
| 53 | 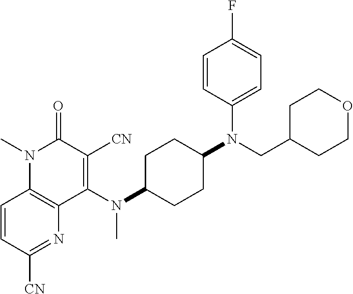 | cis-8-((-4-((4-fluorophenyl)((tetrahydro-2H-pyran-4- yl)methyl)amino)cyclohexyl)(methyl)amino)-5- methyl-6-oxo-5,6-dihydro-1,5-naphthyridine-2,7- dicarbonitrile |
| 54 | 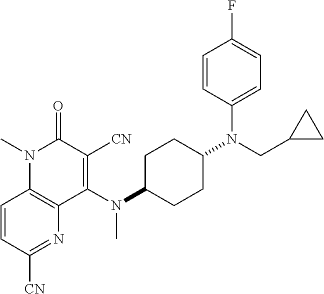 | trans-8-((4-((cyclopropylmethyl)(4-fluorophenyl) amino)cyclohexyl)(methyl)amino)-5-methyl-6-oxo- 5,6-dihydro-1,5-naphthyridine-2,7-dicarbonitrile |
| 55 | 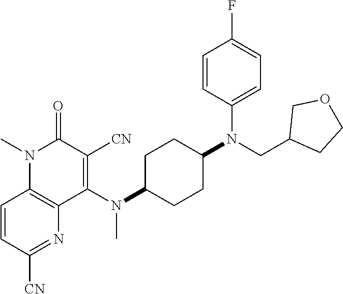 | cis-8-((4-((4-fluorophenyl)((tetrahydrofuran-3-yl) methyl)amino)cyclohexyl)(methyl)amino)-5-methyl- 6-oxo-5,6-dihydro-1,5-naphthyridine-2,7- dicarbonitrile |
| 56 | 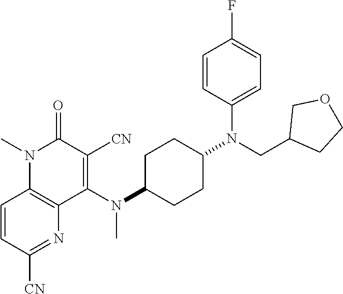 | trans-8-((4-((4-fluorophenyl)((tetrahydrofuran-3-yl) methyl)amino)cyclohexyl)(methyl)amino)-5-methyl- 6-oxo-5,6-dihydro-1,5-naphthyridine-2,7- dicarbonitrile |
| 57 | 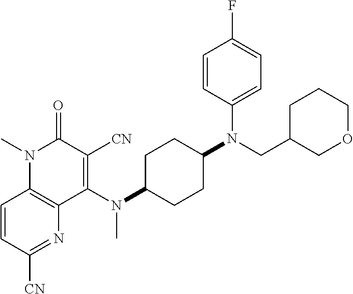 | cis-8-((4-((4-fluorophenyl)((tetrahydro-2H-pyran-3- yl)methyl)amino)cyclohexyl)(methyl)amino)-5- methyl-6-oxo-5,6-dihydro-1,5-naphthyridine-2,7- dicarbonitrile |
| 58 | 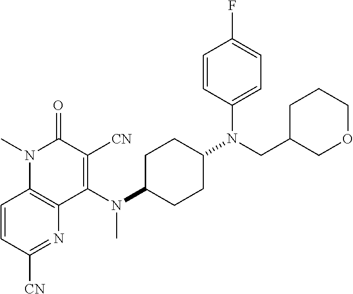 | trans-8-((4-((4-fluorophenyl)((tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 59 | 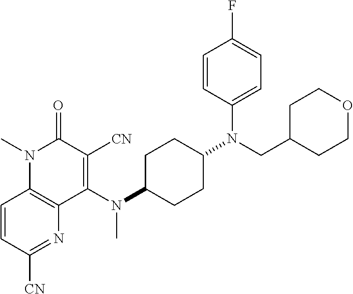 | trans-8-((-4-((4-fluorophenyl)((tetrahydro-2H-pyran- 4-yl)methyl)amino)cyclohexyl)(methyl)amino)-5- methyl-6-oxo-5,6-dihydro-1,5-naphthyridine-2,7- dicarbonitrile |
| 60 | 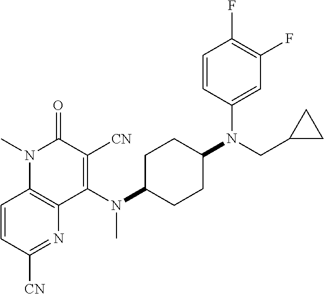 | cis-8-((4-((cyclopropylmethyl)(3,4-difluorophenyl) amino)cyclohexyl)(methyl)amino)-5-methyl-6-oxo- 5,6-dihydro-1,5-naphthyridine-2,7-dicarbonitrile |
| 61 | 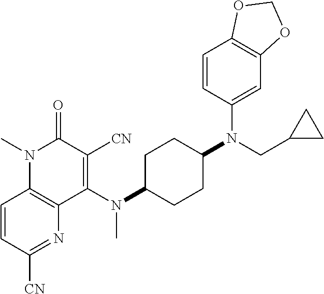 | cis-8-((4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 62 | 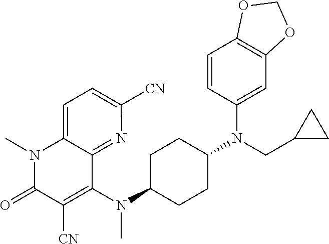 [SMILES: Cn1c(=O)c(C#N)c(N(C)[C@H]2CC[C@H](N(CC3CC3)c3ccc4c(c3)OCO4)CC2)c2nc(C#N)ccc21] | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 63 | 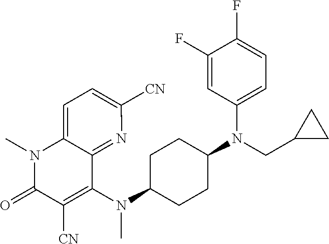 [SMILES: Cn1c(=O)c(C#N)c(N(C)[C@H]2CC[C@@H](N(CC3CC3)c3ccc(F)c(F)c3)CC2)c2nc(C#N)ccc21] | Cis-8-4-((cyclopropylmethyl)(3,4-difluorophenyl) amino)cyclohexyl)(methyl)amino)-5-methyl-6-oxo- 5,6-dihydro-1,5-naphthyridine-2,7-dicarbonitrile |
| 64 | 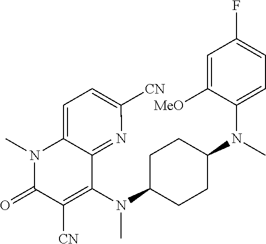 [SMILES: COc1cc(F)ccc1N(C)[C@H]1CC[C@@H](N(C)c2c(C#N)c(=O)n(C)c3ccc(C#N)nc23)CC1] | Cis-8-4-((4-fluoro-2-methoxyphenyl)(methyl)amino) cyclohexyl)(methyl)amino)-5-methyl-6-oxo-5,6- dihydro-1,5-naphthyridine-2,7-dicarbonitrile |
| 65 | 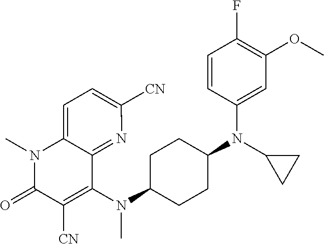 | cis-8-4-(cyclopropyl(4-fluoro-3-methoxyphenyl) amino)cyclohexyl)(methyl)amino)-5-methyl-6-oxo- 5,6-dihydro-1,5-naphthyridine-2,7-dicarbonitrile |
| 66 | 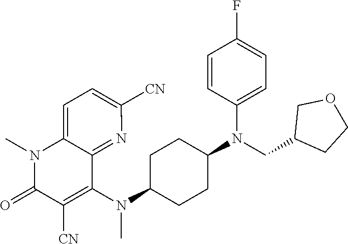 | cis-8-4-((4-fluorophenyl)(((R)-tetrahydrofuran-3-yl) methyl)amino)cyclohexyl)(methyl)amino)-5-methyl- 6-oxo-5,6-dihydro-1,5-naphthyridine-2,7- dicarbonitrile |
| 67 | 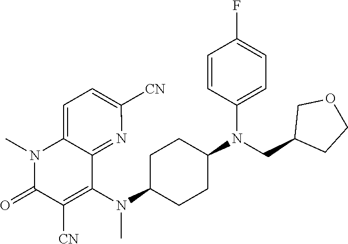 [SMILES: Cn1c(=O)c(C#N)c(N(C)[C@H]2CC[C@@H](N(C[C@@H]3CCOC3)c3ccc(F)cc3)CC2)c2nc(C#N)ccc21] | cis-8-4-((4-fluorophenyl)(((S)-tetrahydrofuran-3-yl) methyl)amino)cyclohexyl)(methyl)amino)-5-methyl- 6-oxo-5,6-dihydro-1,5-naphthyridine-2,7- dicarbonitrile |
| 68 | 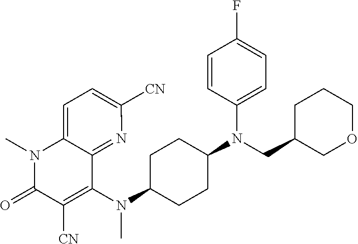 [SMILES: Cn1c(=O)c(C#N)c(N(C)[C@H]2CC[C@@H](N(C[C@H]3CCCOC3)c3ccc(F)cc3)CC2)c2nc(C#N)ccc21] | cis-8-4-((4-fluorophenyl)(((R)-tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 69 | 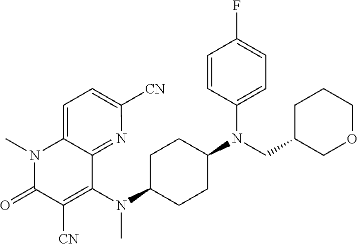 [SMILES: Cn1c(=O)c(C#N)c(N(C)[C@H]2CC[C@@H](N(C[C@@H]3CCCOC3)c3ccc(F)cc3)CC2)c2nc(C#N)ccc21] | cis-8-4-((4-fluorophenyl)(((R)-tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 70 | 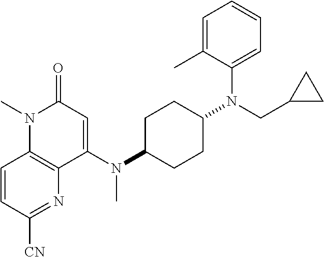 | trans-8-((4-((cyclopropylmethyl)(o-tolyl)amino) cyclohexyl)(methyl)amino)-5-methyl-6-oxo-5,6- dihydro-1,5-naphthyridine-2-carbonitrile |
| 71 | 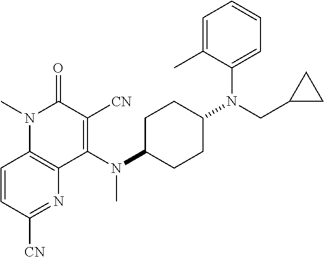 | trans-8-((4-((cyclopropylmethyl)(o-tolyl)amino) cyclohexyl)(methyl)amino)-5-methyl-6-oxo-5,6- dihydro-1,5-naphthyridine-2,7-dicarbonitrile |
| 72 | 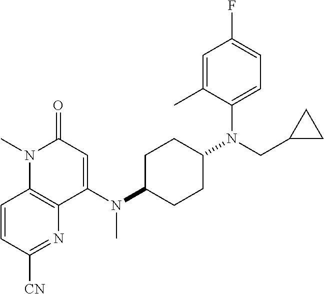 | trans-8-((4-((cyclopropylmethyl)(4-fluoro-2- methylphenyl)amino)cyclohexyl)(methyl)amino)-5- methyl-6-oxo-5,6-dihydro-1,5-naphthyridine-2- carbonitrile |
| 73 | 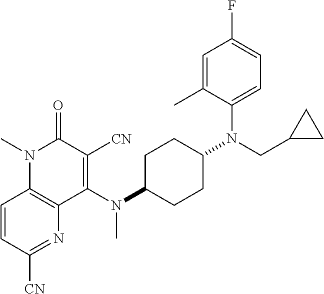 | trans-8-((4-((cyclopropylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 74 | 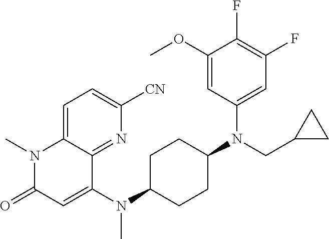 [SMILES: COc1cc(N(CC2CC2)[C@H]2CC[C@@H](N(C)c3cc(=O)n(C)c4ccc(C#N)nc34)CC2)cc(F)c1F] | cis-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2- carbonitrile |
| 75 | 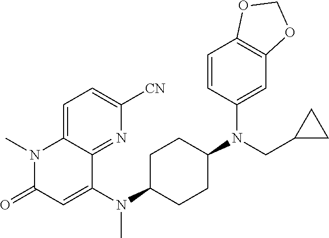 [SMILES: Cn1c(=O)cc(N(C)[C@H]2CC[C@@H](N(CC3CC3)c3ccc4c(c3)OCO4)CC2)c2nc(C#N)ccc21] | cis-8-4-(benzo[d][1,3]dioxol-5-yl(cyclopropylmethyl) amino)cyclohexyl)(methyl)amino)-5-methyl-6-oxo- 5,6-dihydro-1,5-naphthyridine-2-carbonitrile |
| 76 | 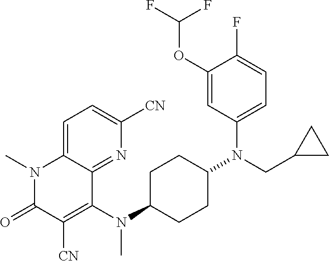 | trans-8-4-((cyclopropylmethyl)(3-(difluoromethoxy)- 4-fluorophenyl)amino)cyclohexyl)(methyl)amino)-5- methyl-6-oxo-5,6-dihydro-1,5-naphthyridine-2,7- dicarbonitrile |
| 77 | 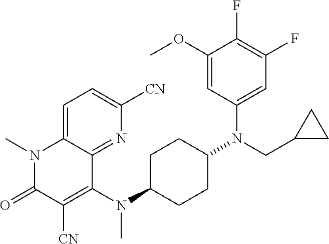 | trans-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2,7- dicarbonitrile |
| 78 | 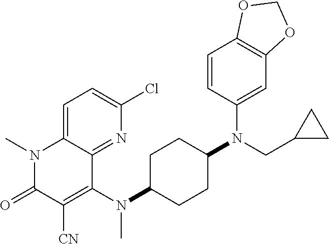 | cis-4-4-(benzo[d][1,3]dioxol-5-yl(cyclopropylmethyl) amino)cyclohexyl)(methyl)amino)-6-chloro-1- methyl-2-oxo-1,2-dihydro-1,5-naphthyridine-3- carbonitrile |
| 79 | 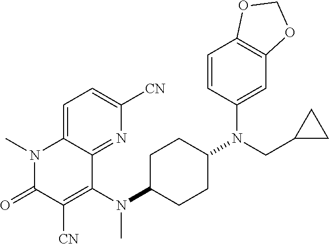 | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 80 | 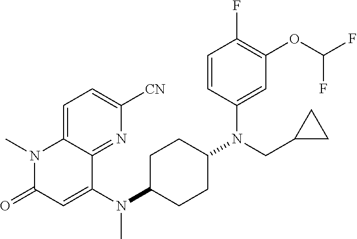 | trans-8-4-((cyclopropylmethyl)(3-(difluoromethoxy)- 4-fluorophenyl)amino)cyclohexyl)(methyl)amino)-5- methyl-6-oxo-5,6-dihydro-1,5-naphthyridine-2- carbonitrile |
| 81 | 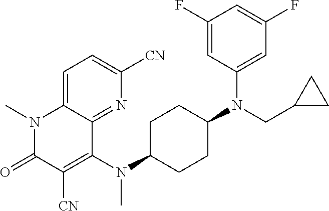 [SMILES: Cn1c(=O)c(C#N)c(N(C)[C@H]2CC[C@@H](N(CC3CC3)c3cc(F)cc(F)c3)CC2)c2nc(C#N)ccc21] | cis-8-4-((cyclopropylmethyl)(3,5-difluorophenyl) amino)cyclohexyl)(methyl)amino)-5-methyl-6-oxo- 5,6-dihydro-1,5-naphthyridine-2,7-dicarbonitrile |
| 82 | 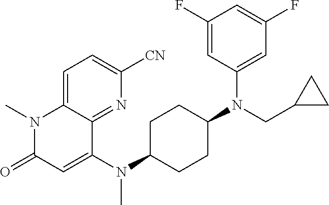 [SMILES: Cn1c(=O)cc(N(C)[C@H]2CC[C@@H](N(CC3CC3)c3cc(F)cc(F)c3)CC2)c2nc(C#N)ccc21] | cis-8-4-((cyclopropylmethyl)(3,5-difluorophenyl) amino)cyclohexyl)(methyl)amino)-5-methyl-6-oxo- 5,6-dihydro-1,5-naphthyridine-2-carbonitrile |
| 83 | 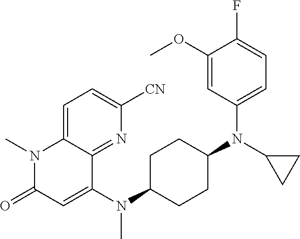 [SMILES: COc1cc(N(C2CC2)[C@H]2CC[C@@H](N(C)c3cc(=O)n(C)c4ccc(C#N)nc34)CC2)ccc1F] | cis-8-4-(cyclopropyl(4-fluoro-3-methoxyphenyl) amino)cyclohexyl)(methyl)amino)-5-methyl-6-oxo- 5,6-dihydro-1,5-naphthyridine-2-carbonitrile |
| 84 | 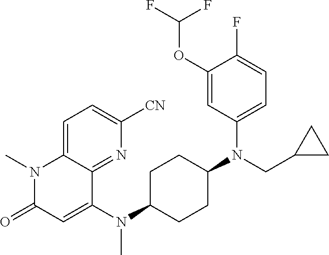 [SMILES: Cn1c(=O)cc(N(C)[C@H]2CC[C@@H](N(CC3CC3)c3ccc(F)c(OC(F)F)c3)CC2)c2nc(C#N)ccc21] | cis-8-4-((cyclopropylmethyl)(3-(difluoromethoxy)-4- fluorophenyl)amino)cyclohexyl)(methyl)amino)-5- methyl-6-oxo-5,6-dihydro-1,5-naphthyridine-2- carbonitrile |
| 85 | 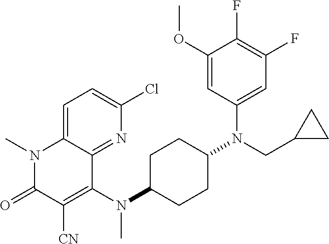 | trans-6-chloro-4-4-((cyclopropylmethyl)(3,4- difluoro-5-methoxyphenyl)amino)cyclohexyl) (methyl)amino)-1-methyl-2-oxo-1,2-dihydro-1,5- naphthyridine-3-carbonitrile |
| 86 | 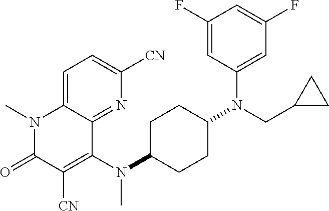 | trans-8-4-((cyclopropylmethyl)(3,5-difluorophenyl) amino)cyclohexyl)(methyl)amino)-5-methyl-6-oxo- 5,6-dihydro-1,5-naphthyridine-2,7-dicarbonitrile |
| 87 | 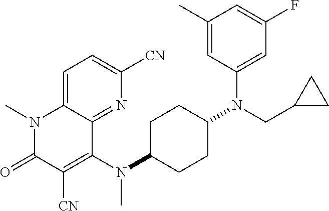 | trans-8-4-((cyclopropylmethyl)(3-fluoro-5- methylphenyl)amino)cyclohexyl)(methyl)amino)-5- methyl-6-oxo-5,6-dihydro-1,5-naphthyridine-2,7- dicarbonitrile |
| 88 | 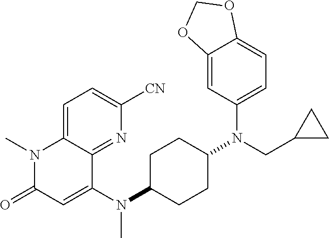 | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2-carbonitrile |
| 89 | 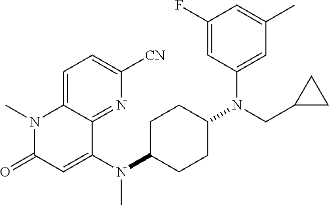 | trans-8-4-((cyclopropylmethyl)(3-fluoro-5- methylphenyl)amino)cyclohexyl)(methyl)amino)-5- methyl-6-oxo-5,6-dihydro-1,5-naphthyridine-2- carbonitrile |
| 90 | 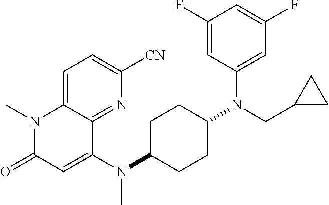 | trans-8-4-((cyclopropylmethyl)(3,5-difluorophenyl) amino)cyclohexyl)(methyl)amino)-5-methyl-6-oxo- 5,6-dihydro-1,5-naphthyridine-2-carbonitrile |
| 91 | 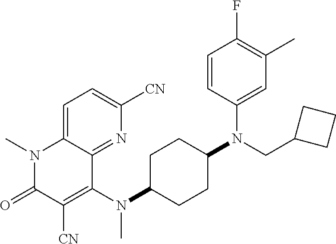 | cis-8-4-((cyclobutylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 92 | 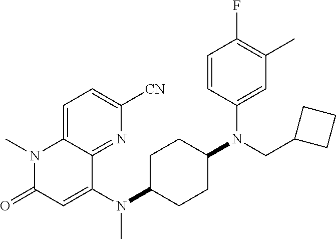 | cis-8-4-((cyclobutylmethyl)(4-fluoro-3- methylphenyl)amino)cyclohexyl)(methyl)amino)-5- methyl-6-oxo-5,6-dihydro-1,5-naphthyridine-2- carbonitrile |
| 93 | 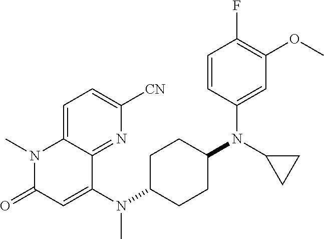 | trans-8-4-(cyclopropyl(4-fluoro-3-methoxyphenyl) amino)cyclohexyl)(methyl)amino)-5-methyl-6-oxo- 5,6-dihydro-1,5-naphthyridine-2-carbonitrile |
| 94 | 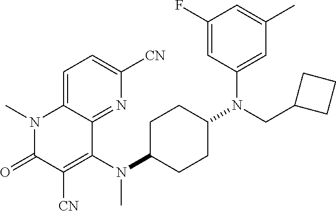 | trans-8-4-((cyclobutylmethyl)(3-fluoro-5- methylphenyl)amino)cyclohexyl)(methyl)amino)-5- methyl-6-oxo-5,6-dihydro-1,5-naphthyridine-2,7- dicarbonitrile |
| 95 | 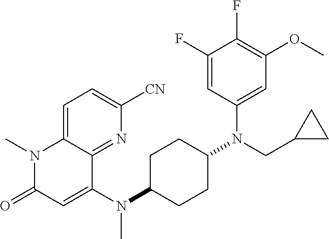 | trans-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2- carbonitrile |
| 96 | 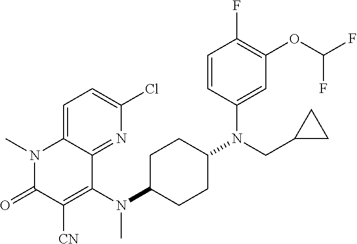 | trans-6-chloro-4-((cyclopropylmethyl)(3- (difluoromethoxy)-4-fluorophenyl)amino)cyclohexyl) (methyl)amino)-1-methyl-2-oxo-1,2-dihydro-1,5- naphthyridine-3-carbonitrile |
| 97 | 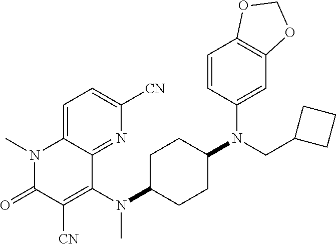 | cis-8-4-(benzo[d][1,3]dioxol-5-yl(cyclobutylmethyl) amino)cyclohexyl)(methyl)amino)-5-methyl-6-oxo- 5,6-dihydro-1,5-naphthyridine-2,7-dicarbonitrile |
| 98 | 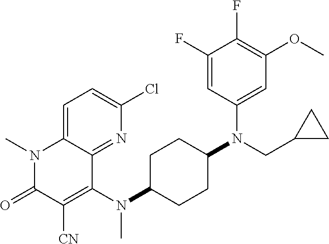 | cis-6-chloro-4-4-((cyclopropylmethyl)(3,4-difluoro-5- methoxyphenyl)amino)cyclohexyl)(methyl)amino)-1- methyl-2-oxo-1,2-dihydro-1,5-naphthyridine-3- carbonitrile |
| 99 | 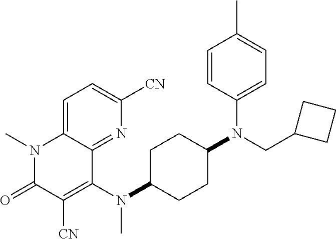 | cis-8-4-((cyclobutylmethyl)(p-tolyl)amino) cyclohexyl)(methyl)amino)-5-methyl-6-oxo-5,6- dihydro-1,5-naphthyridine-2,7-dicarbonitrile |
| 100 | 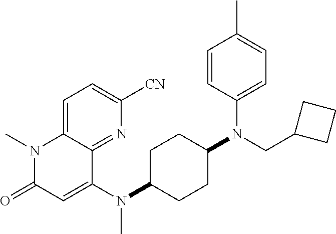 | cis-8-4-((cyclobutylmethyl)(p-tolyl)amino) cyclohexyl)(methyl)amino)-5-methyl-6-oxo-5,6- dihydro-1,5-naphthyridine-2-carbonitrile |
| 101 | 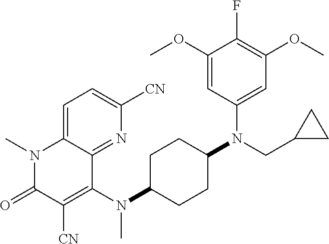 | cis-8-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2,7- dicarbonitrile |
| 102 | 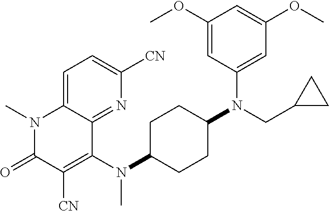 | cis-8-4-((cyclopropylmethyl)(3,5-dimethoxyphenyl) amino)cyclohexyl)(methyl)amino)-5-methyl-6-oxo- 5,6-dihydro-1,5-naphthyridine-2,7-dicarbonitrile |
| 103 | 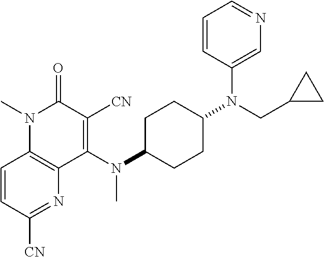 | trans-8-((4-((cyclopropylmethyl)(pyridin-3-yl)amino) cyclohexyl)(methyl)amino)-5-methyl-6-oxo-5,6- dihydro-1,5-naphthyridine-2,7-dicarbonitrile |
| 104 | 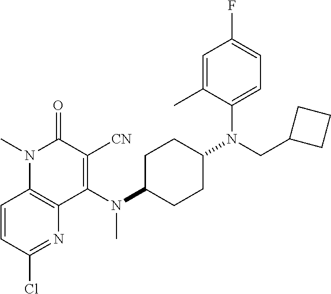 | trans-6-chloro-4-((4-((cyclobutylmethyl)(4-fluoro-2- methylphenyl)amino)cyclohexyl)(methyl)amino)-1- methyl-2-oxo-1,2-dihydro-1,5-naphthyridine-3- carbonitrile |
| 105 | 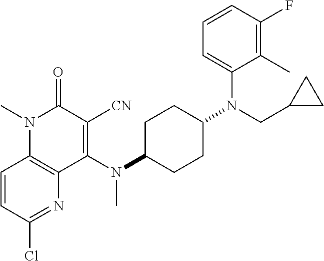 | trans-6-chloro-4-((4-((cyclopropylmethyl)(3-fluoro- 2-methylphenyl)amino)cyclohexyl)(methyl)amino)-1- methyl-2-oxo-1,2-dihydro-1,5-naphthyridine-3- carbonitrile |
| 106 | 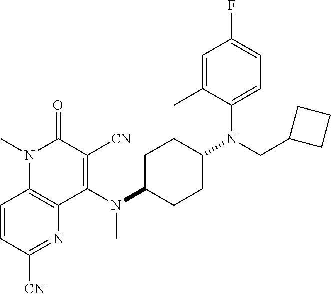 | trans-8-((4-((cyclobutylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 107 | 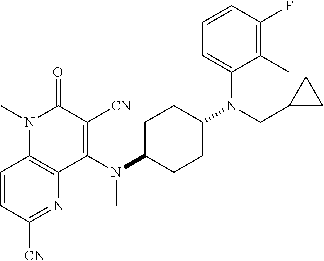 | trans-8-((4-((cyclopropylmethyl)(3-fluoro-2- methylphenyl)amino)cyclohexyl)(methyl)amino)-5- methyl-6-oxo-5,6-dihydro-1,5-naphthyridine-2,7- dicarbonitrile |
| 108 | 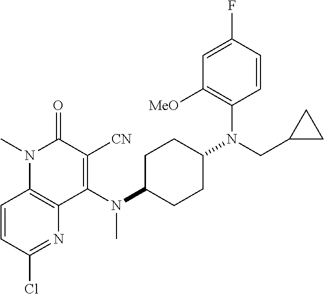 | trans-6-chloro-4-((-4-((cyclopropylmethyl)(4-fluoro- 2-methoxyphenyl)amino)cyclohexyl)(methyl)amino)- 1-methyl-2-oxo-1,2-dihydro-1,5-naphthyridine-3- carbonitrile |
| 109 | 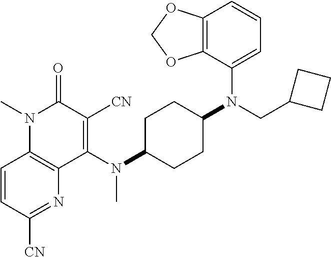 | cis-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2,7-dicarbonitrile |
| 110 | 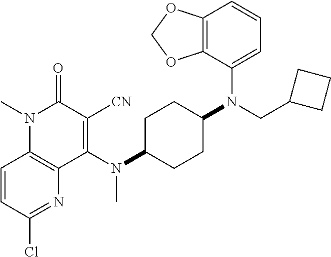 | cis-4-((-4-(benzo[d][1,3]dioxol-4- yl(cyclobutylmethyl)amino)cyclohexyl)(methyl) amino)-6-chloro-1-methyl-2-oxo-1,2-dihydro-1,5- naphthyridine-3-carbonitrile |
| 111 | 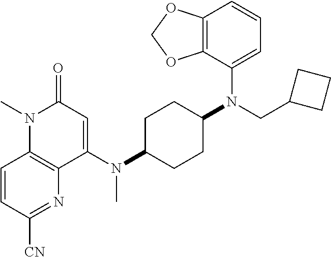 | cis-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2-carbonitrile |
| 112 | 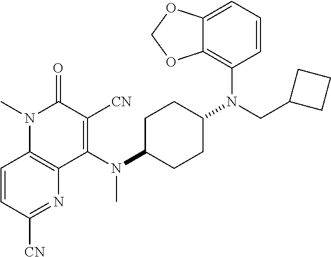 | trans-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2,7-dicarbonitrile |
| 113 | 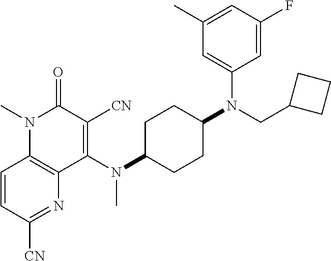 | cis-8-((-4-((cyclobutylmethyl)(3-fluoro-5- methylphenyl)amino)cyclohexyl)(methyl)amino)-5- methyl-6-oxo-5,6-dihydro-1,5-naphthyridine-2,7- dicarbonitrile |
| 114 | 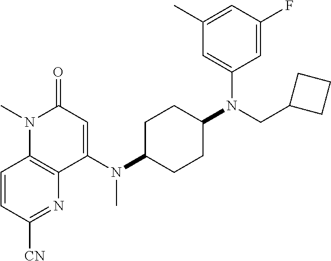 | cis-8-((-4-((cyclobutylmethyl)(3-fluoro-5- methylphenyl)amino)cyclohexyl)(methyl)amino)-5- methyl-6-oxo-5,6-dihydro-1,5-naphthyridine-2- carbonitrile |
| 115 | 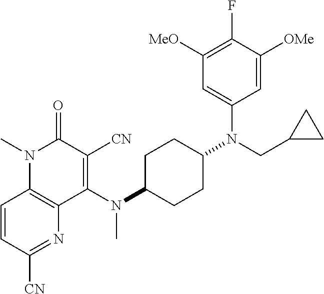 | trans-8-((-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2,7- dicarbonitrile |
| 116 | 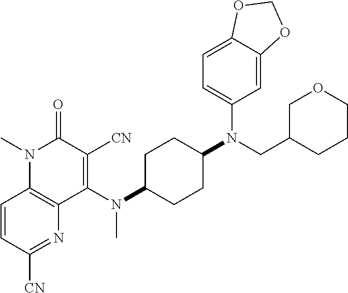 | cis-8-((-4-(benzo[d][1,3]dioxol-5-yl((tetrahydro-2H- pyran-3-yl)methyl)amino)cyclohexyl)(methyl) amino)-5-methyl-6-oxo-5,6-dihydro-1,5- naphthyridine-2,7-dicarbonitrile |
| 117 | 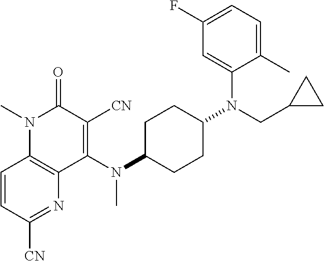 | trans-8-((4-((cyclopropylmethyl)(5-fluoro-2- methylphenyl)amino)cyclohexyl)(methyl)amino)-5- methyl-6-oxo-5,6-dihydro-1,5-naphthyridine-2,7- dicarbonitrile |
| 118 | 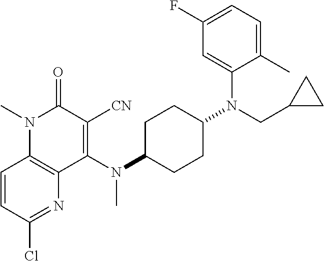 | trans-6-chloro-4-((4-((cyclopropylmethyl)(5-fluoro- 2-methylphenyl)amino)cyclohexyl)(methyl)amino)-1- methyl-2-oxo-1,2-dihydro-1,5-naphthyridine-3- carbonitrile |
| 119 | 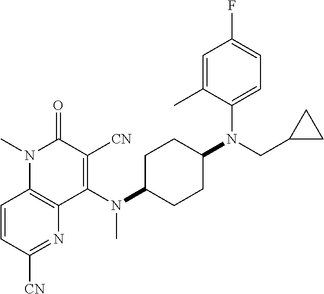 | cis-8-((4-((cyclopropylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 120 | 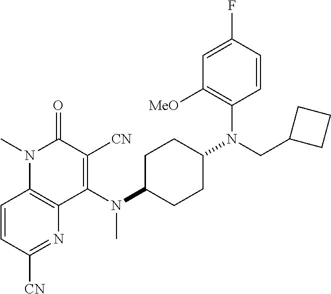 | trans-8-((-4-((cyclobutylmethyl)(4-fluoro-2- methoxyphenyl)amino)cyclohexyl)(methyl)amino)-5- methyl-6-oxo-5,6-dihydro-1,5-naphthyridine-2,7- dicarbonitrile |
| 121 | 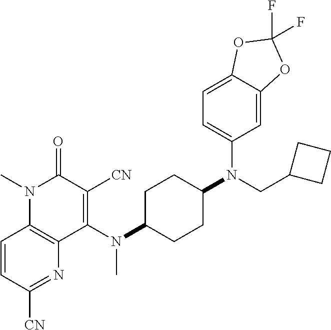 | cis-8-((-4-((cyclobutylmethyl)(2,2-difluorobenzo[d] [1,3]dioxol-5-yl)amino)cyclohexyl)(methyl)amino)- 5-methyl-6-oxo-5,6-dihydro-1,5-naphthyridine-2,7- dicarbonitrile |
| 122 | 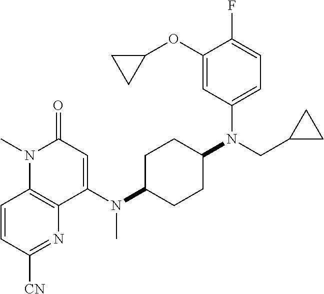 | cis-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2-carbonitrile |
| 123 | 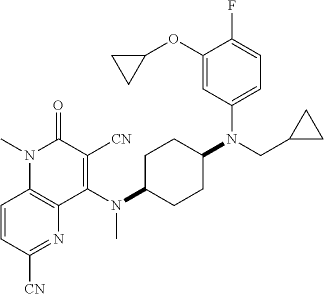 | cis-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2,7-dicarbonitrile |
| 124 | 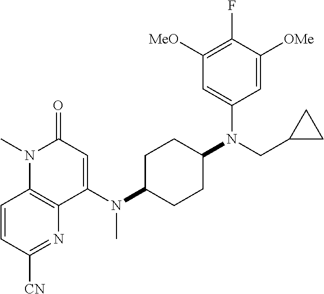 | cis-8-((-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2- carbonitrile |
| 125 | 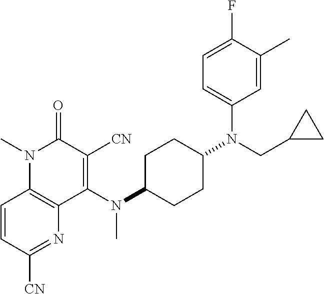 | trans-8-((4-((cyclopropylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 126 | 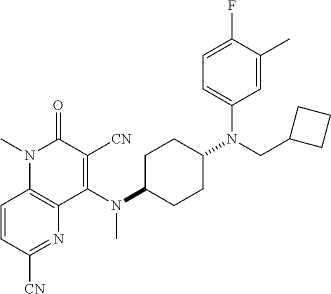 | trans-8-((-4-((cyclobutylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 127 | 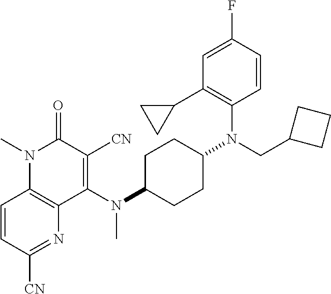 | trans-8-((-4-((cyclobutylmethyl)(2-cyclopropyl-4- fluorophenyl)amino)cyclohexyl)(methyl)amino)-5- methyl-6-oxo-5,6-dihydro-1,5-naphthyridine-2,7- dicarbonitrile |
| 128 | 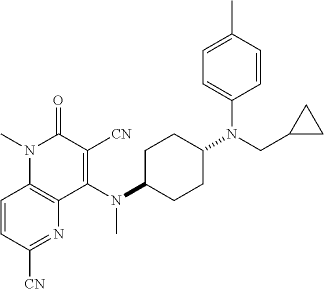 | trans-8-((-4-((cyclopropylmethyl)(p-tolyl)amino) cyclohexyl)(methyl)amino)-5-methyl-6-oxo-5,6- dihydro-1,5-naphthyridine-2,7-dicarbonitrile |
| 129 | 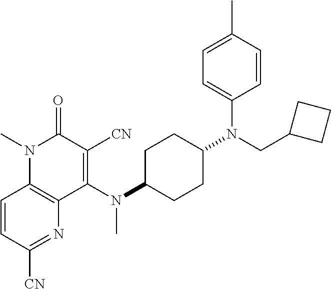 | trans-8-((-4-((cyclobutylmethyl)(p-tolyl)amino) cyclohexyl)(methyl)amino)-5-methyl-6-oxo-5,6- dihydro-1,5-naphthyridine-2,7-dicarbonitrile |
| 130 | 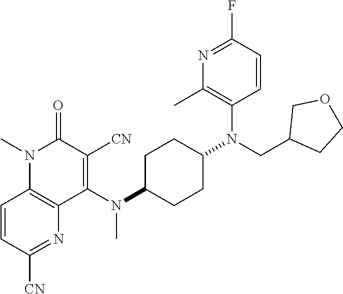 | trans-8-((-4-((6-fluoro-2-methylpyridin-3-yl) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 131 | 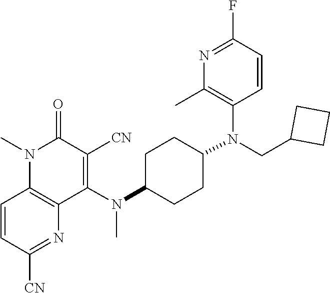 [SMILES: Cc1nc(F)ccc1N(CC1CCC1)[C@H]1CC[C@H](N(C)c2c(C#N)c(=O)n(C)c3ccc(C#N)nc23)CC1] | trans-8-((-4-((cyclobutylmethyl)(6-fluoro-2- methylpyridin-3-yl)amino)cyclohexyl)(methyl) amino)-5-methyl-6-oxo-5,6-dihydro-1,5- naphthyridine-2,7-dicarbonitrile |
| 132 | 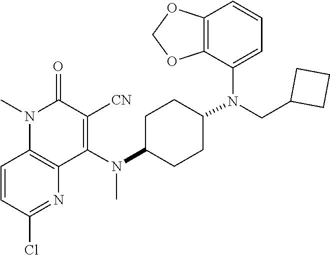 [SMILES: Cn1c(=O)c(C#N)c(N(C)[C@H]2CC[C@H](N(CC3CCC3)c3cccc4c3OCO4)CC2)c2nc(Cl)ccc21] | trans-4-((-4-(benzo[d][1,3]dioxol-4- yl(cyclobutylmethyl)amino)cyclohexyl)(methyl) amino)-6-chloro-1-methyl-2-oxo-1,2-dihydro-1,5- naphthyridine-3-carbonitrile |
| 133 | 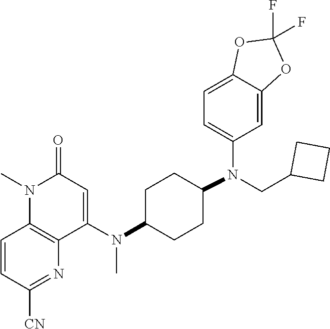 [SMILES: Cn1c(=O)cc(N(C)[C@H]2CC[C@@H](N(CC3CCC3)c3ccc4c(c3)OC(F)(F)O4)CC2)c2nc(C#N)ccc21] | cis-8-((-4-((cyclobutylmethyl)(2,2-difluorobenzo[d] [1,3]dioxol-5-yl)amino)cyclohexyl)(methyl)amino)- 5-methyl-6-oxo-5,6-dihydro-1,5-naphthyridine-2- carbonitrile |
| 134 | 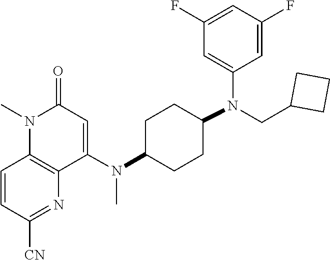 [SMILES: Cn1c(=O)cc(N(C)[C@H]2CC[C@@H](N(CC3CCC3)c3cc(F)cc(F)c3)CC2)c2nc(C#N)ccc21] | cis-8-((-4-((cyclobutylmethyl)(3,5-difluorophenyl) amino)cyclohexyl)(methyl)amino)-5-methyl-6-oxo- 5,6-dihydro-1,5-naphthyridine-2-carbonitrile |
| 135 | 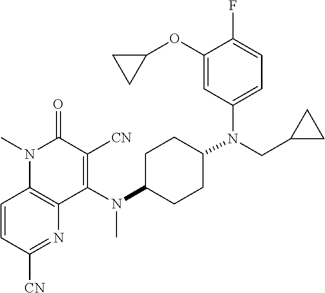 [SMILES: Cn1c(=O)c(C#N)c(N(C)[C@H]2CC[C@H](N(CC3CC3)c3ccc(F)c(OC4CC4)c3)CC2)c2nc(C#N)ccc21] | trans-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2,7-dicarbonitrile |
| 136 | 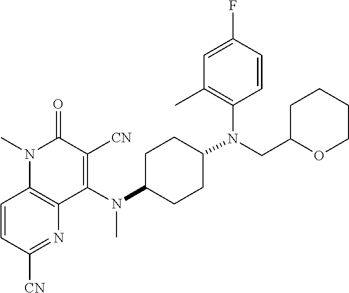 [SMILES: Cc1cc(F)ccc1N(CC1CCCCO1)[C@H]1CC[C@H](N(C)c2c(C#N)c(=O)n(C)c3ccc(C#N)nc23)CC1] | trans-8-((-4-((4-fluoro-2-methylphenyl)((tetrahydro- 2H-pyran-2-yl)methyl)amino)cyclohexyl)(methyl) amino)-5-methyl-6-oxo-5,6-dihydro-1,5- naphthyridine-2,7-dicarbonitrile |
| 137 | 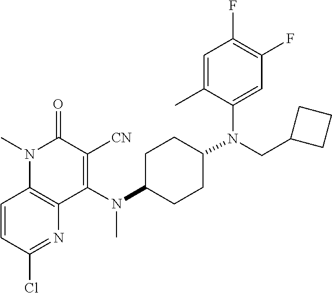 [SMILES: Cc1cc(F)c(F)cc1N(CC1CCC1)[C@H]1CC[C@H](N(C)c2c(C#N)c(=O)n(C)c3ccc(Cl)nc23)CC1] | trans-6-chloro-4-((4-((cyclobutylmethyl)(4,5- difluoro-2-methylphenyl)amino)cyclohexyl)(methyl) amino)-1-methyl-2-oxo-1,2-dihydro-1,5- naphthyridine-3-carbonitrile |
| 138 | 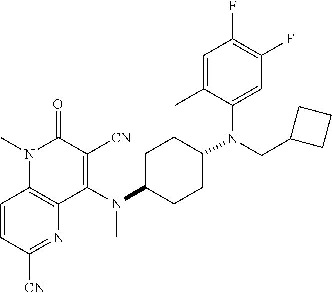 [SMILES: Cc1cc(F)c(F)cc1N(CC1CCC1)[C@H]1CC[C@H](N(C)c2c(C#N)c(=O)n(C)c3ccc(C#N)nc23)CC1] | trans-8-((4-((cyclobutylmethyl)(4,5-difluoro-2- methylphenyl)amino)cyclohexyl)(methyl)amino)-5- methyl-6-oxo-5,6-dihydro-1,5-naphthyridine-2,7- dicarbonitrile |
| 139 | 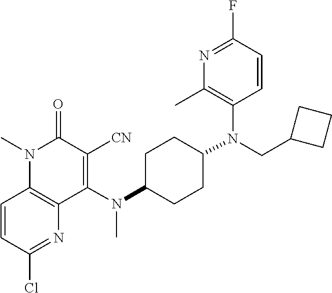 [SMILES: Cc1nc(F)ccc1N(CC1CCC1)[C@H]1CC[C@H](N(C)c2c(C#N)c(=O)n(C)c3ccc(Cl)nc23)CC1] | trans-6-chloro-4-((-4-((cyclobutylmethyl)(6-fluoro-2- methylpyridin-3-yl)amino)cyclohexyl)(methyl) amino)-1-methyl-2-oxo-1,2-dihydro-1,5- naphthyridine-3-carbonitrile |
| 140 | 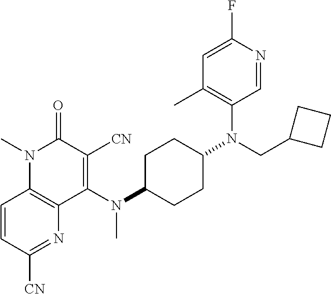 | trans-8-((-4-((cyclobutylmethyl)(6-fluoro-4- methylpyridin-3-yl)amino)cyclohexyl)(methyl) amino)-5-methyl-6-oxo-5,6-dihydro-1,5- naphthyridine-2,7-dicarbonitrile |
| 14 | 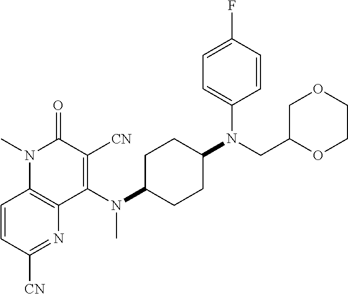 | cis-8-((-4-(((1,4-dioxan-2-yl)methyl)(4-fluorophenyl) amino)cyclohexyl)(methyl)amino)-5-methyl-6-oxo- 5,6-dihydro-1,5-naphthyridine-2,7-dicarbonitrile |
| 142 | 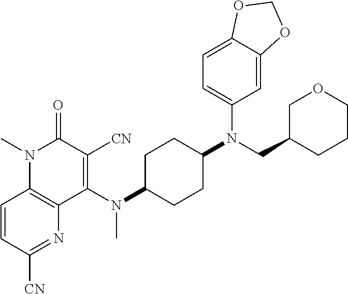 | cis-8-((-4-(benzo[d][1,3]dioxol-5-yl(((S)-tetrahydro- 2H-pyran-3-yl)methyl)amino)cyclohexyl)(methyl) amino)-5-methyl-6-oxo-5,6-dihydro-1,5- naphthyridine-2,7-dicarbonitrile |
| 143 | 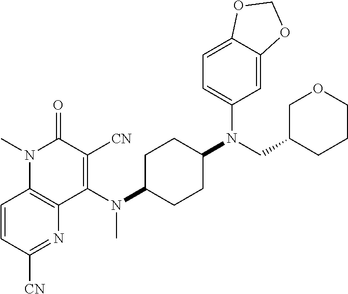 | cis-8-((-4-(benzo[d][1,3]dioxol-5-yl(((R)-tetrahydro- 2H-pyran-3-yl)methyl)amino)cyclohexyl)(methyl) amino)-5-methyl-6-oxo-5,6-dihydro-1,5- naphthyridine-2,7-dicarbonitrile |
| 144 | 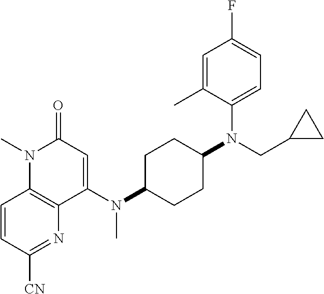 | cis-8-((4-((cyclopropylmethyl)(4-fluoro-2- methylphenyl)amino)cyclohexyl)(methyl)amino)-5- methyl-6-oxo-5,6-dihydro-1,5-naphthyridine-2- carbonitrile |
| 145 | 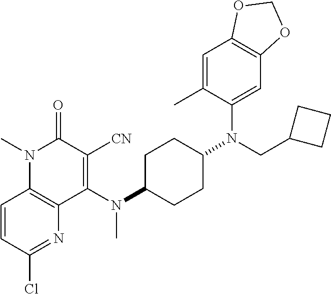 | trans-6-chloro-4-((4-((cyclobutylmethyl)(6- methylbenzo[d][1,3]dioxol-5-yl)amino)cyclohexyl) (methyl)amino)-1-methyl-2-oxo-1,2-dihydro-1,5- naphthyridine-3-carbonitrile |
| 146 | 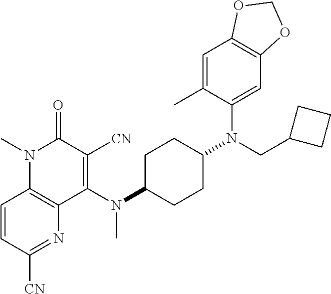 | trans-8-((4-((cyclobutylmethyl)(6-methylbenzo[d] [1,3]dioxol-5-yl)amino)cyclohexyl)(methyl)amino)- 5-methyl-6-oxo-5,6-dihydro-1,5-naphthyridine-2,7- dicarbonitrile |
| 147 | 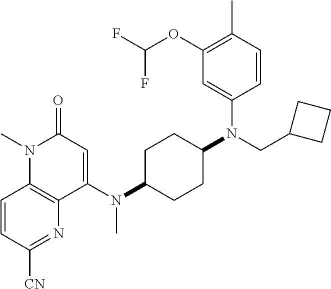 | cis-8-((-4-((cyclobutylmethyl)(3-(difluoromethoxy)- 4-methylphenyl)amino)cyclohexyl)(methyl)amino)-5- methyl-6-oxo-5,6-dihydro-1,5-naphthyridine-2- carbonitrile |
| 148 | 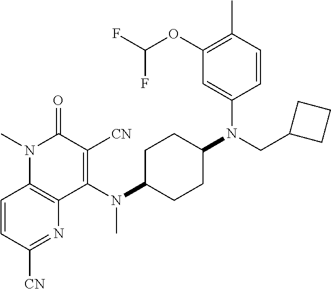 | cis-8-((-4-((cyclobutylmethyl)(3-(difluoromethoxy)- 4-methylphenyl)amino)cyclohexyl)(methyl)amino)-5- methyl-6-oxo-5,6-dihydro-1,5-naphthyridine-2,7- dicarbonitrile |
| 149 | 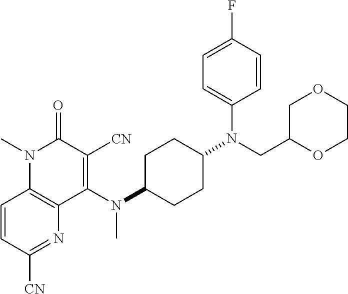 | trans-8-((-4-(((1,4-dioxan-2-yl)methyl)(4- fluorophenyl)amino)cyclohexyl)(methyl)amino)-5- methyl-6-oxo-5,6-dihydro-1,5-naphthyridine-2,7- dicarbonitrile |
| 150 | 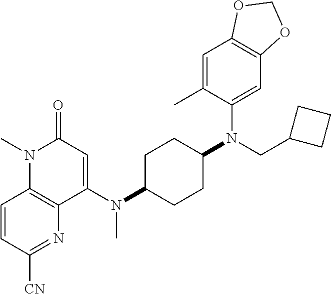 | cis-8-((4-((cyclobutylmethyl)(6-methylbenzo[d][1,3] dioxol-5-yl)amino)cyclohexyl)(methyl)amino)-5- methyl-6-oxo-5,6-dihydro-1,5-naphthyridine-2- carbonitrile |
| 151 | 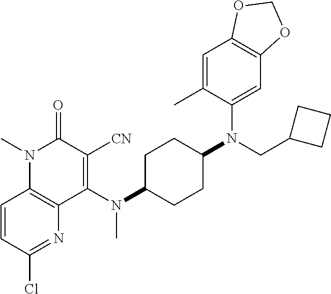 | cis-6-chloro-4-((4-((cyclobutylmethyl)(6- methylbenzo[d][1,3]dioxol-5-yl)amino)cyclohexyl) (methyl)amino)-1-methyl-2-oxo-1,2-dihydro-1,5- naphthyridine-3-carbonitrile |
| 152 | 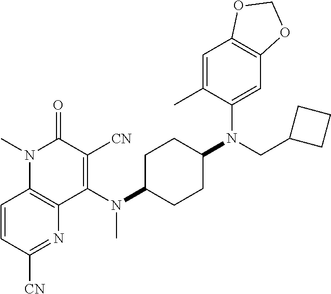 | cis-8-((4-((cyclobutylmethyl)(6-methylbenzo[d][1,3] dioxol-5-yl)amino)cyclohexyl)(methyl)amino)-5- methyl-6-oxo-5,6-dihydro-1,5-naphthyridine-2,7- dicarbonitrile |
| 153 | 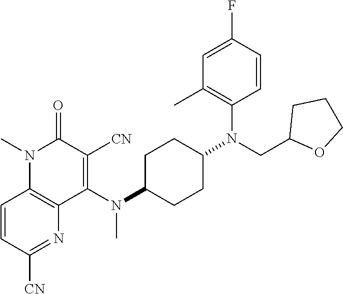 | cis-8-((-4-((4-fluoro-2-methylphenyl) ((tetrahydrofuran-2-yl)methyl)amino)cyclohexyl) (methyl)amino)-5-methyl-6-oxo-5,6-dihydro-1,5- naphthyridine-2,7-dicarbonitrile |
| 154 | 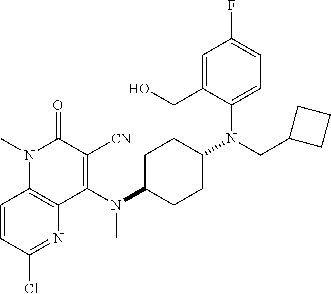 | trans-6-chloro-4-((4-((cyclobutylmethyl)(4-fluoro-2- (hydroxymethyl)phenyl)amino)cyclohexyl)(methyl) amino)-1-methyl-2-oxo-1,2-dihydro-1,5- naphthyridine-3-carbonitrile |
| 155 | 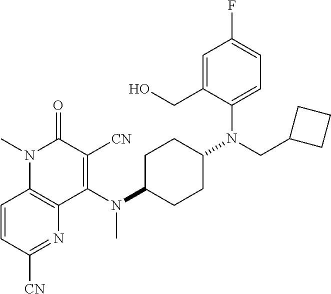 | trans-8-((4-((cyclobutylmethyl)(4-fluoro-2- (hydroxymethyl)phenyl)amino)cyclohexyl)(methyl) amino)-5-methyl-6-oxo-5,6-dihydro-1,5- naphthyridine-2,7-dicarbonitrile |
| 156 | 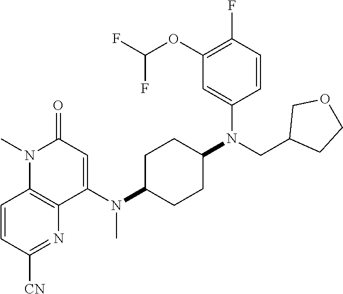 | cis-8-((-4-((3-(difluoromethoxy)-4-fluorophenyl) ((tetrahydrofuran-3-yl)methyl)amino)cyclohexyl) (methyl)amino)-5-methyl-6-oxo-5,6-dihydro-1,5- naphthyridine-2-carbonitrile |
| 157 | 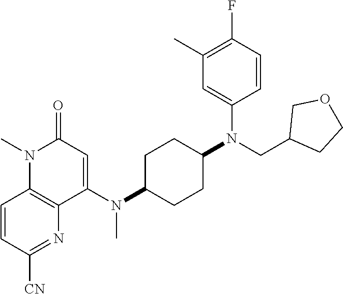 | cis-8-((-4-((4-fluoro-3-methylphenyl) ((tetrahydrofuran-3-yl)methyl)amino)cyclohexyl) (methyl)amino)-5-methyl-6-oxo-5,6-dihydro-1,5- naphthyridine-2-carbonitrile |
| 158 | 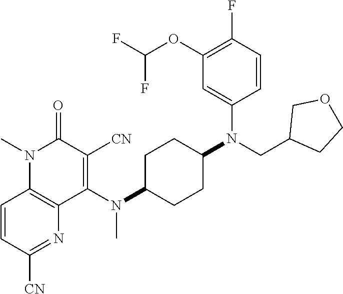 | cis-8-((-4-((3-(difluoromethoxy)-4-fluorophenyl) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 159 | 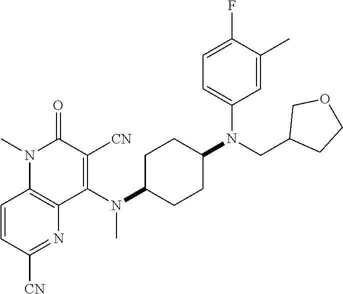 | cis-8-((-4-((4-fluoro-3-methylphenyl) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 160 | 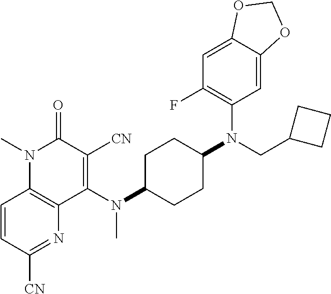 | cis-8-((-4-((cyclobutylmethyl)(6-fluorobenzo[d][1,3] dioxol-5-yl)amino)cyclohexyl)(methyl)amino)-5- methyl-6-oxo-5,6-dihydro-1,5-naphthyridine-2,7- dicarbonitrile |
| 161 | 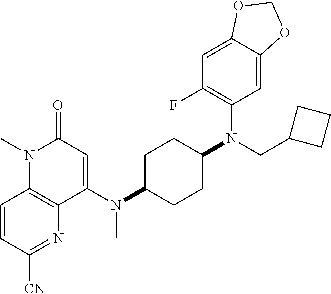 | cis-8-((-4-((cyclobutylmethyl)(6-fluorobenzo[d][1,3] dioxol-5-yl)amino)cyclohexyl)(methyl)amino)-5- methyl-6-oxo-5,6-dihydro-1,5-naphthyridine-2- carbonitrile |
| 162 | 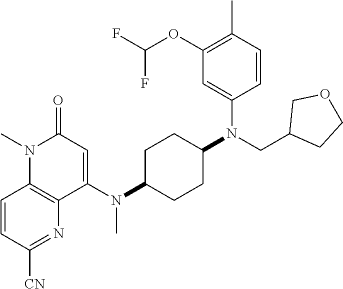 | cis-8-((-4-((3-(difluoromethoxy)-4-methylphenyl) ((tetrahydrofuran-3-yl)methyl)amino)cyclohexyl) (methyl)amino)-5-methyl-6-oxo-5,6-dihydro-1,5- naphthyridine-2-carbonitrile |
| 163 | 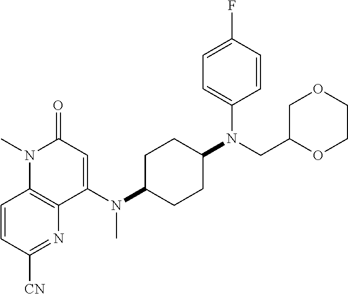 | cis-8-((-4-(((1,4-dioxan-2-yl)methyl)(4-fluorophenyl) amino)cyclohexyl)(methyl)amino)-5-methyl-6-oxo- 5,6-dihydro-1,5-naphthyridine-2-carbonitrile |
| 164 | 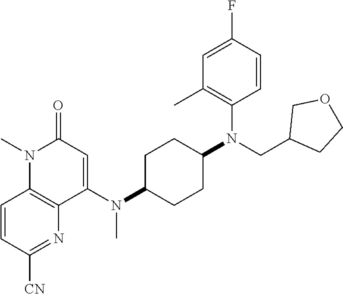 | cis-8-((-4-((4-fluoro-2-methylphenyl) ((tetrahydrofuran-3-yl)methyl)amino)cyclohexyl) (methyl)amino)-5-methyl-6-oxo-5,6-dihydro-1,5- naphthyridine-2-carbonitrile |
| 165 | 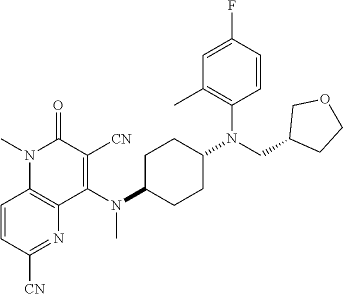 | trans-8-((-4-((4-fluoro-2-methylphenyl)(((R)- tetrahydrofuran-3-yl)methyl)amino)cyclohexyl) (methyl)amino)-5-methyl-6-oxo-5,6-dihydro-1,5- naphthyridine-2,7-dicarbonitrile |
| 166 | 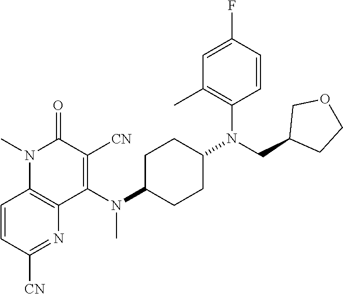 | trans-8-((-4-((4-fluoro-2-methylphenyl)(((S)- tetrahydrofuran-3-yl)methyl)amino)cyclohexyl) (methyl)amino)-5-methyl-6-oxo-5,6-dihydro-1,5- naphthyridine-2,7-dicarbonitrile |
| 167 | 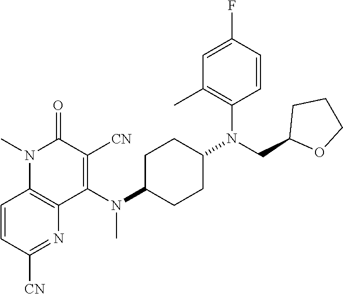 | trans-8-((-4-((4-fluoro-2-methylphenyl)(((R)- tetrahydrofuran-2-yl)methyl)amino)cyclohexyl) (methyl)amino)-5-methyl-6-oxo-5,6-dihydro-1,5- naphthyridine-2,7-dicarbonitrile |
| 168 | 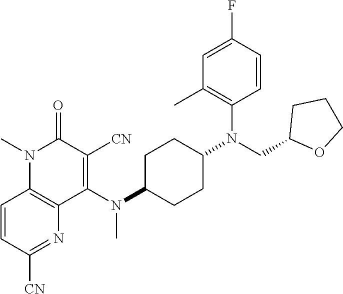 | trans-8-((-4-((4-fluoro-2-methylphenyl)(((S)- tetrahydrofuran-2-yl)methyl)amino)cyclohexyl) (methyl)amino)-5-methyl-6-oxo-5,6-dihydro-1,5- naphthyridine-2,7-dicarbonitrile |
| 169 | 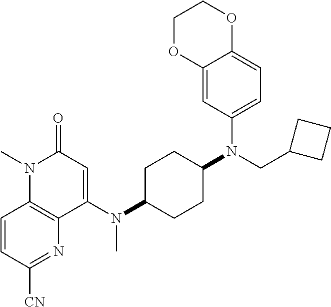 | cis-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2- carbonitrile |
| 170 | 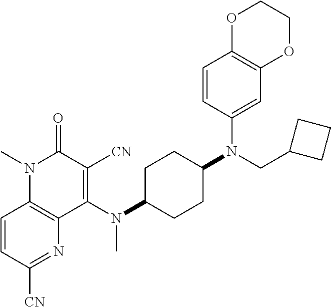 | cis-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2,7- dicarbonitrile |
| 171 | 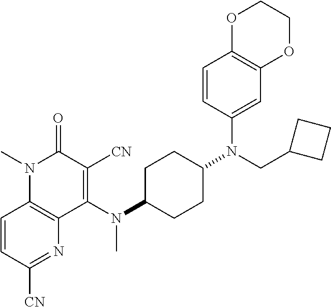 | trans-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2,7- dicarbonitrile |
| 172 | 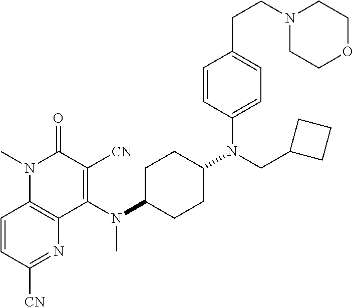 | trans-8-((4-((cyclobutylmethyl)(4-(2- morpholinoethyl)phenyl)amino)cyclohexyl)(methyl) amino)-5-methyl-6-oxo-5,6-dihydro-1,5- naphthyridine-2,7-dicarbonitrile |
| 173 | 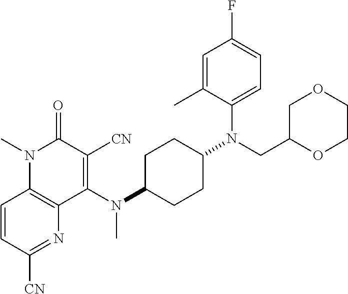 | trans-8-((4-(((1,4-dioxan-2-yl)methyl)(4-fluoro-2- methylphenyl)amino)cyclohexyl)(methyl)amino)-5- methyl-6-oxo-5,6-dihydro-1,5-naphthyridine-2,7- dicarbonitrile |
| 174 | 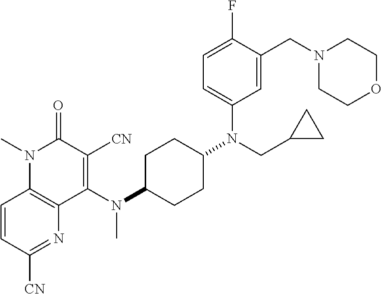 | trans-8-((-4-((cyclopropylmethyl)(4-fluoro-3- (morpholinomethyl)phenyl)amino)cyclohexyl) (methyl)amino)-5-methyl-6-oxo-5,6-dihydro-1,5- naphthyridine-2,7-dicarbonitrile |
| 175 | 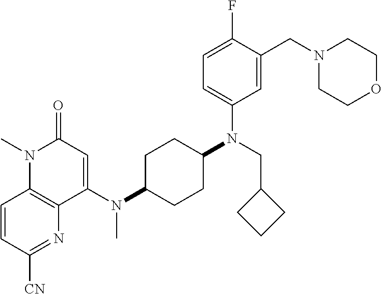 | cis-8-((-4-((cyclobutylmethyl)(4-fluoro-3- (morpholinomethyl)phenyl)amino)cyclohexyl) (methyl)amino)-5-methyl-6-oxo-5,6-dihydro-1,5- naphthyridine-2-carbonitrile |
| 176 | 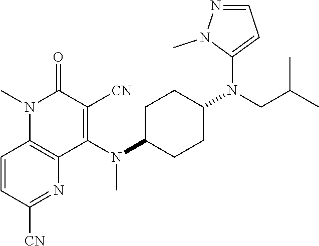 | trans-8-((-4-(isobutyl(1-methyl-1H-pyrazol-5-yl) amino)cyclohexyl)(methyl)amino)-5-methyl-6-oxo- 5,6-dihydro-1,5-naphthyridine-2,7-dicarbonitrile |
| 177 | 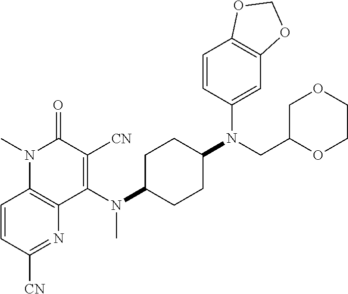 | cis-8-((4-(((1,4-dioxan-2-yl)methyl)(benzo[d][1,3] dioxol-5-yl)amino)cyclohexyl)(methyl)amino)-5- methyl-6-oxo-5,6-dihydro-1,5-naphthyridine-2,7- dicarbonitrile |
| 178 | 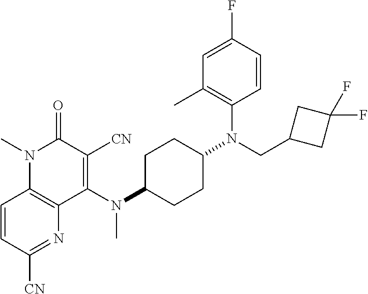 | trans-8-((-4-(((3,3-difluorocyclobutyl)methyl)(4- fluoro-2-methylphenyl)amino)cyclohexyl)(methyl) amino)-5-methyl-6-oxo-5,6-dihydro-1,5- naphthyridine-2,7-dicarbonitrile |
| 179 | 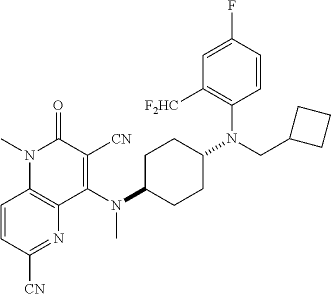 | trans-8-((-4-((cyclobutylmethyl)(2-(difluoromethyl)- 4-fluorophenyl)amino)cyclohexyl)(methyl)amino)-5- methyl-6-oxo-5,6-dihydro-1,5-naphthyridine-2,7- dicarbonitrile |
| 180 | 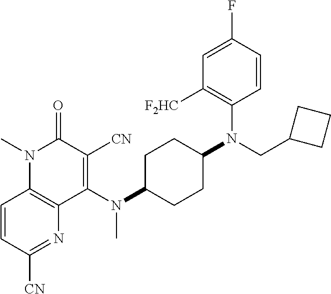 | cis-8-((-4-((cyclobutylmethyl)(2-(difluoromethyl)-4- fluorophenyl)amino)cyclohexyl)(methyl)amino)-5- methyl-6-oxo-5,6-dihydro-1,5-naphthyridine-2,7- dicarbonitrile |
| 181 | 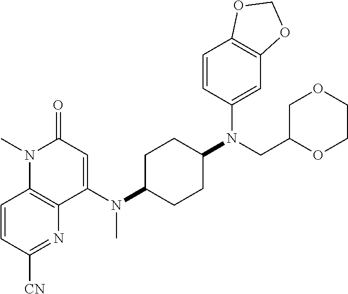 | cis-8-((4-(((1,4-dioxan-2-yl)methyl)(benzo[d][1,3] dioxol-5-yl)amino)cyclohexyl)(methyl)amino)-5- methyl-6-oxo-5,6-dihydro-1,5-naphthyridine-2- carbonitrile |
| 182 | 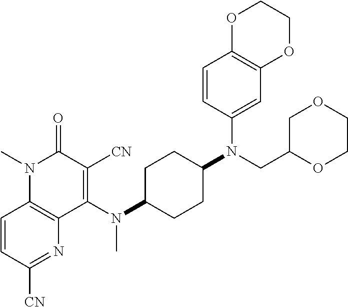 | cis-8-((4-(((1,4-dioxan-2-yl)methyl)(2,3- dihydrobenzo[b][1,4]dioxin-6-yl)amino)cyclohexyl) (methyl)amino)-5-methyl-6-oxo-5,6-dihydro-1,5- naphthyridine-2,7-dicarbonitrile |
| 183 | 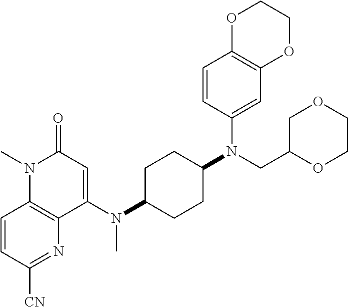 | cis-8-((4-(((1,4-dioxan-2-yl)methyl)(2,3- dihydrobenzo[b][1,4]dioxin-6-yl)amino)cyclohexyl) (methyl)amino)-5-methyl-6-oxo-5,6-dihydro-1,5- naphthyridine-2-carbonitrile |
| 184 | 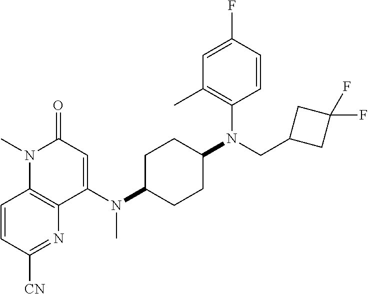 | cis-8-((-4-(((3,3-difluorocyclobutyl)methyl)(4-fluoro- 2-methylphenyl)amino)cyclohexyl)(methyl)amino)-5- methyl-6-oxo-5,6-dihydro-1,5-naphthyridine-2- carbonitrile |
| 185 | 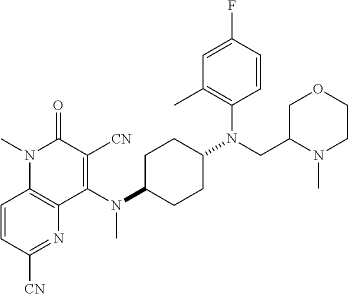 | trans-8-((-4-((4-fluoro-2-methylphenyl)((4- methylmorpholin-3-yl)methyl)amino)cyclohexyl) (methyl)amino)-5-methyl-6-oxo-5,6-dihydro-1,5- naphthyridine-2,7-dicarbonitrile |
| 186 | 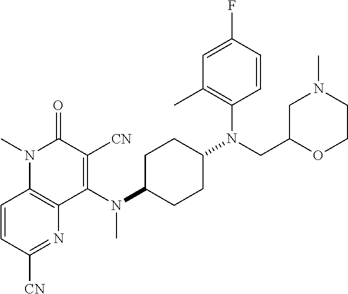 | trans-8-((-4-((4-fluoro-2-methylphenyl)((4- methylmorpholinium-2-yl)methyl)amino)cyclohexyl) (methyl)amino)-5-methyl-6-oxo-5,6-dihydro-1,5- naphthyridine-2,7-dicarbonitrile trifluoroacetate |
| 187 | 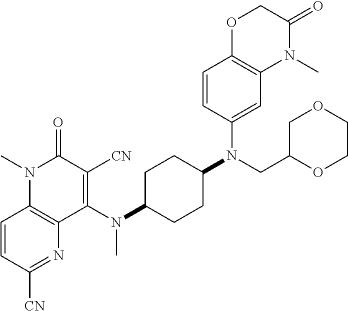 | cis-8-((4-(((1,4-dioxan-2-yl)methyl)(4-methyl-3-oxo- 3,4-dihydro-2H-benzo[b][1,4]oxazin-6-yl)amino) cyclohexyl)(methyl)amino)-5-methyl-6-oxo-5,6- dihydro-1,5-naphthyridine-2,7-dicarbonitrile |
| 188 | 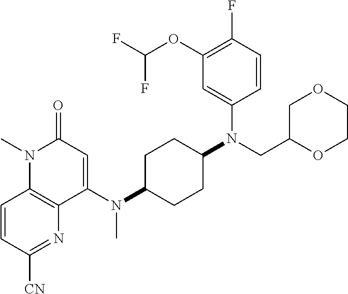 | cis-8-((-4-(((1,4-dioxan-2-yl)methyl)(3- (difluoromethoxy)-4-fluorophenyl)amino)cyclohexyl) (methyl)amino)-5-methyl-6-oxo-5,6-dihydro-1,5- naphthyridine-2-carbonitrile |
| 189 | 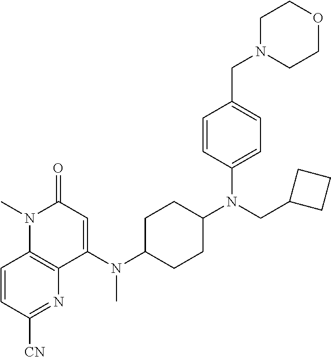 [SMILES: CN(c1cc(=O)n(C)c2ccc(C#N)nc12)C1CCC(N(CC2CCC2)c2ccc(CN3CCOCC3)cc2)CC1] | (cis or trans)-8-((4-((cyclobutylmethyl)(4- (morpholinomethyl)phenyl)amino)cyclohexyl) (methyl)amino)-5-methyl-6-oxo-5,6-dihydro-1,5- naphthyridine-2-carbonitrile |
| 190 | 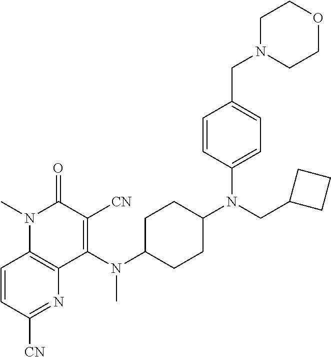 [SMILES: CN(c1c(C#N)c(=O)n(C)c2ccc(C#N)nc12)C1CCC(N(CC2CCC2)c2ccc(CN3CCOCC3)cc2)CC1] | (cis or trans)-8-((4-((cyclobutylmethyl)(4- (morpholinomethyl)phenyl)amino)cyclohexyl) (methyl)amino)-5-methyl-6-oxo-5,6-dihydro-1,5- naphthyridine-2,7-dicarbonitrile |
| 191 | 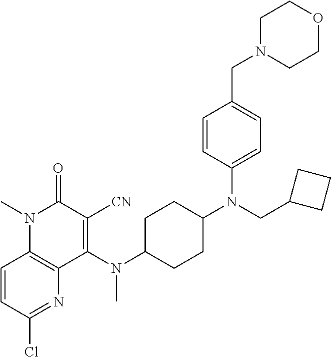 [SMILES: CN(c1c(C#N)c(=O)n(C)c2ccc(Cl)nc12)C1CCC(N(CC2CCC2)c2ccc(CN3CCOCC3)cc2)CC1] | (cis or trans)-6-chloro-4-((4-((cyclobutylmethyl)(4- (morpholinomethyl)phenyl)amino)cyclohexyl) (methyl)amino)-1-methyl-2-oxo-1,2-dihydro-1,5- naphthyridine-3-carbonitrile |
| 192 | 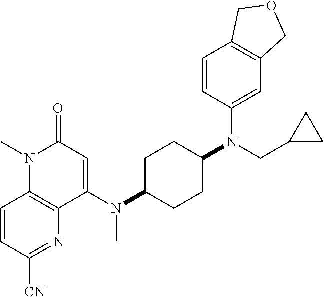 | cis-8-((4-((cyclopropylmethyl)(1,3- dihydroisobenzofuran-5-yl)amino)cyclohexyl) (methyl)amino)-5-methyl-6-oxo-5,6-dihydro-1,5- naphthyridine-2-carbonitrile |
| 193 | 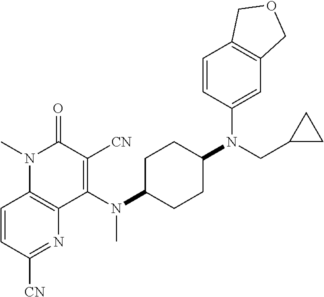 | cis-8-((4-((cyclopropylmethyl)(1,3- dihydroisobenzofuran-5-yl)amino)cyclohexyl) (methyl)amino)-5-methyl-6-oxo-5,6-dihydro-1,5- naphthyridine-2,7-dicarbonitrile |
| 194 | 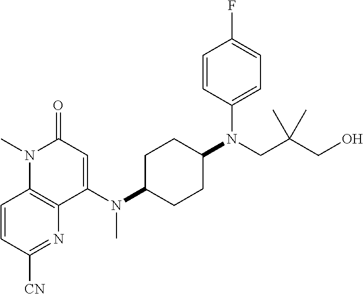 | cis-8-((-4-((4-fluorophenyl)(3-hydroxy-2,2- dimethylpropyl)amino)cyclohexyl)(methyl)amino)-5- methyl-6-oxo-5,6-dihydro-1,5-naphthyridine-2- carbonitrile |
| 195 | 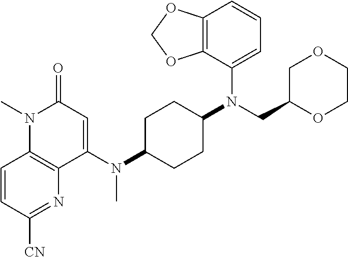 | cis-8-((-4-((((S or R)-1,4-dioxan-2-yl)methyl) (benzo[d][1,3]dioxol-4-yl)amino)cyclohexyl)(methyl) amino)-5-methyl-6-oxo-5,6-dihydro-1,5- naphthyridine-2-carbonitrile |
| 196 | 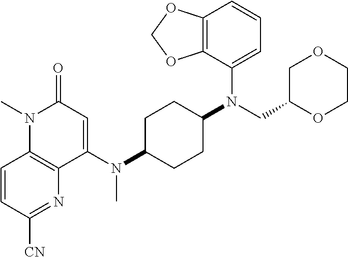 | cis-8-((-4-((((R or S)-1,4-dioxan-2-yl)methyl) (benzo[d][1,3]dioxol-4-yl)amino)cyclohexyl)(methyl) amino)-5-methyl-6-oxo-5,6-dihydro-1,5- naphthyridine-2-carbonitrile |
| 197 | 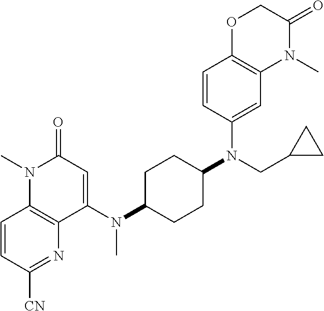 | cis-8-((4-((cyclopropylmethyl)(4-methyl-3-oxo-3,4- dihydro-2H-benzo[b][1,4]oxazin-6-yl)amino) cyclohexyl)(methyl)amino)-5-methyl-6-oxo-5,6- dihydro-1,5-naphthyridine-2-carbonitrile |
| 198 | 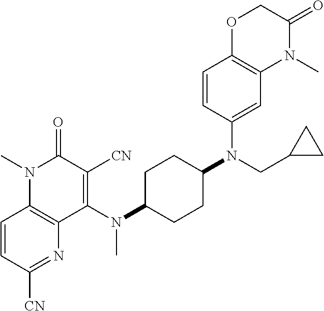 | cis-8-((4-((cyclopropylmethyl)(4-methyl-3-oxo-3,4- dihydro-2H-benzo[b][1,4]oxazin-6-yl)amino) cyclohexyl)(methyl)amino)-5-methyl-6-oxo-5,6- dihydro-1,5-naphthyridine-2,7-dicarbonitrile |
| 199 | 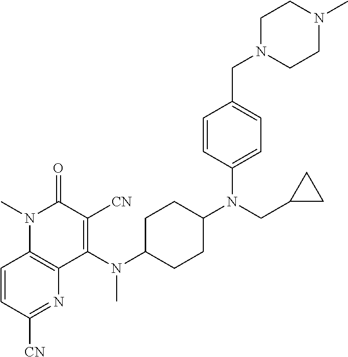 [SMILES: CN1CCN(Cc2ccc(N(CC3CC3)C3CCC(N(C)c4c(C#N)c(=O)n(C)c5ccc(C#N)nc45)CC3)cc2)CC1] | (cis or trans)-8-((4-((cyclopropylmethyl)(4-((4- methylpiperazin-1-yl)methyl)phenyl)amino) cyclohexyl)(methyl)amino)-5-methyl-6-oxo-5,6- dihydro-1,5-naphthyridine-2,7-dicarbonitrile |
| 200 | 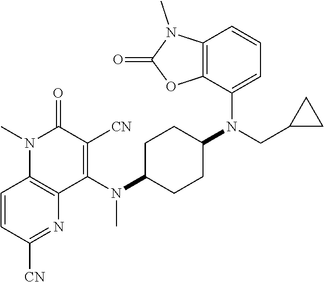 | cis-8-((-4-((cyclopropylmethyl)(3-methyl-2-oxo-2,3- dihydrobenzo[d]oxazol-7-yl)amino)cyclohexyl) (methyl)amino)-5-methyl-6-oxo-5,6-dihydro-1,5- naphthyridine-2,7-dicarbonitrile |
| 201 | 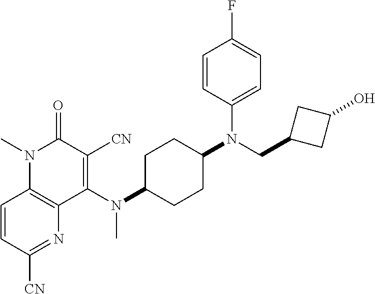 | cis-8-((-4-((4-fluorophenyl)((trans-3- hydroxycyclobutyl)methyl)amino)cyclohexyl) (methyl)amino)-5-methyl-6-oxo-5,6-dihydro-1,5- naphthyridine-2,7-dicarbonitrile |
| 202 | 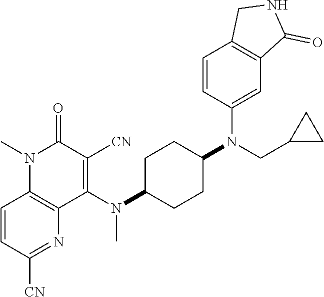 | cis-8-((-4-((cyclopropylmethyl)(3-oxoisoindolin-5-yl) amino)cyclohexyl)(methyl)amino)-5-methyl-6-oxo- 5,6-dihydro-1,5-naphthyridine-2,7-dicarbonitrile |
| 203 | 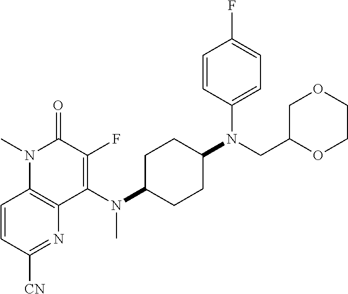 | cis-8-((-4-(((1,4-dioxan-2-yl)methyl)(4-fluorophenyl) amino)cyclohexyl)(methyl)amino)-7-fluoro-5- methyl-6-oxo-5,6-dihydro-1,5-naphthyridine-2- carbonitrile |
| 204 | 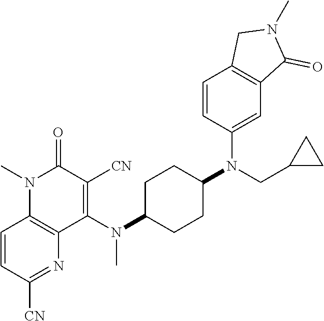 | cis-8-((-4-((cyclopropylmethyl)(2-methyl-3- oxoisoindolin-5-yl)amino)cyclohexyl)(methyl) amino)-5-methyl-6-oxo-5,6-dihydro-1,5- naphthyridine-2,7-dicarbonitrile |
| 205 | 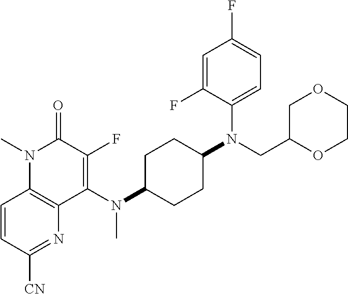 | cis-8-((-4-(((1,4-dioxan-2-yl)methyl)(2,4- difluorophenyl)amino)cyclohexyl)(methyl)amino)-7- fluoro-5-methyl-6-oxo-5,6-dihydro-1,5- naphthyridine-2-carbonitrile |
| 206 | 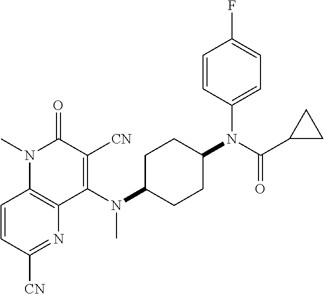 | cis-N-(4-((3,6-dicyano-1-methyl-2-oxo-1,2-dihydro- 1,5-naphthyridin-4-yl)(methyl)amino)cyclohexyl)-N- (4-fluorophenyl)cyclopropanecarboxamide |
| 207 | 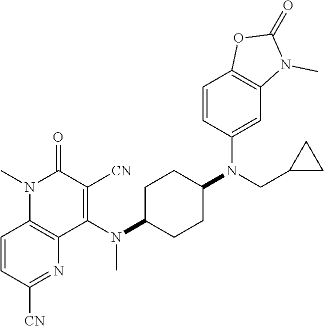 | cis-8-((-4-((cyclopropylmethyl)(3-methyl-2-oxo-2,3- dihydrobenzo[d]oxazol-5-yl)amino)cyclohexyl) (methyl)amino)-5-methyl-6-oxo-5,6-dihydro-1,5- naphthyridine-2,7-dicarbonitrile |
| 208 | 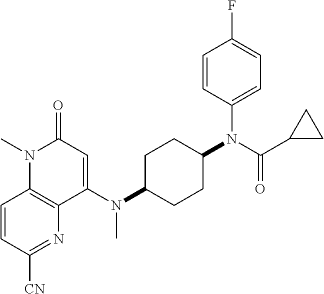 | cis-N-(4-((6-cyano-1-methyl-2-oxo-1,2-dihydro-1,5- naphthyridin-4-yl)(methyl)amino)cyclohexyl)-N-(4- fluorophenyl)cyclopropanecarboxamide |
| 209 | 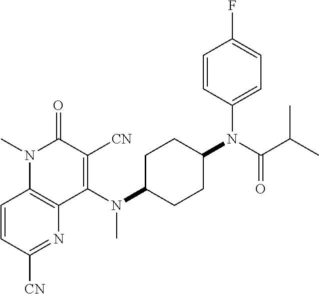 | cis-N-(4-((3,6-dicyano-1-methyl-2-oxo-1,2-dihydro- 1,5-naphthyridin-4-yl)(methyl)amino)cyclohexyl)-N- (4-fluorophenyl)isobutyramide |
| 210 | 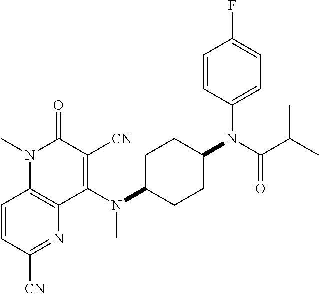 | cis-N-(4-((3,6-dicyano-1-methyl-2-oxo-1,2-dihydro- 1,5-naphthyridin-4-yl)(methyl)amino)cyclohexyl)-N- (4-fluorophenyl)isobutyramide |
| 211 | 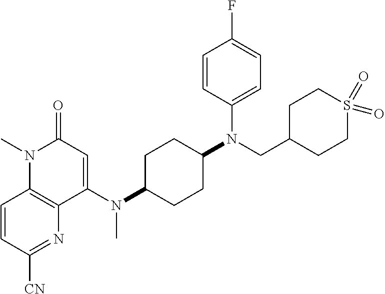 | cis-8-((4-(((1,1-dioxidotetrahydro-2H-thiopyran-4-yl) methyl)(4-fluorophenyl)amino)cyclohexyl)(methyl) amino)-5-methyl-6-oxo-5,6-dihydro-1,5- naphthyridine-2-carbonitrile |
| 212 | 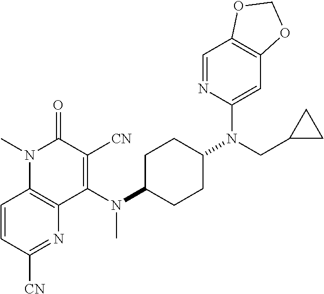 | trans-8-((-4-([1,3]dioxolo[4,5-c]pyridin-6- yl(cyclopropylmethyl)amino)cyclohexyl)(methyl) amino)-5-methyl-6-oxo-5,6-dihydro-1,5- naphthyridine-2,7-dicarbonitrile |
| 213 | 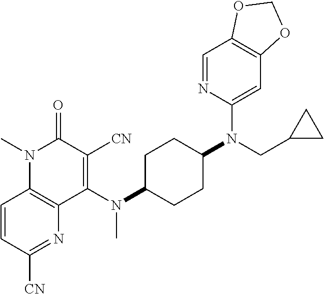 | cis-8-((-4-([1,3]dioxolo[4,5-c]pyridin-6- yl(cyclopropylmethyl)amino)cyclohexyl)(methyl) amino)-5-methyl-6-oxo-5,6-dihydro-1,5- naphthyridine-2,7-dicarbonitrile |
| 214 | 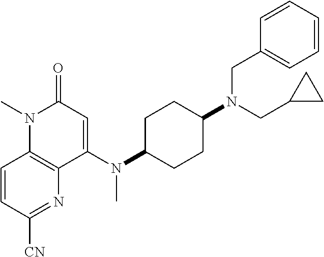 | cis-8-((-4-(benzyl(cyclopropylmethyl)amino) cyclohexyl)(methyl)amino)-5-methyl-6-oxo-5,6- dihydro-1,5-naphthyridine-2-carbonitrile |

### Pharmaceutical Compositions

In certain embodiments, are provided pharmaceutical compositions comprising a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof.

In some embodiments, the pharmaceutical compositions further comprise a pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In other embodiments, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog or salt thereof (e.g., a carrier capable of suspending or dissolving the active compound) and having the properties of being nontoxic and non-inflammatory in a patient.

Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

Inhibiting DGK Activity and Treating Diseases Associated with DGKα and/or DGKζ

In certain embodiments, described herein, are methods for inhibiting the activity of at least one diacylglycerol kinase comprising contacting the diacylglycerol kinase with a compound as described herein, or a pharmaceutically acceptable salt, solvate, hydrate, isomer, isotope, or composition thereof. In some embodiments, the diacylglycerol kinase is diacylglycerol kinase alpha (DGKα) or diacylglycerol kinase zeta (DGKζ). In certain embodiments, methods of treating a subject having a disease or disorder associated with the activity of DGKα, DGKζ, or both DGKα and DGKζ are disclosed, the method comprising administering to a subject in need thereof a pharmaceutically effective amount of a compound of structure (I), or a pharmaceutically acceptable salt, solvate, hydrate, isomer, isotope, or composition thereof.

As used herein, the term “administering” or “administration” refers to providing a compound, a pharmaceutical composition comprising the same, to a subject by any acceptable means or route, including (for example) by oral, parenteral (e.g., intravenous), or topical administration.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed. The terms cover the treatment of a disease-state in a mammal, particularly in a human, and include:
(a) preventing the disease-state from occurring in a mammal, in particular, when such mammal is predisposed to the disease state but has not yet been diagnosed as having it;
(b) inhibiting the disease-state, i.e., arresting its development: and/or
(c) relieving the disease-state, i.e., causing regression of the disease state.

As used herein, the term “DGK-mediated” or “DGK-modulated” or “DGK-dependent” diseases or disorders means any disease or other deleterious condition in which DGK, or a mutant thereof, is known to play a role. Accordingly, another embodiment of the present application relates to treating or lessening the severity of one or more diseases in which DGKα, DGKζ, or both DGKα and DGKζ, or a mutant thereof, are known to play a role. Specifically, the present application relates to a method of treating or lessening the severity of a disease or condition selected from a viral infection or a proliferative disorder, such as cancer, wherein said method comprises administering to a patient in need thereof a compound of structure (I), or a pharmaceutically acceptable salt, solvate, hydrate, isomer, isotope, or composition thereof, according to the present application.

As used herein, the term “subject” refers to an animal (e.g., a mammal, such as a human). A subject to be treated according to the methods described herein may be one who has been diagnosed with a viral infection or proliferative disorder, such as cancer. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the term “therapeutically effective amount” or “pharmaceutically effective amount” is intended to include an amount of a compound of the present invention alone or an amount of a compound of the present invention in combination with other active ingredients effective to act as an inhibitor of DGKα and/or DGKζ or effective to treat or prevent viral infections and proliferative disorders, such as cancer.

As used herein, the terms “modulate”, or “modulating” refer to the ability to increase or decrease the activity of one or more kinases. Accordingly, compounds of the invention can be used in methods of modulating a kinase by contacting the kinase with any one or more of the compounds or compositions described herein. In some embodiments, the compounds can act as inhibitors of one or more kinases. In some embodiments, the compounds can act to stimulate the activity of one or more kinases. In further embodiments, the compounds of the invention can be used to modulate activity of a kinase in an individual in need of modulation of the receptor by administering a modulating amount of a compound as described herein.

As used herein, the term “contacting” refers to the bringing together of indicated moieties in an in vitro system or an in vivo system. For example, “contacting” the DGKα and DGKζ enzyme with a compound of Structure (I) includes the administration of a compound of the present invention to an individual or patient, such as a human, having DGKα and DGKζ, as well as, for example, introducing a compound of structure (I) into a sample containing a cellular or purified preparation containing DGKα and DGKζ enzyme.

The term “DGKα and DGKζ inhibitor” refers to an agent capable of inhibiting the activity of diacylglycerol kinase alpha and/or diacylglycerol kinase zeta (DGKα and DGKζ) in T cells resulting in T cell stimulation. The DGKα and DGKζ inhibitor may be a reversible or irreversible DGKα and DGKζ inhibitor. A “reversible DGKα and DGKζ inhibitor” is a compound that reversibly inhibits DGKα and DGKζ enzyme activity either at the catalytic site or at a non-catalytic site and “an irreversible DGKα and DGKζ inhibitor” is a compound that irreversibly destroys DGKα and DGKζ enzyme activity by forming a covalent bond with the enzyme.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The compounds of structure (I) can inhibit activity of diacylglycerol kinase alpha (DGKα) and/or diacylglycerol kinase zeta (DGKαζ). For example, the compounds of structure (I) can be used to inhibit activity of DGKα and DGKζ in a cell or in an individual in need of modulation of DGKα and DGKζ by administering an inhibiting amount of a compound of structure (I) or a salt thereof.

The compounds of structure (I) and pharmaceutical compositions comprising at least one compound of structure (I) are useful in treating or preventing any disease or condition associated with DGK target inhibition in T cells. These include viral and other infections (e.g., skin infections, GI infection, urinary tract infections, genito-urinary infections, systemic infections), and proliferative diseases (e.g., cancer).

In some embodiments, are provided methods of inhibiting a kinase comprising contacting the kinase with an effective amount of a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof, or composition thereof, for the treatment of proliferative disorders or viral infections.

In some embodiments the kinase is DGK. In some embodiments the kinase is DGKα. In some embodiments the kinase is DGKζ.

In some embodiments, are methods for treating a DGK dependent condition, comprising administering to a subject in need thereof, an effective amount of a compound of structure (I), or a pharmaceutically acceptable salt, solvate, hydrate, isomer, isotope, or pharmaceutical composition thereof. In some embodiments, the DGK dependent condition is a DGKα dependent condition. In some embodiments, the DGK dependent condition is a DGKζ dependent condition. In some embodiments the DGK dependent condition is an infection. In some embodiments the DGK dependent condition is a viral infection. In some embodiments the DGK dependent condition is cancer.

In some embodiments are uses of a compound of structure (I), or a pharmaceutically acceptable salt, solvate, hydrate, isomer, tautomer, isotope, or pharmaceutical composition thereof in the manufacture of a medicament.

In some embodiments the medicament is for the treatment of cancer. In some embodiments the medicament is for the treatment of an autoimmune disease.

In some aspects, the invention provides a method of treating a patient suffering from or susceptible to a medical condition that is associated with DGK target inhibition in T cells. A number of medical conditions can be treated. The method comprises administering to the patient a therapeutically effective amount of a composition comprising a compound of structure (1) and/or a pharmaceutically acceptable salt thereof, a stereoisomer thereof or a tautomer thereof. For example, the compounds described herein may be used to treat or prevent viral infections and proliferative diseases such as cancer.

The present invention further provides methods of treating diseases associated with activity or expression, including abnormal activity and/or overexpression, of DGKα and DGKζ in an individual (e.g., patient) by administering to the individual in need of such treatment a therapeutically effective amount or dose of a compound of Structure (I) or a pharmaceutical composition thereof. Example diseases can include any disease, disorder or condition that is directly or indirectly linked to expression or activity of DGKα and DGKζ enzyme, such as over expression or abnormal activity. A DGKα and/or DGKζ associated disease can also include any disease, disorder or condition that can be prevented, ameliorated, or cured by modulating DGKα and DGKζ enzyme activity. Examples of DGKα and DGKζ associated diseases include cancer and viral infections such as HIV infection, hepatitis B, and hepatitis C.

The compounds of structure (I) and pharmaceutical compositions comprising at least one compound of structure (I) may be administered to animals, preferably mammals (e.g., domesticated animals, cats, dogs, mice, rats), and more preferably humans. Any method of administration may be used to deliver the compound or pharmaceutical composition to the patient. In certain embodiments, the compound of structure (I) or pharmaceutical composition comprising at least one compound of structure (I) is administered orally. In other embodiments, the compound of structure (I) or pharmaceutical composition comprising at least one compound of structure (I) is administered parenterally.

Described herein are methods of treating a subject having a proliferative disorder or a viral infection comprising administering to the subject in need thereof a pharmaceutically effective amount of a compound as described herein, or a pharmaceutically acceptable salt, solvate, hydrate, isomer, tautomer, isotope, or composition thereof.

In one embodiment, methods are provided for treating a proliferative disorder or a viral infection, comprising administering to a subject in need thereof an effective amount of the pharmaceutical composition, comprising a carrier or excipient and a compound having structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof.

Also described herein, are uses of a compound of structure (I), or a pharmaceutically acceptable salt, solvate, hydrate, isomer, isotope, or composition thereof, for inhibiting the activity of at least one of diacylglycerol kinase selected from diacylglycerol kinase alpha (DGKα) and diacylglycerol kinase zeta (DGKζ).

In one embodiment are provided uses of a compound of structure (I), or a pharmaceutically acceptable salt, solvate, hydrate, isomer, isotope, or composition thereof, for inhibiting the activity of at least one of diacylglycerol kinase selected from diacylglycerol kinase alpha (DGKα) and diacylglycerol kinase zeta (DGKζ).

Also described herein, are uses of a compound of structure (I), or a pharmaceutically acceptable salt, solvate, hydrate, isomer, isotope, or composition thereof, for treating a disease or disorder associated with the activity of DGKα or DGKζ, or both DGKα and DGKζ.

In one embodiment, are provided uses of a compound of structure (I), or a pharmaceutically acceptable salt, solvate, hydrate, isomer, isotope, or composition thereof, for treating a disease or disorder associated with the activity of DGKα or DGKζ, or both DGKα and DGKζ.

Also provided are uses of a compound of structure (I), or a pharmaceutically acceptable salt, solvate, hydrate, isomer, isotope, or composition thereof, for the treatment of proliferative disorders or viral infections.

In one embodiment are provided uses of a compound of structure (I), or a pharmaceutically acceptable salt, solvate, hydrate, isomer, isotope, or composition thereof, for the treatment of proliferative disorders or viral infections.

### Cancer

In some embodiments, the proliferative disorder is cancer. Thus, in some aspects, the invention provides methods of treating cancer associated with activity or expression, including abnormal activity and/or overexpression, of DGKα and DGKζ in an individual (e.g., patient) by administering to the individual in need of such treatment a therapeutically effective amount or dose of a compound of structure (I) or a pharmaceutical composition thereof.

Types of cancers that may be treated with the compound of structure (I) include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

In some embodiments, the cancer is cancer of the colon, pancreatic cancer, breast cancer, prostate cancer, lung cancer, ovarian cancer, cervical cancer, renal cancer, bladder cancer, cancer of the head and neck, lymphoma, leukemia, or melanoma. In some embodiments, the cancer is colon cancer. In some embodiments, the cancer is pancreatic cancer. In some embodiments, the cancer is breast cancer. In some embodiments, the cancer is prostate cancer. In some embodiments, the cancer lung cancer. In some embodiments, the cancer is ovarian cancer. In some embodiments, the cancer is cervical cancer. In some embodiments, the cancer is renal cancer. In some embodiments, the cancer is renal cancer. In some embodiments, the cancer is cancer of the head and neck. In some embodiments, the cancer is lymphoma. In some embodiments, the cancer is leukemia. In some embodiments, the cancer is melanoma.

In one embodiment, are provided uses of a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof, or composition thereof, for the treatment of proliferative disorders.

In some embodiments, the proliferative disorder is cancer. In some embodiments, the cancer is cancer of the colon, pancreatic cancer, breast cancer, prostate cancer, lung cancer, ovarian cancer, cervical cancer, renal cancer, cancer of the head and neck, lymphoma, leukemia and melanoma.

Also provided are uses of a compound of structure (I), or a pharmaceutically acceptable salt, solvate, hydrate, isomer, isotope, or composition thereof, for the manufacture of a medicament.

### Infections

In other aspects, the invention provides methods of treating infections associated with activity or expression, including abnormal activity and/or overexpression, of DGKα and DGKζ in an individual (e.g., patient) by administering to the individual in need of such treatment a therapeutically effective amount or dose of a compound of structure (I) or a pharmaceutical composition thereof. In some embodiments, the infections are viral infections. In some embodiments, the infections are chronic viral infections.

In one embodiment, are provided uses of a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof, or composition thereof, for the treatment of viral infections.

Chronic viral infections that may be treated using the present combinatorial treatment include, but are not limited to, diseases caused by: hepatitis C virus (HCV), human papilloma virus (HPV), cytomegalovirus (CIVIV), herpes simplex virus (HSV), Epstein-Barr virus (EBV), varicella zoster virus, coxsackie virus, human immunodeficiency virus (HIV) Notably, parasitic infections (e.g., malaria) may also be treated by the above methods wherein compounds known to treat the parasitic conditions are optionally added in place of the antiviral agents.

### Combination Therapy

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g. IL2 and GM-CSF), and/or tyrosine kinase inhibitors can be optionally used in combination with the compounds of structure (I) for treatment of DGKα and DGKζ associated diseases, disorders or conditions. The agents can be combined with the present compounds in a single dosage form, or the agents can be administered simultaneously or sequentially as separate dosage forms.

In some embodiments, the pharmaceutical composition comprising a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof, with at least one pharmaceutically acceptable carrier, diluent, or excipient further comprises a second therapeutic agent.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral mutes, intravenous mutes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally, or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-dmg treatment may be conducted at any suitable time so long as a beneficial effect from the co-action of the combination of the therapeutic agents and non-dmg treatment is achieved. For example, in appropriate cases, the beneficial effect is still achieved when the non-drug treatment is temporally removed from the administration of the therapeutic agents, perhaps by days or even weeks.

In some aspects, the present invention provides a combined preparation of a compound of structure (I), and/or a pharmaceutically acceptable salt thereof, a stereoisomer thereof or a tautomer thereof: and additional therapeutic agent(s) for simultaneous, separate or sequential use in the treatment and/or prophylaxis of multiple diseases or disorders associated with DGK target inhibition in T cells.

In one aspect, T cell responses can be stimulated by a combination of a compound of Structure (I) and one or more of:
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; and
(ii) an agonist of a protein that stimulates T cell activation such as B7-1, B7-2, CD28, 4-1BB (CD137), 4-1BBL, ICOS, ICOS-L, OX40, OX40L, GITR. GITRL, CD70, CD27, CD40, DR3 and CD28H.
   Combination with Anti-Cancer Agents

In another aspect, compounds of structure (I) may be administered in combination with an anti-cancer agent. Anti-cancer agents include, for example, small molecule drugs, antibodies, or other biologic or small molecule. Examples of biologic immuno-oncology agents include, but are not limited to, cancer vaccines, antibodies and cytokines. In one aspect, the antibody is a monoclonal antibody. In another aspect, the monoclonal antibody is humanized or human.

In one aspect the immuno-oncology agent is an agonist of a stimulatory (including a co-stimulatory) receptor; or an antagonist of an inhibitory (including a co-inhibitory) signal on T cells, both of which result in amplifying antigen-specific T cell responses (often referred to as immune checkpoint regulators). Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-HI (PD-L1), B7-DC (PD-L2), B7-H2 (ICOS-L), B7-H3, B7-H4, B7-H5 (VISTA), and B7-H6. Another family of membrane bound ligands that bind to costimulatory or coinhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEG1/TL1A, TRAMP/DR3, EDAR, EDA1, XEDAR, EDA2, TNFR1, Lymphotoxin α/TNFβ, TNFR2, TNFα, LTβR, Lymphotoxin a 102, FAS, FASL, RELT, DR6, TROY, NGFR.

Yet other agents for combination therapies for the treatment of cancer include antagonists of inhibitory receptors on NK cells or agonists of activating receptors on NK cells. For example, antagonists of KIR, such as lirilumab.

Yet other agents for combination therapies for the treatment of cancer include agents that inhibit or deplete macrophages or monocytes, including but not limited to CSF-1R antagonists such as CSF-1R antagonist antibodies including RG-7155 or FPA-008.

Yet other agents for combination therapies for the treatment of cancer include agonistic agents that ligate positive co-stimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment, e.g., block inhibitory receptor engagement, such as PD-L1/PD-1 interactions; deplete or inhibit Tregs, such as using an anti-CD25 monoclonal antibody (e.g., daclizumab); or by ex vivo anti-CD25 bead depletion; inhibit metabolic enzymes such as IDO, or reverse/prevent T cell anergy or exhaustion; and agents that trigger innate immune activation and/or inflammation at tumor sites.

Yet other agents for combination therapies for the treatment of cancer include CTLA-4 antagonists such as an antagonistic CTLA-4 antibody. Suitable CTLA-4 antibodies include, for example, YERVOY (ipilimumab) or tremelimumab.

Yet other agents for combination therapies for the treatment of cancer include PD-1 antagonists, such as an antagonistic PD-1 antibody. Suitable PD-1 antibodies include, for example, OPDIVO (nivolumab), KEYTRUDA (pembrolizumab), MEDI-0680 (AMP-514; WO2012/145493) or pidilizumab (CT-011). Another approach to target the PD-1 receptor is the recombinant protein composed of the extracellular domain of PD-L2 (B7-DC) fused to the Fe portion of IgG1, called AMP-224.

Yet other agents for combination therapies for the treatment of cancer include PD-L1 antagonists, such as an antagonistic PD-L1 antibody Suitable PD-L1 antibodies include, for example, MPDL3280A (RG7446; WO2010/077634), durvaluma (MEDI4736), BMS-936559 (WO2007/005874), and MSB0010718C (WO2013/79174).

Yet other agents for combination therapies for the treatment of cancer include LAG-3 antagonists, such as an antagonistic LAG-3 antibody. Suitable LAG3 antibodies include, for example, BMS-986016 (WO10/19570, WO14/08218), or IMP-731 or IMP-321 (WO08/132601, WO09/44273).

Yet other agents for combination therapies for the treatment of cancer include CD137 (4-1BB) agonists, such as an agonistic CD137 antibody. Suitable CD137 antibodies include, for example, urelumab and PF-05082566 (WO12/32433).

Yet other agents for combination therapies for the treatment of cancer include GITR agonists such as an agonistic GITR antibody. Suitable GITR antibodies include, for example, BMS-986153, BMS-986156, TRX-518 (WO06/105021, WO09/009116) and MK-4166 (WO11/028683).

Yet other agents for combination therapies for the treatment of cancer include IDO antagonists. Suitable IDO antagonists include, for example, INCB-024360 (WO2006/122150, WO07/75598, WO08/36653, WO08/36642), indoximod, BMS-986205, or NLG-919 (WO09/73620, WO09/1156652, WO11/56652, WO12/142237).

Yet other agents for combination therapies for the treatment of cancer include OX40 agonists, such as an agonistic OX40 antibody. Suitable OX40 antibodies include, for example, MEDI-6383 or MEDI-6469.

Yet other agents for combination therapies for the treatment of cancer include OX40L antagonists, such as an antagonistic OX40L antibody Suitable OX40L antagonists include, for example, RG-7888 (WO06/029879).

Yet other agents for combination therapies for the treatment of cancer include CD40 agonists, such as an agonistic CD40 antibody.

Yet other agents for combination therapies for the treatment of cancer include CD40 antagonists, such as an antagonistic CD40 antibody. Suitable CD40 antibodies include, for example, lucatumumab or dacetuzumab.

Yet other agents for combination therapies for the treatment of cancer include CD27 agonists, such as an agonistic CD27 antibody. Suitable CD27 antibodies include, for example, varlilumab.

Yet other agents for combination therapies for the treatment of cancer include, for example, alkylating agents (including, without limitation, nitrogen mustards, ethylenimine derivatives, alkyl sulfonates, nitrosoureas and triazenes) such as uracil mustard, 5 chlormethine, cyclophosphamide (CYTOXAN), ifosfamide, melphalan, chlorambucil pipobroman, triethylene-melamine, triethylenethiophosphoramine, busulfan, carmustine, lomustine, streptozocin, dacarbazine, and temozolomide. Suitable chemotherapeutic or other anti-cancer agents further include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine. Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formcin, mitomycin-C, L-asparaginase, interferons (especially IFN-α), etoposide, and teniposide. Suitable chemotherapeutic or other anti-cancer agents further include, for example, epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; haematopoietic growth factors; navelbene, CPT-11, anastrazole, letrazole, capecitabine, reloxafine, droloxafine; antibody therapeutics such as trastuzumab (HERCEPTIN), antibodies to costimulatory molecules such as CTLA-4, 4-1BB and PD-1, or antibodies to cytokines (IL-1O or TGF-β); and agents that block immune cell migration such as antagonists to chemokine receptors, including CCR2 and CCR4.

Yet other agents for combination therapies for the treatment of cancer include anti-cancer vaccines, including dendritic cells, synthetic peptides, DNA vaccines and recombinant viruses.

Yet other agents for combination therapies for the treatment of cancer include signal transduction inhibitors (STI). A “signal transduction inhibitor” is an agent that selectively inhibits one or more vital steps in signaling pathways, in the normal function of cancer cells, thereby leading to apoptosis. Suitable STI's include, but are not limited to:
(i) bcr/abl kinase inhibitors such as, for example, STI 571 (GLEEVEC);
(ii) epidermal growth factor (EGF) receptor inhibitors such as, for example, kinase inhibitors (IRESSA, SSI-774) and antibodies (Imclone: C225 [Goldstein et al, Clin. Cancer Res, 1995, 1, 1311-1318; and Abgenix: ABX-EGF);
(iii) her-2/neu receptor inhibitors such as farnesyl transferase inhibitors (FTI) such as, for example, L-744,832 (Kohl et al, Nat. Med., 1995, 1(8), 792-797);
(iv) inhibitors of Akt family kinases or the Akt pathway, such as, for example, rapamycin;
(v) cell cycle kinase inhibitors such as, for example, flavopiridol and UCN-01; and
(vi) phosphatidyl inositol kinase inhibitors such as, for example, LY294002.

In the treatment of melanoma, suitable agents for use in combination with the compounds of structure (I) include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOYTM. Compounds of Structure (I) may also be combined with immunotherapy drugs, including cytokines such as interferon alpha, interleukin 2, and tumor necrosis factor (TNF) in the treatment of melanoma.

Compounds of structure (I) may also be used in combination with vaccine therapy in the treatment of melanoma. Anti-melanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas confined to the arms or legs may also be treated with a combination of agents including one or more compounds of structure (I), using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects.

Combination with Anti-Viral Agents

Suitable antiviral agents contemplated for use in combination with the compound of structure (I) include nucleoside and nucleotide reverse transcriptase inhibitors (NRTIs), non-nucleoside reverse transcriptase inhibitors (NNRTis), protease inhibitors and other antiviral drugs.

Examples of suitable NRTIs include zidovudine (AZT); didanosine (ddl); zalcitabine (ddC); stavudine (d4T); lamivaidine (3TC): abacavir (1592U89); adefovir dipivoxil [bis(POM)-PMEA]; lobucavir (BMS-180194); BCH-I0652, emitricitabine [(−)-FTC]; beta-L-FD4 (also called beta-L-D4C and nanled beta-L-2′,3′-dicleoxy-5-fluorocytidene); DAPD, ((−)-beta-D-2,6-diamino-purine dioxolane); and lodenosine (FddA).

Examples of suitable NNRTIs include nevirapine (BI-RG-587); delaviradine (BHAP, U-90152); efavirenz (DMP-266); PNU-142721, AG-1549; MKC-442 (1-(ethoxy-methyl)-5-(]-methylethyl)-6-(phenylmethyl)-(2,4(1H,3H)-pyrimidinedione); and (+)-calanolide A (NSC-675451) and B.

Examples of suitable protease inhibitors include saquinavir (Ro 31-8959); ritonavir (ABT-538); indinavir (MK-639); nelfinavir (AG-1343): amprenavir (141W94); lasinavir (BMS-234475): DMP-450; BMS-2322623, ABT-378; and AG-1549.

Other antiviral agents include hydroxyurea, ribavirin, IL-2, IL-12, pentafuside and Yissum Project No. 11607.

The present invention further provides pharmaceutical compositions comprising at least one compound of structure (I), a pharmaceutically acceptable carrier, optionally, at least one chemotherapeutic drug, and, optionally, at least one antiviral agent.

### Routes of Administration

The compounds of this invention can be administered for any of the uses described herein by any suitable means, for example, orally, such as tablets, capsules (each of which includes sustained release or timed release formulations), pills, powders, granules, elixirs, tinctures, suspensions (including nanosuspensions, micro suspensions, spray-dried dispersions), syrups, and emulsions; sublingually; buccally; parenterally, such as by subcutaneous, intravenous, intramuscular, or intratarsal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

In one embodiment, the invention provides an oral pharmaceutical composition comprising a compound of structure (I) or any of the structures (I-A), (I-B), (I-B-cis), (I-B-trans), (I-A-1), (I-A-2), (I-A-3), (I-A-4), (I-B-1), (I-B-2), (I-B-3) or (I-B-4) or a pharmaceutically acceptable salt, solvate, hydrate, isomer or isotope thereof, together with at least one pharmaceutically acceptable oral carrier, diluent, or excipient.

### Kits

The present invention also includes pharmaceutical kits useful, for example, in the treatment or prevention of DGKα and DGKζ associated diseases or disorders, and other diseases referred to herein, which include one or more containers containing a pharmaceutical composition comprising a therapeutically effective amount of a compound of structure (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

### General Methods

Compounds having structure (I) can be synthesized using standard synthetic techniques known to those of skill in the art. For example, compounds of the present disclosure can be synthesized using the general synthetic procedures described below and set forth in Schemes 1-14.

Modifications to these methods will be apparent to one skilled in the art. To this end, the reactions, processes and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed. Compounds are prepared using standard organic chemistry techniques such as those described in, for example, March's Advanced Organic Chemistry, 7th Edition, John Wiley and Sons, Inc. (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4th Edition, Wiley-Interscience. (2006). All starting materials and reagents are commercially available or readily prepared.

### Compounds of Structure (I-A-1)

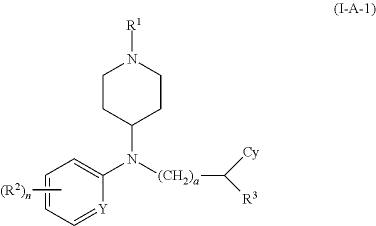

Coupling of 1-protected piperidin-4-one with the appropriate aniline, provides a 1-protected-N-arylpiperidin-4-amine intermediate, as shown in scheme 1.

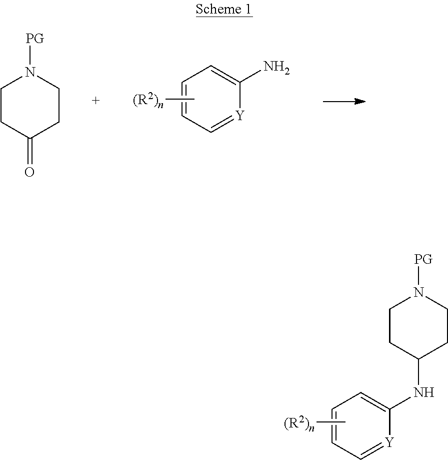

The cycloalkyl or heterocycloalkyl ring (Cy) is introduced by reaction with cycloalkyl or heterocycloalkyl methyl bromide, cycloalkyl or heterocycloalkyl —R3 ketone or cycloalkyl or heterocycloalkyl-aldehyde. Removal of the piperidine protecting group and coupling with R¹ chloride provides the final desired compound, as shown in scheme 2.

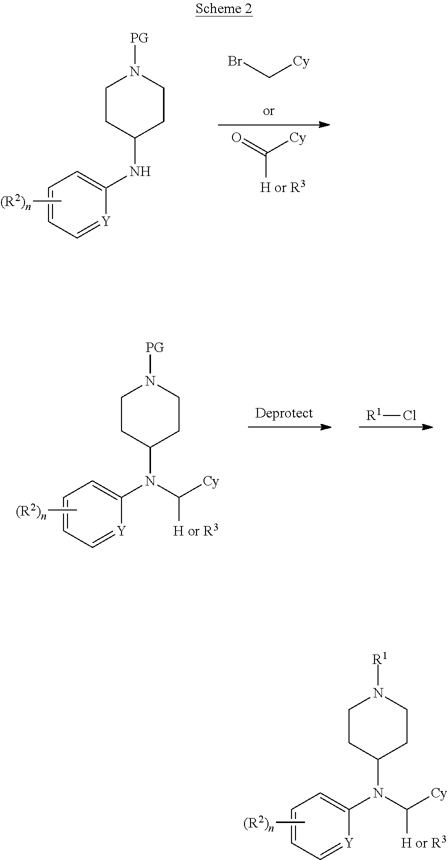

### Compounds of Structure (I-A-2)

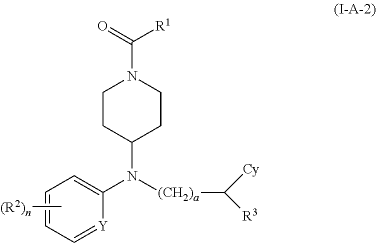

The aniline intermediate may be prepared as described above and shown in schemes 1 and 2. The R¹—C(O)— group is introduced by reaction with R¹—COOH or R¹—COCl to provide the final desired compound, as shown in scheme 3.

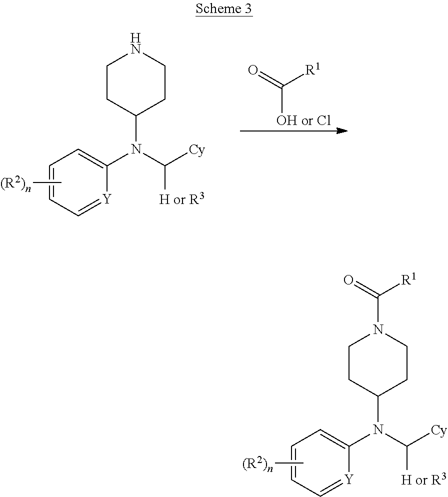

### Compounds of Structure (I-A-3) or Structure (I-A-4),

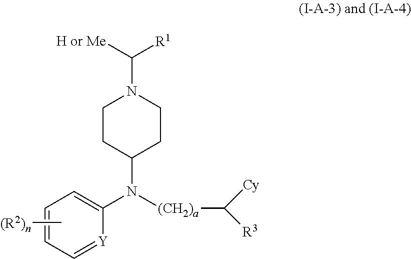

The aniline intermediate may be prepared as described above and shown in schemes 1 and 2. The —CH₂-R¹ or —CHMe-R¹ group is introduced by reaction with R¹ ketone or R¹-aldehyde to provide the final desired compound, as shown in scheme 4.

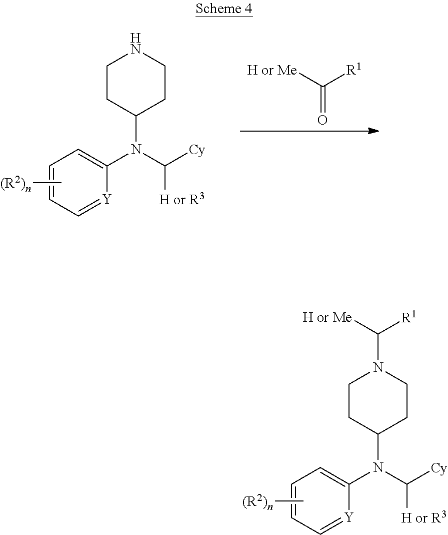

### Compounds of Structure (I-B-1)

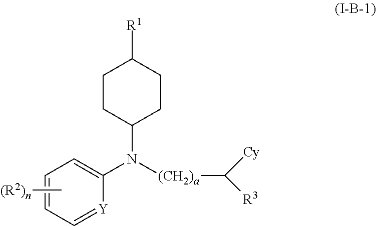

The mono protected, mono-substituted diamino cyclohexane intermediate may be prepared by coupling an aryl bromide with monoprotected diamino cyclohexane (scheme 5), or alternatively, coupling an aniline with an amino protected 4-aminocyclohexan-1-one (scheme 6), as shown below:

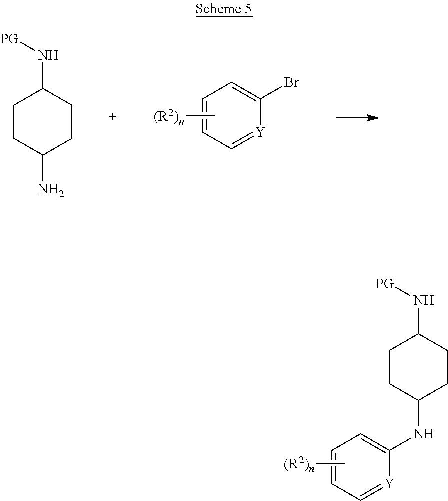

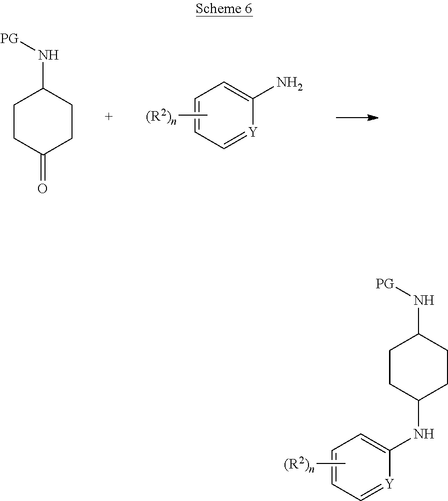

The cycloalkyl or heterocycloalkyl ring is introduced by reaction with ethoxy-Cy-OTMS (a is 0) according to scheme 7 or cyclo-carbaldehyde (a is 1 or 2), according to scheme 8, and the amine is then deprotected.

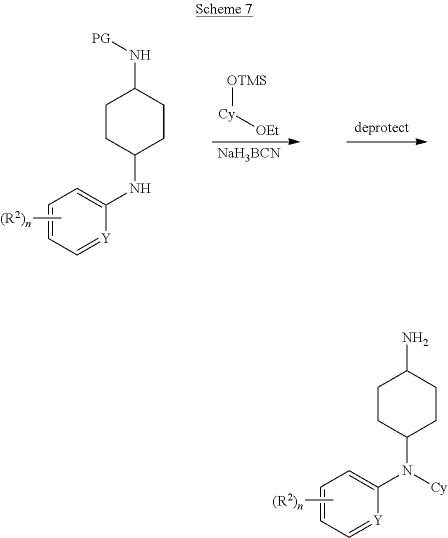

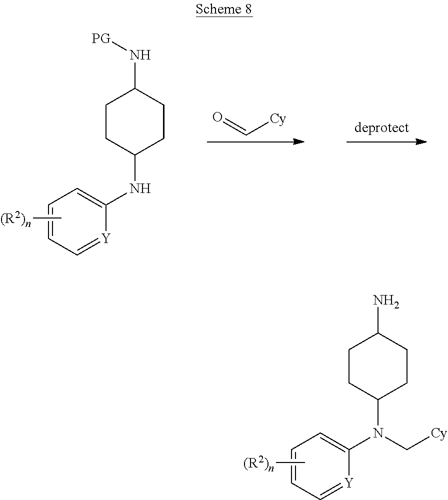

The R¹ heteroaryl ring is then synthesized according to standard heterocyclic chemistry, well known to those of skill in the art. As an illustrative example, see scheme 9 below, to prepare R¹ as a substituted pyridopyrazinone.

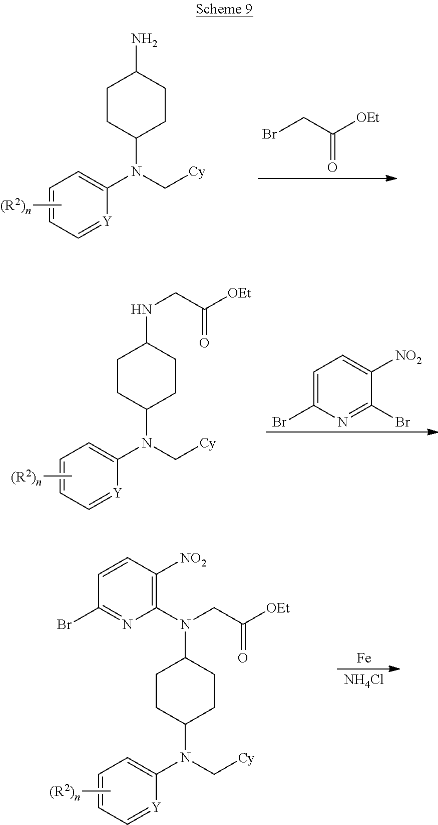

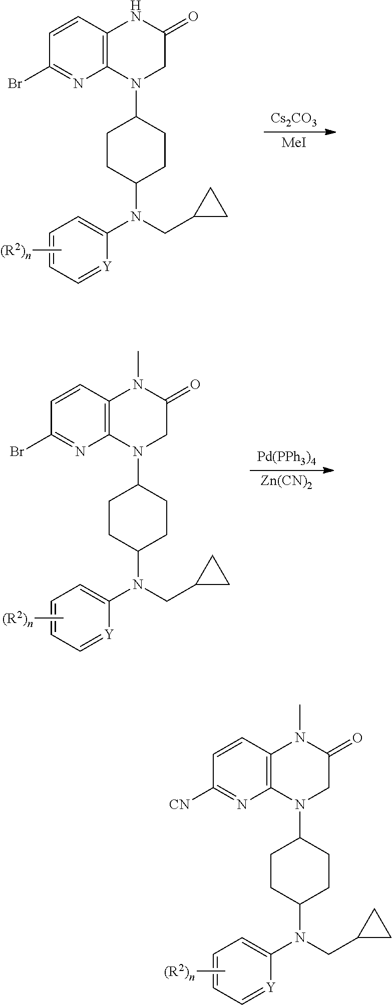

### Compounds of Structure (I-B-2) or (I-B-3)

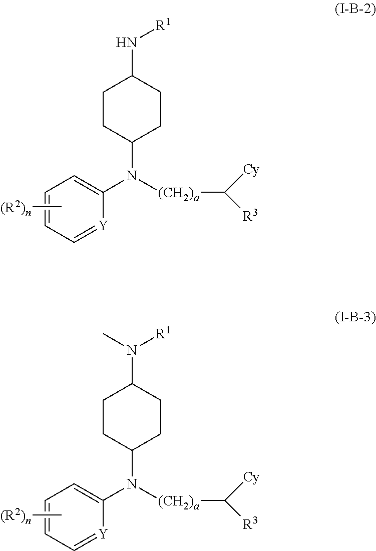

The mono protected, mono-substituted diamino cyclohexane intermediate may be prepared in the same manner as for compounds of structure (I-B-1), as described above and in schemes 5 and 6. The cycloalkyl or heterocycloalkyl ring may be introduced in the same manner as for Compounds of structure (I-B-1), as described above and shown in schemes 7 and 8.

The aniline amine is deprotected and coupled with R¹ chloride to produce the final desired product (L is NH). For compounds of structure (I-B-3), wherein L is NMe, reaction with methyl iodide introduces the methyl group, before or after coupling with R¹ chloride, depending on the specific compounds and protecting groups employed, as shown in schemes 10 and 11 below.

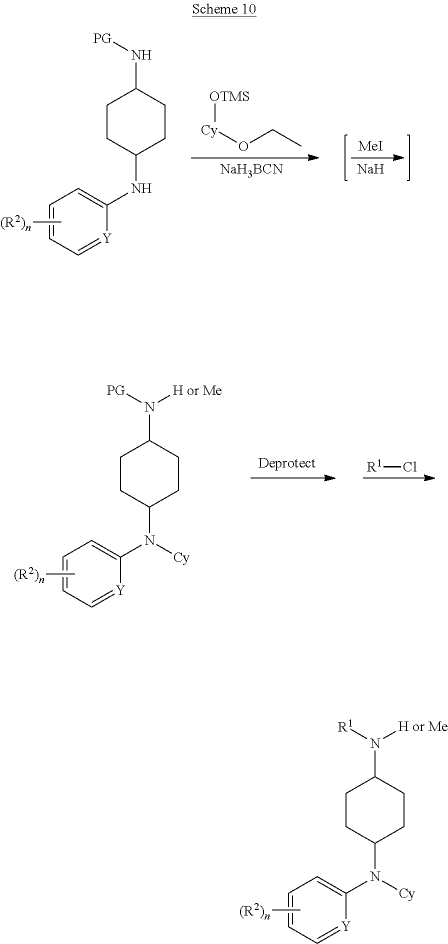

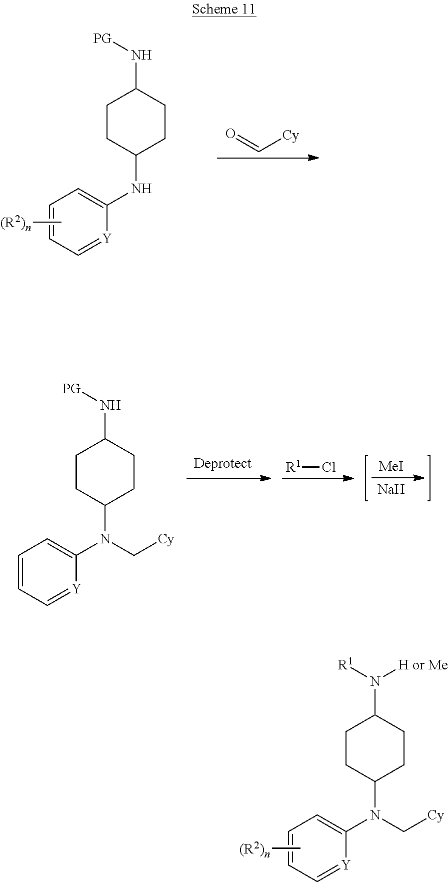

In a similar manner, compounds of structure IB2 or IB3, wherein R^{2a} is hydroxy may be prepared according to scheme 12 as shown below, employing hydroxy protecting groups as appropriate.

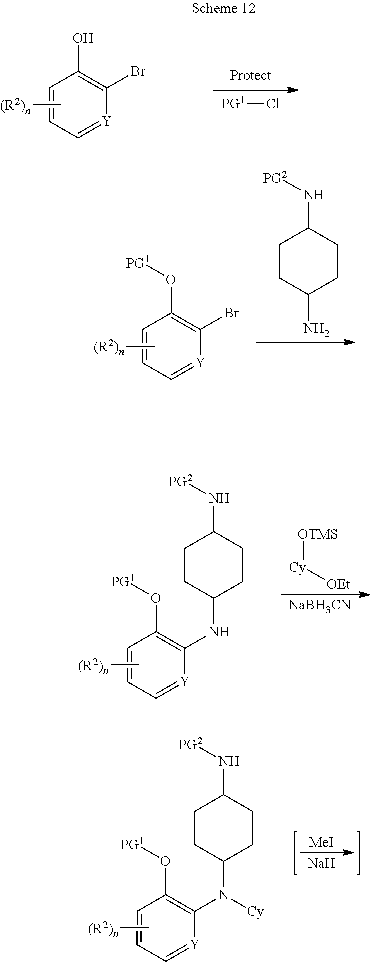

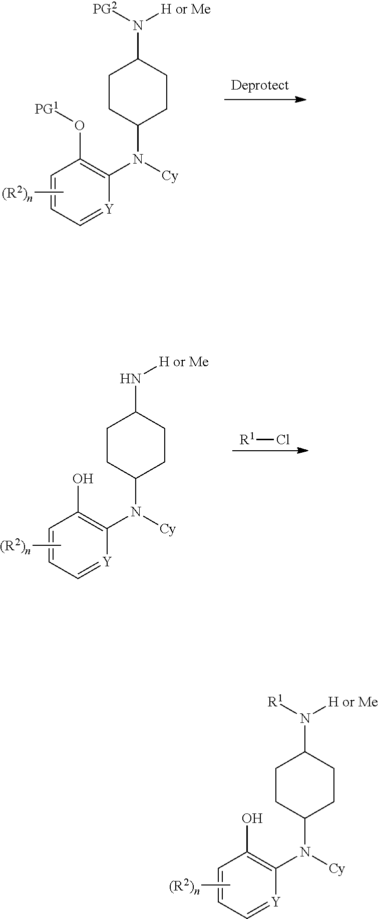

### Compounds of Structure (I-B-4)

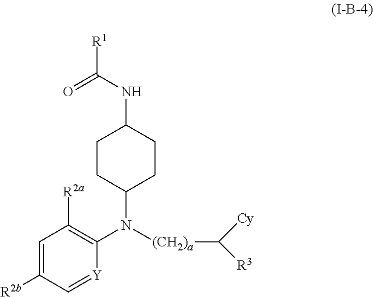

The 1-protected-N-arylpiperidin-4-amine intermediate, prepared as described above, is then coupled with cyclo-carbaldehyde, deprotected and coupled with R¹ carboxylic acid, according to scheme 13. Alternatively, the 1-protected-N-arylpiperidin-4-amine intermediate is first deprotected followed by reaction with R¹ carboxylic acid to form the amide, which is then coupled with the cycloalkyl or heterocycloalkyl ring moiety, according to scheme 14.

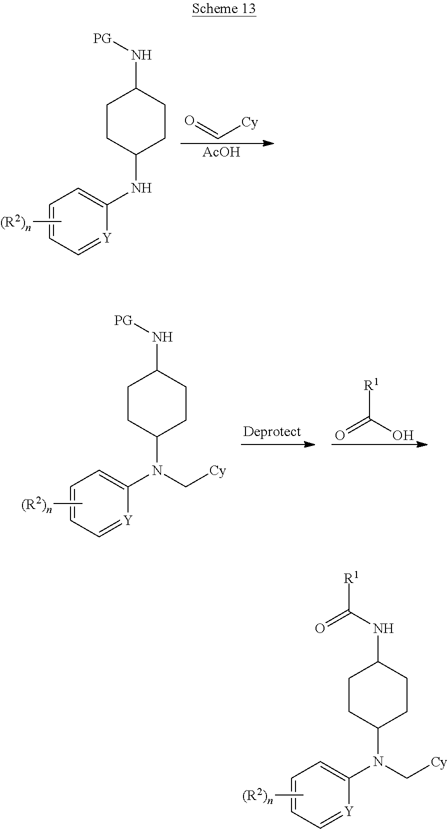

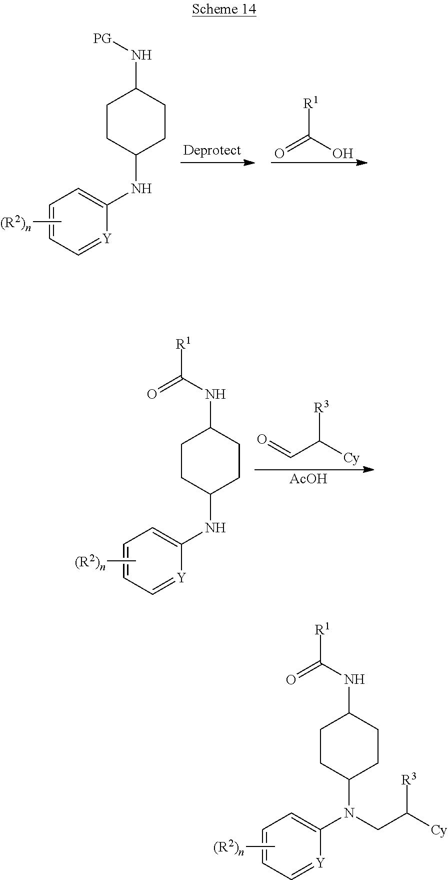

### EXAMPLES

The following examples are provided for illustrative purposes only and not to limit the scope of the claims provided herein. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Chemical Syntheses

### Example 1

### Compounds of Structure (I-A-1)

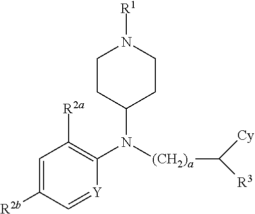

### Example 1A: Synthesis of 8-(4-((Cyclopropylmethyl)(2-hydroxyphenyl)amino)piperidin-1-yl)-5-methyl-6-oxo-5,6-dihydro-1,5-naphthyridine-2-carbonitrile

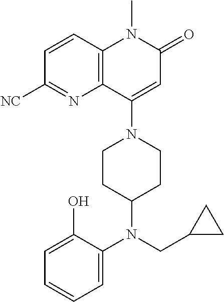 [SMILES: Cn1c(=O)cc(N2CCC(N(CC3CC3)c3ccccc3O)CC2)c2nc(C#N)ccc21]

### Step 1: tert-Butyl 4-((cyclopropylmethyl)(2-hydroxyphenyl)amino)piperidine-1-carboxylate

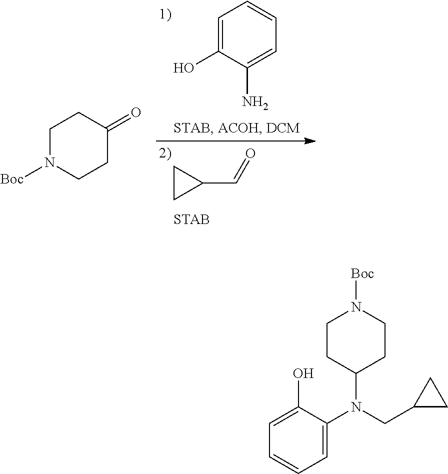 [SMILES: Nc1ccccc1O ; CC(C)(C)OC(=O)N1CCC(=O)CC1 ; O=CC1CC1 ; CC(C)(C)OC(=O)N1CCC(N(CC2CC2)c2ccccc2O)CC1]

A solution of tert-butyl 4-oxopiperidine-1-carboxylate (9.13 g, 45.8 mmol), 2-aminophenol (5.0 g, 45.8 mmol, 3.76 mL), acetic acid (50 mL) and sodium triacetoxyborohydride (19.4 g, 91.6 mmol) in DCM (100 mL) was stirred for 2 h at rt. Cyclopropane carbaldehyde (4.82 g, 68.7 mmol, 5.14 mL) and sodium triacetoxyborohydride (14.6 g, 68.7 mmol) were then added, and the resulting mixture was stirred for 1.5 h at rt. The mixture was poured into water (200 mL) and extracted with DCM (3×150 mL). The combined organic layers were washed with brine (500 mL), dried over anhydrous sodium sulfate, and concentrated under reduced pressure. The residue was purified by silica gel chromatography with petroleum ether/ethyl acetate (10:1) to afford the title compound (12.0 g, 75%) as a yellow solid.

LCMS (ES, m/z): 347.25 [M+H]⁺.

### Step 2: 2-((Cyclopropylmethyl)(piperidin-4-yl)amino)phenol hydrotrifluoroacetate

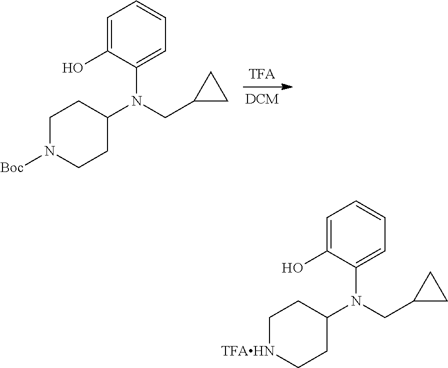

A solution of tert-butyl 4-((cyclopropylmethyl)(2-hydroxyphenyl)amino)piperidine-1-carboxylate (500 mg, 1.44 mmol) in DCM (5 mL) and TFA (2 mL) was stirred for 2 h at rt. The mixture was concentrated under reduced pressure to afford the title compound (800 mg, assumed quantitative) as a brown oil.

LCMS (ES, m/z): 247.20 [M+H]⁺.

### Step 3: 8-(4-((Cyclopropylmethyl)(2-hydroxyphenyl)amino)piperidin-1-yl)-5-methyl-6-oxo-5,6-dihydro-1,5-naphthyridine-2-carbonitrile

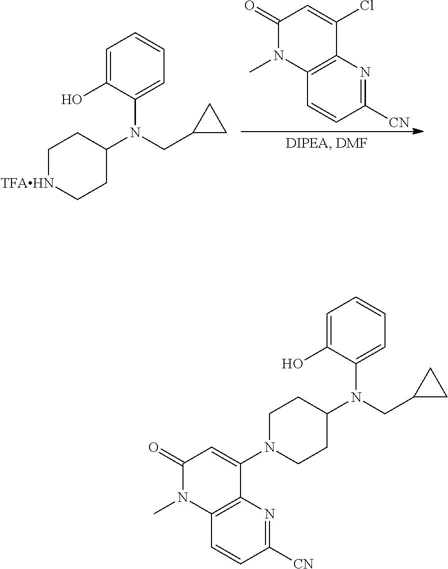

A solution of 2-((cyclopropylmethyl)(piperidin-4-yl)amino)phenol hydrotrifluoroacetate (100 mg, 405 μmol), 8-chloro-5-methyl-6-oxo-1,5-naphthyridine-2-carbonitrile (89 mg, 406 μmol) and DIPEA (1.05 g, 8.12 mmol, 1.41 mL) in DMF (3 mL) was stirred for 2 h at 140° C. The mixture was cooled to rt, diluted with water (10 mL) and extracted with ethyl acetate (3×10 mL). The combined organic layers were washed with brine (30 mL), dried over anhydrous sodium sulfate and concentrated under reduced pressure. The residue was purified by reversed phase chromatography (Column: Xselect CSH OBD, 30×150 mm, 5 um; Mobile Phase, A: Water (10 mmol/L NH₄HCO₃+0.1% NH₃·H₂O) and B: ACN (63% to 83% in 7 min); Detector: 220 nm). The collected fraction was lyophilized to afford the title compound (28.2 mg, 16%) as a light-yellow solid.

¹H-NMR (DMSO-d₆, 400 MHz) δ (ppm): 8.30 (s, 1H), 8.16 (d, J=8.8 Hz, 1H), 8.06 (d, J=8.9 Hz, 1H), 7.23-7.21 (m, 1H), 7.02-6.98 (m, 1H), 6.83-6.77 (m, 2H), 6.07 (s, 1H), 4.03-3.99 (m, 2H), 3.52 (s, 3H), 3.18-3.12 (m, 1H), 2.88-2.79 (m, 4H), 1.92-1.89 (m, 2H), 1.60-1.52 (m, 2H), 0.78-0.63 (m, 1H), 0.29-0.21 (m, 2H), 0.00-0.08 (m, 2H).

LCMS (ES, m/z): 430.20 [M+H]⁺.

### Example 1B: Synthesis of 8-(4-((Cyclopropylmethyl)(phenyl)amino)piperidin-1-yl)-5-methyl-7-nitro-6-oxo-5,6-dihydro-1,5-naphthyridine-2-carbonitrile

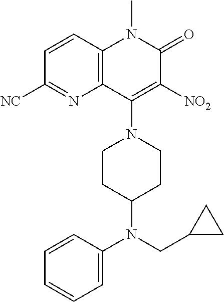 [SMILES: Cn1c(=O)c([N+](=O)[O-])c(N2CCC(N(CC3CC3)c3ccccc3)CC2)c2nc(C#N)ccc21]

### Step 1: tert-Butyl 4-(phenylamino)piperidine-1-carboxylate

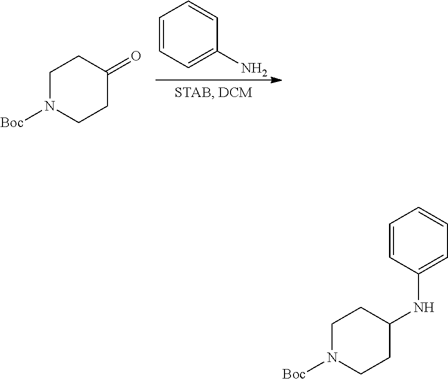

A mixture of tert-butyl 4-oxopiperidine-1-carboxylate (8.00 g, 40.2 mmol) and aniline (3.74 g, 40.2 mmol, 3.66 mL) in toluene (200 mL) was stirred for 1 h at rt. Sodium triacetoxyborohydride (10.2 g, 48.2 mmol) was added to the resulting solution and stirred for 2.5 h at rt. The reaction was quenched with water (80 mL), and the resulting solution was extracted with ethyl acetate (3×100 mL). The combined organic layers were washed with brine (100 mL), dried over anhydrous sodium sulfate, and concentrated under reduced pressure. The residue was purified by silica gel chromatography (Petroleum ether-ethyl acetate, 1:10) to afford the title compound (7.00 g, 58%) as a yellow solid.

LCMS (ES, m/z): 277.15 [M+H]⁺.

### Step 2: tert-Butyl 4-((cyclopropylmethyl)(phenyl)amino)piperidine-1-carboxylate

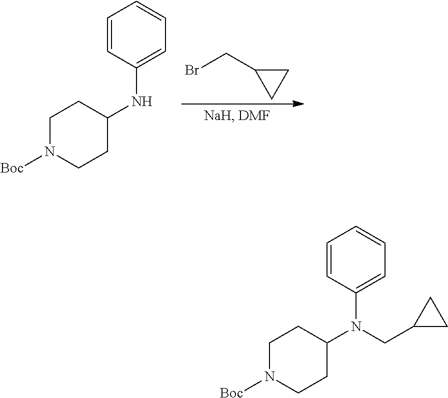

A mixture of tert-butyl 4-(phenylamino)piperidine-1-carboxylate (400 mg, 1.45 mmol) and NaH (116 mg, 2.9 mmol, 60%) in DMF (10 mL) was stirred for 0.5 h at 0° C. Bromomethylcyclopropane (195 mg, 1.45 mmol, 138 μL) was added to the resulting solution and stirred for 16 h at rt. The mixture was slowly poured into water (25 mL) and extracted with ethyl acetate (3×25 mL). The combined organic layers were washed with brine (25 mL), dried over anhydrous sodium sulfate, and concentrated under reduced pressure. The residue was purified by silica gel chromatography (Petroleum ether-ethyl acetate, 1:1) to afford the title compound (130 mg, 25%) as a colorless oil.

LCMS (ES, m/z): 331.20 [M+H]⁺.

### Step 3: N-(Cyclopropylmethyl)-N-phenyl-piperidin-4-amine hydrochloride

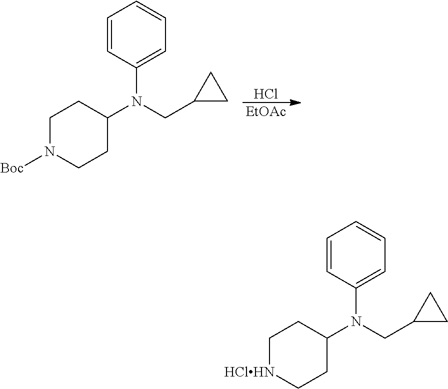

HCl (4 M in dioxane, 983 μL) was added to a solution of tert-butyl 4-((cyclopropylmethyl)-(phenyl)amino)piperidine-1-carboxylate (130 mg, 393 μmol) in ethyl acetate (2.50 mL), and the resulting mixture was stirred for 0.5 h at rt. The resulting solution was concentrated and washed with ethyl ether (5 mL). The solid was collected by filtration to afford the title compound (90 mg, 96%).

LCMS (ES, m/z): 231.20 [M+H]⁺.

### Step 4: 8-(4-((Cyclopropylmethyl)(phenyl)amino)piperidin-1-yl)-5-methyl-7-nitro-6-oxo-5,6-dihydro-1,5-naphthyridine-2-carbonitrile

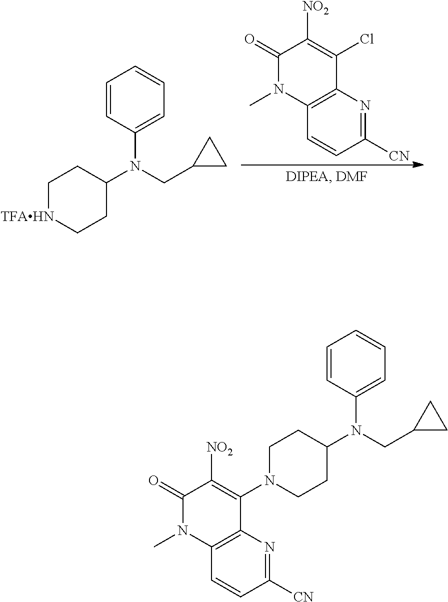

A solution of 8-chloro-5-methyl-7-nitro-6-oxo-1,5-naphthyridine-2-carbonitrile (97.7 mg, 369 μmol), N-(cyclopropylmethyl)-N-phenyl-piperidin-4-amine hydrochloride (85 mg, 369 μmol) and DIPEA (238 mg, 1.85 mmol, 321 μL) in DMF (2 mL) was stirred for 1 h at rt. The crude product was purified by reversed phase chromatography (Column: C18, 30×250 mm, 5 μm; Mobile Phase: A: water (0.05% TFA) and B: ACN (0% to 60% in 30 min); Detector: UV254 nm). The collected fractions were lyophilized to give the title compound (37.1 mg, 22%) as a yellow solid.

¹H-NMR (DMSO-d₆, 400 MHz) δ (ppm): 8.29 (d, J=8.8 Hz, 1H), 8.20 (d, J=8.8 Hz, 1H), 7.21-7.17 (m, 2H), 6.95-6.92 (m, 2H), 6.66-6.63 (m, 1H), 4.05-3.99 (m, 1H), 3.81-3.77 (m, 2H), 3.59 (s, 3H), 3.29-3.23 (m, 2H), 3.10 (d, J=5.6 Hz, 2H), 2.02-1.94 (m, 2H), 1.87-1.84 (m, 2H), 0.97-0.95 (m, 1H), 0.52-0.50 (m, 2H), 0.28-0.26 (m, 2H).

LCMS (ES, m/z): 459.25 [M+H]⁺.

### Example 1C: Synthesis of 5-Methyl-7-nitro-8-(4-((oxetan-3-ylmethyl)(phenyl)amino)piperidin-1-yl)-6-oxo-5,6-dihydro-1,5-naphthyridine-2-carbonitrile

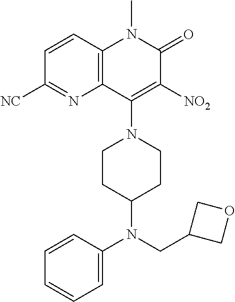 [SMILES: Cn1c(=O)c([N+](=O)[O-])c(N2CCC(N(CC3COC3)c3ccccc3)CC2)c2nc(C#N)ccc21]

### Step 1: tert-Butyl 4-((oxetan-3-ylmethyl)(phenyl)amino)piperidine-1-carboxylate

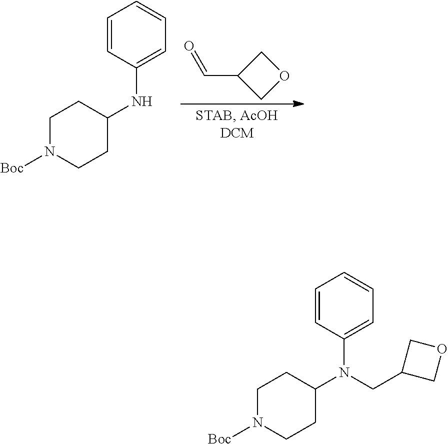

A solution of tert-butyl 4-(phenylamino) piperidine-1-carboxylate (600 mg, 2.17 mmol), oxetane-3-carbaldehyde (374 mg, 4.34 mmol), and acetic acid (0.05 mL) in DCM (5 mL) was stirred for 2 h at rt. Sodium triacetoxyborohydride (920 mg, 4.34 mmol) was added, and the resulting mixture was stirred for 16 h at rt. The residue was purified by reversed phase flash chromatography (Column: C18 silica gel, 80 g; Mobile phase, A: water (10 mM NH₄HCO₃) and ACN (5% to 100% in 30 min); Detector: UV 254 nm). The collected fraction was concentrated to afford the title compound (600 mg, 71%) as a yellow oil.

LCMS (ES, m/z): 347.20 [M+H]⁺.

### Step 2: N-(Oxetan-3-ylmethyl)-N-phenylpiperidin-4-amine trifluoroacetate

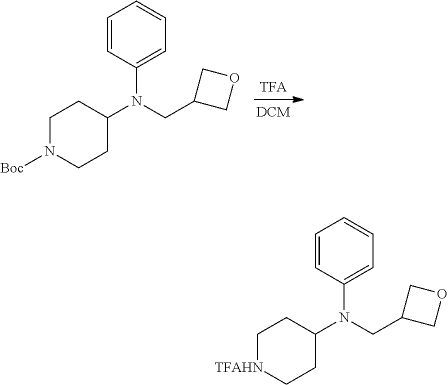

A solution of tert-butyl 4-((oxetan-3-ylmethyl)(phenyl)amino)piperidine-1-carboxylate (300 mg, 865 μmol) and TFA (0.5 mL) in DCM (2.5 mL) was stirred for 16 h at rt. The reaction was concentrated to afford the title compound (300 mg, assumed quantitative yield) as a yellow oil.

LCMS (ES, m/z): 247.10 [M+H]⁺.

### Step 3: 5-Methyl-7-nitro-8-(4-((oxetan-3-ylmethyl)(phenyl)amino)piperidin-1-yl)-6-oxo-5,6-dihydro-1,5-naphthyridine-2-carbonitrile

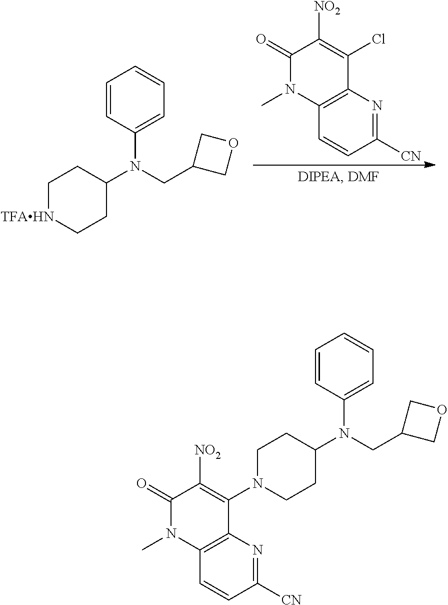

A solution methyl-7-nitro-6-oxo-1,5-naphthyridine-2-carbonitrile (100 mg, 378 μmol), N-(oxetan-3-ylmethyl)-N-phenylpiperidin-4-amine trifluoroacetate (93.1 mg, 378 μmol) and DIPEA (244 mg, 329 μL, 1.89 mmol) in DMF (4 mL) was stirred for 2 h at rt. The residue was purified by reversed phase flash chromatography (Column: C18, 80 g; Mobile phase, A: water (10 Mm NH₄HCO₃) and ACN (35% to 70% gradient in 30 min); Detector: UV 254 nm). The collected fraction was concentrated to afford the title compound (51 mg, 27%) as a yellow solid.

¹H NMR (400 MHz, DMSO-d₆) δ (ppm): 8.30 (d, J=8.8 Hz, 1H), 8.21 (d, J=9.2 Hz, 1H), 7.21-7.17 (m, 2H), 6.88 (d, J=8.0 Hz, 2H), 6.72 (t, J=7.2 Hz, 1H), 4.59-4.56 (m, 2H), 4.35-4.32 (m, 2H), 3.93-3.90 (m, 1H), 3.80-3.76 (m, 2H), 3.59 (s, 3H), 3.47 (d, J=6.8 Hz, 2H), 3.32-3.09 (m, 3H), 1.97-1.90 (m, 2H), 1.89-1.80 (m, 2H).

LCMS (ES, m/z): 475.15 [M+H]⁺.

### Example 1D: Synthesis of 5-Methyl-8-(4-(((3-methyloxetan-3-yl)methyl)(phenyl)amino) piperidin-1-yl)-7-nitro-6-oxo-5,6-dihydro-1,5-naphthyridine-2-carbonitrile

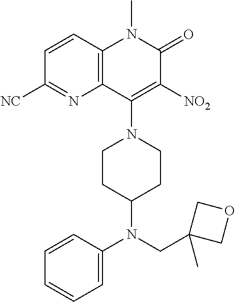 [SMILES: Cn1c(=O)c([N+](=O)[O-])c(N2CCC(N(CC3(C)COC3)c3ccccc3)CC2)c2nc(C#N)ccc21]

### Step 1: tert-Butyl 4-(((3-methyloxetan-3-yl)methyl)(phenyl)amino)piperidine-1-carboxylate

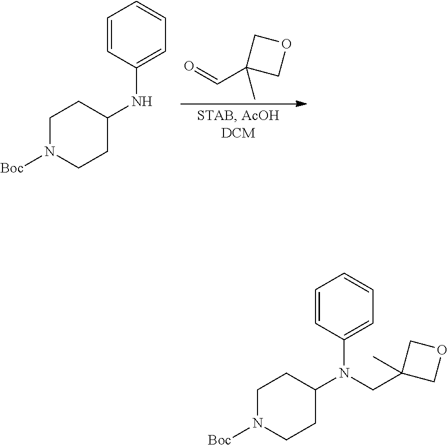

A mixture of tert-butyl 4-(phenylamino) piperidine-1-carboxylate (552 mg, 2.00 mmol), 3-methyloxetane-3-carbaldehyde (200 mg, 2.00 mmol) and acetic acid (1 drop) in DCM (4 mL) was stirred for 0.5 h at rt followed by addition of sodium triacetoxyborohydride (635 mg, 3.00 mmol). The resulting solution was stirred for 16 h at rt. The resulting mixture was concentrated, and the residue was purified by silica gel chromatography (Petroleum ether-ethyl acetate, 10:1) to afford tert-butyl 4-[N-[(3-methyloxetan-3-yl)methyl]anilino]piperidine-1-carboxylate (100 mg, 13%) as a white solid.

LCMS (ES, m/z): 361.15 [M+H]⁺.

### Step 2: N-((3-Methyloxetan-3-yl)methyl)-N-phenylpiperidin-4-amine hydrotrifluoroacetate

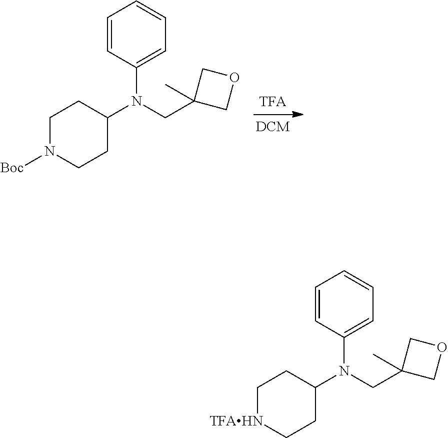

A solution of tert-butyl 4-(((3-methyloxetan-3-yl)methyl)(phenyl)amino)piperidine-1-carboxylate (90.0 mg, 250 μmol) in DCM (2.5 mL) and TFA (0.5 mL) was stirred for 1 h at rt. The resulting solution was concentrated under reduced pressure to afford the title compound (65 mg, assumed quantitative) as a yellow solid.

LCMS (ES, m/z): 261.15 [M+H]⁺.

### Step 3: 5-Methyl-8-(4-(((3-methyloxetan-3-yl)methyl)(phenyl)amino)piperidin-1-yl)-7-nitro-6-oxo-5,6-dihydro-1,5-naphthyridine-2-carbonitrile

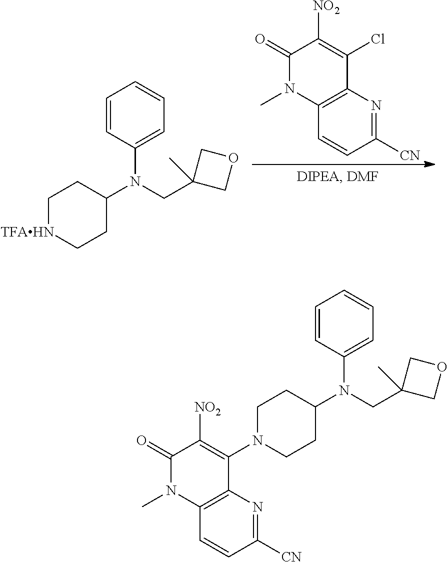

A mixture of N-((3-methyloxetan-3-yl)methyl)-N-phenylpiperidin-4-amine hydrotrifluoroacetate (55.0 mg, 211 μmol), 8-chloro-5-methyl-7-nitro-6-oxo-1,5-naphthyridine-2-carbonitrile (55.9 mg, 211 μmol) and DIPEA (81.9 mg, 634 μmol, 110 μL) in DMF (2 mL) was stirred for 1 h at rt. The resulting solution was purified by reversed phase flash chromatography (Column: C18 silica gel; Mobile phase, A: water (containing 10 mM NH₄HCO₃) and B: ACN (5% B to 75% B in 30 min); Detector: UV 254/220 nm). The collected fraction was lyophilized to afford the title compound (18.3 mg, 16%) as a yellow solid.

¹H-NMR (DMSO-d₆, 300 MHz) δ (ppm): 8.30 (d, J=8.7 Hz, 1H), 8.26 (d, J=8.7 Hz, 1H), 7.26 (t, J=7.2 Hz, 8.4 Hz, 2H), 6.96 (d, J=8.1 Hz, 2H), 6.82 (t, J=7.2 Hz, 1H), 4.39 (d, J=5.7 Hz, 2H), 3.98 (d, J=5.7 Hz, 2H), 3.85-3.68 (m, 3H), 3.58 (s, 3H), 3.34-3.14 (m, 4H), 1.92-1.75 (m, 4H), 1.34 (s, 3H).

LCMS (ES, m/z): 489.15 [M+H]⁺.

### Example 1E: Synthesis of 6-Chloro-4-(4-((cyclopropylmethyl)(2-hydroxyphenyl)amino) piperidin-1-yl)-1-methyl-2-oxo-1,2-dihydro-1,5-naphthyridine-3-carbonitrile

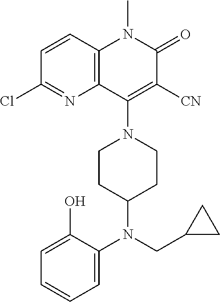 [SMILES: Cn1c(=O)c(C#N)c(N2CCC(N(CC3CC3)c3ccccc3O)CC2)c2nc(Cl)ccc21]

### Step 1: tert-Butyl 4-((2-methoxyphenyl)amino)piperidine-1-carboxylate

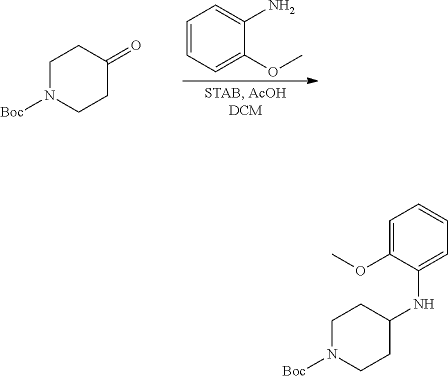

A solution of tert-butyl 4-oxopiperidine-1-carboxylate (8.20 g, 41.2 mmol), 2-methoxyaniline (4.61 g, 37.4 mmol, 4.23 mL) and acetic acid (112 mg, 1.87 mmol) in DCM (200 mL) was stirred for 0.5 h at rt. Sodium triacetoxyborohydride (15.8 g, 74.8 mmol) was added, and the resulting mixture was stirred for 12 h at rt. The mixture was poured into water (100 mL) and extracted with DCM (3×100 mL). The combined organic layers were washed with brine (100 mL), dried over anhydrous sodium sulfate and concentrated under reduced pressure. The residue was purified by silica gel chromatography (petroleum ether-ethyl acetate, 3:1) to afford the title compound (10.7 g, 93%) as a yellow solid.

LCMS (ES, m/z): 307.20 [M+H]⁺.

### Step 2: tert-Butyl 4-((cyclopropylmethyl)(2-methoxyphenyl)amino)piperidine-1-carboxylate

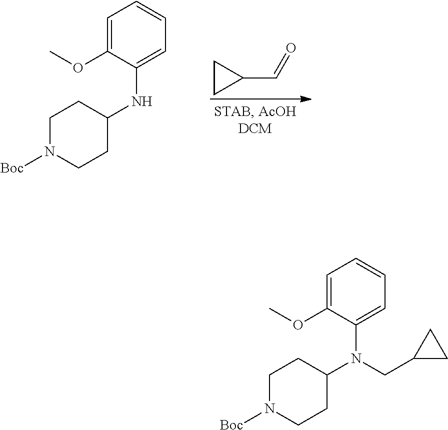 [SMILES: COc1ccccc1NC1CCN(C(=O)OC(C)(C)C)CC1 ; O=CC1CC1 ; COc1ccccc1N(CC1CC1)C1CCN(C(=O)OC(C)(C)C)CC1]

A solution of tert-butyl 4-((2-methoxyphenyl)amino)piperidine-1-carboxylate (5.00 g, 16.3 mmol) and cyclopropane carbaldehyde (2.29 g, 32.6 mmol, 2.44 mL) in DCM (40 mL) and acetic acid (80 mL) was stirred for 0.5 h at rt. Sodium triacetoxyborohydride (6.92 g, 32.6 mmol) was added, and the resulting mixture was stirred at rt for 1.5 h. The mixture was poured into water (100 mL) and extracted with DCM (3×100 mL). The combined organic layers were washed with brine (100 mL), dried over anhydrous sodium sulfate, and concentrated under reduced pressure. The residue was purified by silica gel chromatography with petroleum ether/ethyl acetate (10:1) to afford the title compound (1.7 g, 9%) as a yellow solid.

LCMS (ES, m/z): 361.30 [M+H]⁺.

### Step 3: 2-((Cyclopropylmethyl)(piperidin-4-yl)amino)phenol hydrobromide

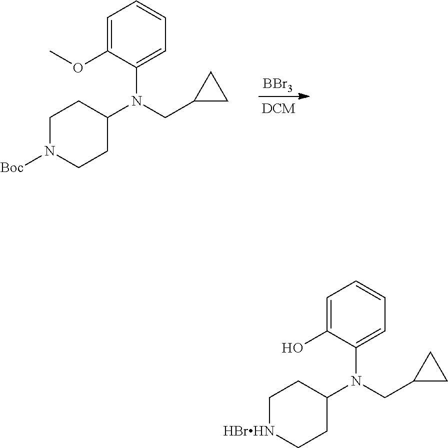

A solution of tert-butyl 4-((cyclopropylmethyl)(2-methoxyphenyl)amino)piperidine-1-carboxylate (500 mg, 1.39 mmol) and BBr₃ (1 M in DCM, 11.1 mL) in DCM (5 mL) was stirred for 12 h at rt. The mixture was poured into methanol (5 mL) and concentrated under reduced pressure to afford the title compound (580 mg, assumed quantitative yield) as a brown oil.

LCMS (ES, m/z): 247.20 [M+H]⁺.

### Step 4: 6-Chloro-4-(4-((cyclopropylmethyl)(2-hydroxyphenyl)amino)piperidin-1-yl)-1-methyl-2-oxo-1,2-dihydro-1,5-naphthyridine-3-carbonitrile

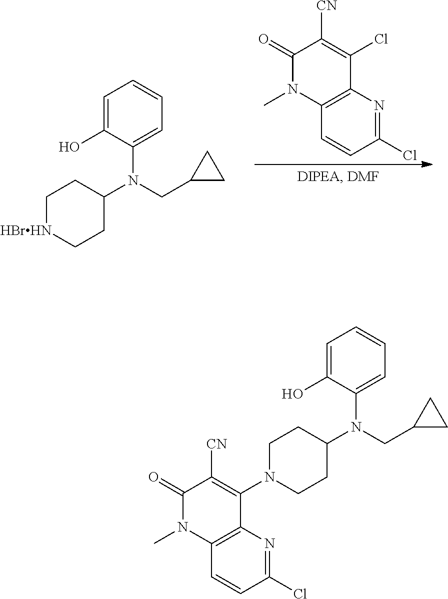

A solution of 2-((cyclopropylmethyl)(piperidin-4-yl)amino)phenol hydrobromide (87.0 mg, 353 μmol), 4,6-dichloro-1-methyl-2-oxo-1,5-naphthyridine-3-carbonitrile (116 mg, 455 μmol) and Cs₂CO₃ (445 mg, 1.37 mmol) in DMF (3 mL) was stirred for 2 h at 80° C. The mixture was cooled to rt, diluted with water (10 mL) and extracted with ethyl acetate (3×10 mL). The combined organic layers were washed with brine (30 mL), dried over anhydrous sodium sulfate, and concentrated under reduced pressure. The residue was purified by reversed phase chromatography (Column: Xselect CSH OBD, 30×150 mm, 5 um; Mobile Phase, A: water (10 mmol/L NH₄HCO₃+0.1% NH₃·H₂O) and B: ACN (55% to 75% in 7 min); Detector: 220 nm). The collected fraction was lyophilized to afford the title compound (8.3 mg, 4%) as a light-yellow solid. ¹H-NMR (DMSO-d₆, 300 MHz) δ (ppm): 8.34 (s, 1H), 8.04 (d, J=9.0 Hz, 1H), 7.77 (d, J=8.7 Hz, 1H), 7.21-7.19 (m, 1H), 6.99-6.96 (m, 1H), 6.83-6.75 (m, 2H), 4.22-4.17 (m, 2H), 3.50 (s, 3H), 3.44-3.36 (m, 3H), 2.87 (d, J=6.6 Hz, 2H), 2.00-1.96 (m, 2H), 1.72-1.60 (m, 2H), 0.78-0.65 (m, 1H), 0.28-0.21 (m, 2H), 0.00-0.09 (m, 2H).

LCMS (ES, m/z): 464.20 [M+H]⁺.

### Example 1F: Synthesis of N-(Cyclopropylmethyl)-1-(7-methyl-[1,2,4]triazolo[4,3-a]quinolin-5-yl)-N-phenylpiperidin-4-amine

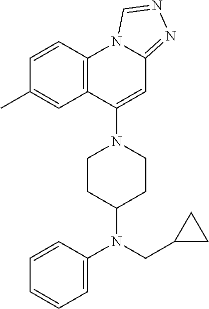 [SMILES: Cc1ccc2c(c1)c(N1CCC(N(CC3CC3)c3ccccc3)CC1)cc1nncn12]

### Step 1: 2,4-Dichloro-6-methylquinoline

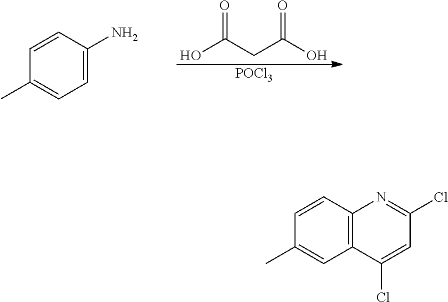

A solution of 4-methylaniline (10.0 g, 93.3 mmol, 10.3 mL) and malonic acid (9.71 g, 93.3 mmol) in phosphorus oxychloride (200 mL) was stirred for 5 h at 100° C. The resulting mixture was concentrated under reduced pressure and slowly poured onto ice with continuous stirring. The solid was filtered and extracted with ethyl acetate (3×300 mL). The combined organic layers were washed with brine (800 mL), dried over anhydrous sodium sulfate and concentrated under reduced pressure. The residue was purified by silica gel chromatography with petroleum ether/ethyl acetate (20:1) to afford the title compound (9.4 g, 48%) as a white solid.

LCMS (ES, m/z): 212.00 [M+H]⁺.

### Step 2: 4-Chloro-2-hydrazinyl-6-methylquinoline

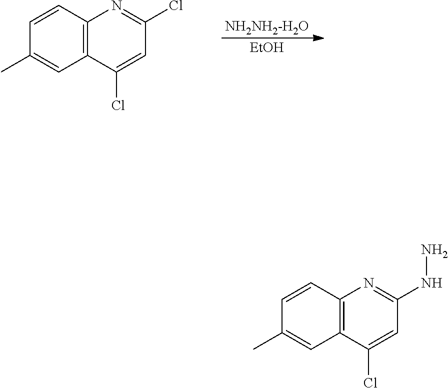

A solution of 2,4-dichloro-6-methylquinoline (21.0 g, 99.0 mmol) and hydrazine hydrate (282 g, 950 mmol, 275 mL) in ethanol (400 mL) was stirred for 16 h at 30° C. The resulting mixture was cooled to rt and partially concentrated under reduced pressure. The solution was extracted with DCM (3×100 mL), washed with brine (100 mL), dried over anhydrous sodium sulfate and concentrated under reduced pressure to afford the title compound (20.6 g, assumed quantitative) as a yellow solid.

LCMS (ES, m/z): 208.05 [M+H]⁺.

### Step 3: 5-Chloro-7-methyl-[1,2,4]triazolo[4,3-a]quinoline

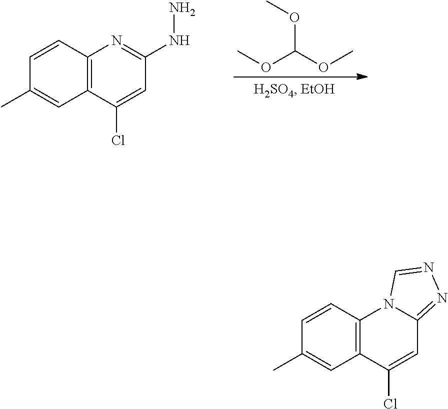

A solution of 4-chloro-2-hydrazinyl-6-methylquinoline (4.60 g, 22.2 mmol), trimethyl orthoformate (63.9 g, 602 mmol, 66 mL) and sulfuric acid (2.17 g, 22.2 mmol, 1 mL) in ethanol (400 mL) was stirred for 12 h at 45° C. The mixture was concentrated under reduced pressure and extracted with ethyl acetate (3×200 mL). The combined organic layers were washed with brine (500 mL), dried over anhydrous sodium sulfate and concentrated under reduced pressure. The residue was purified by reversed flash chromatography (Column: C18 silica gel, 120 g; Mobile Phase, A: water (0.1% NH₄HCO₃) and B: ACN (5% to 60% in 50 min); Detector: UV 254 nm) to afford the title compound (450 mg, assumed quantitative) as a light-yellow solid.

LCMS (ES, m/z): 218.00 [M+H]⁺.

### Step 4: tert-Butyl 4-((cyclopropylmethyl)(phenyl)amino)piperidine-1-carboxylate

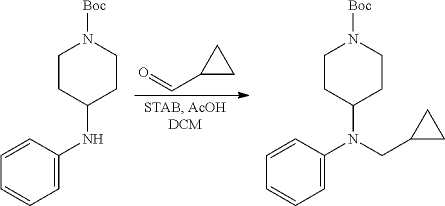

A solution of tert-butyl 4-(phenylamino)piperidine-1-carboxylate (2.79 g, 10.1 mmol) and cyclopropane carbaldehyde (1.06 g, 15.1 mmol, 1.13 mL) in acetic acid (10 mL) and DCM (20 mL) was stirred for 20 min at rt. Sodium triacetoxyborohydride (4.28 g, 20.2 mmol) was added, and the resulting mixture was stirred for 1 h at rt. The mixture was concentrated under reduced pressure and purified by reversed phase flash chromatography (Column: C18 silica gel; Mobile Phase, A: water (10 mM NH₄HCO₃) and B: ACN (0% to 80% in 30 min); Detector: UV 254 nm). The collected fraction was concentrated to afford the title compound (2.76 g, 81%).

LCMS (ES, m/z): 331.45 [M+H]⁺.

### Step 5: N-(Cyclopropylmethyl)-N-phenylpiperidin-4-amine hydrochloride

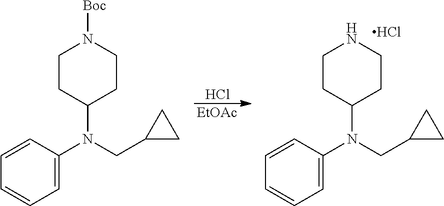 [SMILES: "EtOAc" ; CC(C)(C)OC(=O)N1CCC(N(CC2CC2)c2ccccc2)CC1 ; Cl ; c1ccc(N(CC2CC2)C2CCNCC2)cc1 ; Cl]

A solution of tert-butyl 4-((cyclopropylmethyl)(phenyl) amino)piperidine-1-carboxylate (1.30 g, 3.93 mmol) and HCl (4 M in dioxane, 9.83 mL) in ethyl acetate (15 mL) was stirred for 0.5 h at rt. The resulting mixture was concentrated under reduced pressure and washed with ethyl ether (15 mL). The solid was filtered to afford the title compound (1.04 g, 96%).

LCMS (ES, m/z): 231.20 [M+H]⁺.

### Step 6: N-(Cyclopropylmethyl)-1-(7-methyl-[1,2,4]triazolo[4,3-a]quinolin-5-yl)-N-phenylpiperidin-4-amine

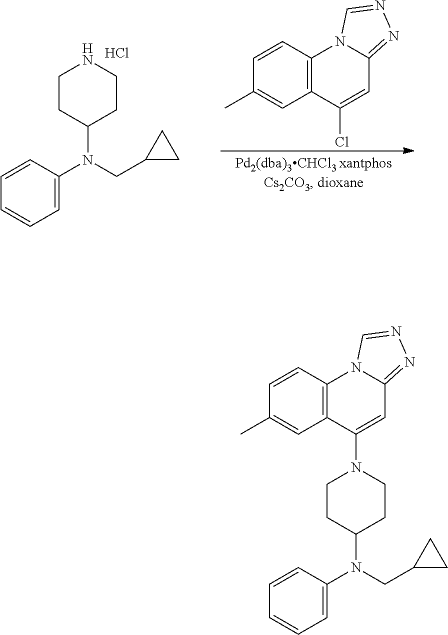 [SMILES: c1ccc(N(CC2CC2)C2CCNCC2)cc1 ; Cl ; Cc1ccc2c(c1)c(Cl)cc1nncn12 ; Cc1ccc2c(c1)c(N1CCC(N(CC3CC3)c3ccccc3)CC1)cc1nncn12]

A mixture of N-(cyclopropylmethyl)-N-phenylpiperidin-4-amine hydrochloride (160 mg, 695 μmol), 5-chloro-7-methyl-[1,2,4] triazolo[4,3-a] quinoline (151 mg, 694 μmol), Pd₂(dba)₃·CHCl₃ (72.0 mg, 69.5 μmol), (5-diphenylphosphanyl-9,9-dimethyl-xanthen-4-yl)-diphenyl-phosphane (81.0 mg, 139 μmol) and Cs₂CO₃ (679 mg, 2.08 mmol) in dioxane (5 mL) was stirred for 12 h at 110° C. under nitrogen atmosphere. The mixture was cooled to rt, the solids were filtered out, and the filtrate was concentrated under reduced pressure. The residue was purified by reversed phase chromatography (Column: XBridge Shield RP18 OBD, 19×250 mm, 10 um; Mobile Phase, A: water (10 mmol/L NH₄HCO₃+0.1% NH₃·H₂O) and B: ACN (67% to 80% in 7 min); Detector: 220 nm). The collected fraction was lyophilized to afford the title compound (20.5 mg, 7%).

¹H-NMR (DMSO-d₆, 300 MHz) δ (ppm): 9.77 (d, J=0.7 Hz, 1H), 8.31 (d, J=8.4 Hz, 1H), 7.82 (t, J=1.3 Hz, 1H), 7.61 (dd, J=8.5, 1.9 Hz, 1H), 7.27-7.15 (m, 2H), 7.08 (s, 1H), 6.94 (d, J=8.0 Hz, 2H), 6.66 (t, J=7.2 Hz, 1H), 3.92-3.79 (m, 1H), 3.49 (br d, J=11.7 Hz, 2H), 3.21 (d, J=5.8 Hz, 2H), 2.94-2.86 (m, 2H), 2.53 (s, 3H), 2.02 (dt, J=12.8, 9.2 Hz, 2H), 1.88 (br d, J=12.0 Hz, 2H), 1.05-0.95 (m, 1H), 0.54-0.50 (m, 2H), 0.36-0.31 (m, 2H).

LCMS (ES, m/z): 412.40 [M+H]⁺.

### Example 2

### Compounds of Structure (I-B-1)

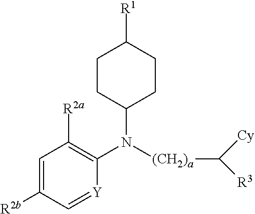

### Example 2A: trans-4-[4-[N-(Cyclopropylmethyl)anilino]cyclohexyl]-1-methyl-2-oxo-3H-pyrido[2,3-b]pyrazine-6-carbonitrile

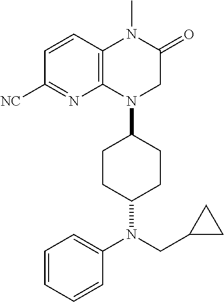 [SMILES: CN1C(=O)CN([C@H]2CC[C@H](N(CC3CC3)c3ccccc3)CC2)c2nc(C#N)ccc21]

### Step 1: trans-tert-Butyl N-[4-[N-(cyclopropylmethyl)anilino]cyclohexyl]carbamate

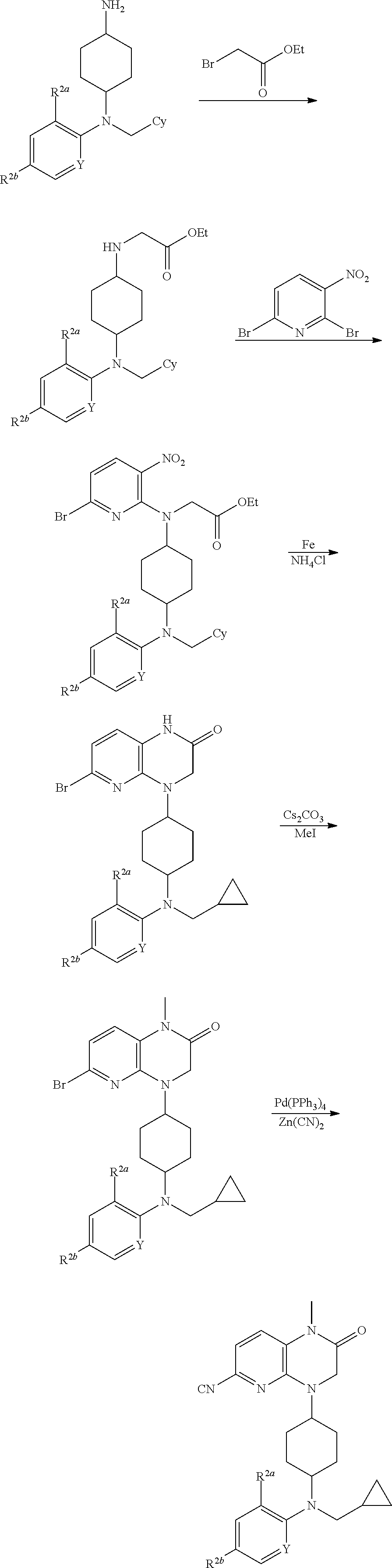

Prepared in a similar manner to Example 9, Step 2, using trans-tert-butyl N-(4-anilinocyclohexyl)carbamate (6.0 g, 20.7 mmol), cyclopropanecarbaldehyde (2.17 g, 31 mmol, 2.32 mL), STAB (8.76 g, 41.32 mmol), AcOH (30 mL) and DCM (60 mL) to afford the title compound (2.80 g, 8.13 mmol, 39%) as a light-yellow solid.

LCMS (ES, m/z): 345.25 [M+H]⁺

### Step 2: trans-N⁴-(Cyclopropylmethyl)-N⁴-phenyl-cyclohexane-1,4-diamine hydrotrifluoroacetate

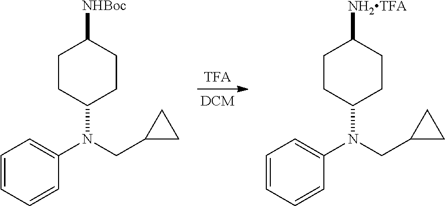

A solution of trans-tert-butyl N-[4-[N-(cyclopropylmethyl)anilino]cyclohexyl]carbamate (2.8 g, 8.13 mmol) in DCM (20 mL) and TFA (5 mL) was stirred at room temperature for 2 h. The mixture was concentrated under reduced pressure to afford the title compound (3 g, assumed quantitative) as a brown oil.

LCMS (ES, m/z): 245.20 [M-TFA+H]⁺

### Step 3: trans-2-[[4-[N-(Cyclopropylmethyl)anilino]cyclohexyl]amino]acetate

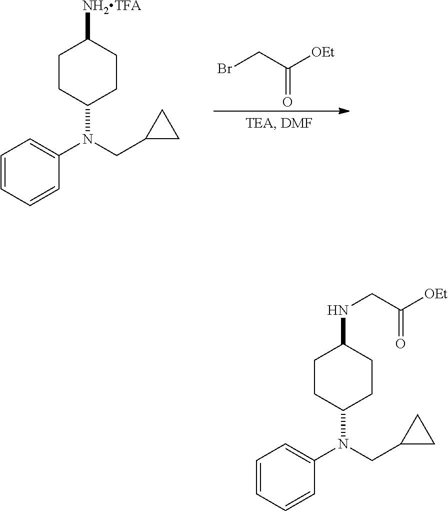

A solution of trans-N⁴-(cyclopropylmethyl)-N⁴-phenyl-cyclohexane-1,4-diamine hydrotrifluoroacetate (2 g, 5.86 mmol) and TEA (8.28 g, 81.8 mmol, 11.41 mL), Cs₂CO₃ (20.60 g, 24.55 mmol) in DMF (25 mL) was stirred at 50° C. for 48 h. The mixture was poured into water (50 mL) and extracted with EtOAc (3×50 mL). The combined organic layers were washed with brine (50 mL), dried over anhydrous sodium sulfate and concentrated under reduced pressure. The crude residue was purified by silica gel chromatography (Petroleum ether-EtOAc, 2:3) to afford the title compound (1.1 g, 3.33 mmol, 41%) as a brown oil.

LCMS (ES, m/z): 331.25 [M+H]⁺

### Step 4: trans-ethyl 2-[(6-Bromo-3-nitro-2-pyridyl)-[4-[N-(cyclopropylmethyl)anilino]-cyclohexyl]amino]acetate

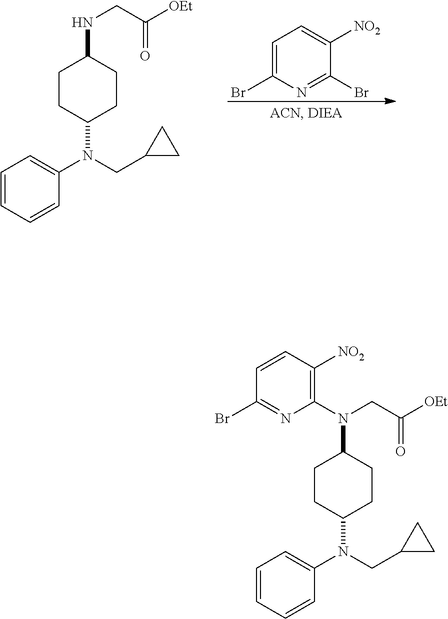

A solution of ethyl trans-2-[[4-[N-(cyclopropylmethyl)anilino]cyclohexyl]amino]acetate (970 mg, 2.94 mmol), 2,6-dibromo-3-nitro-pyridine (993 mg, 3.52 mmol) and DIEA (1.14 g, 8.81 mmol) in ACN (15 mL) was stirred at 80° C. for 2 h. The mixture was then cooled to rt, diluted with water (50 mL) and extracted with EtOAc (3×40 mL). The combined organic layers were washed with brine (50 mL), dried over anhydrous sodium sulfate and concentrated under reduced pressure. The crude residue was purified by silica gel chromatography (petroleum ether-EtOAc, 10:1) to afford the title compound (1.5 g, 2.82 mmol, 96%) as a yellow solid. LCMS (ES, m/z): 531.20, 533.20 [M+H]⁺

### Step 5: trans-6-Bromo-4-[4-[N-(cyclopropylmethyl)anilino]cyclohexyl]-1,3-dihydropyrido[2,3-b]pyrazin-2-one

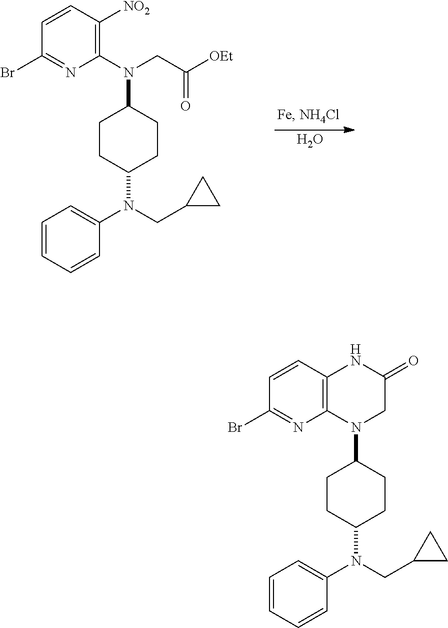

A mixture of trans-ethyl 2-[(6-bromo-3-nitro-2-pyridyl)-[4-[N-(cyclopropylmethyl) anilino]-cyclohexyl]amino]acetate (1.5 g, 2.82 mmol), Fe (788 mg, 14.11 mmol) and NH₄Cl (453 mg, 8.47 mmol) in THE (6 mL), H₂O (2 mL) and EtOH (6 mL) was stirred at 70° C. for 12 h. After cooling to rt, the solids were filtered off and the filtrate was concentrated. The crude residue was purified by silica gel chromatography (petroleum ether-EtOAc, 1:1) to afford the title compound (970 mg, 2.13 mmol, 75%) as a white solid.

LCMS (ES, m/z): 455.20, 457.20 [M+H]⁺

### Step 6: trans-6-Bromo-4-[4-[N-(cyclopropylmethyl)anilino]cyclohexyl]-1-methyl-3H-pyrido[2,3-b]pyrazin-2-one

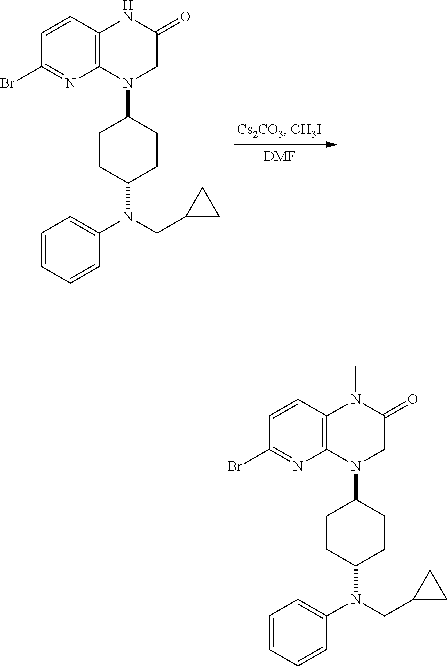

A slurry of trans-6-bromo-4-[4-[N-(cyclopropylmethyl)anilino]cyclohexyl]-1,3-dihydropyrido[2,3-b]pyrazin-2-one (1 g, 2.20 mmol), Cs₂CO₃ (2.13 g, 6.59 mmol) and CH₃I (3.12 g, 22.0 mmol) in DMF (20 mL) was stirred for 3 h. The mixture was diluted with water (50 mL) and extracted with EtOAc (3×40 mL). The combined organic layers were washed with brine (30 mL), dried over anhydrous sodium sulfate and concentrated under reduced pressure. The crude residue was purified by silica gel chromatography (petroleum ether-EtOAc, 1:1) to afford the title compound (970 mg, 2.07 mmol, 94%) as a white solid.

LCMS (ES, m/z): 469.15, 471.15 [M+H]⁺

### Step 7: trans-4-[4-[N-(Cyclopropylmethyl)anilino]cyclohexyl]-1-methyl-2-oxo-3H-pyrido[2,3-b]pyrazine-6-carbonitrile

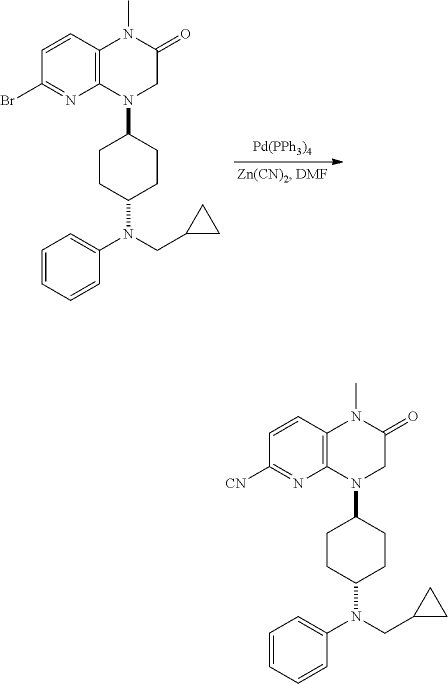

A solution of trans-6-bromo-4-[4-[N-(cyclopropylmethyl)anilino]cyclohexyl]-1-methyl-3H-pyrido[2,3-b]pyrazin-2-one (200 mg, 426 μmol), Zn(CN)₂ (37.5 mg, 319.6 μmol) and Pd(PPh₃)₄ (15.7 mg, 42.6 μmol) in DMF (3 mL) was heated to 100° C. for 2 h under N₂ atmosphere. After cooling to rt, it was diluted with water (10 mL) and extracted with EtOAc (3×10 mL). The combined organic layers were washed with brine (20 mL), dried over anhydrous sodium sulfate and concentrated under reduced pressure. The crude residue was purified by HPLC (Column: YMC-Actus Triart C18, 30 mm×150 mm, 5 um; Mobile Phase, A: water (10 mmol/L NH₄HCO₃+0.1% NH₃·H₂O) and B: ACN (67% to 87% in 7 min); Detector: 254 nm), to afford the title compound (45 mg, 108 μmol, 25%) as a white solid.

¹H NMR (400 MHz, CDCl₃) δ 7.29-7.21 (m, 2H), 7.08 (d, J=7.9 Hz, 1H), 6.99 (d, J=7.9 Hz, 1H), 6.88 (d, J=8.4 Hz, 2H), 6.73 (t, J=7.2 Hz, 1H), 4.67-4.54 (m, 1H), 4.09 (s, 2H), 3.64-3.53 (m, 1H), 3.34 (s, 3H), 3.12 (d, J=5.7 Hz, 2H), 2.05-1.98 (m, 2H), 1.91-1.83 (m, 2H), 1.76-1.59 (m, 4H), 1.06-0.99 (m, 1H), 0.59-0.52 (m, 2H), 0.32-0.25 (m, 2H).

LCMS (ES, m/z): 469.15, 471.15 [M+H]⁺

### Example 2B: 4-(trans-4-(Cyclopropyl(2-hydroxyphenyl)amino)cyclohexyl)-1-methyl-2-oxo-1,2,3,4-tetrahydropyrido[3,2-b]pyrazine-6-carbonitrile

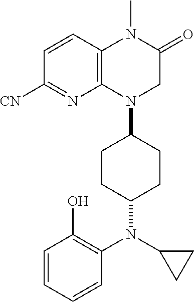

### Step 1: trans-ethyl 2-(4-(Cyclopropyl(2-hydroxyphenyl)amino)cyclohexylamino)acetate

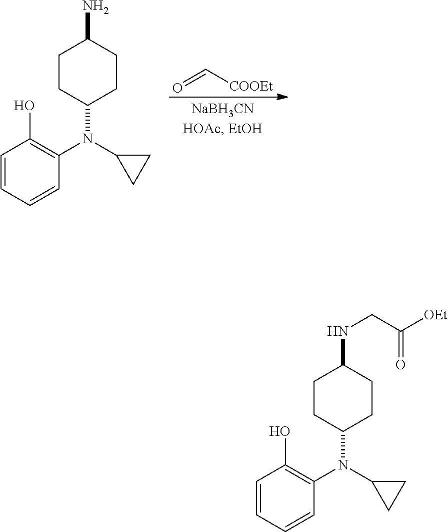

To a solution of trans-2-[(4-aminocyclohexyl)-cyclopropyl-amino]phenol (9.0 g, 36.5 mmol) and ethyl 2-oxoacetate in toluene (14.48 mL, 73.07 mmol, 50%) in EtOH (80 mL) and AcOH (5 mL) was added NaBH₃CN (15.94 g, 36.53 mmol). After stirring for 18 h, the mixture was diluted with water (100 mL) and extracted with EtOAc (3×100 mL). The combined organic layers were washed with brine (100 mL), dried over anhydrous sodium sulfate and concentrated under reduced pressure to afford (9.43 g, 37%) as a light yellow oil.

LCMS (ES, m/z): 333.16 [M+H]⁺

### Step 2: trans-Ethyl 2-((6-bromo-3-nitropyridin-2-yl)(4-(cyclopropyl(2-hydroxyphenyl) amino)-cyclohexyl)amino)acetate

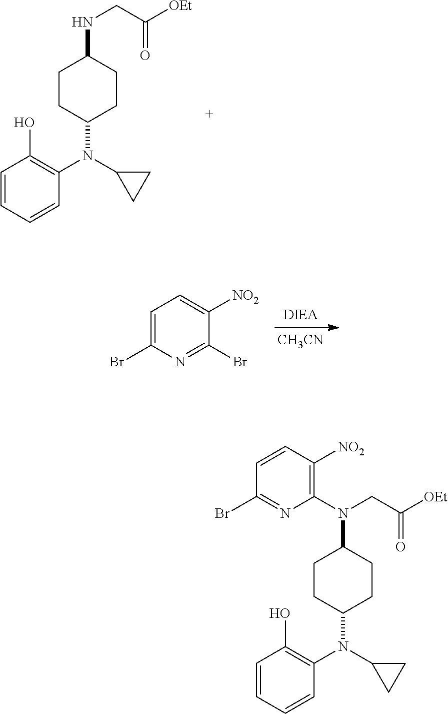

A solution of 2,6-dibromo-3-nitro-pyridine (8.0 g, 28.4 mmol), ethyl 2-(trans-4-(cyclopropyl(2-hydroxyphenyl)amino)cyclohexylamino)acetate (9.43 g, 28.38 mmol) and DIEA (18.34 g, 141.90 mmol) in ACN (50 mL) was stirred at 80° C. for 1 h, whereupon the mixture was cooled to rt, diluted with water (100 mL) and extracted with EtOAc (3×100 mL). The combined organic layers were washed with brine (100 mL), dried over anhydrous sodium sulfate and concentrated under reduced pressure. The crude residue purified by silica gel chromatography (petroleum ether-EtOAc, 2:1) to afford the title compound (1.3 g, 8%) as a yellow oil.

LCMS (ES, m/z): 533.12, 535.12 [M+H]⁺

### Step 3: trans-Ethyl 2-((6-bromo-3-nitropyridin-2-yl)(4-(cyclopropyl(2-((2-(trimethylsilyl)ethoxy) methoxy)phenyl)amino)cyclohexyl)amino)acetate

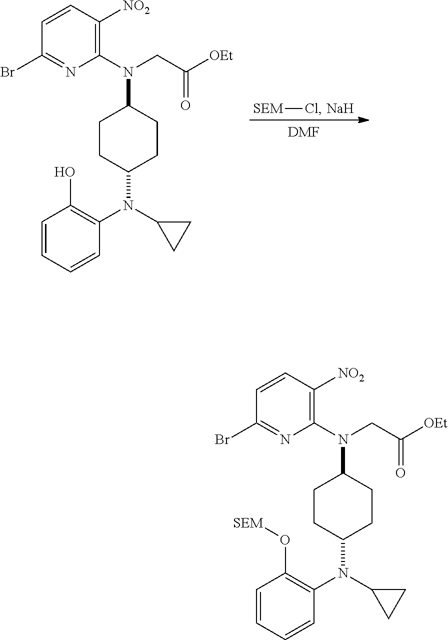

To a solution of trans-2-((6-bromo-3-nitropyridin-2-yl)(4-(cyclopropyl(2-hydroxyphenyl) amino)cyclohexyl)amino)acetate (1.07 g, 2.00 mmol) in DMF (20 mL) at 0° C. was added NaH (150 mg, 3.75 mmol, 60% in min. oil). After 30 min at 0° C., SEM-Cl (625.1 mg, 3.75 mmol) was added then allowed to warm to rt. After 2 h at rt, the mixture was diluted with saturated NH₄Cl (30 mL) and extracted with EtOAc (3×30 mL). The combined organic layers were washed with brine (50 mL), dried over anhydrous sodium sulfate and concentrated under reduced pressure to afford the title compound (1.3 g, 97%) as a black oil. LCMS (ES, m/z): 663.15, 665.15 [M+H]⁺

### Step 4: trans-6-Bromo-4-(4-(cyclopropyl(2-((2-(trimethylsilyl)ethoxy)methoxy)phenyl) amino)-cyclohexyl)-3,4-dihydropyrido[2,3-b]pyrazin-2(1H)-one

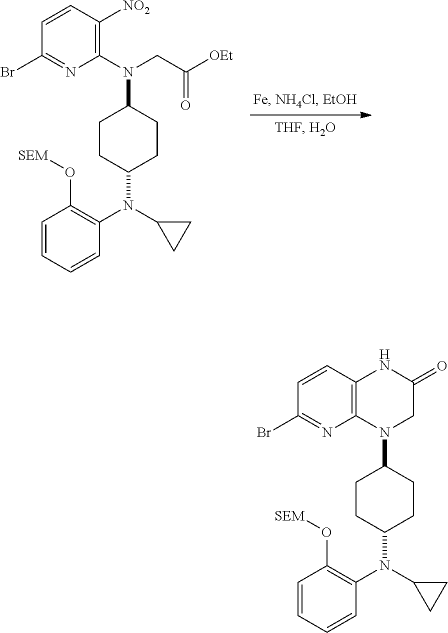

A solution of trans-ethyl 2-((6-bromo-3-nitropyridin-2-yl)(4-(cyclopropyl(2-((2-(trimethylsilyl)ethoxy)methoxy)phenyl)amino)cyclohexyl)amino)acetate (0.90 g, 1.37 mmol), Fe (383 mg, 6.86 mmol), NH₄Cl (220 mg, 4.11 mmol) in EtOH (10 mL), H₂O (3 mL) and THF (10 mL) was stirred at 70° C. for 3 h. After cooling to rt, the solids were filtered off and the filtrate was diluted with water (30 mL) then extracted with EtOAc (3×30 mL). The combined organic layers were washed with brine (30 mL), dried over anhydrous sodium sulfate and concentrated under reduced pressure. The crude residue was purified by silica gel chromatography (petroleum ether-EtOAc, 2:1) to afford the title compound (300 mg, 33%) as light yellow solid. LCMS (ES, m/z): 587.16, 589.16 [M+H]⁺

### Step 5: trans-6-Bromo-4-(4-(cyclopropyl(2-((2-(trimethylsilyl)ethoxy)methoxy)phenyl)-amino)cyclohexyl)-1-methyl-3,4-dihydropyrido[2,3-b]pyrazin-2(1H)-one

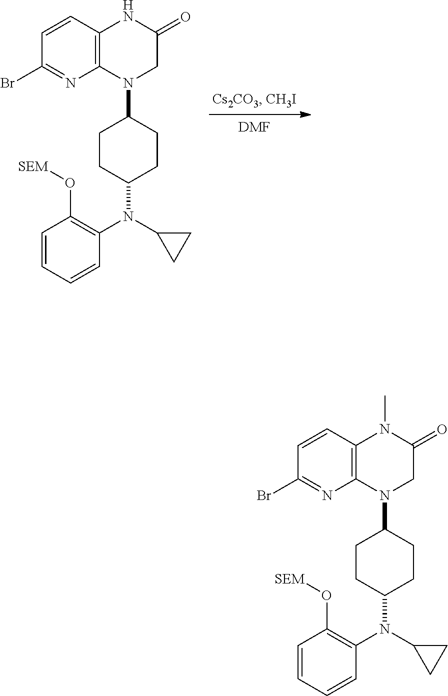

To a slurry of 6-bromo-4-(trans-4-(cyclopropyl(2-((2-(trimethylsilyl)ethoxy)methoxy) phenyl)-amino)cyclohexyl)-3,4-dihydropyrido[2,3-b]pyrazin-2(1H)-one (290 mg, 493.5 μmol) and Cs₂CO₃ (320 mg, 987 μmol) in DMF (8 mL) was added CH₃I (140 mg, 987 μmol). After stirring for 2 h, it was diluted with saturated NH₄Cl (aq, 20 mL) then extracted with EtOAc (3×20 mL). The combined organic layers were washed with brine (30 mL), dried over anhydrous sodium sulfate and concentrated under reduced pressure to afford the title compound (280 mg, 88%) as a black oil. LCMS (ES, m/z): 601.16, 603.16 [M+H]⁺

### Step 6: trans-4-(4-(Cyclopropyl(2-((2-(trimethylsilyl)ethoxy)methoxy)phenyl)amino)-cyclohexyl)-1-methyl-2-oxo-1,2,3,4-tetrahydropyrido[3,2-b]pyrazine-6-carbonitrile

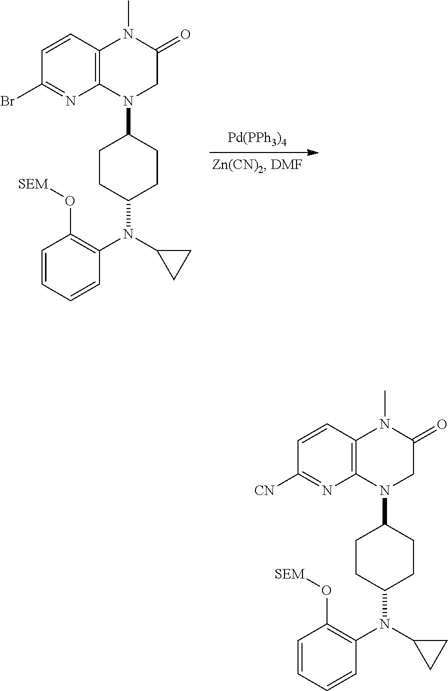

A solution of trans-6-bromo-4-(4-(cyclopropyl(2-((2-(trimethylsilyl)ethoxy)methoxy) phenyl)-amino)cyclohexyl)-1-methyl-3,4-dihydropyrido[2,3-b]pyrazin-2(1H)-one (280 mg, 465 μmol), Zn(CN)₂ (110 mg, 931 μmol), Pd(PPh₃)₄ (108 mg, 93.08 μmol) in DMF (10 mL) was heated to 100° C. After 1 h, it was cooled to rt, diluted with water (30 mL) and extracted with EtOAc (3×30 mL). The combined organic layers were washed with brine (30 mL), dried over anhydrous sodium sulfate and concentrated under reduced pressure. The crude residue was purified via silica gel chromatography (petroleum ether-EtOAc, 2:1) to afford the title compound (200 mg, 74%) as a yellow oil. LCMS (ES, m/z): 548.32 [M+H]⁺

### Step 7: trans-4-(4-(Cyclopropyl(2-hydroxyphenyl)amino)cyclohexyl)-1-methyl-2-oxo-1,2,3,4-tetrahydropyrido[3,2-b]pyrazine-6-carbonitrile

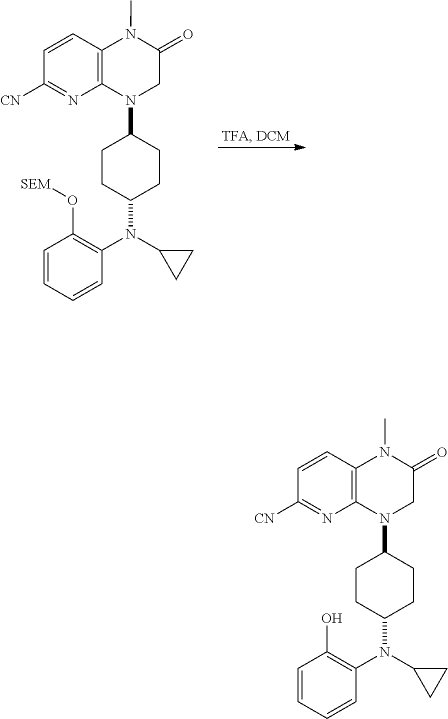

To a solution of trans-4-(4-(cyclopropyl(2-((2-(trimethylsilyl)ethoxy)methoxy)phenyl) amino)-cyclohexyl)-1-methyl-2-oxo-1,2,3,4-tetrahydropyrido[3,2-b]pyrazine-6-carbonitrile (100 mg, 183 μmol) in DCM (3 mL) was added TFA (1 mL). After stirring for 1 h, it was concentrated under reduced pressure and the crude residue was purified by HPLC (Column: XBridge Prep Phenyl OBD, 5 um, 19×250 mm; Mobile Phase, A: Water (0.05% TFA) and B: ACN (40% to 60% in 7 min); Detector: 220 nm), affording the title compound (6.7 mg, 8%) as a light brown solid. ¹H NMR (300 MHz, CDCl₃) δ 7.14-7.02 (m, 2H), 7.02-6.86 (m, 4H), 4.57-4.45 (m, 1H), 4.01 (s, 2H), 3.32 (s, 3H), 2.97-2.83 (m, 1H), 1.83-1.75 (m, 2H), 1.69-1.42 (m, 5H), 0.91-0.79 (m, 3H), 0.65-0.50 (m, 2H). LCMS (ES, m/z): 418.15 [M+H]⁺

### Example 2C: cis-4-[4-[N-(Cyclopropylmethyl)anilino]cyclohexyl]-1-methyl-2-oxo-3H-pyrido[2,3-b]pyrazine-6-carbonitrile

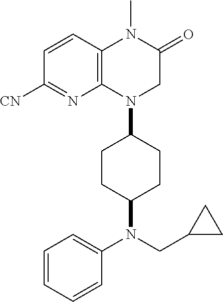

Prepared in an analogous manner to Example 33. The crude material was purified by reverse flash chromatography (Column: C18 silica gel, 80 g, 20-35 um; mobile phase, A: water with 10% NH₄HCO₃ and B: ACN, 5-100% over 50 min), affording the title compound (65.5 mg, 156 μmol, 24.44%) as a white solid.

¹H NMR (400 MHz, CDCl₃) δ 7.32-7.26 (m, 2H), 7.20-7.16 (m, 2H), 7.07-7.02 (m, 2H), 6.97 (d, J=7.9 Hz, 1H), 4.70-4.60 (m, 1H), 4.15 (s, 2H), 3.59-3.55 (m, 1H), 3.34 (s, 3H), 2.93 (d, J=6.6 Hz, 2H), 2.09-1.99 (m, 2H), 1.98-1.90 (m, 2H), 1.66-1.57 (m, 2H), 1.42-1.35 (m, 2H), 0.80-0.70 (m, 1H), 0.35-0.30 (m, 2H), −0.07-(−) 0.15 (m, 2H).

LCMS (ES, m/z): 416.20 [M+H]⁺.

### Example 3

### Compounds of Structure (I-B-2) or (I-B-3)

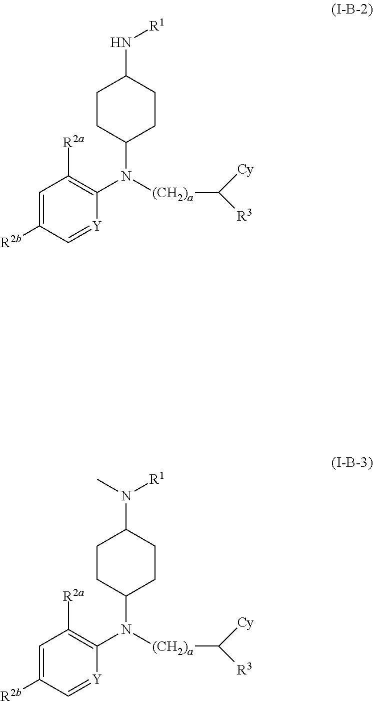

### Example 3A: Synthesis of trans-6-Chloro-4-((4-(cyclopropyl(phenyl)amino)cyclohexyl)amino)-1-methyl-2-oxo-1,2-dihydro-1,5-naphthyridine-3-carbonitrile

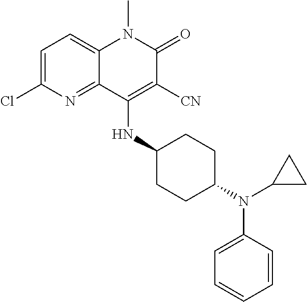 [SMILES: Cn1c(=O)c(C#N)c(N[C@H]2CC[C@H](N(c3ccccc3)C3CC3)CC2)c2nc(Cl)ccc21]

### Step 1: trans-tert-Butyl (4-(cyclopropyl(phenyl)amino)cyclohexyl)carbamate

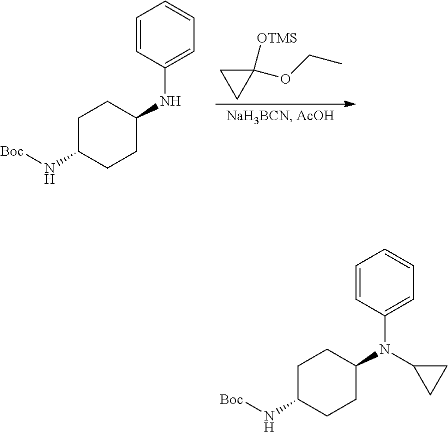

A solution of trans-tert-butyl (4-(phenylamino)cyclohexyl)carbamate (700 mg, 2.41 mmol), (1-ethoxycyclopropoxy)trimethylsilane (2.10 g, 12.1 mmol, 2.42 mL), sodium cyanoborohydride (606 mg, 9.64 mmol) and 3 AMS (487 mg) in acetic acid (20 mL) was stirred for 2 h at 70° C. under nitrogen atmosphere. The mixture was cooled to rt, diluted with water (40 mL) and extracted with ethyl acetate (3×40 mL). The combined organic layers were washed with brine (100 mL), dried over anhydrous sodium sulfate, and concentrated under reduced pressure. The residue was purified by silica gel chromatography with petroleum ether/ethyl acetate (5:1) to afford the title compound (220 mg, 27%) as a white solid.

LCMS (ES, m/z): 331.45 [M+H]⁺.

### Step 2: N-Cyclopropyl-N-phenylcyclohexane-1,4-diamine hydrotrifluoroacetate

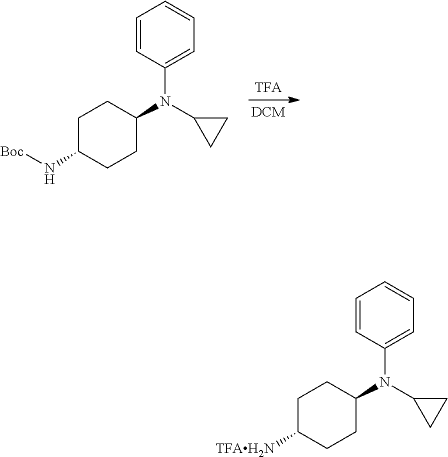

A solution of trans-tert-butyl (4-(cyclopropyl(phenyl)amino)cyclohexyl)carbamate (200 mg, 605 μmol) in DCM (2 mL) and TFA (0.5 mL) was stirred for 2 h at rt. The resulting mixture was concentrated under reduced pressure to afford the title compound (450 mg, assumed quantitative) as a brown oil.

LCMS (ES, m/z): 231.55 [M+H]⁺.

### Step 3: trans-6-Chloro-4-((4-(cyclopropyl(phenyl)amino)cyclohexyl)amino)-1-methyl-2-oxo-1,2-dihydro-1,5-naphthyridine-3-carbonitrile

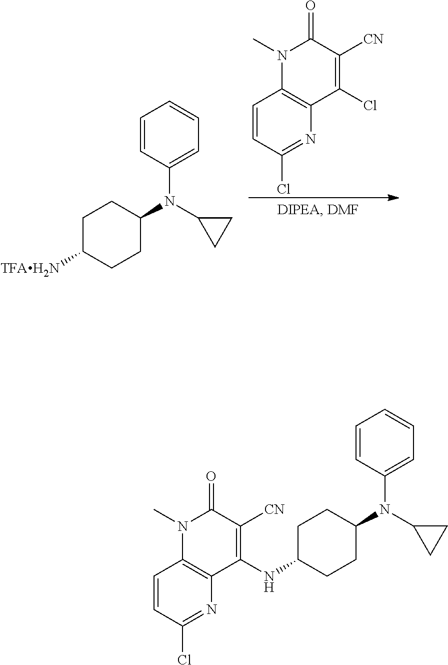

A solution of N-cyclopropyl-N-phenylcyclohexane-1,4-diamine hydrotrifluoroacetate (60 mg, 260 μmol), 4,6-dichloro-1-methyl-2-oxo-1,5-naphthyridine-3-carbonitrile (68.0 mg, 268 μmol) and DIPEA (180 mg, 1.39 mmol, 242 μL) in DMF (3 mL) was stirred for 2 h at rt. The resulting mixture was purified by reversed phase flash chromatography (Column: C18 silica gel, 80 g; Mobile Phase, A: water (0.05% NH₄HCO₃) and B: ACN (0% to 100% in 30 min); Detector: UV 254 nm). The collected fraction was lyophilized to afford the title compound (34.9 mg, 28%) as a light-yellow solid.

¹H-NMR (DMSO-d₆, 300 MHz) δ (ppm): 8.03 (d, J=3.3 Hz, 1H), 7.87 (d, J=3.9 Hz, 1H), 7.62 (d, J=9 Hz, 1H), 7.23-7.18 (m, 2H), 7.03 (d, J=7.5 Hz, 2H), 6.79-6.75 (m, 1H), 4.32-4.25 (m, 1H), 3.57-3.52 (m, 1H), 3.49 (s, 3H), 2.49-2.33 (m, 1H), 2.14-2.08 (m, 2H), 1.87-1.65 (m, 6H), 0.84-0.78 (m, 2H), 0.40-0.35 (m, 2H).

LCMS (ES, m/z): 448.30 [M+H]⁺.

### Example 3B: Synthesis of trans-6-Chloro-4-((4-((cyclopropylmethyl)(phenyl)amino) cyclohexyl) amino)-1-methyl-2-oxo-1,2-dihydro-1,5-naphthyridine-3-carbonitrile

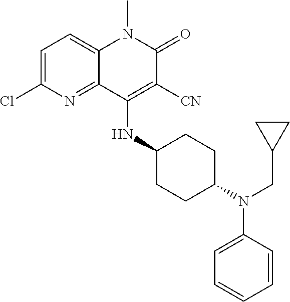 [SMILES: Cn1c(=O)c(C#N)c(N[C@H]2CC[C@H](N(CC3CC3)c3ccccc3)CC2)c2nc(Cl)ccc21]

### Step 1: trans-tert-Butyl (4-(phenylamino)cyclohexyl)carbamate

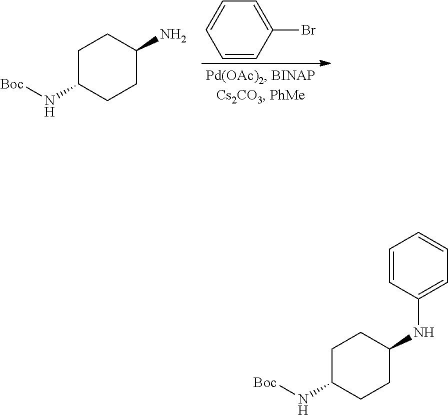

A solution of trans-tert-butyl (4-aminocyclohexyl)carbamate (5.00 g, 23.3 mmol), bromobenzene (7.33 g, 46.7 mmol), BINAP (2.91 g, 4.67 mmol), dicesium carbonate (15.2 g, 46.7 mmol) and palladium acetate (524 mg, 2.33 mmol) in toluene (100 mL) was stirred for 24 h at 110° C. under nitrogen atmosphere. The mixture was cooled to rt, diluted with water (200 mL) and extracted with ethyl acetate (3×150 mL). The combined organic layers were washed with brine (400 mL), dried over anhydrous sodium sulfate, and concentrated under reduced pressure. The residue was purified by silica gel chromatography (Petroleum ether-ethyl acetate, 3:1) to afford the title compound (1.10 g, 16%) as a yellow solid.

LCMS (ES, m/z): 291.20 [M+H]⁺.

### Step 2: trans-tert-Butyl (4-((cyclopropylmethyl)(phenyl)amino)cyclohexyl)carbamate

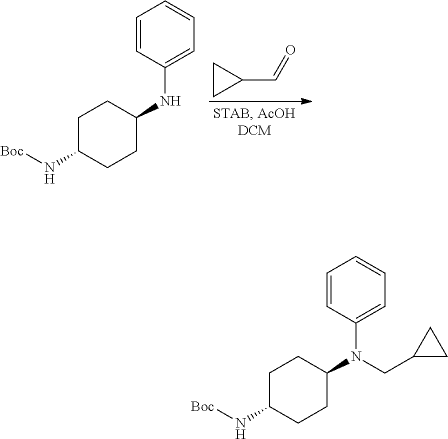

A solution of trans-tert-butyl (4-(phenylamino)cyclohexyl)carbamate (300 mg, 1.03 mmol) and cyclopropane carbaldehyde (108 mg, 1.55 mmol, 116 μL) in DCM (4 mL) and acetic acid (0.8 mL) was stirred for 0.5 h at rt. Sodium triacetoxyborohydride (438 mg, 2.07 mmol) was added, and the resulting mixture was stirred for 1.5 h at rt. The mixture was poured into ice/water (10 mL) and extracted with DCM (3×10 mL). The combined organic layers were washed with brine (30 mL), dried over anhydrous sodium sulfate, and concentrated under reduced pressure. The residue was purified by silica gel chromatography (Petroleum ether-ethyl acetate, 3:1) to afford the title compound (270 mg, 75%) as a yellow solid.

LCMS (ES, m/z): 345.25 [M+H]⁺.

### Step 3: trans-N-(Cyclopropylmethyl)-N-phenylcyclohexane-1,4-diamine hydrotrifluoroacetate

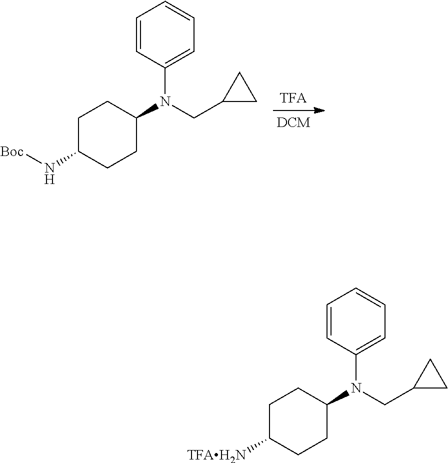

A solution of trans-tert-butyl (4-((cyclopropylmethyl)(phenyl)amino)cyclohexyl) carbamate (260 mg, 754 μmol) in TFA (1 mL) and DCM (10 mL) was stirred for 2 h at rt. The mixture was concentrated under reduced pressure to afford the title compound (250 mg, assumed quantitative yield) as a brown oil.

LCMS (ES, m/z): 245.20 [M+H]⁺.

### Step 4: trans-6-Chloro-4-((4-((cyclopropylmethyl)(phenyl)amino)cyclohexyl)amino)-1-methyl-2-oxo-1,2-dihydro-1,5-naphthyridine-3-carbonitrile

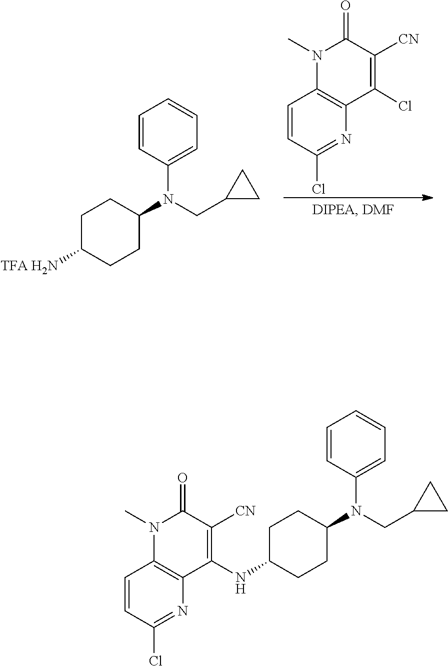

A solution of trans-N-(cyclopropylmethyl)-N-phenylcyclohexane-1,4-diamine trifluoroacetate (96.2 mg, 393 μmol), 4,6-dichloro-1-methyl-2-oxo-1,5-naphthyridine-3-carbonitrile (100 mg, 393 μmol) and DIPEA (254 mg, 1.97 mmol) in DMF (3 mL) was stirred for 2 h at rt. The mixture was poured into ice/water (10 mL) and extracted with ethyl acetate (3×10 mL). The combined organic layers were washed with brine (30 mL), dried over anhydrous sodium sulfate, and concentrated under reduced pressure. The residue was purified by reversed phase chromatography (Column: Xselect CSH OBD, 30×150 mm, 5 um; Mobile Phase, A: Water (10 mmol/L NH₄HCO₃+0.1% NH₃·H₂O) and B: ACN (67% to 87% in 7 min); Detector: 254 nm). The collected fraction was lyophilized to afford the title compound (21.7 mg, 12%) as a light-yellow solid.

¹H-NMR (DMSO-d₆, 300 MHz) δ (ppm): 8.04 (d, J=9.0 Hz, 1H), 7.85 (d, J=9.0 Hz, 1H), 7.61 (d, J=8.7 Hz, 1H), 7.21-7.16 (m, 2H), 6.84 (d, J=8.1 Hz, 2H), 6.65-6.60 (m, 1H), 4.40-4.15 (m, 1H), 3.76-3.60 (m, 1H), 3.39 (s, 3H), 3.07 (d, J=5.4 Hz, 2H), 2.22-2.08 (m, 2H), 1.96-1.70 (m, 4H), 1.70-1.50 (m, 2H), 1.02-0.82 (m, 1H), 0.58-0.40 (m, 2H), 0.36-0.13 (m, 2H).

LCMS (ES, m/z): 462.20 [M+H]⁺.

### Example 3C: Synthesis of trans-6-Chloro-4-((4-(cyclopropyl(phenyl)amino) cyclohexyl)(methyl) amino)-1-methyl-2-oxo-1,2-dihydro-1,5-naphthyridine-3-carbonitrile

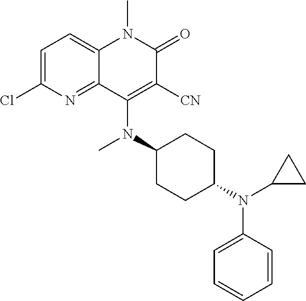 [SMILES: Cn1c(=O)c(C#N)c(N(C)[C@H]2CC[C@H](N(c3ccccc3)C3CC3)CC2)c2nc(Cl)ccc21]

### Step 1: trans-tert-Butyl (4-(cyclopropyl(phenyl)amino)cyclohexyl)(methyl)carbamate

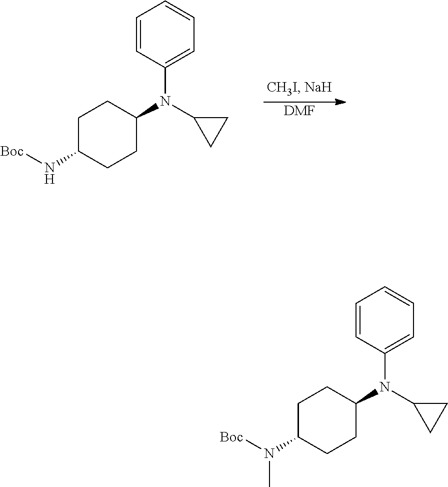

To a stirred solution of trans-tert-butyl (4-(cyclopropyl(phenyl)amino)cyclohexyl) carbamate (440 mg, 1.33 mmol) in DMF (10 mL) was added NaH (96.0 mg, 2.40 mmol, 60% in min. oil), and the mixture was stirred for 0.5 h at rt. Methyl iodide (378 mg, 2.66 mmol) was added and the resulting mixture was stirred for 1 h at rt. It was poured into ice/water (20 mL) and extracted with ethyl acetate (3×25 mL). The combined organic layers were washed with brine (50 mL), dried over anhydrous sodium sulfate and concentrated under reduced pressure. The crude residue was purified by silica gel chromatography with petroleum ether/ethyl acetate (10:1) to afford the title compound (378 mg, 82%) as a light-yellow oil.

LCMS (ES, m/z): 345.30 [M+H]⁺.

### Step 2: trans-(4-(Cyclopropyl(phenyl)amino)cyclohexyl)(methyl)amine hydrotrifluoroacetate

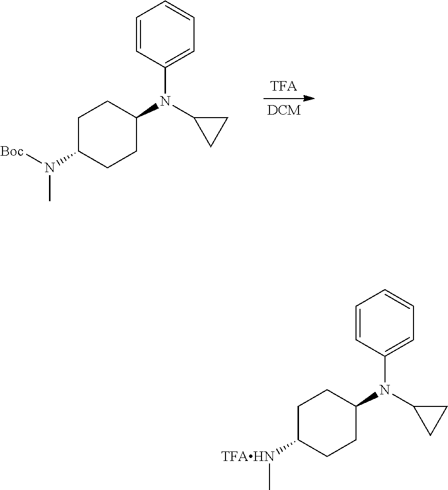

A solution of trans-tert-butyl (4-(cyclopropyl(phenyl)amino)cyclohexyl)(methyl) carbamate (360 mg, 1.05 mmol) in TFA (3.60 mL) and DCM (15 mL) was stirred for 0.5 h at rt. The mixture was concentrated under reduced pressure to afford the title compound (800 mg, assumed quantitative) as a brown oil.

LCMS (ES, m/z): 245.20 [M+H]⁺.

### Step 3: trans-6-Chloro-4-((4-(cyclopropyl(phenyl)amino)cyclohexyl)(methyl)amino)-1-methyl-2-oxo-1,2-dihydro-1,5-naphthyridine-3-carbonitrile

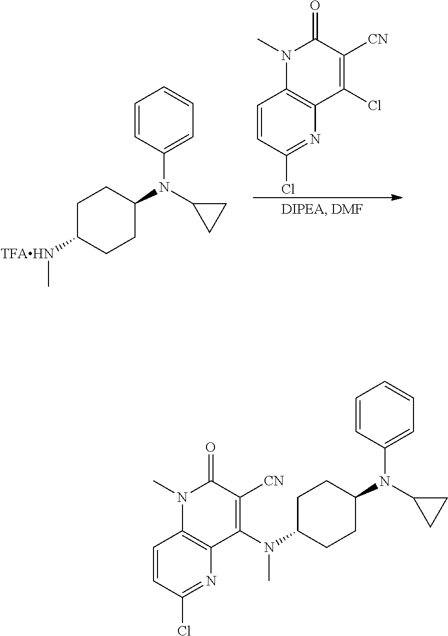

A solution of trans-(4-(cyclopropyl(phenyl)amino)cyclohexyl)(methyl)amine hydrotrifluoro acetate (100 mg, 409 μmol), 4,6-dichloro-1-methyl-2-oxo-1,5-naphthyridine-3-carbonitrile (104 mg, 409 μmol), and DIPEA (528 mg, 4.09 mmol) in DMF (2 mL) was stirred for 2 h at rt. The mixture was poured into water (10 mL) and extracted with ethyl acetate (3×10 mL). The combined organic layers were washed with brine (30 mL), dried over anhydrous sodium sulfate and concentrated under reduced pressure. The crude residue was purified by reversed phase chromatography (Column: Xselect CSH OBD 30×150 mm, 5 um; Mobile Phase, A: Water (10 mmol/L NH₄HCO₃+0.1% NH₃·H₂O) and B: ACN (52% to 72% in 7 min); Detector: 220 nm). The collected fraction was lyophilized to afford the title compound (32.9 mg, 17%) as a yellow solid.

¹H-NMR (DMSO-d₆, 300 MHz) δ (ppm): 8.05 (d, J=9.0 Hz, 1H), 7.80 (d, J=9.0 Hz, 1H), 7.22-7.17 (m, 2H), 7.08-6.98 (m, 2H), 6.75 (t, J=7.2 Hz, 1H), 4.38-4.34 (m, 1H), 3.59-3.52 (m, 4H), 3.24 (s, 3H), 2.34-2.28 (m, 1H), 2.04 (br s, 2H), 2.01-1.73 (m, 6H), 0.85-0.71 (m, 2H), 0.42-0.30 (m, 2H).

LCMS (ES, m/z): 462.25 [M+H]⁺.

### Example 3D: Synthesis of 6-Chloro-4-((trans-4-(cyclopropyl(2-hydroxyphenyl)amino) cyclohexyl)(methyl)amino)-1-methyl-2-oxo-1,2-dihydro-1,5-naphthyridine-3-carbonitrile

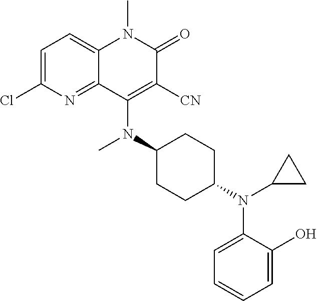 [SMILES: Cn1c(=O)c(C#N)c(N(C)[C@H]2CC[C@H](N(c3ccccc3O)C3CC3)CC2)c2nc(Cl)ccc21]

### Step 1: (2-((2-Bromophenoxy)methoxy)ethyl)trimethylsilane

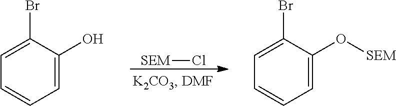 [SMILES: Oc1ccccc1Br ; C[Si](C)(C)CCOCCl ; C[Si](C)(C)CCOCOc1ccccc1Br]

2-bromophenol (7.00 g, 40.5 mmol), SEM-Cl (8.09 g, 48.6 mmol) and potassium carbonate (8.39 g, 60.7 mmol) in DMF (20 mL) were stirred at rt for 1.5 h, then diluted with ice-water (400 mL) and extracted with EtOAc (3×). The combined organic layers were washed with brine, dried over anhydrous sodium sulfate and concentrated under reduced pressure. The crude residue purified by silica gel chromatography (EtOAc-Petroleum ether, 10%), to afford the title compound (11 g, 81%) as a light yellow oil.

GCMS (ES, m/z): 303.27

### Step 2: tert-Butyl (trans-4-((2-((2-(trimethylsilyl)ethoxy)methoxy)phenyl)amino)-cyclohexyl)carbamate

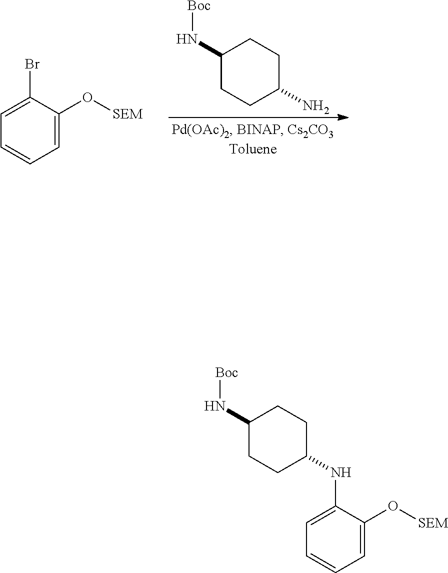 [SMILES: C[Si](C)(C)CCOCOc1ccccc1Br ; CC(C)(C)OC(=O)N[C@H]1CC[C@H](N)CC1 ; CC(C)(C)OC(=O)N[C@H]1CC[C@H](Nc2ccccc2OCOCC[Si](C)(C)C)CC1]

2-[(2-bromophenoxy)methoxy]ethyl-trimethyl-silane (3.60 g, 11.9 mmol), trans-tert-butyl N-(4-aminocyclohexyl)carbamate (1.27 g, 5.94 mmol), diacetoxypalladium (133 mg, 593 μmol), benzyl-[1-[2-[benzyl(phenyl)phosphanyl]-1-naphthyl]-2-naphthyl]-phenyl-phosphane (386 mg, 593 μmol), dicesium carbonate (3.87 g, 11.9 mmol) and toluene (20 mL) were stirred at 110° C. for 12 h under a nitrogen atmosphere. The mixture was cooled to rt, diluted with water and extracted with EtOAc (3×). The combined organic layers were washed with brine, dried over anhydrous sodium sulfate and concentrated under reduced pressure. The residue was purified by silica gel column chromatography (Petroleum ether-EtOAc, 5:1) to afford the title compound (1.3 g, 45%) as a light yellow oil.

LCMS (ES, m/z): 437.47 [M+H]⁺

### Step 3: tert-Butyl (trans-4-(cyclopropyl(2-((2-(trimethylsilyl)ethoxy)methoxy)phenyl) amino)-cyclohexyl)carbamate

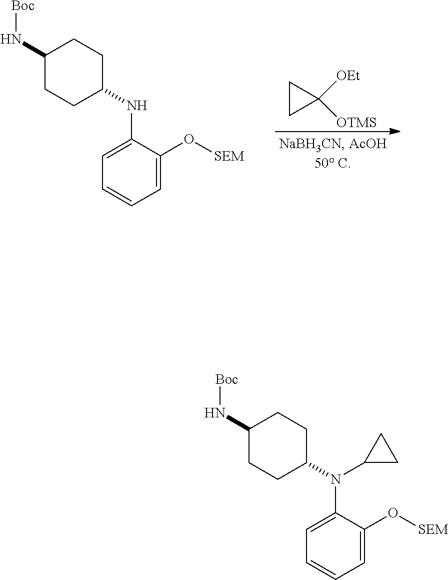

Prepared in a similar manner to Example 24, Step 3

A solution of tert-butyl N-[4-[2-(2-trimethylsilylethoxymethoxy)anilino]cyclohexyl]carbamate (1.30 g, 2.98 mmol), NaBH₃CN (561 mg, 8.93 mmol) and (1-ethoxycyclopropoxy)-trimethyl-silane (2.3 g, 13.2 mmol) in acetic acid (20 mL) was stirred for 1.5 h at 50° C. After cooling to rt, the mixture was diluted with water and extracted with EtOAc (3). The combined organic layers were washed with brine, dried over anhydrous sodium sulfate and concentrated under reduced pressure. The crude residue was purified by silica gel column chromatography (Petroleum ether-EtOAc, 6:1) to afford the title compound (1.3 g, 82%) as a light yellow oil.

LCMS (ES, m/z): 477.43 [M+H]⁺

### Step 4: tert-butyl (trans-4-(cyclopropyl(2-((2-(trimethylsilyl)ethoxy)methoxy)phenyl) amino)-cyclohexyl)(methyl)carbamate

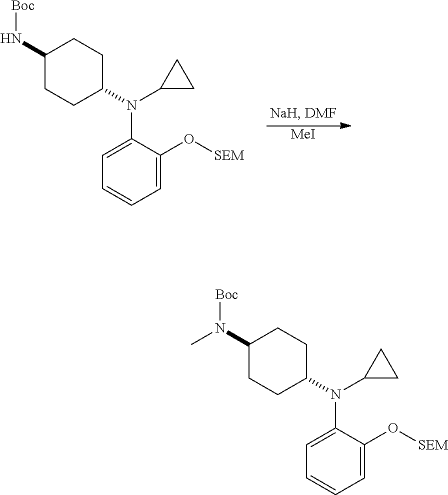 [SMILES: "MeI" ; CC(C)(C)OC(=O)N[C@H]1CC[C@H](N(c2ccccc2OCOCC[Si](C)(C)C)C2CC2)CC1 ; CN(C(=O)OC(C)(C)C)[C@H]1CC[C@H](N(c2ccccc2OCOCC[Si](C)(C)C)C2CC2)CC1]

To a solution of tert-butyl (trans-4-(cyclopropyl(2-((2-(trimethylsilyl)ethoxy)methoxy) phenyl) amino)cyclohexyl) carbamate (200 mg, 419 μmol) in DMF (8 mL) at 0 C was added NaH (50 mg, 1.26 mmol, 60% in min. oil). After 30 mins, iodomethane (178 mg, 1.26 mmol) was added dropwise to the mixture, which was then stirred at 0° C. for 1.5 h, then quenched with saturated NH₄Cl (aq) and extracted with EtOAc (3×). The combined organic layers were washed with brine, dried over anhydrous sodium sulfate and concentrated under reduced pressure. The crude residue was purified by silica gel column chromatography (Petroleum ether-EtOAc, 8:1) to afford the title compound (200 mg, 86%) as light yellow oil.

LCMS (ES, m/z): 491.22 [M+H]⁺

### Step 5: 2-(Cyclopropyl(trans-4-(methylammonium)cyclohexyl)amino)phenol trifluoroacetate

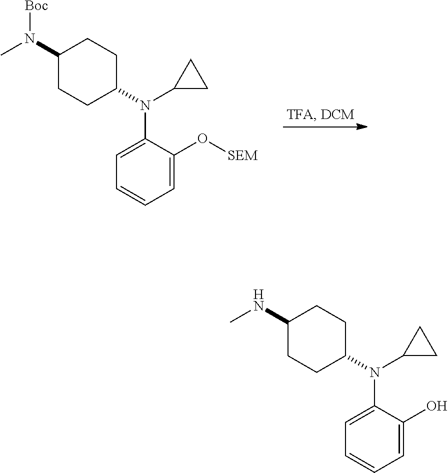 [SMILES: CN(C(=O)OC(C)(C)C)[C@H]1CC[C@H](N(c2ccccc2OCOCC[Si](C)(C)C)C2CC2)CC1 ; CN[C@H]1CC[C@H](N(c2ccccc2O)C2CC2)CC1]

To a solution of tert-butyl (trans-4-(cyclopropyl(2-((2-(trimethylsilyl)ethoxy)methoxy) phenyl)amino)cyclohexyl)(methyl)carbamate (200 mg, 407 μmol) in DCM (8 mL) was added 2,2,2-trifluoroacetic acid (1 mL). After 2 h at rt, the mixture was concentrated under reduced pressure, basified to pH 8 with saturated NaHCO₃(aq) at 0° C. and extracted with EtOAc (3×). The combined organic layers were washed with brine, dried over anhydrous sodium sulfate and concentrated under reduced pressure to afford the title compound (189 mg, 93%) as a brown oil.

LCMS (ES, m/z): 261.22 [M-TFA+H]+

### Step 6: 6-Chloro-4-((trans-4-(cyclopropyl(2-hydroxyphenyl)amino)cyclohexyl)(methyl) amino)-1-methyl-2-oxo-1,2-dihydro-1,5-naphthyridine-3-carbonitrile

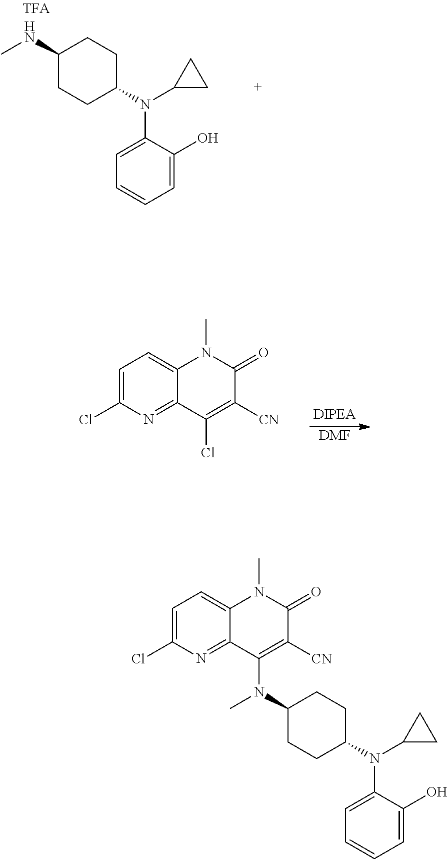

A solution of 2-(cyclopropyl(trans-4-(methylammonium)cyclohexyl)amino)phenol trifluoroacetate (138 mg, 275 μmol), 4,6-dichloro-1-methyl-2-oxo-1,2-dihydro-1,5-naphthyridine-3-carbonitrile (70 mg, 275 μmol) and DIPEA (178 mg, 1.38 mmol) in DMF (5 mL) was stirred at rt for 1 h. The mixture was poured into water (10 mL) and extracted with EtOAc (3×10 mL). The combined organic layers were washed with brine (30 mL), dried over anhydrous sodium sulfate and concentrated under reduced pressure. The residue was purified by Prep-HPLC (Column: Xselect CSH OBD Column 30×150 mm, 5 um; Mobile Phase, A: water (10 mmol/L NH₄HCO₃+0.1% NH₃·H₂O) and B: ACN (52% to 72% in 7 min); Detector: 220 nm) to afford the title compound (38.6 mg, 28%) as a light yellow solid.

¹H NMR (300 MHz, DMSO-d₆) δ 8.26 (s, 1H), 8.04 (d, J=9.1 Hz, 1H), 7.78 (d, J=8.9 Hz, 1H), 7.18-7.13 (m, 1H), 6.94-6.87 (m, 1H), 6.78-6.72 (m, 2H), 4.29-4.16 (m, 1H), 3.51 (s, 3H), 3.19 (s, 3H), 3.16-3.05 (m, 1H), 2.07-1.93 (m, 4H), 1.78 (q, J=11.7 Hz, 2H), 1.61-1.45 (m, 2H), 0.52-0.45 (m, 2H), 0.31 (br s, 2H).

LCMS (ES, m/z): 478.25, 480.25 [M+H]⁺

### Example 3E: Synthesis of trans-6-chloro-4-((4-(cyclopropyl(4-methoxyphenyl) amino) cyclohexyl)(methyl)amino)-1-methyl-2-oxo-1,2-dihydro-1,5-naphthyridine-3-carbonitrile

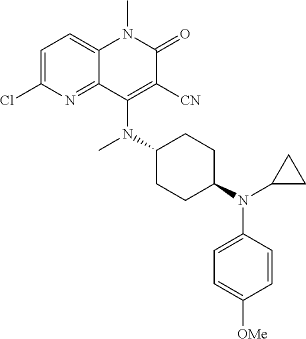 [SMILES: COc1ccc(N(C2CC2)[C@H]2CC[C@H](N(C)c3c(C#N)c(=O)n(C)c4ccc(Cl)nc34)CC2)cc1]

### Step 1: trans-tert-Butyl N-[4-(4-methoxyanilino)cyclohexyl]carbamate

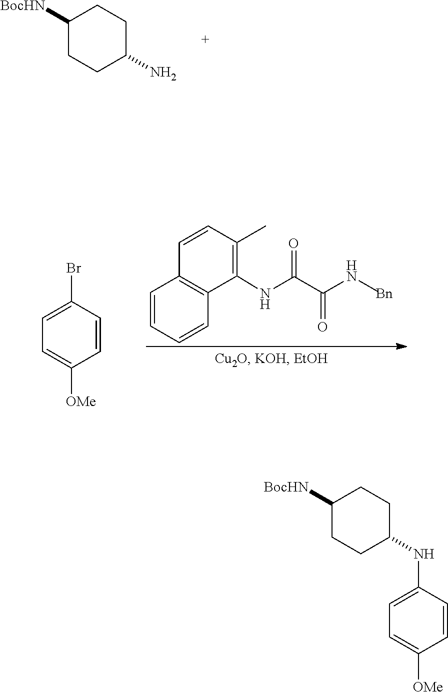

A mixture of trans-tert-butyl N-(4-aminocyclohexyl)carbamate (1.7 g, 7.93 mmol), 1-bromo-4-methoxy-benzene (1.0 g, 5.35 mmol), Cu₂O (76 mg, 531.13 μmol), KOH (390 mg, 6.95 mmol) and N¹-(2-methyl-1-naphthalenyl)-N²-(phenylmethyl)-ethanediamide (170 mg, 535 μmol) in ethanol (20 mL) heated to 80° C. for 16 h, whereupon it was concentrated, diluted with water (50 mL) and extracted with DCM (2×50 mL). The combined organic layers were washed with brine (50 mL), dried over anhydrous sodium sulfate and concentrated under reduced pressure. The crude residue was purified via silica gel chromatography (petroleum ether-EtOAc, 1:1) to afford the title compound (1.1 g, 3.1 mmol, 58%) as white solid.

LCMS (ES, m/z): 321.20 [M+H]⁺

### Steps 2-5: trans-6-Chloro-4-[[4-(N-cyclopropyl-4-methoxy-anilino)cyclohexyl]-methyl-aminol-1-methyl-2-oxo-1,5-naphthyridine-3-carbonitrile

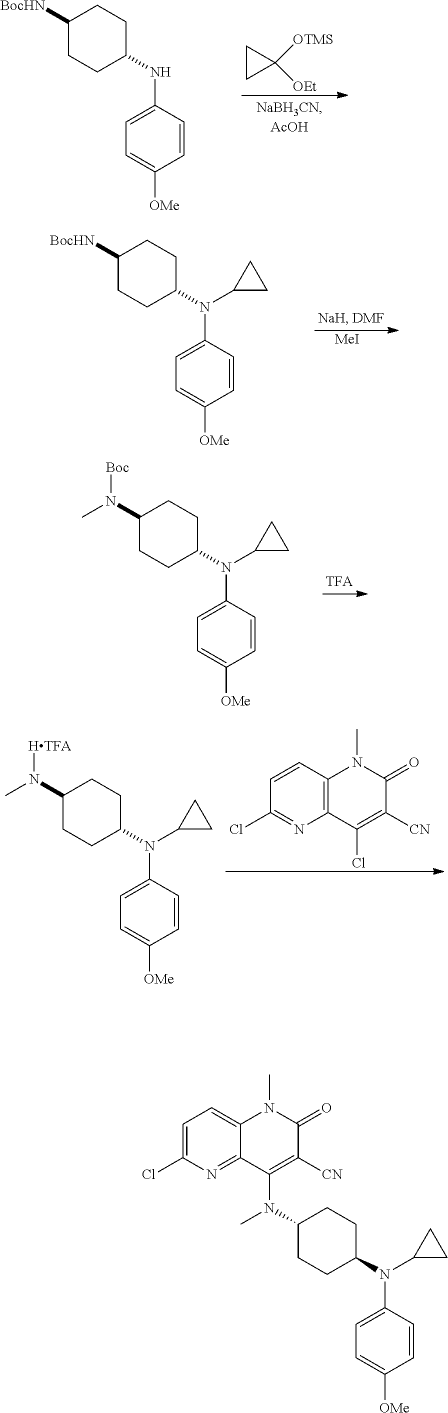

Carried out in an analogous manner to examples as described herein.

The mixture was purified by reverse flash chromatography (Column, C18 silica gel, 80 g, 20-35 um; Mobile phase, A: water with 10 mmol/L NH₄HCO₃ and B: ACN, 0-100% over 30 min; Detector: UV 254 nm), affording the title compound (33.8 mg, 62.7 μmol, 13%) as yellow solid.

¹H NMR (400 MHz, DMSO-d₆) δ 8.04 (d, J=9.0 Hz, 1H), 7.80 (d, J=8.9 Hz, 1H), 6.99 (d, J=8.6 Hz, 2H), 6.80 (d, J=8.8 Hz, 2H), 4.32-4.23 (m, 1H), 3.69 (s, 3H), 3.52 (s, 3H), 3.22 (s, 3H), 2.34 (br s, 1H), 2.09-2.00 (m, 2H), 1.93-1.82 (m, 4H), 1.72-1.61 (m, 2H), 0.71-0.65 (m, 2H), 0.31-0.26 (m, 2H).

LCMS (ES, m/z): 492.25 [M+H]⁺.

### Example 3F: Synthesis of trans-6-chloro-4-((4-(cyclopropyl(4-fluorophenyl)amino) cyclohexyl) (methyl)amino)-1-methyl-2-oxo-1,2-dihydro-1,5-naphthyridine-3-carbonitrile

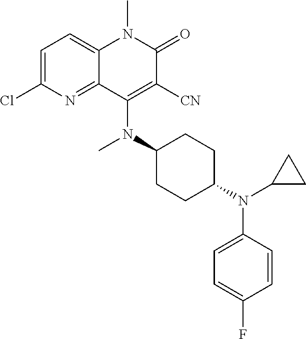 [SMILES: Cn1c(=O)c(C#N)c(N(C)[C@H]2CC[C@H](N(c3ccc(F)cc3)C3CC3)CC2)c2nc(Cl)ccc21]

The title compound was prepared in a similar manner to Example 3D.

The final product was purified by SFC (Column: GreenSep Naphthyl, 3×25 cm, 5 m; Mobile Phase, A: CO₂ and B: ACN:MeOH, 4:1 with 0.1% 2M NH₃-MeOH, hold 42% over 8 min; Detector: 254 nm), affording the title compound (14.2 mg, 28.9 μmol, 7.8%) as a yellow solid.

¹H NMR (400 MHz, DMSO-d₆) δ 8.05 (d, J=9.0 Hz, 1H), 7.80 (d, J=9.0 Hz, 1H), 7.03 (d, J=6.7 Hz, 4H), 4.37-4.28 (m, 1H), 3.52 (s, 3H), 3.50-3.41 (m, 1H), 3.22 (s, 3H), 2.35-2.29 (m, 1H), 2.08-2.00 (m, 2H), 1.97-1.85 (m, 4H), 1.82-1.70 (m, 2H), 0.77-0.72 (m, 2H), 0.35-0.29 (m, 2H).

LCMS (ES, m/z): 480.15 [M+H]⁺

### Example 3G: Synthesis of trans-6-chloro-4-((4-(cyclopropyl(4-fluoro-2-hydroxyphenyl)amino) cyclohexyl)(methyl)amino)-1-methyl-2-oxo-1,2-dihydro-1,5-naphthyridine-3-carbonitrile

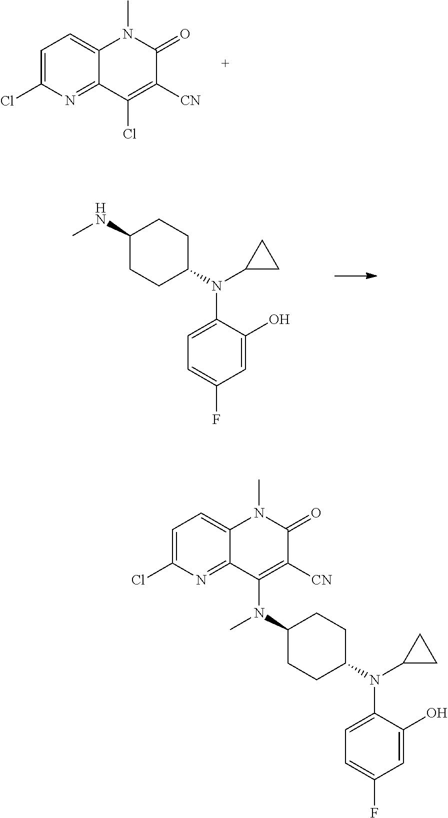 [SMILES: Cn1c(=O)c(C#N)c(Cl)c2nc(Cl)ccc21 ; CN[C@H]1CC[C@H](N(c2ccc(F)cc2O)C2CC2)CC1 ; Cn1c(=O)c(C#N)c(N(C)[C@H]2CC[C@H](N(c3ccc(F)cc3O)C3CC3)CC2)c2nc(Cl)ccc21]

A solution of trans-2-[cyclopropyl-[4-(methylamino)cyclohexyl]amino]-5-fluoro-phenol (200 mg, 718 μmol), 4,6-dichloro-1-methyl-2-oxo-1,5-naphthyridine-3-carbonitrile (182 mg, 718.48 μmol) and DIEA (438 mg, 2.16 mmol) in DMF (3 mL) was stirred at rt for 1 h. The mixture was purified by HPLC (Column: XBridge BEH C18 OBD Prep Column, 5 μm, 19×250 mm; Mobile Phase, A: water (10 mmol/L NH₄HCO₃+0.1% NH₃·H₂O) and B: ACN (60-80% in 9 min); Detector: 254 nm), affording the title compound (50.9 mg, 103 μmol, 14%) as a light-yellow solid.

¹H NMR (400 MHz, Methanol-d₄) δ 7.88 (d, J=9.0 Hz, 1H), 7.56 (d, J=8.9 Hz, 1H), 7.17-7.11 (m, 1H), 6.48-6.42 (m, 2H), 4.37-4.28 (m, 1H), 3.51 (s, 3H), 3.23 (s, 3H), 2.99-2.91 (m, 1H), 2.48-2.41 (m, 1H), 2.04 (br d, J=11.2 Hz, 4H), 1.83-1.72 (m, 2H), 1.48-1.37 (m, 2H), 0.45-0.38 (m, 2H), 0.35-0.27 (m, 2H).

LCMS (ES, m/z): 496.20 [M+H]⁺

### Example 3H: Synthesis of cis-6-chloro-4-((4-(cyclopropyl(4-fluoro-2-methoxyphenyl)amino) cyclohexyl)(methyl)amino)-1-methyl-2-oxo-1,2-dihydro-1,5-naphthyridine-3-carbonitrile

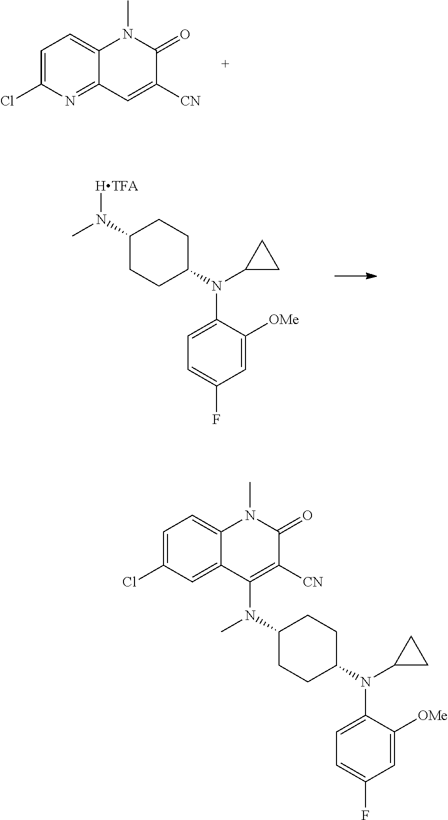

To a solution of N⁴-cyclopropyl-N⁴-(4-fluoro-2-methoxy-phenyl)-N¹-methyl-cyclohexane-1,4-diamine hydrotrifluoroacetate (300 mg, 738 μmol), 4,6-dichloro-1-methyl-2-oxo-1,5-naphthyridine-3-carbonitrile (188 mg, 738 μmol) in DMF (3 mL) was added DIEA (751 mg, 3.69 mmol). After 1 h, the solution was directly purified by SFC (Column: Viridis BEH 2-Ethylpyridine Prep OBD, 3×15 cm, 5 μm; Mobile Phase, A: C₀₂ and B: 20% MeOH with 0.5% 2 M NH₃-MeOH, over 10 min), affording the title compound (46.5 mg, 83.6 μmol, 11%) as a yellow solid.

¹H NMR (400 MHz, DMSO-d₆) δ 8.03 (d, J=9.0 Hz, 1H), 7.78 (d, J=9.0 Hz, 1H), 7.16-7.09 (m, 1H), 6.89 (dd, J=11.1, 2.8 Hz, 1H), 6.68 (td, J=8.7, 4.3 Hz, 1H), 4.36-4.28 (m, 1H), 3.79 (s, 3H), 3.51 (s, 3H), 3.47 (s, 1H), 3.25 (s, 3H), 2.42 (br s, 1H), 2.14-2.01 (m, 2H), 2.00-1.89 (m, 2H), 1.61 (bd, J=11.1 Hz, 2H), 1.43 (t, J=13.4 Hz, 2H), 0.45-0.35 (m, 4H).

LCMS (ES, m/z): 510.25 [M+H]⁺

### Example 31: Synthesis of trans-6-Chloro-4-[[4-[cyclopropyl-(5-fluoro-2-pyridyl)aminol cyclohexyl]-methyl-amino]-1-methyl-2-oxo-1,5-naphthyridine-3-carbonitrile

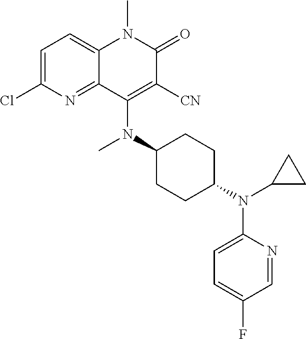 [SMILES: Cn1c(=O)c(C#N)c(N(C)[C@H]2CC[C@H](N(c3ccc(F)cn3)C3CC3)CC2)c2nc(Cl)ccc21]

Prepared in a similar manner to examples as described herein, according to the scheme below

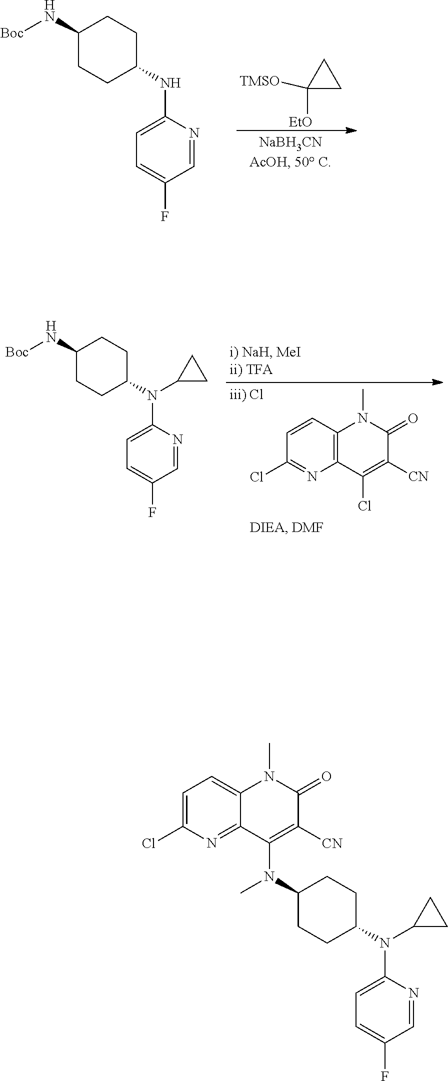

The final product was purified by reverse flash chromatography (Column, C18 silica gel, 80 g, 20-35 um; Mobile phase, A: water (0.5% TFA) and B: ACN (30% to 85% in 30 min); Detector, UV 254 nm) to afford the title compound (61.2 mg, 116 μmol, 15%) as an off-white solid.

¹H NMR (300 MHz, DMSO-d₆) δ 8.12 (d, J=3.1 Hz, 1H), 8.06 (d, J=9.0 Hz, 1H), 7.81 (d, J=9.0 Hz, 1H), 7.51 (td, J=8.8, 3.1 Hz, 1H), 7.00 (dd, J=9.3, 3.6 Hz, 1H), 4.39 (br s, 1H), 4.26 (br s, 1H), 3.53 (s, 3H), 3.25 (s, 3H), 2.41-2.34 (m, 1H), 2.12-2.00 (m, 2H), 2.00-1.82 (m, 6H), 0.92-0.85 (m, 2H), 0.56-0.49 (m, 2H).

LCMS (ES, m/z): 481.15, 483.15 [M+H]⁺.

### Example 3J: Synthesis of trans-6-chloro-4-((4-(cyclopropyl(5-methoxypyridin-2-yl) amino) cyclohexyl)(methyl)amino)-1-methyl-2-oxo-1,2-dihydro-1,5-naphthyridine-3-carbonitrile

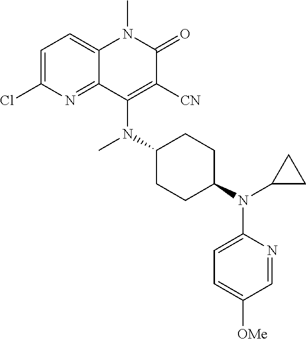 [SMILES: COc1ccc(N(C2CC2)[C@H]2CC[C@H](N(C)c3c(C#N)c(=O)n(C)c4ccc(Cl)nc34)CC2)nc1]

The title compound was prepared in a similar manner to Example 3D

The final product was purified by reverse flash chromatography (Column: C18 silica gel, 80 g, 20-35 um; Mobile Phase, A: water (10 mmol/L NH₄HCO₃) and B: ACN (5% to 80% in 50 min); Detector: 254 nm), affording the title compound (53.4 mg, 108 μmol, 19%) as a light-yellow solid.

¹H NMR (400 MHz, DMSO-d₆) δ 8.06 (d, J=9.0 Hz, 1H), 7.91 (d, J=3.1 Hz, 1H), 7.81 (d, J=8.8 Hz, 1H), 7.26 (dd, J=9.2, 3.2 Hz, 1H), 6.95 (d, J=9.0 Hz, 1H), 4.42-4.32 (m, 1H), 4.29-4.20 (m, 1H), 3.73 (s, 3H), 3.52 (s, 3H), 3.25 (s, 3H), 2.34-2.28 (m, 1H), 2.12-2.03 (m, 2H), 1.99-1.76 (m, 6H), 0.88-0.81 (m, 2H), 0.52-0.44 (m, 2H).

LCMS (ES, m/z): 493.15 [M+H]⁺

### Example 3K: Synthesis of cis-6-Chloro-4-((4-((cyclopropylmethyl)(4-fluorophenyl) amino) cyclohexyl)amino)-1-methyl-2-oxo-1,2-dihydro-1,5-naphthyridine-3-carbonitrile

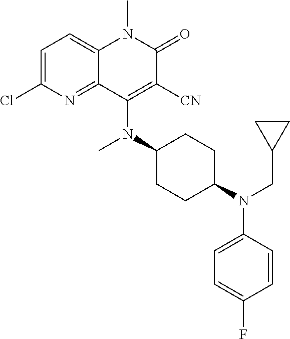 [SMILES: Cn1c(=O)c(C#N)c(N(C)[C@H]2CC[C@@H](N(CC3CC3)c3ccc(F)cc3)CC2)c2nc(Cl)ccc21]

### Step 1: cis-Benzyl (4-((cyclopropylmethyl)(4-fluorophenyl)amino)cyclohexyl)carbamate

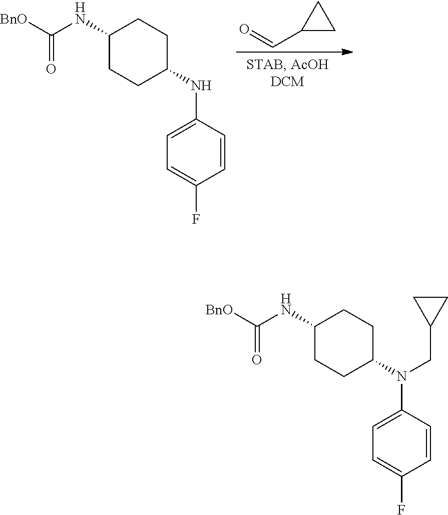 [SMILES: O=C(N[C@H]1CC[C@@H](Nc2ccc(F)cc2)CC1)OCc1ccccc1 ; O=CC1CC1 ; O=C(N[C@H]1CC[C@@H](N(CC2CC2)c2ccc(F)cc2)CC1)OCc1ccccc1]

The compound was prepared according to similar methods as described herein, using cis-benzyl (4-((4-fluorophenyl)amino)cyclohexyl)carbamate (250 mg, 730 μmol), cyclopropanecarbaldehyde (768 mg, 11.0 mmol, 819 μL), DCM (3.4 mL), acetic acid (11.0 mg, 183 μmol, 10.5 μL), and sodium triacetoxyborohydride (1.91 g, 8.76 mmol), to afford the title compound (247 mg, 85%) as an oil.

LCMS (ES, m/z): 397.51 [M+H]⁺.

### Step 2: cis-N-(Cyclopropylmethyl)-N-(4-fluorophenyl)cyclohexane-1,4-diamine

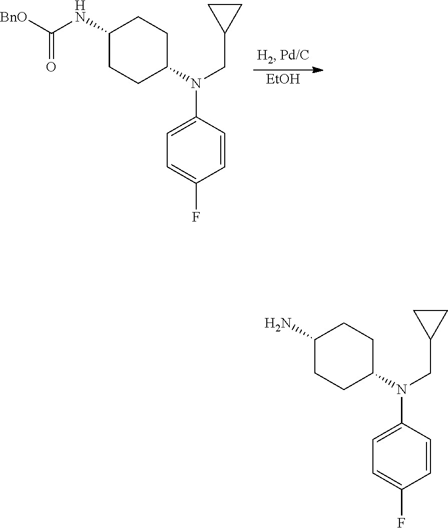 [SMILES: "EtOH" ; O=C(N[C@H]1CC[C@@H](N(CC2CC2)c2ccc(F)cc2)CC1)OCc1ccccc1 ; N[C@H]1CC[C@@H](N(CC2CC2)c2ccc(F)cc2)CC1]

The compound was prepared according to similar methods as described herein, using cis-benzyl (4-((cyclopropylmethyl)(4-fluorophenyl)amino)cyclohexyl)carbamate (245 mg, 618 μmol), palladium (10% on carbon, 65.8 mg), ethanol (3.1 mL), and hydrogen, to afford the title compound (145 mg, 89%) as an oil.

LCMS (ES, m/z): 263.43 [M+H]⁺.

### Step 3: cis-8-((4-((Cyclopropylmethyl)(4-fluorophenyl)amino)cyclohexyl)amino)-5-methyl-6-oxo-5,6-dihydro-1,5-naphthyridine-2-carbonitrile

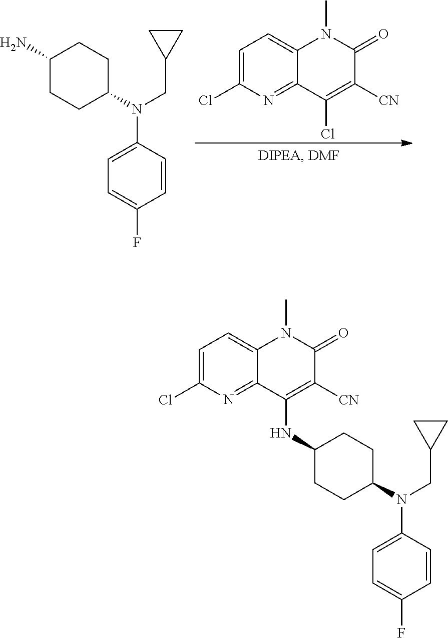

The compound was prepared according to similar methods as described herein, using cis-N-(cyclopropylmethyl)-N-(4-fluorophenyl)cyclohexane-1,4-diamine (58.1 mg, 222 μmol), DMA (953 μL), DIPEA (115 mg, 886 μmol, 154 μL) and 8-chloro-5-methyl-6-oxo-5,6-dihydro-1,5-naphthyridine-2-carbonitrile (61.9 mg, 244 μmol), and purified by silica gel chromatography with ethyl acetate/heptane (0-100%) to afford the title compound (145 mg, 89%) as an oil.

LCMS (ES, m/z): 482.46 [M+H]⁺.

### Step 4: cis-6-Chloro-4-((4-((cyclopropylmethyl)(4-fluorophenyl)amino)cyclohexyl) amino)-1-methyl-2-oxo-1,2-dihydro-1,5-naphthyridine-3-carbonitrile

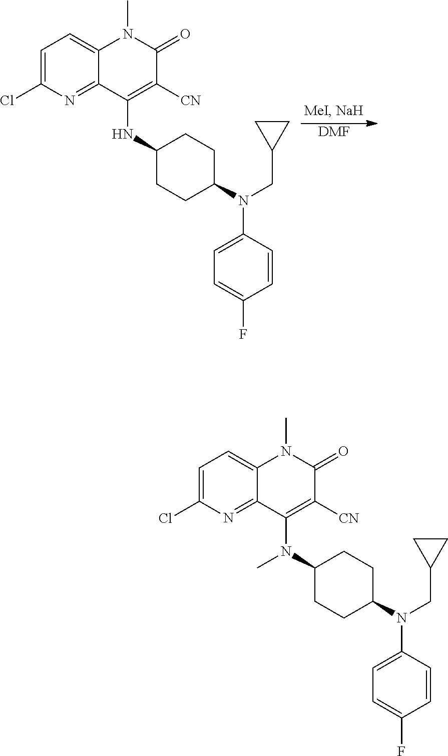

To a solution of cis-8-((4-((cyclopropylmethyl)(4-fluorophenyl)amino)cyclohexyl) amino)-5-methyl-6-oxo-5,6-dihydro-1,5-naphthyridine-2-carbonitrile (59.2 mg, 123 μmol) in DMF (2.4 mL) was added NaH (3.00 mg, 130 μmol, 60% in min. oil), and the mixture was stirred for 0.3 h at rt. Methyl iodide (19.1 mg, 135 μmol) was added, and the resulting mixture was stirred for 1 h at rt. The mixture was quenched with HCl (1N, 184 uL), filtered and concentrated under reduced pressure. The residue was purified by reversed phase chromatography (Column: SunFire Prep OBD C18, 19×250 mm, 5 μm; Mobile Phase, A: water (0.05% formic acid) and B: ACN (0.05% formic acid, 10-50% in 10 min); Detector: UV254/220 nm). The collected fractions were lyophilized to afford the title compound (26.9 mg, 45%) as a white solid.

¹H-NMR (DMSO-d₆, 300 MHz) δ (ppm): 8.09 (d, J=9.1 Hz, 1H), 7.82 (d, J=8.9 Hz, 1H), 7.26-7.18 (m, 2H), 7.17-7.05 (m, 2H), 4.47-4.30 (m, 1H), 3.60-3.50 (s, 3H), 3.28 (s, 3H), 2.92 (d, J=6.1 Hz, 2H), 2.22-2.04 (m, 2H), 1.87 (br d, J=11.7 Hz, 2H), 1.70 (br d, J=8.8 Hz, 2H), 1.49 (br t, J=11.6 Hz, 2H), 0.72 (br s, 1H), 0.34 (d, J=7.4 Hz, 2H), −0.04 (d, J=4.3 Hz, 2H).

LCMS (ES, m/z): 494.56 [M+H]⁺.

### Example 3L: Synthesis of trans-6-chloro-4-((4-((cyclopropylmethyl)(5-fluoropyridin-2-yl)amino) cyclohexyl)(methyl)amino)-1-methyl-2-oxo-1,2-dihydro-1,5-naphthyridine-3-carbonitrile

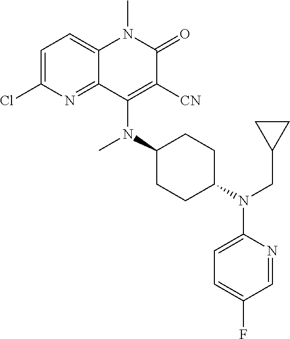 [SMILES: Cn1c(=O)c(C#N)c(N(C)[C@H]2CC[C@H](N(CC3CC3)c3ccc(F)cn3)CC2)c2nc(Cl)ccc21]

### Step 1: trans-tert-butyl N-[4-[cyclopropylmethyl-(5-fluoro-2-pyridyl)aminol cyclohexyl]-carbamate

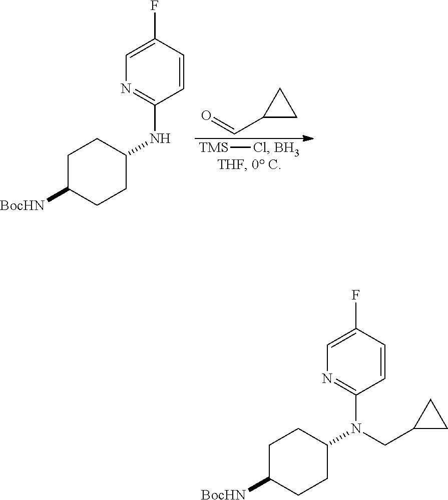

To a mixture of trans-tert-butyl N-[4-[(5-fluoro-2-pyridyl)amino]cyclohexyl]carbamate (1.4 g, 4.53 mmol), cyclopropanecarbaldehyde (349 mg, 4.98 mmol) and TMS-Cl (1.23 g, 11.31 mmol) in THF (20 mL) at 0° C. was added BH₃ (1 M in THF, 4.53 mL). After stirring for 3 h at 0° C., the solution was poured into water (15 mL) and stirred for 5 min. Saturated Na₂CO₃ (aq, 20 mL) was added and the mixture was stirred until the gas evolution had ceased. The mixture was extracted with EtOAc (3×30 mL) and the combined organic layers were washed with brine (30 mL), dried over anhydrous sodium sulfate and concentrated under reduced pressure. The residue was purified by silica gel column chromatography (Petroleum ether-EtOAc, 2:1) to afford the title compound (800 mg, 2.09 mmol, 46%) as a white solid.

LCMS (ES, m/z): 364.20 [M+H]⁺.

### Step 2: trans-tert-Butyl N-[4-[cyclopropylmethyl-(5-fluoro-2-pyridyl)aminol cyclohexyl]-N-methyl-carbamate

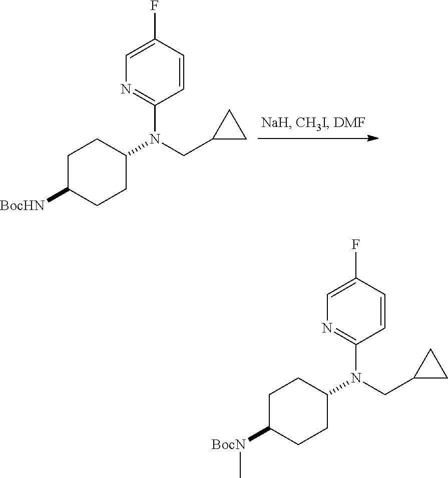

To a mixture of trans-tert-butyl N-[4-[cyclopropylmethyl-(5-fluoro-2-pyridyl)amino]cyclohexyl]carbamate (830 mg, 2.28 mmol) in DMF (5 mL) at 0° C. was added NaH (274 mg, 6.85 mmol, 60% in min. oil) portion-wise. After 30 min at 0° C., Mel (1.62 g, 11.42 mmol) was added. After an additional 1 h at 0° C., the mixture was quenched with saturated NH₄Cl (aq, 20 mL) and extracted with EtOAc (3×30 mL). The combined organic layers were washed with brine (30 mL), dried over anhydrous sodium sulfate and concentrated under reduced pressure. The crude residue was purified by silica gel chromatography (Petroleum ether-EtOAc, 1:1) to afford the title compound (420 mg, 1.00 mmol, 44%) as a yellow oil.

LCMS (ES, m/z): 378.20 [M+H]⁺.

### Step 3: trans-N¹-(Cyclopropylmethyl)-N¹-(5-fluoropyridin-2-yl)-N₄-methylcyclohexane-1,4-diamine hydrotrifluoroacetate

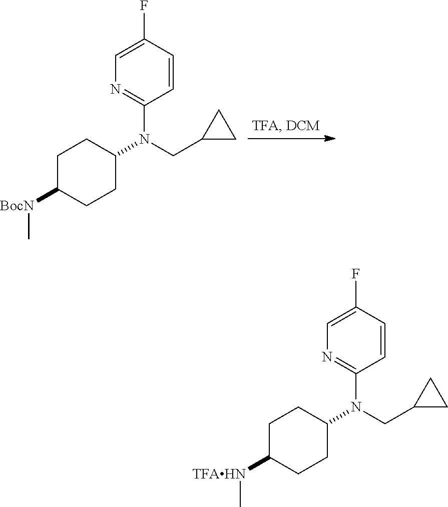

A mixture of trans-tert-butyl N-[4-[cyclopropylmethyl-(5-fluoro-2-pyridyl)amino]cyclohexyl]-N-methyl-carbamate (420 mg, 1.11 mmol) in DCM (3 mL) and TFA (1 mL) was stirred for 1 h. The solution was concentrated to afford the title compound (320 mg, 923 μmol, 59%, 80% purity) as a brown oil.

LCMS (ES, m/z): 278.50 [M-TFA+H]⁺

### Step 4: trans-6-Chloro-4-[[4-[cyclopropylmethyl-(5-fluoro-2-pyridyl)aminol cyclohexyl]-methyl-amino]-1-methyl-2-oxo-1,5-naphthyridine-3-carbonitrile

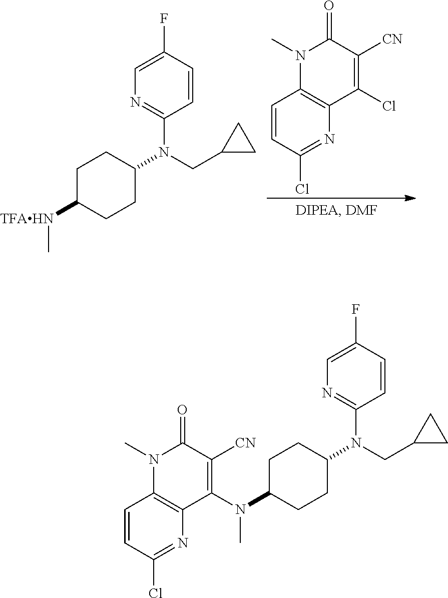

A mixture of trans-N¹-(cyclopropylmethyl)-N¹-(5-fluoropyridin-2-yl)-N⁴-methylcyclohexane-1,4-diamine hydrotrifluoroacetate (150 mg, 306.6 μmol, 80%), 4,6-dichloro-1-methyl-2-oxo-1,5-naphthyridine-3-carbonitrile (77.9 mg, 306.6 μmol) and DIPEA (118.9 mg, 919.8 μmol) in DMF (3 mL) was stirred for 2 h. The crude mixture was directly purified by reverse flash chromatography (Column, C18 silica gel, 80 g, 20-35 um; Mobile phase, A: water (0.1% TFA) and B: ACN (30% to 80% in 20 min); Detector: UV 254 nm), affording the title compound (39.1 mg, 74.3 μmol, 24%) as a yellow solid.

¹H NMR (300 MHz, DMSO-d₆) δ 8.08-8.03 (m, 2H), 7.81 (d, J=8.9 Hz, 1H), 7.47 (dt, J=8.8, 5.3 Hz, 1H), 6.74 (dd, J=9.3, 3.3 Hz, 1H), 4.44-4.21 (m, 2H), 3.52 (s, 3H), 3.26 (s, 3H), 3.19 (d, J=6.0 Hz, 2H), 2.13-1.92 (m, 4H), 1.85-1.60 (m, 4H), 1.01-0.89 (m, 1H), 0.50-0.42 (m, 2H), 0.31-0.23 (m, 2H).

LCMS (ES, m/z): 495.15 [M+H]⁺.

### Example 3M: Synthesis of cis-6-Chloro-4-[[4-(N-4-fluorophenyl-N-oxetan-3-ylmethylanilino) cyclohexyl]-methylamino]-1-methyl-2-oxo-1,2-dihydro-1,5-naphthyridine-3-carbonitrile

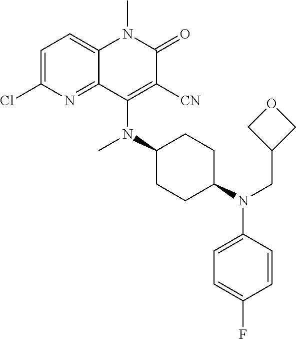 [SMILES: Cn1c(=O)c(C#N)c(N(C)[C@H]2CC[C@@H](N(CC3COC3)c3ccc(F)cc3)CC2)c2nc(Cl)ccc21]

Prepared in a similar manner to examples as described herein. The crude material was purified by HPLC (Column: Sunfire OBD, C18, 19×250 mm, 5 μm; Mobile Phase, A: water with 0.1% formic acid and B: ACN with 0.1% formic acid, 25-50% over 10 min), affording the title compound (8.2 mg, 35%) as white solid.

¹H NMR (300 MHz, DMSO-d₆) δ ppm 8.06 (d, J=9.2 Hz, 1H), 7.79 (d, J=8.6 Hz, 1H), 7.25-7.08 (m, 4H), 4.47-4.29 (m, 3H), 4.10 (t, J=6.1 Hz, 2H), 3.53 (s, 3H), 3.29-3.18 (m, 6H), 2.91-2.78 (m, 1H), 2.11 (q, J=11.5 Hz, 2H), 1.84 (br d, J=13.2 Hz, 2H), 1.66 (br d, J=10.0 Hz, 2H), 1.53-1.35 (t, J=12.6 Hz, 2H).

LCMS (ES, m/z): 510.53 [M+H]⁺.

### Example 3N: Synthesis of trans-6-Chloro-4-[[4-(N-4-fluorophenyl-N-oxetan-3-ylmethylanilino) cyclohexyl]-methylamino]-1-methyl-2-oxo-1,2-dihydro-1,5-naphthyridine-3-carbonitrile

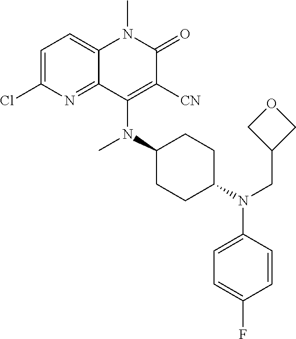 [SMILES: Cn1c(=O)c(C#N)c(N(C)[C@H]2CC[C@H](N(CC3COC3)c3ccc(F)cc3)CC2)c2nc(Cl)ccc21]

Prepared in a similar manner to examples as described herein, employing the methylation described in example 3K for the final step. The crude material was purified by HPLC (Column: Sunfire OBD, C18, 19×250 mm, 5 μm; Mobile Phase, A: water with 0.1% formic acid and B: ACN with 0.1% formic acid, 25-50% over 10 min), affording the title compound (16.9 mg, 39%) as white solid.

LCMS (ES, m/z): 510.53 [M+H]⁺.

### Example 30: Synthesis of trans-6-chloro-4-((4-((4-fluoro-2-hydroxyphenyl)(oxetan-3-ylmethyl) amino)cyclohexyl)(methyl)amino)-1-methyl-2-oxo-1,2-dihydro-1,5-naphthyridine-3-carbonitrile

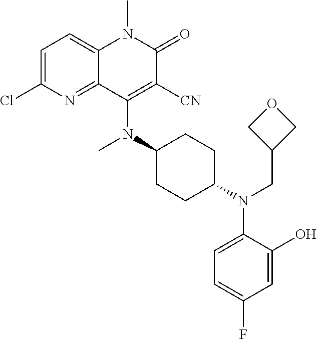 [SMILES: Cn1c(=O)c(C#N)c(N(C)[C@H]2CC[C@H](N(CC3COC3)c3ccc(F)cc3O)CC2)c2nc(Cl)ccc21]

### Step 1: trans-6-Chloro-4-[[4-[4-fluoro-N-(oxetan-3-ylmethyl)-2-(2-trimethylsilylethoxy methoxy)anilino]cyclohexyl]-methyl-aminol-1-methyl-2-oxo-1,5-naphthyridine-3-carbonitrile

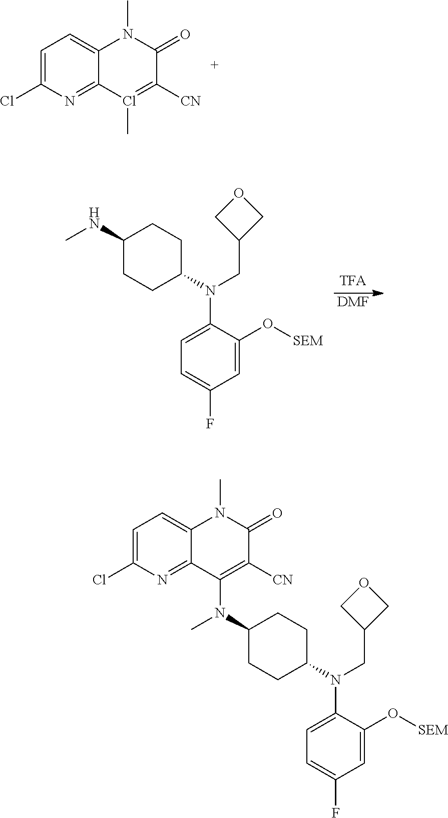

To a solution of trans-N⁴-[4-fluoro-2-(2-trimethylsilylethoxymethoxy)phenyl]-N¹-methyl-N⁴-(oxetan-3-ylmethyl)cyclohexane-1,4-diamine (300 mg, 684 μmol), 4,6-dichloro-1-methyl-2-oxo-1,5-naphthyridine-3-carbonitrile (174 mg, 684 μmol) in DMF (5 mL) was added TEA (208 mg, 2.05 mmol). After 1 h, it was directly purified by reverse flash chromatography (Column: C18 silica gel, 80 g, 20-35 um; Mobile Phase, A: water with 10 mmol/L NH₄HCO₃ and B: ACN, 5-75% over 50 min), affording the title compound (250 mg, 381 μmol, 56%) as a yellow oil.

LCMS (ES, m/z): 656.45 [M+H]⁺.

### Step 2: trans-6-Chloro-4-[[4-[4-fluoro-2-hydroxy-N-(oxetan-3-ylmethyl)anilino]cyclohexyl]-methyl-amino]-1-methyl-2-oxo-1,5-naphthyridine-3-carbonitrile

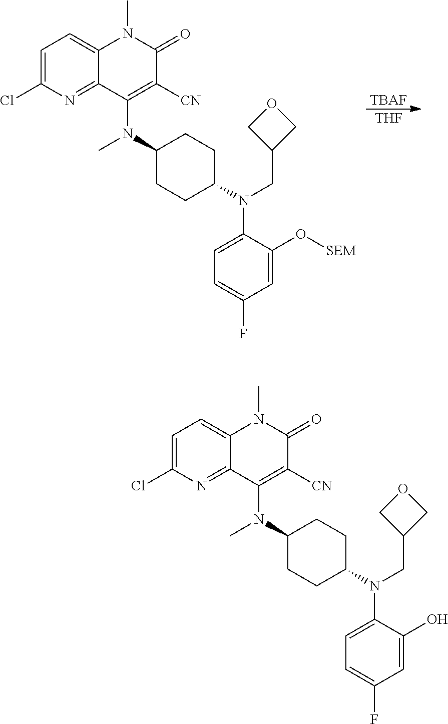 [SMILES: Cn1c(=O)c(C#N)c(N(C)[C@H]2CC[C@H](N(CC3COC3)c3ccc(F)cc3OCOCC[Si](C)(C)C)CC2)c2nc(Cl)ccc21 ; "TBAF" ; Cn1c(=O)c(C#N)c(N(C)[C@H]2CC[C@H](N(CC3COC3)c3ccc(F)cc3O)CC2)c2nc(Cl)ccc21 ; "THF"]

To a vial charged with trans-6-chloro-4-[[4-[4-fluoro-N-(oxetan-3-ylmethyl)-2-(2-trimethylsilylethoxymethoxy)anilino]cyclohexyl]-methyl-amino]-1-methyl-2-oxo-1,5-naphthyridine-3-carbonitrile (250 mg, 381 μmol) was added TBAF (1 M in THF, 2 mL), then heated to 50° C. for 12 h. Upon cooling to rt, it was diluted with water (20 mL) and extracted with EtOAc (3×20 mL). The combined organic layers were washed with brine (30 mL), dried over anhydrous sodium sulfate and concentrated under reduced pressure. The residue was purified by reverse flash chromatography (Column: C18 silica gel, 80 g, 20-35 um; Mobile Phase, A: water with 10 mmol/L NH₄HCO₃ and B: ACN, 5-60% in 50 min), affording the title compound (79.1 mg, 135 μmol, 35%) as a light-yellow solid.

¹H NMR (400 MHz, DMSO-d₆) δ 8.70 (br s, 1H), 8.04 (d, J=8.8 Hz, 1H), 7.78 (d, J=8.8 Hz, 1H), 7.11 (dd, J=8.7, 6.4 Hz, 1H), 6.61 (dd, J=10.4, 2.8 Hz, 1H) 6.56 (td, J=8.4, 2.8 Hz, 1H), 4.38 (dd, J=7.8, 5.9 Hz, 2H), 4.22-4.15 (m, 1H), 4.13 (t, J=6.1 Hz, 2H), 3.51 (s, 3H), 3.26 (d, J=7.6 Hz, 2H), 3.19 (s, 3H), 2.89-2.79 (m, 1H), 2.77-2.68 (m, 1H), 2.03-1.94 (m, 4H), 1.86-1.72 (m, 2H), 1.38-1.24 (m, 2H).

LCMS (ES, m/z): 526.15 [M+H]⁺.

### Example 30: Synthesis of cis-5-Methyl-8-((4-((oxetan-3-ylmethyl)(phenyl)amino) cyclohexyl) amino)-6-oxo-5,6-dihydro-1,5-naphthyridine-2-carbonitrile

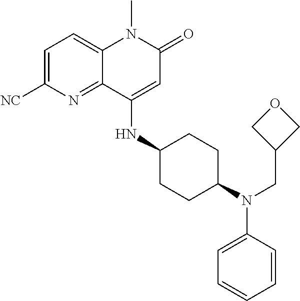 [SMILES: Cn1c(=O)cc(N[C@H]2CC[C@@H](N(CC3COC3)c3ccccc3)CC2)c2nc(C#N)ccc21]

### Step 1: cis/trans-Benzyl (4-(phenylamino)cyclohexyl)carbamate

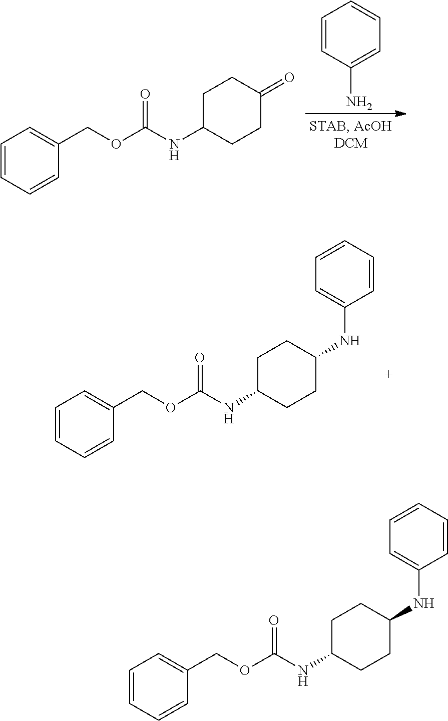

A solution of benzyl N-(4-oxocyclohexyl)carbamate (5.00 g, 20.2 mmol), aniline (1.88 g, 20.2 mmol, 1.84 mL), and acetic acid (243 mg, 4.04 mmol, 231 μL) in DCM (99.0 mL) was stirred for 0.25 h at rt. To the mixture was added sodium triacetoxyborohydride (8.57 g, 40.4 mmol), and the suspension was stirred for 1 h. The mixture was extracted with saturated sodium hydrogen carbonate (25 mL) and concentrated under reduced pressure. The crude product was purified by silica gel chromatography with ethyl acetate/heptane (0-50%), affording cis-benzyl (4-(phenylamino)cyclohexyl)carbamate (2.54 g, 39%) and trans-benzyl (4-(phenylamino) cyclohexyl) carbamate (1.95 g, 30%) as white solids (only the cis-isomer was used for Step 2).

LCMS (ES, m/z): 325.71 [M+H]⁺.

### Step 2: cis-Benzyl (4-((oxetan-3-ylmethyl)(phenyl)amino)cyclohexyl)carbamate

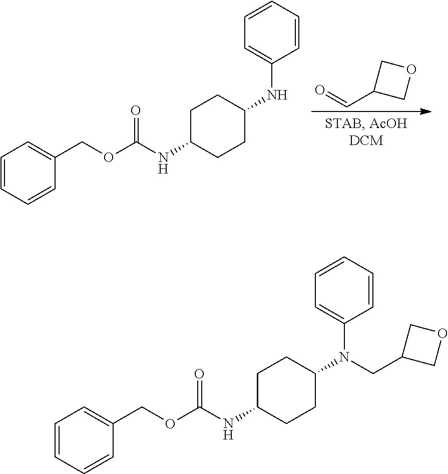 [SMILES: O=C(N[C@H]1CC[C@@H](Nc2ccccc2)CC1)OCc1ccccc1 ; O=CC1COC1 ; O=C(N[C@H]1CC[C@@H](N(CC2COC2)c2ccccc2)CC1)OCc1ccccc1]

A solution of cis-benzyl (4-(phenylamino)cyclohexyl)carbamate (80.0 mg, 247 μmol), oxetane-3-carbaldehyde (21.2 mg, 247 μmol), and acetic acid (3 mg, 49 μmol, 2.8 μL) in DCM (1.23 mL) was stirred for 0.25 h at rt. To the mixture was added sodium triacetoxyborohydride (105 mg, 493 μmol), and the suspension was stirred for 16 h. The mixture was extracted with saturated sodium hydrogen carbonate (0.5 mL) and concentrated under reduced pressure. The crude product was purified by silica gel chromatography (Ethyl acetate-Heptane, 0-50%), affording the title compound (58.1 mg, 60%) as an oil.

LCMS (ES, m/z): 395.38 [M+H]⁺.

### Step 3: cis-N-(Oxetan-3-ylmethyl)-N-phenylcyclohexane-1,4-diamine

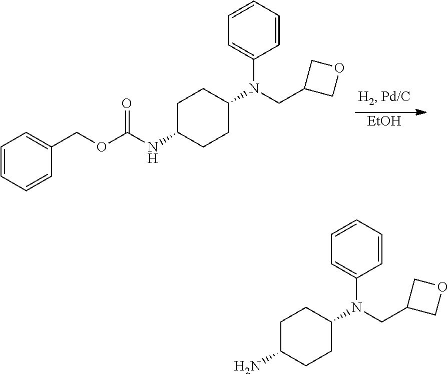 [SMILES: "EtOH" ; O=C(N[C@H]1CC[C@@H](N(CC2COC2)c2ccccc2)CC1)OCc1ccccc1 ; N[C@H]1CC[C@@H](N(CC2COC2)c2ccccc2)CC1]

A solution of cis-benzyl (4-((oxetan-3-ylmethyl)(phenyl)amino)cyclohexyl)carbamate (57.0 mg, 144 μmol), palladium (15.4 mg, 144 μmol, 10% on carbon), and ethanol (722 μL) were stirred under an atmosphere of hydrogen for 16 h at rt. The suspension was filtered and concentrated under reduced pressure to afford the crude product (37.2 mg, 99%) as an oil.

LCMS (ES, m/z): 261.40 [M+H]⁺.

### Step 4: cis-5-Methyl-8-((4-((oxetan-3-ylmethyl)(phenyl)amino)cyclohexyl)amino)-6-oxo-5,6-dihydro-1,5-naphthyridine-2-carbonitrile

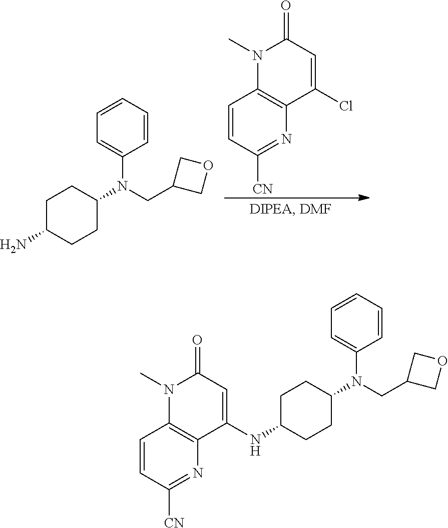

A solution of cis-N-(oxetan-3-ylmethyl)-N-phenylcyclohexane-1,4-diamine (37.2 mg, 143 μmol), 8-chloro-5-methyl-6-oxo-5,6-dihydro-1,5-naphthyridine-2-carbonitrile (31.4 mg, 143 μmol, prepared according to WO20202006016), and DIPEA (92.3 mg, 714 μmol, 124 μL) in DMF (828 μL) was stirred for 16 h at 80° C. The mixture was purified by reversed phase chromatography (Column: SunFire Prep OBD C18, 19×250 mm, 5 μm; Mobile Phase, A: water (0.1% formic acid) and B: ACN (0.1% formic acid, 10-40% in 10 min); Detector: UV254/220 nm). The product fractions were lyophilized to afford the title compound (6.2 mg, 9.6%) as a white solid.

¹H-NMR (CDCl₃, 300 MHz) δ (ppm): 7.85 (d, J=8.8 Hz, 1H), 7.67 (d, J=8.5 Hz, 1H), 7.31-7.23 (m, 2H), 6.96-6.87 (m, 3H), 6.48 (br d, J=7.2 Hz, 1H), 5.82 (s, 1H), 4.69 (t, J=6.8 Hz, 2H), 4.37 (t, J=5.8 Hz, 2H), 3.76-3.66 (m, 1H), 3.62 (s, 3H), 3.48 (br d, J=7.1 Hz, 3H), 3.21-3.09 (m, 1H), 2.16-2.00 (m, 2H), 1.82-1.69 (m, 6H).

LCMS (ES, m/z): 444.47 [M+H]⁺.

### Example 3R: Synthesis of trans-8-((4-((Cyclopropylmethyl)(4-fluorophenyl)amino) cyclohexyl) amino)-5-methyl-6-oxo-5,6-dihydro-1,5-naphthyridine-2-carbonitrile

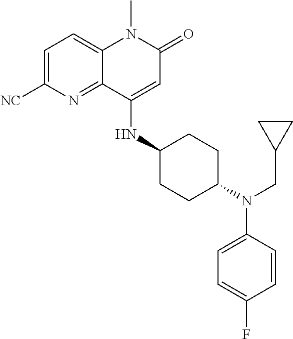 [SMILES: Cn1c(=O)cc(N[C@H]2CC[C@H](N(CC3CC3)c3ccc(F)cc3)CC2)c2nc(C#N)ccc21]

### Step 1: trans-Benzyl (4-((cyclopropylmethyl)(4-fluorophenyl)amino)cyclohexyl) carbamate

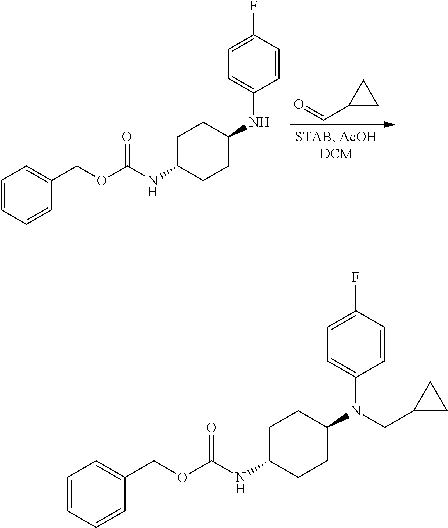

The compound was prepared with the same method as example 3Q, using trans-benzyl (4-((4-fluorophenyl)amino)cyclohexyl)carbamate (337 mg, 983 μmol), cyclopropane-carbaldehyde (138 mg, 1.97 mmol, 147 μL), DCM (4.3 mL), acetic acid (11.8 mg, 197 μmol, 11.3 μL), and sodium triacetoxyborohydride (463 mg, 1.97 mmol), to afford the title compound (332 mg, 85%) as an oil.

LCMS (ES, m/z): 397.46 [M+H]⁺.

### Step 2: trans-N-(Cyclopropylmethyl)-N-(4-fluorophenyl)cyclohexane-1,4-diamine

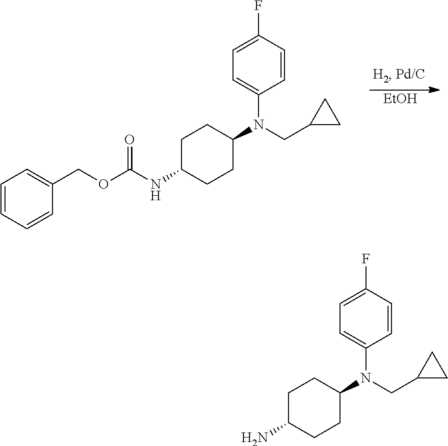 [SMILES: "EtOH" ; O=C(N[C@H]1CC[C@H](N(CC2CC2)c2ccc(F)cc2)CC1)OCc1ccccc1 ; N[C@H]1CC[C@H](N(CC2CC2)c2ccc(F)cc2)CC1]

The compound was prepared with the same method as Example 3Q, using trans-benzyl (4-((cyclopropylmethyl)(4-fluorophenyl)amino)cyclohexyl)carbamate (330 mg, 832 μmol), palladium (10% on carbon, 88.6 mg), ethanol (5.6 mL), and hydrogen, to afford the title compound (199 mg, 91%) as an oil.

LCMS (ES, m/z): 263.43 [M+H]⁺.

### Step 3: trans-8-((4-((Cyclopropylmethyl)(4-fluorophenyl)amino)cyclohexyl)amino)-5-methyl-6-oxo-5,6-dihydro-1,5-naphthyridine-2-carbonitrile

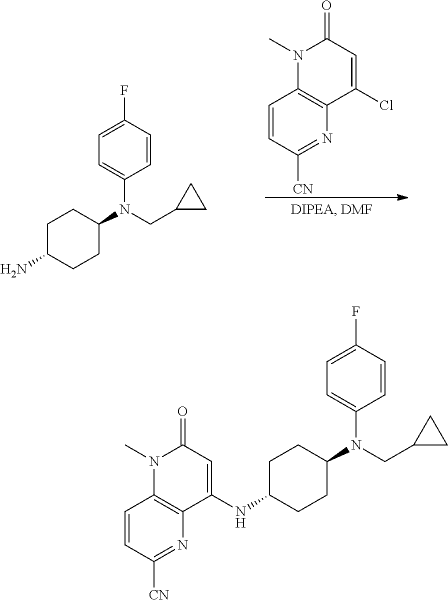 [SMILES: N[C@H]1CC[C@H](N(CC2CC2)c2ccc(F)cc2)CC1 ; Cn1c(=O)cc(Cl)c2nc(C#N)ccc21 ; Cn1c(=O)cc(N[C@H]2CC[C@H](N(CC3CC3)c3ccc(F)cc3)CC2)c2nc(C#N)ccc21]

The compound was prepared with the same method as Example 3Q, using trans-N-(cyclopropylmethyl)-N-(4-fluorophenyl)cyclohexane-1,4-diamine (85.0 mg, 324 μmol), DMF (1.3 mL), DIPEA (209 mg, 1.62 mmol, 282 μL), and 8-chloro-5-methyl-6-oxo-5,6-dihydro-1,5-naphthyridine-2-carbonitrile (71.2 mg, 324 μmol), and purified by reversed phase chromatography (Column: SunFire Prep OBD C18, 19×250 mm, 5 μm; Mobile Phase, A: water (0.1% formic acid) and B: ACN (0.1% formic acid, 5-40% in 10 min); Detector: UV254/220 nm), to afford the title compound (9.7 mg, 6.6%) as a white solid.

¹H-NMR (CDCl₃, 300 MHz) δ (ppm): 7.81 (d, J=8.7 Hz, 1H), 7.64 (d, J=8.9 Hz, 1H), 7.01-6.91 (m, 2H), 6.91-6.83 (m, 2H), 6.23 (d, J=7.6 Hz, 1H), 5.81 (s, 1H), 3.61 (s, 3H), 3.54-3.43 (m, 1H), 3.41-3.24 (m, 1H), 3.03 (d, J=5.9 Hz, 2H), 2.28 (br d, J=10.7 Hz, 2H), 2.06-1.95 (br d, J=10.7 Hz, 2H), 1.58-1.37 (m, 4H), 0.99-0.84 (m, 1H), 0.57-0.48 (m, 2H), 0.23-0.15 (m, 2H).

LCMS (ES, m/z): 446.54 [M+H]⁺.

### Example 3S: Synthesis of cis-8-((4-((Cyclopropylmethyl)(4-fluorophenyl)amino) cyclohexyl) amino)-5-methyl-6-oxo-5,6-dihydro-1,5-naphthyridine-2-carbonitrile

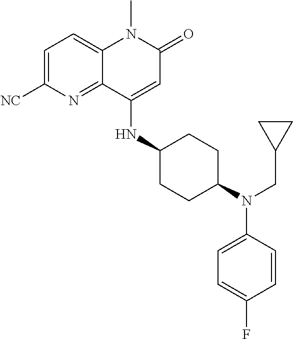 [SMILES: Cn1c(=O)cc(N[C@H]2CC[C@@H](N(CC3CC3)c3ccc(F)cc3)CC2)c2nc(C#N)ccc21]

### Step 1: cis trans-benzyl (4-((4-fluorophenyl)amino)cyclohexyl)carbamate

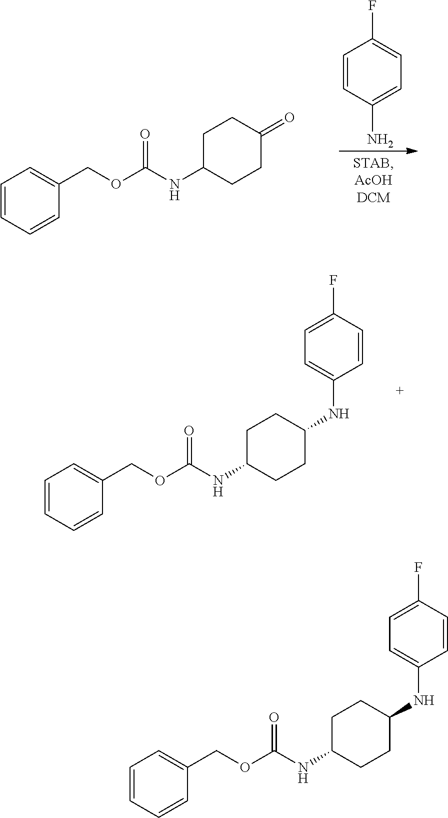 [SMILES: O=C1CCC(NC(=O)OCc2ccccc2)CC1 ; Nc1ccc(F)cc1 ; O=C(N[C@H]1CC[C@@H](Nc2ccc(F)cc2)CC1)OCc1ccccc1 ; O=C(N[C@H]1CC[C@H](Nc2ccc(F)cc2)CC1)OCc1ccccc1]

The compound was prepared with the same method as Example 3Q, using benzyl N-(4-oxocyclohexyl)carbamate (5.01 g, 20.3 mmol), 4-fluoroaniline (2.25 g, 20.3 mmol, 1.94 mL), DCM (99 mL), acetic acid (243 mg, 4.05 mmol, 232 μL) and sodium triacetoxyborohydride (8.59 g, 40.5 mmol), to afford cis-trans-benzyl (4-((4-fluorophenyl)amino)cyclohexyl)-carbamate (3.19 g, 46%) and trans-benzyl (4-((4-fluorophenyl)amino)cyclohexyl)carbamate (1.90 g, 27%) as white solids (the cis-isomer was used for Step 2).

cis: LCMS (ES, m/z): 343.39 [M+H]⁺.

trans: LCMS (ES, m/z): 343.43 [M+H]⁺.

### Step 2: cis-Benzyl (4-((cyclopropylmethyl)(4-fluorophenyl)amino)cyclohexyl)carbamate

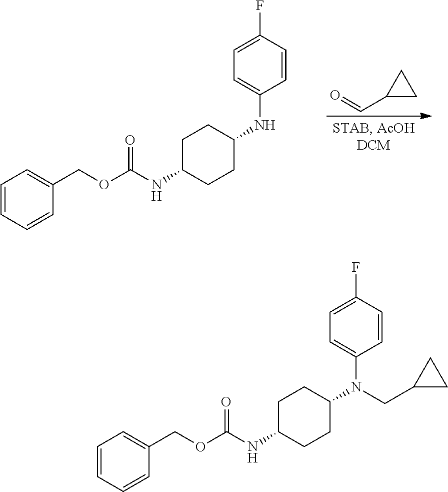 [SMILES: O=C(N[C@H]1CC[C@@H](Nc2ccc(F)cc2)CC1)OCc1ccccc1 ; O=CC1CC1 ; O=C(N[C@H]1CC[C@@H](N(CC2CC2)c2ccc(F)cc2)CC1)OCc1ccccc1]

The compound was prepared with the same method as Example 3Q, using cis-benzyl (4-((4-fluorophenyl)amino)cyclohexyl)carbamate (336 mg, 983 μmol), cyclopropanecarbaldehyde (138 mg, 1.97 mmol, 147 μL), DCM (4.3 mL), acetic acid (11.8 mg, 197 μmol, 11.3 μL), and sodium triacetoxyborohydride (463 mg, 1.97 mmol), to afford the title compound (298 mg, 77%) as an oil.

LCMS (ES, m/z): 397.46 [M+H]⁺.

### Step 3: cis-N-(Cyclopropylmethyl)-N-(4-fluorophenyl)cyclohexane-1,4-diamine

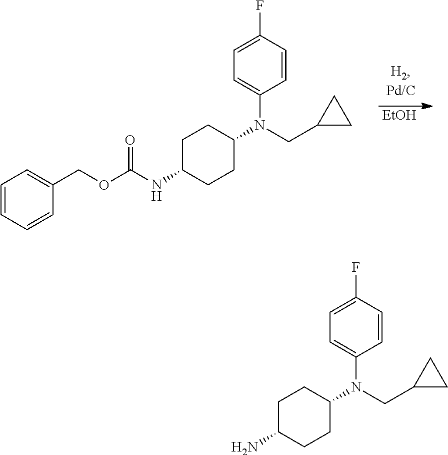 [SMILES: "EtOH" ; O=C(N[C@H]1CC[C@@H](N(CC2CC2)c2ccc(F)cc2)CC1)OCc1ccccc1 ; N[C@H]1CC[C@@H](N(CC2CC2)c2ccc(F)cc2)CC1]

The compound was prepared with the same method as Example 3Q, using cis-benzyl (4-((cyclopropylmethyl)(4-fluorophenyl)amino)cyclohexyl)carbamate (295 mg, 744.02 μmol), palladium (10% on carbon, 79.2 mg), ethanol (5 mL), and hydrogen, to afford the title compound (194 mg, 99%) as an oil.

LCMS (ES, m/z): 263.39 [M+H]+.

### Step 4: cis-8-((4-((Cyclopropylmethyl)(4-fluorophenyl)amino)cyclohexyl)amino)-5-methyl-6-oxo-5,6-dihydro-1,5-naphthyridine-2-carbonitrile

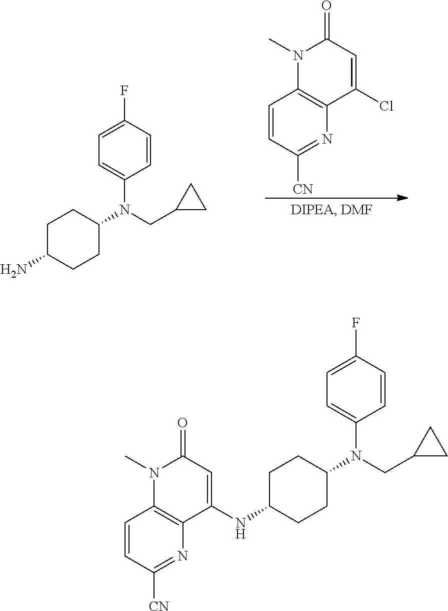 [SMILES: N[C@H]1CC[C@@H](N(CC2CC2)c2ccc(F)cc2)CC1 ; Cn1c(=O)cc(Cl)c2nc(C#N)ccc21 ; Cn1c(=O)cc(N[C@H]2CC[C@@H](N(CC3CC3)c3ccc(F)cc3)CC2)c2nc(C#N)ccc21]

The compound was prepared with the same method as Example 3Q, using cis-N-(cyclopropylmethyl)-N-(4-fluorophenyl)cyclohexane-1,4-diamine (94.9 mg, 318 μmol), DMF (1.3 mL), and DIPEA (205 mg, 1.59 mmol, 277 μL), and 8-chloro-5-methyl-6-oxo-5,6-dihydro-1,5-naphthyridine-2-carbonitrile (69.8 mg, 318 μmol), and purified by reversed phase chromatography (Column: SunFire Prep OBD C18, 19×250 mm, 5 μm; Mobile Phase, A: water (0.1% formic acid) and B: ACN (0.1% formic acid, 5-40% in 10 min); Detector: UV254/220 nm), to afford the title compound (1.2 mg, 8.3%) as a white solid.

¹H-NMR (CDCl₃, 300 MHz) δ (ppm): 7.83 (d, J=8.3 Hz, 1H), 7.65 (d, J=8.6 Hz, 1H), 7.01-6.89 (m, 4H), 6.50 (d, J=6.6 Hz, 1H), 5.81 (s, 1H), 3.76-3.66 (m, 1H), 3.61 (s, 3H), 3.53-3.43 (m, 1H), 3.03 (d, J=5.9 Hz, 2H), 2.13-1.97 (m, 2H), 1.81-1.68 (m, 6H), 0.94-0.78 (m, 1H), 0.55-0.45 (m, 2H), 0.18-0.12 (m, 2H).

LCMS (ES, m/z): 446.50 [M+H]⁺.

### Example 3T: Synthesis of trans-8-((4-(cyclopropyl(phenyl)amino)cyclohexyl)(methyl)amino)-5-methyl-6-oxo-5,6-dihydro-1,5-naphthyridine-2-carbonitrile

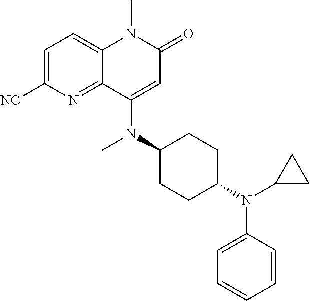 [SMILES: Cn1c(=O)cc(N(C)[C@H]2CC[C@H](N(c3ccccc3)C3CC3)CC2)c2nc(C#N)ccc21]

### Step 1: trans-N¹-Cyclopropyl-N¹-phenylcyclohexane-1,4-diamine

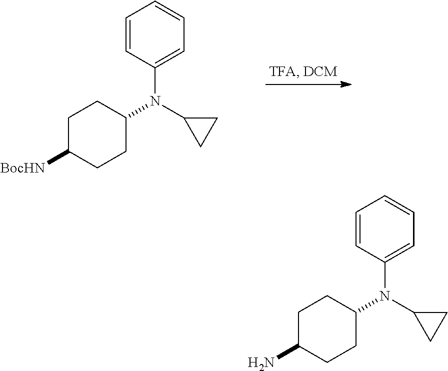

A solution of trans-tert-butyl N-[4-(N-cyclopropylanilino)cyclohexyl]carbamate (1.3 g, 3.93 mmol) in DCM (12 mL) and TFA (3 mL) was stirred at room temperature for 2 h. The mixture was basified to pH 8 with saturated NaHCO₃(aq) at 0° C. and extracted with DCM (3×40 mL). The combined organic layers were dried over anhydrous sodium sulfate and concentrated under reduced pressure to afford the title compound (850 mg, 3.69 mmol, 94%) as a yellow solid.

LCMS (ES, m/z): 231.35 [M+H]⁺

### Step 2: trans-8-[[4-(N-Cyclopropylanilino)cyclohexyl]amino]-5-methyl-6-oxo-1,5-naphthyridine-2-carbonitrile

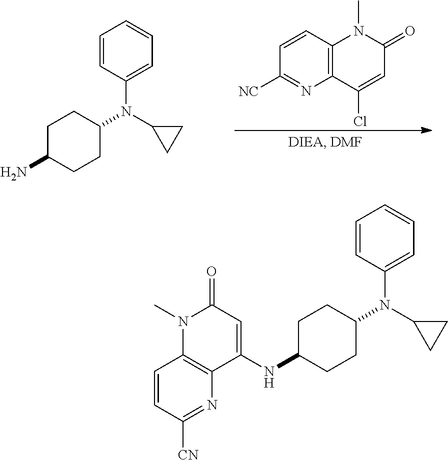

A solution of trans-N¹-cyclopropyl-N¹-phenylcyclohexane-1,4-diamine (157 mg, 683 μmol), 8-chloro-5-methyl-6-oxo-1,5-naphthyridine-2-carbonitrile (150 mg, 683 μmol) and DIEA (265 mg, 2.05 mmol) in DMF (5 mL) was stirred at 120° C. for 12 h under nitrogen atmosphere. The mixture was cooled to rt, diluted with water (15 mL) and extracted with EtOAc (3×15 mL). The combined organic layers were washed with brine (20 mL), dried over anhydrous sodium sulfate and concentrated under reduced pressure. The residue was purified by silica gel chromatography (petroleum ether-EtOAc, 2:1) to afford the title compound (90 mg, 218 μmol, 32%) as a light-yellow solid.

LCMS (ES, m/z): 414.40 [M+H]⁺

### Step 3: trans-8-[[4-(N-Cyclopropylanilino)cyclohexyl]-methyl-amino]-5-methyl-6-oxo-1,5-naphthyridine-2-carbonitrile

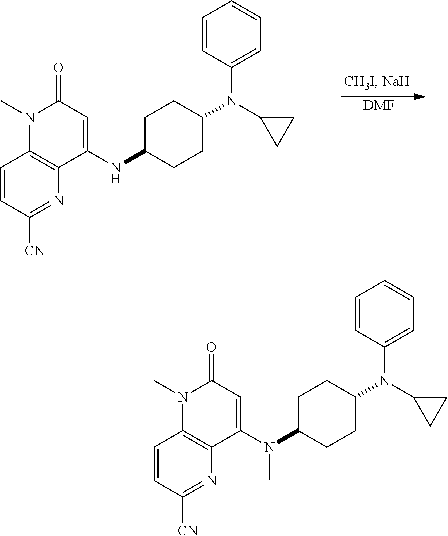

To a stirred solution of trans-8-[[4-(N-cyclopropylanilino)cyclohexyl]amino]-5-methyl-6-oxo-1,5-naphthyridine-2-carbonitrile (90 mg, 218 μmol) in DMF (2 mL) was added NaH (17 mg, 435 μmol, 60% in min. oil) at 0° C. After 30 min, iodomethane (93 mg, 653 μmol) was added, drop-wise. The resulting mixture was stirred at 0° C. for 1.5 h, quenched with saturated NH₄Cl (aq, 10 mL) and extracted with EtOAc (3×10 mL). The combined organic layers were washed with brine (10 mL), dried over anhydrous sodium sulfate and concentrated under reduced pressure. The residue was purified by reverse flash chromatography (Column: C18 silica gel, 40 g, 20-35 um; Mobile Phase, A: water (10 mmol/L NH₄HCO₃) and B: ACN (20% to 75% in 30 min); Detector: 254 nm), affording the title compound (37.8 mg, 88.4 μmol, 41%) as a light-yellow solid.

¹H NMR (400 MHz, DMSO-d₆) δ 8.16 (d, J=8.8 Hz, 1H), 8.04 (d, J=8.9 Hz, 1H), 7.22-7.14 (m, 2H), 7.01 (d, J=6.0 Hz, 2H), 6.74 (t, J=5.4 Hz, 1H), 5.87 (s, 1H), 4.43-4.35 (m, 1H), 3.56-3.48 (m, 4H), 2.85 (s, 3H), 2.33-2.27 (m, 1H), 1.96-1.71 (m, 8H), 0.83-0.75 (m, 2H), 0.39-0.33 (m, 2H).

LCMS (ES, m/z): 428.25 [M+H]⁺

### Example 3U: Synthesis of cis-8-[[4-(N-Cyclopropyl-4-fluoro-2-methoxy-anilino)cyclohexyl]-methyl-amino]-5-methyl-6-oxo-1,5-naphthyridine-2-carbonitrile

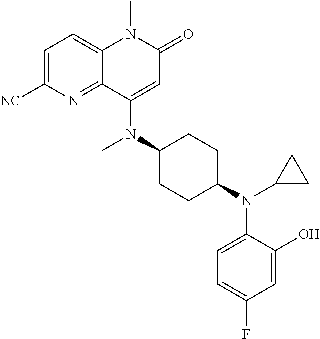 [SMILES: Cn1c(=O)cc(N(C)[C@H]2CC[C@@H](N(c3ccc(F)cc3O)C3CC3)CC2)c2nc(C#N)ccc21]

### Step 1: (2-((2-Bromo-5-fluorophenoxy)methoxy)ethyl)trimethylsilane

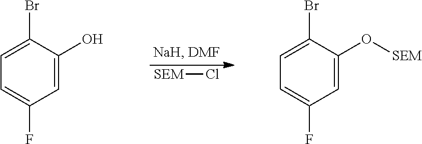 [SMILES: Oc1cc(F)ccc1Br ; C[Si](C)(C)CCOCCl ; C[Si](C)(C)CCOCOc1cc(F)ccc1Br]

To a stirred solution of 2-bromo-5-fluoro-phenol (10 g, 52.4 mmol, 5.82 mL) in DMF (207 mL) at 0° C. was added NaH (4.19 g, 104.8 mmol, 60% in min. oil). To the mixture, after 0.5 h was added SEM-Cl (10.47 g, 62.8 mmol, 11.1 mL). The resulting mixture was stirred at rt for 1.5 h, then diluted with ice-water (400 mL) and extracted with EtOAc (3×300 mL). The combined organic layers were washed with brine (800 mL), dried over anhydrous sodium sulfate and concentrated under reduced pressure. The crude residue purified by silica gel chromatography (EtOAc-Petroleum ether, 10%), to afford the title compound (16 g, 95%) as a colorless oil.

### Step 2: cis-tert-Butyl N-[4-[4-fluoro-2-(2-trimethylsilylethoxymethoxy) anilino]cyclohexyl]carbamate

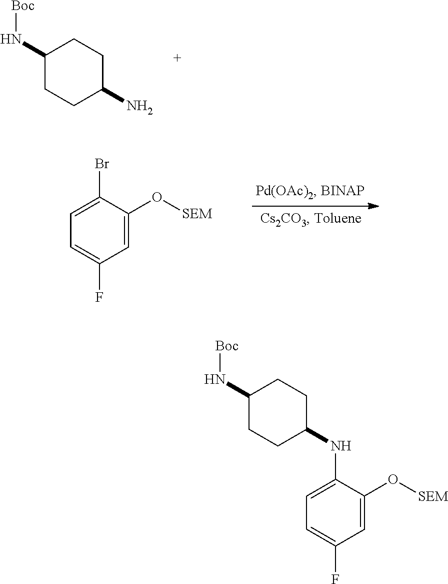

A solution of cis-tert-butyl N-(4-aminocyclohexyl)carbamate (7.0 g, 32.7 mmol), 2-[(2-bromo-5-fluoro-phenoxy)methoxy]ethyl-trimethyl-silane (15.74 g, 49.0 mmol), BINAP (4.07 g, 6.53 mmol), Pd(OAc)₂ (1.10 g, 4.90 mmol) and Cs₂CO₃ (31.93 g, 97.99 mmol) in toluene (180 mL) was stirred at 110° C. for 12 h under a nitrogen atmosphere. The mixture was cooled to rt, diluted with water (100 mL) and extracted with EtOAc (3×100 mL). The combined organic layers were washed with brine (100 mL), dried over anhydrous sodium sulfate and concentrated under reduced pressure. The residue was purified by silica gel column chromatography (Petroleum ether-EtOAc, 5:1) to afford the title compound (6 g, 13.20 mmol, 40%) as a yellow oil.

LCMS (ES, m/z): 455.30 [M+H]⁺

### Step 3: cis-tert-Butyl N-[4-[N-cyclopropyl-4-fluoro-2-(2-trimethylsilylethoxymethoxy) anilino]cyclohexyl]carbamate

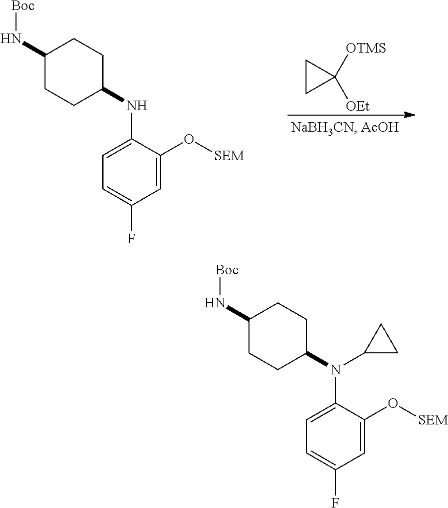

A solution of cis-tert-butyl N-[4-[4-fluoro-2-(2-trimethylsilylethoxymethoxy) anilino]cyclohexyl]carbamate (4 g, 8.8 mmol), (1-methoxycyclopropoxy)-trimethylsilane (7.05 g, 44.0 mmol) and NaBH₃CN (2.21 g, 35.2 mmol) in AcOH (36 mL) was stirred for 1.5 h at 50° C. After cooling to rt, the mixture was diluted with water (50 mL) and extracted with EtOAc (3×50 mL). The combined organic layers were washed with brine (50 mL), dried over anhydrous sodium sulfate and concentrated under reduced pressure. The crude residue was purified by silica gel column chromatography (Petroleum ether-EtOAc, 6:1) to afford the title compound (3.15 g, 6.37 mmol, 72%) as a yellow oil.

LCMS (ES, m/z): 495.30 [M+H]⁺

### Step 4: cis-tert-Butyl-N-[4-[N-cyclopropyl-4-fluoro-2-(2-trimethylsilylethoxymethoxy) anilino]cyclohexyl]-N-methyl-carbamate

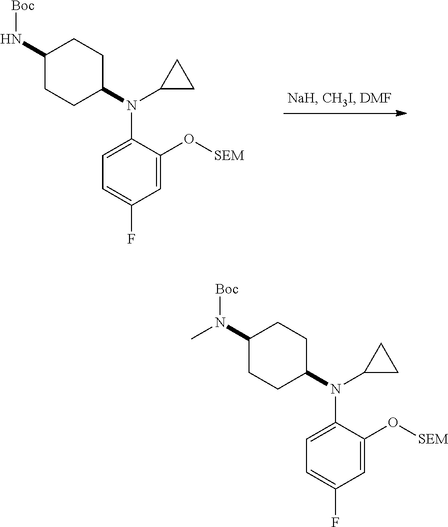

To a solution of cis-tert-butyl-N-[4-[N-cyclopropyl-4-fluoro-2-(2-trimethylsilylethoxymethoxy) anilino]cyclohexyl]carbamate (3.15 g, 6.37 mmol) in DMF (30 mL) at 0° C. was added NaH (1.02 g, 25.5 mmol, 60% in min. oil). To the mixture, after 30 min was added iodomethane (4.52 g, 31.84 mmol), drop-wise. The resulting mixture was stirred at 0° C. for 1.5 h, then quenched with saturated NH₄Cl (50 mL, aq) and extracted with EtOAc (3×50 mL). The combined organic layers were washed with brine (50 mL), dried over anhydrous sodium sulfate and concentrated under reduced pressure. The crude residue was purified by silica gel column chromatography (Petroleum ether-EtOAc, 8:1) to afford the title compound (2.5 g, 4.9 mmol, 77%) as a colorless oil.

LCMS (ES, m/z): 509.45 [M+H]⁺

### Step 5: cis-2-[Cyclopropyl-[4-(methylamino)cyclohexyl]amino]-5-fluoro-phenol

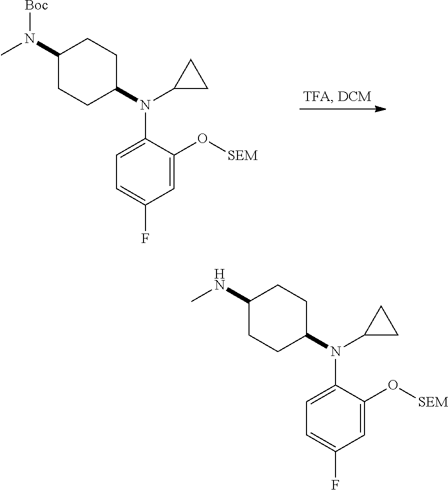

To a solution of tert-butyl N-[4-[N-cyclopropyl-4-fluoro-2-(2-trimethylsilylethoxymethoxy) anilino]cyclohexyl]-N-methyl-carbamate (2.5 g, 4.91 mmol) in DCM (30 mL) was added TFA (5.5 mL). After 2 h at rt, it was concentrated under reduced pressure, basified to pH 8 with saturated NaHCO₃(aq) at 0° C. and extracted with EtOAc (3×50 mL). The combined organic layers were washed with brine (50 mL), dried over anhydrous sodium sulfate and concentrated under reduced pressure to afford the title compound (830 mg, 2.98 mmol, 61%) as a yellow oil.

LCMS (ES, m/z): 279.20 [M+H]⁺

### Step 6: cis-8-[[4-(N-Cyclopropyl-4-fluoro-2-hydroxy-anilino)cyclohexyl]-methyl-amino]-5-methyl-6-oxo-1,5-naphthyridine-2-carbonitrile

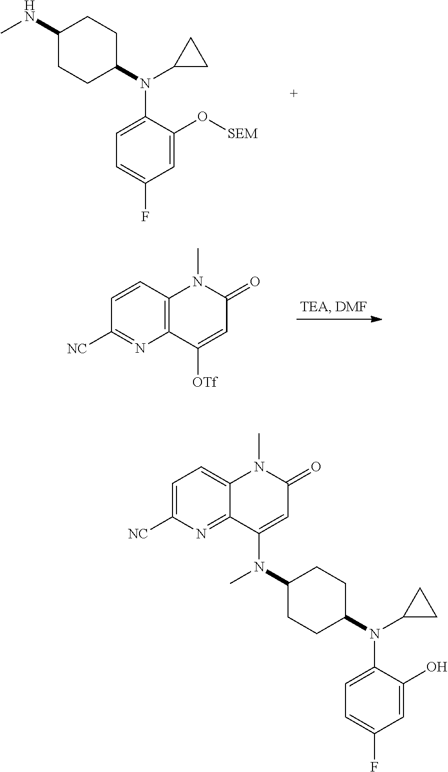

A solution of cis-2-[cyclopropyl-[4-(methylamino)cyclohexyl]amino]-5-fluoro-phenol (200 mg, 718.48 μmol), (6-cyano-1-methyl-2-oxo-1,5-naphthyridin-4-yl) trifluoromethanesulfonate (239 mg, 718.5 μmol, prepared according to WO2020006018) and TEA (363 mg, 3.59 mmol) in DMF (3 mL) was stirred for 2 h at 80° C. The mixture was cooled and purified by reverse flash chromatography (Column: C18 silica gel, 20-35 um; Mobile Phase, A: water with 10 mmol/L NH₄HCO₃, and B: ACN, 5-80% over 50 min; Detector: 254 nm). The collected fraction was lyophilized to afford the title compound (50.2 mg, 106.8 μmol, 15%) as a light-yellow solid.

¹H NMR (300 MHz, DMSO-d₆) δ 8.80 (s, 1H), 8.13 (d, J=8.8 Hz, 1H), 8.02 (d, J=8.9 Hz, 1H), 7.18 (dd, J=8.7, 6.5 Hz, 1H), 6.68-6.51 (m, 2H), 5.85 (s, 1H), 4.37-4.25 (m, 1H), 3.50 (s, 3H), 3.31 (s, 1H), 2.89 (s, 3H), 2.34 (br s, 1H), 2.15-1.80 (m, 4H), 1.52-1.33 (m, 4H), 0.60-0.33 (m, 4H).

LCMS (ES, m/z): 462.25 [M+H]⁺

### Example 3V: Synthesis of trans-8-[[4-(N-Cyclopropyl-4-fluoro-2-methoxy-anilino)cyclohexyl-methyl-amino]-5-methyl-6-oxo-1,5-naphthyridine-2-carbonitrile

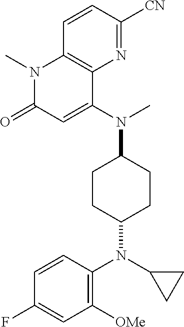 [SMILES: COc1cc(F)ccc1N(C1CC1)[C@H]1CC[C@H](N(C)c2cc(=O)n(C)c3ccc(C#N)nc23)CC1]

Prepared in a similar manner to the examples described herein. The final product was purified by reverse flash chromatography (Column: C18 silica gel, 80 g, 20-35 um; Mobile phase, A: water (0.1% NH₄CO₃) and B: ACN (10% to 70% in 30 min); Detector: UV 220 nm.) to afford the title compound (28.6 mg, 8.8%) as a light-yellow solid.

¹H NMR (300 MHz, MeOH-d₄) δ 8.08-7.95 (m, 2H), 7.10 (dd, J=8.7, 6.4 Hz, 1H), 6.72 (dd, J=10.8, 2.8 Hz, 1H), 6.59 (td, J=8.4, 2.8 Hz, 1H), 5.93 (s, 1H), 4.60-4.49 (m, 1H), 3.84 (s, 3H), 3.64 (s, 3H), 3.26-3.17 (m, 1H), 2.94 (s, 3H), 2.60 (tt, J=6.6, 3.7 Hz, 1H), 2.06-1.94 (m, 4H), 1.81-1.60 (m, 4H), 0.62-0.54 (m, 2H), 0.36-0.28 (m, 2H). LCMS (ES, m/z): 476.30 [M+H]⁺

### Example 3W: Synthesis of cis-8-[[4-(N-Cyclopropyl-4-fluoro-2-methoxy-anilino)cyclohexyl]-methyl-amino]-5-methyl-6-oxo-1,5-naphthyridine-2-carbonitrile

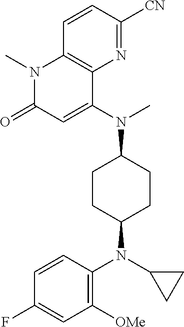 [SMILES: COc1cc(F)ccc1N(C1CC1)[C@H]1CC[C@@H](N(C)c2cc(=O)n(C)c3ccc(C#N)nc23)CC1]

### Step 1: cis-tert-Butyl N-[4-(4-fluoro-2-methoxy-anilino)cyclohexyl]carbamate

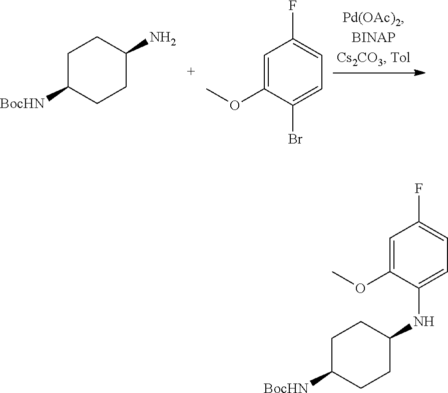

Prepared in a similar manner to Example 1U using cis-tert-butyl N-(4-aminocyclohexyl) carbamate (4 g, 18.7 mmol), 1-bromo-4-fluoro-2-methoxy-benzene (9.95 g, 48.5 mmol), BINAP (2.32 g, 3.73 mmol), Pd(OAc)₂ (838 mg, 3.73 mmol) and Cs₂CO₃ (18.24 g, 56.0 mmol) in toluene (100 mL), affording the title compound (1.9 g, 5.61 mmol, 30%) as a yellow solid.

LCMS (ES, m/z): 339.40 [M+H]⁺

### Step 2: cis-tert-butyl N-[4-(N-cyclopropyl-4-fluoro-2-methoxy-anilino)cyclohexyl]carbamate

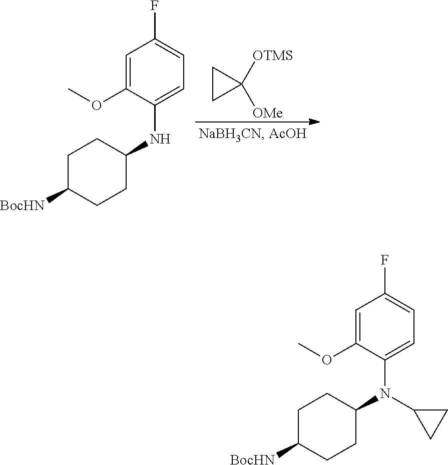

Prepared in a similar manner to Example 1U using cis-butyl N-[4-(4-fluoro-2-methoxy-anilino)cyclohexyl]carbamate (1.9 g, 5.61 mmol), (1-methoxycyclopropoxy)-trimethyl-silane (4.50 g, 28.1 mmol) and NaBH₃CN (1.41 g, 22.5 mmol) in AcOH (25 mL), affording the title compound (1.9 g, 5.02 mmol, 89%) as a white solid.

LCMS (ES, m/z): 379.25 [M+H]⁺

### Step 3: cis-tert-butyl N-[4-(N-cyclopropyl-4-fluoro-2-methoxy-anilino)cyclohexyl]-N-methyl-carbamate

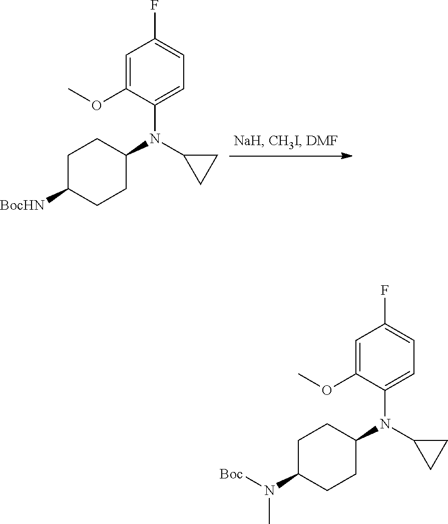 [SMILES: COc1cc(F)ccc1N(C1CC1)[C@H]1CC[C@@H](NC(=O)OC(C)(C)C)CC1 ; COc1cc(F)ccc1N(C1CC1)[C@H]1CC[C@@H](N(C)C(=O)OC(C)(C)C)CC1]

Prepared in a similar manner to Example 1U using cis-tert-butyl N-[4-(N-cyclopropyl-4-fluoro-2-methoxy-anilino)cyclohexyl]carbamate (1.9 g, 5.02 mmol), NaH (401 mg, 10.04 mmol, 60% in min. oil) and Mel (2.85 g, 20.1 mmol) in DMF (25 mL), affording the title compound (1.6 g, 4.08 mmol, 81%) as a white solid.

LCMS (ES, m/z): 393.25 [M+H]⁺

### Step 4: cis-N-4-cyclopropyl-N4-(4-fluoro-2-methoxy-phenyl)-N1-methyl-cyclohexane-1,4-diamine; 2,2,2-trifluoroacetic acid

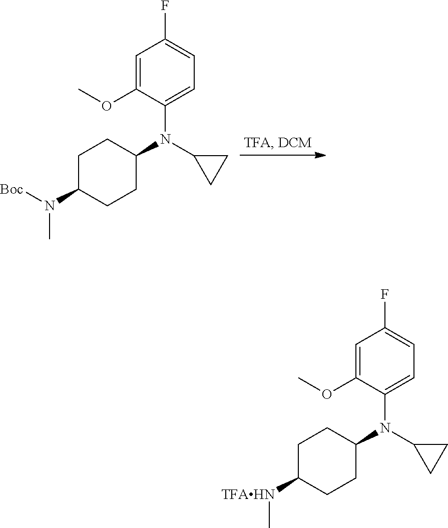

Prepared in a similar manner to Example 1U using cis-tert-butyl N-[4-(N-cyclopropyl-4-fluoro-2-methoxy-anilino)cyclohexyl]-N-methyl-carbamate (1.6 g, 4.08 mmol) in DCM (10 mL) and TFA (2.5 mL), affording the title compound (1.6 g, assumed quantitative) as a brown oil.

LCMS (ES, m/z): 293.20 [M-TFA+H]⁺

### Step 5: cis-8-[[4-(N-cyclopropyl-4-fluoro-2-methoxy-anilino)cyclohexyl]-methyl-amino]-5-methyl-6-oxo-1,5-naphthyridine-2-carbonitrile

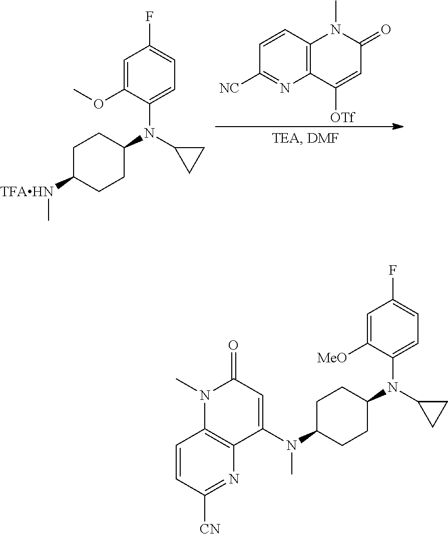

Prepared in a similar manner to Example 1U using cis-N4-cyclopropyl-N4-(4-fluoro-2-methoxy-phenyl)-N₁-methyl-cyclohexane-1,4-diamine (316 mg, 1.08 mmol), (6-cyano-1-methyl-2-oxo-1,5-naphthyridin-4-yl) trifluoromethanesulfonate (240 mg, 720 μmol) and TEA (364 mg, 3.60 mmol) in DMF (4 mL). The residue was purified by reverse flash chromatography (Column: C18 silica gel, 80 g, 20-35 um; Mobile Phase, A: water (10 mmol/L NH₄HCO₃) and B: ACN (5% to 80% in 50 min); Detector: 254 nm). The collected fraction was lyophilized to afford the title compound (54 mg, 110 μmol, 15%, 97.3% purity) as an off-white solid.

¹H NMR (400 MHz, Methanol-d₄) δ 8.03 (d, J=8.9 Hz, 1H), 7.96 (d, J=8.8 Hz, 1H), 7.08 (dd, J=8.5, 6.6 Hz, 1H), 6.76 (dd, J=10.9, 2.8 Hz, 1H), 6.59 (td, J=8.3, 2.8 Hz, 1H), 5.94 (s, 1H), 4.63-4.50 (m, 1H), 3.83 (s, 3H), 3.63 (s, 3H), 3.57-3.52 (m, 1H), 3.01 (s, 3H), 2.46 (p, J=5.5 Hz, 1H), 2.16-1.99 (m, 4H), 1.58-1.43 (m, 4H), 0.50-0.38 (m, 4H).

LCMS (ES, m/z): 476.20 [M+H]⁺

### Example 3X: Synthesis of trans-8-((4-(cyclopropyl(4-fluoro-2-hydroxyphenyl)amino) cyclohexyl)(methyl)amino)-5-methyl-6-oxo-5,6-dihydro-1,5-naphthyridine-2-carbonitrile

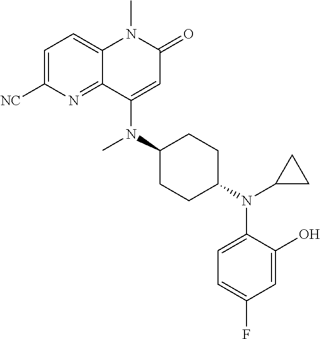 [SMILES: Cn1c(=O)cc(N(C)[C@H]2CC[C@H](N(c3ccc(F)cc3O)C3CC3)CC2)c2nc(C#N)ccc21]

### Step 1: 2-[(2-Bromo-5-fluoro-phenoxy)methoxy]ethyl-trimethyl-silane

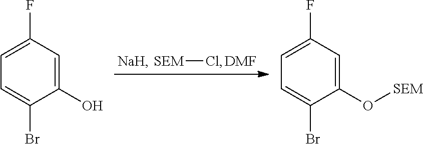

To a stirred solution of 2-bromo-5-fluoro-phenol (10 g, 52.3 mmol, 5.82 mL) in DMF (150 mL) was added NaH (4.19 g, 105 mmol, 60% in min. oil) at 0° C. After stirring for 0.5 h at 0° C., SEM-Cl (10.47 g, 62.8 mmol) was added. After an additional 1.5 h at rt, the mixture was quenched with saturated NH₄Cl (aq, 300 mL) and extracted with EtOAc (2×300 mL). The combined organic layers were washed with brine (300 mL), dried over anhydrous sodium sulfate and concentrated under reduced pressure. The crude residue purified by silica gel chromatography (petroleum ether-EtOAc, 10:1) to afford the title compound (16 g, 49.8 mmol, 95%) as a colorless oil.

### Step 2: trans-tert-Butyl N-[4-[4-fluoro-2-(2-trimethylsilylethoxymethoxy)anilino]-cyclohexyl]carbamate

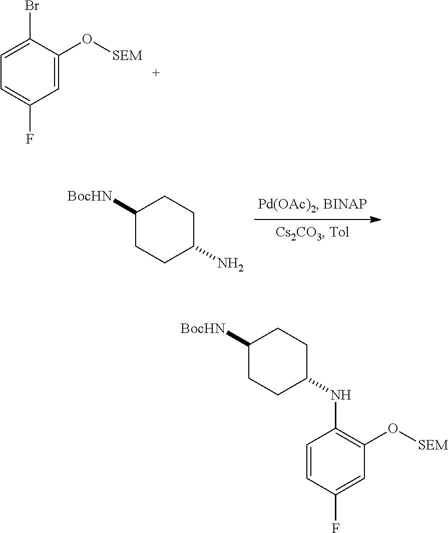 [SMILES: C[Si](C)(C)CCOCOc1cc(F)ccc1Br ; CC(C)(C)OC(=O)N[C@H]1CC[C@H](N)CC1 ; CC(C)(C)OC(=O)N[C@H]1CC[C@H](Nc2ccc(F)cc2OCOCC[Si](C)(C)C)CC1]

Prepared in a similar manner to Example 1U using trans-tert-butyl N-(4-aminocyclohexyl)carbamate (7.83 g, 36.5 mmol), 2-[(2-bromo-5-fluoro-phenoxy)methoxy]ethyl-trimethyl-silane (17.6 g, 54.8 mmol), BINAP (4.55 g, 7.30 mmol), Pd(OAc)₂ (1.65 g, 7.30 mmol) and Cs₂CO₃ (47.60 g, 146.1 mmol) and toluene (100 mL) to afford the title compound (4 g, 8.8 mmol, 24%) as a colorless oil.

LCMS (ES, m/z): 455.30 [M+H]⁺

### Step 3: trans-tert-Butyl N-[4-[N-cyclopropyl-4-fluoro-2-(2-trimethylsilylethoxymethoxy) anilino]cyclohexyl]carbamate

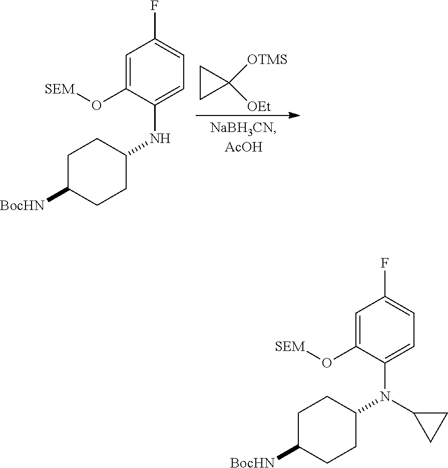

Prepared in a similar manner to Example 1U using trans-tert-butyl N-[4-[4-fluoro-2-(2-trimethylsilylethoxymethoxy)anilino]cyclohexyl]carbamate (2 g, 4.40 mmol), (1-ethoxycyclopropoxy)-trimethyl-silane (3.53 g, 20.2 mmol), NaBH₃CN (1.11 g, 17.6 mmol) and AcOH (40 mL) to afford the title compound (1.8 g, 3.64 mmol, 83%) as a yellow oil.

LCMS (ES, m/z): 495.35 [M+H]⁺

### Step 4: trans-tert-butyl N-[4-[N-Cyclopropyl-4-fluoro-2-(2-trimethylsilylethoxymethoxy) anilino]cyclohexyl]-N-methyl-carbamate

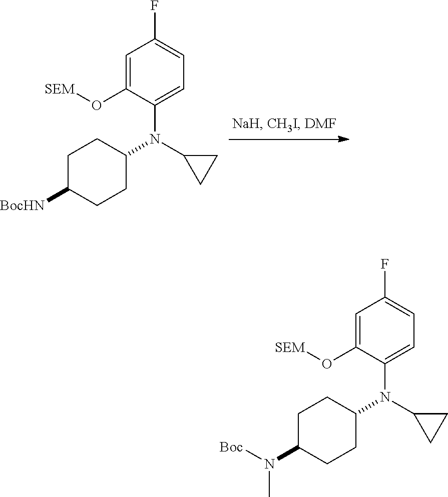

Prepared in a similar manner to Example 1U using trans-tert-butyl N-[4-[N-cyclopropyl-4-fluoro-2-(2-trimethylsilylethoxymethoxy)anilino]cyclohexyl]carbamate (1.8 g, 3.64 mmol), NaH (436 mg, 10.92 mmol, 60%), CH₃I (5.16 g, 36.38 mmol) and DMF (20 mL) to afford the title compound (1.3 g, 2.56 mmol, 70%) as a white solid.

LCMS (ES, m/z): 509.35 [M+H]⁺

### Step 5: trans-2-[Cyclopropyl-[4-(methylamino)cyclohexyl]amino]-5-fluoro-phenol

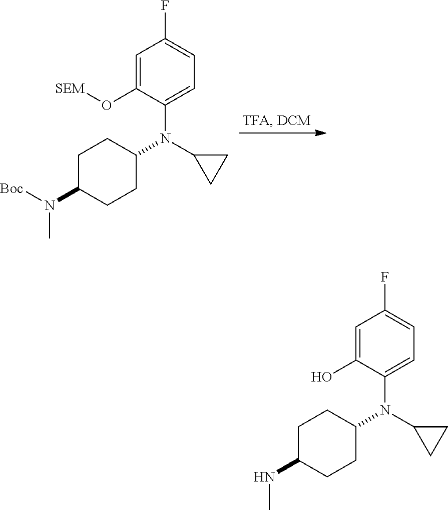

Prepared in a similar manner to Example 1U using trans-tert-butyl N-[4-[N-cyclopropyl-4-fluoro-2-(2-trimethylsilylethoxymethoxy)anilino]cyclohexyl]-N-methyl-carbamate (1.3 g, 2.56 mmol), DCM (10 mL) and TFA (3 mL) to afford the title compound (700 mg, assumed quantitative) as a yellow solid.

LCMS (ES, m/z): 279.15 [M+H]⁺

### Step 6: trans-8-[[4-(N-Cyclopropyl-4-fluoro-2-hydroxy-anilino)cyclohexyl]-methyl-amino]-5-methyl-6-oxo-1,5-naphthyridine-2-carbonitrile

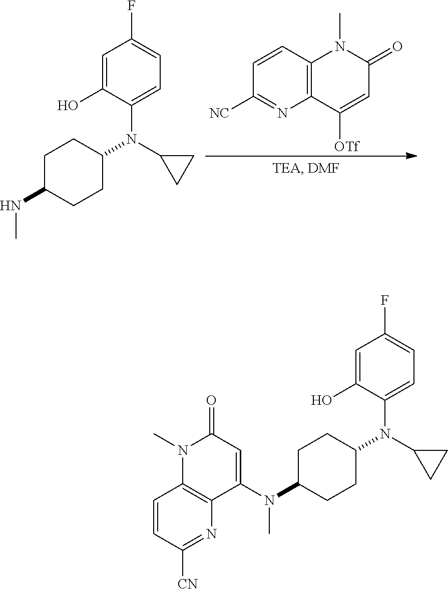

Prepared in a similar manner to Example 1U using trans-2-[cyclopropyl-[4-(methylamino)cyclohexyl]amino]-5-fluoro-phenol (100 mg, 359.24 μmol), (6-cyano-1-methyl-2-oxo-1,5-naphthyridin-4-yl) trifluoromethanesulfonate (119 mg, 359 μmol), TEA (109 mg, 1.08 mmol) and DMF (2 mL). The material was purified by HPLC (Column: YMC-Actus Triart C18, 30 mm×150 mm, Sum; Mobile Phase, A: water (10 mmol/L NH₄HCO₃) and B: ACN (52% to 72% in 9 min); Detector: 254 nm) to afford the title compound (29.2 mg, 63.3 μmol, 18%) as a light-yellow solid.

¹H NMR (400 MHz, DMSO-d₆) δ 8.66 (s, 1H), 8.14 (d, J=8.8 Hz, 1H), 8.03 (d, J=8.9 Hz, 1H), 7.15 (dd, J=8.3, 6.4 Hz, 1H), 6.60-6.52 (m, 2H), 5.86 (s, 1H), 4.26-4.17 (m, 1H), 3.51 (s, 3H), 3.03-2.96 (m, 1H), 2.80 (s, 3H), 1.96 (bd, J=12.6 Hz, 2H), 1.85 (bd, J=12.3 Hz, 2H), 1.65 (q, J=12.1 Hz, 2H), 1.41 (q, J=12.3 Hz, 2H), 0.48-0.43 (m, 2H), 0.10 (br s, 2H).

LCMS (ES, m/z): 462.25 [M+H]⁺

### Example 3Y: Synthesis of trans-8-[[4-(N-cyclopropylmethyl-N-4-fluoro-2-methoxyphenyl-anilino)cyclohexyl]-methyl-amino]-5-methyl-6-oxo-5,6-dihydro-1,5-naphthyridine-2-carbonitrile

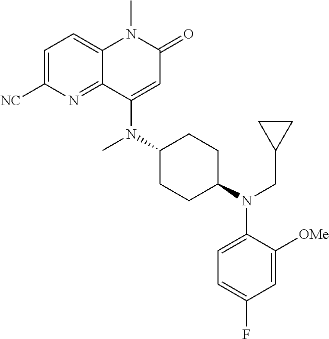 [SMILES: COc1cc(F)ccc1N(CC1CC1)[C@H]1CC[C@H](N(C)c2cc(=O)n(C)c3ccc(C#N)nc23)CC1]

### Step 1: cis trans-tert-Butyl-4-[(4-fluoro-2-methoxyphenyl-anilino)cyclohexyl]carbamate

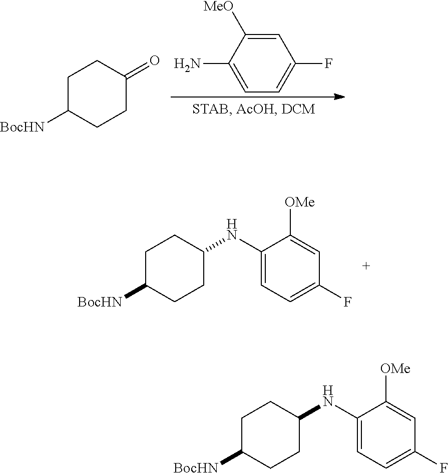

To a mixture of tert-butyl N-(4-oxocyclohexyl)carbamate (5.00 g, 23.4 mmol), 4-fluoro-2-methoxy-aniline (3.31 g, 2.76 mL, 23.4 mmol) in DCM (114.19 mL) was added acetic acid (268.4 μL, 4.69 mmol). After 15 min of stirring, sodium triacetoxyborohydride (9.32 g, 35.2 mmol, 80.0% purity) was added. The resulting mixture was stirred for 16 h, whereupon saturated NaHCO₃ (20 mL) and water (20 mL) were added. After 5 min, the layers were separated and the

organic layer was concentrated in vacuo. The crude residue was purified by silica gel chromatography (EtOAc-heptane, 5-20%), affording the cis-isomer (2.33 g, 29%) and trans-isomer (2.24 g, 28%) as white solids.

LCMS (ES, m/z): 339.45 [M+H]⁺.

### Step 2: tert-Butyl-4-[(N-cyclopropylmethyl-N-4-fluoro-2-methoxyphenyl-anilino) cyclohexyl] carbamate

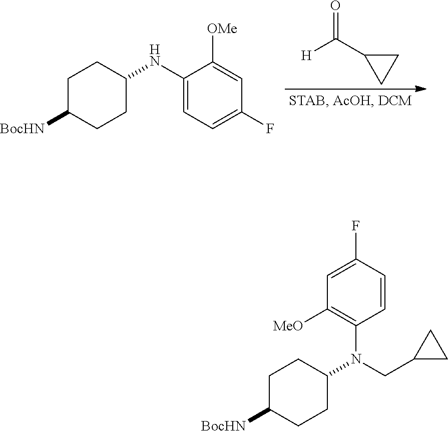 [SMILES: COc1cc(F)ccc1N[C@H]1CC[C@H](NC(=O)OC(C)(C)C)CC1 ; O=CC1CC1 ; COc1cc(F)ccc1N(CC1CC1)[C@H]1CC[C@H](NC(=O)OC(C)(C)C)CC1]

To a solution of trans-tert-butyl-4-[(4-fluoro-2-methoxyphenyl-anilino)cyclohexyl] carbamate (260 mg, 768 μmol) and cyclopropanecarbaldehyde (74.6 μL 999 μmol) in DCM (3.53 mL) was added acetic acid (88 μL, 1.54 mmol). After 15 min, sodium triacetoxyborohydride (244 mg, 1.15 mmol). After 16 h, additional cyclopropanecarbaldehyde (74.6 μL 999 μmol) and acetic acid (88 μL, 1.54 mmol) were added followed by sodium triacetoxyborohydride (244.25 mg, 1.15 mmol) 30 min later. After an additional 16 h, it was quenched with saturated NaHCO₃ and the layers were separated after bubbling subsided. The layers were separated and the aqueous layer extracted with DCM (2×). The combined organic layers were dried over anhydrous Na₂SO₄ and concentrated in vacuo. The crude residue was purified by silica gel chromatography (EtOAc-heptane, 0-30%), affording the title compound (111.5 mg, 37%).

LCMS (ES, m/z): 393.41 [M+H]⁺.

Steps 3-5: trans-8-[[4-(N-cyclopropylmethyl-N-4-fluoro-2-methoxyphenyl-anilino) cvclohexyl]-methyl-amino]-5-methyl-6-oxo-5,6-dihydro-1,5-naphthyridine-2-carbonitrile

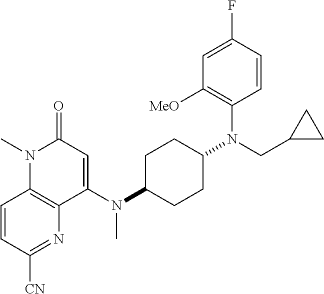 [SMILES: COc1cc(F)ccc1N(CC1CC1)[C@H]1CC[C@H](N(C)c2cc(=O)n(C)c3ccc(C#N)nc23)CC1]

Carried out in a similar manner to Step 3-5, Example 25. The crude material was purified by HPLC (Column: Sunfire OBD, C18, 19×250 mm, 5 μm; Mobile Phase, A: water with 0.1% formic acid and B: ACN with 0.1% formic acid, 10-40%), affording the title compound (6.4 mg, 13%) as an off-white solid.

¹H NMR (300 MHz, DMSO-d₆) δ ppm 8.19-8.11 (m, 1H), 8.07-8.01 (m, 1H), 7.10-7.02 (m, 1H), 6.82 (br d, J=10.5 Hz, 1H), 6.69-6.60 (m, 1H), 5.86 (s, 1H), 4.29-4.16 (m, 1H), 3.78 (s, 3H), 3.51 (s, 3H), 3.04-2.92 (m, 1H), 2.87 (br d, J=6.1 Hz, 2H), 2.81 (s, 3H), 2.73 (br d, J=1.47 Hz, 1H), 1.92-1.79 (m, 4H), 1.76-1.58 (m, 2H), 1.35-1.18 (m, 2H), 0.70-0.58 (m, 1H), 0.29-0.21 (m, 2H), 0.04-(−) 0.09 (m, 2H).

LCMS (ES, m/z): 490.48 [M+H]⁺.

### Example 3Z: Synthesis of cis-8-[[4-(N-cyclopropylmethyl-N-4-fluoro-2-methoxyphenyl-anilino) cyclohexyl]-methyl-amino]-5-methyl-6-oxo-5,6-dihydro-1,5-naphthyridine-2-carbonitrile

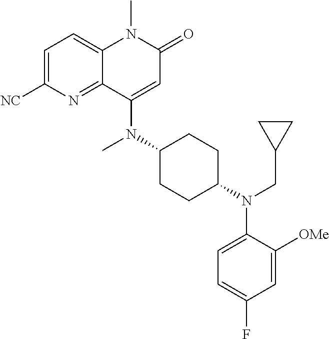 [SMILES: COc1cc(F)ccc1N(CC1CC1)[C@H]1CC[C@@H](N(C)c2cc(=O)n(C)c3ccc(C#N)nc23)CC1]

Prepared in a similar manner to example 45. The crude material was purified by silica gel chromatography (EtOAc-heptane, 0-100%), affording the title compound (17.3 mg, 12.8%) as an off-white solid.

¹H NMR (300 MHz, DMSO-d₆) δ ppm 8.13 (d, J=8.7 Hz, 1H), 8.03 (d, J=8.9H, 1H), 7.23 (t, J=7.6 Hz, 1H), 6.92-6.85 (m, 1H), 6.75-6.66 (m, 1H), 5.87 (s, 1H), 4.41-4.27 (m, 1H), 3.80 (s, 3H), 3.51 (s, 3H), 2.90 (s, 3H), 2.76 (d, J=6.7H, 2H), 2.09-1.91 (m, 2H), 1.83 (br d, J=13.9, 2H), 1.52-1.30 (m, 4H), 0.76-0.62 (m, 1H), 0.32-0.22 (m, 2H), −0.10-(−) 0.19 (m, 2H).

LCMS (ES, m/z): 490.54 [M+H]⁺.

### Example 3AA: Synthesis of trans-8-[[4-[Cyclopropylmethyl-(5-fluoro-2-pyridyl)aminol cyclohexyl]-methyl-amino]-5-methyl-6-oxo-1,5-naphthyridine-2-carbonitrile

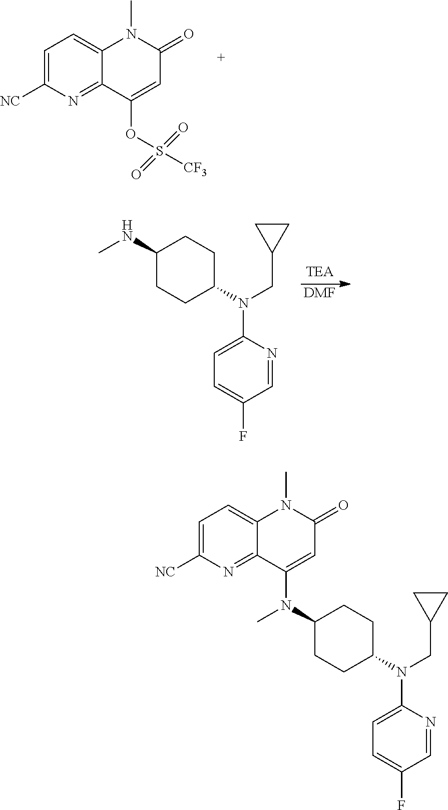

A mixture of trans-N⁴-(cyclopropylmethyl)-N⁴-(5-fluoro-2-pyridyl)-N¹-methyl-cyclohexane-1,4-diamine (250 mg, 511 μmol, 80%), (6-cyano-1-methyl-2-oxo-1,5-naphthyridin-4-yl) trifluoromethanesulfonate (170.3 mg, 511 μmol) and TEA (155 mg, 1.53 mmol) in DMF (2 mL) was stirred for 1 h at 80° C. After cooling rt, the solution was purified by reverse flash chromatography (Column, C18 silica gel, 80 g, 20-35 um; Mobile phase, A: water (0.5% TFA) and ACN (30-80% over 30 min); Detector: UV 254 nm), affording the title compound (69.4 mg, 140.9 μmol, 28%, 93.5% purity) as a yellow solid.

¹H NMR (300 MHz, DMSO-d₆) δ 8.16 (d, J=8.8 Hz, 1H), 8.08-8.02 (m, 2H), 7.46 (td, J=8.8, 3.2 Hz, 1H), 6.72 (dd, J=9.3, 3.0 Hz, 1H), 5.89 (s, 1H), 4.46-4.32 (m, 1H), 4.29-4.18 (m, H), 3.52 (s, 3H), 3.19 (d, J=6.0 Hz, 2H), 2.89 (s, 3H), 1.98-1.84 (m, 4H), 1.84-1.56 (m, 4H), 1.02-0.92 (m, 1H), 0.49-0.42 (m, 2H), 0.31-0.24 (m, 2H).

LCMS (ES, m/z): 461.15 [M+H]⁺

### Example 3BB: Synthesis of trans-8-((4-((4-fluoro-2-hydroxyphenyl)(oxetan-3-ylmethyl)amino) cyclohexyl)(methyl)amino)-5-methyl-6-oxo-5,6-dihydro-1,5-naphthyridine-2-carbonitrile

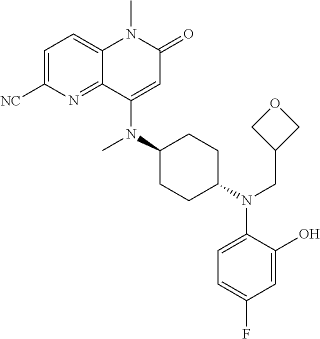 [SMILES: Cn1c(=O)cc(N(C)[C@H]2CC[C@H](N(CC3COC3)c3ccc(F)cc3O)CC2)c2nc(C#N)ccc21]

### Step 1: trans-tert-Butyl N-[4-[4-fluoro-N-(oxetan-3-ylmethyl)-2-(2-trimethylsilylethoxymethoxy) anilino]cyclohexyl]carbamate

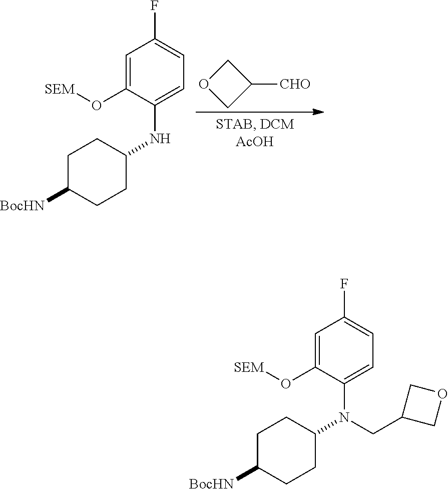

To a solution of trans-tert-butyl N-[4-[4-fluoro-2-(2-trimethylsilylethoxymethoxy)-anilino]cyclohexyl]carbamate (2.8 g, 6.16 mmol), oxetane-3-carbaldehyde (1.06 g, 12.32 mmol) in DCM (30 mL) was added AcOH (6 mL) followed by STAB (1.96 g, 9.24 mmol). After 1.5 h, the mixture was quenched with saturated NH₄Cl (aq, 30 mL) and extracted with DCM (3×30 mL). The combined organic layers were washed with brine (50 mL), dried over anhydrous sodium sulfate and concentrated under reduced pressure. The crude residue was purified by silica gel chromatography (petroleum ether-EtOAc, 2:1), affording the title compound (2.75 g, 5.24 mmol, 85%) as a yellow oil.

LCMS (ES, m/z): 525.35 [M+H]⁺

### Step 2: trans-tert-butyl N-[4-[4-Fluoro-N-(oxetan-3-ylmethyl)-2-(2-trimethylsilylethoxymethoxy) anilino]cyclohexyl]-N-methyl-carbamate

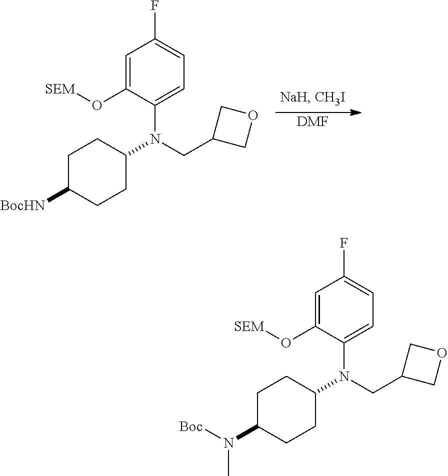

To a solution of trans-tert-butyl N-[4-[4-fluoro-N-(oxetan-3-ylmethyl)-2-(2-trimethylsilylethoxymethoxy)anilino]cyclohexyl]carbamate (2.75 g, 5.24 mmol) in DMF (25 mL) at 0° C. was added NaH (419 mg, 10.48 mmol, 60% in min. oil). After 30 min., CH₃I (2.98 g, 20.96 mmol) was added. After an additional 1.5 h at 0° C., the mixture was quenched with saturated NH₄Cl (aq, 50 mL) and extracted with EtOAc (3×50 mL). The combined organic layers were washed with brine (50 mL), dried over anhydrous sodium sulfate and concentrated under reduced pressure. The crude residue purified by silica gel chromatography (petroleum ether-EtOAc, 3:1) to afford the title compound (2.7 g, 5.01 mmol, 96%) as a yellow oil.

LCMS (ES, m/z): 539.45 [M+H]⁺

### Step 3: trans-N⁴-[4-Fluoro-2-(2-trimethylsilylethoxymethoxy)phenyl]-N¹-methyl-N⁴-(oxetan-3-ylmethyl)cyclohexane-1,4-diamine

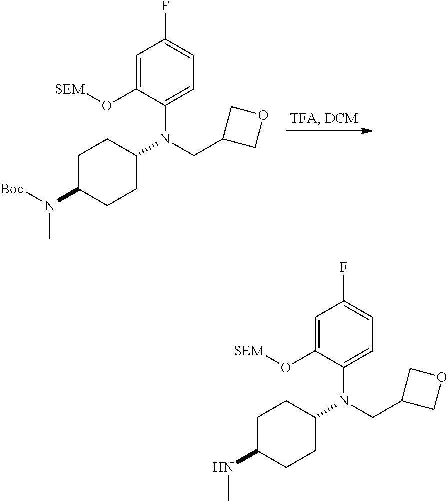

To a solution of trans-tert-butyl N-[4-[4-fluoro-N-(oxetan-3-ylmethyl)-2-(2-trimethylsilylethoxymethoxy)anilino]cyclohexyl]-N-methyl-carbamate (2.5 g, 4.64 mmol) in DCM (16 mL) was added TFA (4 mL). After 2 h, the resulting mixture was concentrated under reduced pressure, neutralized with saturated NaHCO₃ at 0° C. and extracted with EtOAc (3×50 mL). The combined organic layers were washed with brine (50 mL), dried over anhydrous sodium sulfate and concentrated under reduced pressure to afford the title compound (900 mg, 2.05 mmol, 44%) as a yellow oil.

LCMS (ES, m/z): 439.45 [M+H]⁺

### Step 4: trans-8-[[4-[4-Fluoro-N-(oxetan-3-ylmethyl)-2-(2-trimethylsilylethoxymethoxy) anilino]cyclohexyl]-methyl-amino]-5-methyl-6-oxo-1,5-naphthyridine-2-carbonitrile

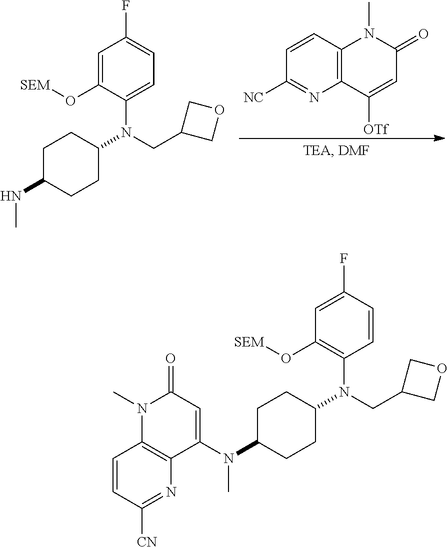

A solution of trans-N⁴-[4-fluoro-2-(2-trimethylsilylethoxymethoxy)phenyl]-N¹-methyl-N⁴-(oxetan-3-ylmethyl)cyclohexane-1,4-diamine (200 mg, 456 μmol), (6-cyano-1-methyl-2-oxo-1,5-naphthyridin-4-yl) trifluoromethanesulfonate (152 mg, 455.94 μmol) and TEA (92 mg, 911.88 μmol) in DMF (3 mL) was heated to 80° C. for 1 h, whereupon it was cooled to rt and purified by reverse flash chromatography (Column: C18 silica gel, 80 g, 20-35 um; Mobile Phase, A: water with 10 mmol/L NH₄HCO₃ and B: ACN, 5-60% over 40 min), affording the title compound (160 mg, 257 μmol, 56%) as a yellow oil.

LCMS (ES, m/z): 622.50 [M+H]⁺

### Step 5: trans-8-[[4-[4-Fluoro-2-hydroxy-N-(oxetan-3-ylmethyl)anilino]cyclohexyl]-methyl-amino]-5-methyl-6-oxo-1,5-naphthyridine-2-carbonitrile

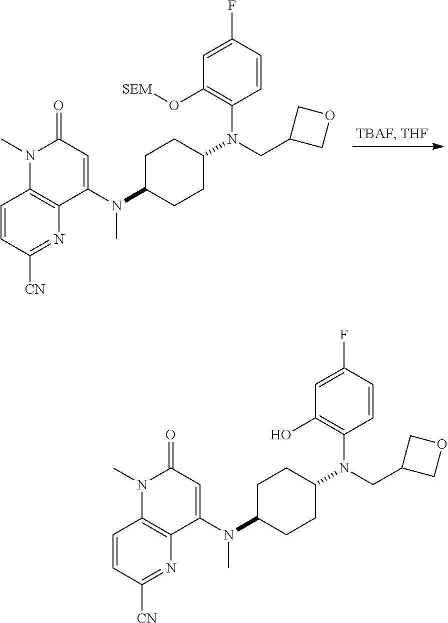

To a vial charged with trans-8-[[4-[4-fluoro-N-(oxetan-3-ylmethyl)-2-(2-trimethylsilylethoxymethoxy)anilino]cyclohexyl]-methyl-amino]-5-methyl-6-oxo-1,5-naphthyridine-2-carbonitrile (150 mg, 241 μmol) was added TBAF (1 M in THF, 2 mL). The solution was heated to 50° C. for 12 h. The mixture was cooled to room temperature, diluted with water (10 mL) and extracted with EtOAc (3×10 mL). The combined organic layers were washed with brine (10 mL), dried over anhydrous sodium sulfate and concentrated under reduced pressure. The crude residue was purified by reverse flash chromatography (Column: C18 silica gel, 40 g, 20-35 um; Mobile Phase, A: water with 10 mmol/L NH₄HCO₃ and B: ACN, 5-60% over 40 min), affording the title compound (37.5 mg, 69.6 μmol, 29%) as a light-yellow solid.

¹H NMR (400 MHz, DMSO-d₆) δ 8.68 (br s, 1H), 8.13 (d, J=8.8 Hz, 1H), 8.03 (d, J=8.9 Hz, 1H), 7.10 (dd, J=8.7, 6.4 Hz, 1H), 6.60 (dd, J=10.4, 2.8 Hz, 1H), 6.55 (td, J=8.8, 2.8 Hz, 1H) 5.87 (s, 1H), 4.38 (dd, J=7.6, 6.0 Hz, 2H), 4.22-4.12 (m, 1H), 4.12 (t, J=6.0 Hz, 2H), 3.51 (s, 3H), 3.25 (d, J=7.5 Hz, 2H), 2.80-2.67 (m, 5H), 1.94 (br d, J=11.6 Hz, 2H), 1.85 (br d, J=10.4 Hz, 2H), 1.67 (q, J=11.2 Hz, 2H), 1.32-1.20 (m, 2H).

LCMS (ES, m/z): 492.15 [M+H]⁺.

### Example 3CC: Synthesis of cis-6-Chloro-4-[[4-(N-cyclopropyl-2,4-difluoro-anilino)cyclohexyl]-methyl-amino]-1-methyl-2-oxo-1,5-naphthyridine-3-carbonitrile

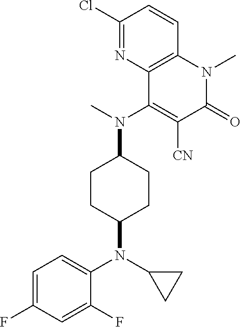 [SMILES: Cn1c(=O)c(C#N)c(N(C)[C@H]2CC[C@@H](N(c3ccc(F)cc3F)C3CC3)CC2)c2nc(Cl)ccc21]

### Step 1: cis-tert-Butyl N-[4-(2,4-difluoroanilino)cyclohexyl]carbamate

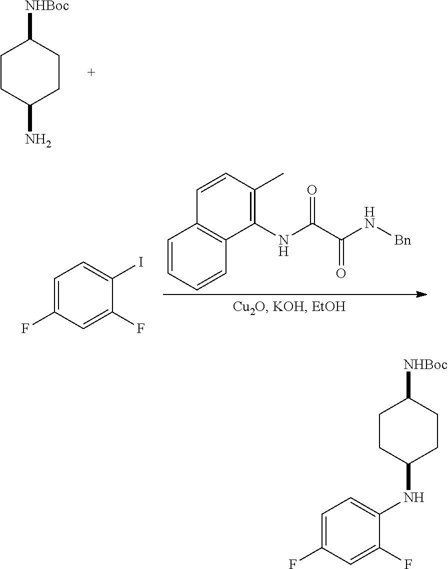

Performed in a similar manner as Step 1, Example 38 using cis-tert-butyl N-(4-aminocyclohexyl)carbamate (4.02 g, 18.7 mmol), 2,4-difluoro-1-iodo-benzene (3.0 g, 12.5 mmol), N¹-(2-methyl-1-naphthalenyl)-N²-(phenylmethyl)-ethanediamide (199 mg, 625 μmol), Cu₂O (89.4 mg, 625 μmol), KOH (911.8 mg, 16.25 mmol) and ethanol (25 mL). The crude residue was purified by silica gel chromatography (petroleum ether-EtOAc, 5:1), affording the title compound (380 mg, 1.16 mmol, 9.3%) as a white solid. LCMS (ES, m/z): 327.15 [M+H]⁺

### Steps 2-5: cis-6-Chloro-4-[[4-(N-cyclopropyl-2,4-difluoro-anilino)cyclohexyl]-methyl-amino]-1-methyl-2-oxo-1,5-naphthyridine-3-carbonitrile

Performed in an analogous manner as STEPS 3-6, Example 26. The crude material was purified by HPLC (Column: XBridge Prep OBD C18, 30×150 mm, 5 um; Mobile Phase, A: water with 50 mmol/L NH₄HCO₃ and B: ACN, 75-95% over 7 min), affording title compound (29.8 mg, 59.8 μmol, 9.9%) an off-white solid.

¹H NMR (400 MHz, DMSO-d₆) δ 8.04 (d, J=9.2 Hz, 1H), 7.78 (d, J=8.8 Hz, 1H), 7.38 (td, J=8.8, 6.4 Hz, 1H), 7.22 (ddd, J=9.1, 8.8, 2.8 Hz, 1H), 7.07-6.99 (m, 1H), 4.38-4.28 (m, 1H), 3.51 (s, 3H), 3.38-3.35 (m, 1H), 3.24 (s, 3H), 2.43-2.36 (m, 1H), 2.11-1.92 (m, 4H), 1.71-1.62 (m, 2H), 1.51 (t, J=13.2 Hz, 2H), 0.55-0.49 (m, 2H), 0.44-0.38 (m, 2H).

LCMS (ES, m/z): 498.15 [M+H]⁺

### Example 4

### Compounds of Structure (I-B-4)

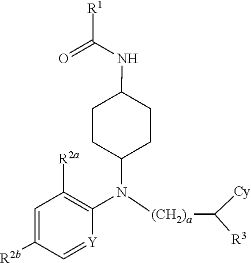

### Example 4A: Synthesis of cis-N-4-((cyclopropylmethyl)(phenyl)amino)cyclohexyl)-2-methyl-1H-indole-3-carboxamide

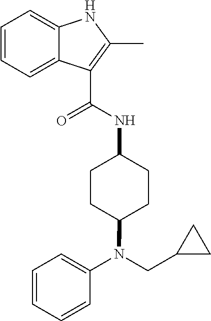 [SMILES: Cc1[nH]c2ccccc2c1C(=O)N[C@H]1CC[C@@H](N(CC2CC2)c2ccccc2)CC1]

### Step 1. cis-N¹-(cyclopropylmethyl)-N¹-phenylcyclohexane-1,4-diamine hydrochloride

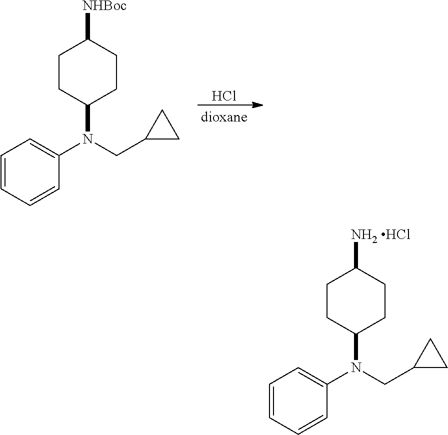

To a solution of cis-tert-butyl-4-((cyclopropylmethyl)(phenyl)amino)cyclohexyl) carbamate (18.9 mg, 54.9 μmol) in dioxane (200 μL) was added HCl (4 M in dioxane, 200 μL, 800 μmol). After stirring for 20 h, the suspension was concentrated under reduced pressure to afford the title compound as a white solid (assumed quantitative yield).

LCMS (ES, m/z): 245.10 [M−HCl+H]⁺

### Step 2. cis-N-4-((cyclopropylmethyl)(phenyl)amino)cyclohexyl)-2-methyl-1H-indole-3-carboxamide

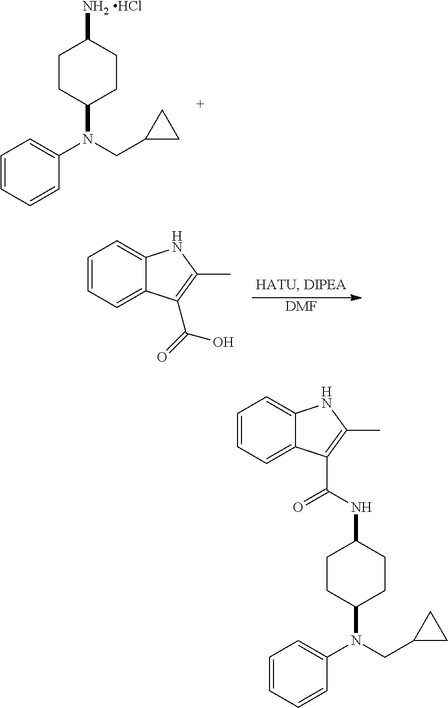

To a solution of 2-methyl-1H-indole-3-carboxylic acid (35.0 mg, 200 μmol) and HATU (76.5 mg, 200 μmol) in DMF (1.0 mL) at 0° C. was added DIPEA (35 μL, 200 μmol). After 30 min, half of the solution was removed and added to a solution of cis-N¹-(cyclopropylmethyl)-N¹-phenylcyclohexane-1,4-diamine hydrochloride (13.4 mg, 54.9 μmol) and DIPEA (38 μL, 219 μmol) in DMF (236 μL) was added. After 18 h, the crude material was purified by HPLC (Column: Xselect CSH OBD, C18, 19×150 mm, 5 μm; Mobile Phase, A: water with 0.05% formic acid and B: ACN with 0.05% formic acid, 5-40% over 15 min), affording the title compound (22 mg, 100%) as a white solid.

¹H NMR (300 MHz, DMSO-d₆) δ ppm 11.38 (s, 1H), 7.70 (br d, J=7.8 Hz, 1H), 7.41-7.28 (m, 2H), 7.18 (t, J=7.8 Hz, 2H), 7.12-7.00 (m, 2H), 6.86 (d, J=8.4 Hz, 2H), 6.62 (t, J=7.0, 1H), 4.13 (br s, 1H), 3.73-3.55 (m, 1H), 3.15 (d, J=5.5 Hz, 2H), 2.59 (s, 3H), 1.99 (br d, J=12.2 Hz, 2H), 1.91-1.54 (m, 6H), 1.04-0.91 (m, 1H), 0.55-0.45 (m, 2H), 0.29-0.23 (m, 2H).

LCMS (ES, m/z): 402.05 [M+H]⁺.

### Example 4B: Synthesis of trans-N-4-((cyclopropylmethyl)(phenyl)amino)cyclohexyl)-2-methyl-1H-indole-3-carboxamide

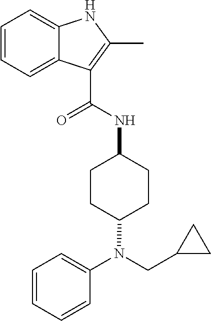 [SMILES: Cc1[nH]c2ccccc2c1C(=O)N[C@H]1CC[C@H](N(CC2CC2)c2ccccc2)CC1]

Prepared in an analogous manner to the examples as described herein. The crude material was purified by HPLC (Column: Xselect CSH OBD, C18, 19×150 mm, 5 μm; Mobile Phase, A: water with 0.05% formic acid and B: ACN with 0.05% formic acid, 5-40% over 15 min), affording the title compound (13.4 mg, 51%) as a white solid.

¹H NMR (300 MHz, DMSO-d₆) δ ppm 11.36 (s, 1H), 7.73-7.62 (m, 1H), 7.36-7.28 (m, 2H), 7.24-7.14 (m, 2H), 7.12-7.00 (m, 2H), 6.86 (d, J=8.4 Hz, 2H), 6.62 (t, J=7.0, 1H), 3.89-3.75 (m, 1H), 3.65-3.55 (m, 1H), 3.11 (d, J=5.0 Hz, 2H), 2.56 (s, 3H), 2.05-1.95 (m, 2H), 1.83-1.74 (m, 2H), 1.68-1.47 (m, 6H), 1.01-0.89 (m, 1H), 0.55-0.44 (m, 2H), 0.32-0.24 (m, 2H).

LCMS (ES, m/z): 402.25 [M+H]⁺.

### Example 4C: Synthesis of trans-2-Methyl-N-(4-((oxetan-3-ylmethyl)(phenyl)amino)cyclohexyl)-1H-indole-3-carboxamide

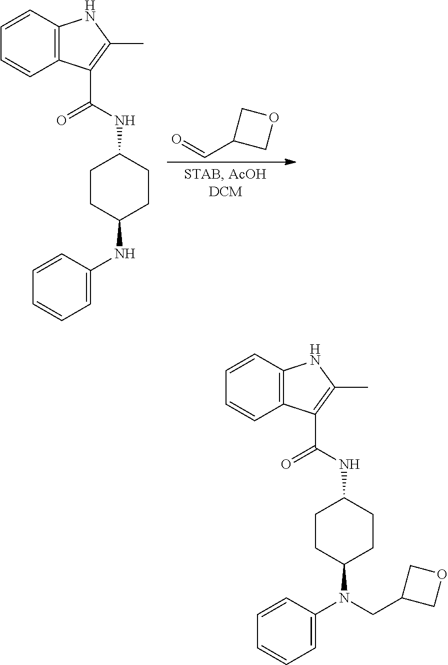

Prepared in a similar manner the examples described herein, using oxetane-3-carbaldehyde (61.9 mg, 720 μmol) to afford the title compound (14.5 mg, 25%) as a white solid.

¹H-NMR (DMSO-d₆, 300 MHz) δ (ppm): 11.36 (s, 1H), 7.72-7.65 (m, 1H), 7.36-7.27 (m, 1H), 7.18 (t, J=8.0 Hz, 3H), 7.11-7.00 (m, 2H), 6.79 (d, J=8.3 Hz, 2H), 6.69 (t, J=7.2 Hz, 1H), 4.58 (dd, J=7.8, 5.9 Hz, 2H), 4.35 (t, J=6.1 Hz, 2H), 3.92-3.75 (m, 1H), 3.56-3.45 (m, 3H), 3.21-3.05 (m, 2H), 2.56 (s, 3H), 2.00 (br d, J=11.3 Hz, 2H), 1.78-1.67 (m, 2H), 1.67-1.43 (m, 4H).

LCMS (ES, m/z): 417.93 [M+H]⁺.

### Example 4D: Synthesis of cis-2-Methyl-N-(4-((oxetan-3-ylmethyl)(phenyl)amino)cyclohexyl)-1H-indole-3-carboxamide

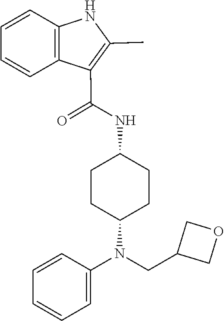 [SMILES: Cc1[nH]c2ccccc2c1C(=O)N[C@H]1CC[C@@H](N(CC2COC2)c2ccccc2)CC1]

### Step 1: cis-tert-Butyl (4-(phenylamino)cyclohexyl)carbamate

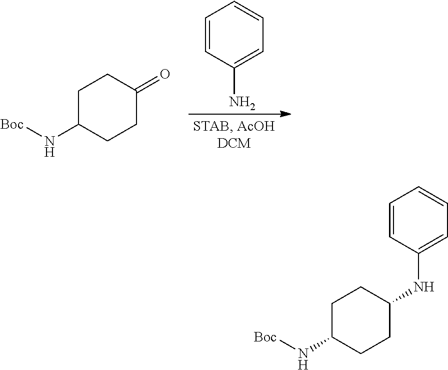

A solution of tert-butyl N-(4-oxocyclohexyl)carbamate (2.50 g, 11.7 mmol), aniline (2.18 g, 23.4 mmol, 2.14 mL), and acetic acid (1.41 g, 23.4 mmol, 1.34 mL) in DCM (34.7 mL) was stirred for 0.25 h at rt. To the mixture was added sodium triacetoxyborohydride (5.52 g, 23.4 mmol), and the suspension was stirred for 16 h. The mixture was quenched with saturated sodium hydrogen carbonate (30 mL) and the organic layer was concentrated under reduced pressure. The crude product was purified by silica gel chromatography with ethyl acetate/heptane (0-50%), affording the title compound (618 mg, 18%) as a white solid.

LCMS (ES, m/z): 290.82 [M+H]⁺.

### Step 2: cis-tert-Butyl (4-((oxetan-3-ylmethyl)(phenyl)amino)cyclohexyl)carbamate

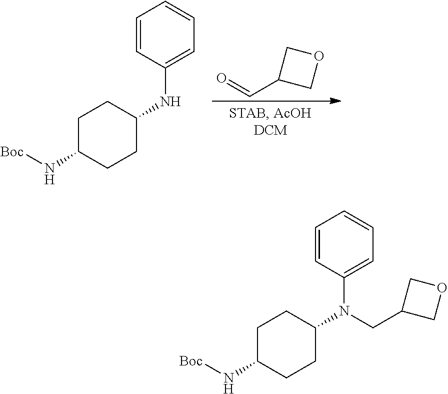

A solution of cis-tert-butyl (4-(phenylamino)cyclohexyl)carbamate (200 mg, 689 μmol), oxetane-3-carbaldehyde (119 mg, 1.38 mmol), and acetic acid (41.4 mg, 689 μmol, 39.4 μL) in DCM (2.72 mL) was stirred for 0.25 h at rt. To the mixture was added sodium triacetoxyborohydride (438 mg, 2.07 mmol), and the suspension was stirred for 16 h. The mixture was quench with saturated sodium hydrogen carbonate (2 mL) and the organic layer was concentrated under reduced pressure. The crude product was purified by silica gel chromatography with ethyl acetate/heptane (0-100%), affording the title compound (205 mg, 83%) as a white solid.

LCMS (ES, m/z): 360.71 [M+H]⁺.

### Step 3: cis-N-(Oxetan-3-ylmethyl)-N-phenylcyclohexane-1,4-diamine trifluoroacetate

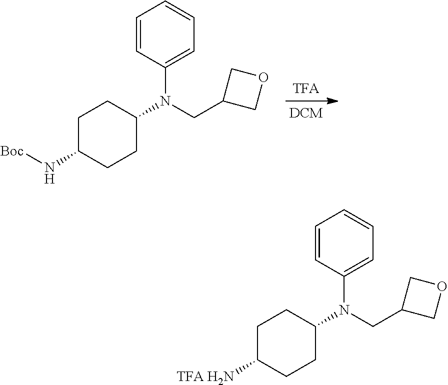

A solution of cis-tert-butyl (4-((oxetan-3-ylmethyl)(phenyl)amino)cyclohexyl)carbamate (160 mg, 444 μmol) and DCM (2.96 mL) was stirred for 0.25 h at 0° C. TFA (911 mg, 7.99 mmol, 615 μL) was added dropwise, and the mixture was stirred for 2 h at 0° C. Sodium hydrogen carbonate (746 mg, 8.88 mmol) and water (800 μL) were added, and the mixture was concentrated under reduced pressure. The crude product was purified by reversed phase chromatography (Column: SunFire Prep OBD C18, 19×250 mm, 5 μm; Mobile Phase, A: water (0.1% formic acid) and B: ACN (0.1% formic acid, 5-45% in 10 min); Detector: UV254/220 nm). The product fractions were lyophilized to afford the title compound (44.0 mg, 33%) as a white solid.

LCMS (ES, m/z): 260.91 [M+H]⁺.

### Step 4: cis-2-Methyl-N-(4-((oxetan-3-ylmethyl)(phenyl)amino)cyclohexyl)-1H-indole-3-carboxamide

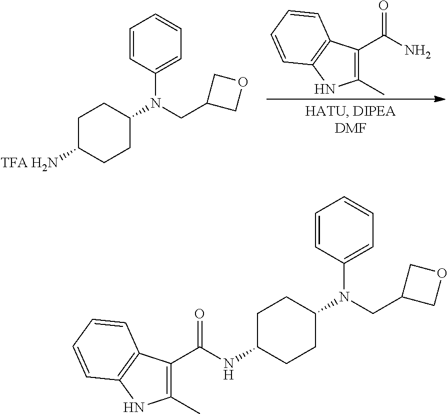

A solution of 2-methyl-1H-indole-3-carboxylic acid (7.99 mg, 45.6 μmol), HATU (17.4 mg, 45.6 μmol), and DIPEA (23.6 mg, 183 μmol, 31.8 μL) in DMF (212 μL) was stirred for 5 min at rt, and a solution of cis-N-(oxetan-3-ylmethyl)-N-phenylcyclohexane-1,4-diamine trifluoroacetate (22.0 mg, 45.63 μmol) in DMF (212 μL) was added. The mixture was stirred for 16 h at rt and purified by reversed phase chromatography (Column: SunFire Prep OBD C18, 19×250 mm, 5 μm; Mobile Phase, A: water (0.1% formic acid) and B: ACN (0.1% formic acid, 10-45% in 10 min); Detector: UV254/220 nm). The product fractions were lyophilized to afford the title compound (3.4 mg, 17%) as a white solid.

¹H-NMR (DMSO-d₆, 300 MHz) δ (ppm): 11.39 (s, 1H), 7.76-7.68 (m, 1H), 7.42 (br d, J=6.1 Hz, 1H), 7.38-7.29 (m, 1H), 7.19 (t, J=8.2 Hz, 2H), 7.13-7.03 (m, 2H), 6.83 (d, J=8.2 Hz, 2H), 6.71 (t, J=7.2 Hz, 1H), 4.59 (dd, J=7.8, 5.9 Hz, 2H), 4.34 (t, J=6.1 Hz, 2H), 4.11 (br s, 1H), 3.62-3.46 (m, 3H), 3.20-3.07 (m, 1H), 2.60 (s, 3H), 1.98 (br d, J=11.7 Hz, 2H), 1.92-1.78 (m, 2H), 1.74-1.60 (m, 2H), 1.54 (br d, J=10.6 Hz, 2H).

LCMS (ES, m/z): 418.02 [M+H]⁺.

### Example 4E: Synthesis of trans-2-Methyl-N-(4-(((3-methyloxetan-3-yl)methyl)(phenyl)amino) cyclohexyl)-1H-indole-3-carboxamide

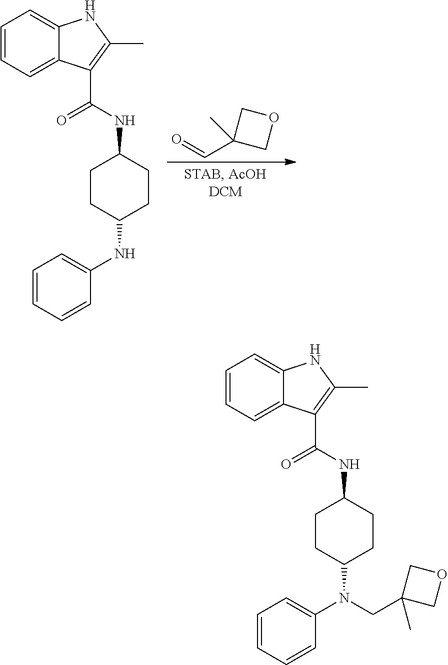

A solution of trans-2-methyl-N-(4-(phenylamino)cyclohexyl)-1H-indole-3-carboxamide (40.0 mg, 115 μmol), 3-methyloxetane-3-carbaldehyde (115 mg, 1.15 mmol), and acetic acid (6.9 mg, 115 μmol, 6.6 μL) in DCM (1.15 mL) was stirred for 0.25 h at rt. To the mixture was added sodium triacetoxyborohydride (97.6 mg, 460 μmol), and the suspension was stirred for 16 h. The mixture was extracted with saturated sodium hydrogen carbonate (2 mL) and concentrated under reduced pressure. The crude product was purified by reversed phase chromatography (Column: SunFire Prep OBD C18, 19×250 mm, 5 μm; Mobile Phase, A: water (0.1% formic acid) and B: ACN (0.1% formic acid, 5-50% in 10 min); Detector: UV254/220 nm). The product fractions were lyophilized to afford the title compound (3.9 mg, 7.7%) as a white solid.

¹H-NMR (DMSO-d₆, 300 MHz) δ (ppm): 11.35 (s, 1H), 7.72-7.65 (m, 1H), 7.33-7.28 (m, 1H), 7.25-7.16 (m, 3H), 7.11-7.00 (m, 2H), 6.85 (d, J=8.1 Hz, 2H), 6.78 (t, J=7.2 Hz, 1H), 4.41 (d, J=5.5 Hz, 2H), 3.97 (d, J=5.6 Hz, 2H), 3.24 (s, 2H), 2.55 (s, 3H), 1.98 (br d, J=7.7 Hz, 2H), 1.86-1.77 (m, 2H), 1.63-1.40 (m, 4H), 1.33 (s, 3H).

LCMS (ES, m/z): 432.62 [M+H]⁺.

### Example 4F: Synthesis of cis-N-(4-((Cyclopropylmethyl)(phenyl)amino)cyclohexyl)-1H-pyrrolo[3,2-b]pyridine-3-carboxamide

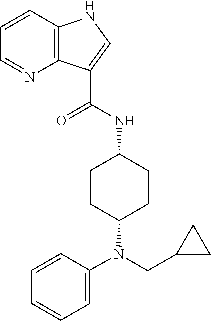 [SMILES: O=C(N[C@H]1CC[C@@H](N(CC2CC2)c2ccccc2)CC1)c1c[nH]c2cccnc12]

### Step 1: cis-N-(Cyclopropylmethyl)-N-phenylcyclohexane-1,4-diamine hydrochloride

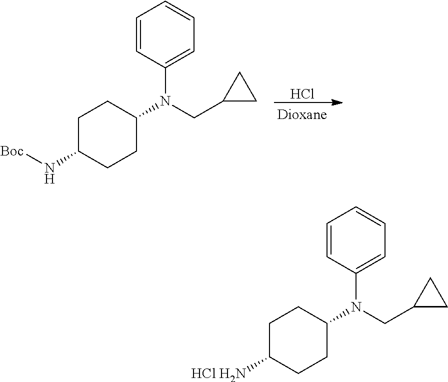

To a solution of cis-tert-butyl (4-((cyclopropylmethyl)(phenyl)amino)cyclohexyl) carbamate (70.0 mg, 203 μmol) and dioxane (1.52 mL) was added hydrogen chloride (4 M in dioxane, 1.02 mL). After stirring for 4 h at rt, the mixture was concentrated under reduced pressure, affording the title compound (assumed quantitative yield) as a white solid.

LCMS (ES, m/z): 245.51 [M+H]⁺.

### Step 2: cis-N-(4-((Cyclopropylmethyl)(phenyl)amino)cyclohexyl)-1H-pyrrolo[3,2-b]pyridine-3-carboxamide

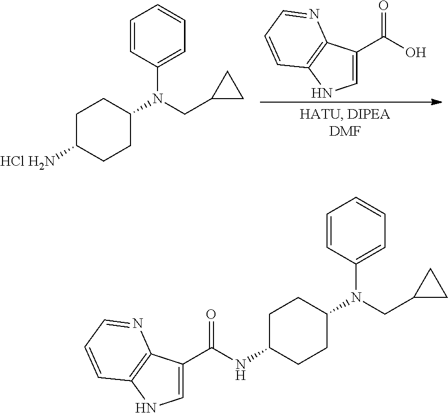

A solution of 1H-pyrrolo[3,2-b]pyridine-3-carboxylic acid (16.2 mg, 99.7 μmol), HATU (37.9 mg, 99.7 μmol), and DIPEA (64.4 mg, 499 μmol, 86.8 μL) in DMF (412 μL) was stirred for 5 min at 0° C. A solution of cis-N-(cyclopropylmethyl)-N-phenylcyclohexane-1,4-diamine hydrochloride (28.0 mg, 99.7 μmol) in DMF (412 μL) was added, and the mixture was stirred for 2 h at 0° C. The solution was purified by reversed phase chromatography (Column: SunFire Prep OBD C18, 19×250 mm, 5 μm; Mobile Phase, A: water (0.1% formic acid) and B: ACN (0.1% formic acid, 10-40% in 10 min); Detector: UV254/220 nm). The product fractions were lyophilized to afford the title compound (1.5 mg, 3.8%) as a white solid.

¹H-NMR (DMSO-d₆, 300 MHz) δ (ppm): 11.97 (br s, 1H), 9.32 (d, J=8.5 Hz, 1H), 8.46 (dd, J=4.7, 1.1 Hz, 1H), 8.18 (d, J=3.0 Hz, 1H), 7.96 (dd, J=8.2, 1.2 Hz, 1H), 7.31 (dd, J=8.2, 4.7 Hz, 1H), 7.18 (t, J=7.9 Hz, 2H), 6.87 (d, J=8.3 Hz, 2H), 6.61 (t, J=7.1 Hz, 1H), 4.34-4.24 (m, 1H), 3.74 (m, 1H), 3.22 (d, J=5.7 Hz, 2H), 1.93-1.63 (m, 8H), 1.07-0.92 (m, 1H), 0.56-0.44 (m, 2H), 0.27 (m, J=4.8 Hz, 2H).

LCMS (ES, m/z): 389.53 [M+H]⁺.

### Example 4G: Synthesis of cis-N-(4-((Cyclopropylmethyl)(phenyl)amino)cyclohexyl) pyrazolo[1,5-a]pyrimidine-3-carboxamide

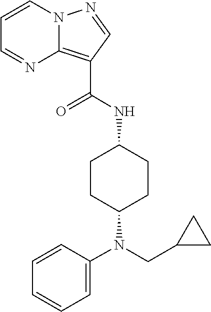 [SMILES: O=C(N[C@H]1CC[C@@H](N(CC2CC2)c2ccccc2)CC1)c1cnn2cccnc12]

Prepared in a similar manner to Example 4F, Step 2 using pyrazolo[1,5-a]pyrimidine-3-carboxylic acid (16.3 mg, 99.7 μmol) to afford the title compound (10.5 mg, 27%) as a white solid.

¹H-NMR (DMSO-d₆, 300 MHz) δ (ppm): 9.36 (d, J=7.0 Hz, 1H), 8.79 (d, J=3.9 Hz, 1H), 8.59 (s, 1H), 8.29 (br d, J=8.0 Hz, 1H), 7.33 (dd, J=7.0, 4.2 Hz, 1H), 7.18 (t, J=7.8 Hz, 2H), 6.87 (br d, J=8.3 Hz, 2H), 6.62 (t, J=7.2 Hz, 1H), 4.31-4.20 (m, 1H), 3.72 (br s, 1H), 3.17 (d, J=5.6 Hz, 2H), 1.97-1.64 (m, 8H), 1.06-0.90 (m, 1H), 0.45-0.56 (m, 2H), 0.27 (q, J=5.0 Hz, 2H).

LCMS (ES, m/z): 389.93 [M+H]⁺.

### Examples 5-214

Compounds of examples 5-214, as presented in Table 2, were prepared according to similar procedures as those described in Examples 1-4. LCMS in Table 2 is (ES, m/z) [M+H]⁺.

TABLE 2
Compounds 5-214

| Cmpd. | | | |
|---|---|---|---|
| No. | Structure | 1H NMR | LCMS |
| 5 | 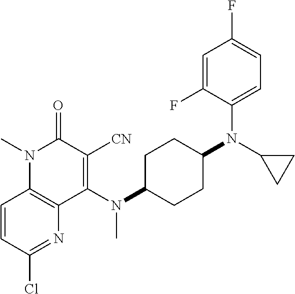 | (400 MHz, DMSO-d6) δ 8.05 (d, J = 9.0 Hz, 1H), 7.79 (d, J = 9.0 Hz, 1H), 7.42-7.34 (m, 1H), 7.25- 7.18 (m, 1H), 7.07-6.99 (m, 1H), 4.38-4.27 (m, 1H), 3.52 (s, 3H), 3.38-3.35 (m, 1H), 3.24 (s, 3H), 2.43-2.37 (m, 1H), 2.11-1.92 (m, 4H), 1.72-1.63 (m, 2H), 1.51 (t, J = 13.2 Hz, 2H), 0.55-0.49 (m, 2H), 0.43-0.38 (m, 2H) | 498.15 |
| 6 | 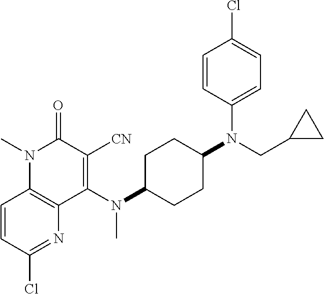 | (300 MHz, DMSO-d6) δ 8.09 (d, J = 8.9 Hz, 1H), 7.82 (d, J = 8.8 Hz, 1H), 7.25 (d, J = 8.6 Hz, 2H), 7.03 (d, J = 8.8 Hz, 2H), 4.45-4.31 (m, 1H), 3.64- 3.50 (m, 4H), 3.23 (s, 3H), 3.00 (d, J = 5.8 Hz, 2H), 2.18-1.99 (m, 2H), 1.86-1.60 (m, 4H), 1.56-1.40 (m, 2H), 0.84-0.69 (m, 1H), 0.35 (d, J = 7.5 Hz, 2H), 0.01 (d, J = 4.2 Hz, 2H) | 511.80 |
| 7 | 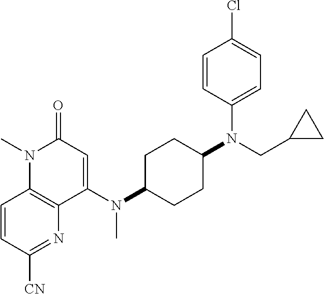 | (300 MHz, DMSO-d6) δ 8.15 (d, J = 9.0 Hz, 1H), 8.06 (d, J = 9.0 Hz, 1H), 7.28 (d, J = 8.1 Hz, 2H), 7.11 (d, J = 8.3 Hz, 2H), 5.99 (s, 1H), 4.31-4.16 (m, 1H), 3.60-3.48 (m, 4H), 3.01 (d, J = 6.1 Hz, 2H), 2.89 (s, 3H), 2.07-1.84 (m, 4H), 1.65-1.40 (m, 4H), 0.84-0.67 (m, 1H), 0.35 (d, J = 8.0 Hz, 2H), 0.00 (d, J = 4.8 Hz, 2H) | 476.84 |
| 8 | 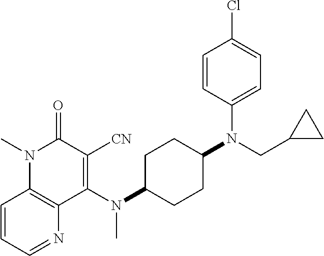 | (300 MHz, DMSO-d6) δ 8.58 (d, J = 4.1 Hz, 1H), 8.03 (d, J = 8.6 Hz, 1H), 7.72 (dd, J = 4.0, 8.5 Hz, 1H), 7.21 (d, J = 8.5 Hz, 2H), 6.96 (d, J = 8.6 Hz, 2H), 4.48 (br s, 1H), 3.57 (s, 4H), 3.23 (s, 3H), 3.00 (d, J = 5.7 Hz, 2H), 2.16-1.98 (m, 2H), 1.82- 1.59 (m, 4H), 1.46 (d, J = 9.8 Hz, 2H), 0.89-0.74 (m, 1H), 0.37 (d, J = 7.9 Hz, 2H), 0.06 (d, J = 4.7 Hz, 2H) | 476.58 |
| 9 | 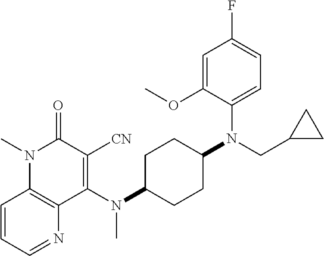 | (300 MHz, DMSO-d6) δ 8.52 (d, J = 4.1 Hz, 1H), 7.97 (d, J = 8.5 Hz, 1H), 7.68 (dd, J = 4.1, 8.8 Hz, 1H), 7.23 (t, J = 7.5 Hz, 1H), 6.90 (d, J = 10.0 Hz, 1H), 6.71 (t, J = 8.1 Hz, 1H), 4.52-4.34 (m, 1H), 3.80 (s, 3H), 3.55-3.50 (m, 3H), 3.47 (br s, 1H), 3.30 (s, 3H), 2.76 (d, J = 6.6 Hz, 2H), 2.23-2.04 (m, 2H), 1.83 (d, J = 13.1 Hz, 2H), 1.57 (d, J = 9.8 Hz, 2H), 1.39 (t, J = 12.0 Hz, 2H), 0.77-0.62 (m, 1H), 0.27 (d, J = 7.9 Hz, 2H), −0.14 (d, J = 4.8 Hz, 2H) | 490.96 |
| 10 | 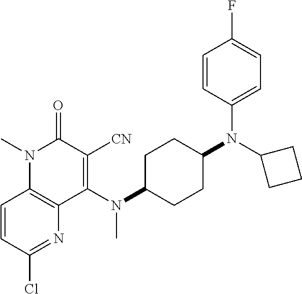 | (400 MHz, Chloroform-d) δ 7.60 (d, J = 8.8, 1H), 7.47 (d, J = 8.9, 1H), 7.12-7.05 (m, 2H), 7.00 (t, J = 8.7, 2H), 4.46 (t, J = 11.9 Hz, 1H), 3.68-3.57 (m, 4H), 3.40 (s, 3H), 3.13 (s, 1H), 2.20 (q, J = 12.2 Hz, 2H), 1.96-1.80 (m, 4H), 1.77-1.58 (m, 4H), 1.52-1.38 (m, 4H) | 494.20 |
| 11 | 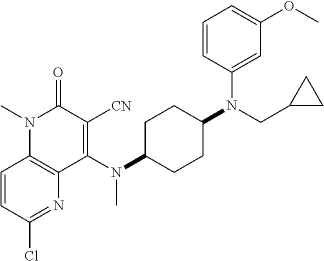 | (300 MHz, DMSO-d6) δ 8.09 (d, J = 9.0 Hz, 1H), 7.81 (d, J = 8.8 Hz, 1H), 7.13 (t, J = 8.2 Hz, 1H), 6.62 (d, J = 8.2 Hz, 1H), 6.53 (s, 1H), 6.42 (d, J = 7.9 Hz, 1H), 4.44-4.32 (m, 1H), 3.72 (s, 3H), 3.63- 3.50 (m, 4H), 3.23 (s, 3H), 2.99 (d, J = 5.8 Hz, 2H), 2.17-2.03 (m, 2H), 1.88-1.64 (m, 4H), 1.56-1.41 (m, 2H), 0.86-0.71 (m, 1H), 0.35 (d, J = 7.9 Hz, 2H), 0.03 (d, J = 4.7 Hz, 2H) | 507.33 |
| 12 | 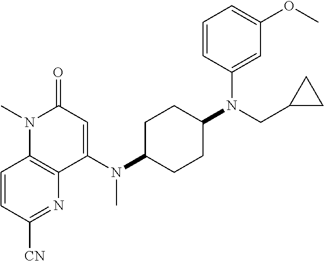 | (300 MHz, DMSO-d6) δ 8.16 (d, J = 9.0 Hz, 1H), 8.06 (d, J = 9.0 Hz, 1H), 7.44-7.28 (m, 1H), 7.23- 7.10 (m, 1H), 6.80-6.43 (m, 2H), 5.98 (s, 1H), 4.36-4.15 (m, 1H), 3.90-3.69 (m, 4H), 3.55-3.49 (m, 4H), 3.12-2.83 (m, 4H), 2.25-1.74 (m, 4H), 1.73-1.37 (m, 4H), 0.88-0.64 (m, 1H), 0.42-0.26 (m, 2H), 0.13-−0.07 (m, 2H) | 472.48 |
| 13 | 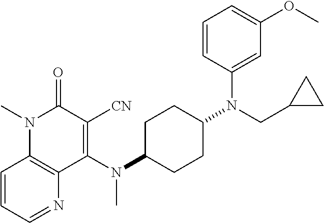 | (300 MHz, DMSO-d6) δ 8.57 (d, J = 3.8 Hz, 1H), 7.98 (d, J = 8.6 Hz, 1H), 7.70 (dd, J = 4.2, 8.7 Hz, 1H), 7.08 (t, J = 8.2 Hz, 1H), 6.46 (d, J = 8.5 Hz, 1H), 6.35 (s, 1H), 6.23 (d, J = 7.5 Hz, 1H), 4.49- 4.33 (m, 1H), 3.80-3.63 (m, 4H), 3.53 (s, 3H), 3.29 (s, 3H), 3.03 (d, J = 5.4 Hz, 2H), 2.15-1.95 (m, 4H), 1.82 (d, J = 11.1 Hz, 2H), 1.68-1.47 (m, 2H), 1.01-0.82 (m, 1H), 0.47 (d, J = 7.8 Hz, 2H), 0.23 (d, J = 4.5 Hz, 2H) | 472.85 |
| 14 | 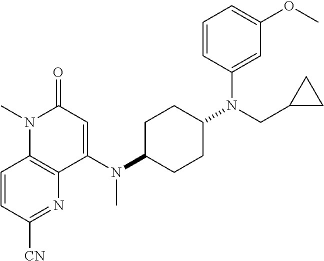 | 1H NMR (300 MHz, DMSO-d6) δ = 8.15 (d, J = 8.8 Hz, 1H), 8.04 (d, J = 8.8 Hz, 1H), 7.08 (t, J = 8.2 Hz, 1H), 6.45 (d, J = 8.4 Hz, 1H), 6.34 (s, 1H), 6.22 (d, J = 8.0 Hz, 1H), 5.89 (s, 1H), 4.45-4.28 (m, 1H), 3.77-3.57 (m, 4H), 3.52 (s, 3H), 3.03 (d, J = 5.2 Hz, 2H), 2.51 (s, 3H), 2.04-1.73 (m, 6H), 1.73-1.45 (m, 2H), 0.99-0.82 (m, 1H), 0.48 (d, J = 7.8 Hz, 2H), 0.24 (d, J = 4.6 Hz, 2H) | 472.24 |
| 15 | 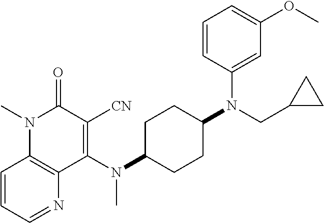 | (300 MHz, DMSO-d6) δ 8.58 (d, J = 3.9 Hz, 1H), 8.03 (d, J = 8.7 Hz, 1H), 7.72 (dd, J = 4.1, 8.5 Hz, 1H), 7.10 (t, J = 8.1 Hz, 1H), 6.56 (d, J = 8.3 Hz, 1H), 6.46 (s, 1H), 6.35 (d, J = 7.9 Hz, 1H), 4.48 (br s, 1H), 3.71 (s, 3H), 3.64-3.51 (m, 4H), 3.23 (s, 3H), 2.99 (d, J = 5.6 Hz, 2H), 2.17-1.99 (m, 2H), 1.82-1.59 (m, 4H), 1.52-1.37 (m, 2H), 0.89-0.73 (m, 1H), 0.37 (d, J = 7.8 Hz, 2H), 0.06 (d, J = 4.6 Hz, 2H) | 472.41 |
| 16 | 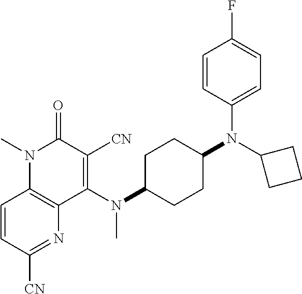 | (300 MHz, DMSO-d6) δ 8.25 (d, J = 8.8 Hz, 1H), 8.13 (d, J = 8.9 Hz, 1H), 7.25-7.10 (m, 4H), 4.42- 4.29 (m, 1H), 3.75-3.63 (m, 1H), 3.53 (s, 3H), 3.29 (s, 3H), 3.13 (s, 1H), 2.16 (q, J = 11.7 Hz, 2H), 1.93-1.73 (m, 4H), 1.68-1.57 (m, 4H), 1.55-1.36 (m, 4H) | 485.15 |
| 17 | 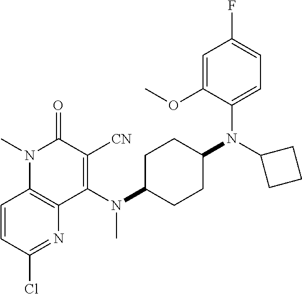 | (400 MHz, Chloroform-d) δ 7.59 (d, J = 8.9 Hz, 1H), 7.47 (d, J = 8.9 Hz, 1H), 7.1-7.02 (m, 1H), 6.63-6.56 (m, 2H), 4.50-4.41 (m, 1H), 3.77 (s, 3H), 3.75-3.64 (m, 1H), 3.60 (s, 3H), 3.41 (s, 3H), 3.39- 3.33 (m, 1H), 2.22 (q, J = 11.8 Hz, 2H), 1.86 (br s, 4H), 1.68 (br d, J = 10.2 Hz, 4H), 1.51-1.37 (m, 4H) | 524.15 |
| 18 | 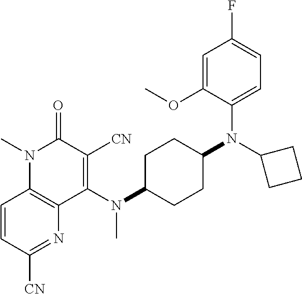 | (300 MHz, Chloroform-d) δ 7.82 (d, J = 8.8 Hz, 1H), 7.69 (d, J = 8.8 Hz, 1H), 7.10-7.03 (m, 1H), 6.65-6.54 (m, 2H), 4.53-4.41 (m, 1H), 3.78 (s, 3H), 3.73-3.70 (m, 1H), 3.62 (s, 3H), 3.45 (s, 3H), 3.38 (s, 1H), 2.25 (q, J = 11.9 Hz, 2H), 1.98-1.79 (m, 4H), 1.73-1.61 (m, 4H), 1.51-1.42 (m, 4H) | 515.25 |
| 19 | 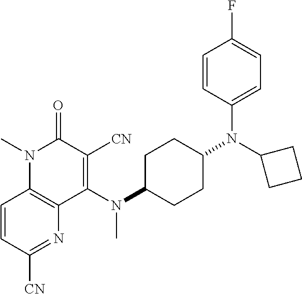 | (300 MHz, DMSO-d6) δ 8.22 (d, J = 8.9 Hz, 1H), 8.10 (d, J = 9.0 Hz, 1H), 7.08-6.91 (m, 4H), 4.25- 4.11 (m, 1H), 3.98-3.86 (m, 1H), 3.51 (s, 3H), 3.22 (s, 3H), 2.92 (t, J = 11.9 Hz, 1H), 2.12-1.78 (m, 8H), 1.74-1.54 (m, 4H), 1.35-1.18 (m, 2H) | 485.25 |
| 20 | 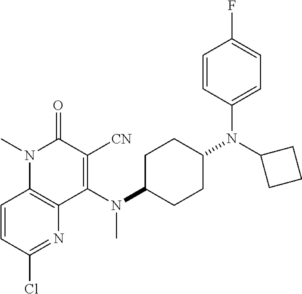 | (300 MHz, DMSO-d6) δ 8.01 (d, J = 9.0 Hz, 1H), 7.75 (d, J = 9.0 Hz, 1H), 7.20-6.82 (m, 4H), 4.21- 4.07 (m, 1H), 3.98-3.85 (m, 1H), 3.49 (s, 3H), 3.19 (s, 3H), 2.98-2.84 (m, 1H), 2.11-1.79 (m, 8H), 1.74-1.51 (m, 4H), 1.36-1.16 (m, 2H) | 494.20 |
| 21 | 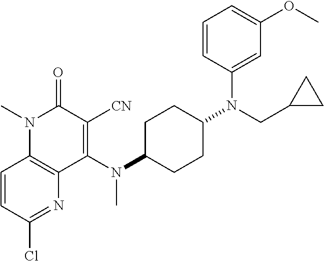 | 1H NMR (300 MHz, DMSO-d6) δ = 8.06 (d, J = 9.0 Hz, 1H), 7.81 (d, J = 8.8 Hz, 1H), 7.08 (t, J = 8.2 Hz, 1H), 6.46 (d, J = 8.4 Hz, 1H), 6.35 (s,1H), 6.23 (d, J = 8.2 Hz, 1H), 4.42-4.29 (m, 1H), 3.73- 3.63 (m, 4H), 3.53 (s, 3H), 3.27 (s, 3H), 3.03 (d, J = 5.4 Hz, 2H), 2.11-2.00 (m, 4H), 1.83 (d, J = 10.7 Hz, 2H), 1.71-1.52 (m, 2H), 0.97-0.86 (m, 1H), 0.54-0.43 (m, 2H), 0.27-0.19 (m, 2H) | 507.38 |
| 22 | 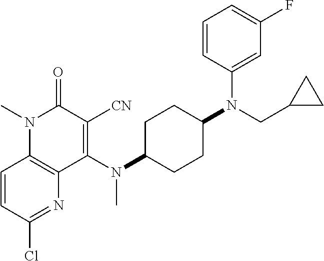 | 1H NMR (300 MHz, DMSO-d6) δ = 8.11 (d, J = 8.6 Hz, 1H), 7.83 (d, J = 9.3 Hz, 1H), 7.20 (q, J = 7.7 Hz, 1H), 6.80-6.66 (m, 2H), 6.50 (t, J = 8.3 Hz, 1H), 4.43-4.35 (m, 1H), 3.73-3.63 (m, 1H), 3.56 (s, 4H), 3.22 (s, 3H), 3.06 (d, J = 5.5 Hz, 2H), 2.16- 2.01 (m, 2H), 1.79-1.64 (m, 4H), 1.55-1.44 (m, 2H), 0.83 (m, 1H), 0.38 (d, J = 8.0 Hz, 2H), 0.07 (d, J = 4.5 Hz, 2H) | 497.4 |
| 23 | 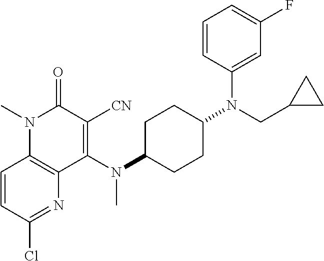 | 1H NMR (300 MHz, DMSO-d6) δ = 8.06 (d, J = 8.9 Hz, 1H), 7.81 (d, J = 8.9 Hz, 1H), 7.18 (q, J = 7.9 Hz, 1H), 6.72-6.55 (m, 2H), 6.38 (t, J = 8.3 Hz, 1H), 4.44-4.29 (m, 1H), 3.80-3.66 (m, J = 10.6, 10.6 Hz, 1H), 3.53 (s, 3H), 3.06 (d, J = 5.2 Hz, 2H), 2.16-1.98 (m, 4H), 1.82 (d, J = 11.3 Hz, 2H), 1.64 (d, J = 6.0 Hz, 2H), 0.92 (m, 1H), 0.49 (d, J = 7.9 Hz, 2H), 0.25 (d, J = 4.7 Hz, 2H) | 497.4 |
| 24 | 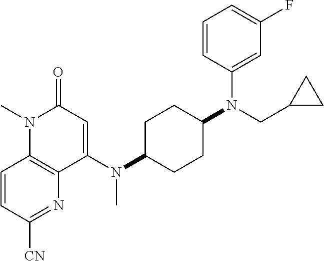 | (300 MHz, DMSO-d6) δ 8.17 (d, J = 8.7 Hz, 1H), 8.07 (d, J = 9.0 Hz, 1H), 7.23 (q, J = 7.6 Hz, 1H), 6.87-6.75 (m, 2H), 6.63-6.53 (m, 1H), 6.06 (s, 1H), 4.16 (br s, 1H), 3.70-3.60 (m, 2H), 3.54 (s, 3H), 3.09 (d, J = 5.9 Hz, 2H), 2.91 (s, 3H), 2.09-1.83 (m, 4H), 1.72-1.44 (m, 4H), 0.90-0.72 (m, 1H), 0.44-0.33 (m, 2H), 0.12-0.02 (m, 2H) | 460.54 |
| 25 | 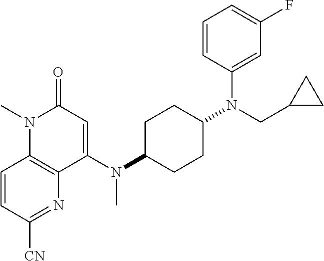 | (300 MHz, DMSO-d6) δ 8.16 (d, J = 8.7 Hz, 1H), 8.05 (d, J = 9.3 Hz, 1H), 7.17 (q, J = 8.1 Hz, 1H), 6.73-6.51 (m, 2H), 6.37 (t, J = 8.1 Hz, 1H), 5.89 (s, 1H), 4.48-4.27 (m, 1H), 3.71 (t, J = 11.0 Hz, 1H), 3.52 (s, 3H), 3.07 (d, J = 5.4 Hz, 2H), 2.90 (s, 3H), 2.03-1.88 (m, 4H), 1.88-1.72 (m, 2H), 1.72-1.51 (m, 2H), 0.97-0.84 (m, 1H), 0.49 (d, J = 7.7 Hz, 2H), 0.26 (d, J = 4.7 Hz, 2H) | 460.99 |
| 26 | 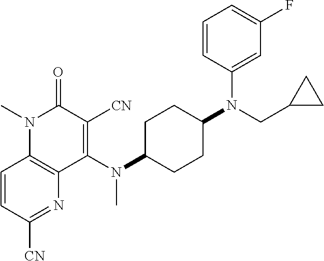 | (300 MHz, DMSO-d6) δ 8.29 (d, J = 8.8 Hz, 1H), 8.19 (d, J = 8.8 Hz, 1H), 7.21 (q, J = 7.6 Hz, 1H), 6.84-6.66 (m, 2H), 6.53 (t, J = 8.3 Hz, 1H), 4.54- 4.35 (m, 1H), 3.98-3.62 (m, 2H), 3.61-3.44 (m, 3H), 3.26 (s, 3H), 3.06 (d, J = 5.7 Hz, 2H), 2.18- 2.03 (m, 2H), 1.84-1.65 (m, 4H), 1.62-1.38 (m, 2H), 0.99-0.76 (m, 1H), 0.38 (d, J = 7.8 Hz, 2H), 0.07 (d, J = 4.4 Hz, 2H) | 486.25 |
| 27 | 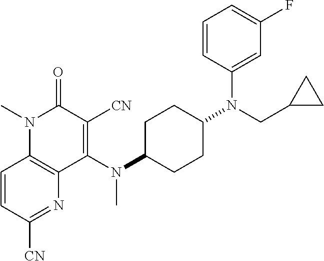 | (300 MHz, DMSO-d6) δ = 8.27 (d, J = 8.8 Hz, 1H), 8.14 (d, J = 8.9 Hz, 1H), 7.18 (q, J = 7.9 Hz, 1H), 6.76-6.53 (m, 2H), 6.38 (t, J = 8.2 Hz, 1H), 4.50-4.32 (m, 1H), 3.74 (t, J = 10.7 Hz, 2H), 3.54 (s, 3H), 3.30 (s, 3H), 3.07 (d, J = 5.3 Hz, 2H), 2.19- 1.96 (m, 4H), 1.83 (d, J = 11.5 Hz, 2H), 1.75-1.55 (m, 2H), 0.98-0.85 (m, 1H), 0.49 (d, J = 8.0 Hz, 2H), 0.26 (d, J = 4.9 Hz, 2H) | 485.95 |
| 28 | 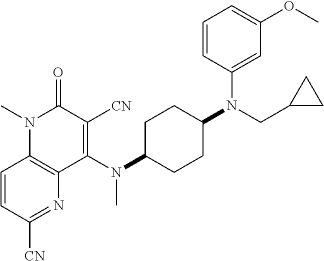 | (300 MHz, DMSO-d6) δ = 8.27 (d, J = 8.4 Hz, 1H), 8.17 (d, J = 9.3 Hz, 1H), 7.14 (t, J = 8.0 Hz, 1H), 6.65 (d, J = 8.1 Hz, 1H), 6.59-6.53 (m, 1H), 6.44 (d, J = 8.3 Hz, 1H), 4.46-4.35 (m, 1H), 3.72 (s, 3H), 3.61-3.49 (m, 4H), 3.27 (s, 3H), 2.99 (d, J = 5.9 Hz, 2H), 2.29-2.00 (m, 2H), 1.90-1.64 (m, 4H), 1.59-1.41 (m, 2H), 0.85-0.72 (m, 1H), 0.35 (d, J = 8.0 Hz, 2H), 0.02 (d, J = 4.7 Hz, 2H) | 497.45 |
| 29 | 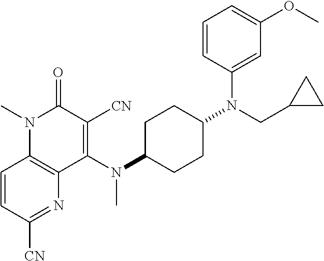 | (300 MHz, DMSO-d6) δ = 8.27 (d, J = 8.9 Hz, 1H), 8.14 (d, J = 8.9 Hz, 1H), 7.08 (t, J = 8.2 Hz, 1H), 6.46 (d, J = 8.4 Hz, 1H), 6.35 (s, 1H), 6.23 (d, J = 8.0 Hz, 1H), 4.49-4.29 (m, 1H), 3.79-3.63 (m, 4H), 3.58-3.43 (m, 4H), 3.30 (s, 3H), 3.03 (d, J = 5.3 Hz, 2H), 2.17-1.96 (m, 4H), 1.84 (d, J = 10.9 Hz, 2H), 1.72-1.53 (m, 2H), 0.98-0.85 (m, 1H), 0.54-0.42 (m, 2H), 0.30-0.19 (m, 2H) | 498.30 |
| 30 | 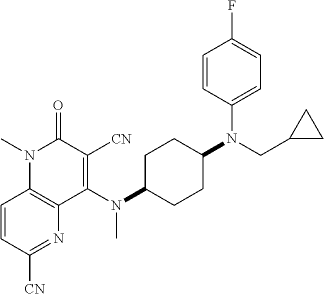 | (400 MHz, DMSO-d6) δ 8.25 (d, J = 8.8 Hz, 1H), 8.13 (d, J = 8.9 Hz, 1H), 7.24-7.15 (m, 2H), 7.15- 7.06 (m, 2H), 4.41-4.36 (m, 1H), 3.53 (s, 3H), 3.39-3.45 (m, 1H), 3.28 (s, 3H), 2.88 (d, J = 6.5 Hz, 2H), 2.12 (q, J = 10.7, 2H), 1.86 (br d, J = 13.7 Hz, 2H), 1.65 (br d, J = 11.9 Hz, 2H), 1.46 (t, J = 12.8 Hz, 2H), 0.74-0.64 (m, 1H), 0.35-0.26 (m, 2H), −0.04-−0.08 (m, 2H) | 485.30 |
| 31 | 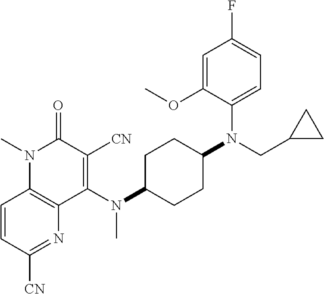 | (300 MHz, DMSO-d6) δ 8.24 (d, J = 8.8 Hz, 1H), 8.12 (d, J = 8.8 Hz, 1H), 7.25 (t, J = 7.6 Hz, 1H), 6.89 (d, J = 10.9 Hz, 1H), 6.71 (br t, J = 7.8 Hz, 1H), 4.44-4.29 (m, 1H), 3.81 (s, 3H), 3.61-3.40 (m, 4H), 3.31-3.26 (m, 3H), 2.77 (br d, J = 6.5 Hz, 2H), 2.22-2.06 (m, 2H), 1.85 (d, J = 13.2 Hz, 2H), 1.65- 1.53 (m, 2H), 1.42 (t, J = 12.9 Hz, 2H), 0.78-0.61 (m, 1H), 0.27 (d, J = 7.8 Hz, 2H),-0.13 (d, J = 4.6 Hz, 2H) | 515.55 |
| 32 | 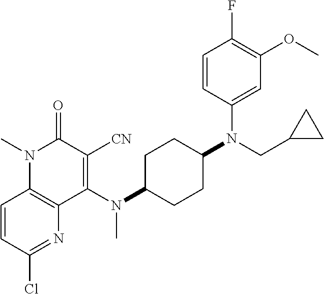 | (300 MHz, DMSO-d6) δ 8.06 (d, J = 8.9 Hz, 1H), 7.80 (d, J = 8.9 Hz, 1H), 7.08 (t, J = 9.3 Hz, 1H), 6.90 (d, J = 7.6 Hz, 1H), 6.69 (d, J = 8.3 Hz, 1H), 6.55 (s, 1H), 4.46-4.30 (m, 1H), 3.82 (s, 3H), 3.53 (s, 3H), 3.48-3.41 (m, 1H), 3.26 (s, 3H), 2.91 (d, J = 6.0 Hz, 1H), 2.29-2.03 (m, 2H), 1.92-1.79 (m, 2H), 1.76-1.61 (m, 2H), 1.55-1.38 (m, 2H), 0.80- 0.65 (m, 1H), 0.33 (d, J = 7.8 Hz, 2H), −0.03 (d, J = 4.6 Hz, 2H) | 524.5 |
| 33 | 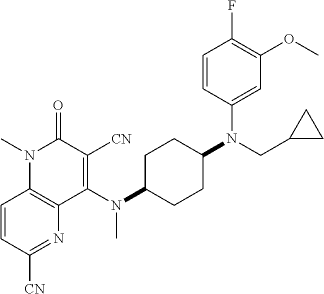 | (300 MHz, DMSO-d6) δ = 8.25 (d, J = 8.7 Hz, 2H), 8.18-8.09 (m, 2H), 7.13-7.02 (m, 2H), 6.92 (d, J = 7.7 Hz, 2H), 6.75-6.65 (m, 2H), 4.45-4.33 (m, 1H), 3.82 (s, 3H), 3.54 (s, 3H), 3.29 (s, 3H), 2.90 (d, J = 6.2 Hz, 5H), 2.14 (q, J = 10.9 Hz, 2H), 1.94- 1.79 (m, 2H), 1.68 (d, J = 10.1 Hz, 4H), 1.57-1.38 (m, 2H), 0.79-0.66 (m, 2H), 0.33 (d, J = 8.0 Hz, 4H), −0.04 (d, J = 4.8 Hz, 4H) | 515.5 |
| 34 | 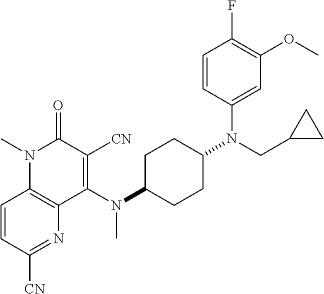 | (300 MHz, DMSO-d6) δ = 8.26 (d, J = 9.0 Hz, 1H), 8.15 (d, J = 9.0 Hz, 1H), 7.00 (t, J = 10.2 Hz, 1H), 6.57 (d, J = 6.9 Hz, 1H), 6.37 (d, J = 9.1 Hz, 1H), 4.45-4.31 (m, 1H), 3.84 (s, 3H), 3.54 (s, 4H), 3.02 (d, J = 5.6 Hz, 2H), 2.05 (s, 4H), 1.85 (d, J = 12.1 Hz, 2H), 1.70-1.48 (m, 2H), 0.98-0.82 (m, 1H), 0.47 (d, J = 8.0 Hz, 2H), 0.23 (d, J = 4.7 Hz, 2H) | 515.5 |
| 35 | 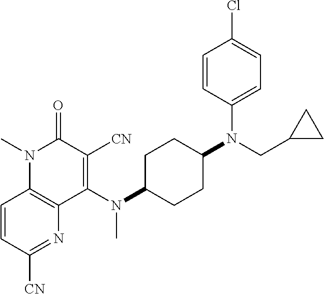 | (300 MHz, DMSO-d6) δ = 8.27 (d, J = 8.7 Hz, 1H), 8.18 (d, J = 8.7 Hz, 1H), 7.26 (d, J = 8.4 Hz, 2H), 7.06 (d, J = 8.5 Hz, 2H), 4.47-4.35 (m, 1H), 3.56 (s, 4H), 3.26 (s, 3H), 3.00 (d, J = 6.1 Hz, 2H), 2.17-2.02 (m, 2H), 1.87-1.63 (m, 4H), 1.50 (t, J = 11.0 Hz, 2H), 0.98-0.88 (m, 1H), 0.35 (d, J = 8.0 Hz, 2H), 0.01 (d, J = 4.9 Hz, 2H) | 501.48 |
| 36 | 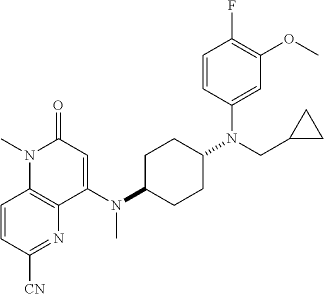 | (300 MHz, DMSO-d6) δ = 8.15 (d, J = 8.4 Hz, 1H), 8.04 (d, J = 8.4 Hz, 1H), 6.99 (t, J = 10.1 Hz, 1H), 6.56 (d, J = 6.2 Hz, 1H), 6.35 (d, J = 8.8 Hz, 1H), 5.88 (s, 1H), 4.42-4.26 (m, 1H), 3.83 (s, 3H), 3.64-3.43 (m, 4H), 3.02 (d, J = 5.4 Hz, 2H), 2.88 (s, 3H), 1.98-1.74 (m, 6H), 1.65-1.45 (m, 2H), 0.96-0.83 (m, 1H), 0.47 (d, J = 8.0 Hz, 2H), 0.22 (d, J = 4.8 Hz, 2H) | 490.5 |
| 37 | 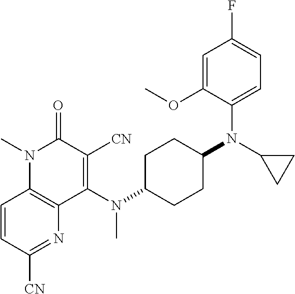 | (300 MHz, DMSO-d6) δ 8.25 (d, J = 8.8 Hz, 1H), 8.13 (d, J = 8.9 Hz, 1H), 7.05 (dd, J = 8.7, 6.5 Hz, 1H), 6.82 (dd, J = 11.1, 2.9 Hz, 1H), 6.65 (td, J = 8.4, 2.8 Hz, 1H), 4.36-4.25 (m, 1H), 3.77 (s, 3H), 3.53 (s, 3H), 3.23 (s, 3H), 3.18-3.10 (m, 1H), 2.57- 2.50 (m, 1H), 2.04-1.70 (m, 6H), 1.70-1.51 (m, 2H), 0.58-0.52 (m, 2H), 0.23-0.17 (m, 2H) | 501.20 |
| 38 | 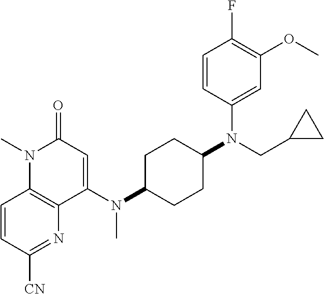 | (300 MHz, DMSO-d6) δ = 8.15 (d, J = 8.9 Hz, 1H), 8.04 (d, J = 8.9 Hz, 1H), 7.14-7.06 (m, 1H), 6.95 (d, J = 7.6 Hz, 1H), 6.79-6.59 (m, 1H), 5.94 (s, 1H), 4.39-4.23 (m, 1H), 3.82 (s, 3H), 3.52 (s, 3H), 2.95-2.85 (m, 4H), 2.10-1.86 (m, 4H), 1.59- 1.38 (m, 4H), 0.76-0.65 (m, 1H), 0.33 (d, J = 7.8 Hz, 2H), −0.05 (d, J = 4.7 Hz, 2H) | 490.7 |
| 39 | 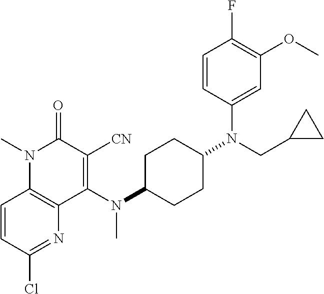 | (300 MHz, DMSO-d6) δ = 8.06 (d, J = 9.0 Hz, 1H), 7.81 (d, J = 8.9 Hz, 1H), 7.00 (t, J = 10.2 Hz, 1H), 6.57 (d, J = 7.1 Hz, 1H), 6.37 (d, J = 8.7 Hz, 1H), 4.41-4.26 (m, 1H), 3.83 (s, 3H), 3.53 (s, 3H), 3.26 (s, 3H), 3.02 (d, J = 5.4 Hz, 2H), 2.12-1.93 (m, 4H), 1.85 (d, J = 11.7 Hz, 2H), 1.69-1.49 (m, 2H), 0.95-0.83 (m, 1H), 0.47 (d, J = 8.0 Hz, 1H), 0.22 (d, J = 4.8 Hz, 1H) | 524.5 |
| 40 | 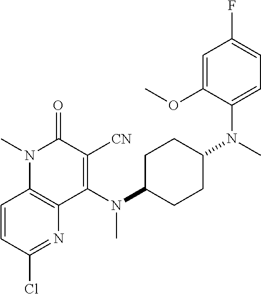 | 1HNMR (400 MHz, DMSO) δ 8.05 (d, J = 9.0 Hz, 1H), 7.79 (d, J = 8.9 Hz, 1H), 6.92 (dd, J = 8.6, 6.5 Hz, 1H), 6.83 (dd, J = 11.0, 2.8 Hz, 1H), 6.65 (td, J = 8.4, 2.7 Hz, 1H), 4.31-4.17 (m, 1H), 3.80 (s, 3H), 3.52 (s, 3H), 3.20 (s, J = 14.0 Hz, 3H), 3.08 (t, J = 11.8 Hz, 1H), 2.57 (s, J = 14.1 Hz, 3H), 2.08- 1.97 (m, 2H), 1.87-1.73 (m, 4H), 1.60-1.44 (m, 2H) | 484.48 |
| 41 | 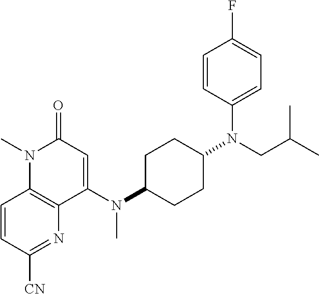 | (300 MHz, CDCl₃) δ 7.75 (d, J = 8.7 Hz, 1H), 7.69 (d, J = 8.7 Hz, 1H), 7.01-6.90 (m, 2H), 6.89-6.79 (m, 2H), 6.04 (s, 1H), 4.31 (bt, J = 11.3 Hz, 1H), 3.64 (s, 3H), 3.34 (bt, J = 11.3 Hz, 1H), 2.95 (s, 3H), 2.89-2.77 (m, 2H), 2.15-1.90 (m, 4H), 1.85- 1.68 (m, 3H), 1.68-1.43 (m, 2H), 0.89 (br d, J = 6.5 Hz, 6H) | — |
| 42 | 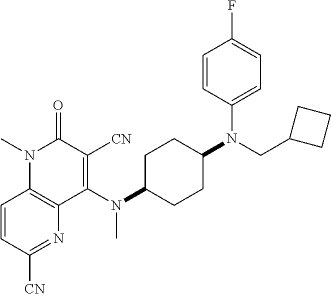 | (300 MHz, CDCl₃) δ 7.85 (br d, J = 8.7 Hz, 1H), 7.72 (br d, J = 8.9 Hz, H), 7.18-7.09 (m, 2H), 7.09- 6.93 (m, 2H), 4.60-4.42 (m, 1H), 3.65 (s, 3H), 3.46 (s, 3H), 3.20 (br s, 1H), 2.94 (d, J = 7.1 Hz, 2H), 2.39-2.13 (m, 3H), 2.09-1.96 (m, 2H), 1.95-1.82 (m, 3H), 1.81-1.67 (m, 3H), 1.65-1.42 (m, 3H), 1.33-1.18 (m, 1H) | — |
| 43 | 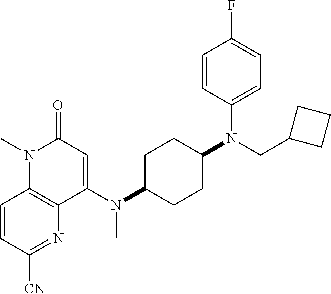 | (300 MHz, CDCl₃) δ 7.78 (d, J = 8.8 Hz, 1H), 7.67 (d, J = 8.7 Hz, 1H), 7.18-7.08 (m, 2H), 7.06-6.96 (m, 2H), 6.04 (s, 1H), 4.52-4.37 (m, 1H), 3.63 (s, 3H), 3.18 (br s, 1H), 3.02 (s, 3H), 2.95 (d, J = 7.2 Hz, 2H), 2.29-2.03 (m, 3H), 2.02-1.70 (m, 7H), 1.68-1.36 (m, 5H) | — |
| 44 | 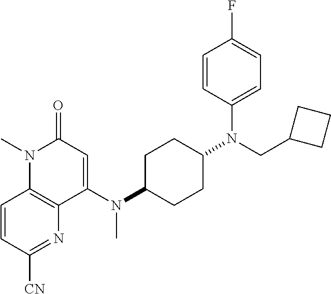 | (300 MHz, CDCl₃) δ 7.81 (d, J = 8.8 Hz, 1H), 7.70 (d, J = 8.7 Hz, 1H), 7.00-6.88 (m, 2H), 6.83-6.72 (m, 2H), 6.05 (s, 1H), 4.44-4.28 (m, 1H), 3.65 (s, 3H), 3.32 (bt, J = 11.6 Hz, 1H), 3.10 (d, J = 6.4 Hz, 2H), 2.96 (s, 3H), 2.51-2.36 (m, 1 H), 2.15-2.03 (m, 2H), 2.02-1.90 (m, 4H), 1.88-1.74 (m, 4H), 1.73-1.61 (m, 2H), 1.60-1.41 (m, 2H), 1.39-1.08 (m, 1H) | — |
| 45 | 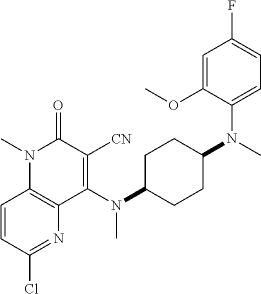 | (400 MHz, DMSO) δ 7.97 (d, J = 9.0 Hz, 1H), 7.71 (d, J = 8.9 Hz, 1H), 7.17-7.07 (m, 1H), 6.82 (dt, J = 17.1, 8.5 Hz, 1H), 6.68-6.60 (m, 1H), 4.27 (t, J = 11.4 Hz, 1H), 3.75 (s, 3H), 3.44 (s, 3H), 3.20 (s, 3H), 3.17 (s, 1H), 2.50 (s, 3H), 2.06 (dd, J = 22.7, 10.7 Hz, 2H), 1.76 (d, J = 13.6 Hz, 2H), 1.55 (d, J = 9.6 Hz, 2H), 1.33 (t, J = 13.3 Hz, 2H) | 484.2 |
| 46 | 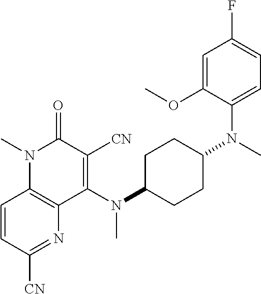 | 1HNMR (400 MHz, DMSO) δ 8.25 (d, J = 8.7 Hz, 1H), 8.13 (d, J = 8.9 Hz, 1H), 6.99-6.88 (m, 1H), 6.86-6.78 (m, 1H), 6.68-6.61 (m, 1H), 4.36-4.19 (m, 1H), 3.80 (s, 3H), 3.53 (s, 3H). 3.23 (s, 3H), 3.13-3.03 (m, 1H), 2.57 (s, 3H), 2.08-1.94 (m, 2H), 1.94-1.71 (m, 4H), 1.58-1.44 (m, 2H) | 475.51 |
| 47 | 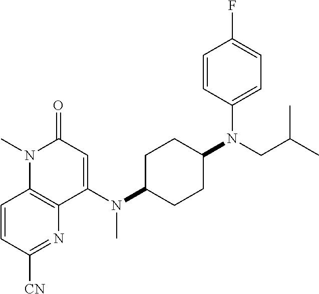 | (300 MHz, CDCl₃) δ 7.78 (d, J = 8.7 Hz, 1H), 7.68 (d, J = 8.7 Hz, 1H), 7.25-7.15 (m, 2H), 7.09-6.97 (m, 2H), 6.05 (s, 1H), 4.52-4.38 (m, 1H), 3.64 (s, 3H), 3.10 (br s, 1H), 3.03 (s, 3H), 2.69 (d, J = 6.9 Hz, 2 H), 2.15 (q, J = 11.8 Hz, 2H), 1.94 (bd, J = 13.5 Hz, 2H), 1.60-1.30 (m, 4H), 1.14 (br s, 1H), 0.92 (d, J = 6.5 Hz, 6H) | 462.09 |
| 48 | 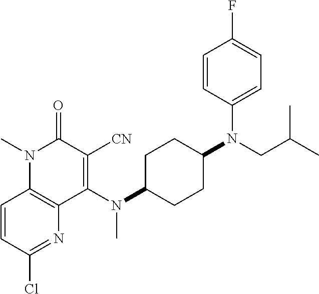 | (300 MHz, CDCl₃) δ 7.63 (d, J = 8.7 Hz, 1H), 7.50 (d, J = 8.7 Hz, 1H), 7.25-7.12 (m, 2H), 7.10-6.94 (m, 2H), 4.58-4.44 (m, 1H), 3.63 (s, 3H), 3.43 (s, 3H), 3.17-3.05 (m, 1H), 2.75-2.54 (m, 2H), 2.43- 2.03 (m, 2H), 2.02-1.67 (m, 4H) 1.58-1.31 (m, 2H), 1.14 (br s, 1H) 0.91 (br d, J = 5.9 Hz, 6H) | 497.27 |
| 49 | 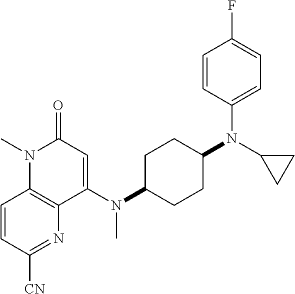 | (300 MHz, DMSO-d6) δ = 8.13 (d, J = 9.0 Hz, 1H), 8.03 (d, J = 9.0 Hz, 1H), 7.24-7.06 (m, 4H), 5.91 (s, 1H), 4.35-4.21 (m, 1H), 3.51 (s, 3H), 3.29 (br s, 1H), 2.88 (s, 3H), 2.34-2.18 (m, 1H), 2.10- 1.88 (m, 4H), 1.61-1.35 (m, 4H), 0.55 (d, J = 5.3 Hz, 2H), 0.48-0.32 (m, 2H) | 446.4 |
| 50 | 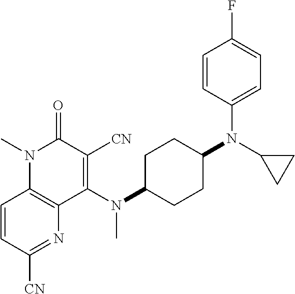 | (300 MHz, DMSO-d6) δ = 8.25 (d, J = 8.4 Hz, 1H), 8.15 (d, J = 9.0 Hz, 1H), 7.24-7.05 (m, 4H), 4.43-4.31 (m, 1H), 3.54 (s, 3H), 3.28 (s, 3H), 2.33- 2.24 (m, 1H), 2.13 (q, J = 10.2 Hz, 2H), 2.04-1.90 (m, 2H), 1.67 (d, J = 10.3 Hz, 2H), 1.58-1.42 (m, 2H), 0.58 (d, J = 5.8 Hz, 1H), 0.44-0.35 (m, 1H) | 471.3 |
| 51 | 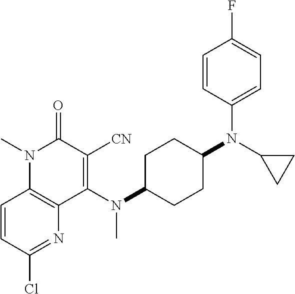 | (300 MHz, DMSO-d6) δ = 8.06 (d, J = 9.0 Hz, 1H), 7.79 (d, J = 8.8 Hz, 1H), 7.25-7.02 (m, 4H), 4.41-4.27 (m, 1H), 3.53 (s, 3H), 3.25 (s, 3H), 2.28 (br s, 1H), 2.19-2.03 (m, 2H), 1.97 (d, J = 11.6 Hz, 2H), 1.75-1.61 (m, 2H), 1.56-1.41 (m, 2H), 0.57 (d, J = 5.4 Hz, 1H), 0.39 (br s, 1H) | 481.3 |
| 52 | 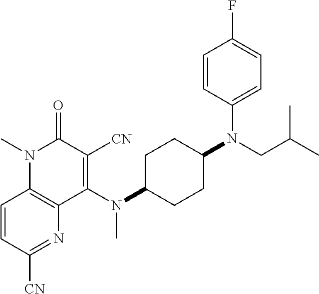 | (300 MHz, CDCl₃) δ 7.84 (d, J = 8.8 Hz, 1H), 7.71 (d, J = 8.7 Hz, 1H), 7.23-7.13 (m, 2H), 7.13-6.91 (m, 2 H), 4.58-4.43 (m, 1H), 3.64 (s, 3H), 3.45 (s, 3H), 3.10 (br s, 1H), 2.67 (d, J = 6.9 Hz, 2H), 2.39- 2.18 (m, 2H), 1.96 (bd, J = 13.8 Hz, 2H), 1.75 (bd, J = 10.4 Hz, 2H), 1.60-1.30 (m, 3H), 0.91 (d, J = 6.4 Hz, 6H) | 487.13 |
| 53 | 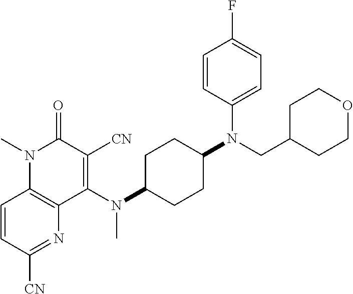 | (300 MHz, CDCl₃) δ 7.86 (d, J = 8.5 Hz, 1H), 7.73 (d, J = 8.7 Hz, 1H), 7.18 (br s, 2H), 7.11-6.95 (m, 2H), 4.52 (bt, J = 10.6 Hz, 1H), 3.95 (br d, J = 10.5 Hz, 2H), 3.65 (s, 3H), 3.45 (br s, 3H), 3.29 (bt, J = 11.2 Hz, 2H), 3.13 (br s, 1H), 2.85-2.70 (m, 2H), 2.34-2.13 (m, 2H), 1.87-1.63 (m, 4H), 1.62-1.43 (m, 2H), 1.42-1.13 (m, 3H) | 529.45 |
| 54 | 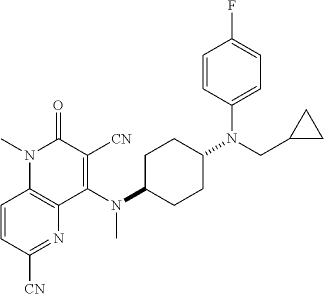 | 1H NMR (300 MHz, DMSO-d6) δ = 8.27 (d, J = 9.0 Hz, 1H), 8.14 (d, J = 8.9 Hz, 1H), 7.03 (t, J = 8.5 Hz, 2H), 6.92-6.82 (m, 2H), 4.46-4.25 (m, 1H), 3.69-3.59 (m, 1H), 3.54 (s, 3H), 3.31-3.25 (m, 3H), 3.01 (d, J = 5.3 Hz, 2H), 2.12-1.98 (m, 4H), 1.84 (d, J = 11.8 Hz, 2H), 1.70-1.46 (m, 2H), 0.95-0.82 (m, 1H), 0.45 (d, J = 7.9 Hz, 2H), 0.21 (d, J = 4.5 Hz, 2H) | 486.3 |
| 55 | 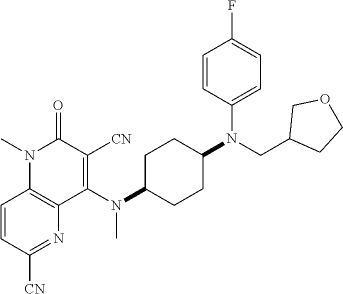 | 1H NMR (300 MHz, DMSO-d6) δ = 8.24 (d, J = 8.8 Hz, 1H), 8.13 (d, J = 8.9 Hz, 1H), 7.33-7.22 (m, 2H), 7.22-7.09 (m, 2H), 4.44-4.31 (m, 1H), 3.74- 3.61 (m, 2H), 3.53 (s, 3H), 3.32-3.25 (m, 4H), 3.17 (br s, 2H), 2.92 (d, J = 6.9 Hz, 2H), 2.24-2.05 (m, 2H), 1.98-1.60 (m, 6H), 1.60-1.33 (m, 3H) | 515.5 |
| 56 | 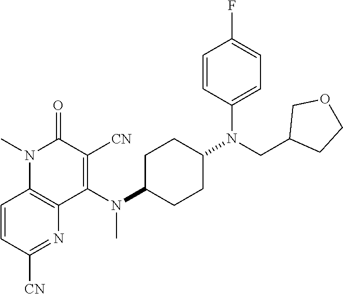 | 1H NMR (300 MHz, DMSO-d6) δ = 8.26 (d, J = 8.7 Hz, 1H), 8.14 (d, J = 8.7 Hz, 1H), 7.08-6.87 (m, 4H), 4.40-4.22 (m, 1H), 3.82-3.68 (m, 1H), 3.62- 3.41 (m, 4H), 3.27-3.17 (m, 3H), 3.06-2.93 (m, 2H), 2.54 (s, 2H), 2.44-2.21 (m, 2H), 2.10-1.74 (m, 8H), 1.71-1.43 (m, 4H) | 516.2 |
| 57 | 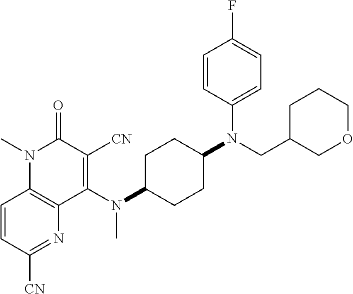 | 1H NMR (300 MHz, DMSO-d6) δ = 8.25 (d, J = 8.8 Hz, 1H), 8.14 (d, J = 8.9 Hz, 1H), 7.32-7.21 (m, 1H), 7.25-7.19 (m, 1H), 7.19-7.08 (m, 2H), 4.44- 4.29 (m, 1H), 3.88 (d, J = 9.4 Hz, 1H), 3.67 (d, J = 10.7 Hz, 1H), 3.54 (s, 3H), 3.32-3.22 (m, 5H), 3.20-3.00 (m, 2H), 2.91-2.79 (m, 1H), 2.77-2.68 (m, 1H), 2.29-2.00 (m, 2H), 1.96-1.77 (m, 1H), 1.76-1.59 (m, 4H), 1.56-1.29 (m, 5H), 1.29-1.04 (m, 1H) | 530.6 |
| 58 | 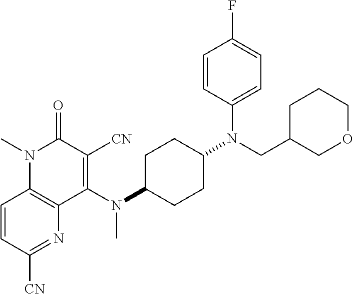 | 1H NMR (300 MHz, DMSO-d6) δ = 8.26 (d, J = 8.9 Hz, 1H), 8.14 (d, J = 9.0 Hz, 1H), 7.03 (t, J = 8.5 Hz, 2H), 6.95-6.84 (m, 2H), 4.33 (s, 1H), 3.69 (t, J = 12.7 Hz, 3H), 3.54 (s, 3H), 3.26 (s, 3H),2.91 (d, J = 6.6 Hz, 1H), 2.11-1.87 (m, 3H), 1.86-1.69 (m, 2H), 1.69-1.45 (m, 3H), 1.45-1.28 (m, 1H), 1.27-1.10 (m, 1H) | 530.7 |
| 59 | 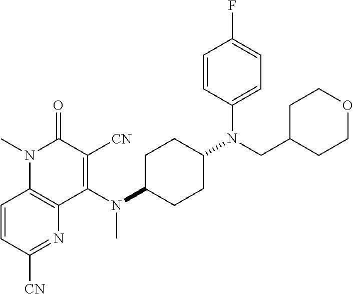 | (300 MHz, CDCl₃) δ 7.88 (d, J = 8.8 Hz, 1H), 7.74 (d, J = 8.9 Hz, 1H), 7.08-6.90 (m, 2H), 6.90-6.81 (m, 2H), 4.37 (bt, J = 9.8 Hz, 1H), 3.94 (bd, J = 9.4 Hz, 2H), 3.66 (s, 3H), 3.38 (s, 3H), 3.36-3.18 (m, 3H), 2.99-2.85 (m, 2H), 2.26-2.12 (m, 2H), 2.10- 1.77 (m, 5H), 1.74-1.62 (m, 3H), 1.36-1.14 (m,3H) | 529.34 |
| 60 | 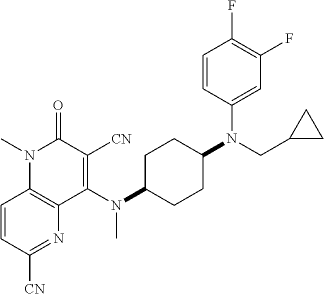 | (400 MHz, DMSO-d6) δ ppm: 8.26 (d, J = 8.9 Hz, 1H), 8.16 (d, J = 9.0 Hz, 1H), 7.27 (dd, J = 19.9, 9.4 Hz, 1H), 7.19-7.07 (m, 1H), 6.93-6.84 (m, 1H), 4.44-4.35 (m, 1H), 3.55 (s, 3H), 3.53-3.47 (m, 1H), 3.26 (s, 3H), 2.96 (d, J = 6.3 Hz, 2H), 2.09 (dd, J = 19.2, 9.5 Hz, 2H), 1.85-1.75 (m, 2H), 1.75-1.64 (m, 2H), 1.56-1.42 (m, 2H), 0.80-0.68 (m, 1H), 0.39- 0.29 (m, 2H), 0.02-−0.04 (m, 2H) | 503.37 |
| 61 | 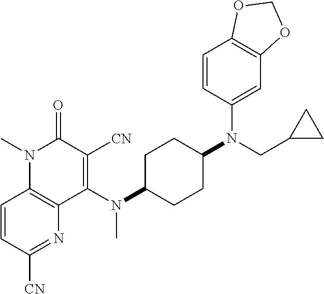 | (400 MHz, DMSO) δ 8.24 (d, J = 8.9 Hz, 1H), 8.13 (d, J = 9.0 Hz, 1H), 6.85 (dd, J = 12.8,5.1 Hz, 2H), 6.70 (dd, J = 8.2, 2.0 Hz, 1H), 5.98 (s, 2H), 4.43- 4.32 (m, 1H), 3.53 (s, 3H), 3.30-3.23 (m, 4H), 2.79 (d, J = 6.6 Hz, 2H), 2.21-2.07 (m, 2H), 1.89 (d, J = 12.0 Hz, 2H), 1.64 (d, J = 8.0 Hz, 2H), 1.51-1.37 (m, 2H), 0.73-0.60 (m, 1H), 0.36-0.25 (m, 2H), 0.01-−0.09 (m, 2H) | 511.39 |
| 62 | 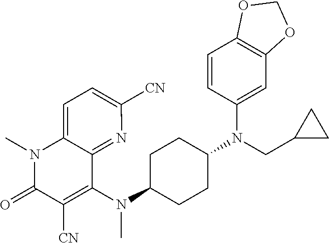 [SMILES: Cn1c(=O)c(C#N)c(N(C)[C@H]2CC[C@H](N(CC3CC3)c3ccc4c(c3)OCO4)CC2)c2nc(C#N)ccc21] | 1HNMR (400 MHz, DMSO-d6) δ 8.26 (d, J = 8.9 Hz, 1H), 8.13 (d, J = 9.0 Hz, 1H), 5.99 (s, 1H), 5.99 (s, 1H), 5.87-5.83 (m, 1H), 4.44-4.33 (m, 1H), 3.71 (s, 6H), 3.69-3.62 (m, 1H), 3.54 (s, 3H), 3.32 (s, J = 4.8 Hz, 3H), 3.01 (d, J = 5.6 Hz, 2H), 2.14- 2.01 (m, 4H), 1.88-1.77 (m, 2H), 1.69-1.53 (m, 2H), 0.99-0.84 (m, 1H), 0.54-0.42 (m, 2H), 0.28- 0.19 (m, 2H) | 527.30 |
| 63 | 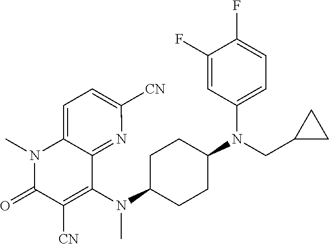 [SMILES: Cn1c(=O)c(C#N)c(N(C)[C@H]2CC[C@@H](N(CC3CC3)c3ccc(F)c(F)c3)CC2)c2nc(C#N)ccc21] | 1HNMR (400 MHz, DMSO-d6) δ 8.07 (d, J = 9.0 Hz, 1H), 7.80 (d, J = 8.9 Hz, 1H), 7.80 (d, J = 8.9 Hz, 1H), 7.26 (dd, J = 19.7, 9.5 Hz, 1H), 7.08 (ddd, J = 13.7, 7.2, 2.5 Hz, 1H), 6.85 (d, J = 9.0 Hz, 1H), 4.37 (s, 1H), 3.53 (d, J = 9.4 Hz, 4H), 3.23 (s, 3H), 2.96 (d, J = 6.2 Hz, 2H), 2.07 (dd, J = 18.7, 9.0 Hz, 2H), 1.74 (dd, J = 22.2, 8.5 Hz, 4H), 1.54-1.40 (m, 2H), 0.80-0.68 (m, 1H), 0.34 (q, J = 5.4 Hz, 2H), (−)0.01 (q, J = 5.1 Hz, 2H) | 512.22 |
| 64 | 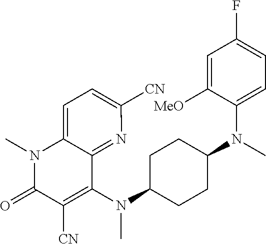 [SMILES: COc1cc(F)ccc1N(C)[C@H]1CC[C@@H](N(C)c2c(C#N)c(=O)n(C)c3ccc(C#N)nc23)CC1] | 1HNMR (400 MHz, DMSO-d6) δ 8.24 (d, J = 8.9 Hz, 1H), 8.12 (d, J = 8.9 Hz, 1H), 7.22 (dd, J = 8.6, 6.7 Hz, 1H), 6.90 (dd, J = 11.2, 2.8 Hz, 1H), 6.70 (td, J = 8.4, 2.8 Hz, 1H), 4.42-4.30 (m, 1H), 3.82 (s, 3H), 3.52 (s, 3H), 3.30 (s, 3H), 3.27-3.21 (m, 1H), 2.57 (s, 3H), 2.21-2.07 (m, 2H), 1.88-1.78 (m, 2H), 1.61 (dd, J = 11.2, 2.5 Hz, 2H), 1.46-1.36 (m, 2H) | 475.24 |
| 65 | 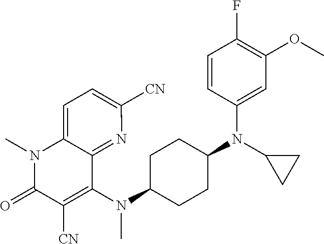 | 1HNMR (400 MHz, DMSO-d6) δ 8.25 (d, J = 8.9 Hz, 1H), 8.14 (d, J = 8.9 Hz, 1H), 7.07 (dd, J = 11.4, 8.7 Hz, 1H), 6.92 (dd, J = 8.0, 2.2 Hz, 1H), 6.72-6.63 (m, 1H), 4.37 (t, J = 9.4 Hz, 1H), 3.81 (s, 3H), 3.54 (s, 3H), 3.35 (s, 1H), 3.28 (s, 2H), 2.33- 2.26 (m, 1H), 2.13 (dd, J = 23.0, 11.6 Hz, 2H), 1.99 (d, J = 12.2 Hz, 2H), 1.68 (d, J = 8.9 Hz, 2H), 1.50 (td, J = 10.8, 2.9 Hz, 2H), 0.62-0.55 (m, 2H), 0.43- 0.39 (m, 2H) | 501.26 |
| 66 | 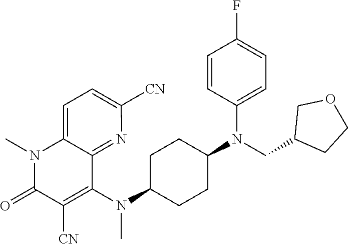 | 1HNMR (400 MHz, CDCl3) δ 7.84-7.81 (d, J = 8.8 Hz, 1H), 7.71-7.69 (d, J = 8.8 Hz, 1H), 7.16 (m, 2H), 7.06-6.99 (m, 2H), 4.52-4.47 (t, J = 11.2, 10.8 Hz, 1H), 3.83-3.69 (m, 2H), 3.67-3.52 (m, 4H), 3.50-3.43 (m, 4H), 3.11-2.92 (s, 1H), 2.87 (s, 2H), 2.23-2.22 (m, 2H), 2.03-1.92 (m, 4H), 1.89-1.79 (m, 2H), 1.62-1.33 (m, 3H), 1.28-1.19 (s,1H), 0.89-0.83 (m,1H) | 515.25 |
| 67 | 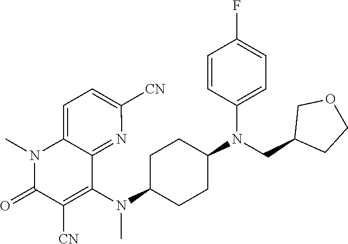 [SMILES: Cn1c(=O)c(C#N)c(N(C)[C@H]2CC[C@@H](N(C[C@@H]3CCOC3)c3ccc(F)cc3)CC2)c2nc(C#N)ccc21] | 1HNMR (400 MHz, CDCl3) δ 7.84-7.81 (d, J = 8.8 Hz, 1H), 7.71-7.69 (d, J = 8.8 Hz, 1H), 7.16 (m, 2H), 7.06-7.02 (m, 2H), 4.52-4.49 (m, 1H), 3.83- 3.65 (m, 6H), 3.56-3.21 (m, 4H), 3.21-3.11 (s, 1H), 2.92-2.91 (m, 2H), 2.23-2.20 (m, 2H), 2.03-2.00 (m, 2H), 1.92-1.89 (m, 2H), 1.81-1.60 (m, 3H), 1.56-1.39 (m, 2H), 1.33-1.25 (m, 2H), 0.98-0.83 (m, 1H) | 515.25 |
| 68 | 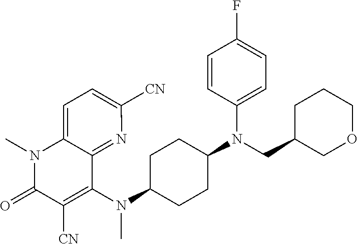 [SMILES: Cn1c(=O)c(C#N)c(N(C)[C@H]2CC[C@@H](N(C[C@H]3CCCOC3)c3ccc(F)cc3)CC2)c2nc(C#N)ccc21] | 1HNMR (400 MHz, CDCl3) δ 7.84-7.81 (d, J = 8.8 Hz, 1H), 7.71-7.69 (d, J = 8.8 Hz, 1H), 7.16 (m, 2H), 7.04-7.00 (m, 2H), 4.48 (m, 1H), 4.01-3.99 (d, J = 9.2 Hz, 1H), 3.90-3.79 (m, 1H), 3.63-3.54 (s, 3H), 3.43-3.25 (m, 4H), 3.18-3.08 (m, 2H), 2.92- 2.72 (m, 2H), 2.34-2.00 (m, 3H), 1.87-1.72 (m, 4H), 1.69-1.38 (m, 6H), 1.35-1.19 (m, 2H), 0.99- 0.85 (m, 1H) | 529.25 |
| 69 | 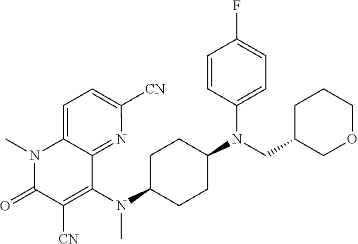 [SMILES: Cn1c(=O)c(C#N)c(N(C)[C@H]2CC[C@@H](N(C[C@@H]3CCCOC3)c3ccc(F)cc3)CC2)c2nc(C#N)ccc21] | 1HNMR (400 MHz, CDCl₃) δ 8.08-8.05 (d, J = 10.4 Hz, 1H), 8.03-8.02 (d, J = 2.4 Hz, 1H), 7.16 (s, 2H), 7.02-6.88 (m, 2H), 4.59-4.29 (m, 1H), 4.01-3.99 (m, 1H), 3.89-3.62 (m, 1H), 3.53 (s, 3H), 3.42-3.39 (m, 4H), 3.18-3.08 (m, 2H), 2.95-2.63 (m, 2H), 2.45-1.72 (m, 8H), 1.56-1.22 (m, 9H), 0.98-0.87 (m, 1H) | 529.25 |
| 70 | 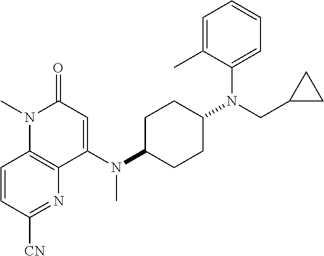 | (300 MHz, DMSO-d6) δ = 8.11 (d, J = 8.7 Hz. 1H), 8.01 (d, J = 8.7 Hz, 1H), 7.26-7.08 (m, 3H), 6.96 (t, J = 7.3 Hz, 1H), 5.83 (s, 1H), 4.23 (t, J = 10.9 Hz, 1H), 3.95-3.63 (m, 2H), 2.90-2.65 (m, 6H), 2.26 (s, 3H), 2.07 (s, 1H), 1.90-1.77 (m, 4H), 1.77-1.56 (m, 2H), 1.37 (q, J = 11.3 Hz, 2H), 0.65- 0.53 (m, 1H), 0.23 (d, J = 7.7 Hz, 2H), −0.05 (d, J = 4.6 Hz, 2H) | 456.6 |
| 71 | 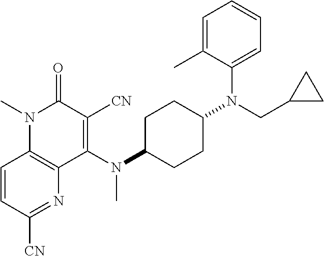 | (300 MHz, DMSO-d6) δ = 8.25 (d, J = 9.0 Hz, 1H), 8.13 (d, J = 8.9 Hz, 1H), 7.27-7.09 (m, 3H), 6.98 (t, J = 7.0 Hz, 1H), 4.36-4.21 (m, 1H), 3.53 (s, 3H), 3.22 (s, 3H), 2.91-2.69 (m, 3H), 2.29 (s, 3H), 2.03-1.73 (m, 6H), 1.54-1.36 (m, 2H), 0.71-0.52 (m, 1H), 0.25 (d, J = 7.8 Hz, 2H),-0.03 (d, J = 4.5 Hz, 2H) | 481.6 |
| 72 | 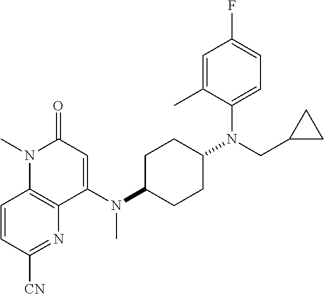 | (300 MHz, DMSO-d6) δ = 8.13 (d, J = 8.8 Hz, 1H), 8.02 (d, J = 8.9 Hz, 1H), 7.28-7.19 (m, 1H), 7.02 (d, J = 9.6 Hz, 1H), 6.94 (t, J = 8.3 Hz, 1H), 5.84 (s, 1H), 4.31-4.15 (m, 1H), 3.50 (s, 3H), 2.89- 2.76 (m, 1H), 2.74-2.62 (m, 1H), 2.28 (s, 3H), 1.92-1.77 (m, 4H), 1.66 (q, J = 11.5 Hz, 2H), 1.48- 1.19 (m, 2H), 0.64-0.51 (m, 1H), 0.24 (d, J = 7.8 Hz, 2H), −0.06 (d, J = 4.5 Hz, 2H) | 474.6 |
| 73 | 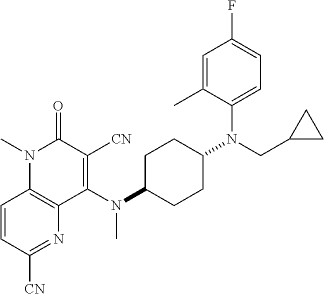 | (300 MHz, DMSO-d6) δ = 8.25 (d, J = 8.9 Hz, 1H), 8.12 (d, J = 9.0 Hz, 1H), 7.30-7.20 (m, 1H), 7.03 (d, J = 9.4 Hz, 1H), 6.95 (t, J = 8.3 Hz, 1H), 4.27 (t, J = 9.8 Hz, 1H), 3.52 (s, 3H), 3.29-3.14 (m, 3H), 2.92-2.68 (m, 3H), 2.29 (s, 3H), 2.08-1.75 (m, 6H), 1.51-1.23 (m, 2H), 0.66-0.52 (m, 1H), 0.25 (d, J = 7.8 Hz, 2H), −0.05 (d, J = 4.5 Hz, 2H) | 499.6 |
| 74 | 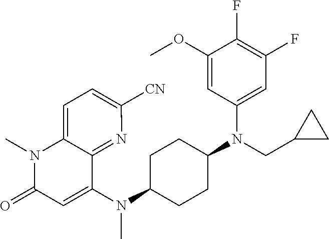 [SMILES: COc1cc(N(CC2CC2)[C@H]2CC[C@@H](N(C)c3cc(=O)n(C)c4ccc(C#N)nc34)CC2)cc(F)c1F] | 1HNMR (400 MHz, DMSO-d6) δ 8.27 (d, J = 8.9 Hz, 1H), 8.16 (d, J = 9.0 Hz, 1H), 6.68-6.56 (m, 2H), 4.45-4.34 (m, 1H), 3.85 (s, 3H), 3.55 (s, 3H), 3.55-3.51 (m, 1H), 3.27 (s, 3H), 2.98 (d, J = 6.2 Hz, 2H), 2.19-2.04 (m, 2H), 1.86-1.66 (m, 4H), 1.58-1.43 (m, 2H), 0.82-0.71 (m, 1H), 0.40-0.33 (m, 2H), 0.05-(−)0.00 (m, 2H) | 533.26 |
| 75 | 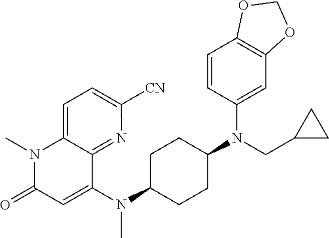 [SMILES: Cn1c(=O)cc(N(C)[C@H]2CC[C@@H](N(CC3CC3)c3ccc4c(c3)OCO4)CC2)c2nc(C#N)ccc21] | 1HNMR (400 MHz, DMSO-d6) δ 8.13 (d, J = 8.8 Hz, 1H), 8.03 (d, J = 8.9 Hz, 1H), 6.86 (d, J = 1.9 Hz, 1H), 6.83 (d, J = 8.2 Hz, 1H), 6.70 (dd, J = 8.2, 2.0 Hz, 1H), 5.98 (s, 2H), 5.89 (s, 1H), 4.34 (tt, J = 11.4, 3.8 Hz, 1H), 3.51 (s, 3H), 3.28-3.21 (m, 1H), 2.89 (s, 3H), 2.77 (d, J = 6.6 Hz, 2H), 2.08-1.95 (m, 2H), 1.94-1.83 (m, 2H), 1.50 (dd, J = 12.2, 2.9 Hz, 2H), 1.44-1.33 (m, 2H), 0.71-0.60 (m, 1H), 0.34-0.26 (m, 2H), (−)0.06 (q, J = 4.9 Hz, 2H) | 486.27 |
| 76 | 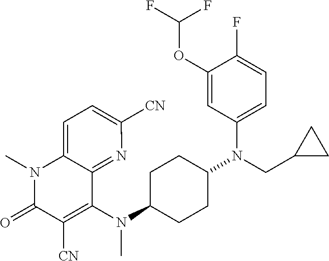 [SMILES: Cn1c(=O)c(C#N)c(N(C)[C@H]2CC[C@H](N(CC3CC3)c3ccc(F)c(OC(F)F)c3)CC2)c2nc(C#N)ccc21] | 1HNMR (400 MHz, DMSO-d6) δ 8.26 (d, J = 8.9 Hz, 1H), 8.14 (d, J = 9.0 Hz, 1H), 7.38-7.19 (t, J = 7.6, 6.4 Hz, 2H), 6.76-6.69 (m, 2H), 4.44-4.33 (m, 1H), 3.73-3.58 (m, 1H), 3.54 (s, 3H), 3.29 (s, 3H), 3.03 (d, J = 5.7 Hz, 2H), 2.11-1.98 (m, 4H), 1.89- 1.75 (m, 2H), 1.69-1.53 (m, 2H), 0.94-0.80 (m, 1H), 0.52-0.42 (m, 2H), 0.24 (q, J = 5.0 Hz, 2H) | 552.24 |
| 77 | 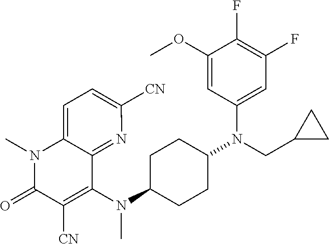 [SMILES: COc1cc(N(CC2CC2)[C@H]2CC[C@H](N(C)c3c(C#N)c(=O)n(C)c4ccc(C#N)nc34)CC2)cc(F)c1F] | 1HNMR (400 MHz, DMSO-d6) δ 8.26 (d, J = 8.9 Hz, 1H), 8.14 (d, J = 9.0 Hz, 1H), 6.43-6.34 (m, 1H), 6.35-6.27 (m, 1H), 4.44-4.32 (m, 1H), 3.87 (s, 3H), 3.72-3.60 (m, 1H), 3.54 (s, 3H), 3.29 (s, 3H), 3.05 (d, J = 5.7 Hz, 2H), 2.16-1.96 (m, 4H), 1.87-1.78 (m, 2H), 1.71-1.53 (m, 2H), 0.97- 0.82 (m, 1H), 0.53-0.44 (m, 2H), 0.28-0.21 (m, 2H) | 533.26 |
| 78 | 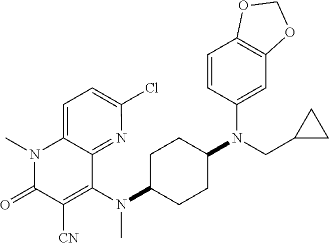 | 1HNMR (400 MHz, DMSO-d6) δ 8.04 (d, J = 9.0 Hz, 1H), 7.78 (d, J = 8.9 Hz, 1H), 6.88-6.79 (m, 2H), 6.68 (dd, J = 8.2, 1.7 Hz, 1H), 5.98 (s, 2H), 4.35 (t, J = 10.5 Hz, 1H), 3.52 (s, 3H), 3.26 (s,3H), 3.29-3.33 (m, 1H), 2.79 (d, J = 6.4 Hz, 2H), 2.22- 2.02 (m, 2H), 1.95-1.80 (m, 2H), 1.72-1.57 (m, 2H), 1.50-1.35 (m, 2H), 0.75-0.59 (m, 1H), 0.39- 0.26 (m, 2H), 0.00-(−)0.10 (m, 2H) | 520.22 |
| 79 | 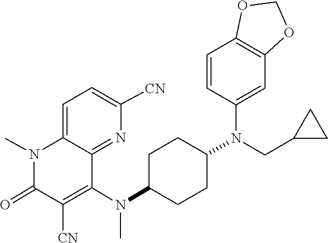 [SMILES: Cn1c(=O)c(C#N)c(N(C)[C@H]2CC[C@H](N(CC3CC3)c3ccc4c(c3)OCO4)CC2)c2nc(C#N)ccc21] | 1HNMR (400 MHz, DMSO-d6) δ 8.25 (d, J = 8.9 Hz, 1H), 8.13 (d, J = 9.0 Hz, 1H), 6.75 (d, J = 8.5 Hz, 1H), 6.60 (d, J = 2.4 Hz, 1H), 6.32 (dd, J = 8.6, 2.4 Hz, 1H), 5.89 (s, 2H), 4.42-4.25 (m, 1H), 3.53 (s, 3H), 3.52-3.43 (m, 1H), 3.27 (s, 3H), 2.94 (d, J = 5.9 Hz, 2H), 2.07-1.95 (m, 4H), 1.89-1.77 (m, 2H), 1.60-1.41 (m, 2H), 0.92-0.78 (m, 1H), 0.49- 0.37 (m, 2H), 0.22-0.10 (m, 2H) | 511.24 |
| 80 | 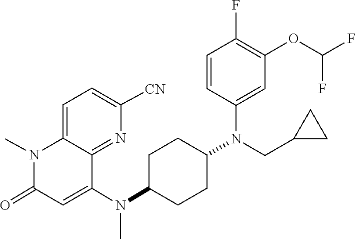 [SMILES: Cn1c(=O)cc(N(C)[C@H]2CC[C@H](N(CC3CC3)c3ccc(F)c(OC(F)F)c3)CC2)c2nc(C#N)ccc21] | 1HNMR (400 MHz, DMSO-d6) δ 7.91 (d, J = 8.8 Hz, 1H), 7.80 (d, J = 8.9 Hz, 1H), 6.95-6.91 (m, 2H), 6.51-6.42 (m, 2H), 5.65 (s, 1H), 4.17-4.03 (m, 1H), 3.46-3.32 (m, 1H), 3.28 (s, 3H), 2.79 (d, J = 5.8 Hz, 2H), 2.65 (s, 3H), 1.73-1.63 (m, 4H), 1.59- 1.50 (m, 2H), 1.40-1.18 (m, 2H), 0.71-056 (m, 1H), 0.31-0.18 (m, 2H), 0.04-−0.07 (m, 2H) | 526.26 |
| 81 | 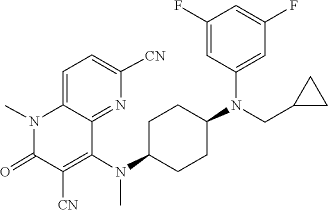 [SMILES: Cn1c(=O)c(C#N)c(N(C)[C@H]2CC[C@@H](N(CC3CC3)c3cc(F)cc(F)c3)CC2)c2nc(C#N)ccc21] | 1HNMR (400 MHz, DMSO-d6) δ 8.29 (d, J = 8.9 Hz, 1H), 8.20 (d, J = 9.0 Hz, 1H), 6.51 (dd, J = 11.7, 2.0 Hz, 2H), 6.40-6.32 (m, 1H), 4.47-4.36 (m, 1H), 3.79-3.66 (m, 1H), 3.58 (s, 3H), 3.24 (s, 3H), 3.08 (d, J = 5.7 Hz, 2H), 2.17-2.04 (m, 2H), 1.87- 1.73 (m, 2H), 1.71-1.58 (m, 2H), 1.57-1.47 (m, 2H), 0.89-0.77 (m, 1H), 0.46-0.36 (m, 2H), 0.19- 0.10 (m, 2H) | 503.25 |
| 82 | 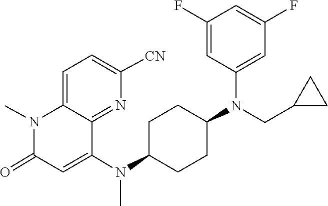 [SMILES: Cn1c(=O)cc(N(C)[C@H]2CC[C@@H](N(CC3CC3)c3cc(F)cc(F)c3)CC2)c2nc(C#N)ccc21] | 1HNMR (400 MHz, DMSO-d6) δ 7.85 (d, J = 8.8 Hz, 1H), 7.80 (d, J = 8.8 Hz, 1H), 6.37-6.29 (m, 2H), 6.14 (tt, J = 9.1, 2.2 Hz, 1H), 5.89 (s, 1H), 4.04-3.94 (m, 1H), 3.56-3.45 (m, 1H), 3.34 (d, J = 6.5 Hz, 3H), 2.96 (d, J = 5.8 Hz, 2H), 2.77 (s, 3H), 1.89 (dtd, J = 11.7, 7.4, 4.1 Hz, 2H), 1.70 (dtd, J = 12.3, 8.2, 3.9 Hz, 2H), 1.53 (ddd, J = 12.8, 8.3, 4.0 Hz, 2H), 1.40 (ddd, J = 16.9, 8.0, 4.1 Hz, 2H), 0.72-0.61 (m, 1H), 0.31-0.23 (m, 2H), 0.04-(−)0.04 (m, 2H) | 478.25 |
| 83 | 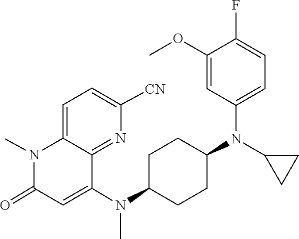 [SMILES: COc1cc(N(C2CC2)[C@H]2CC[C@@H](N(C)c3cc(=O)n(C)c4ccc(C#N)nc34)CC2)ccc1F] | 1HNMR (400 MHz, DMSO-d6) δ 8.14 (d, J = 8.8 Hz, 1H), 8.03 (d, J = 8.9 Hz, 1H), 7.08 (dd, J = 11.5, 8.6 Hz, 1H), 6.93 (dd, J = 8.0, 2.2 Hz, 1H), 6.69 (ddd, J = 8.5, 8.0, 2.5 Hz, 1H), 5.92 (s, 1H), 4.33-4.23 (m, 1H), 3.82 (s, 3H), 3.51 (s, 3H), 2.89 (s, 3H), 2.33-2.26 (m, 1H), 2.08-1.94 (m, 4H), 1.60-1.52 (m, 2H), 1.50-1.42 (m, 2H), 1.22 (dd, J = 9.2, 4.4 Hz, 1H), 0.61-0.53 (m, 2H), 0.46-0.39 (m, 2H) | 476.26 |
| 84 | 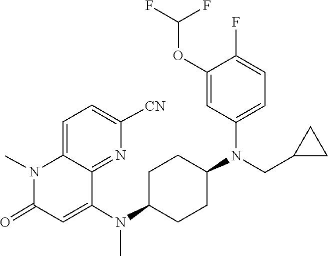 [SMILES: Cn1c(=O)cc(N(C)[C@H]2CC[C@@H](N(CC3CC3)c3ccc(F)c(OC(F)F)c3)CC2)c2nc(C#N)ccc21] | 1HNMR (400 MHz, DMSO-d6) δ 8.14 (d, J = 8.8 Hz, 1H), 8.04 (d, J = 8.9 Hz, 1H), 7.28 (dd, J = 10.4, 9.0 Hz, 1H), 7.23 (t, J = 74.0 Hz, 1H), 7.12- 7.02 (m, 2H), 5.95 (s, 1H), 4.34-4.21 (m, 1H), 3.51 (s, 3H), 3.49-3.42 (m, 1H), 2.94 (d, J = 6.4 Hz, 2H), 2.89 (s, 3H), 2.07-1.93 (m, 2H), 1.93-1.83 (m, 2H), 1.63-1.51 (m, 2H), 1.51-1.37 (m, 2H), 0.79- 0.66 (m, 1H), 0.38-0.27 (m, 2H), 0.00-(−)0.11 (m, 2H) | 526.26 |
| 85 | 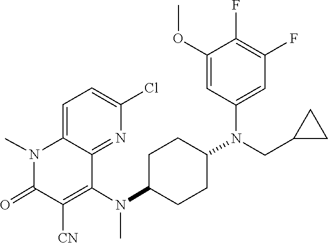 [SMILES: COc1cc(N(CC2CC2)[C@H]2CC[C@H](N(C)c3c(C#N)c(=O)n(C)c4ccc(Cl)nc34)CC2)cc(F)c1F] | 1HNMR (400 MHz, DMSO-d6) δ 8.05 (d, J = 9.0 Hz, 1H), 7.80 (d, J = 9.0 Hz, 1H), 6.42-6.28 (m, 2H), 4.42-4.24 (m, 1H), 3.86 (s, 3H), 3.65 (m, J = 10.8 Hz, 1H), 3.52 (s, 3H), 3.26 (s, 3H), 3.04 (d, J = 5.7 Hz, 2H), 2.05 (m, J = 7.1 Hz, 4H), 1.82 (d, J = 11.2 Hz, 2H), 1.62 (m, J = 17.6, 10.4 Hz, 2H), 0.89 (m, J = 13.4, 6.6, 4.1 Hz, 1H), 0.54-0.43 (m, 2H), 0.28-0.19 (m, 2H) | 542.23 |
| 86 | 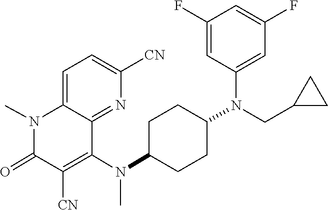 [SMILES: Cn1c(=O)c(C#N)c(N(C)[C@H]2CC[C@H](N(CC3CC3)c3cc(F)cc(F)c3)CC2)c2nc(C#N)ccc21] | 1HNMR (400 MHz, DMSO-d6) δ 8.27 (d, J = 8.9 Hz, 1H), 8.14 (d, J = 8.9 Hz, 1H), 6.52-6.43 (m, 2H), 6.35-6.27 (m, 1H), 4.45-4.35 (m, 1H), 3.80- 3.69 (m, 1H), 3.54 (s, 3H), 3.29 (s, 3H), 3.07 (d, J = 5.6 Hz, 2H), 2.21-1.98 (m, 4H), 1.87-1.76 (m, 2H), 1.71-1.56 (m, 2H), 0.93-0.84 (m, 1H), 0.54- 0.43 (m, 2H), 0.32-0.22 (m, 2H) | 503.24 |
| 87 | 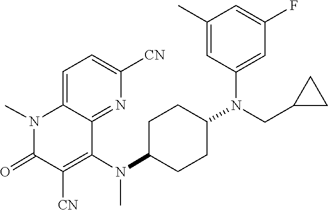 [SMILES: Cc1cc(F)cc(N(CC2CC2)[C@H]2CC[C@H](N(C)c3c(C#N)c(=O)n(C)c4ccc(C#N)nc34)CC2)c1] | 1HNMR (400 MHz, DMSO-d6) δ 8.26 (d, J = 8.9 Hz, 1H), 8.14 (d, J = 8.9 Hz, 1H), 6.48 (s, 1H), 6.41 (d, J = 13.6 Hz, 1H), 6.22 (d, J = 9.2 Hz, 1H), 4.45-4.34 (m, 1H), 3.77-3.63 (m, 1H), 3.54 (s, 3H), 3.30 (s, 3H), 3.04 (d, J = 5.6 Hz, 2H), 2.26 (s, 3H), 2.15-2.01 (m, 4H), 1.85-1.77 (m, 2H), 1.69-1.56 (m, 2H), 0.96-0.85 (m, 1H), 0.51-0.45 (m, 2H), 0.29-0.22 (m, 2H) | 499.28 |
| 88 | 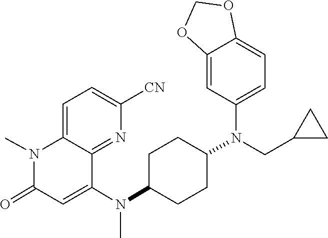 [SMILES: Cn1c(=O)cc(N(C)[C@H]2CC[C@H](N(CC3CC3)c3ccc4c(c3)OCO4)CC2)c2nc(C#N)ccc21] | 1HNMR (400 MHz, DMSO-d6) δ 8.14 (d, J = 8.8 Hz, 1H), 8.03 (d, J = 8.9 Hz, 1H), 6.75 (d, J = 8.5 Hz, 1H), 6.59 (d, J = 2.3 Hz, 1H), 6.31 (dd, J = 8.6, 2.4 Hz, 1H), 5.89-5.88 (s, 3H), 4.38-4.24 (m, 1H), 3.51 (s, 3H), 3.49-3.39 (m, 1H), 2.94 (d, J = 5.8 Hz, 2H), 2.88 (s, 3H), 1.97-1.73 (m, 6H), 1.58-1.39 (m, 2H), 0.92-0.79 (m, 1H), 0.48-0.36 (m, 2H), 0.21-0.12 (m, 2H) | 487.26 |
| 89 | 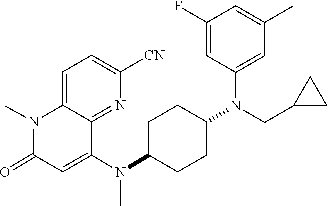 [SMILES: Cc1cc(F)cc(N(CC2CC2)[C@H]2CC[C@H](N(C)c3cc(=O)n(C)c4ccc(C#N)nc34)CC2)c1] | 1HNMR (400 MHz, DMSO-d6) δ 8.14 (d, J = 8.8 Hz, 1H), 8.03 (d, J = 8.8 Hz, 1H), 6.46 (s, 1H), 6.39 (d, J = 13.6 Hz, 1H), 6.20 (d, J = 9.1 Hz, 1H), 5.88 (s, 1H), 4.43-4.27 (m, 1H), 3.67 (t, J = 11.0 Hz, 1H), 3.51 (s, 3H), 3.03 (d, J = 5.3 Hz, 2H), 2.89 (s, 3H), 2.25 (s, 3H), 2.01-1.85 (m, 4H), 1.84- 1.70 (m, 2H), 1.65-1.47 (m, 2H), 0.97-0.82 (m, 1H), 0.48 (d, J = 7.5 Hz, 2H), 0.24 (d, J = 4.3 Hz, 2H) | 475.27 |
| 90 | 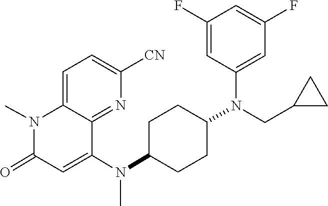 [SMILES: Cn1c(=O)cc(N(C)[C@H]2CC[C@H](N(CC3CC3)c3cc(F)cc(F)c3)CC2)c2nc(C#N)ccc21] | 1HNMR (400 MHz, DMSO-d6) δ 8.15 (d, J = 8.8 Hz, 1H), 8.04 (d, J = 8.8 Hz, 1H), 6.46 (d, J = 10.8 Hz, 2H), 6.30 (t, J = 8.6 Hz, 1H), 5.89 (s, 1H), 4.42-4.27 (m, 1H), 3.78-3.64 (m, 1H), 3.51 (s, 3H), 3.07 (d, J = 5.2 Hz, 2H), 2.89 (s, 3H), 2.04-1.87 (m, 4H), 1.82-1.73 (m, 2H), 1.67-1.53 (m, 2H), 0.94-0.82 (m, 1H), 0.54-0.44 (m, 2H), 0.30-0.23 (m, 2H) | 478.26 |
| 91 | 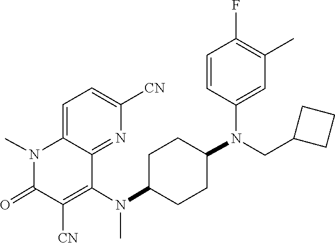 [SMILES: Cc1cc(N(CC2CCC2)[C@H]2CC[C@@H](N(C)c3c(C#N)c(=O)n(C)c4ccc(C#N)nc34)CC2)ccc1F] | 1HNMR (400 MHz, DMSO-d6) δ 8.25 (d, J = 8.9 Hz, 1H), 8.13 (d, J = 8.9 Hz, 1H), 7.09-6.99 (m, 2H), 6.99-6.92 (m, 1H), 4.47-4.28 (m, 1H), 3.53 (s, 3H), 3.29 (s, 3H), 3.24-3.19 (m, 1H), 2.94 (d, J = 7.1 Hz, 2H), 2.21 (s, 3H), 2.19-2.08 (m, 3H), 1.89- 1.79 (m, 4H), 1.78-1.50 (m, 6H), 1.49-1.36 (m, 2H) | 513.30 |
| 92 | 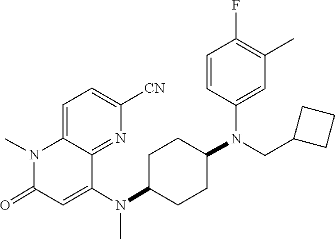 [SMILES: Cc1cc(N(CC2CCC2)[C@H]2CC[C@@H](N(C)c3cc(=O)n(C)c4ccc(C#N)nc34)CC2)ccc1F] | 1HNMR (400 MHz, DMSO-d6) δ 8.13 (d, J = 8.7 Hz, 1H), 8.03 (d, J = 8.9 Hz, 1H), 7.12-6.93 (m, 3H), 5.91 (s, 1H), 4.39-4.24 (m, 1H), 3.51 (s, 3H), 2.93 (d, J = 7.1 Hz, 2H), 2.89 (s, 3H), 2.21 (s, 3H), 2.17-1.93 (m, 4H), 1.91-1.66 (m, 6H), 1.63-1.46 (m, 4H), 1.38 (t, J = 12.5 Hz, 2H) | 489.28 |
| 93 | 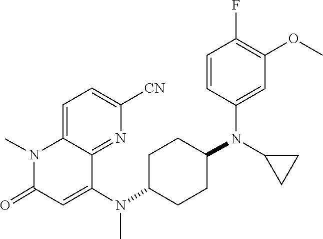 | 1HNMR (400 MHz, DMSO-d6) δ 8.15 (d, J = 8.8 Hz, 1H), 8.04 (d, J = 8.9 Hz, 1H), 7.00 (dd, J = 11.4, 8.9 Hz, 1H), 6.75 (dd, J = 7.8, 2.7 Hz, 1H), 6.54 (dt, J = 8.8, 3.3 Hz, 1H), 5.87 (s, 1H), 4.37 (t, J = 10.5 Hz, 1H), 3.81 (s, 3H), 3.51 (s, 3H), 3.47 (t, J = 10.9 Hz, 1H), 2.85 (s, 3H), 2.35-2.27 (m, 1H), 1.94-1.84 (m, 4H), 1.82-1.66 (m, 4H), 0.79-0.72 (m, 2H), 0.37-0.30 (m, 2H) | 476.26 |
| 94 | 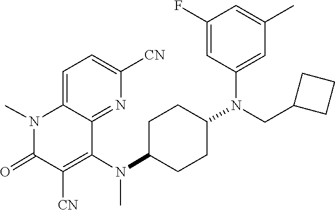 [SMILES: Cc1cc(F)cc(N(CC2CCC2)[C@H]2CC[C@H](N(C)c3c(C#N)c(=O)n(C)c4ccc(C#N)nc34)CC2)c1] | 1HNMR (400 MHz, DMSO-d6) δ 8.26 (d, J = 8.9 Hz, 1H), 8.18-8.08 (m, 1H), 6.36 (s, 1H), 6.29 (d, J = 13.5 Hz, 1H), 6.21 (d, J = 9.1 Hz, 1H), 4.46-4.33 (m, 1H), 3.67 (t, J = 11.4 Hz, 1H), 3.54 (s, 3H), 3.29 (s, 3H), 3.12 (d, J = 6.1 Hz, 2H), 2.41 (dd, J = 13.2, 6.4 Hz, 1H), 2.23 (s, 3H), 2.12-1.92 (m, 6H), 1.81-1.55 (m, 8H) | 513.29 |
| 95 | 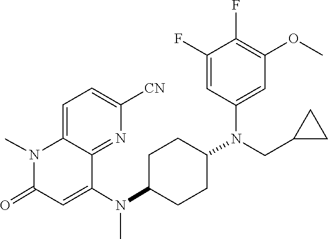 [SMILES: COc1cc(N(CC2CC2)[C@H]2CC[C@H](N(C)c3cc(=O)n(C)c4ccc(C#N)nc34)CC2)cc(F)c1F] | 1HNMR (400 MHz, DMSO-d6) δ 8.15 (d, J = 8.8 Hz, 1H), 8.04 (d, J = 8.9 Hz, 1H), 6.40-6.28 (m, 2H), 5.89 (s, 1H), 4.40-4.28 (m, 1H), 3.86 (s, 3H), 3.70-3.58 (m, 1H), 3.52 (s, 3H), 3.05 (d, J = 5.7 Hz, 2H), 2.89 (s, 3H), 2.00-1.87 (m, 4H), 1.79 (d, J = 11.7 Hz, 2H), 1.64-1.50 (m, 2H), 0.95-0.83 (m, 1H), 0.53-0.45 (m, 2H), 0.28-0.21 (m, 2H) | 508.27 |
| 96 | 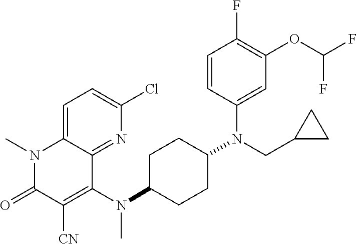 [SMILES: Cn1c(=O)c(C#N)c(N(C)[C@H]2CC[C@H](N(CC3CC3)c3ccc(F)c(OC(F)F)c3)CC2)c2nc(Cl)ccc21] | 1HNMR (400 MHz, DMSO-d6) δ 8.05 (d, J = 9.0 Hz, 1H), 7.80 (d, J = 8.9 Hz, 1H), 7.20 (t, J = 73.8 Hz, 1H), 7.23-7.14 (m, 1H), 6.76-6.67 (m, 2H), 4.39-4.26 (m, 1H), 3.70-3.58 (m, 1H), 3.52 (s, 3H), 3.25 (s, 3H), 3.03 (d, J = 5.7 Hz, 2H), 2.12-1.96 (m, 4H), 1.82 (d, J = 11.5 Hz, 2H), 1.68-1.51 (m, 2H), 0.92-0.82 (m, 1H), 0.51-0.43 (m, 2H), 0.26- 0.19 (m, 2H) | 560.22 |
| 97 | 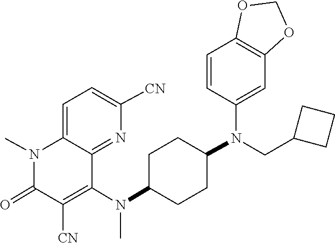 [SMILES: Cn1c(=O)c(C#N)c(N(C)[C@H]2CC[C@@H](N(CC3CCC3)c3ccc4c(c3)OCO4)CC2)c2nc(C#N)ccc21] | 1HNMR (400 MHz, DMSO-d6) δ 8.24 (d, J = 8.9 Hz, 1H), 8.12 (d, J = 8.9 Hz, 1H), 6.82 (dd, J = 5.0, 3.1 Hz, 2H), 6.63 (dd, J = 8.2, 1.8 Hz, 1H), 5.98 (s, 2H), 4.44-4.30 (m, 1H), 3.53 (s, 3H), 3.29 (s, 3H), 3.09 (bs, 1H), 2.86 (d, J = 7.3 Hz, 2H), 2.25-2.05 (m, 3H), 1.92-1.79 (m, 4H), 1.79-1.67 (m, 2H), 1.67-1.49 (m, 4H), 1.50-1.31 (m, 2H) | 525.28 |
| 98 | 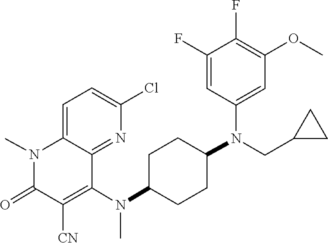 [SMILES: COc1cc(N(CC2CC2)[C@H]2CC[C@@H](N(C)c3c(C#N)c(=O)n(C)c4ccc(Cl)nc34)CC2)cc(F)c1F] | 1HNMR (400 MHz, DMSO-d6) δ 8.08 (d, J = 9.0 Hz, 1H), 7.81 (d, J = 9.0 Hz, 1H), 6.63-6.54 (m, 2H), 4.42-4.32 (m, 1H), 3.85 (s, 3H), 3.55-3.50 (s, 4H), 3.23 (s, 3H), 2.99 (d, J = 6.2 Hz, 2H), 2.14- 2.02 (m, 2H), 1.82-1.66 (m, 4H), 1.54-1.41 (m, 2H), 0.82-0.72 (m, 1H), 0.41-0.31 (m, 2H), 0.03 (q, J = 5.4 Hz, 2H) | 542.22 |
| 99 | 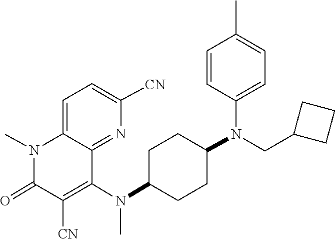 [SMILES: Cc1ccc(N(CC2CCC2)[C@H]2CC[C@@H](N(C)c3c(C#N)c(=O)n(C)c4ccc(C#N)nc34)CC2)cc1] | 1HNMR (400 MHz, DMSO-d6) δ 8.25 (d, J = 8.8 Hz, 1H), 8.14 (d, J = 8.9 Hz, 1H), 7.08 (d, J = 8.1 Hz, 2H), 7.00 (d, J = 8.1 Hz, 2H), 4.43-4.30 (m, 1H), 3.53 (s, 3H), 3.28 (s, 3H), 3.26-3.22 (m, 1H), 2.95 (d, J = 7.1 Hz, 2H), 2.25 (s, 3H), 2.21-2.09 (m, 3H), 1.89-1.77 (m, 4H), 1.76-1.68 (m, 2H), 1.68-1.60 (m, 2H), 1.59-1.50 (m, 2H), 1.48-1.36 (m, 2H) | 495.28 |
| 100 | 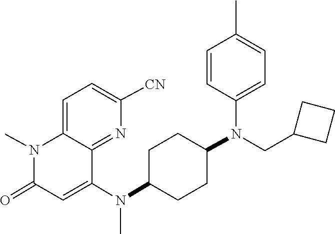 [SMILES: Cc1ccc(N(CC2CCC2)[C@H]2CC[C@@H](N(C)c3cc(=O)n(C)c4ccc(C#N)nc34)CC2)cc1] | 1HNMR (400 MHz, DMSO-d6) δ 8.13 (d, J = 8.8 Hz, 1H), 8.03 (d, J = 8.9 Hz, 1H), 7.09 (d, J = 8.2 Hz, 2H), 7.02 (d, J = 8.3 Hz, 2H), 5.91 (s, 1H), 4.36-4.25 (m, 1H), 3.51 (s, 3H), 3.22 (s, 1H), 2.94 (d, J = 7.1 Hz, 2H), 2.88 (s, 3H), 2.25 (s, 3H), 2.19- 1.98 (m, 3H), 1.90-1.77 (m, 4H), 1.77-1.66 (m, 2H), 1.62-1.44 (m, 4H), 1.43-1.31 (m, 2H) | 471.29 |
| 101 | 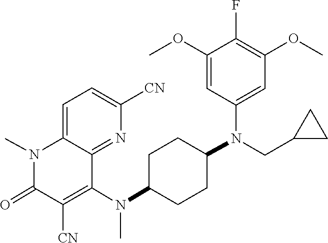 [SMILES: COc1cc(N(CC2CC2)[C@H]2CC[C@@H](N(C)c3c(C#N)c(=O)n(C)c4ccc(C#N)nc34)CC2)cc(OC)c1F] | 1HNMR (400 MHz, DMSO-d6) δ 8.26 (d, J = 8.9 Hz, 1H), 8.15 (d, J = 9.0 Hz, 1H), 6.51 (d, J = 6.8 Hz, 2H), 4.44-4.35 (m, 1H), 3.80 (s, 6H), 3.54 (s, 3H), 3.49-3.43 (m, 1H), 3.29 (s, 3H), 2.91 (d, J = 6.3 Hz, 2H), 2.22-2.08 (m, 2H), 1.92-1.82 (m, 2H), 1.77-1.62 (m, 2H), 1.55-1.40 (m, 2H), 0.79-0.69 (m, 1H), 0.39-0.32 (m, 2H), 0.02-(−)0.04 (m, 2H) | 545.28 |
| 102 | 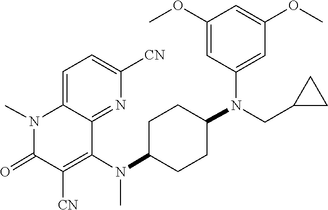 [SMILES: COc1cc(OC)cc(N(CC2CC2)[C@H]2CC[C@@H](N(C)c3c(C#N)c(=O)n(C)c4ccc(C#N)nc34)CC2)c1] | 1HNMR (400 MHz, DMSO-d6) δ 8.26 (d, J = 8.9 Hz, 1H), 8.14 (d, J = 9.0 Hz, 1H), 5.93 (d, J = 1.9 Hz, 1H), 5.83 (d, J = 1.7 Hz, 1H), 4.45-4.34 (m, 1H), 4.31 (d, J = 5.4 Hz, 1H), 3.71 (s, 3H), 3.70(s, 3H), 3.66-3.59 (m, 1H), 3.27 (s, 3H), 2.57 (d, J = 6.0 Hz, 2H), 2.13-1.94 (m, 4H), 1.92-1.77 (m, 2H), 1.75-1.61 (m, 2H), 0.99-0.86 (m, 1H), 0.43-0.32 (m, 2H), 0.22-0.13 (m, 2H) | 526.29 |
| 103 | 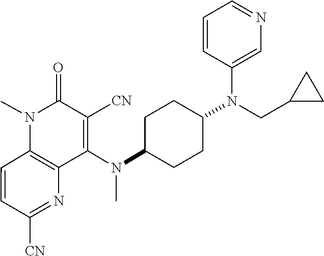 [SMILES: Cn1c(=O)c(C#N)c(N(C)[C@H]2CC[C@H](N(CC3CC3)c3cccnc3)CC2)c2nc(C#N)ccc21] | 1H NMR (300 MHz, DMSO-d6) δ = 8.35-8.25 (m, 2H), 8.16 (d, J = 9.0 Hz, 1H), 8.04 (d, J = 5.0 Hz, 1H), 7.85 (d, J = 8.8 Hz, 1H), 7.70 (dd, J = 5.1, 8.7 Hz, 1H), 4.48-4.30 (m, 1H), 3.98-3.71 (m, 1H), 3.55 (s, 3H), 3.30 (s, 3H), 3.23 (s, 3H), 2.15 (br s, 1H), 2.19-2.00 (m, 4H), 1.96-1.80 (m, 2H), 1.80- 1.48 (m, 2H), 0.99-0.79 (m, 1H), 0.52 (d, J = 7.8 Hz, 2H), 0.33 (d, J = 4.9 Hz, 2H) | 468.5 |
| 104 | 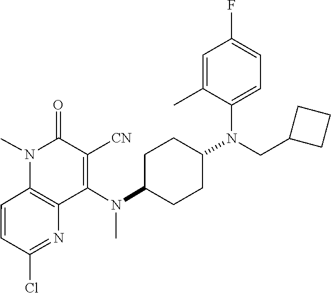 [SMILES: Cc1cc(F)ccc1N(CC1CCC1)[C@H]1CC[C@H](N(C)c2c(C#N)c(=O)n(C)c3ccc(Cl)nc23)CC1] | 1H NMR (300 MHz, DMSO-d6) δ = 8.05 (d, J = 8.9 Hz, 1H), 7.80 (d, J = 8.6 Hz, 1H), 7.22-7.14 (m, 1H), 7.02 (d, J = 9.9 Hz, 1H), 6.93 (t, J = 8.5 Hz, 1H), 4.30-4.17 (m, 1H), 3.52 (s, 3H), 3.18 (s, 3H), 2.98 (d, J = 7.0 Hz, 2H), 2.66-2.56 (m, 1H), 2.22 (s, 3H), 2.09-1.93 (m, 3H), 1.92-1.64 (m, 8H), 1.61-1.39 (m, 4H) | 522.5 |
| 105 | 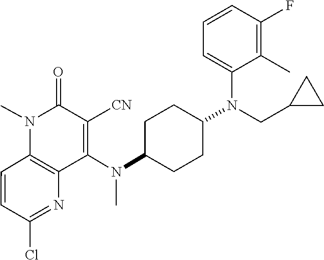 [SMILES: Cc1c(F)cccc1N(CC1CC1)[C@H]1CC[C@H](N(C)c2c(C#N)c(=O)n(C)c3ccc(Cl)nc23)CC1] | 1H NMR (300 MHz, DMSO-d6) δ = 8.05 (d, J = 9.0 Hz, 1H), 7.80 (d, J = 8.9 Hz, 1H), 7.21-7.04 (m, 2H), 6.87 (t, J = 8.6 Hz, 1H), 4.29-4.17 (m, 1H), 3.52 (s, 3H), 3.26 (s, 1H), 3.18 (s, 3H), 2.92-2.72 (m, 3H), 2.47-2.24 (m, 1H), 2.20 (s, 3H), 1.99 (d, J = 10.5 Hz, 2H), 1.93-1.73 (m, 4H), 1.60-1.35 (m, 2H), 0.69-0.57 (m, 1H), 0.26 (d, J = 7.5 Hz, 2H), −0.02 (d, J = 4.7 Hz, 2H) | 508.1 |
| 106 | 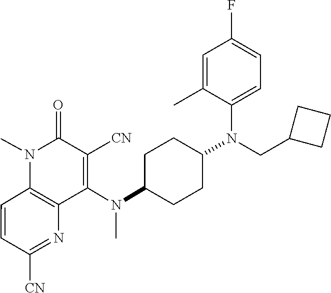 [SMILES: Cc1cc(F)ccc1N(CC1CCC1)[C@H]1CC[C@H](N(C)c2c(C#N)c(=O)n(C)c3ccc(C#N)nc23)CC1] | 1H NMR (300 MHz, DMSO-d6) δ = 8.24 (d, J = 8.8 Hz, 1H), 8.12 (d, J = 8.9 Hz, 1H), 7.25-7.08 (m, 1H), 7.08-6.85 (m, 2H), 4.42-4.10 (m, 1H), 3.63-3.42 (m, 3H), 3.20 (s, 3H), 2.97 (d, J = 6.6 Hz, 2H), 2.71-2.54 (m, 1H), 2.22 (s, 3H), 2.11- 1.90 (m, 4H), 1.90-1.64 (m, 7H), 1.60-1.36 (m, 4H) | 514.1 |
| 107 | 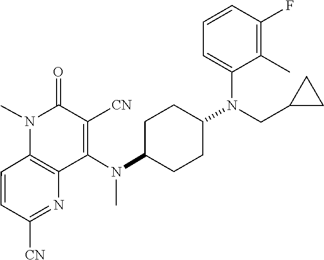 [SMILES: Cc1c(F)cccc1N(CC1CC1)[C@H]1CC[C@H](N(C)c2c(C#N)c(=O)n(C)c3ccc(C#N)nc23)CC1] | 1H NMR (300 MHz, DMSO-d6) δ = 8.26 (d, J = 8.5 Hz, 1H), 8.14 (d, J = 8.6 Hz, 1H), 7.23-7.04 (m, 2H), 6.87 (t, J = 8.5 Hz, 1H), 4.36-4.20 (m, 1H), 3.53 (s, 3H), 3.22 (s, 2H), 2.88 (d, J = 6.4 Hz, 1H), 2.20 (s, 3H), 2.06-1.75 (m, 6H), 1.56-1.38 (m, 2H), 0.70-0.55 (m, 1H), 0.27 (d, J = 7.8 Hz, 2H), −0.01 (d, J = 4.5 Hz, 2H) | 500.4 |
| 108 | 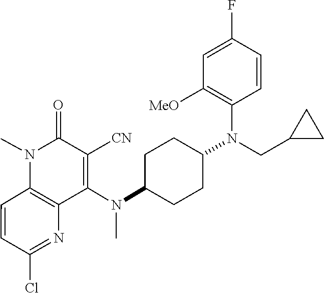 [SMILES: COc1cc(F)ccc1N(CC1CC1)[C@H]1CC[C@H](N(C)c2c(C#N)c(=O)n(C)c3ccc(Cl)nc23)CC1] | (300 MHz, CDCl3) δ 7.68-7.58 (m, 1H), 7.56-7.47 (m, 1H), 7.08 (bt, J = 7.0 Hz, 1H), 6.84-6.74 (m, 1H), 6.66-6.54 (m, 1H), 4.43-4.29 (m, 1H), 3.84 (s, 3H), 3.64 (m, 3H), 3.33 (s, 3H), 3.13-2.99 (m, 1H), 2.94 (d, J = 6.3 Hz, 2H), 2.47-1.97 (m, 4H), 1.90- 1.72 (m, 2H), 1.54-1.21 (m, 2H), 0.94-0.70 (m, 1H), 0.33-0.27 (m, 2H), 0-(−)0.08 (m, 2H) | 524.46 |
| 109 | 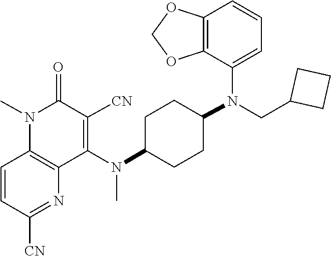 [SMILES: Cn1c(=O)c(C#N)c(N(C)[C@H]2CC[C@@H](N(CC3CCC3)c3cccc4c3OCO4)CC2)c2nc(C#N)ccc21] | (300 MHz, CDCl3) δ 7.84 (d, J = 8.8 Hz, 1H), 7.70 (d, J = 8.5 Hz, 1H), 6.80-6.72 (m, 1H), 6.72-6.62 (m, 2H), 5.94 (s, 2H), 4.57-4.34 (m, 1H), 3.63 (s, 3H), 3.43 (s, 3H), 3.37 (br s, 1H), 2.99 (d, J = 7.3 Hz, 2H), 2.41-2.15 (m, 3H), 2.10-1.97 (m, 2H), 1.97-1.84 (m, 2H), 1.83-1.68 (m, 4H), 1.58-1.39 (m, 4H) | |
| 110 | 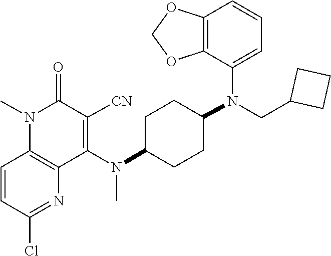 [SMILES: Cn1c(=O)c(C#N)c(N(C)[C@H]2CC[C@@H](N(CC3CCC3)c3cccc4c3OCO4)CC2)c2nc(Cl)ccc21] | (300 MHz, CDCl3) δ 7.61 (d, J = 8.7 Hz, 1H), 7.48 (d, J = 8.9 Hz, 1H), 6.81-6.72 (m, 1H), 6.72-6.62 (m, 2H), 5.94 (s, 2H), 4.56-4.40 (m, 1H), 3.61 (s, 3H), 3.40 (s, 3H), 3.37 (br s, 1H), 3.00 (d, J = 7.2 Hz, 2H), 2.42-2.13 (m, 3H), 2.02 (bd, J = 13.9 Hz, 2H), 1.95-1.67 (m, 6H), 1.65-1.41 (m, 4H) | 534.93 |
| 111 | 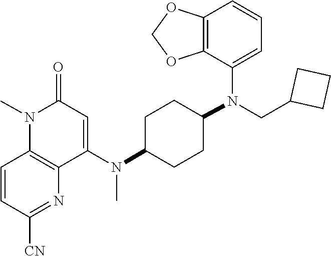 [SMILES: Cn1c(=O)cc(N(C)[C@H]2CC[C@@H](N(CC3CCC3)c3cccc4c3OCO4)CC2)c2nc(C#N)ccc21] | (300 MHz, CDCl3) δ ppm 7.78 (d, J = 8.6 Hz, 1H), 7.67 (d, J = 8.8 Hz, 1H), 6.82-6.73 (m, 1H), 6.73- 6.64 (m, 2H), 6.03 (s, 1H), 5.95 (s, 2H), 4.52-4.36 (m, 1H), 3.63 (s, 3H), 3.39 (br s, 1H), 3.07-2.96 (m, 5H), 2.36 (dt, J = 14.6, 7.4 Hz, 1H), 2.22-1.97 (m, 4 H), 1.97-1.69 (m, 4 H), 1.67-1.38 (m, 6H) | 500.98 |
| 112 | 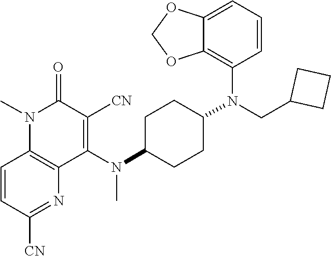 [SMILES: Cn1c(=O)c(C#N)c(N(C)[C@H]2CC[C@H](N(CC3CCC3)c3cccc4c3OCO4)CC2)c2nc(C#N)ccc21] | (300 MHz, CDCl₃) δ ppm 7.86 (d, J = 8.6 Hz, 1H), 7.72 (d, J = 8.8 Hz, 1H), 6.78-6.66 (m, 1 H), 6.48 (br t, J = 6.7 Hz, 2H), 5.93 (s, 2 H), 4.43-4.30 (m, 1 H), 3.64 (s, 3H), 3.37 (s, 3H), 3.35-3.27 (m, 1 H), 3.10 (bd, J = 6.8 Hz, 2H), 2.47-2.32 (m, 1H), 2.16 (bd, J = 11.8 Hz, 2H), 2.03 (bd, J = 12.8 Hz, 2H), 1.97-1.72 (m, 7 H), 1.69-1.60 (m, 3H). | 525.92 |
| 113 | 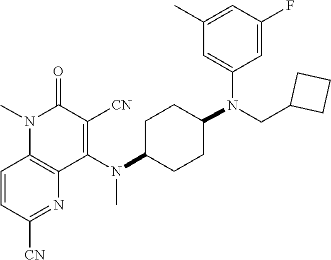 [SMILES: Cc1cc(F)cc(N(CC2CCC2)[C@H]2CC[C@@H](N(C)c3c(C#N)c(=O)n(C)c4ccc(C#N)nc34)CC2)c1] | (400 MHz, DMSO) δ 8.28 (d, J = 8.9 Hz, 1H), 8.18 (d, J = 9.0 Hz, 1H), 6.53 (s, 1H), 6.47 (d, J = 12.5 Hz, 1H), 6.39 (d, J = 9.3 Hz, 1H), 4.43-4.34 (m, 1H), 3.56 (s, 3H), 3.55-3.48 (m, 1H), 3.27 (s, 3H), 3.09 (d, J = 6.6 Hz, 2H), 2.36-2.26 (m, 1H), 2.24 (s, 3H), 2.17-2.06 (m, 2H), 1.90-1.79 (m, 2H), 1.78-1.63 (m, 6H), 1.63-1.51 (m, 2H), 1.50-1.39 (m, 2H) | 513.30 |
| 114 | 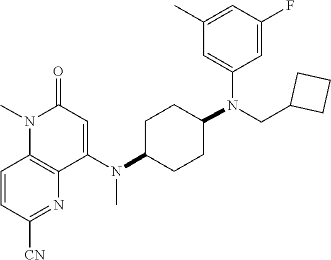 [SMILES: Cc1cc(F)cc(N(CC2CCC2)[C@H]2CC[C@@H](N(C)c3cc(=O)n(C)c4ccc(C#N)nc34)CC2)c1] | (400 MHz, DMSO) δ 8.05 (d, J = 8.8 Hz, 1H), 8.00 (d, J = 8.8 Hz, 1H), 6.61 (s, 1H), 6.51 (d, J = 12.2 Hz, 1H), 6.44 (d, J = 9.4 Hz, 1H), 6.03 (s, 1H), 4.32-4.21 (m, 1H), 3.54 (s, 3H), 3.50 (s, 1H), 3.16 (d, J = 6.6 Hz, 2H), 2.95 (s, 3H), 2.38 (dt, J = 14.9, 7.4 Hz, 1H), 2.27 (s, 3H), 2.13-2.02 (m, 2H), 1.98- 1.84 (m, 4H), 1.81-1.71 (m, 2H), 1.70-1.60 (m, 4H), 1.57-1.47 (m, 2H) | 488.31 |
| 115 | 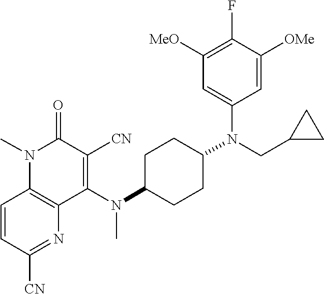 [SMILES: COc1cc(N(CC2CC2)[C@H]2CC[C@H](N(C)c3c(C#N)c(=O)n(C)c4ccc(C#N)nc34)CC2)cc(OC)c1F] | (400 MHz, DMSO-d6) δ ppm: 8.26 (d, J = 8.9 Hz, 1H), 8.14 (d, J = 8.9 Hz, 1H), 6.16 (d, J = 6.4 Hz, 2H), 4.42-4.32 (m, 1H), 3.81 (s, 6H), 3.69-3.58 (m, 1H), 3.54 (s, 3H), 3.29 (s, 3H), 3.03 (d, J = 5.7 Hz, 2H), 2.11-1.96 (m, 4H), 1.91-1.82 (m, 2H), 1.68- 1.52 (m, 2H), 0.97-0.82 (m, 1H), 0.51-0.45 (m, 2H), 0.26-0.20 (m, 2H) | 545.27 |
| 116 | 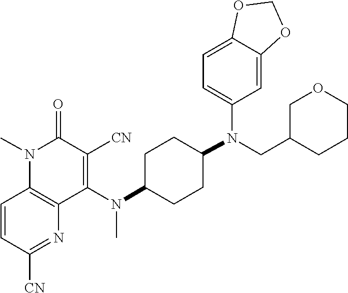 [SMILES: Cn1c(=O)c(C#N)c(N(C)[C@H]2CC[C@@H](N(CC3CCCOC3)c3ccc4c(c3)OCO4)CC2)c2nc(C#N)ccc21] | (400 MHz, DMSO) δ 8.28-8.20 (m, 1H), 8.13 (d, J = 8.9 Hz, 1H), 6.93 (d, J = 1.8 Hz, 1H), 6.86 (d, J = 8.2 Hz, 1H), 6.73 (dd, J = 8.2, 1.8 Hz, 1H), 6.01 (s, 2H), 4.45-4.27 (m, 1H), 4.01-3.86 (m, 1H), 3.74- 3.61 (m, 1H), 3.53 (s, 3H), 3.31 (s, 3H), 3.29-3.24 (m, 1H), 3.17-3.07 (m, 1H), 3.00 (s, 1H), 2.82-2.71 (m, 1H), 2.68-2.58 (m, 1H), 2.25-2.09 (m, 2H), 2.01-1.83 (m, 1H), 1.79-1.58 (m, 4H), 1.56-1.46 (m, 1H), 1.46-1.28 (m, 4H), 1.26-1.11 (m, 1H) | 555.29 |
| 117 | 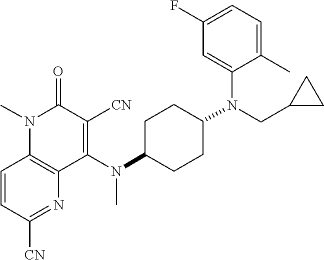 [SMILES: Cc1ccc(F)cc1N(CC1CC1)[C@H]1CC[C@H](N(C)c2c(C#N)c(=O)n(C)c3ccc(C#N)nc23)CC1] | 1H NMR (300 MHz, DMSO-d6) δ = 8.26 (d, J = 8.9 Hz, 1H), 8.13 (d, J = 8.9 Hz, 1H), 7.21 (t, J = 7.7 Hz, 1H), 7.05 (d, J = 11.2 Hz, 1H), 6.80 (t, J = 8.1 Hz, 1H), 4.36-4.20 (m, 1H), 3.53 (s, 3H), 3.22 (s, 3H), 2.86 (d, J = 6.3 Hz, 2H), 2.24 (s, 3H), 2.08- 1.93 (m, 2H), 1.93-1.76 (m, 4H), 1.65-1.36 (m, 2H), 0.70-0.55 (m, 1H), 0.27 (d, J = 7.8 Hz, 2H), −0.01 (d, J = 4.5 Hz, 2H) | 500.4 |
| 118 | 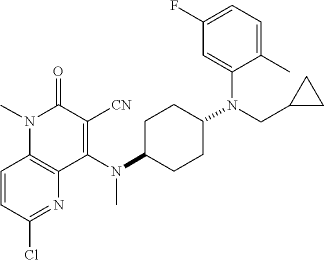 [SMILES: Cc1ccc(F)cc1N(CC1CC1)[C@H]1CC[C@H](N(C)c2c(C#N)c(=O)n(C)c3ccc(Cl)nc23)CC1] | 1H NMR (300 MHz, DMSO-d6) δ = 8.05 (d, J = 9.0 Hz, 1H), 7.79 (d, J = 8.9 Hz, 1H), 7.21 (t, J = 7.5 Hz, 1H), 7.05 (d, J = 11.3 Hz, 1H), 6.79 (t, J = 7.9 Hz, 1H), 4.32-4.14 (m, 1H), 3.52 (s, 3H), 3.18 (s, 3H), 2.85 (d, J = 6.1 Hz, 2H), 2.24 (s, 3H), 2.10- 1.93 (m, 2H), 1.93-1.72 (m, 4H), 1.62-1.32 (m, 2H), 0.71-0.55 (m, 1H), 0.26 (d, J = 7.5 Hz, 2H), 0.03-−0.05 (m, 2H) | 509.0 |
| 119 | 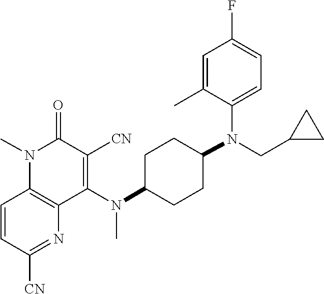 [SMILES: Cc1cc(F)ccc1N(CC1CC1)[C@H]1CC[C@@H](N(C)c2c(C#N)c(=O)n(C)c3ccc(C#N)nc23)CC1] | 1H NMR (300 MHz, DMSO-d6) δ = 8.25 (d, J = 8.8 Hz, 1H), 8.13 (d, J = 8.6 Hz, 1H), 7.33-7.23 (m, 1H), 7.11-7.03 (m, 1H), 6.98 (t, J = 8.3 Hz, 1H), 4.43-4.31 (m, 1H), 3.53 (s, 4H), 3.27 (s, 3H), 2.82- 2.69 (m, 2H), 2.48-2.39 (m, 3H), 2.16-1.78 (m, 4H), 1.77-1.56 (m, 2H), 1.56-1.37 (m, 2H), 0.80- 0.70 (m, 1H), 0.31 (d, J = 7.6 Hz, 2H), −0.09-−0.21 (m, 2H) | 498.9 |
| 120 | 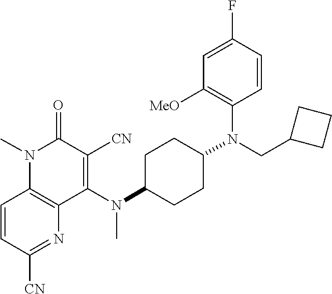 [SMILES: COc1cc(F)ccc1N(CC1CCC1)[C@H]1CC[C@H](N(C)c2c(C#N)c(=O)n(C)c3ccc(C#N)nc23)CC1] | (300 MHz, CDCl₃) δ ppm 7.85 (d, J = 8.6 Hz, 1H), 7.72 (d, J = 8.7 Hz, 1H), 6.97 (t, J = 7.5 Hz, 1H), 6.84-6.68 (m, 1H), 6.65-6.52 (m, 1H), 4.41-4.25 (m, 1H), 3.83 (s, 3H), 3.65 (s, 3H), 3.36 (s, 3H), 3.05 (bd, J = 7.1 Hz, 2H), 2.95 (bt, J = 11.6 Hz, 1H), 2.55-2.40 (m, 1 H), 2.37-2.17 (m, 2H), 2.17- 1.97 (m, 3H), 1.91-1.67 (m, 5H), 1.56-1.51 (m, 1H), 1.51-1.32 (m, 3H). | 529.50 |
| 121 | 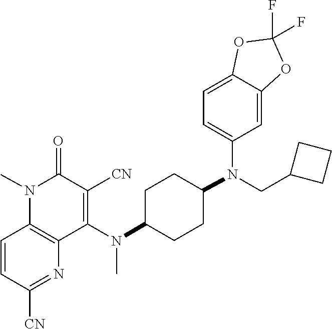 [SMILES: Cn1c(=O)c(C#N)c(N(C)[C@H]2CC[C@@H](N(CC3CCC3)c3ccc4c(c3)OC(F)(F)O4)CC2)c2nc(C#N)ccc21] | (400 MHz, DMSO-d6) δ ppm: 8.25 (d, J = 8.8 Hz, 1H), 8.14 (d, J = 9.0 Hz, 1H), 7.33-7.23 (m, 2H), 6.91 (d, J = 7.6 Hz, 1H), 4.43-4.32 (m, 1H), 3.54 (s, 3H), 2.99 (d, J = 7.1 Hz, 2H), 2.24-2.07 (m, 3H), 1.89-1.78 (m, 4H), 1.75-1.64 (m, 4H), 1.61- 1.52 (m, 2H), 1.50-1.39 (m, 2H). *Assumed 4H (1- Me, 1-CH) hidden behind residual water signal | 561.24 |
| 122 | 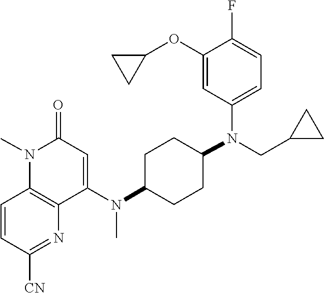 [SMILES: Cn1c(=O)cc(N(C)[C@H]2CC[C@@H](N(CC3CC3)c3ccc(F)c(OC4CC4)c3)CC2)c2nc(C#N)ccc21] | (400 MHz, DMSO) δ 8.12 (d, J = 8.8 Hz, 1H), 8.02 (d, J = 8.9 Hz, 1H), 7.18 (dd, J = 7.9, 2.4 Hz, 1H), 7.08 (dd, J = 11.3, 8.7 Hz, 1H), 6.77-6.70 (m, 1H), 5.91 (s, 1H), 4.35-4.26 (m, 1H), 3.93 (m, 1H), 3.50 (s, 3H), 3.41-3.37 (m, 1H), 2.90-2.87 (m, 5H), 2.08-1.96 (m, 2H), 1.91 (bd, J = 13.1 Hz, 2H), 1.57-1.49 (m, 2H), 1.44 (t, J = 12.8 Hz, 2H), 0.82- 0.63 (m, 5H), 0.37-0.29 (m, 2H), (−)0.03-(−)0.08 (m, 2H) | 516.27 |
| 123 | 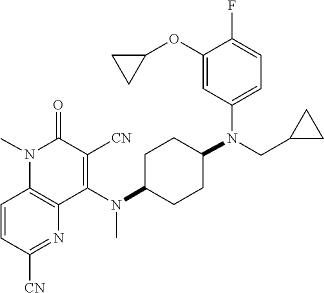 [SMILES: Cn1c(=O)c(C#N)c(N(C)[C@H]2CC[C@@H](N(CC3CC3)c3ccc(F)c(OC4CC4)c3)CC2)c2nc(C#N)ccc21] | (400 MHz, DMSO) δ 8.26 (d, J = 8.9 Hz, 1H), 8.15 (d, J = 8.9 Hz, 1H), 7.16 (dd, J = 7.7, 2.0 Hz, 1H), 7.08 (dd, J = 11.2, 8.7 Hz, 1H), 6.76-6.67 (m, 1H), 4.44-4.35 (m, 1H), 4.03-3.88 (m, 1H), 3.54 (s, 3H), 3.42 (br s, 1H), 3.29 (s, 3H), 2.91 (d, J = 6.3 Hz, 2H), 2.21-2.08 (m, 2H), 1.90-1.80 (m, 2H), 1.73- 1.64 (m, 2H), 1.48 (dd, J = 17.2, 6.5 Hz, 2H), 0.82- 0.65 (m, 5H), 0.38-0.31 (m, 2H), 0.03-(−)0.09 (m, 2H) | 541.28 |
| 124 | 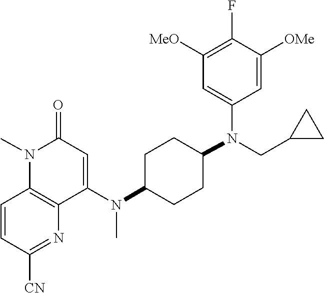 [SMILES: COc1cc(N(CC2CC2)[C@H]2CC[C@@H](N(C)c3cc(=O)n(C)c4ccc(C#N)nc34)CC2)cc(OC)c1F] | (400 MHz, DMSO-d6) δ ppm: 8.14 (d, J = 8.8 Hz, 1H), 8.04 (d, J = 8.9 Hz, 1H), 6.54 (d, J = 6.8 Hz, 2H), 5.94 (s, 1H), 4.35-4.26 (m, 1H), 3.80 (s, 6H), 3.51 (s, 3H), 3.46-3.41 (m, 1H), 2.91 (s, 3H), 2.90- 2.88 (m, 2H), 2.10-1.97 (m, 2H), 1.97-1.86 (m, 2H), 1.61-1.50 (m, 2H), 1.50-1.37 (m, 2H), 0.79- 0.67 (m, 1H), 0.39-0.30 (m, 2H), 0.01-(−)0.06 (m, 2H) | 520.29 |
| 125 | 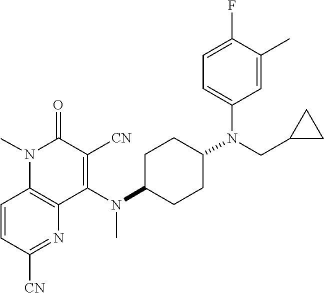 [SMILES: Cc1cc(N(CC2CC2)[C@H]2CC[C@H](N(C)c3c(C#N)c(=O)n(C)c4ccc(C#N)nc34)CC2)ccc1F] | (400 MHz, DMSO-d6) δ ppm: 8.26 (d, J = 8.9 Hz, 1H), 8.13 (d, J = 9.0 Hz, 1H), 6.94 (t, J = 9.2 Hz, 1H), 6.77-6.72 (m, 1H), 6.70-6.64 (m, 1H), 4.41- 4.31 (m, 1H), 3.62-3.55 (m, 1H), 3.53 (s, 3H), 3.28 (s, 3H), 2.98 (d, J = 5.8 Hz, 2H), 2.20 (d, J = 1.3 Hz, 3H), 2.07-1.98 (m, 4H), 1.89-1.77 (m, 2H), 1.63-1.48 (m, 2H), 0.92-0.83 (m, 1H), 0.47-0.41 (m, 2H), 0.22-0.17 (m, 2H) | 499.27 |
| 126 | 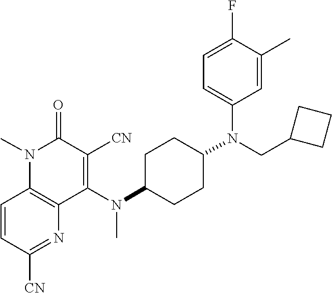 [SMILES: Cc1cc(N(CC2CCC2)[C@H]2CC[C@H](N(C)c3c(C#N)c(=O)n(C)c4ccc(C#N)nc34)CC2)ccc1F] | (400 MHz, DMSO-d6) δ ppm: 8.26 (d, J = 8.9 Hz, 1H), 8.13 (d, J = 8.9 Hz, 1H), 6.91 (t, J = 9.2 Hz, 1H), 6.73-6.63 (m, 1H), 6.63-6.53 (m, 1H), 4.43- 4.28 (m, 1H), 3.53 (s, 3H), 3.51-3.44 (m, 1H), 3.27 (s, 3H), 3.06 (d, J = 6.4 Hz, 2H), 2.43-2.30 (m, 1H), 2.18 (s, 3H), 2.07-1.97 (m, 4H), 1.96-1.87 (m, 2H), 1.83-1.50 (m, 8H) | 513.27 |
| 127 | 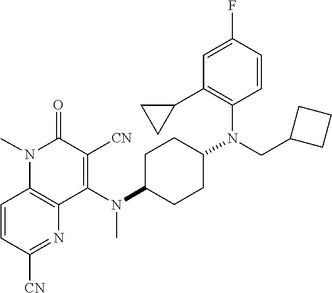 [SMILES: Cn1c(=O)c(C#N)c(N(C)[C@H]2CC[C@H](N(CC3CCC3)c3ccc(F)cc3C3CC3)CC2)c2nc(C#N)ccc21] | (400 MHz, DMSO) δ 8.26 (d, J = 8.9 Hz, 1H), 8.13 (d, J = 8.9 Hz, 1H), 7.19 (dd, J = 8.8, 5.7 Hz, 1H), 6.87 (td, J = 8.5, 3.0 Hz, 1H), 6.54 (dd, J = 10.6, 3.0 Hz, 1H), 4.35-4.19 (m, 1H), 3.54 (s, 3H), 3.22 (s, 3H), 3.03 (d, J = 7.4 Hz, 2H), 2.85-2.74 (m, 1H), 2.47-2.40 (m, 1H), 2.14-2.05 (m, 1H), 2.04- 1.96 (m, 2H), 1.97-1.87 (m, 2H), 1.85-1.68 (m, 6H), 1.62-1.42 (m, 4H), 0.99-0.90 (m, 2H), 0.70- 0.61 (m, 2H) | 539.29 |
| 128 | 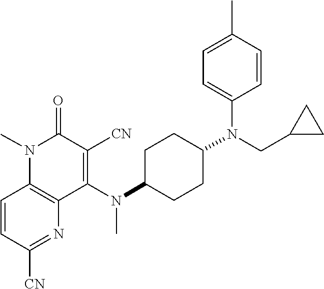 [SMILES: Cc1ccc(N(CC2CC2)[C@H]2CC[C@H](N(C)c3c(C#N)c(=O)n(C)c4ccc(C#N)nc34)CC2)cc1] | (400 MHz, DMSO) δ 8.26 (d, J = 8.9 Hz, 1H), 8.13 (d, J = 8.9 Hz, 1H), 6.99 (d, J = 8.4 Hz, 2H), 6.77 (d, J = 8.5 Hz, 2H), 4.43-4.31 (m, 1H), 3.68-3.57 (m, 1H), 3.53 (s, 3H), 3.28 (s, 3H), 2.99 (d, J = 5.7 Hz, 2H), 2.18 (s, 3H), 2.09-1.97 (m, 4H), 1.83 (bd, J = 11.7 Hz, 2H), 1.64-1.50 (m, 2H), 0.95-0.83 (m, 1H), 0.49-0.39 (m, 2H), 0.24-0.16 (m, 2H) | 481.27 |
| 129 | 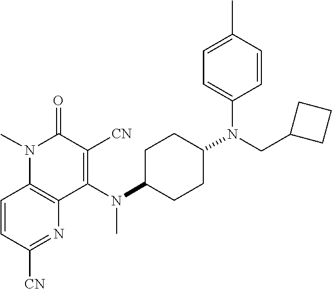 [SMILES: Cc1ccc(N(CC2CCC2)[C@H]2CC[C@H](N(C)c3c(C#N)c(=O)n(C)c4ccc(C#N)nc34)CC2)cc1] | (400 MHz, DMSO) δ 8.26 (d, J = 8.9 Hz, 1H), 8.13 (d, J = 8.9 Hz, 1H), 6.97 (d, J = 8.4 Hz, 2H), 6.68 (d, J = 8.6 Hz, 2H), 4.42-4.31 (m, 1H), 3.60-3.48 (m, 4H), 3.27 (s, 3H), 3.07 (d, J = 6.5 Hz, 2H), 2.45-2.34 (m, 1H), 2.17 (s, 3H), 2.07-1.97 (m, 4H), 1.97-1.87 (m, 2H), 1.83-1.50 (m, 8H) | 495.29 |
| 130 | 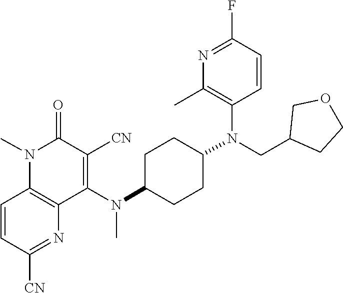 [SMILES: Cc1nc(F)ccc1N(CC1CCOC1)[C@H]1CC[C@H](N(C)c2c(C#N)c(=O)n(C)c3ccc(C#N)nc23)CC1] | — | 530.94 |
| 131 | 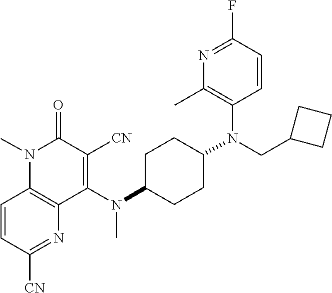 [SMILES: Cc1nc(F)ccc1N(CC1CCC1)[C@H]1CC[C@H](N(C)c2c(C#N)c(=O)n(C)c3ccc(C#N)nc23)CC1] | — | 514.36 |
| 132 | 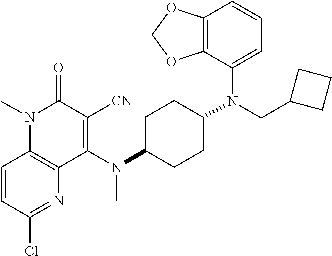 [SMILES: Cn1c(=O)c(C#N)c(N(C)[C@H]2CC[C@H](N(CC3CCC3)c3cccc4c3OCO4)CC2)c2nc(Cl)ccc21] | — | 534.42 |
| 133 | 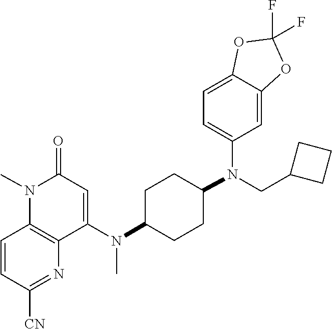 [SMILES: Cn1c(=O)cc(N(C)[C@H]2CC[C@@H](N(CC3CCC3)c3ccc4c(c3)OC(F)(F)O4)CC2)c2nc(C#N)ccc21] | (400 MHz, DMSO-d6) δ ppm: 8.13 (d, J = 8.8 Hz, 1H), 8.02 (d, J = 8.9 Hz, 1H), 7.29-7.25 (m, 2H), 6.93 (dd, J = 8.6, 2.0 Hz, 1H), 5.91 (s, 1H), 4.36- 4.23 (m, 1H), 3.50 (s, 3H), 3.23 (br s, 1H), 2.97 (d, J = 7.1 Hz, 2H), 2.89 (s, 3H), 2.23-2.08 (m, 1H), 2.08-1.93 (m, 2H), 1.90-1.77 (m, 4H), 1.78-1.65 (m, 2H), 1.61-1.47 (m, 4H), 1.40 (bt, J = 12.9 Hz, 2H) | 536.26 |
| 134 | 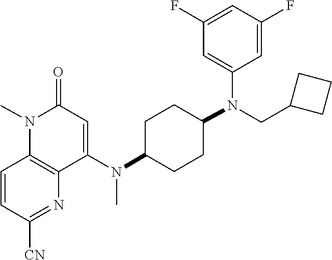 [SMILES: Cn1c(=O)cc(N(C)[C@H]2CC[C@@H](N(CC3CCC3)c3cc(F)cc(F)c3)CC2)c2nc(C#N)ccc21] | (400 MHz, DMSO-d6) δ ppm: 8.17 (d, J = 8.8 Hz, 1H), 8.08 (d, J = 8.9 Hz, 1H), 6.50-6.36 (m, 3H), 6.14 (s, 1H), 4.07-3.96 (m, 1H), 3.71-3.60 (m, 1H), 3.54 (s, 3H), 3.22 (d, J = 6.2 Hz, 2H), 2.92 (s, 3H), 2.44-2.30 (m, 1H), 2.14-2.01 (m, 2H), 1.95-1.85 (m, 2H), 1.83-1.59 (m, 8H), 1.53-1.41 (m, 2H) | 492.25 |
| 135 | 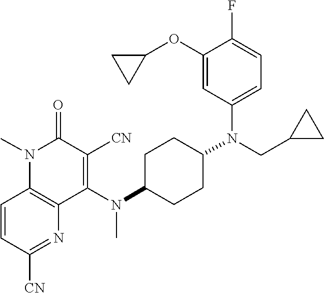 [SMILES: Cn1c(=O)c(C#N)c(N(C)[C@H]2CC[C@H](N(CC3CC3)c3ccc(F)c(OC4CC4)c3)CC2)c2nc(C#N)ccc21] | (400 MHz, DMSO) δ 8.26 (d, J = 8.9 Hz, 1H), 8.14 (d, J = 9.0 Hz, 1H), 6.99 (dd, J = 11.2, 9.0 Hz, 1H), 6.85 (dd, J = 7.4, 2.9 Hz, 1H), 6.39 (dt, J = 8.9, 3.1 Hz, 1H), 4.43-4.32 (m, 1H), 3.93 (tt, J = 6.1, 2.9 Hz, 1H), 3.60 (tt, J = 11.7, 3.0 Hz, 1H), 3.54 (s, 3H), 3.29 (s, 3H), 3.03 (d, J = 5.7 Hz, 2H), 2.09- 1.97 (m, 4H), 1.87 (bd, J = 11.5 Hz, 2H), 1.66-1.53 (m, 2H), 0.92 (ddd, J = 13.3, 8.1, 5.3 Hz, 1H), 0.82-0.75 (m, 2H), 0.74-0.68 (m, 2H), 0.52-0.44 (m, 2H), 0.27-0.20 (m, 2H) | 541.29 |
| 136 | 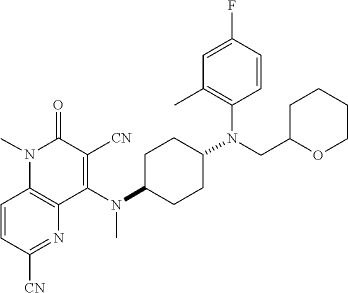 [SMILES: Cc1cc(F)ccc1N(CC1CCCCO1)[C@H]1CC[C@H](N(C)c2c(C#N)c(=O)n(C)c3ccc(C#N)nc23)CC1] | (400 MHz, DMSO) δ 8.26 (d, J = 8.9 Hz, 1H), 8.13 (d, J = 8.9 Hz, 1H), 7.24 (dd, J = 8.8, 5.5 Hz, 1H), 7.04 (dd, J = 9.8, 3.1 Hz, 1H), 6.95 (td, J = 8.5, 3.1 Hz, 1H), 4.37-4.23 (m, 1H), 3.85-3.72 (m, 1H), 3.54 (s, 3H), 3.21 (s, 3H), 3.19-3.10 (m, 2H), 2.97- 2.90 (m, 1H), 2.86-2.77 (m, 1H), 2.71-2.60 (m, 1H), 2.26 (s, 3H), 2.06-1.93 (m, 2H), 1.93-1.78 (m, 4H), 1.74-1.65 (m, 1H), 1.64-1.56 (m, 1H), 1.48- 1.42 (m, 1H), 1.41-1.19 (m, 4H), 1.16-0.97 (m, 1H) | 543.30 |
| 137 | 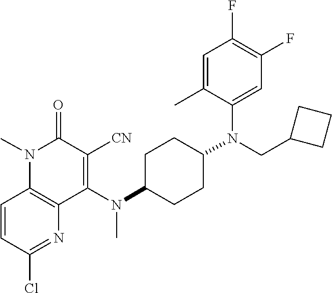 [SMILES: Cc1cc(F)c(F)cc1N(CC1CCC1)[C@H]1CC[C@H](N(C)c2c(C#N)c(=O)n(C)c3ccc(Cl)nc23)CC1] | 1H NMR (300 MHz, DMSO-d6) δ = 8.06 (d, J = 8.9 Hz, 1H), 7.81 (d, J = 8.7 Hz, 1H), 7.29-7.19 (m, 2H), 4.32-4.18 (m, 1H), 3.52 (s, 3H), 3.18 (s, 3H), 2.97 (d, J = 7.5 Hz, 2H), 2.70-2.54 (m, 1H), 2.19 (s, 3H), 2.06-1.95 (m, 3H), 1.91-1.67 (m, 8H), 1.62-1.44 (m, 4H), 1.24 (s, 1H) | 541.0 |
| 138 | 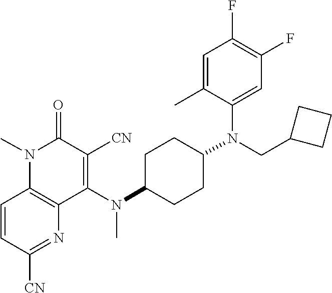 [SMILES: Cc1cc(F)c(F)cc1N(CC1CCC1)[C@H]1CC[C@H](N(C)c2c(C#N)c(=O)n(C)c3ccc(C#N)nc23)CC1] | 1H NMR (300 MHz, DMSO-d6) δ = 8.27 (d, J = 8.5 Hz, 1H), 8.14 (d, J = 8.9 Hz, 1H), 7.29-7.19 (m, 2H), 4.30 (s, 1H), 3.54 (s, 3H), 3.22 (s, 3H), 2.97 (d, J = 7.1 Hz, 2H), 2.70-2.54 (m, 1H), 2.19 (s, 3H), 2.10-1.93 (m, 4H), 1.92-1.67 (m, 8H), 1.65- 1.44 (m, 4H), 0.93-0.81 (m, 1H) | 532.3 |
| 139 | 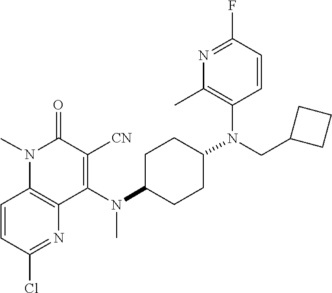 [SMILES: Cc1nc(F)ccc1N(CC1CCC1)[C@H]1CC[C@H](N(C)c2c(C#N)c(=O)n(C)c3ccc(Cl)nc23)CC1] | (300 MHz, CHLOROFORM-d) δ ppm 7.68-7.62 (m, 1H) 7.60-7.49 (m, 2H) 6.78 (br s, 1H) 4.94- 4.43 (m, 2 H) 4.36 (bt, J = 10.6 Hz, 1H) 3.64 (s, 3 H) 3.31 (s, 3 H) 3.22-3.02 (m, 1 H) 2.57 (br s, 3H) 2.45-2.16 (br s, 3 H) 2.14-1.95 (m, 2 H) 1.95-1.72 (m, 6H) 1.59 (br s, 4H) | — |
| 140 | 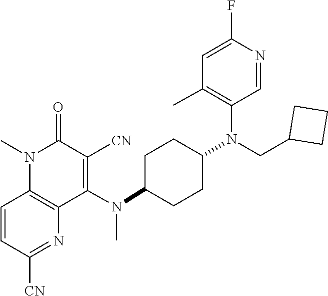 [SMILES: Cc1cc(F)ncc1N(CC1CCC1)[C@H]1CC[C@H](N(C)c2c(C#N)c(=O)n(C)c3ccc(C#N)nc23)CC1] | (300 MHz, CDCl₃) δ ppm 8.07 (br s, 1H) 7.87 (d, J = 8.3 Hz, 1H) 7.74 (d, J = 8.8 Hz, 1H), 6.85 (br s, 1H) 4.69 (br s, 2H) 4.40-4.26 (m, 1H) 3.66 (s, 3H) 3.46-3.21 (m, 4H) 2.63-2.45 (m, 3H) 2.28-2.10 (m, 4H), 1.95-1.43 (m, 11H) | — |
| 14 | 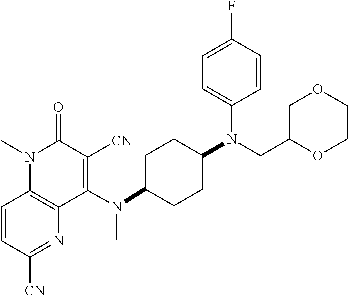 [SMILES: Cn1c(=O)c(C#N)c(N(C)[C@H]2CC[C@@H](N(CC3COCCO3)c3ccc(F)cc3)CC2)c2nc(C#N)ccc21] | (400 MHz, DMSO-d6) δ ppm: 8.25 (d, J = 8.9 Hz, 1H), 8.14 (d, J = 9.0 Hz, 1H), 7.24 (dd, J = 9.0, 5.0 Hz, 2H), 7.19-7.08 (m, 2H), 4.46-4.27 (m, 1H), 3.79 (dd, J = 11.1, 1.9 Hz, 1H), 3.64 (d, J = 8.3 Hz, 1H), 3.58 (d, J = 8.2 Hz, 1H), 3.54 (s, 3H), 3.47- 3.39 (m, 2H), 3.29 (s, 3H), 3.23-3.15 (m, 1H), 3.01 (d, J = 6.1 Hz, 2H), 2.18-2.03 (m, 2H), 1.99-1.91 (m, 1H), 1.76-1.56 (m, 3H), 1.55-1.35 (m, 2H) | 531.26 |
| 142 | 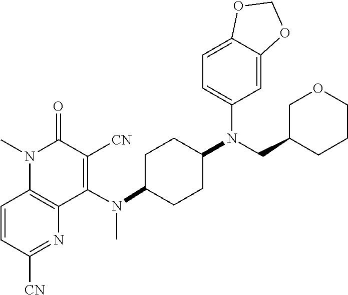 [SMILES: Cn1c(=O)c(C#N)c(N(C)[C@H]2CC[C@@H](N(C[C@@H]3CCCOC3)c3ccc4c(c3)OCO4)CC2)c2nc(C#N)ccc21] | (400 MHz, Chloroform-d) δ 7.83 (d, J = 8.7 Hz, 1H), 7.70 (d, J = 8.8 Hz, 1H), 6.79-6.73 (m, 2H), 6.72-6.66 (m, 1H), 5.97 (s, 2H), 4.53-4.42 (m, 1H), 4.02 (dd, J = 10.8, 3.7 Hz, 1H), 3.86-3.78 (m, 1H), 3.63 (s, 3H), 3.43 (s, 3H), 3.42-3.36 (m, 4H), 3.21- 3.13 (m, 1H), 2.97 (br s, 1H), 2.74 (dd, J = 12.4, 8.1 Hz, 1H), 2.65 (dd, J = 12.4, 6.0 Hz, 1H), 2.30- 2.16 (m, 2H), 2.09-2.00 (m, 1H), 1.93-1.78 (m, 4H), 1.65-1.39 (m, 5H), 1.27-1.14 (m, 1H) | 555.20 |
| 143 | 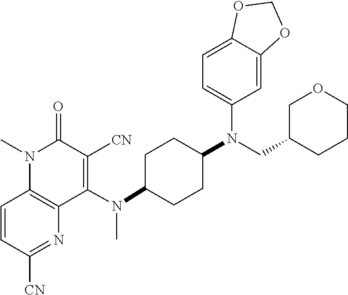 [SMILES: Cn1c(=O)c(C#N)c(N(C)[C@H]2CC[C@@H](N(C[C@H]3CCCOC3)c3ccc4c(c3)OCO4)CC2)c2nc(C#N)ccc21] | (400 MHz, Chloroform-d) 8 7.83 (d, J = 8.7 Hz, 1H), 7.70 (d, J = 8.8 Hz, 1H), 6.79-6.73 (m, 2H), 6.72-6.66 (m, 1H), 5.97 (s, 2H), 4.53-4.42 (m, 1H), 4.02 (dd, J = 10.8, 3.7 Hz, 1H), 3.86-3.78 (m, 1H), 3.63 (s, 3H), 3.43 (s, 3H), 3.42-3.36 (m, 4H), 3.21- 3.13 (m, 1H), 2.97 (br s, 1H), 2.74 (dd, J = 12.4, 8.1 Hz, 1H), 2.65 (dd, J = 12.4, 6.0 Hz, 1H), 2.30- 2.16 (m, 2H), 2.09-2.00 (m, 1H), 1.93-1.78 (m, 4H), 1.65-1.39 (m, 5H), 1.27-1.14 (m, 1H) | 555.20 |
| 144 | 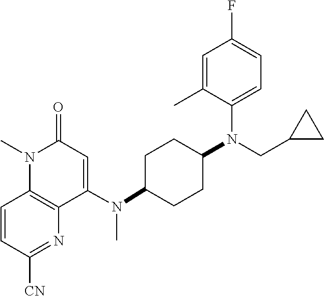 [SMILES: Cc1cc(F)ccc1N(CC1CC1)[C@H]1CC[C@@H](N(C)c2cc(=O)n(C)c3ccc(C#N)nc23)CC1] | 1H NMR (300 MHz, DMSO-d6) δ = 8.14 (d, J = 8.8 Hz, 1H), 8.03 (d, J = 8.8 Hz, 1H), 7.34-7.23 (m, 1H), 7.06 (d, J = 9.8 Hz, 1H), 6.97 (t, J = 8.6 Hz, 1H), 5.89 (s, 1H), 4.42-4.30 (m, 1H), 3.56-3.47 (m, 4H), 2.86 (s, 3H), 2.80-2.54 (m, 2H), 2.43 (s, 3H), 2.07-1.70 (m, 4H), 1.63-1.33 (m, 4H), 0.86-0.68 (m, 1H), 0.30 (d, J = 7.7 Hz, 2H), −0.08-−0.25 (m, 2H) | 475.0 |
| 145 | 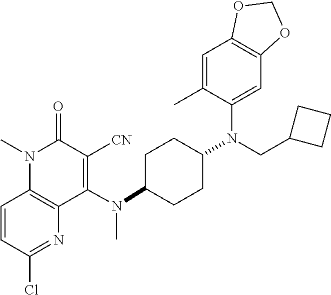 [SMILES: Cc1cc2c(cc1N(CC1CCC1)[C@H]1CC[C@H](N(C)c3c(C#N)c(=O)n(C)c4ccc(Cl)nc34)CC1)OCO2] | 1H NMR (300 MHz, DMSO-d6) δ = 8.05 (d, J = 8.9 Hz, 1H), 7.80 (d, J = 8.6 Hz, 1H), 6.82 (s, 1H), 6.74 (s, 1H), 5.93 (s, 2H), 4.31-4.16 (m, 1H), 3.52 (s, 3H), 3.18 (s, 2H), 2.92 (d, J = 4.7 Hz, 3H), 2.13 (s, 3H), 2.00 (d, J = 9.2 Hz, 3H), 1.93-1.64 (m, 8H), 1.63-1.34 (m, 5H). LCMS (ES, m/z) | 548.0 |
| 146 | 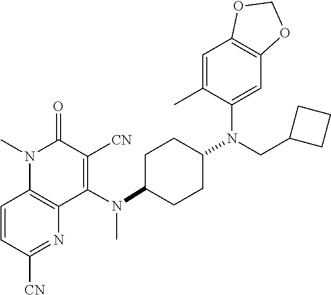 [SMILES: Cc1cc2c(cc1N(CC1CCC1)[C@H]1CC[C@H](N(C)c3c(C#N)c(=O)n(C)c4ccc(C#N)nc34)CC1)OCO2] | 1H NMR (300 MHz, DMSO-d6) δ = 8.25 (d, J = 8.8 Hz, 1H), 8.13 (d, J = 8.9 Hz, 1H), 6.81 (s, 1H), 6.74 (s, 1H), 5.92 (s, 2H), 4.29 (t, J = 10.8 Hz, 1H), 3.53 (s, 3H), 3.21 (s, 3H), 2.96-2.88 (m, J = 6.9 Hz, 2H), 2.66-2.53 (m, 1H), 2.13 (s, 3H), 2.03-1.66 (m, 10H), 1.62-1.32 (m, 5H) | 539.5 |
| 147 | 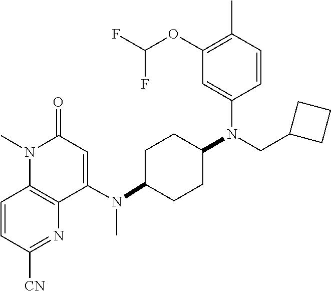 [SMILES: Cc1ccc(N(CC2CCC2)[C@H]2CC[C@@H](N(C)c3cc(=O)n(C)c4ccc(C#N)nc34)CC2)cc1OC(F)F] | (400 MHz, DMSO) δ 8.14 (d, J = 8.8 Hz, 1H), 8.04 (d, J = 8.9 Hz, 1H), 7.34-6.92 (m, 2H), 6.88 (dd, J = 8.0, 1.9 Hz, 1H), 6.81 (s, 1H), 5.96 (s, 1H), 4.32- 4.17 (m, 1H), 3.52 (s, 3H), 3.36-3.32 (m, 1H), 3.03 (d, J = 6.9 Hz, 2H), 2.89 (s, 3H), 2.23 (dt, J = 15.4, 7.7 Hz, 1H), 2.15 (s, 3H), 2.08-1.95 (m, 2H), 1.91- 1.78 (m, 4H), 1.77-1.64 (m, 2H), 1.63-1.49 (m, 4H), 1.48-1.35 (m, 2H) | 536.29 |
| 148 | 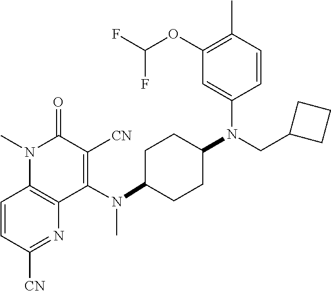 [SMILES: Cc1ccc(N(CC2CCC2)[C@H]2CC[C@@H](N(C)c3c(C#N)c(=O)n(C)c4ccc(C#N)nc34)CC2)cc1OC(F)F] | (400 MHz, DMSO) δ 8.26 (d, J = 8.9 Hz, 1H), 8.16 (d, J = 8.9 Hz, 1H), 7.34-6.91 (m, 2H), 6.83 (d, J = 8.2 Hz, 1H), 6.76 (s, 1H), 4.42-4.32 (m, 1H), 3.55 (s, 3H), 3.37 (s, 1H), 3.27 (s, 3H), 3.03 (d, J = 6.8 Hz, 2H), 2.31-2.19 (m, 1H), 2.19-2.05 (m, 5H), 1.89-1.74 (m, 4H), 1.74-1.62 (m, 4H), 1.62-1.50 (m, 2H), 1.50-1.38 (m, 2H) | 561.29 |
| 149 | 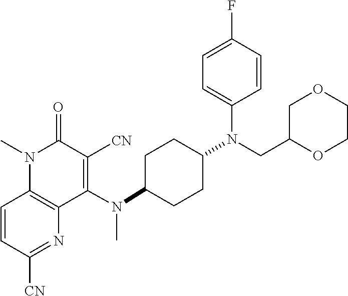 [SMILES: Cn1c(=O)c(C#N)c(N(C)[C@H]2CC[C@H](N(CC3COCCO3)c3ccc(F)cc3)CC2)c2nc(C#N)ccc21] | (400 MHz, DMSO) δ 8.27 (d, J = 8.9 Hz, 1H), 8.15 (d, J = 9.0 Hz, 1H), 7.07-6.99 (m, 2H), 6.90 (dd, J = 9.2, 4.5 Hz, 2H), 4.46-4.24 (m, 1H), 3.78-3.69 (m, 2H), 3.64-3.57 (m, 1H), 3.58-3.50 (m, 4H), 3.51-3.45 (m, 2H), 3.43-3.35 (m, 1H), 3.28 (s, 3H), 3.25-3.18 (m, 1H), 3.10 (d, J = 5.7 Hz, 2H), 2.10-1.94 (m, 4H), 1.91-1.72 (m, 2H), 1.68-1.47 (m, 2H) | 531.25 |
| 150 | 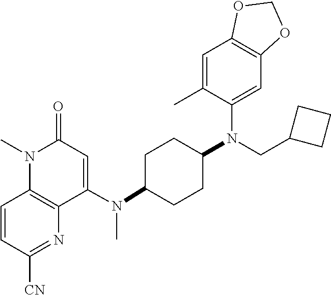 [SMILES: Cc1cc2c(cc1N(CC1CCC1)[C@H]1CC[C@@H](N(C)c3cc(=O)n(C)c4ccc(C#N)nc34)CC1)OCO2] | 1H NMR (300 MHz, DMSO-d6) δ = 8.11 (d, J = 8.7 Hz, 1H), 8.01 (d, J = 8.8 Hz, 1H), 6.80 (s, 1H), 6.74 (s, 1H), 5.93 (s, 2H), 5.85 (s, 1H), 4.31 (t, J = 10.4 Hz, 1H), 3.49 (s, 2H), 3.19 (s, 1H), 2.84 (s, 3H), 2.38-2.21 (m, 4H), 1.97-1.62 (m, 5H), 1.61- 1.34 (m, J = 8.6 Hz, 4H), 1.18 (s, 2H) | 514.5 |
| 151 | 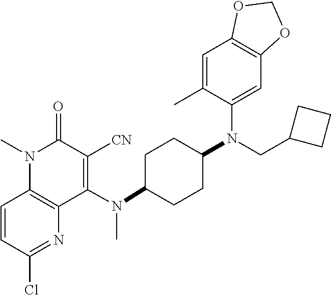 [SMILES: Cc1cc2c(cc1N(CC1CCC1)[C@H]1CC[C@@H](N(C)c3c(C#N)c(=O)n(C)c4ccc(Cl)nc34)CC1)OCO2] | 1H NMR (300 MHz, DMSO-d6) δ = 8.05 (d, J = 9.2 Hz, 1H), 7.79 (d, J = 9.0 Hz, 1H), 6.82 (s, 1H), 6.76 (s, 1H), 5.94 (s, 2H), 4.39-4.25 (m, 1H), 3.52 (s, 3H), 3.28-3.15 (m, 4H), 2.94-2.78 (m, 2H), 2.36-2.24 (m, 4H), 1.96-1.64 (m, 5H), 1.60-1.44 (m, 3H) | 548.1 |
| 152 | 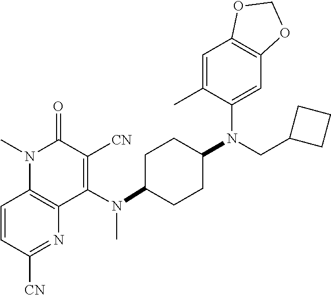 [SMILES: Cc1cc2c(cc1N(CC1CCC1)[C@H]1CC[C@@H](N(C)c3c(C#N)c(=O)n(C)c4ccc(C#N)nc34)CC1)OCO2] | 1H NMR (300 MHz, DMSO-d6) δ = 8.25 (d, J = 8.5 Hz, 1H), 8.13 (d, J = 9.0 Hz, 1H), 6.83 (s, 1H), 6.76 (s, 1H), 5.94 (s, 2H), 4.41-4.28 (m, 1H), 3.53 (s, 3H), 3.31-3.18 (m, 4H), 3.04-2.70 (m, 2H), 2.37-2.18 (m, 5H), 1.91-1.66 (m, 6H), 1.61-1.41 (m, 4H) | 539.0 |
| 153 | 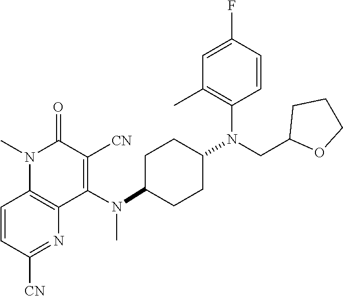 [SMILES: Cc1cc(F)ccc1N(CC1CCCO1)[C@H]1CC[C@H](N(C)c2c(C#N)c(=O)n(C)c3ccc(C#N)nc23)CC1] | (400 MHz, DMSO) δ 8.25 (d, J = 8.9 Hz, 1H), 8.13 (d, J = 8.9 Hz, 1H), 7.25 (dd, J = 8.8, 5.6 Hz, 1H), 7.03 (dd, J = 9.8, 3.0 Hz, 1H), 6.94 (td, J = 8.5, 3.1 Hz, 1H), 4.35-4.25 (m, 1H), 3.70-3.62 (m, 1H), 3.53 (s, 3H), 3.51-3.45 (m, 2H), 3.20 (s, 3H), 3.19- 3.15 (m, 1H), 2.87-2.75 (m, 1H), 2.70-2.58 (m, 1H), 2.26 (s, 3H), 2.03-1.93 (m, 2H), 1.91-1.81 (m, 3H), 1.79-1.62 (m, 4H), 1.60-1.40 (m, 3H) | 529.31 |
| 154 | 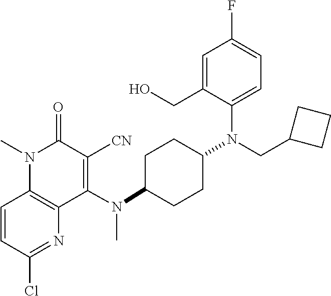 [SMILES: Cn1c(=O)c(C#N)c(N(C)[C@H]2CC[C@H](N(CC3CCC3)c3ccc(F)cc3CO)CC2)c2nc(Cl)ccc21] | 1H NMR (300 MHz, DMSO-d6) δ = 8.05 (d, J = 8.9 Hz, 1H), 7.80 (d, J = 8.8 Hz, 1H), 7.26-7.16 (m, 2H), 7.03-6.93 (m, 1H), 5.26-5.10 (m, 1H), 4.54 (s, 2H), 4.31-4.16 (m, 1H), 3.52 (s, 3H), 3.17 (s, 3H), 2.96 (d, J = 7.0 Hz, 2H), 2.62-2.54 (m, 1H), 2.08-1.92 (m, J = 8.7 Hz, 3H), 1.90-1.65 (m, 8H), 1.59-1.45 (m, 4H) | 538.5 |
| 155 | 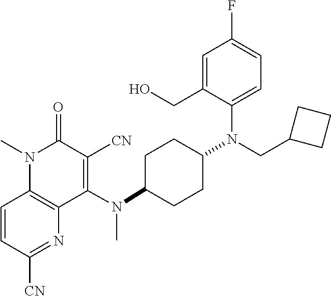 [SMILES: Cn1c(=O)c(C#N)c(N(C)[C@H]2CC[C@H](N(CC3CCC3)c3ccc(F)cc3CO)CC2)c2nc(C#N)ccc21] | 1H NMR (300 MHz, DMSO-d6) δ = 8.26 (d, J = 8.8 Hz, 1H), 8.14 (d, J = 8.9 Hz, 1H), 7.26-7.16 (m, 2H), 6.98 (t, J = 8.3 Hz, 1H), 5.18 (t, J = 5.2 Hz, 1H), 4.55 (d, J = 5.0 Hz, 2H), 4.29 (t, J = 10.4 Hz, 1H), 3.53 (s, 3H), 3.20 (s, 3H), 2.97 (d, J = 7.0 Hz, 2H), 2.64-2.53 (m, 1H), 2.10-1.91 (m, 3H), 1.90- 1.64 (m, 8H), 1.59-1.43 (m, 4H) | 530.2 |
| 156 | 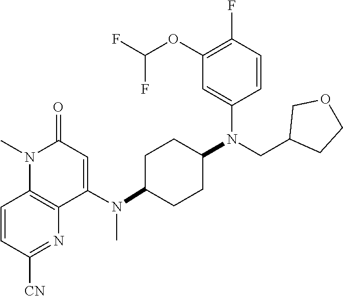 [SMILES: Cn1c(=O)cc(N(C)[C@H]2CC[C@@H](N(CC3CCOC3)c3ccc(F)c(OC(F)F)c3)CC2)c2nc(C#N)ccc21] | (400 MHz, DMSO-d6) δ ppm: 8.14 (d, J = 8.8 Hz, 1H), 8.04 (d, J = 8.9 Hz, 1H), 7.37-7.29 (m, J = 19.3 Hz, 1H), 7.26 (t, J = 73.5 Hz, 1H), 7.20-7.10 (m, 2H), 5.94 (s, 1H), 4.37-4.21 (m, 1H), 3.74-3.60 (m, 2H), 3.58-3.52 (m, 1H), 3.51 (s, 3H), 3.38-3.33 (m, 1H), 3.27-3.20 (m, 1H), 2.97 (d, J = 6.0 Hz, 2H), 2.89 (s, 3H), 2.08-1.74 (m, 6H), 1.60-1.36 (m, 5H) | 556.29 |
| 157 | 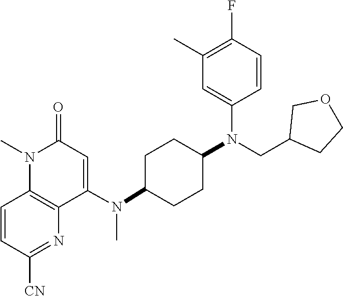 [SMILES: Cc1cc(N(CC2CCOC2)[C@H]2CC[C@@H](N(C)c3cc(=O)n(C)c4ccc(C#N)nc34)CC2)ccc1F] | (400 MHz, DMSO-d6) δ ppm: 8.13 (d, J = 8.8 Hz, 1H), 8.03 (d, J = 8.9 Hz, 1H), 7.17 (d, J = 7.3 Hz, 1H), 7.08 (d, J = 7.6 Hz, 2H), 5.91 (s, 1H), 4.39- 4.25 (m, 1H), 3.73-3.63 (m, 2H), 3.57-3.52 (m, 1H), 3.51 (s, 3H), 3.37 (dd, J = 8.0, 6.2 Hz, 1H), 3.12 (s, 1H), 2.90 (s, 3H), 2.89-2.87 (m, 2H), 2.22 (s, 3H), 2.07-1.70 (m, 6H), 1.57-1.32 (m, 5H) | 504.34 |
| 158 | 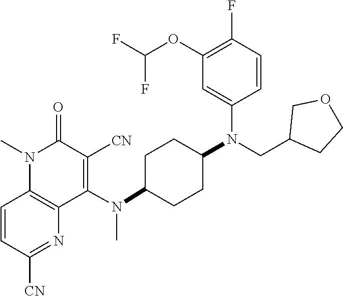 [SMILES: Cn1c(=O)c(C#N)c(N(C)[C@H]2CC[C@@H](N(CC3CCOC3)c3ccc(F)c(OC(F)F)c3)CC2)c2nc(C#N)ccc21] | (400 MHz, DMSO-d6) δ ppm: 8.25 (d, J = 8.9 Hz, 1H), 8.15 (d, J = 8.9 Hz, 1H), 7.36-7.27 (m, J = 9.0 Hz, 1H), 7.25 (t, J = 73.5 Hz, 1H), 7.17-7.08 (m, J = 3.9 Hz, 2H), 4.42-4.32 (m, 1H), 3.74-3.60 (m, 2H), 3.54 (s, 3H), 3.57-3.50 (m, 1H), 3.42-3.27 (m, 2H), 3.29 (s, 3H), 3.02-2.94 (m, 2H), 2.18-1.98 (m, 3H), 1.92-1.79 (m, 2H), 1.79-1.62 (m, 3H), 1.55- 1.40 (m, 3H) | 581.30 |
| 159 | 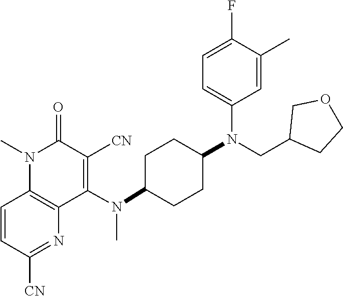 [SMILES: Cc1cc(N(CC2CCOC2)[C@H]2CC[C@@H](N(C)c3c(C#N)c(=O)n(C)c4ccc(C#N)nc34)CC2)ccc1F] | (400 MHz, DMSO-d6) δ ppm: 8.25 (d, J = 8.9 Hz, 1H), 8.14 (d, J = 8.9 Hz, 1H), 7.17 (d, J = 7.3 Hz, 1H), 7.07 (d, J = 8.0 Hz, 2H), 4.45-4.28 (m, 1H), 3.73-3.63 (m, 2H), 3.59-3.50 (m, 1H), 3.54 (s, 3H), 3.40-3.37 (m, 1H), 3.31 (s, 3H), 3.15 (s, 1H), 2.90 (d, J = 7.3 Hz, 2H), 2.24 (s, 3H), 2.21-2.08 (m, 2H), 2.04-1.80 (m, 3H), 1.79-1.59 (m, 3H), 1.57- 1.36 (m, 3H) | 529.33 |
| 160 | 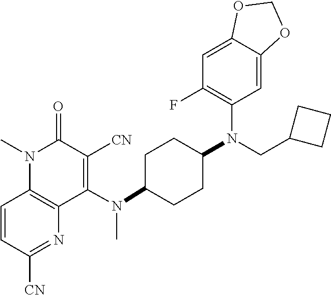 [SMILES: Cn1c(=O)c(C#N)c(N(C)[C@H]2CC[C@@H](N(CC3CCC3)c3cc4c(cc3F)OCO4)CC2)c2nc(C#N)ccc21] | (400 MHz, DMSO-d6) δ ppm: 8.24 (d, J = 8.9 Hz, 1H), 8.12 (d, J = 8.9 Hz, 1H), 6.95 (d, J = 6.7 Hz, 1H), 6.91 (d, J = 10.2 Hz, 1H), 6.03 (s, 2H), 4.42- 4.29 (m, 1H), 3.52 (s, 3H), 3.27 (s, 3H), 3.17 (br s, 1H), 2.88 (d, J = 7.2 Hz, 2H), 2.25-2.07 (m, 3H), 1.93-1.71 (m, 6H), 1.69-1.53 (m, 4H), 1.44 (t, J = 13.3 Hz, 2H) | 543.29 |
| 161 | 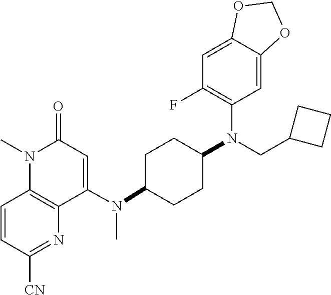 [SMILES: Cn1c(=O)cc(N(C)[C@H]2CC[C@@H](N(CC3CCC3)c3cc4c(cc3F)OCO4)CC2)c2nc(C#N)ccc21] | (400 MHz, DMSO) δ 8.12 (d, J = 8.8 Hz, 1H), 8.02 (d, J = 8.9 Hz, 1H), 6.91 (dd, J = 11.7,8.5 Hz, 2H), 6.03 (s, 2H), 5.87 (s, 1H), 4.41-4.29 (m, 1H), 3.50 (s, 3H), 3.16 (s, 1H), 2.93-2.79 (m, 5H), 2.23-2.13 (m, 1H), 2.09-1.94 (m, 2H), 1.92-1.67 (m, 6H), 1.63-1.45 (m, 4H), 1.44-1.31 (m, 2H) | 518.29 |
| 162 | 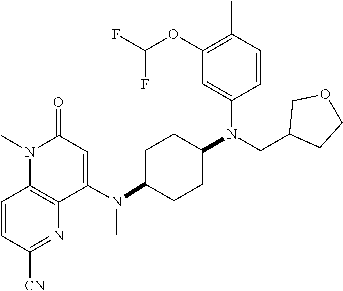 [SMILES: Cc1ccc(N(CC2CCOC2)[C@H]2CC[C@@H](N(C)c3cc(=O)n(C)c4ccc(C#N)nc34)CC2)cc1OC(F)F] | (400 MHz, DMSO) δ 8.14 (d, J = 8.8 Hz, 1H), 8.03 (d, J = 8.9 Hz, 1H), 7.38-6.97 (m, 3H), 6.94 (s, 1H), 5.94 (s, 1H), 4.34-4.23 (m, 1H), 3.72-3.60 (m, 2H), 3.57-3.51 (m, 1H), 3.51 (s, 3H), 3.42-3.33 (m, 1H), 3.26 (br s, 1H), 3.02-2.95 (m, 2H), 2.89 (s, 3H), 2.18 (s, 3H), 2.10-1.94 (m, 3H), 1.95-1.73 (m, 3H), 1.60-1.34 (m, 5H) | 552.33 |
| 163 | 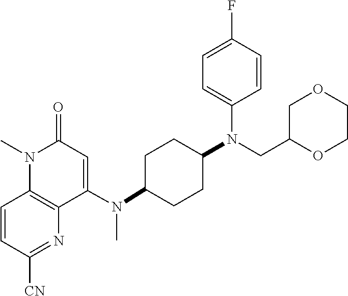 [SMILES: Cn1c(=O)cc(N(C)[C@H]2CC[C@@H](N(CC3COCCO3)c3ccc(F)cc3)CC2)c2nc(C#N)ccc21] | (400 MHz, DMSO-d6) δ 8.14 (d, J = 8.8 Hz, 1H), 8.04 (d, J = 8.9 Hz, 1H), 7.31-7.22 (m, 2H), 7.20- 7.09 (m, 2H), 5.92 (s, 1H), 4.36-4.26 (m, 1H), 3.79 (dd, J = 11.3, 2.3 Hz, 1H), 3.65 (d, J = 8.2 Hz, 1H), 3.58 (d, J = 8.1 Hz, 1H), 3.51 (s, 3H), 3.42 (d, J = 8.2 Hz, 2H), 3.32-3.26 (m, 2H), 3.23-3.17 (m, 1H), 3.01 (d, J = 6.1 Hz, 2H), 2.89 (s, 3H), 2.07-1.90 (m, 3H), 1.71-1.32 (m, 5H) | 506.20 |
| 164 | 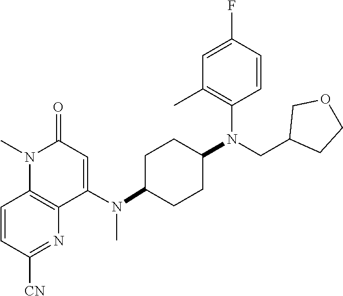 [SMILES: Cc1cc(F)ccc1N(CC1CCOC1)[C@H]1CC[C@@H](N(C)c2cc(=O)n(C)c3ccc(C#N)nc23)CC1] | (400 MHz, DMSO) δ 8.14 (d, J = 8.8 Hz, 1H), 8.03 (d, J = 8.9 Hz, 1H), 7.28 (dd, J = 8.8,5.7 Hz, 1H), 7.07 (dd, J = 9.9, 3.0 Hz, 1H), 6.99 (td, J = 8.4, 3.1 Hz, 1H), 5.90 (s, 1H), 4.41-4.25 (m, 1H), 3.69-3.57 (m, 2H), 3.56-3.46 (m, 4H), 3.18-3.09 (m, 1H), 3.00-2.88 (m, 2H), 2.87 (s, 3H), 2.41 (s, 3H), 2.17- 2.09 (m, 2H), 1.93-1.80 (m, 2H), 1.77-1.44 (m, 4H), 1.37 (m, 3H) | 504.32 |
| 165 | 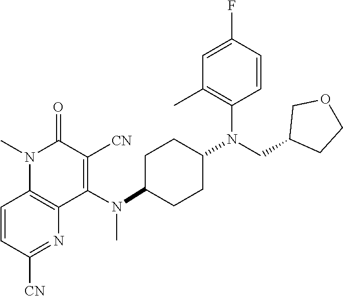 [SMILES: Cc1cc(F)ccc1N(C[C@H]1CCOC1)[C@H]1CC[C@H](N(C)c2c(C#N)c(=O)n(C)c3ccc(C#N)nc23)CC1] | (400 MHz, DMSO) δ 8.26 (d, J = 8.9 Hz, 1H), 8.14 (d, J = 8.9 Hz, 1H), 7.24 (dd, J = 8.8, 5.6 Hz, 1H), 7.06 (dd, J = 9.8, 2.9 Hz, 1H), 6.96 (td, J = 8.5, 3.0 Hz, 1H), 4.35-4.20 (m, 1H), 3.71-3.63 (m, 1H), 3.53 (s, 3H), 3.50-3.46 (m, 2H), 3.43-3.32 (m, 1H) 3.21 (s, 3H), 3.06-2.83 (m, 2H), 2.70-2.59 (m, 1H), 2.26 (s, 3H), 2.05-1.96 (m, 3H), 1.96-1.91 (m, 1H), 1.90-1.79 (m, 4H), 1.79-1.71 (m, 1H), 1.63-1.44 (m, 3H) | 529.33 |
| 166 | 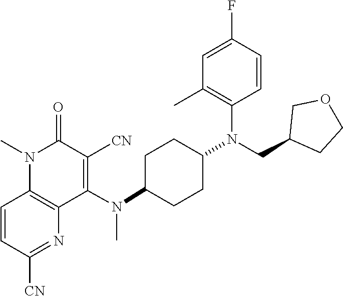 [SMILES: Cc1cc(F)ccc1N(C[C@@H]1CCOC1)[C@H]1CC[C@H](N(C)c2c(C#N)c(=O)n(C)c3ccc(C#N)nc23)CC1] | (400 MHz, DMSO) δ 8.26 (d, J = 8.9 Hz, 1H), 8.14 (d, J = 8.9 Hz, 1H), 7.24 (dd, J = 8.8, 5.6 Hz, 1H), 7.06 (dd, J = 9.8, 2.9 Hz, 1H), 6.96 (td, J = 8.5,3.0 Hz, 1H), 4.35-4.20 (m, 1H), 3.71-3.63 (m, 1H). 3.53 (s, 3H), 3.50-3.46 (m, 2H), 3.43-3.32 (m, 1H) 3.21 (s, 3H), 3.06-2.83 (m, 2H), 2.70-2.59 (m, 1H), 2.26 (s, 3H), 2.05-1.96 (m, 3H), 1.96-1.91 (m, 1H), 1.90-1.79 (m, 4H), 1.79-1.71 (m, 1H), 1.63-1.44 (m, 3H) | 529.31 |
| 167 | 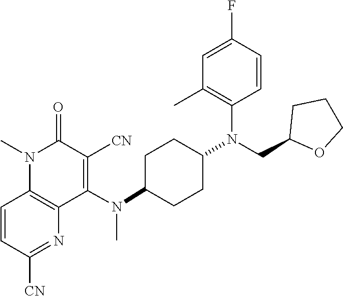 [SMILES: Cc1cc(F)ccc1N(C[C@H]1CCCO1)[C@H]1CC[C@H](N(C)c2c(C#N)c(=O)n(C)c3ccc(C#N)nc23)CC1] | (400 MHz, DMSO) δ 8.25 (d, J = 8.9 Hz, 1H), 8.13 (d, J = 8.9 Hz, 1H), 7.25 (dd, J = 8.8, 5.6 Hz, 1H), 7.03 (dd, J = 9.8, 3.0 Hz, 1H), 6.94 (td, J = 8.5, 3.1 Hz, 1H), 4.35-4.25 (m, 1H), 3.70-3.62 (m, 1H), 3.53 (s, 3H), 3.51-3.45 (m, 2H), 3.20 (s, 3H), 3.19- 3.15 (m, 1H), 2.87-2.75 (m, 1H), 2.70-2.58 (m, 1H), 2.27 (s, 3H), 2.03-1.93 (m, 2H), 1.91-1.81 (m, 3H), 1.79-1.62 (m, 4H), 1.60-1.40 (m, 3H) | 529.33 |
| 168 | 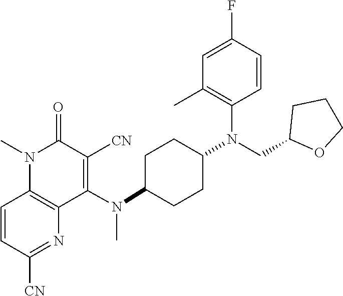 [SMILES: Cc1cc(F)ccc1N(C[C@@H]1CCCO1)[C@H]1CC[C@H](N(C)c2c(C#N)c(=O)n(C)c3ccc(C#N)nc23)CC1] | (400 MHz, DMSO) δ 8.25 (d, J = 8.9 Hz, 1H), 8.13 (d, J = 8.9 Hz, 1H), 7.25 (dd, J = 8.8,5.6 Hz, 1H), 7.03 (dd, J = 9.8, 3.0 Hz, 1H), 6.94 (td, J = 8.5, 3.1 Hz, 1H), 4.35-4.25 (m, 1H), 3.70-3.62 (m, 1H), 3.53 (s, 3H), 3.51-3.45 (m, 2H), 3.20 (s, 3H), 3.19- 3.15 (m, 1H), 2.87-2.75 (m, 1H), 2.70-2.58 (m, 1H), 2.27 (s, 3H), 2.03-1.93 (m, 2H), 1.91-1.81 (m, 3H), 1.79-1.62 (m, 4H), 1.60-1.40 (m, 3H) | 529.33 |
| 169 | 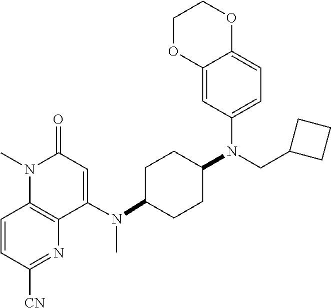 [SMILES: Cn1c(=O)cc(N(C)[C@H]2CC[C@@H](N(CC3CCC3)c3ccc4c(c3)OCCO4)CC2)c2nc(C#N)ccc21] | (300 MHz, DMSO) δ 8.13 (d, J = 9.0 Hz, 1H), 8.03 (d, J = 9.0 Hz, 1H), 6.77 (d, J = 8.3 Hz, 1H), 6.70- 6.58 (m, 2H), 5.89 (s, 1H), 4.41-4.27 (m, 1H), 4.21 (s, 4H), 3.51 (s, 3H), 2.93-2.81 (m, 5H), 2.22-1.93 (m, 3H), 1.91-1.69 (m, 6H), 1.67-1.45 (m, 4H), 1.45-1.29 (m, 2H) | 515.0 |
| 170 | 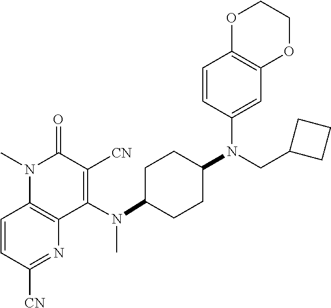 [SMILES: Cn1c(=O)c(C#N)c(N(C)[C@H]2CC[C@@H](N(CC3CCC3)c3ccc4c(c3)OCCO4)CC2)c2nc(C#N)ccc21] | 1H NMR (300 MHz, DMSO-d6) δ = 8.25 (d, J = 8.9 Hz, 1H), 8.18-8.09 (m, 1H), 6.77 (d, J = 8.2 Hz, 1H), 6.69-6.61 (m, 2H), 4.46-4.27 (m, J = 10.5, 10.5 Hz, 1H), 4.21 (s, 4H), 3.53 (s, 3H), 3.29 (s, 3H), 3.16-3.04 (m, 1H), 2.86 (d, J = 7.0 Hz, 2H), 2.29-2.02 (m, 3H), 1.90-1.54 (m, 10H), 1.54-1.22 (m, 2H) | 540.3 |
| 171 | 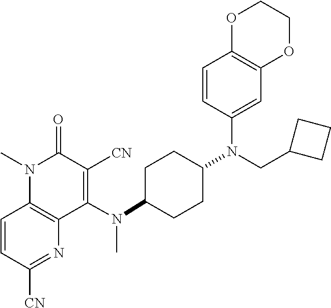 [SMILES: Cn1c(=O)c(C#N)c(N(C)[C@H]2CC[C@H](N(CC3CCC3)c3ccc4c(c3)OCCO4)CC2)c2nc(C#N)ccc21] | 1H NMR (300 MHz, DMSO-d6) δ = 8.26 (d, J = 8.9 Hz, 1H), 8.14 (d, J = 8.9 Hz, 1H), 6.68 (d, J = 8.3 Hz, 1H), 6.35-6.26 (m, 2H), 4.43-4.28 (m, 1H), 4.16 (d, J = 8.2 Hz, 4H), 3.54 (s, 3H), 3.46-3.39 (m, 1H), 3.27 (s, 3H), 3.00 (d, J = 6.3 Hz, 2H), 2.42-2.29 (m, 1H), 2.09-1.85 (m, 6H), 1.84-1.43 (m, 8H) | 539.9 |
| 172 | 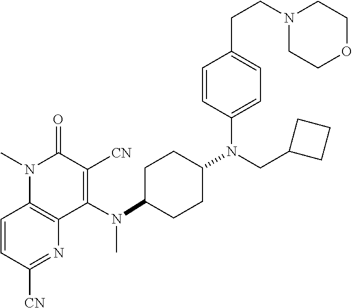 [SMILES: Cn1c(=O)c(C#N)c(N(C)[C@H]2CC[C@H](N(CC3CCC3)c3ccc(CCN4CCOCC4)cc3)CC2)c2nc(C#N)ccc21] | 1H NMR (300 MHz, DMSO-d6) δ = 8.27 (d, J = 8.8 Hz, 1H), 8.17-8.11 (m, 1H), 7.02 (d, J = 8.0 Hz, 2H), 6.69 (d, J = 8.3 Hz, 2H), 4.44-4.32 (m, 1H), 3.68-3.45 (m, 8H), 3.28 (s, 3H), 3.09 (d, J = 6.2 Hz, 2H), 2.68-2.57 (m, 2H), 2.48-2.35 (m, 3H), 2.12-1.88 (m, 5H), 1.86-1.51 (m, 6H), 1.26 (d, J = 6.6 Hz, 2H) | 594.6 |
| 173 | 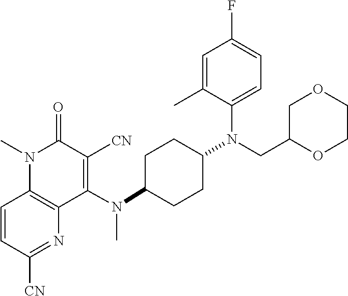 [SMILES: Cc1cc(F)ccc1N(CC1COCCO1)[C@H]1CC[C@H](N(C)c2c(C#N)c(=O)n(C)c3ccc(C#N)nc23)CC1] | 1H NMR (300 MHz, DMSO-d6) δ = 8.26 (d, J = 8.8 Hz, 1H), 8.13 (d, J = 8.9 Hz, 1H), 7.31-7.23 (m, 1H), 7.06 (d, J = 9.5 Hz, 1H), 6.97 (t, J = 8.4 Hz, 1H), 4.36-4.23 (m, 1H), 3.63 (d, J = 8.9 Hz, 2H), 3.59-3.47 (m, 4H), 3.46-3.28 (m, 2H), 3.28-3.08 (m, 6H), 2.99-2.78 (m, 1H), 2.71-2.54 (m, 1H), 2.25 (s, 3H), 1.98 (s, 2H), 1.93-1.71 (m, 4H), 1.65- 1.38 (m, 2H) | 545.5 |
| 174 | 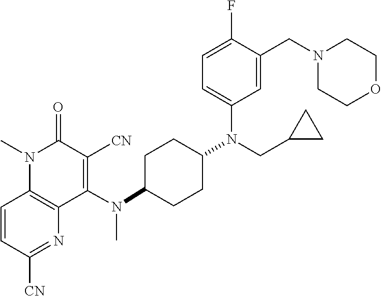 [SMILES: Cn1c(=O)c(C#N)c(N(C)[C@H]2CC[C@H](N(CC3CC3)c3ccc(F)c(CN4CCOCC4)c3)CC2)c2nc(C#N)ccc21] | 1H NMR (300 MHz, CDCl₃) δ 7.86 (d, J = 8.4 Hz, 1H), 7.73 (d, J = 8.9 Hz, 1H), 7.67-7.58 (m, 1H), 7.05-6.92 (m, 1H), 6.90-6.81 (m, 1H), 4.45-4.34 (br t, J = 12.15 Hz, 2 H) 4.13-4.26 (m, 1 H) 4.00 (br d, J = 13.02 Hz, 2 H) 3.69-3.85 (m, 2 H) 3.62- 3.69 (m, 3 H) 3.50 (br s, 2 H) 3.42 (br d, J = 11.10 Hz, 1 H) 3.36 (s, 1 H) 3.10 (br d, J = 5.04 Hz, 1 H) 2.85-3.05 (m, 2 H) 2.59 (br d, J = 2.02 Hz, 1 H) 2.27-2.43 (m, 1 H) 1.98-2.26 (m, 4 H) 1.76- 1.95 (m, 1 H) 1.44-1.73 (m, 18 H) 1.21-1.39 (m, 2 H) 0.82-1.04 (m, 1 H) 0.65-0.81 (m, 1 H) 0.58 (br d, J = 7.98 Hz, 1 H) 0.21-0.36 (m, 2 H) −0.01 (br s, 1 H). | |
| 175 | 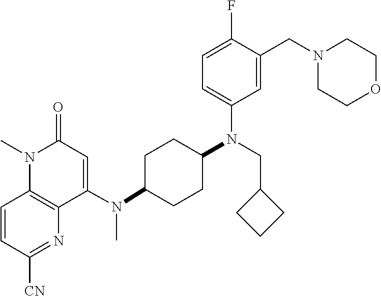 [SMILES: Cn1c(=O)cc(N(C)[C@H]2CC[C@@H](N(CC3CCC3)c3ccc(F)c(CN4CCOCC4)c3)CC2)c2nc(C#N)ccc21] | — | 573.6 |
| 176 | 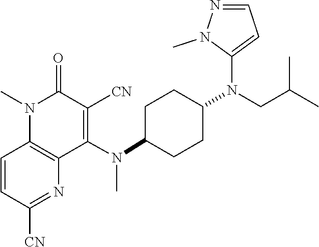 [SMILES: CC(C)CN(c1ccnn1C)[C@H]1CC[C@H](N(C)c2c(C#N)c(=O)n(C)c3ccc(C#N)nc23)CC1] | *Isolated as a mixture of diastereomers. (300 MHz, CDCl₃) δ ppm 7.88 (br d, J = 9.2 Hz, 1H), 7.78- 7.70 (m, 1H), 7.63 (br s, 1H), 6.07 (br s, 1H), 4.43- 4.28 (m, 1H), 3.94 (br s, 2H), 3.76 (s, 1H), 3.66 (s, 3H), 3.36 (s, 3H), 2.99-2.80 (m, 1H), 2.77 (d, J = 6.8 Hz, 2H), 2.26-2.14 (m, 3H) 2.10-2.00 (m, 3H), 1.93-1.75 (m, 4H), 1.70-1.40 (m, 6H), 0.88 (br d, J = 6.14 Hz, 6H). | 473.5 |
| 177 | 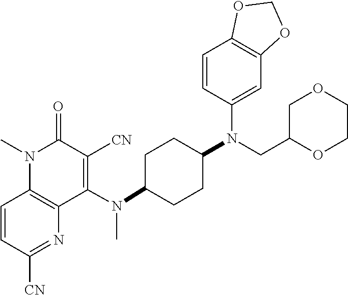 [SMILES: Cn1c(=O)c(C#N)c(N(C)[C@H]2CC[C@@H](N(CC3COCCO3)c3ccc4c(c3)OCO4)CC2)c2nc(C#N)ccc21] | 1H NMR (300 MHz, DMSO-d6) δ = 8.25 (d, J = 8.8 Hz, 1H), 8.14 (d, J = 8.6 Hz, 1H), 6.96 (s, 1H), 6.86 (d, J = 8.3 Hz, 1H), 6.74 (d, J = 8.0 Hz, 1H), 6.01 (s, 2H), 4.45-4.28 (m, 1H), 3.90 (d, J = 10.2 Hz, 1H), 3.65 (d, J = 7.7 Hz, 1H), 3.59 (d, J = 7.8 Hz, 1H), 3.54 (s, 3H), 3.44 (d, J = 8.0 Hz, 2H), 3.30 (s, 3H), 3.26-3.10 (m, 2H), 2.91 (d, J = 5.2 Hz, 2H), 2.23-1.94 (m, 4H), 1.76-1.54 (m, 4H), 1.53-1.28 (m, 2H) | 557.5 |
| 178 | 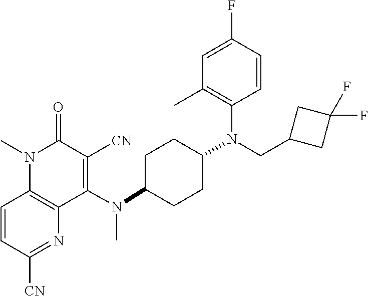 [SMILES: Cc1cc(F)ccc1N(CC1CC(F)(F)C1)[C@H]1CC[C@H](N(C)c2c(C#N)c(=O)n(C)c3ccc(C#N)nc23)CC1] | (400 MHz, DMSO-d6) δ 8.26 (d, J = 8.8 Hz, 1H), 8.13 (d, J = 9.0 Hz, 1H), 7.24 (dd, J = 8.8, 5.5 Hz, 1H), 7.05 (dd, J = 9.8, 3.1 Hz, 1H), 6.95 (td, J = 8.5, 3.2 Hz, 1H), 4.32-4.22 (m, 1H), 3.53 (s, 3H), 3.21 (s, 3H), 3.07 (bd, J = 7.1 Hz, 2H), 2.69-2.60 (m, 1H), 2.48-2.36 (m, 2H), 2.23 (s, 3H), 2.13 (tt, J = 14.4, 7.2 Hz, 2H), 1.99 (bd, J = 11.2 Hz, 2H), 1.92-1.77 (m, 5H), 1.56-1.42 (m, 2H) | 549.25 |
| 179 | 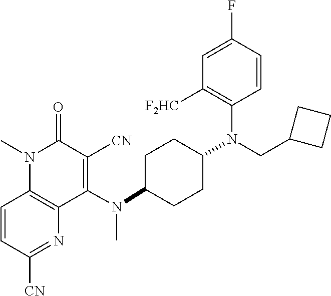 [SMILES: Cn1c(=O)c(C#N)c(N(C)[C@H]2CC[C@H](N(CC3CCC3)c3ccc(F)cc3C(F)F)CC2)c2nc(C#N)ccc21] | (300 MHz, DMSO-d6) δ 8.25 (d, J = 8.7 Hz, 1H), 8.13 (d, J = 8.9 Hz, 1H), 7.64-7.24 (m, 4H), 4.41- 4.28 (m, 1H), 3.53 (s, 3H), 3.26 (s, 3H), 2.97 (bd, J = 6.9 Hz, 2H), 2.37-2.20 (m, 1H), 2.21-1.35 (m, 15H) | 549.20 |
| 180 | 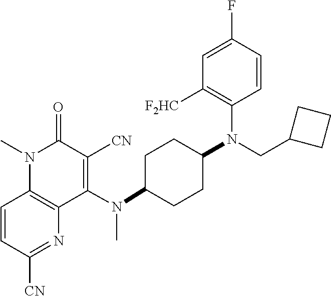 [SMILES: Cn1c(=O)c(C#N)c(N(C)[C@H]2CC[C@@H](N(CC3CCC3)c3ccc(F)cc3C(F)F)CC2)c2nc(C#N)ccc21] | (300 MHz, DMSO-d6) δ 8.25 (d, J = 8.9 Hz, 1H), 8.13 (d, J = 8.9 Hz, 1H), 7.58-7.49 (m, 1H), 7.41- 7.32 (m, 2H), 7.11 (t, J = 55.2 Hz, 1H), 4.43-4.30 (m, 1H), 3.52 (s, 3H), 3.20 (s, 3H), 3.02 (d, J = 7.4 Hz, 2H), 2.75-2.62 (m, 1H), 2.10-1.67 (m, 11H), 1.59-1.36 (m, 4H) | 549.25 |
| 181 | 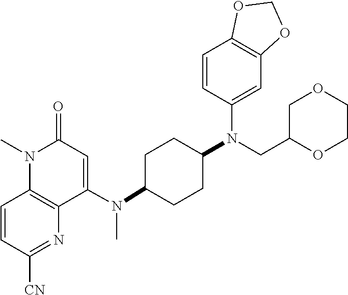 [SMILES: Cn1c(=O)cc(N(C)[C@H]2CC[C@@H](N(CC3COCCO3)c3ccc4c(c3)OCO4)CC2)c2nc(C#N)ccc21] | 1H NMR (300 MHz, DMSO-d6) δ = 8.13 (d, J = 8.8 Hz, 1H), 8.04 (d, J = 8.9 Hz, 1H), 6.95 (s, 1H), 6.86 (d, J = 8.1 Hz, 1H), 6.73 (d, J = 8.1 Hz, 1H), 6.01 (s, 2H), 5.90 (s, 1H), 4.42-4.24 (m, 1H), 3.88 (d, J = 10.7 Hz, 1H), 3.65 (d, J = 8.3 Hz, 1H), 3.59 (d, J = 8.1 Hz, 1H), 3.51 (s, 3H), 3.49-3.39 (m, 5H), 3.25-3.15 (m, 1H), 3.15-3.09 (m, 1H), 3.25- 3.04 (m, 1H), 2.90 (s, 3H), 2.10-1.88 (m, 4H), 1.72-1.50 (m, 2H), 1.50-1.22 (m, 4H) | 532.0 |
| 182 | 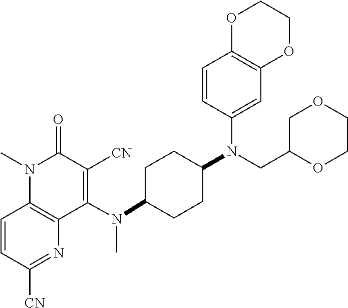 [SMILES: Cn1c(=O)c(C#N)c(N(C)[C@H]2CC[C@@H](N(CC3COCCO3)c3ccc4c(c3)OCCO4)CC2)c2nc(C#N)ccc21] | 1H NMR (300 MHz, DMSO-d6) δ = 8.25 (d, J = 9.4 Hz, 1H), 8.14 (d, J = 8.9 Hz, 1H), 6.84-6.68 (m, 3H), 4.42-4.30 (m, 1H), 4.30-4.16 (m, 3H), 3.86 (d, J = 10.6 Hz, 1H), 3.65 (d, J = 8.3 Hz, 1H), 3.59 (d, J = 8.1 Hz, 1H), 3.54 (s, 3H), 3.50-3.41 (m, J = 8.3 Hz, 2H), 3.29 (s, 3H), 3.25-3.11 (m, 2H), 2.91 (d, J = 5.5 Hz, 2H), 2.23-1.93 (m, 4H), 1.75-1.54 (m, 4H), 1.53-1.28 (m, 2H) | 570.9 |
| 183 | 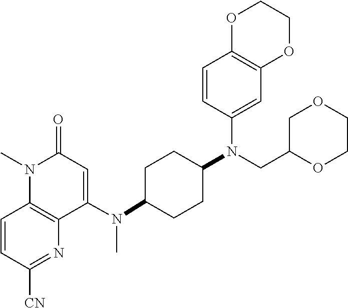 [SMILES: Cn1c(=O)cc(N(C)[C@H]2CC[C@@H](N(CC3COCCO3)c3ccc4c(c3)OCCO4)CC2)c2nc(C#N)ccc21] | 1H NMR (300 MHz, DMSO-d6) δ = 8.13 (d, J = 8.8 Hz, 1H), 8.03 (d, J = 8.6 Hz, 1H), 6.84-6.68 (m, 3H), 5.90 (s, 1H), 4.39-4.17 (m, 4H), 3.84 (d, J = 10.9 Hz, 1H), 3.65 (d, J = 8.3 Hz, 1H), 3.59 (d, J = 8.3 Hz, 2H), 3.51 (s, 3H), 3.27-3.07 (m, 4H), 2.94- 2.84 (m, 4H), 2.10-1.86 (m, 4H), 1.72-1.52 (m, 2H), 1.51-1.22 (m, 4H) | 546.0 |
| 184 | 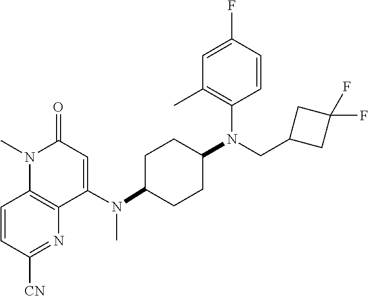 [SMILES: Cc1cc(F)ccc1N(CC1CC(F)(F)C1)[C@H]1CC[C@@H](N(C)c2cc(=O)n(C)c3ccc(C#N)nc23)CC1] | (400 MHz, DMSO-d6) δ 8.14 (d, J = 8.8 Hz, 1H), 8.03 (d, J = 8.9 Hz, 1H), 7.26 (dd, J = 8.9, 5.6 Hz, 1H), 7.07 (dd, J = 9.9, 3.1 Hz, 1H), 6.97 (td, J = 8.5, 3.1 Hz, 1H), 5.89 (s, 1H), 4.38-4.27 (m, 1H), 3.51 (s, 3H), 3.37 (s, 1H), 3.03 (br s, 2H), 2.86 (s, 3H), 2.49-2.43 (m, 2H), 2.41 (s, 3H), 2.20-1.87 (m, 5H), 1.80-1.28 (m, 6H) | 524.25 |
| 185 | 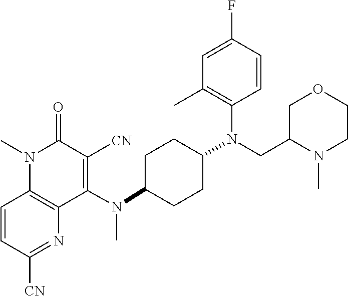 [SMILES: Cc1cc(F)ccc1N(CC1COCCN1C)[C@H]1CC[C@H](N(C)c2c(C#N)c(=O)n(C)c3ccc(C#N)nc23)CC1] | (400 MHz, DMSO-d6) δ 8.26 (d, J = 8.8 Hz, 1H), 8.16-8.11 (m, 1H), 7.26 (br s, 1H), 7.07 (dd, J = 9.8, 3.1 Hz, 1H), 6.96 (td, J = 8.5, 3.2 Hz, 1H), 4.34-4.27 (m, 1H), 3.75-3.57 (m, 2H), 3.53 (s, 3H), 3.45-3.35 (m, 2H), 3.20 (s, 3H), 3.10 (br s, 1H), 2.70-2.53 (m, 3H), 2.26 (s, 3H), 2.22 (br s, 3H), 2.06-1.93 (m, 3H), 1.92-1.68 (m, 5H), 1.68-1.46 (m, 2H) | 558.25 |
| 186 | 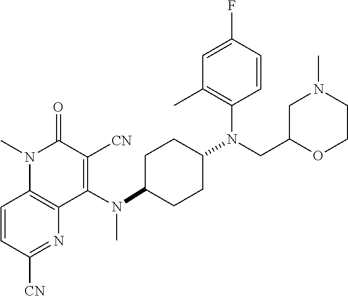 [SMILES: Cc1cc(F)ccc1N(CC1CN(C)CCO1)[C@H]1CC[C@H](N(C)c2c(C#N)c(=O)n(C)c3ccc(C#N)nc23)CC1] | (400 MHz, DMSO-d6) δ 10.03-9.75 (m, 1H), 8.26 (d, J = 8.9 Hz, 1H), 8.14 (d, J = 8.9 Hz, 1H), 7.29 (dd, J = 8.9, 5.5 Hz, 1H), 7.08 (dd, J = 9.8,3.0 Hz, 1H), 6.98 (td, J = 8.6, 3.1 Hz, 1H), 4.32-4.22 (m, 1H), 3.97 (dd, J = 12.9, 3.5 Hz, 1H), 3.53 (s, 3H), 3.52-3.44 (m, 1H), 3.41-3.23 (m, 4H), 3.21 (s, 3H), 3.00-2.89 (m, 2H), 2.85-2.64 (m, 5H), 2.29 (s, 3H), 2.02-1.94 (m, 2H), 1.93-1.73 (m, 4H), 1.59-1.38 (m, 2H) | 558.25 |
| 187 | 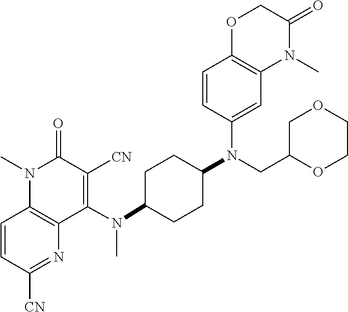 [SMILES: CN1C(=O)COc2ccc(N(CC3COCCO3)[C@H]3CC[C@@H](N(C)c4c(C#N)c(=O)n(C)c5ccc(C#N)nc45)CC3)cc21] | 1H NMR (300 MHz, DMSO-d6) δ = 8.25 (d, J = 8.9 Hz, 1H), 8.14 (d, J = 9.0 Hz, 1H), 7.00 (s, 1H), 6.95-6.95 (m, 1H), 6.94 (d, J = 8.2 Hz, 1H), 6.88 (d, J = 8.6 Hz, 1H), 4.62 (s, 2H), 4.44-4.31 (m, 1H), 3.85 (d, J = 10.8 Hz, 1H), 3.71-3.63 (m, 2H), 3.58 (s, 3H), 3.54-3.53 (m, 3H), 3.32-3.31 (m, 4H), 3.26-3.15 (m, 5H), 3.01 (d, J = 5.0 Hz, 2H), 2.18- 2.06 (m, 2H), 2.05-1.92 (m, 2H), 1.77-1.56 (m, 2H), 1.56-1.35 (m, 2H) | 598.0 |
| 188 | 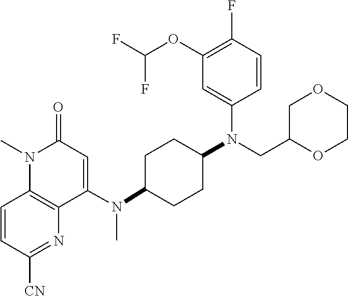 [SMILES: Cn1c(=O)cc(N(C)[C@H]2CC[C@@H](N(CC3COCCO3)c3ccc(F)c(OC(F)F)c3)CC2)c2nc(C#N)ccc21] | (400 MHz, DMSO-d6) δ 8.15 (d, J = 8.8 Hz, 1H), 8.05 (d, J = 8.9 Hz, 1H), 7.24 (t, J = 73.6 Hz, 1H), 7.33-7.26 (m, 1H), 7.15-7.10 (m, 1H), 7.08-7.02 (m, 1H), 5.96 (s, 1H), 4.33-4.24 (m, 1H), 3.76-3.56 (m, 3H), 3.52 (s, 3H), 3.48-3.37 (m, 4H), 3.19 (t, J = 10.6 Hz, 1H), 3.09 (d, J = 5.9 Hz, 2H), 2.89 (s, 3H), 2.03-1.90 (m, 3H), 1.80-1.71 (m, 1H), 1.65- 1.38 (m, 4H) | 572.15 |
| 189 | 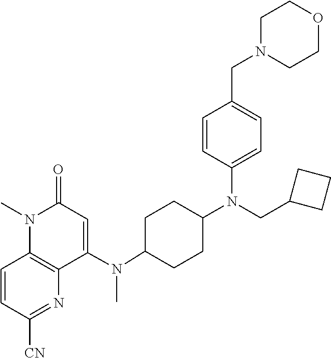 [SMILES: CN(c1cc(=O)n(C)c2ccc(C#N)nc12)C1CCC(N(CC2CCC2)c2ccc(CN3CCOCC3)cc2)CC1] | 1H NMR (300 MHz, DMSO-d6) δ = 8.16 (d, J = 9.1 Hz, 1H), 8.06 (d, J = 9.3 Hz, 1H), 7.07 (d, J = 8.2 Hz, 2H), 6.69 (d, J = 7.9 Hz, 2H), 5.90 (s, 1H), 4.45-4.29 (m, 1H), 3.68-3.48 (m, 8H), 3.11 (d, J = 5.5 Hz, 2H), 2.88 (s, 3H), 2.46-2.36 (m, 1H), 2.31 (s, 4H), 2.00-1.87 (m, 5H), 1.81-1.67 (m, 5H), 1.63-1.47 (m, 2H) | 555.7 |
| 190 | 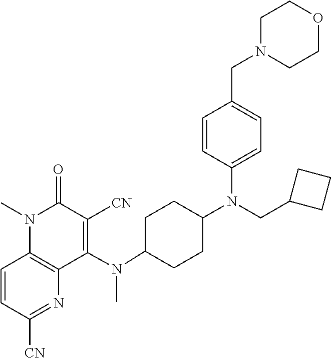 [SMILES: CN(c1c(C#N)c(=O)n(C)c2ccc(C#N)nc12)C1CCC(N(CC2CCC2)c2ccc(CN3CCOCC3)cc2)CC1] | 1H NMR (300 MHz, DMSO-d6) δ = 8.27 (d, J = 8.9 Hz, 1H), 8.15 (d, J = 8.5 Hz, 1H), 7.08 (d, J = 7.8 Hz, 2H), 6.71 (d, J = 7.7 Hz, 2H), 4.49-4.31 (m, 1H), 3.71-3.51 (m, 8H), 3.30-3.24 (m, 3H), 3.11 (d, J = 5.6 Hz, 2H), 2.47-2.36 (m, 1H), 2.32 (s, 4H), 2.14-1.87 (m, 6H), 1.85-1.52 (m, 6H), 1.84-1.49 (m, 7H) | 580.6 |
| 191 | 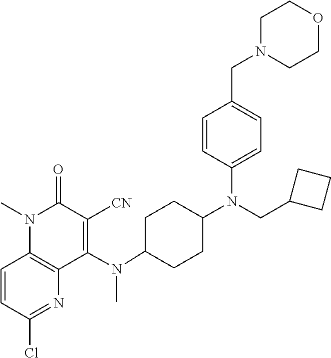 [SMILES: CN(c1c(C#N)c(=O)n(C)c2ccc(Cl)nc12)C1CCC(N(CC2CCC2)c2ccc(CN3CCOCC3)cc2)CC1] | 1H NMR (300 MHz, DMSO-d6) δ = 8.31 (d, J = 8.4 Hz, 1H), 8.16-8.07 (m, 2H), 7.09-6.87 (m, 3H), 4.56-4.34 (m, 1H), 3.67-3.47 (m, 8H), 3.06 (d, J = 5.4 Hz, 2H), 2.18-2.05 (m, 2H), 2.03-1.92 (m, 2H), 1.91-1.69 (m, 4H), 1.67-1.33 (m, 4H), 1.02-0.81 (m, 1H), 0.46 (d, J = 8.1 Hz, 2H), 0.22 (d, J = 4.1 Hz, 2H) | 589.6 |
| 192 | 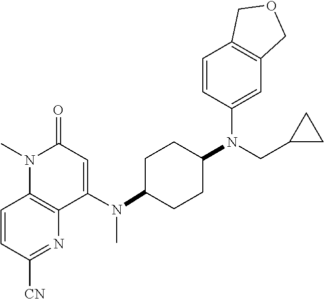 [SMILES: Cn1c(=O)cc(N(C)[C@H]2CC[C@@H](N(CC3CC3)c3ccc4c(c3)COC4)CC2)c2nc(C#N)ccc21] | 1H NMR (300 MHz, DMSO-d6) δ = 8.15 (d, J = 8.8 Hz, 1H), 8.05 (d, J = 8.9 Hz, 1H), 7.20 (d, J = 8.2 Hz, 1H), 7.14-7.05 (m, 2H), 5.94 (s, 1H), 4.96 (s, 4H), 4.37-4.24 (m, 1H), 3.61-3.43 (m, 6H), 2.95 (d, J = 6.1 Hz, 2H), 2.89 (s, 3H), 2.10-1.87 (m, 4H), 1.60-1.38 (m, 4H), 0.78-0.66 (m, 1H), 0.37- 0.27 (m, J = 7.5 Hz, 2H), 0.08-−0.08 (m, 2H) | 484.1 |
| 193 | 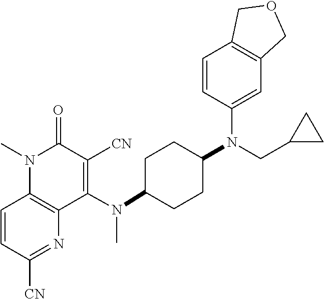 [SMILES: Cn1c(=O)c(C#N)c(N(C)[C@H]2CC[C@@H](N(CC3CC3)c3ccc4c(c3)COC4)CC2)c2nc(C#N)ccc21] | 1H NMR (300 MHz, DMSO-d6) δ = 8.27 (d, J = 9.0 Hz, 1H), 8.16 (d, J = 8.9 Hz, 1H), 7.19 (d, J = 7.7 Hz, 1H), 7.12-7.03 (m, 2H), 4.96 (d, J = 4.5 Hz, 4H), 4.46-4.34 (m, 1H), 3.63-3.44 (m, 4H), 3.28 (s, 3H), 2.95 (d, J = 6.0 Hz, 2H), 2.23-2.03 (m, 2H), 1.95-1.82 (m, J = 12.0 Hz, 2H), 1.81-1.62 (m, 2H), 1.62-1.40 (m, 2H), 0.82-0.67 (m, 1H), 0.33 (d, J = 7.6 Hz, 2H), -0.02 (d, J = 4.5 Hz, 2H) | 509.1 |
| 194 | 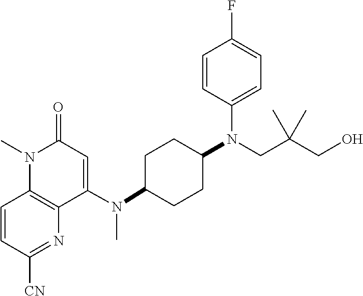 [SMILES: Cn1c(=O)cc(N(C)[C@H]2CC[C@@H](N(CC(C)(C)CO)c3ccc(F)cc3)CC2)c2nc(C#N)ccc21] | (400 MHz, DMSO-d6) δ 8.13 (d, J = 8.8 Hz, 1H), 8.03 (d, J = 8.9 Hz, 1H), 7.37-7.31 (m, 2H), 7.11 (t, J = 8.8 Hz, 2H), 5.92 (s, 1H), 4.44 (t, J = 5.1 Hz, 1H), 4.32-4.25 (m, 1H), 3.51 (s, 3H), 3.11-3.07 (m, 1H), 3.30 (d, J = 5.2 Hz, 2H), 2.97 (s, 2H), 2.89 (s, 3H), 2.10-1.98 (m, 2H), 1.81 (bd, J = 13.8 Hz, 2H), 1.53-1.35 (m, 4H), 0.66 (s, 6H) | 492.20 |
| 195 | 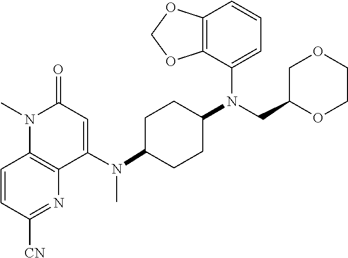 [SMILES: Cn1c(=O)cc(N(C)[C@H]2CC[C@@H](N(C[C@H]3COCCO3)c3cccc4c3OCO4)CC2)c2nc(C#N)ccc21] | (300 MHz, DMSO-d6) δ 8.14 (d, J = 8.8 Hz, 1H), 8.03 (d, J = 8.9 Hz, 1H), 6.86-6.78 (m, 2H), 6.73 (dd, J = 5.8, 3.1 Hz, 1H), 6.00 (d, J = 4.7 Hz, 2H), 5.88 (s, 1H), 4.39-4.27 (m, 1H), 3.88 (dd, J = 11.5, 2.1 Hz, 1H), 3.61 (dd, J = 18.5, 9.4 Hz, 2H), 3.59 (s, 3H), 3.47-3.35 (m, 4H), 3.26-3.14 (m, 1H), 3.02 (d, J = 6.2 Hz, 2H), 2.86 (s, 3H), 2.11-1.87 (m, 3H), 1.80-1.67 (m, 1H), 1.64-1.31 (m, 4H) | 532.25 |
| 196 | 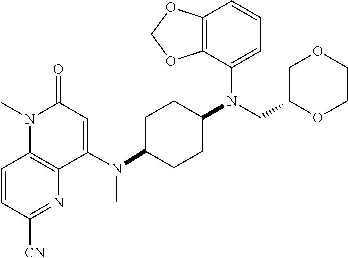 [SMILES: Cn1c(=O)cc(N(C)[C@H]2CC[C@@H](N(C[C@@H]3COCCO3)c3cccc4c3OCO4)CC2)c2nc(C#N)ccc21] | (300 MHz, DMSO-d6) δ 8.14 (d, J = 8.8 Hz, 1H), 8.03 (d, J = 8.9 Hz, 1H), 6.86-6.78 (m, 2H), 6.73 (dd, J = 5.8, 3.1 Hz, 1H), 6.00 (d, J = 4.7 Hz, 2H), 5.88 (s, 1H), 4.39-4.27 (m, 1H), 3.88 (dd, J = 11.5, 2.1 Hz, 1H), 3.61 (dd, J = 18.5, 9.4 Hz, 2H), 3.59 (s, 3H), 3.47-3.35 (m, 4H), 3.26-3.14 (m, 1H), 3.02 (d, J = 6.2 Hz, 2H), 2.86 (s, 3H), 2.11-1.87 (m, 3H), 1.80-1.67 (m, 1H), 1.64-1.31 (m, 4H) | 532.25 |
| 197 | 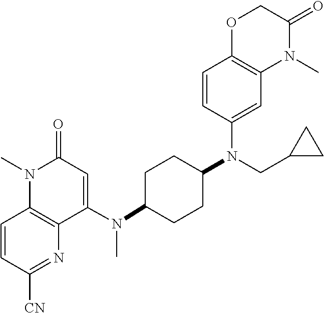 [SMILES: CN1C(=O)COc2ccc(N(CC3CC3)[C@H]3CC[C@@H](N(C)c4cc(=O)n(C)c5ccc(C#N)nc45)CC3)cc21] | 1H NMR (300 MHz, DMSO-d6) δ = 8.14 (d, J = 7.8 Hz, 1H), 8.04 (d, J = 8.6 Hz, 1H), 7.00-6.83 (m, 3H), 5.92 (s, 1H), 4.61 (s, 2H), 4.40-4.26 (m, 1H), 3.51 (s, 3H), 3.44-3.42 (m, 1H), 3.29-3.21 (m, 3H), 3.01-2.79 (m, 5H), 2.11-1.86 (m, 4H), 1.59-1.34 (m, 4H), 0.76-0.64 (m, 1H), 0.34 (d, J = 7.5 Hz, 2H), 0.01-−0.08 (m, 2H) | 527.6 |
| 198 | 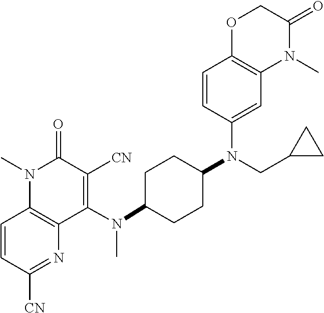 [SMILES: CN1C(=O)COc2ccc(N(CC3CC3)[C@H]3CC[C@@H](N(C)c4c(C#N)c(=O)n(C)c5ccc(C#N)nc45)CC3)cc21] | 1H NMR (300 MHz, DMSO-d6) δ = 8.25 (d, J = 9.5 Hz, 1H), 8.14 (d, J = 8.7 Hz, 1H), 6.94 (d, J = 9.5 Hz, 2H), 6.86 (d, J = 8.7 Hz, 1H), 4.61 (s, 2H), 4.47-4.32 (m, 1H), 3.54 (s, 3H), 3.31-3.25 (m, J = 5.0 Hz, 6H), 2.89 (d, J = 6.1 Hz, 2H), 2.27-2.06 (m, 2H), 1.98-1.81 (m, 2H), 1.74-1.58 (m, J = 11.1 Hz, 2H), 1.58-1.37 (m, J = 12.2, 12.2 Hz, 2H), 0.78- 0.65 (m, 1H), 0.34 (d, J = 7.4 Hz, 2H), −0.02 (d, J = 4.2 Hz, 2H) | 552.6 |
| 199 | 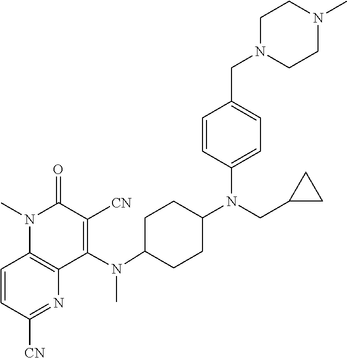 [SMILES: CN1CCN(Cc2ccc(N(CC3CC3)C3CCC(N(C)c4c(C#N)c(=O)n(C)c5ccc(C#N)nc45)CC3)cc2)CC1] | 1H NMR (300 MHz, DMSO-d6) δ = 8.27 (d, J = 8.7 Hz, 1H), 8.15 (d, J = 8.7 Hz, 1H), 7.08 (d, J = 7.9 Hz, 2H), 6.80 (d, J = 7.9 Hz, 2H), 4.47-4.33 (m, 1H), 3.78-3.67 (m, 1H), 3.54 (s, 3H), 3.33-3.29 (m, J = 5.7 Hz, 6H), 3.03 (d, J = 4.9 Hz, 2H), 2.45-2.21 (m, 7H), 2.16 (s, 3H), 2.11-1.99 (m, 4H), 1.90-1.76 (m, J = 11.1 Hz, 2H), 1.72-1.52 (m, 2H), 1.00-0.82 (m, J = 5.2 Hz, 1H), 0.47 (d, J = 7.6 Hz, 2H), 0.27- 0.19 (m, J = 4.3 Hz, 2H) | 579.7 |
| 200 | 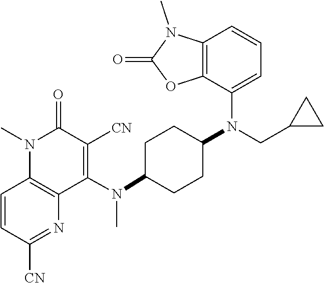 [SMILES: Cn1c(=O)c(C#N)c(N(C)[C@H]2CC[C@@H](N(CC3CC3)c3cccc4c3oc(=O)n4C)CC2)c2nc(C#N)ccc21] | (300 MHz, Chloroform-d) 8 7.83 (d, J = 8.8 Hz, 1H), 7.70 (d, J = 8.8 Hz, 1H), 7.18-7.08 (m, 1H), 7.05-6.95 (m, 1H), 6.78-6.69 (m, 1H), 4.54-4.42 (m, 1H), 3.77 (s, 1H), 3.62 (s, 3H), 3.42 (s, 6H), 3.10-3.00 (m, 2H), 2.31-2.04 (m, 4H), 1.75-1.70 (m, 2H), 1.67-1.60 (m, 1H), 0.92-0.74 (m, 2H), 0.36-0.25 (m, 2H), −0.14 (br s, 2H) | 538.30 |
| 201 | 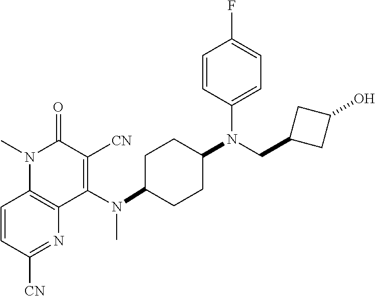 | 1H NMR (300 MHz, DMSO-d6) δ = 8.25 (d, J = 8.7 Hz, 1H), 8.15 (d, J = 8.9 Hz, 1H), 7.23-7.07 (m, 4H), 4.91-4.81 (m, 1H), 4.46-4.32 (m, 1H), 4.14 (d, J = 1.4 Hz, 1H), 3.54 (s, 4H), 3.22 (s, 3H), 2.94 (d, J = 6.8 Hz, 2H), 2.24-2.05 (m, 2H), 1.99-1.60 (m, 9H), 1.53-1.37 (m, 2H) | 515.1 |
| 202 | 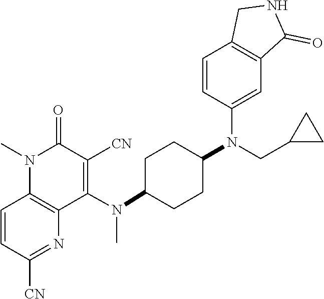 [SMILES: Cn1c(=O)c(C#N)c(N(C)[C@H]2CC[C@@H](N(CC3CC3)c3ccc4c(c3)C(=O)NC4)CC2)c2nc(C#N)ccc21] | (400 MHz, DMSO-d6) δ 8.37 (d, J = 8.8 Hz, 1H), 8.17 (d, J = 8.8 Hz, 1H), 7.86 (d, J = 8.3 Hz, 1H), 7.38 (d, J = 8.4 Hz, 1H), 7.15 (dd, J = 8.5, 2.5 Hz, 1H), 7.09 (d, J = 2.4 Hz, 1H), 4.67-4.60 (m, 1H), 4.32 (s, 2H), 3.90-3.81 (m, 1H), 3.52 (s, 3H), 3.16 (d, J = 5.8 Hz, 2H), 3.05 (s, 3H), 2.17-2.09 (m, 2H), 2.00-1.90 (m, 2H), 1.77-1.63 (m, 4H), 0.95- 0.88 (m, 1H), 0.53-0.49 (m, 2H), 0.28-0.23 (m, 2H) | 522.30 |
| 203 | 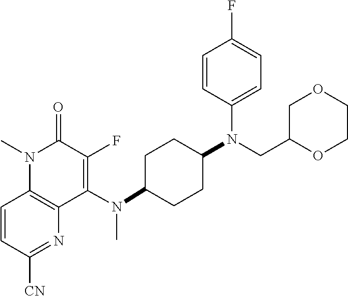 [SMILES: Cn1c(=O)c(F)c(N(C)[C@H]2CC[C@@H](N(CC3COCCO3)c3ccc(F)cc3)CC2)c2nc(C#N)ccc21] | (300 MHz, Chloroform-d) δ 7.80 (d, J = 8.7 Hz, 1H), 7.71 (d, J = 8.7 Hz, 1H), 7.05-6.93 (m, 4H), 3.88-3.76 (m, 2H), 3.75-3.71 (m, 4H), 3.69-3.56 (m, 3H), 3.54-3.45 (m, 1H), 3.33-3.25 (m, 2H), 3.13-2.98 (m, 5H), 2.17-2.01 (m, 2H), 1.94-1.81 (m, 1H), 1.80-1.67 (m, 1H), 1.66-1.60 (m, 2H), 1.52-1.38 (m, 2H) | 524.30 |
| 204 | 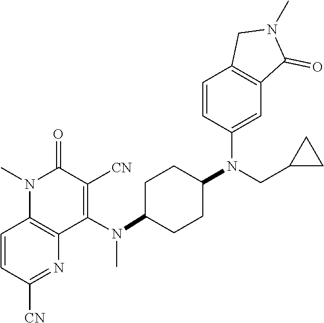 [SMILES: CN1Cc2ccc(N(CC3CC3)[C@H]3CC[C@@H](N(C)c4c(C#N)c(=O)n(C)c5ccc(C#N)nc45)CC3)cc2C1=O] | (300 MHz, DMSO-d6) δ 8.27 (d, J = 8.9 Hz, 1H), 8.16 (d, J = 9.0 Hz, 1H), 7.45 (d, J = 8.1 Hz, 1H), 7.38-7.30 (m, 2H), 4.47-4.35 (m, 3H), 3.63 (br s, 1H), 3.55 (s, 3H), 3.27 (s, 3H), 3.10-3.00 (m, 5H), 2.20-2.05 (m, 2H), 1.91-1.79 (m, 2H), 1.75-1.65 (m, 2H), 1.57-1.45 (m, 2H), 0.80-0.70 (m, 1H), 0.36-0.28 (m, 2H), (−)0.02-(−)0.17 (m, 2H) | 536.35 |
| 205 | 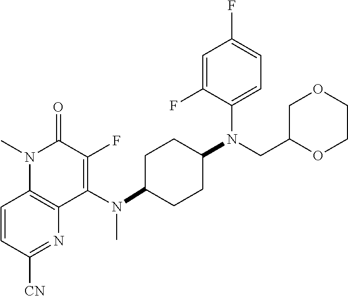 [SMILES: Cn1c(=O)c(F)c(N(C)[C@H]2CC[C@@H](N(CC3COCCO3)c3ccc(F)cc3F)CC2)c2nc(C#N)ccc21] | (300 MHz, Chloroform-d) 8 7.77 (d, J = 8.7 Hz, 1H), 7.69 (d, J = 8.7 Hz, 1H), 7.24-7.16 (m, 1H), 6.88-6.78 (m, 2H), 3.88 (dd, J = 11.3, 2.4 Hz, 1H), 3.82-3.56 (m, 8H), 3.55-3.44 (m, 1H), 3.34-3.27 (m, 2H), 3.12-3.04 (m, 4H), 2.96 (dd, J = 13.7, 6.9 Hz, 1H), 2.15-1.91 (m, 3H), 1.81-1.63 (m, 3H), 1.53-1.37 (m, 2H) | 542.30 |
| 206 | 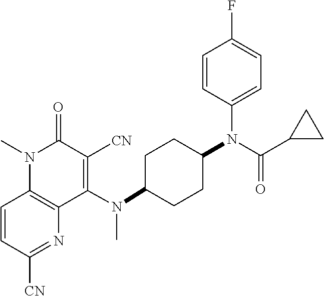 [SMILES: Cn1c(=O)c(C#N)c(N(C)[C@H]2CC[C@@H](N(C(=O)C3CC3)c3ccc(F)cc3)CC2)c2nc(C#N)ccc21] | 1H NMR (300 MHz, DMSO-d6) δ = 8.24 (d, J = 8.9 Hz, 1H), 8.14 (d, J = 9.1 Hz, 1H), 7.50-7.42 (m, 2H), 7.35-7.25 (m, 2H), 4.57-4.47 (m, 1H), 4.33- 4.22 (m, 1H), 3.53 (s, 3H), 3.00 (s, 3H), 1.80-1.59 (m, 8H), 1.11-0.99 (m, 1H), 0.78-0.69 (m, 2H), 0.59-0.49 (m, J = 5.3 Hz, 2H) | 498.8 |
| 207 | 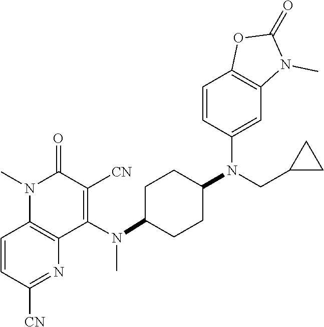 [SMILES: Cn1c(=O)oc2ccc(N(CC3CC3)[C@H]3CC[C@@H](N(C)c4c(C#N)c(=O)n(C)c5ccc(C#N)nc45)CC3)cc21] | (300 MHz, DMSO-d6) δ 8.25 (d, J = 8.9 Hz, 1H), 8.15 (d, J = 8.9 Hz, 1H), 7.23 (d, J = 8.5 Hz, 1H), 7.14 (d, J = 2.1 Hz, 1H), 6.96 (dd, J = 8.6, 2.1 Hz, 1H), 4.45-4.33 (m, 1H), 3.54 (s, 3H), 3.47 (br s, 1H), 3.35 (s, 3H), 3.31 (s, 3H), 2.92 (d, J = 6.5 Hz, 2H), 2.22-2.08 (m, 2H), 1.91 (bd, J = 13.5 Hz, 2H), 1.67 (bd, J = 11.6 Hz, 2H), 1.55-1.40 (m, 2H), 0.78-0.64 (m, 1H), 0.37-0.27 (m, 2H), (−)0.02- (−)0.08 (m, 2H) | 538.20 |
| 208 | 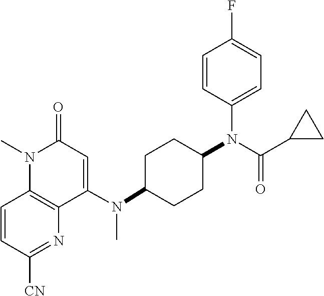 [SMILES: Cn1c(=O)cc(N(C)[C@H]2CC[C@@H](N(C(=O)C3CC3)c3ccc(F)cc3)CC2)c2nc(C#N)ccc21] | 1H NMR (300 MHz, DMSO-d6) δ = 8.13 (d, J = 8.7 Hz, 1H), 8.03 (d, J = 8.8 Hz, 1H), 7.56-7.47 (m, 2H), 7.40-7.29 (m, 2H), 5.90 (s, 1H), 4.57-4.46 (m, 1H), 4.17-4.03 (m, 1H), 3.50 (s, 3H), 2.60 (s, 3H), 1.95-1.81 (m, 2H), 1.74-1.59 (m, 2H), 1.58-1.45 (m, J = 3.0 Hz, 4H), 1.13-1.01 (m, 1H), 0.77-0.69 (m, 2H), 0.60-0.48 (m, J = 3.7 Hz, 2H) | 474.1 |
| 209 | 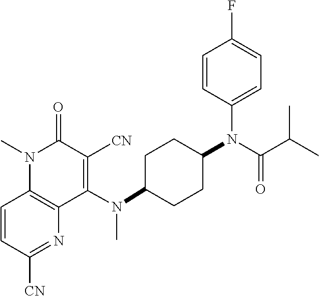 [SMILES: CC(C)C(=O)N(c1ccc(F)cc1)[C@H]1CC[C@@H](N(C)c2c(C#N)c(=O)n(C)c3ccc(C#N)nc23)CC1] | 1H NMR (300 MHz, DMSO-d6) δ = 8.24 (d, J = 8.6 Hz, 1H), 8.13 (d, J = 9.1 Hz, 1H), 7.49-7.37 (m, 2H), 7.33-7.24 (m, 2H), 4.56-4.45 (m, 1H), 4.32- 4.22 (m, 1H), 3.53 (s, 3H), 2.99 (s, 3H), 2.24-2.08 (m, 1H), 1.66 (s, 8H), 0.89 (d, J = 6.5 Hz, 6H) | 501.5 |
| 210 | 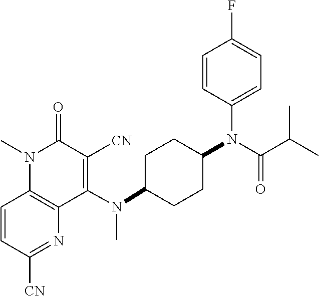 [SMILES: CC(C)C(=O)N(c1ccc(F)cc1)[C@H]1CC[C@@H](N(C)c2c(C#N)c(=O)n(C)c3ccc(C#N)nc23)CC1] | 1H NMR (300 MHz, DMSO-d6) δ = 8.12 (d, J = 8.8 Hz, 1H), 8.02 (d, J = 8.8 Hz, 1H), 7.53-7.43 (m, 2H), 7.38-7.29 (m, 2H), 5.90 (s, 1H), 4.55-4.44 (m, 1H), 4.15-4.03 (m, 1H), 3.50 (s, 3H), 2.59 (s, 3H), 2.51 (s, 4H), 2.24-2.12 (m, 1H), 1.91-1.75 (m, 2H), 1.72-1.57 (m, 2H), 1.57-1.42 (m, 4H), 0.89 (d, J = 6.5 Hz, 6H) | 476.1 |
| 211 | 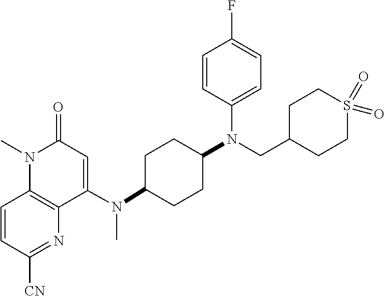 [SMILES: Cn1c(=O)cc(N(C)[C@H]2CC[C@@H](N(CC3CCS(=O)(=O)CC3)c3ccc(F)cc3)CC2)c2nc(C#N)ccc21] | (300 MHz, DMSO-d6) δ 8.14 (d, J = 9.0 Hz, 1H), 8.05 (d, J = 9.0 Hz, 1H), 7.35-7.25 (m, 2H), 7.22- 7.11 (m, 2H), 5.93 (s, 1H), 4.39-4.24 (m, 1H), 3.52 (s, 3H), 3.14 (br s, 1H), 3.05-2.95 (m, 3H), 2.91 (s, 3H), 2.83 (d, J = 6.7 Hz, 2H), 2.15 (d, J = 13.3 Hz, 2H), 2.07-1.92 (m, 2H), 1.82 (d, J = 12.6 Hz, 2H), 1.68-1.47 (m, 4H), 1.46-1.31 (m, 4H) | — |
| 212 | 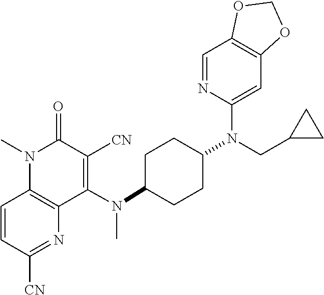 [SMILES: Cn1c(=O)c(C#N)c(N(C)[C@H]2CC[C@H](N(CC3CC3)c3cc4c(cn3)OCO4)CC2)c2nc(C#N)ccc21] | (300 MHz, DMSO-d6) δ 8.27 (d, J = 8.9 Hz, 1H), 8.14 (d, J = 9.0 Hz, 1H), 7.65 (s, 1H), 6.43 (s, 1H), 5.97 (s, 2H), 4.47-4.32 (m, 1H), 4.31-4.19 (m, 1H), 3.54 (s, 3H), 3.29 (s, 3H), 3.15 (d, J = 6.0 Hz, 2H), 2.09-1.95 (m, 4H), 1.83-1.73 (m, 2H), 1.72-1.57 (m, 2H), 1.01-0.89 (m, 1H), 0.48-0.40 (m, 2H), 0.29-0.22 (m, 2H) | 512.25 |
| 213 | 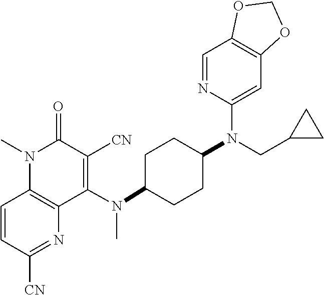 [SMILES: Cn1c(=O)c(C#N)c(N(C)[C@H]2CC[C@@H](N(CC3CC3)c3cc4c(cn3)OCO4)CC2)c2nc(C#N)ccc21] | (300 MHz, DMSO-d6) δ 8.30 (d, J = 8.9 Hz, 1H), 8.22 (d, J = 9.0 Hz, 1H), 7.64 (s, 1H), 6.44 (s, 1H), 5.97 (s, 2H), 4.41 (br s, 1H), 4.30-4.18 (m, 1H), 3.58 (s, 3H), 3.25 (s, 3H), 3.15 (d, J = 6.1 Hz, 2H), 2.18-2.06 (m, 2H), 1.80-1.53 (m, 4H), 1.52-1.41 (m, 2H), 0.95-0.82 (m, 1H), 0.39-0.31 (m, 2H), 0.15-0.08 (m, 2H) | 512.30 |
| 214 | 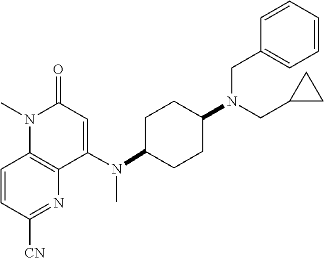 [SMILES: Cn1c(=O)cc(N(C)[C@H]2CC[C@@H](N(Cc3ccccc3)CC3CC3)CC2)c2nc(C#N)ccc21] | (300 MHz, CDCl3) δ 7.80 (d, J = 8.8 Hz, 1H), 7.70 (d, J = 8.7 Hz, 1H), 7.50 (d, J = 6.9 Hz, 2H), 7.40- 7.30 (m, 3H), 6.03 (s, 1H), 4.34 (t, J = 11.1 Hz, 1H), 3.90 (s, 2H), 3.64 (s, 3H), 3.00-2.81 (m, 4H), 2.56 (d, J = 5.5 Hz, 2H), 2.23-1.99 (m, 4H), 1.79- 1.43 (m, 4H), 0.99 (br s, 1H), 0.56 (d, J = 7.2 Hz, 2H), 0.15 (d, J = 4.0 Hz, 2H) | — |

### BIOLOGICAL ASSAYS

### Example 215

### DGKα and DGKζ Biochemical Assays

Compounds of the present invention were prepared into 10 mM DMSO solution and 10 nL of stock was transferred into 384 plates (Optiplate 384 plate) using Echo550. DMSO was used as high control, and ATP substrate buffer was used as a low control. A 1× enzyme assay buffer was prepared (Hepes, pH 7.0 25 mM, BSA 0.05%, Triton-X100 0.002%, CaCl2) 1 μM, MgCl2 10 mM, DTT 2 mM). The enzyme assay was performed by diluting enzyme DGKα (1 μg/μL DGKα, Carna12-101, SEQ ID NO: 3) or DGKζ (1μ/μL DGKζ, Carna 12-110, SEQ ID NO: 4) using 1× assay buffer. OAG (1-oleoyl-2-acetyl-sn-glycerol, 25 mg/ml, Avanti 8001000) and PS (10 mg/ml, Avanti 840032P) were mixed at the ratio of 1:2. A 1× substrate solution was prepared with 1× assay buffer by 100-fold dilution. The substrate solution was sonicated on ice for 1 min. The pure ATP was added to the substrate solution (DGKα:400 μM). 5 μL of the enzyme solution were added to the 384 well plate, and the plate was spun for 1 min at 1000 rpm and incubated for 30 mins at RT. 5 μL of 1× substrate solution were added to the 384 well plate, the plate was spun and then incubated for 45 mins at RT. 10 μL ADP-Glo detergent was added to stop the assay. After 60 mins at RT, 20 μL ADP-Glo Detection buffer was added as the final step. Plate was read after 45 min incubation at RT.

Data analysis was performed by calculating the 00 Inhibition using following the formula:

$$\% Inhibition = \frac{Hc - sample}{Hc - Lc} \cdot 100$$

High control (Hc): DMSO/DGKα(DGKζ)/Substrate/ATP/ADP-Glo
Low Control (Lc): ATP/ADP-Glo

The activity of the compounds of the invention are presented in Table 3, wherein:
“A” denotes an IC₅₀ of less than 10 nM;
“B” denotes an IC₅₀ of from 10 nM to less than 100 nM;
“C” denotes an IC₅₀ of from 100 nM to less than 1000 nM; and
“D” denotes an IC₅₀ of 1000 nM or more.

TABLE 3
Compound Activity

| | DGK α | DGK ζ |
|---|---|---|
| Cmpd. No. | IC₅₀ (nM) | IC₅₀ (nM) |
| 1A | C | D |
| 1B | B | C |
| 1C | D | D |
| 1D | C | D |
| 1E | B | D |
| 1F | B | D |
| 2A | B | D |
| 2B | C | D |
| 2C | C | D |
| 3A | A | D |
| 3B | A | D |
| 3C | B | D |
| 3D | B | C |
| 3E | B | D |
| 3F | B | C |
| 3G | B | D |
| 3H | C | B |
| 3I | B | C |
| 3J | B | C |
| 3K | B | C |
| 3L | B | C |
| 3M | B | C |
| 3N | B | D |
| 3O | C | D |
| 3Q | C | D |
| 3R | B | D |
| 3S | — | — |
| 3T | C | C |
| 3U | C | C |
| 3V | D | C |
| 3W | C | C |
| 3X | C | D |
| 3Y | D | D |
| 3Z | C | C |
| 3AA | C | D |
| 3BB | D | D |
| 3CC | C | D |
| 4A | D | D |
| 4B | D | D |
| 4C | D | D |
| 4D | D | D |
| 4E | D | D |
| 4F | D | D |
| 4G | D | D |
| 5 | C | D |
| 6 | B | D |
| 7 | B | D |
| 8 | B | D |
| 9 | D | D |
| 10 | A | C |
| 11 | B | C |
| 12 | C | D |
| 13 | B | D |
| 14 | D | D |
| 15 | C | D |
| 16 | A | C |
| 17 | D | D |
| 18 | C | C |
| 19 | C | C |
| 20 | B | D |
| 21 | B | D |
| 22 | D | D |
| 23 | B | C |
| 24 | B | D |
| 25 | D | D |
| 26 | B | C |
| 27 | C | C |
| 28 | B | C |
| 29 | C | D |
| 30 | A | B |
| 31 | C | C |
| 32 | B | D |
| 33 | C | C |
| 34 | C | D |
| 35 | A | D |
| 36 | D | D |
| 37 | D | C |
| 38 | C | C |
| 39 | B | C |
| 40 | B | D |
| 41 | D | D |
| 42 | A | C |
| 43 | A | C |
| 44 | C | D |
| 45 | B | C |
| 46 | C | B |
| 47 | B | D |
| 48 | C | D |
| 49 | A | C |
| 50 | A | C |
| 51 | A | C |
| 52 | A | B |
| 53 | B | C |
| 54 | C | C |
| 55 | A | B |
| 56 | B | D |
| 57 | A | B |
| 58 | C | D |
| 59 | C | C |
| 60 | C | B |
| 61 | C | B |
| 62 | D | C |
| 63 | A | C |
| 64 | B | B |
| 65 | B | B |
| 66 | B | C |
| 67 | B | C |
| 68 | A | B |
| 69 | B | B |
| 70 | D | C |
| 71 | D | B |
| 72 | D | C |
| 73 | B | B |
| 74 | D | B |
| 75 | B | D |
| 76 | C | C |
| 77 | D | C |
| 78 | C | B |
| 79 | D | C |
| 80 | D | D |
| 81 | B | B |
| 82 | B | C |
| 83 | C | C |
| 84 | B | C |
| 85 | A | D |
| 86 | C | C |
| 87 | C | C |
| 88 | D | D |
| 89 | D | D |
| 90 | D | D |
| 91 | A | B |
| 92 | B | C |
| 93 | C | D |
| 94 | C | C |
| 95 | D | C |
| 96 | A | D |
| 97 | A | B |
| 98 | C | C |
| 99 | A | B |
| 100 | A | D |
| 101 | D | B |
| 102 | A | C |
| 103 | D | D |
| 104 | D | D |
| 105 | D | C |
| 106 | C | C |
| 107 | D | C |
| 108 | B | C |
| 109 | B | B |
| 110 | B | C |
| 111 | C | C |
| 112 | B | C |
| 113 | B | B |
| 114 | B | D |
| 115 | C | C |
| 116 | A | B |
| 117 | D | B |
| 118 | D | C |
| 119 | D | B |
| 120 | C | B |
| 121 | B | B |
| 122 | C | C |
| 123 | B | B |
| 124 | C | B |
| 125 | C | C |
| 126 | C | C |
| 127 | C | B |
| 128 | C | C |
| 129 | C | C |
| 130 | C | C |
| 131 | C | C |
| 132 | A | B |
| 133 | B | C |
| 134 | B | C |
| 135 | B | B |
| 136 | C | B |
| 137 | D | D |
| 138 | D | D |
| 139 | D | D |
| 140 | C | B |
| 141 | A | B |
| 142 | B | B |
| 143 | A | B |
| 144 | C | C |
| 145 | D | D |
| 146 | C | C |
| 147 | B | C |
| 148 | B | B |
| 149 | C | C |
| 150 | C | B |
| 151 | D | D |
| 152 | D | D |
| 153 | C | C |
| 154 | D | D |
| 155 | B | B |
| 156 | C | C |
| 157 | B | C |
| 158 | B | B |
| 159 | B | B |
| 160 | A | B |
| 161 | A | B |
| 162 | C | C |
| 163 | A | C |
| 164 | C | C |
| 165 | C | C |
| 166 | D | C |
| 167 | C | B |
| 168 | D | C |
| 169 | B | B |
| 170 | B | B |
| 171 | C | C |
| 172 | D | D |
| 173 | C | C |
| 174 | C | C |
| 175 | C | C |
| 176 | D | D |
| 177 | A | A |
| 178 | C | C |
| 179 | B | D |
| 180 | C | C |
| 181 | B | C |
| 182 | D | A |
| 183 | C | C |
| 184 | C | B |
| 185 | C | C |
| 186 | C | D |
| 187 | C | C |
| 188 | C | C |
| 189 | D | D |
| 190 | B | D |
| 191 | B | D |
| 192 | B | C |
| 193 | D | D |
| 194 | C | D |
| 195 | C | C |
| 196 | D | C |
| 197 | C | D |
| 198 | D | D |
| 199 | D | D |
| 200 | D | D |
| 201 | A | D |
| 202 | D | D |
| 203 | A | C |
| 204 | D | D |
| 205 | B | B |
| 206 | B | D |
| 207 | D | D |
| 208 | C | D |
| 209 | C | D |
| 210 | C | D |
| 211 | B | D |
| 212 | D | D |
| 213 | B | C |
| 214 | D | D |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### PRIORITY

This application claims the benefit of priority to U.S. Provisional Application No. 63/126,433, filed Dec. 16, 2020, which application is hereby incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360474_402_SEQUENCE_LISTING.txt. The text file is 33 KB, was created on Dec. 16, 2021, and is being submitted electronically via EFS-Web.

1. A compound having structure (I): 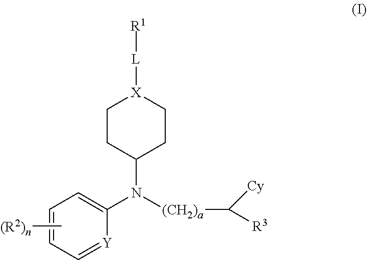
or a pharmaceutically acceptable salt, solvate, hydrate, isomer, or isotope thereof, wherein:
R¹ is an 8-13 membered heteroaryl comprising 1-4 ring nitrogen atoms and substituted with 1, 2, 3 or 4 substituents, wherein each substituent is independently OH, oxo, halo, CN, NO₂, C₁₋₄ alkyl, O—C₁₋₄ alkyl, C₁₋₄ alkenyl or C₃₋₆ cycloalkyl;
X is N or CH;
when X is N, then L is a bond, CH₂, C(O) or CHMe, and
when X is CH, then L is a bond, NH, NMe or NHC(O);
Y is N or CH;
each occurrence of R² is independently halo, OH or OMe;
a is 0, 1 or 2;
n is 0, 1, 2, 3 or 4;
R³ is H or C₁₋₄ alkyl; and
Cy is a 3-6 membered cycloalkyl or heterocycloalkyl comprising 0 or 1 ring oxygen atom.

2. The compound of claim 1, or a pharmaceutically acceptable salt, solvate, hydrate, isomer, or isotope thereof, wherein the compound has one of the following structures (I-A), (I-B), (I-B-cis) or (I-B-trans):

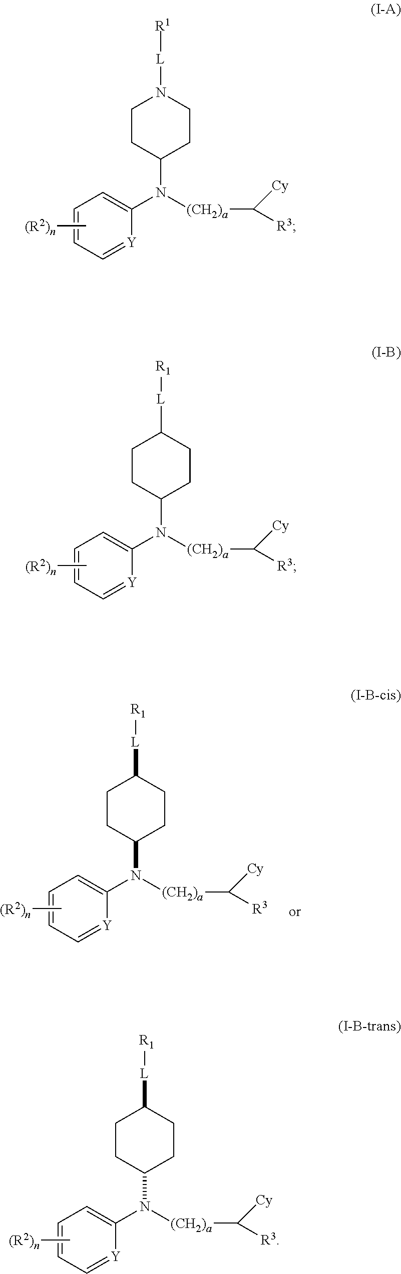

3. The compound of claim 1, or a pharmaceutically acceptable salt, solvate, hydrate, isomer, or isotope thereof, wherein the compound has one of the following structures (I-A-1), (I-A-2), (I-A-3), (I-A-4), (I-B-1), (I-B-2), (I-B-3) or (I-B-4):

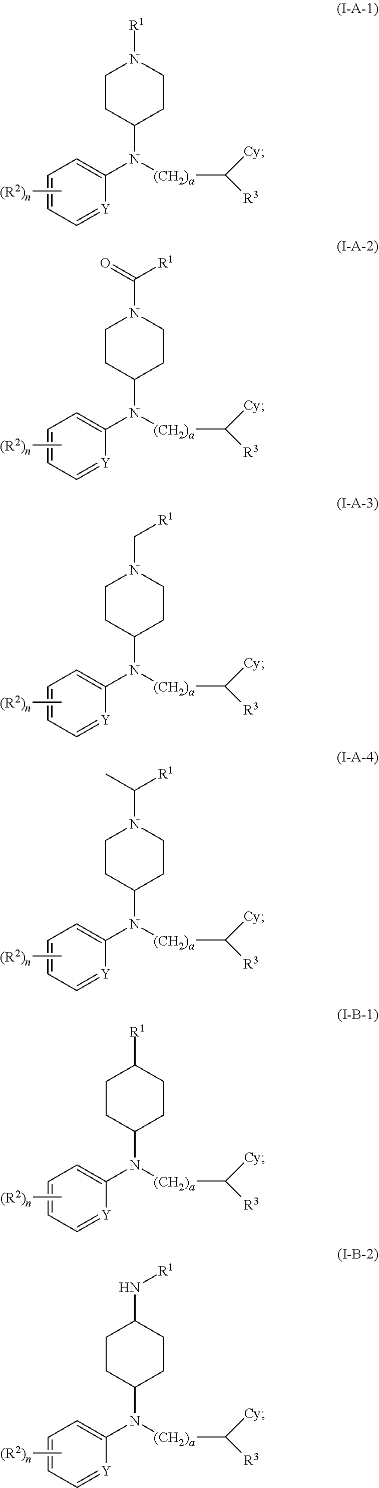

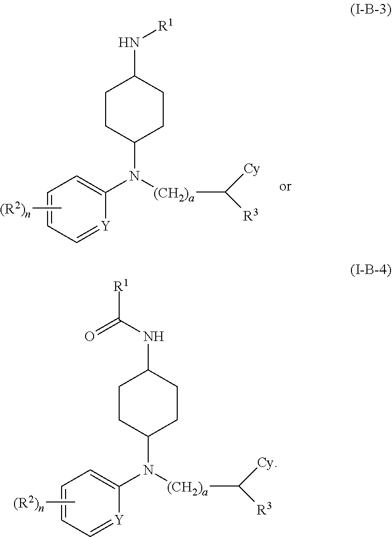

4-15. (canceled)

16. The compound of claim 1, or a pharmaceutically acceptable salt, solvate, hydrate, isomer, or isotope thereof, wherein R¹ is:

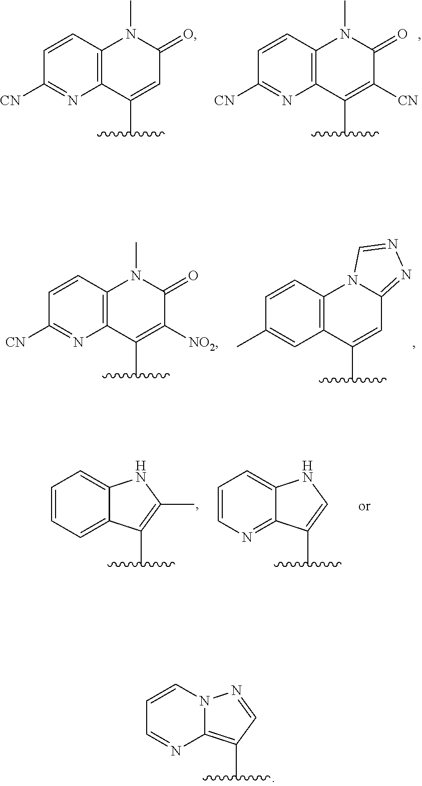

17-27. (canceled)

28. The compound of claim 1, or a pharmaceutically acceptable salt, solvate, hydrate, isomer, or isotope thereof wherein 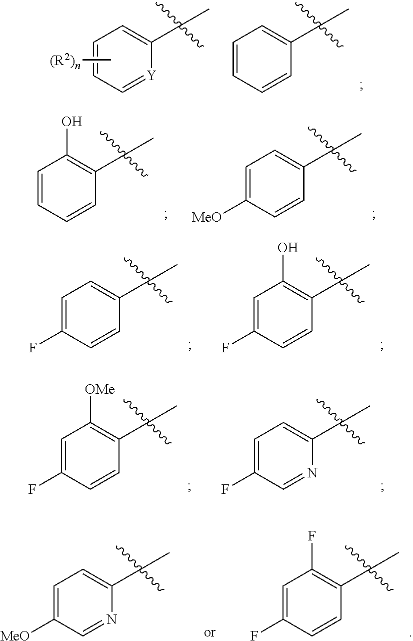
has one of the following structures:

29-38. (canceled)

39. A compound having the structure of any one of the following compounds listed in Table 1:

| CMPD. NO. | STRUCTURE | NAME |
|---|---|---|
| 1A | 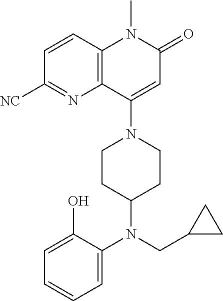 [SMILES: Cn1c(=O)cc(N2CCC(N(CC3CC3)c3ccccc3O)CC2)c2nc(C#N)ccc21] | 8-(4-((cyclopropylmethyl)(2-hydroxyphenyl)amino) piperidin-1-yl)-5-methyl-6-oxo-5,6-dihydro-1,5- naphthyridine-2-carbonitrile |
| 1B | 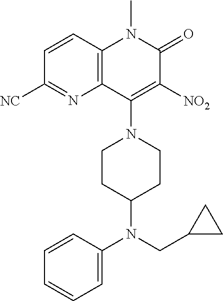 [SMILES: Cn1c(=O)c([N+](=O)[O-])c(N2CCC(N(CC3CC3)c3ccccc3)CC2)c2nc(C#N)ccc21] | 8-(4-((cyclopropylmethyl)(phenyl)amino)piperidin-1- yl)-5-methyl-7-nitro-6-oxo-5,6-dihydro-1,5- naphthyridine-2-carbonitrile |
| 1C | 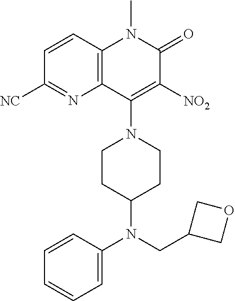 [SMILES: Cn1c(=O)c([N+](=O)[O-])c(N2CCC(N(CC3COC3)c3ccccc3)CC2)c2nc(C#N)ccc21] | 5-methyl-7-nitro-8-(4-((oxetan-3-ylmethyl)(phenyl) amino)piperidin-1-yl)-6-oxo-5,6-dihydro-1,5- naphthyridine-2-carbonitrile |
| 1D | 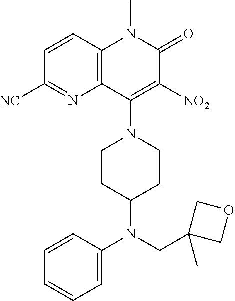 [SMILES: Cn1c(=O)c([N+](=O)[O-])c(N2CCC(N(CC3(C)COC3)c3ccccc3)CC2)c2nc(C#N)ccc21] | 5-methyl-8-(4-(((3-methyloxetan-3-yl)methyl)(phenyl) amino)piperidin-1-yl)-7-nitro-6-oxo-5,6-dihydro-1,5- naphthyridine-2-carbonitrile |
| 1E | 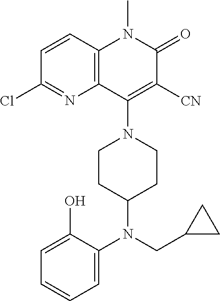 [SMILES: Cn1c(=O)c(C#N)c(N2CCC(N(CC3CC3)c3ccccc3O)CC2)c2nc(Cl)ccc21] | 6-chloro-4-(4-((cyclopropylmethyl)(2-hydroxyphenyl) amino)piperidin-1-yl)-1-methyl-2-oxo-1,2-dihydro-1,5- naphthyridine-3-carbonitrile |
| 1F | 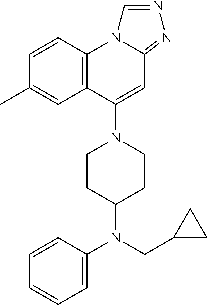 [SMILES: Cc1ccc2c(c1)c(N1CCC(N(CC3CC3)c3ccccc3)CC1)cc1nncn12] | N-(cyclopropylmethyl)-1-(7-methyl-[1,2,4] triazolo[4,3-a]quinolin-5-yl)-N-phenylpiperidin-4- amine |
| 2A | 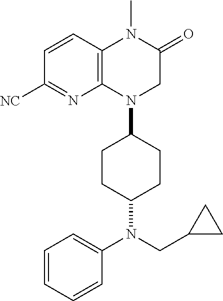 | trans-4-[4-[N-(cyclopropylmethyl)anilino]cyclohexyl]- 1-methyl-2-oxo-3H-pyrido[2,3-b]pyrazine-6- carbonitrile |
| 2B | 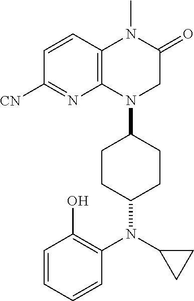 | 4-(trans-4-(cyclopropyl(2-hydroxyphenyl)amino) cyclohexyl)-1-methyl-2-oxo-1,2,3,4- tetrahydropyrido[3,2-b]pyrazine-6-carbonitrile |
| 2C | 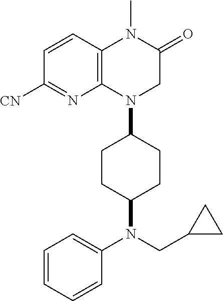 | cis-4-[4-[N-(cyclopropylmethyl)anilino]cyclohexyl]-1- methyl-2-oxo-3H-pyrido[2,3-b]pyrazine-6-carbonitrile |
| 3A | 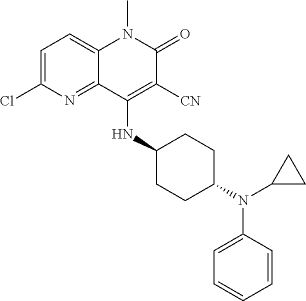 [SMILES: Cn1c(=O)c(C#N)c(N[C@H]2CC[C@H](N(c3ccccc3)C3CC3)CC2)c2nc(Cl)ccc21] | trans-6-chloro-4-((4-(cyclopropyl(phenyl)amino) cyclohexyl)amino)-1-methyl-2-oxo-1,2-dihydro-1,5- naphthyridine-3-carbonitrile |
| 3B | 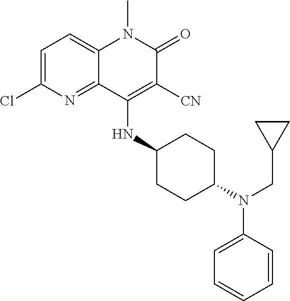 [SMILES: Cn1c(=O)c(C#N)c(N[C@H]2CC[C@H](N(CC3CC3)c3ccccc3)CC2)c2nc(Cl)ccc21] | trans-6-chloro-4-((4-((cyclopropylmethyl)(phenyl) amino)cyclohexyl)amino)-1-methyl-2-oxo-1,2- dihydro-1,5-naphthyridine-3-carbonitrile |
| 3C | 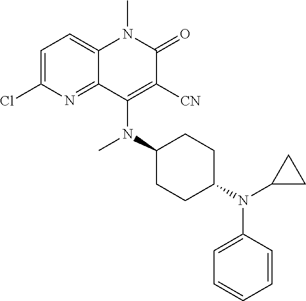 [SMILES: Cn1c(=O)c(C#N)c(N(C)[C@H]2CC[C@H](N(c3ccccc3)C3CC3)CC2)c2nc(Cl)ccc21] | trans-6-chloro-4-((4-(cyclopropyl(phenyl)amino) cyclohexyl)(methyl)amino)-1-methyl-2-oxo-1,2- dihydro-1,5-naphthyridine-3-carbonitrile |
| 3D | 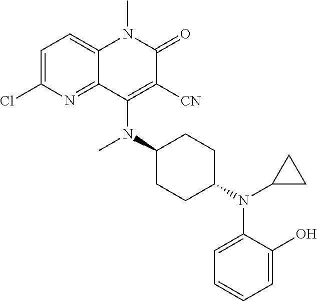 [SMILES: Cn1c(=O)c(C#N)c(N(C)[C@H]2CC[C@H](N(c3ccccc3O)C3CC3)CC2)c2nc(Cl)ccc21] | trans-6-chloro-4-((4-(cyclopropyl(2-hydroxyphenyl) amino)cyclohexyl)(methyl)amino)-1-methyl-2-oxo- 1,2-dihydro-1,5-naphthyridine-3-carbonitrile |
| 3E | 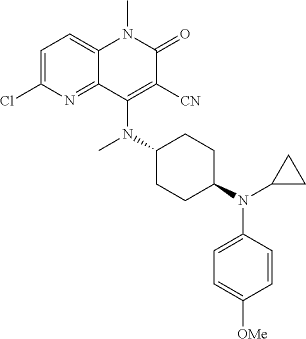 [SMILES: COc1ccc(N(C2CC2)[C@H]2CC[C@H](N(C)c3c(C#N)c(=O)n(C)c4ccc(Cl)nc34)CC2)cc1] | trans-6-chloro-4-((4-(cyclopropyl(4-methoxyphenyl) amino)cyclohexyl)(methyl)amino)-1-methyl-2-oxo- 1,2-dihydro-1,5-naphthyridine-3-carbonitrile |
| 3F | 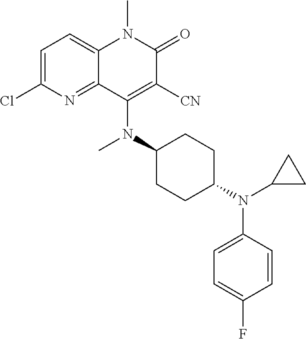 [SMILES: Cn1c(=O)c(C#N)c(N(C)[C@H]2CC[C@H](N(c3ccc(F)cc3)C3CC3)CC2)c2nc(Cl)ccc21] | trans-6-chloro-4-((4-(cyclopropyl(4-fluorophenyl) amino)cyclohexyl)(methyl)amino)-1-methyl-2-oxo- 1,2-dihydro-1,5-naphthyridine-3-carbonitrile |
| 3G | 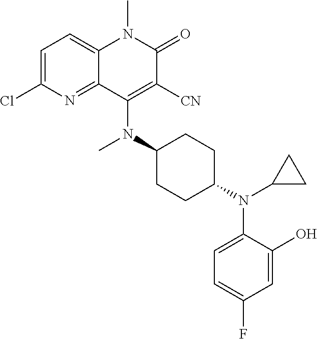 [SMILES: Cn1c(=O)c(C#N)c(N(C)[C@H]2CC[C@H](N(c3ccc(F)cc3O)C3CC3)CC2)c2nc(Cl)ccc21] | trans-6-chloro-4-((4-(cyclopropyl(4-fluoro-2- hydroxyphenyl)amino)cyclohexyl)(methyl)amino)-1- methyl-2-oxo-1,2-dihydro-1,5-naphthyridine-3- carbonitrile |
| 3H | 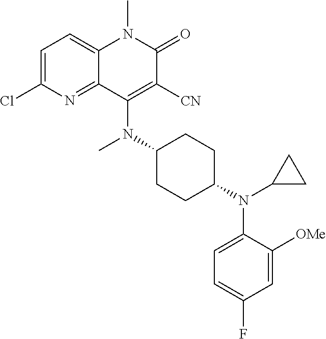 [SMILES: COc1cc(F)ccc1N(C1CC1)[C@H]1CC[C@@H](N(C)c2c(C#N)c(=O)n(C)c3ccc(Cl)nc23)CC1] | cis-6-chloro-4-((4-(cyclopropyl(4-fluoro-2- methoxyphenyl)amino)cyclohexyl)(methyl)amino)-1- methyl-2-oxo-1,2-dihydro-1,5-naphthyridine-3- carbonitrile |
| 3I | 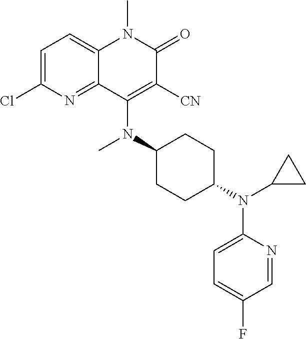 [SMILES: Cn1c(=O)c(C#N)c(N(C)[C@H]2CC[C@H](N(c3ccc(F)cn3)C3CC3)CC2)c2nc(Cl)ccc21] | trans-6-chloro-4-[[4-[cyclopropyl-(5-fluoro-2-pyridyl) amino]cyclohexyl]-methyl-amino]-1-methyl-2-oxo- 1,5-naphthyridine-3-carbonitrile |
| 3J | 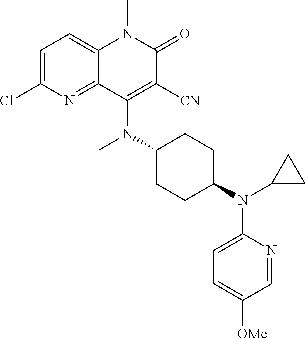 [SMILES: COc1ccc(N(C2CC2)[C@H]2CC[C@H](N(C)c3c(C#N)c(=O)n(C)c4ccc(Cl)nc34)CC2)nc1] | trans-6-chloro-4-((4-(cyclopropyl(5-methoxypyridin-2- yl)amino)cyclohexyl)(methyl)amino)-1-methyl-2-oxo- 1,2-dihydro-1,5-naphthyridine-3-carbonitrile |
| 3K | 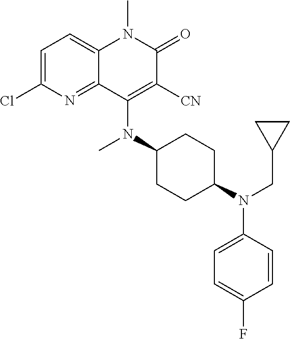 [SMILES: Cn1c(=O)c(C#N)c(N(C)[C@H]2CC[C@@H](N(CC3CC3)c3ccc(F)cc3)CC2)c2nc(Cl)ccc21] | cis-6-Chloro-4-((4-((cyclopropylmethyl)(4- fluorophenyl)amino)cyclohexyl)amino)-1-methyl-2- oxo-1,2-dihydro-1,5-naphthyridine-3-carbonitrile |
| 3L | 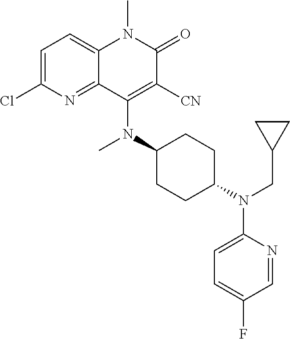 [SMILES: Cn1c(=O)c(C#N)c(N(C)[C@H]2CC[C@H](N(CC3CC3)c3ccc(F)cn3)CC2)c2nc(Cl)ccc21] | trans-6-chloro-4-((4-((cyclopropylmethyl)(5- fluoropyridin-2-yl)amino)cyclohexyl)(methyl)amino)- 1-methyl-2-oxo-1,2-dihydro-1,5-naphthyridine-3- carbonitrile |
| 3M | 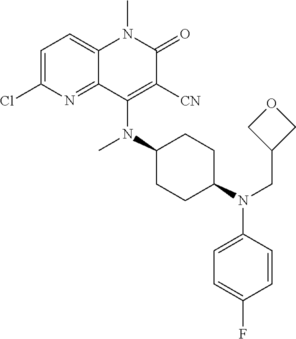 [SMILES: Cn1c(=O)c(C#N)c(N(C)[C@H]2CC[C@@H](N(CC3COC3)c3ccc(F)cc3)CC2)c2nc(Cl)ccc21] | cis-6-Chloro-4-[[4-(N-4-fluorophenyl-N-oxetan-3- ylmethylanilino)cyclohexyl]-methylamino]-1-methyl- 2-oxo-1,2-dihydro-1,5-naphthyridine-3-carbonitrile |
| 3N | 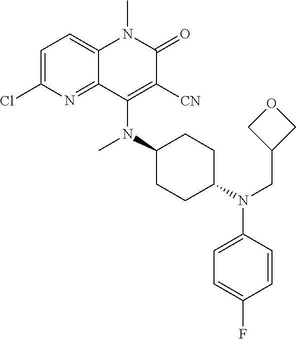 [SMILES: Cn1c(=O)c(C#N)c(N(C)[C@H]2CC[C@H](N(CC3COC3)c3ccc(F)cc3)CC2)c2nc(Cl)ccc21] | trans-6-Chloro-4-[[4-(N-4-fluorophenyl-N-oxetan-3- ylmethylanilino)cyclohexyl]-methylamino]-1-methyl- 2-oxo-1,2-dihydro-1,5-naphthyridine-3-carbonitrile |
| 3O | 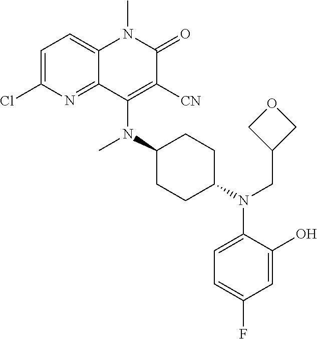 [SMILES: Cn1c(=O)c(C#N)c(N(C)[C@H]2CC[C@H](N(CC3COC3)c3ccc(F)cc3O)CC2)c2nc(Cl)ccc21] | trans-6-chloro-4-((-4-((4-fluoro-2-hydroxyphenyl) (oxetan-3-ylmethyl)amino)cyclohexyl)(methyl)amino)- 1-methyl-2-oxo-1,2-dihydro-1,5-naphthyridine-3- carbonitrile |
| 3Q | 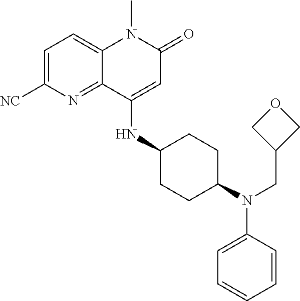 [SMILES: Cn1c(=O)cc(N[C@H]2CC[C@@H](N(CC3COC3)c3ccccc3)CC2)c2nc(C#N)ccc21] | cis-5-methyl-8-((4-((oxetan-3-ylmethyl)(phenyl)amino) cyclohexyl)amino)-6-oxo-5,6-dihydro-1,5- naphthyridine-2-carbonitrile |
| 3R | 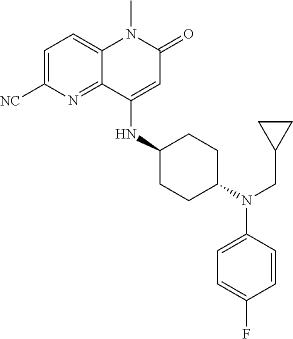 [SMILES: Cn1c(=O)cc(N[C@H]2CC[C@H](N(CC3CC3)c3ccc(F)cc3)CC2)c2nc(C#N)ccc21] | trans-8-((4-((cyclopropylmethyl)(4-fluorophenyl) amino)cyclohexyl)amino)-5-methyl-6-oxo-5,6- dihydro-1,5-naphthyridine-2-carbonitrile |
| 3S | 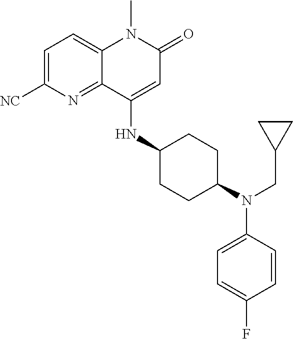 [SMILES: Cn1c(=O)cc(N[C@H]2CC[C@@H](N(CC3CC3)c3ccc(F)cc3)CC2)c2nc(C#N)ccc21] | cis-8-((4-((cyclopropylmethyl)(4-fluorophenyl)amino) cyclohexyl)amino)-5-methyl-6-oxo-5,6-dihydro-1,5- naphthyridine-2-carbonitrile |
| 3T | 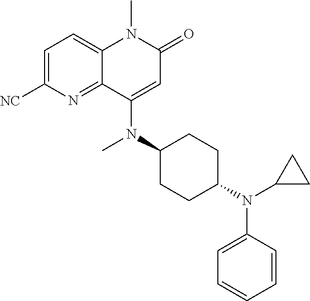 [SMILES: Cn1c(=O)cc(N(C)[C@H]2CC[C@H](N(c3ccccc3)C3CC3)CC2)c2nc(C#N)ccc21] | trans-8-((4-(cyclopropyl(phenyl)amino)cyclohexyl) (methyl)amino)-5-methyl-6-oxo-5,6-dihydro-1,5- naphthyridine-2-carbonitrile |
| 3U | 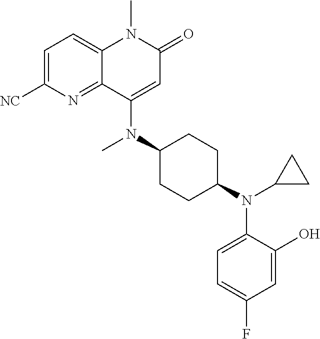 [SMILES: Cn1c(=O)cc(N(C)[C@H]2CC[C@@H](N(c3ccc(F)cc3O)C3CC3)CC2)c2nc(C#N)ccc21] | cis-8-[[4-(N-cyclopropyl-4-fluoro-2-methoxy-anilino) cyclohexyl]-methyl-amino]-5-methyl-6-oxo-1,5- naphthyridine-2-carbonitrile |
| 3V | 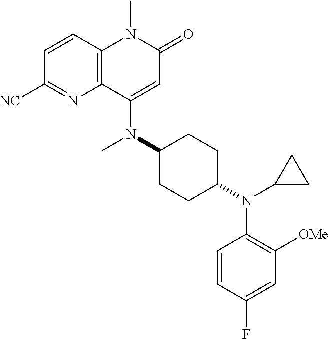 | trans-8-[[4-(N-cyclopropyl-4-fluoro-2-methoxy- anilino)cyclohexyl]-methyl-amino]-5-methyl-6-oxo- 1,5-naphthyridine-2-carbonitrile |
| 3W | 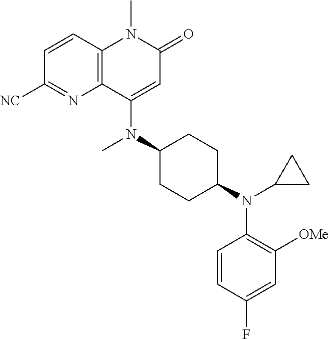 [SMILES: COc1cc(F)ccc1N(C1CC1)[C@H]1CC[C@@H](N(C)c2cc(=O)n(C)c3ccc(C#N)nc23)CC1] | cis-8-[[4-(N-cyclopropyl-4-fluoro-2-methoxy-anilino) cyclohexyl]-methyl-amino]-5-methyl-6-oxo-1,5- naphthyridine-2-carbonitrile |
| 3X | 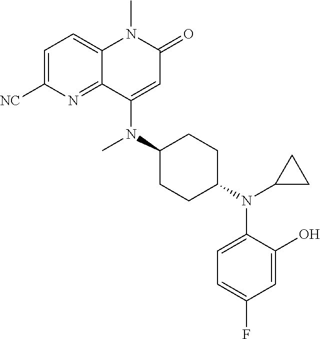 [SMILES: Cn1c(=O)cc(N(C)[C@H]2CC[C@H](N(c3ccc(F)cc3O)C3CC3)CC2)c2nc(C#N)ccc21] | trans-8-((4-(cyclopropyl(4-fluoro-2-hydroxyphenyl) amino)cyclohexyl)(methyl)amino)-5-methyl-6-oxo- 5,6-dihydro-1,5-naphthyridine-2-carbonitrile |
| 3Y | 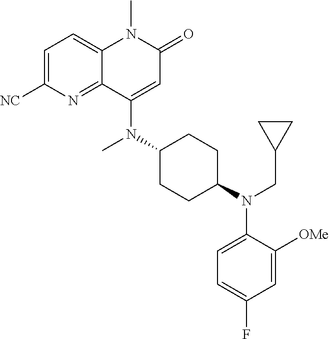 [SMILES: COc1cc(F)ccc1N(CC1CC1)[C@H]1CC[C@H](N(C)c2cc(=O)n(C)c3ccc(C#N)nc23)CC1] | trans-8-[[4-(N-cyclopropylmethyl-N-4-fluoro-2- methoxyphenyl-anilino)cyclohexyl]-methyl-amino]-5- methyl-6-oxo-5,6-dihydro-1,5-naphthyridine-2- carbonitrile |
| 3Z | 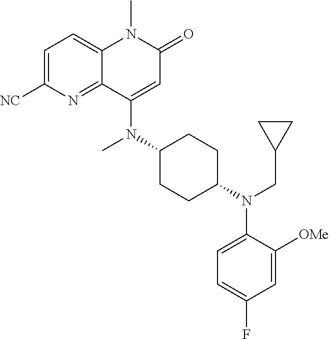 [SMILES: COc1cc(F)ccc1N(CC1CC1)[C@H]1CC[C@@H](N(C)c2cc(=O)n(C)c3ccc(C#N)nc23)CC1] | cis-8-[[4-(N-cyclopropylmethyl-N-4-fluoro-2- methoxyphenyl-anilino)cyclohexyl]-methyl-amino]-5- methyl-6-oxo-5,6-dihydro-1,5-naphthyridine-2- carbonitrile |
| 3AA | 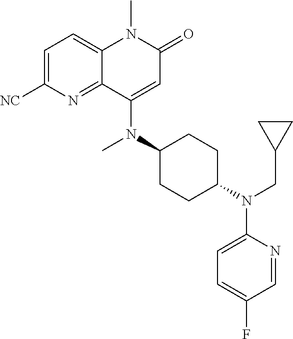 [SMILES: Cn1c(=O)cc(N(C)[C@H]2CC[C@H](N(CC3CC3)c3ccc(F)cn3)CC2)c2nc(C#N)ccc21] | trans-8-[[4-[cyclopropylmethyl-(5-fluoro-2-pyridyl) amino]cyclohexyl]-methyl-amino]-5-methyl-6-oxo- 1,5-naphthyridine-2-carbonitrile |
| 3BB | 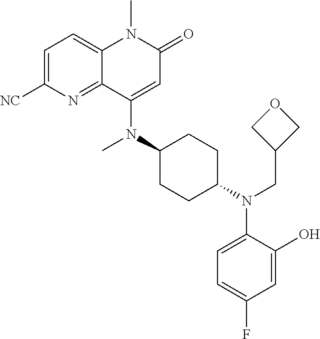 [SMILES: Cn1c(=O)cc(N(C)[C@H]2CC[C@H](N(CC3COC3)c3ccc(F)cc3O)CC2)c2nc(C#N)ccc21] | trans-8-((4-((4-fluoro-2-hydroxyphenyl)(oxetan-3- ylmethyl)amino)cyclohexyl)(methyl)amino)-5-methyl- 6-oxo-5,6-dihydro-1,5-naphthyridine-2-carbonitrile |
| 3CC | 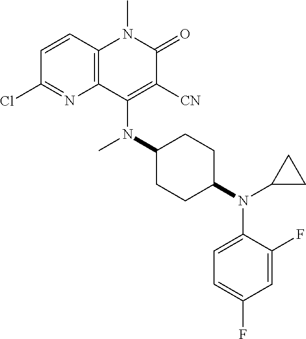 | cis-6-Chloro-4-[[4-(N-cyclopropyl-2,4-difluoro- anilino)cyclohexyl]-methyl-amino]-1-methyl-2-oxo- 1,5-naphthyridine-3-carbonitrile |
| 4A | 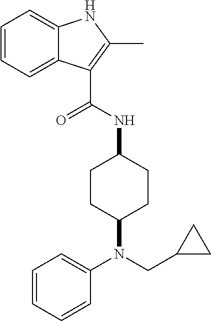 | cis-N-4-((cyclopropylmethyl)(phenyl)amino) cyclohexyl)-2-methyl-1H-indole-3-carboxamide |
| 4B | 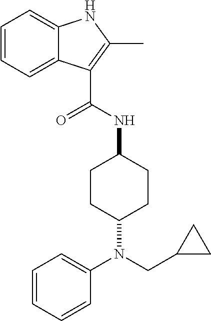 | trans-N-4-((cyclopropylmethyl)(phenyl)amino) cyclohexyl)-2-methyl-1H-indole-3-carboxamide |
| 4C | 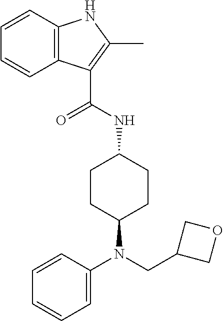 [SMILES: Cc1[nH]c2ccccc2c1C(=O)N[C@H]1CC[C@H](N(CC2COC2)c2ccccc2)CC1] | trans-2-methyl-N-(4-((oxetan-3-ylmethyl)(phenyl) amino)cyclohexyl)-1H-indole-3-carboxamide |
| 4D | 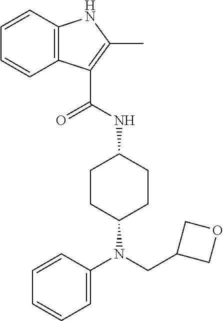 [SMILES: Cc1[nH]c2ccccc2c1C(=O)N[C@H]1CC[C@@H](N(CC2COC2)c2ccccc2)CC1] | cis-2-methyl-N-(4-((oxetan-3-ylmethyl)(phenyl)amino) cyclohexyl)-1H-indole-3-carboxamide |
| 4E | 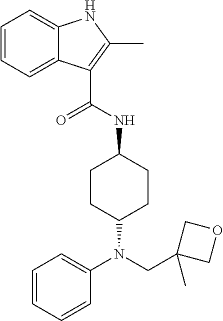 | trans-2-methyl-N-(4-(((3-methyloxetan-3-yl)methyl) (phenyl)amino)cyclohexyl)-1H-indole-3-carboxamide |
| 4F | 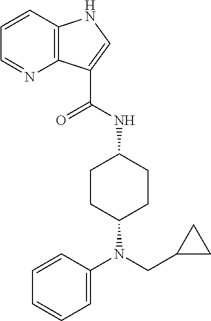 [SMILES: O=C(N[C@H]1CC[C@@H](N(CC2CC2)c2ccccc2)CC1)c1c[nH]c2cccnc12] | cis-N-(4-((cyclopropylmethyl)(phenyl)amino) cyclohexyl)-1H-pyrrolo[3,2-b]pyridine-3-carboxamide |
| 4G | 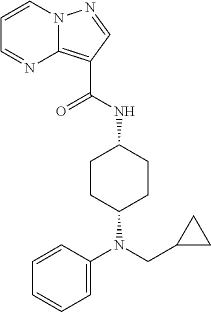 [SMILES: O=C(N[C@H]1CC[C@@H](N(CC2CC2)c2ccccc2)CC1)c1cnn2cccnc12] | cis-N-(4-((cyclopropylmethyl)(phenyl)amino) cyclohexyl)pyrazolo[1,5-a]pyrimidine-3-carboxamide |
| 5 | 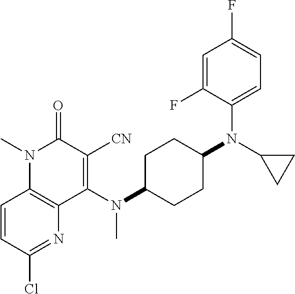 | cis-6-chloro-4-((-4-(cyclopropyl(2,4-difluorophenyl) amino)cyclohexyl)(methyl)amino)-1-methyl-2-oxo- 1,2-dihydro-1,5-naphthyridine-3-carbonitrile |
| 6 | 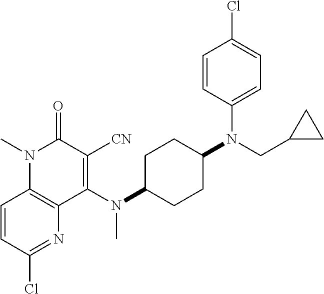 | cis-6-chloro-4-((4-((4-chlorophenyl) (cyclopropylmethyl)amino)cyclohexyl)(methyl) amino)-1-methyl-2-oxo-1,2-dihydro-1,5- naphthyridine-3-carbonitrile |
| 7 | 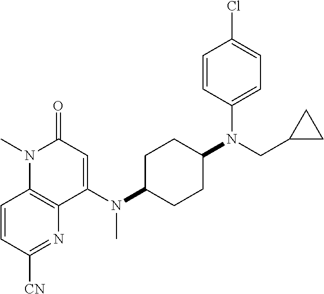 | cis-8-((4-((4-chlorophenyl)(cyclopropylmethyl) amino)cyclohexyl)(methyl)amino)-5-methyl-6-oxo- 5,6-dihydro-1,5-naphthyridine-2-carbonitrile |
| 8 | 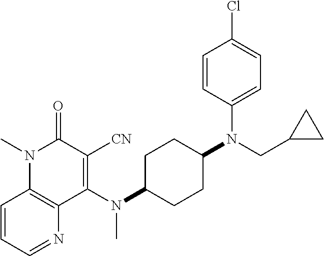 | cis-4-((4-((4-chlorophenyl)(cyclopropylmethyl) amino)cyclohexyl)(methyl)amino)-1-methyl-2-oxo- 1,2-dihydro-1,5-naphthyridine-3-carbonitrile |
| 9 | 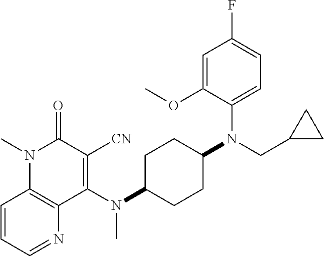 | cis-4-((4-((cyclopropylmethyl)(4-fluoro-2- methoxyphenyl)amino)cyclohexyl)(methyl)amino)-1- methyl-2-oxo-1,2-dihydro-1,5-naphthyridine-3- carbonitrile |
| 10 | 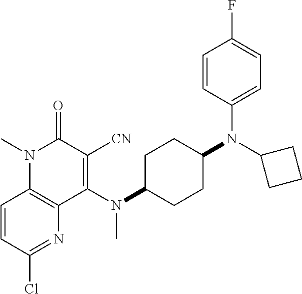 | cis-6-chloro-4-((4-(cyclobutyl(4-fluorophenyl)amino) cyclohexyl)(methyl)amino)-1-methyl-2-oxo-1,2- dihydro-1,5-naphthyridine-3-carbonitrile |
| 11 | 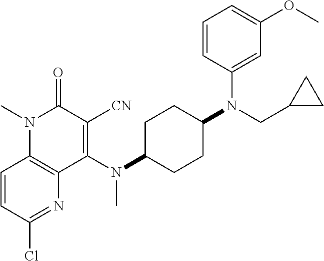 | cis-6-chloro-4-((4-((cyclopropylmethyl)(3- methoxyphenyl)amino)cyclohexyl)(methyl)amino)-1- methyl-2-oxo-1,2-dihydro-1,5-naphthyridine-3- carbonitrile |
| 12 | 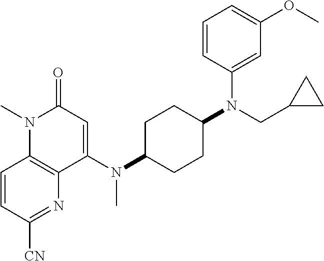 | cis-8-((4-((cyclopropylmethyl)(3-methoxyphenyl) amino)cyclohexyl)(methyl)amino)-5-methyl-6-oxo- 5,6-dihydro-1,5-naphthyridine-2-carbonitrile |
| 13 | 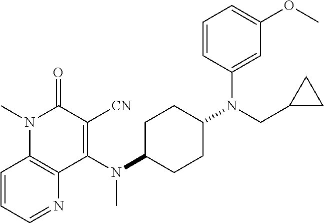 | trans-4-((4-((cyclopropylmethyl)(3-methoxyphenyl) amino)cyclohexyl)(methyl)amino)-1-methyl-2-oxo- 1,2-dihydro-1,5-naphthyridine-3-carbonitrile |
| 14 | 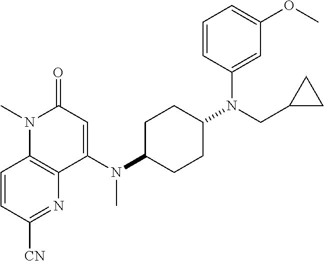 | trans-8-((4-((cyclopropylmethyl)(3-methoxyphenyl) amino)cyclohexyl)(methyl)amino)-5-methyl-6-oxo- 5,6-dihydro-1,5-naphthyridine-2-carbonitrile |
| 15 | 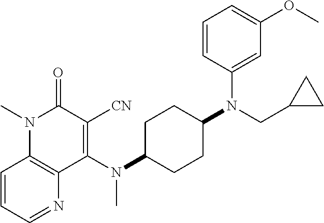 | cis-4-((4-((cyclopropylmethyl)(3-methoxyphenyl) amino)cyclohexyl)(methyl)amino)-1-methyl-2-oxo- 1,2-dihydro-1,5-naphthyridine-3-carbonitrile |
| 16 | 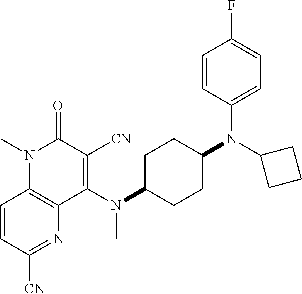 | cis-6-chloro-4-((4-(cyclobutyl(4-fluorophenyl)amino) cyclohexyl)(methyl)amino)-1-methyl-2-oxo-1,2- dihydro-1,5-naphthyridine-3-carbonitrile |
| 17 | 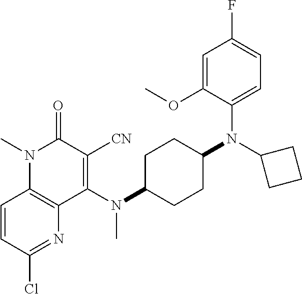 | cis-6-chloro-4-((4-(cyclobutyl(4-fluoro-2- methoxyphenyl)amino)cyclohexyl)(methyl)amino)-1- methyl-2-oxo-1,2-dihydro-1,5-naphthyridine-3- carbonitrile |
| 18 | 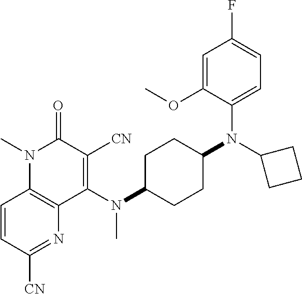 | cis-8-((4-(cyclobutyl(4-fluoro-2-methoxyphenyl) amino)cyclohexyl)(methyl)amino)-5-methyl-6-oxo- 5,6-dihydro-1,5-naphthyridine-2,7-dicarbonitrile |
| 19 | 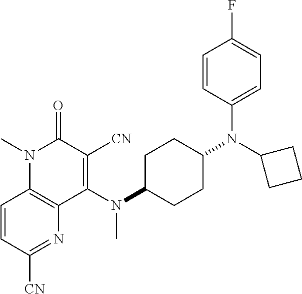 | trans-8-((4-(cyclobutyl(4-fluorophenyl)amino) cyclohexyl)(methyl)amino)-5-methyl-6-oxo-5,6- dihydro-1,5-naphthyridine-2,7-dicarbonitrile |
| 20 | 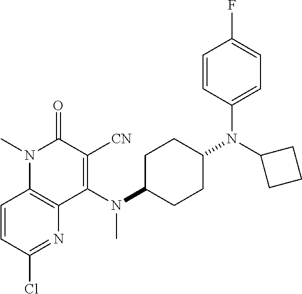 | trans-6-chloro-4-((4-(cyclobutyl(4-fluorophenyl) amino)cyclohexyl)(methyl)amino)-1-methyl-2-oxo- 1,2-dihydro-1,5-naphthyridine-3-carbonitrile |
| 21 | 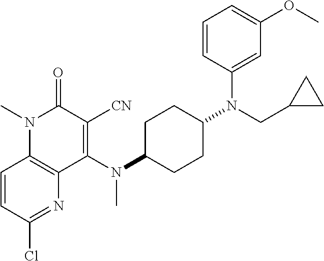 | trans-6-chloro-4-((4-((cyclopropylmethyl)(3- methoxyphenyl)amino)cyclohexyl)(methyl)amino)-1- methyl-2-oxo-1,2-dihydro-1,5-naphthyridine-3- carbonitrile |
| 22 | 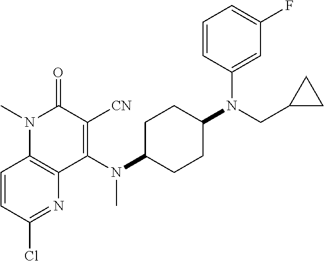 | cis-6-chloro-4-((4-((cyclopropylmethyl)(3- fluorophenyl)amino)cyclohexyl)(methyl)amino)-1- methyl-2-oxo-1,2-dihydro-1,5-naphthyridine-3- carbonitrile |
| 23 | 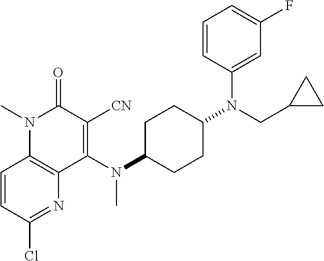 | trans-6-chloro-4-((4-((cyclopropylmethyl)(3- fluorophenyl)amino)cyclohexyl)(methyl)amino)-1- methyl-2-oxo-1,2-dihydro-1,5-naphthyridine-3- carbonitrile |
| 24 | 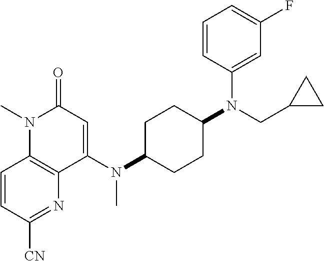 | cis-8-((4-((cyclopropylmethyl)(3-fluorophenyl) amino)cyclohexyl)(methyl)amino)-5-methyl-6-oxo- 5,6-dihydro-1,5-naphthyridine-2-carbonitrile |
| 25 | 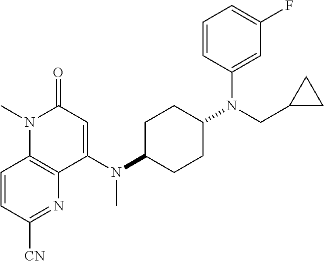 | trans-8-((4-((cyclopropylmethyl)(3-fluorophenyl) amino)cyclohexyl)(methyl)amino)-5-methyl-6-oxo- 5,6-dihydro-1,5-naphthyridine-2-carbonitrile |
| 26 | 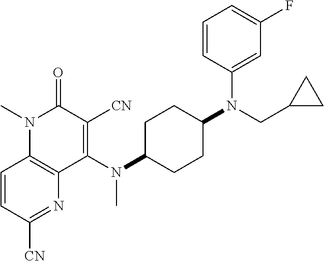 | cis-8-((4-((cyclopropylmethyl)(3-fluorophenyl) amino)cyclohexyl)(methyl)amino)-5-methyl-6-oxo- 5,6-dihydro-1,5-naphthyridine-2,7-dicarbonitrile |
| 27 | 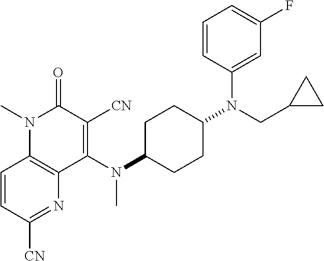 | trans-8-((4-((cyclopropylmethyl)(3-fluorophenyl) amino)cyclohexyl)(methyl)amino)-5-methyl-6-oxo- 5,6-dihydro-1,5-naphthyridine-2,7-dicarbonitrile |
| 28 | 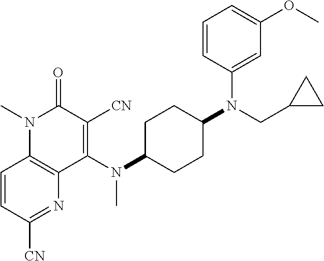 | cis-8-((4-((cyclopropylmethyl)(3-methoxyphenyl) amino)cyclohexyl)(methyl)amino)-5-methyl-6-oxo- 5,6-dihydro-1,5-naphthyridine-2,7-dicarbonitrile |
| 29 | 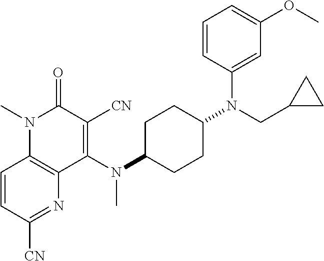 | trans-8-((4-((cyclopropylmethyl)(3-methoxyphenyl) amino)cyclohexyl)(methyl)amino)-5-methyl-6-oxo- 5,6-dihydro-1,5-naphthyridine-2,7-dicarbonitrile |
| 30 | 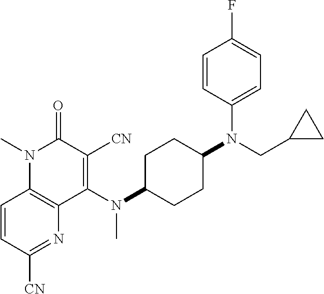 | cis-8-((4-((cyclopropylmethyl)(4-fluorophenyl) amino)cyclohexyl)(methyl)amino)-5-methyl-6-oxo- 5,6-dihydro-1,5-naphthyridine-2,7-dicarbonitrile |
| 31 | 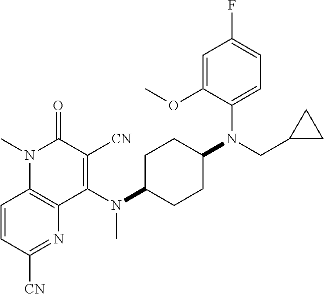 | cis-8-((4-((cyclopropylmethyl)(4-fluoro-2- methoxyphenyl)amino)cyclohexyl)(methyl)amino)-5- methyl-6-oxo-5,6-dihydro-1,5-naphthyridine-2,7- dicarbonitrile |
| 32 | 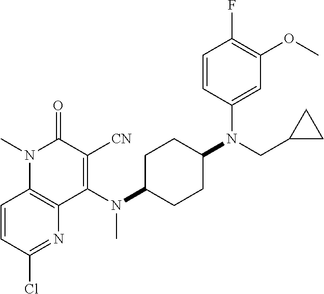 | cis-6-chloro-4-((4-((cyclopropylmethyl)(4-fluoro-3- methoxyphenyl)amino)cyclohexyl)(methyl)amino)-1- methyl-2-oxo-1,2-dihydro-1,5-naphthyridine-3- carbonitrile |
| 33 | 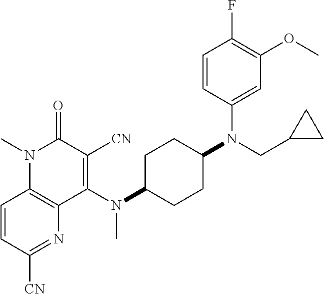 | cis-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2,7- dicarbonitrile |
| 34 | 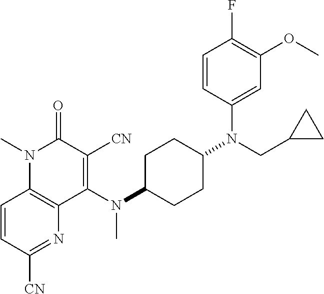 | trans-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2,7- dicarbonitrile |
| 35 | 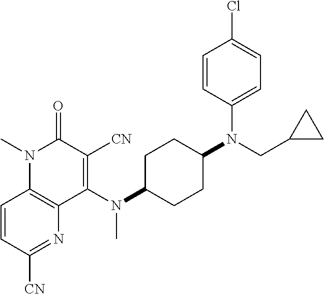 | cis-8-((4-((4-chlorophenyl)(cyclopropylmethyl) amino)cyclohexyl)(methyl)amino)-5-methyl-6-oxo- 5,6-dihydro-1,5-naphthyridine-2,7-dicarbonitrile |
| 36 | 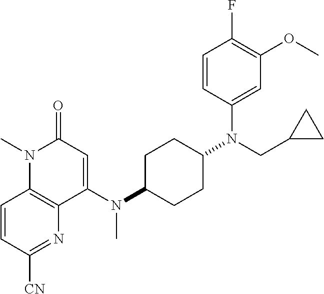 | trans-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2- carbonitrile |
| 37 | 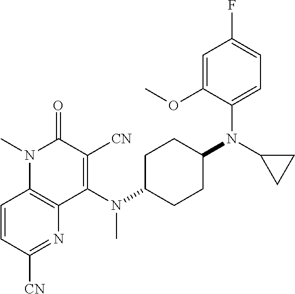 | trans-8-((4-(cyclopropyl(4-fluoro-2-methoxyphenyl) amino)cyclohexyl)(methyl)amino)-5-methyl-6-oxo- 5,6-dihydro-1,5-naphthyridine-2,7-dicarbonitrile |
| 38 | 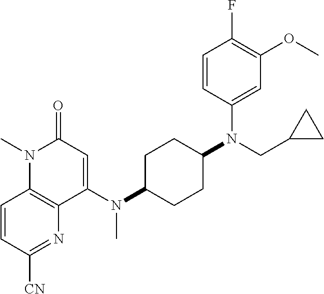 | cis-8-((4-((cyclopropylmethyl)(4-fluoro-3- methoxyphenyl)amino)cyclohexyl)(methyl)amino)-5- methyl-6-oxo-5,6-dihydro-1,5-naphthyridine-2- carbonitrile |
| 39 | 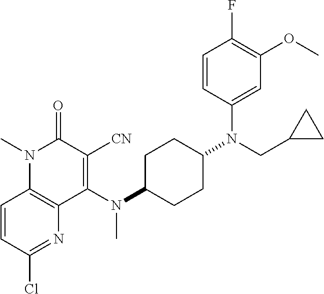 | trans-6-chloro-4-((4-((cyclopropylmethyl)(4-fluoro- 3-methoxyphenyl)amino)cyclohexyl)(methyl)amino)- 1-methyl-2-oxo-1,2-dihydro-1,5-naphthyridine-3- carbonitrile |
| 40 | 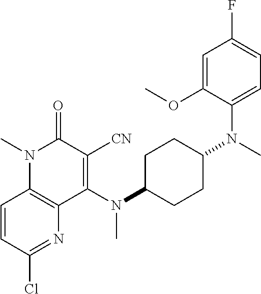 | trans-6-chloro-4-((4-((4-fluoro-2-methoxyphenyl) (methyl)amino)cyclohexyl)(methyl)amino)-1-methyl- 2-oxo-1,2-dihydro-1,5-naphthyridine-3-carbonitrile |
| 41 | 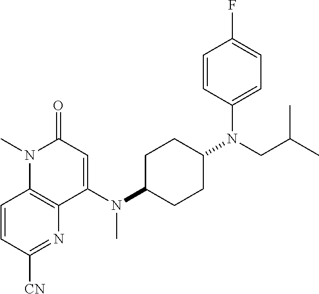 | trans-8-((-4-((4-fluorophenyl)(isobutyl)amino) cyclohexyl)(methyl)amino)-5-methyl-6-oxo-5,6- dihydro-1,5-naphthyridine-2-carbonitrile |
| 42 | 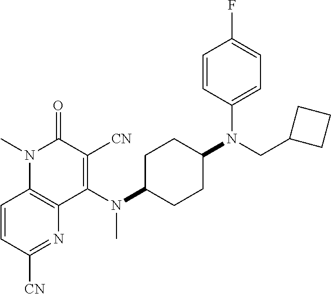 | cis-8-((-4-((cyclobutylmethyl)(4-fluorophenyl)amino) cyclohexyl)(methyl)amino)-5-methyl-6-oxo-5,6- dihydro-1,5-naphthyridine-2,7-dicarbonitrile |
| 43 | 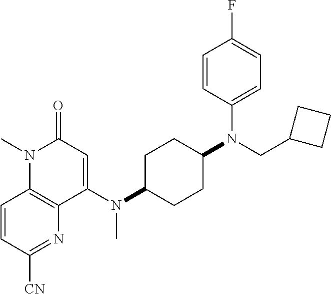 | cis-8-((-4-((cyclobutylmethyl)(4-fluorophenyl)amino) cyclohexyl)(methyl)amino)-5-methyl-6-oxo-5,6- dihydro-1,5-naphthyridine-2-carbonitrile |
| 44 | 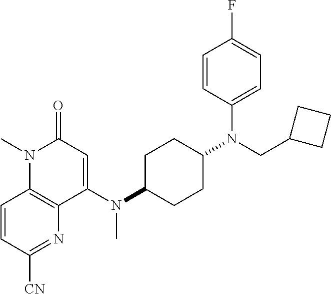 | trans-8-((-4-((cyclobutylmethyl)(4-fluorophenyl) amino)cyclohexyl)(methyl)amino)-5-methyl-6-oxo- 5,6-dihydro-1,5-naphthyridine-2-carbonitrile |
| 45 | 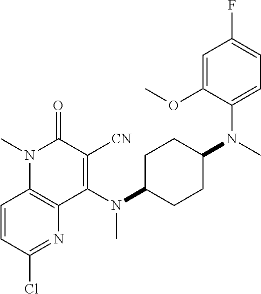 | cis-6-chloro-4-((4-((4-fluoro-2-methoxyphenyl) (methyl)amino)cyclohexyl)(methyl)amino)-1-methyl- 2-oxo-1,2-dihydro-1,5-naphthyridine-3-carbonitrile |
| 46 | 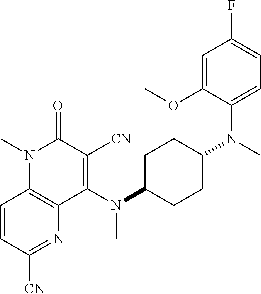 | trans-8-((4-((4-fluoro-2-methoxyphenyl)(methyl) amino)cyclohexyl)(methyl)amino)-5-methyl-6-oxo- 5,6-dihydro-1,5-naphthyridine-2,7-dicarbonitrile |
| 47 | 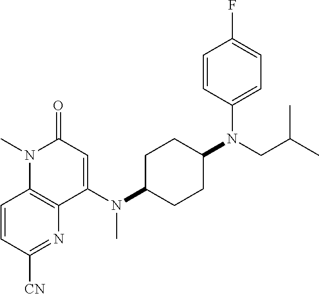 | cis-8-((-4-((4-fluorophenyl)(isobutyl)amino) cyclohexyl)(methyl)amino)-5-methyl-6-oxo-5,6- dihydro-1,5-naphthyridine-2-carbonitrile |
| 48 | 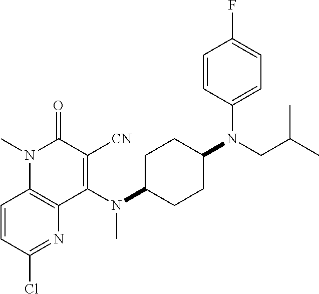 | cis-6-chloro-4-((-4-((4-fluorophenyl)(isobutyl)amino) cyclohexyl)(methyl)amino)-1-methyl-2-oxo-1,2- dihydro-1,5-naphthyridine-3-carbonitrile |
| 49 | 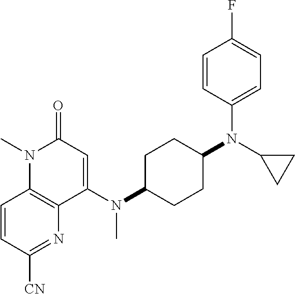 | cis-8-((4-(cyclopropyl(4-fluorophenyl)amino) cyclohexyl)(methyl)amino)-5-methyl-6-oxo-5,6- dihydro-1,5-naphthyridine-2-carbonitrile |
| 50 | 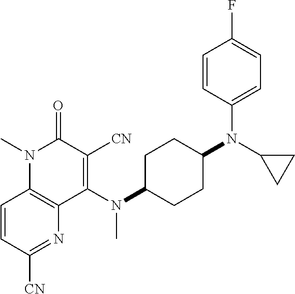 | cis-8-((4-(cyclopropyl(4-fluorophenyl)amino) cyclohexyl)(methyl)amino)-5-methyl-6-oxo-5,6- dihydro-1,5-naphthyridine-2,7-dicarbonitrile |
| 51 | 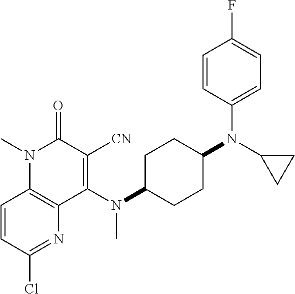 | cis-6-chloro-4-((4-(cyclopropyl(4-fluorophenyl) amino)cyclohexyl)(methyl)amino)-1-methyl-2-oxo- 1,2-dihydro-1,5-naphthyridine-3-carbonitrile |
| 52 | 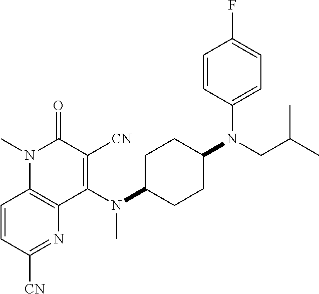 | cis-8-((-4-((4-fluorophenyl)(isobutyl)amino) cyclohexyl)(methyl)amino)-5-methyl-6-oxo-5,6- dihydro-1,5-naphthyridine-2,7-dicarbonitrile |
| 53 | 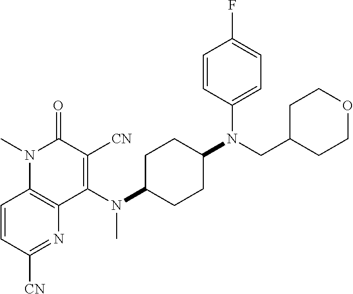 | cis-8-((-4-((4-fluorophenyl)((tetrahydro-2H-pyran-4- yl)methyl)amino)cyclohexyl)(methyl)amino)-5- methyl-6-oxo-5,6-dihydro-1,5-naphthyridine-2,7- dicarbonitrile |
| 54 | 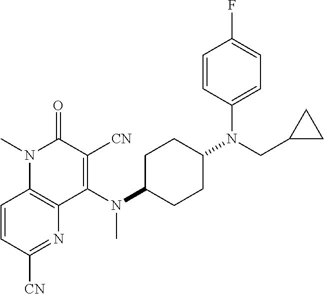 | trans-8-((4-((cyclopropylmethyl)(4-fluorophenyl) amino)cyclohexyl)(methyl)amino)-5-methyl-6-oxo- 5,6-dihydro-1,5-naphthyridine-2,7-dicarbonitrile |
| 55 | 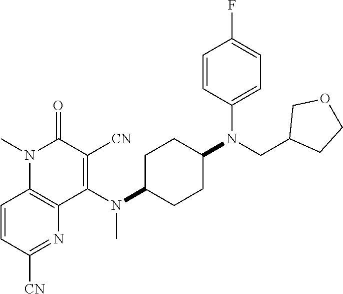 | cis-8-((4-((4-fluorophenyl)((tetrahydrofuran-3-yl) methyl)amino)cyclohexyl)(methyl)amino)-5-methyl- 6-oxo-5,6-dihydro-1,5-naphthyridine-2,7- dicarbonitrile |
| 56 | 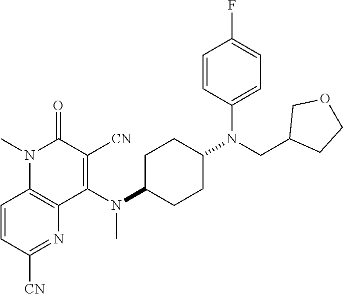 | trans-8-((4-((4-fluorophenyl)((tetrahydrofuran-3-yl) methyl)amino)cyclohexyl)(methyl)amino)-5-methyl- 6-oxo-5,6-dihydro-1,5-naphthyridine-2,7- dicarbonitrile |
| 57 | 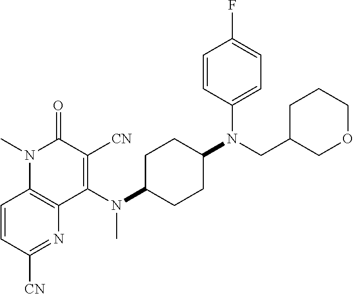 | cis-8-((4-((4-fluorophenyl)((tetrahydro-2H-pyran-3- yl)methyl)amino)cyclohexyl)(methyl)amino)-5- methyl-6-oxo-5,6-dihydro-1,5-naphthyridine-2,7- dicarbonitrile |
| 58 | 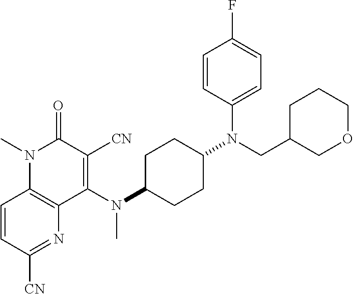 | trans-8-((4-((4-fluorophenyl)((tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 59 | 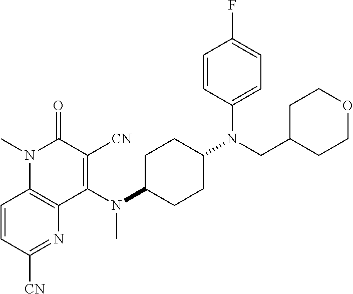 | trans-8-((-4-((4-fluorophenyl)((tetrahydro-2H-pyran- 4-yl)methyl)amino)cyclohexyl)(methyl)amino)-5- methyl-6-oxo-5,6-dihydro-1,5-naphthyridine-2,7- dicarbonitrile |
| 60 | 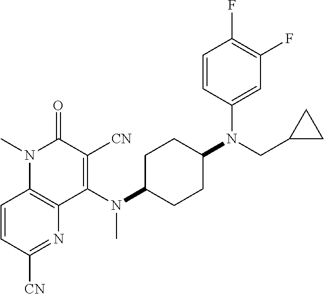 | cis-8-((4-((cyclopropylmethyl)(3,4-difluorophenyl) amino)cyclohexyl)(methyl)amino)-5-methyl-6-oxo- 5,6-dihydro-1,5-naphthyridine-2,7-dicarbonitrile |
| 61 | 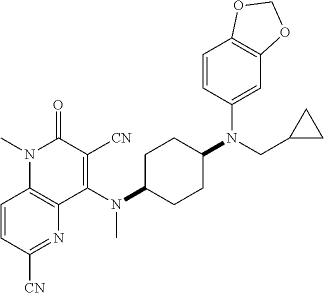 | cis-8-((4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 62 | 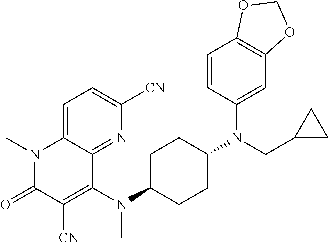 [SMILES: Cn1c(=O)c(C#N)c(N(C)[C@H]2CC[C@H](N(CC3CC3)c3ccc4c(c3)OCO4)CC2)c2nc(C#N)ccc21] | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 63 | 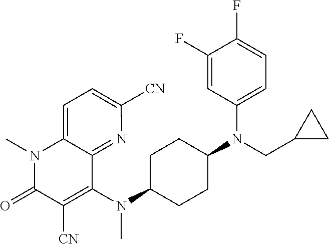 [SMILES: Cn1c(=O)c(C#N)c(N(C)[C@H]2CC[C@@H](N(CC3CC3)c3ccc(F)c(F)c3)CC2)c2nc(C#N)ccc21] | Cis-8-4-((cyclopropylmethyl)(3,4-difluorophenyl) amino)cyclohexyl)(methyl)amino)-5-methyl-6-oxo- 5,6-dihydro-1,5-naphthyridine-2,7-dicarbonitrile |
| 64 | 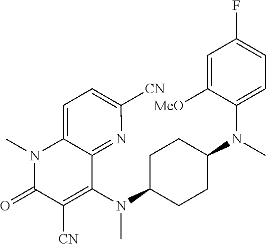 [SMILES: COc1cc(F)ccc1N(C)[C@H]1CC[C@@H](N(C)c2c(C#N)c(=O)n(C)c3ccc(C#N)nc23)CC1] | Cis-8-4-((4-fluoro-2-methoxyphenyl)(methyl)amino) cyclohexyl)(methyl)amino)-5-methyl-6-oxo-5,6- dihydro-1,5-naphthyridine-2,7-dicarbonitrile |
| 65 | 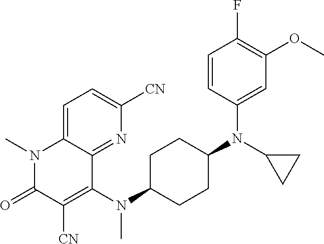 [SMILES: COc1cc(N(C2CC2)[C@H]2CC[C@@H](N(C)c3c(C#N)c(=O)n(C)c4ccc(C#N)nc34)CC2)ccc1F] | cis-8-4-(cyclopropyl(4-fluoro-3-methoxyphenyl) amino)cyclohexyl)(methyl)amino)-5-methyl-6-oxo- 5,6-dihydro-1,5-naphthyridine-2,7-dicarbonitrile |
| 66 | 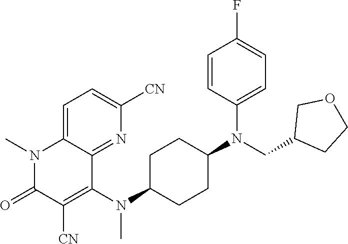 | cis-8-4-((4-fluorophenyl)(((R)-tetrahydrofuran-3-yl) methyl)amino)cyclohexyl)(methyl)amino)-5-methyl- 6-oxo-5,6-dihydro-1,5-naphthyridine-2,7- dicarbonitrile |
| 67 | 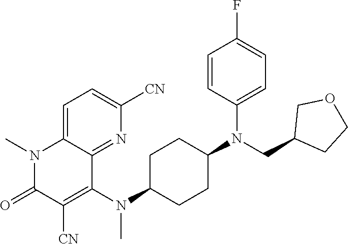 [SMILES: Cn1c(=O)c(C#N)c(N(C)[C@H]2CC[C@@H](N(C[C@@H]3CCOC3)c3ccc(F)cc3)CC2)c2nc(C#N)ccc21] | cis-8-4-((4-fluorophenyl)(((S)-tetrahydrofuran-3-yl) methyl)amino)cyclohexyl)(methyl)amino)-5-methyl- 6-oxo-5,6-dihydro-1,5-naphthyridine-2,7- dicarbonitrile |
| 68 | 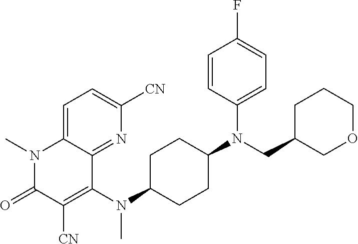 [SMILES: Cn1c(=O)c(C#N)c(N(C)[C@H]2CC[C@@H](N(C[C@H]3CCCOC3)c3ccc(F)cc3)CC2)c2nc(C#N)ccc21] | cis-8-4-((4-fluorophenyl)(((R)-tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 69 | 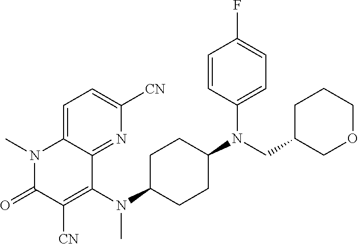 [SMILES: Cn1c(=O)c(C#N)c(N(C)[C@H]2CC[C@@H](N(C[C@@H]3CCCOC3)c3ccc(F)cc3)CC2)c2nc(C#N)ccc21] | cis-8-4-((4-fluorophenyl)(((R)-tetrahydro-2H-pyran- 3-yl)methyl)amino)cyclohexyl)(methyl)amino)-5- methyl-6-oxo-5,6-dihydro-1,5-naphthyridine-2,7- dicarbonitrile |
| 70 | 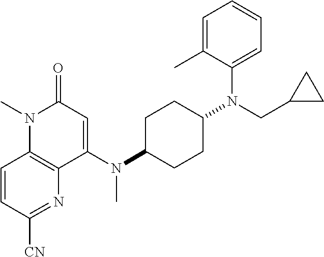 | trans-8-((4-((cyclopropylmethyl)(o-tolyl)amino) cyclohexyl)(methyl)amino)-5-methyl-6-oxo-5,6- dihydro-1,5-naphthyridine-2-carbonitrile |
| 71 | 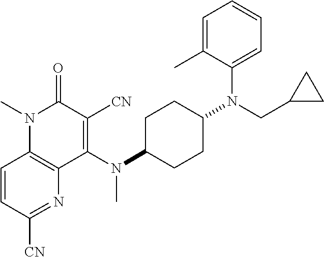 | trans-8-((4-((cyclopropylmethyl)(o-tolyl)amino) cyclohexyl)(methyl)amino)-5-methyl-6-oxo-5,6- dihydro-1,5-naphthyridine-2,7-dicarbonitrile |
| 72 | 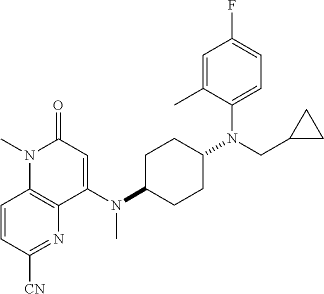 | trans-8-((4-((cyclopropylmethyl)(4-fluoro-2- methylphenyl)amino)cyclohexyl)(methyl)amino)-5- methyl-6-oxo-5,6-dihydro-1,5-naphthyridine-2- carbonitrile |
| 73 | 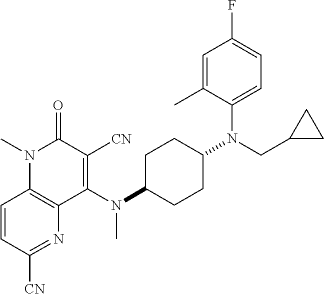 | trans-8-((4-((cyclopropylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 74 | 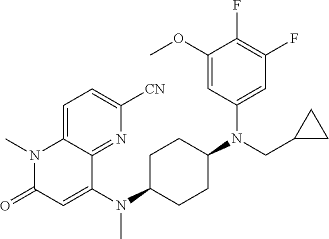 [SMILES: COc1cc(N(CC2CC2)[C@H]2CC[C@@H](N(C)c3cc(=O)n(C)c4ccc(C#N)nc34)CC2)cc(F)c1F] | cis-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2- carbonitrile |
| 75 | 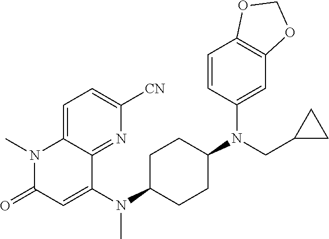 [SMILES: Cn1c(=O)cc(N(C)[C@H]2CC[C@@H](N(CC3CC3)c3ccc4c(c3)OCO4)CC2)c2nc(C#N)ccc21] | cis-8-4-(benzo[d][1,3]dioxol-5-yl(cyclopropylmethyl) amino)cyclohexyl)(methyl)amino)-5-methyl-6-oxo- 5,6-dihydro-1,5-naphthyridine-2-carbonitrile |
| 76 | 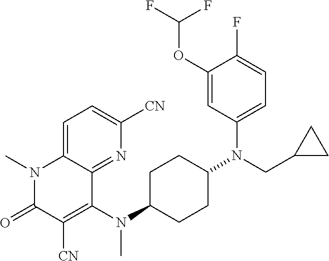 [SMILES: Cn1c(=O)c(C#N)c(N(C)[C@H]2CC[C@H](N(CC3CC3)c3ccc(F)c(OC(F)F)c3)CC2)c2nc(C#N)ccc21] | trans-8-4-((cyclopropylmethyl)(3-(difluoromethoxy)- 4-fluorophenyl)amino)cyclohexyl)(methyl)amino)-5- methyl-6-oxo-5,6-dihydro-1,5-naphthyridine-2,7- dicarbonitrile |
| 77 | 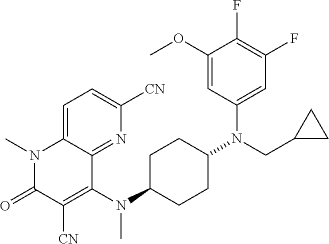 [SMILES: COc1cc(N(CC2CC2)[C@H]2CC[C@H](N(C)c3c(C#N)c(=O)n(C)c4ccc(C#N)nc34)CC2)cc(F)c1F] | trans-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2,7- dicarbonitrile |
| 78 | 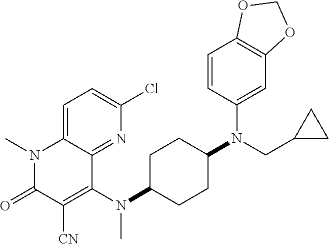 | cis-4-4-(benzo[d][1,3]dioxol-5-yl(cyclopropylmethyl) amino)cyclohexyl)(methyl)amino)-6-chloro-1- methyl-2-oxo-1,2-dihydro-1,5-naphthyridine-3- carbonitrile |
| 79 | 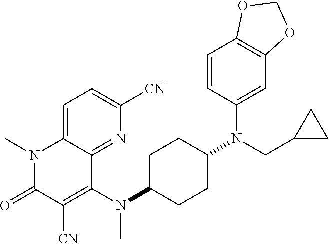 | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2,7-dicarbonitrile |
| 80 | 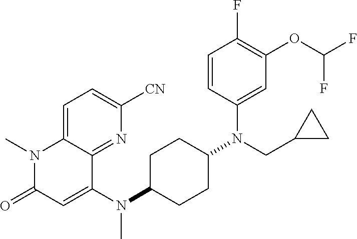 | trans-8-4-((cyclopropylmethyl)(3-(difluoromethoxy)- 4-fluorophenyl)amino)cyclohexyl)(methyl)amino)-5- methyl-6-oxo-5,6-dihydro-1,5-naphthyridine-2- carbonitrile |
| 81 | 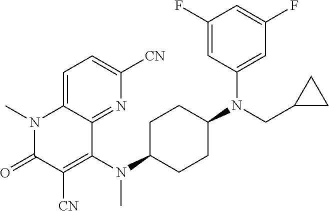 [SMILES: Cn1c(=O)c(C#N)c(N(C)[C@H]2CC[C@@H](N(CC3CC3)c3cc(F)cc(F)c3)CC2)c2nc(C#N)ccc21] | cis-8-4-((cyclopropylmethyl)(3,5-difluorophenyl) amino)cyclohexyl)(methyl)amino)-5-methyl-6-oxo- 5,6-dihydro-1,5-naphthyridine-2,7-dicarbonitrile |
| 82 | 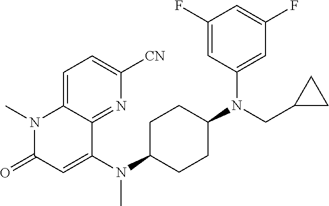 [SMILES: Cn1c(=O)cc(N(C)[C@H]2CC[C@@H](N(CC3CC3)c3cc(F)cc(F)c3)CC2)c2nc(C#N)ccc21] | cis-8-4-((cyclopropylmethyl)(3,5-difluorophenyl) amino)cyclohexyl)(methyl)amino)-5-methyl-6-oxo- 5,6-dihydro-1,5-naphthyridine-2-carbonitrile |
| 83 | 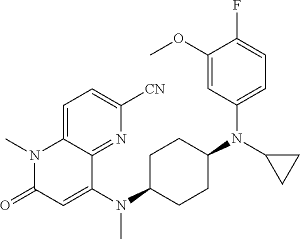 [SMILES: COc1cc(N(C2CC2)[C@H]2CC[C@@H](N(C)c3cc(=O)n(C)c4ccc(C#N)nc34)CC2)ccc1F] | cis-8-4-(cyclopropyl(4-fluoro-3-methoxyphenyl) amino)cyclohexyl)(methyl)amino)-5-methyl-6-oxo- 5,6-dihydro-1,5-naphthyridine-2-carbonitrile |
| 84 | 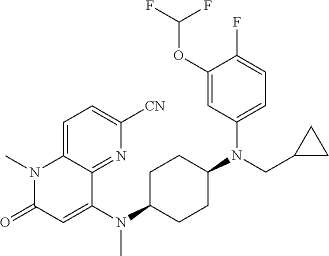 [SMILES: Cn1c(=O)cc(N(C)[C@H]2CC[C@@H](N(CC3CC3)c3ccc(F)c(OC(F)F)c3)CC2)c2nc(C#N)ccc21] | cis-8-4-((cyclopropylmethyl)(3-(difluoromethoxy)-4- fluorophenyl)amino)cyclohexyl)(methyl)amino)-5- methyl-6-oxo-5,6-dihydro-1,5-naphthyridine-2- carbonitrile |
| 85 | 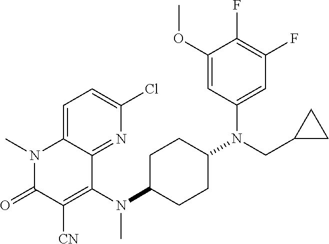 | trans-6-chloro-4-4-((cyclopropylmethyl)(3,4- difluoro-5-methoxyphenyl)amino)cyclohexyl) (methyl)amino)-1-methyl-2-oxo-1,2-dihydro-1,5- naphthyridine-3-carbonitrile |
| 86 | 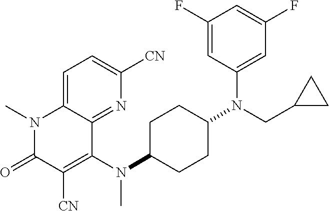 | trans-8-4-((cyclopropylmethyl)(3,5-difluorophenyl) amino)cyclohexyl)(methyl)amino)-5-methyl-6-oxo- 5,6-dihydro-1,5-naphthyridine-2,7-dicarbonitrile |
| 87 | 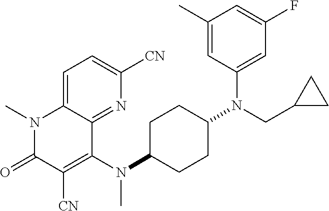 | trans-8-4-((cyclopropylmethyl)(3-fluoro-5- methylphenyl)amino)cyclohexyl)(methyl)amino)-5- methyl-6-oxo-5,6-dihydro-1,5-naphthyridine-2,7- dicarbonitrile |
| 88 | 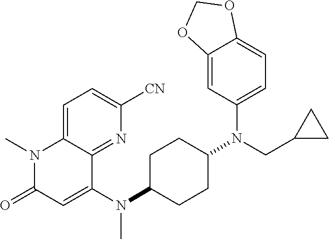 | trans-8-4-(benzo[d][1,3]dioxol-5- yl(cyclopropylmethyl)amino)cyclohexyl)(methyl) amino)-5-methyl-6-oxo-5,6-dihydro-1,5- naphthyridine-2-carbonitrile |
| 89 | 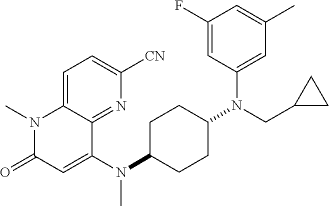 | trans-8-4-((cyclopropylmethyl)(3-fluoro-5- methylphenyl)amino)cyclohexyl)(methyl)amino)-5- methyl-6-oxo-5,6-dihydro-1,5-naphthyridine-2- carbonitrile |
| 90 | 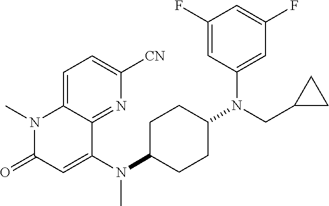 | trans-8-4-((cyclopropylmethyl)(3,5-difluorophenyl) amino)cyclohexyl)(methyl)amino)-5-methyl-6-oxo- 5,6-dihydro-1,5-naphthyridine-2-carbonitrile |
| 91 | 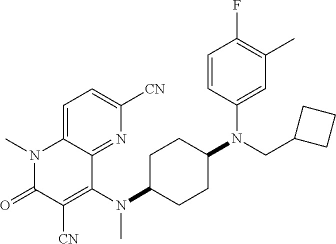 | cis-8-4-((cyclobutylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 92 | 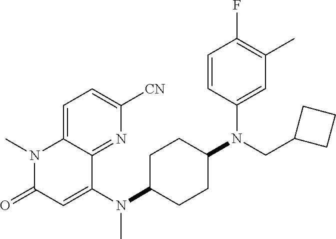 | cis-8-4-((cyclobutylmethyl)(4-fluoro-3- methyl-6-oxo-5,6-dihydro-1,5-naphthyridine-2- methylphenyl)amino)cyclohexyl)(methyl)amino)-5- carbonitrile |
| 93 | 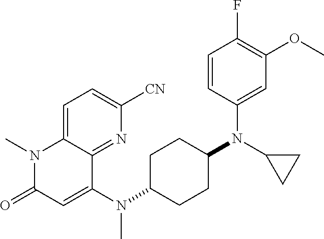 | trans-8-4-(cyclopropyl(4-fluoro-3-methoxyphenyl) amino)cyclohexyl)(methyl)amino)-5-methyl-6-oxo- 5,6-dihydro-1,5-naphthyridine-2-carbonitrile |
| 94 | 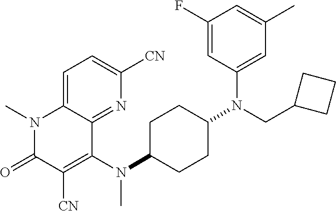 | trans-8-4-((cyclobutylmethy])(3-fluoro-5- methylphenyl)amino)cyclohexyl)(methyl)amino)-5- methyl-6-oxo-5,6-dihydro-1,5-naphthyridine-2,7- dicarbonitrile |
| 95 | 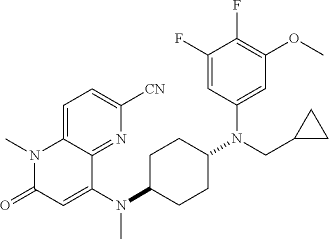 | trans-8-4-((cyclopropylmethyl)(3,4-difluoro-5- methoxyphenyl)amino)cyclohexyl)(methyl)amino)-5- methyl-6-oxo-5,6-dihydro-1,5-naphthyridine-2- carbonitrile |
| 96 | 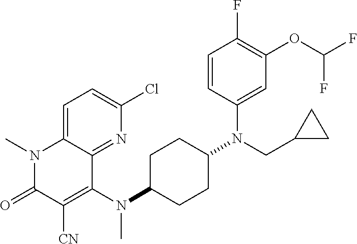 | trans-6-chloro-4-((cyclopropylmethyl)(3- (difluoromethoxy)-4-fluorophenyl)amino)cyclohexyl) (methyl)amino)-1-methyl-2-oxo-1,2-dihydro-1,5- naphthyridine-3-carbonitrile |
| 97 | 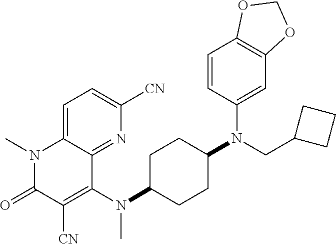 | cis-8-4-(benzo[d][1,3]dioxol-5-yl(cyclobutylmethyl) amino)cyclohexyl)(methyl)amino)-5-methyl-6-oxo- 5,6-dihydro-1,5-naphthyridine-2,7-dicarbonitrile |
| 98 | 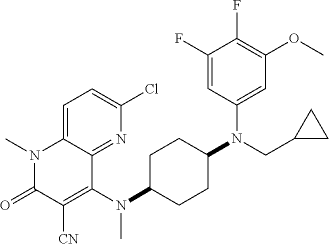 | cis-6-chloro-4-4-((cyclopropylmethyl)(3,4-difluoro-5- methoxyphenyl)amino)cyclohexyl)(methyl)amino)-1- methyl-2-oxo-1,2-dihydro-1,5-naphthyridine-3- carbonitrile |
| 99 | 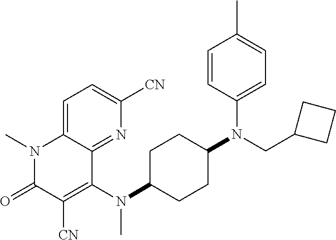 | cis-8-4-((cyclobutylmethyl)(p-tolyl)amino) cyclohexyl)(methyl)amino)-5-methyl-6-oxo-5,6- dihydro-1,5-naphthyridine-2,7-dicarbonitrile |
| 100 | 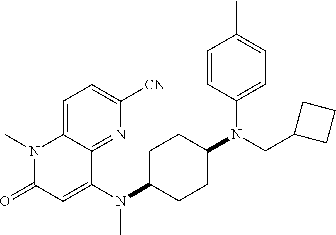 | cis-8-4-((cyclobutylmethy])(p-tolyl)amino) cyclohexyl)(methyl)amino)-5-methyl-6-oxo-5,6- dihydro-1,5-naphthyridine-2-carbonitrile |
| 101 | 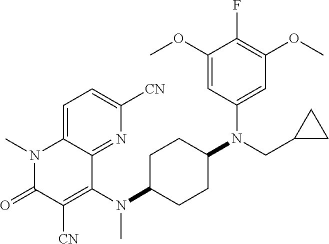 | cis-8-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2,7- dicarbonitrile |
| 102 | 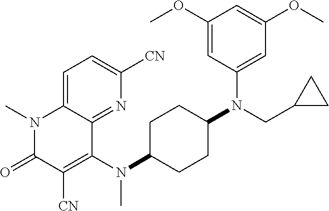 | cis-8-4-((cyclopropylmethyl)(3,5-dimethoxyphenyl) amino)cyclohexyl)(methyl)amino)-5-methyl-6-oxo- 5,6-dihydro-1,5-naphthyridine-2,7-dicarbonitrile |
| 103 | 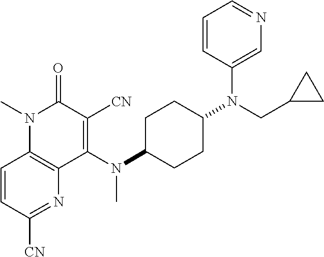 | trans-8-((4-((cyclopropylmethyl)(pyridin-3-yl)amino) cyclohexyl)(methyl)amino)-5-methyl-6-oxo-5,6- dihydro-1,5-naphthyridine-2,7-dicarbonitrile |
| 104 | 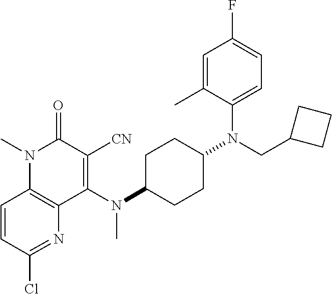 | trans-6-chloro-4-((4-((cyclobutylmethyl)(4-fluoro-2- methylphenyl)amino)cyclohexyl)(methyl)amino)-1- methyl-2-oxo-1,2-dihydro-1,5-naphthyridine-3- carbonitrile |
| 105 | 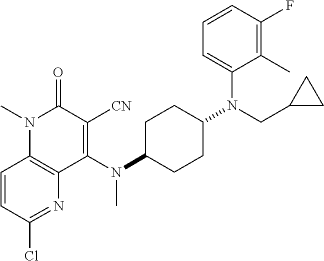 | trans-6-chloro-4-((4-((cyclopropylmethyl)(3-fluoro- 2-methylphenyl)amino)cyclohexyl)(methyl)amino)-1- methyl-2-oxo-1,2-dihydro-1,5-naphthyridine-3- carbonitrile |
| 106 | 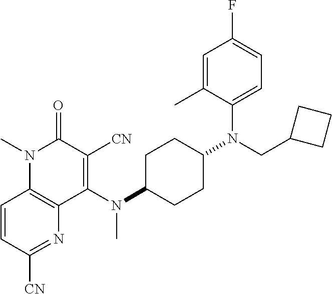 | trans-8-((4-((cyclobutylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 107 | 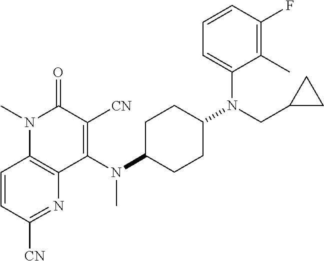 | trans-8-((4-((cyclopropylmethyl)(3-fluoro-2- methylphenyl)amino)cyclohexyl)(methyl)amino)-5- methyl-6-oxo-5,6-dihydro-1,5-naphthyridine-2,7- dicarbonitrile |
| 108 | 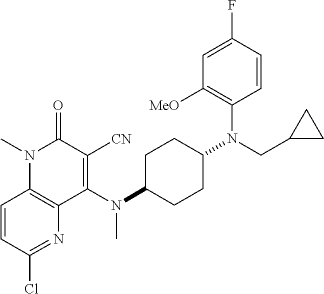 | trans-6-chloro-4-((-4-((cyclopropylmethyl)(4-fluoro- 2-methoxyphenyl)amino)cyclohexyl)(methyl)amino)- 1-methyl-2-oxo-1,2-dihydro-1,5-naphthyridine-3- carbonitrile |
| 109 | 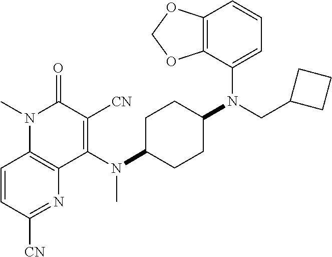 | cis-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2,7-dicarbonitrile |
| 110 | 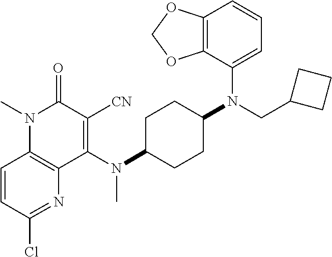 | cis-4-((-4-(benzo[d][1,3]dioxol-4- yl(cyclobutylmethyl)amino)cyclohexyl)(methyl) amino)-6-chloro-1-methyl-2-oxo-1,2-dihydro-1,5- naphthyridine-3-carbonitrile |
| 111 | 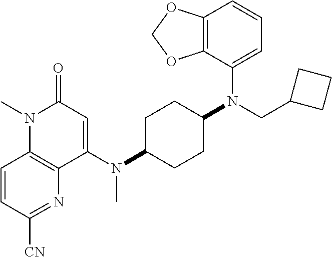 | cis-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2-carbonitrile |
| 112 | 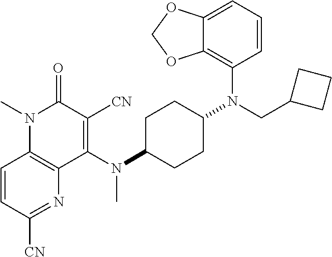 | trans-8-((-4-(benzo[d][1,3]dioxol-4- yl(cyclobutylmethyl)amino)cyclohexyl)(methyl) amino)-5-methyl-6-oxo-5,6-dihydro-1,5- naphthyridine-2,7-dicarbonitrile |
| 113 | 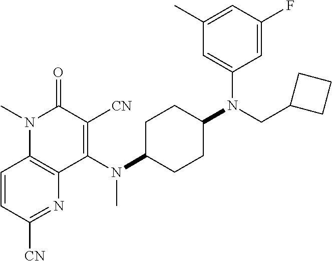 | cis-8-((-4-((cyclobutylmethyl)(3-fluoro-5- methylphenyl)amino)cyclohexyl)(methyl)amino)-5- methyl-6-oxo-5,6-dihydro-1,5-naphthyridine-2,7- dicarbonitrile |
| 114 | 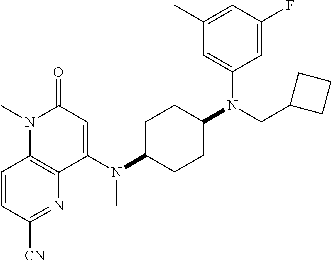 | cis-8-((-4-((cyclobutylmethyl)(3-fluoro-5- methylphenyl)amino)cyclohexyl)(methyl)amino)-5- methyl-6-oxo-5,6-dihydro-1,5-naphthyridine-2- carbonitrile |
| 115 | 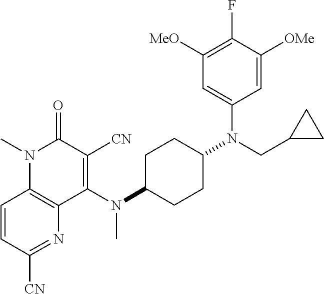 | trans-8-((-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2,7- dicarbonitrile |
| 116 | 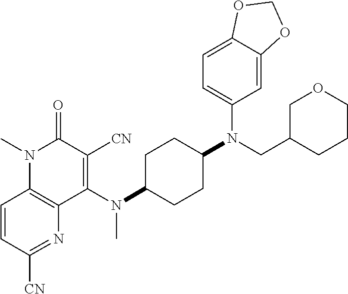 | cis-8-((-4-(benzo[d][1,3]dioxol-5-yl((tetrahydro-2H- pyran-3-yl)methyl)amino)cyclohexyl)(methyl) amino)-5-methyl-6-oxo-5,6-dihydro-1,5- naphthyridine-2,7-dicarbonitrile |
| 117 | 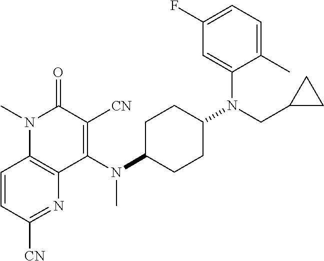 | trans-8-((4-((cyclopropylmethyl)(5-fluoro-2- methylphenyl)amino)cyclohexyl)(methyl)amino)-5- methyl-6-oxo-5,6-dihydro-1,5-naphthyridine-2,7- dicarbonitrile |
| 118 | 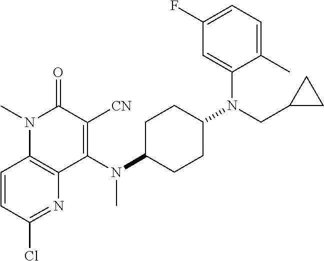 | trans-6-chloro-4-((4-((cyclopropylmethyl)(5-fluoro- 2-methylphenyl)amino)cyclohexyl)(methyl)amino)-1- methyl-2-oxo-1,2-dihydro-1,5-naphthyridine-3- carbonitrile |
| 119 | 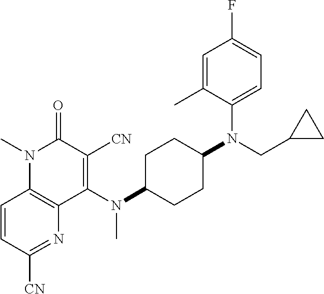 | cis-8-((4-((cyclopropylmethyl)(4-fluoro-2- methylphenyl)amino)cyclohexyl)(methyl)amino)-5- methyl-6-oxo-5,6-dihydro-1,5-naphthyridine-2,7- dicarbonitrile |
| 120 | 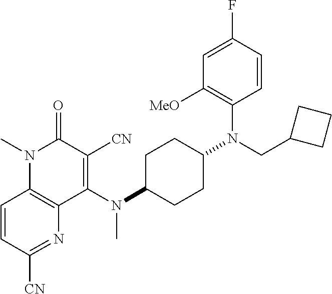 | trans-8-((-4-((cyclobutylmethyl)(4-fluoro-2- methoxyphenyl)amino)cyclohexyl)(methyl)amino)-5- methyl-6-oxo-5,6-dihydro-1,5-naphthyridine-2,7- dicarbonitrile |
| 121 | 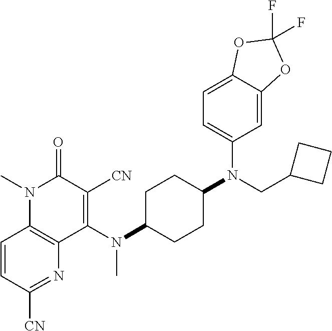 | cis-8-((-4-((cyclobutylmethyl)(2,2-difluorobenzo[d] [1,3]dioxol-5-yl)amino)cyclohexyl)(methyl)amino)- 5-methyl-6-oxo-5,6-dihydro-1,5-naphthyridine-2,7- dicarbonitrile |
| 122 | 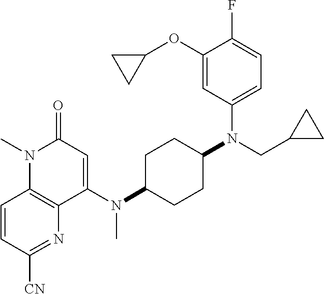 | cis-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2-carbonitrile |
| 123 | 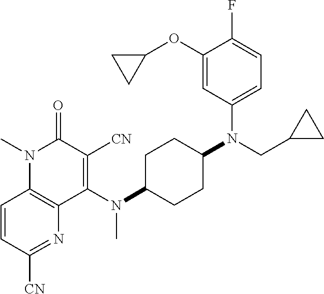 | cis-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2,7-dicarbonitrile |
| 124 | 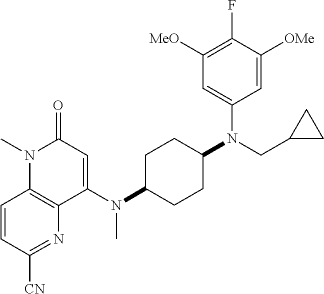 | cis-8-((-4-((cyclopropylmethyl)(4-fluoro-3,5- dimethoxyphenyl)amino)cyclohexyl)(methyl)amino)- 5-methyl-6-oxo-5,6-dihydro-1,5-naphthyridine-2- carbonitrile |
| 125 | 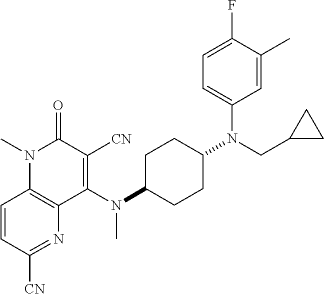 | trans-8-((4-((cyclopropylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 126 | 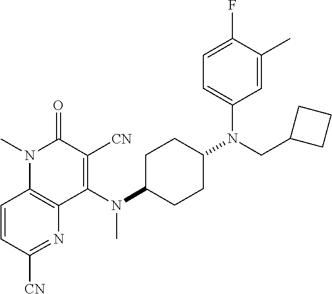 | trans-8-((-4-((cyclobutylmethyl)(4-fluoro-3- methylphenyl)amino)cyclohexyl)(methyl)amino)-5- methyl-6-oxo-5,6-dihydro-1,5-naphthyridine-2,7- dicarbonitrile |
| 127 | 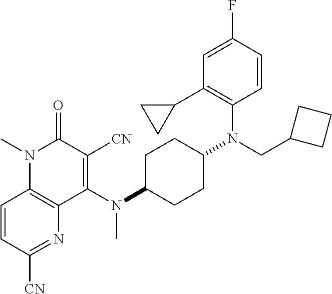 | trans-8-((-4-((cyclobutylmethyl)(2-cyclopropyl-4- fluorophenyl)amino)cyclohexyl)(methyl)amino)-5- methyl-6-oxo-5,6-dihydro-1,5-naphthyridine-2,7- dicarbonitrile |
| 128 | 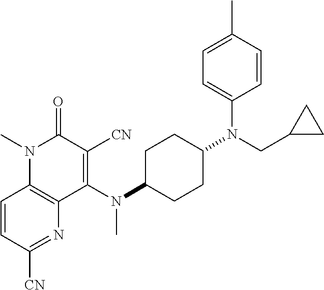 | trans-8-((-4-((cyclopropylmethyl)(p-toly])amino) cyclohexyl)(methyl)amino)-5-methyl-6-oxo-5,6- dihydro-1,5-naphthyridine-2,7-dicarbonitrile |
| 129 | 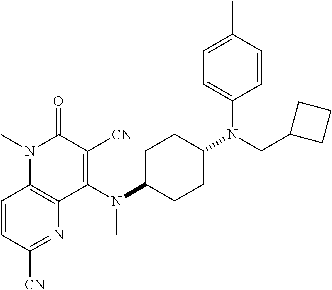 | trans-8-((-4-((cyclobutylmethyl)(p-tolyl)amino) cyclohexyl)(methyl)amino)-5-methyl-6-oxo-5,6- dihydro-1,5-naphthyridine-2,7-dicarbonitrile |
| 130 | 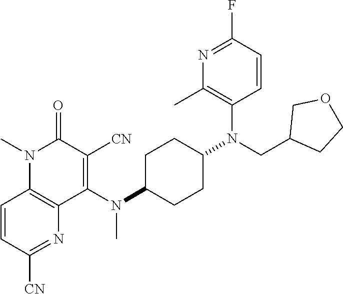 | trans-8-((-4-((6-fluoro-2-methylpyridin-3-yl) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 131 | 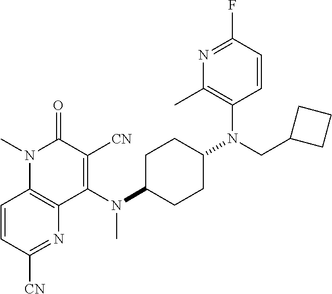 | trans-8-((-4-((cyclobutylmethyl)(6-fluoro-2- methylpyridin-3-yl)amino)cyclohexyl)(methyl) amino)-5-methyl-6-oxo-5,6-dihydro-1,5- naphthyridine-2,7-dicarbonitrile |
| 132 | 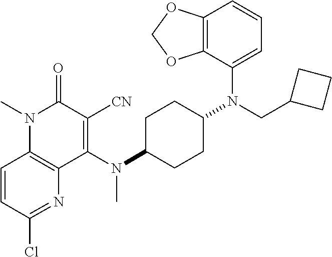 | trans-4-((-4-(benzo[d][1,3]dioxol-4- yl(cyclobutylmethyl)amino)cyclohexyl)(methyl) amino)-6-chloro-1-methyl-2-oxo-1,2-dihydro-1,5- naphthyridine-3-carbonitrile |
| 133 | 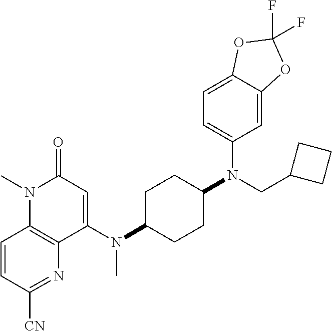 | cis-8-((-4-((cyclobutylmethyl)(2,2-difluorobenzo[d] [1,3]dioxol-5-yl)amino)cyclohexyl)(methyl)amino)- 5-methyl-6-oxo-5,6-dihydro-1,5-naphthyridine-2- carbonitrile |
| 134 | 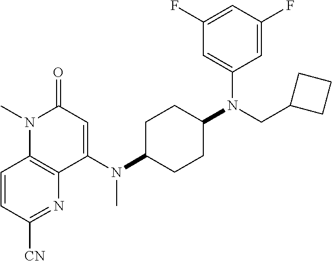 | cis-8-((-4-((cyclobutylmethyl)(3,5-difluorophenyl) amino)cyclohexyl)(methyl)amino)-5-methyl-6-oxo- 5,6-dihydro-1,5-naphthyridine-2-carbonitrile |
| 135 | 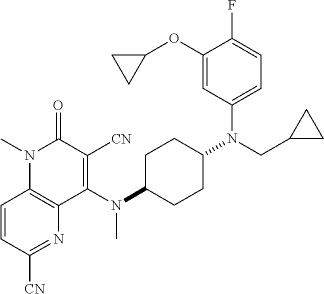 | trans-8-((-4-((3-cyclopropoxy-4-fluorophenyl) (cyclopropylmethyl)amino)cyclohexyl)(methyl) amino)-5-methyl-6-oxo-5,6-dihydro-1,5- naphthyridine-2,7-dicarbonitrile |
| 136 | 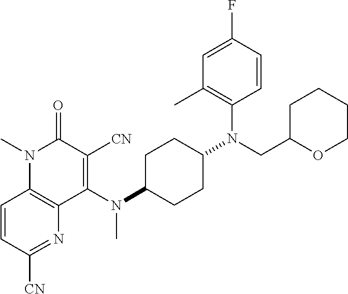 | trans-8-((-4-((4-fluoro-2-methylpheny])((tetrahydro- 2H-pyran-2-yl)methyl)amino)cyclohexyl)(methyl) amino)-5-methyl-6-oxo-5,6-dihydro-1,5- naphthyridine-2,7-dicarbonitrile |
| 137 | 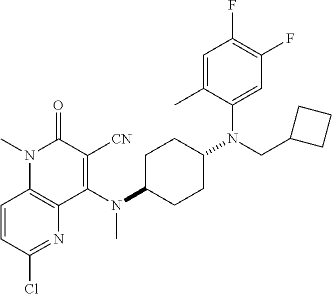 | trans-6-chloro-4-((4-((cyclobutylmethyl)(4,5- difluoro-2-methylphenyl)amino)cyclohexyl)(methyl) amino)-1-methyl-2-oxo-1,2-dihydro-1,5- naphthyridine-3-carbonitrile |
| 138 | 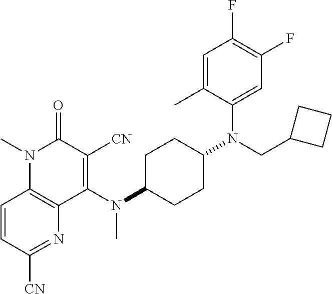 | trans-8-((4-((cyclobutylmethyl)(4,5-difluoro-2- methylphenyl)amino)cyclohexyl)(methyl)amino)-5- methyl-6-oxo-5,6-dihydro-1,5-naphthyridine-2,7- dicarbonitrile |
| 139 | 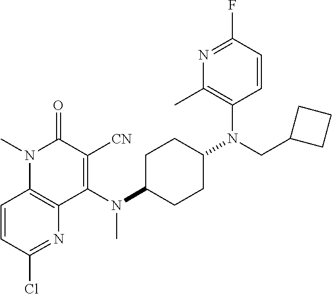 | trans-6-chloro-4-((-4-((cyclobutylmethyl)(6-fluoro-2- methylpyridin-3-yl)amino)cyclohexyl)(methyl) amino)-1-methyl-2-oxo-1,2-dihydro-1,5- naphthyridine-3-carbonitrile |
| 140 | 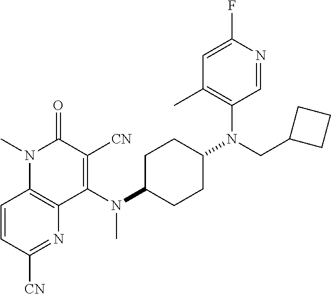 | trans-8-((-4-((cyclobutylmethyl)(6-fluoro-4- methylpyridin-3-yl)amino)cyclohexyl)(methyl) amino)-5-methyl-6-oxo-5,6-dihydro-1,5- naphthyridine-2,7-dicarbonitrile |
| 14 | 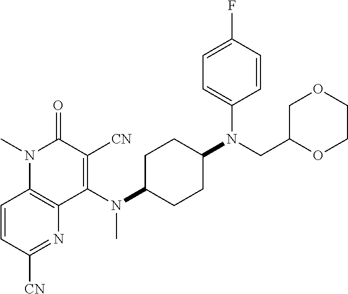 | cis-8-((-4-(((1,4-dioxan-2-yl)methyl)(4-fluorophenyl) amino)cyclohexyl)(methyl)amino)-5-methyl-6-oxo- 5,6-dihydro-1,5-naphthyridine-2,7-dicarbonitrile |
| 142 | 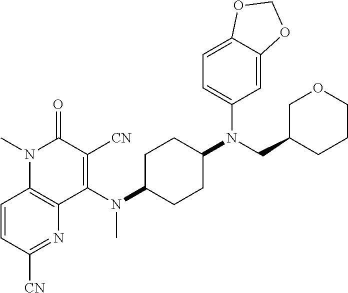 | cis-8-((-4-(benzo[d][1,3]dioxol-5-yl(((S)-tetrahydro- 2H-pyran-3-yl)methyl)amino)cyclohexyl)(methyl) amino)-5-methyl-6-oxo-5,6-dihydro-1,5- naphthyridine-2,7-dicarbonitrile |
| 143 | 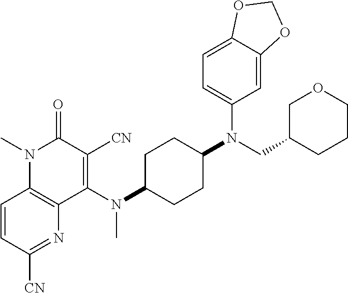 | cis-8-((-4-(benzo[d][1,3]dioxol-5-yl(((R)-tetrahydro- 2H-pyran-3-yl)methyl)amino)cyclohexyl)(methyl) amino)-5-methyl-6-oxo-5,6-dihydro-1,5- naphthyridine-2,7-dicarbonitrile |
| 144 | 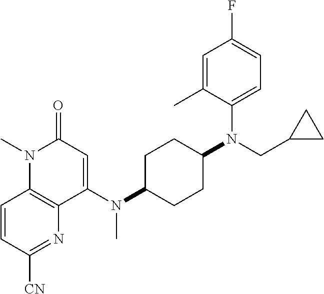 | cis-8-((4-((cyclopropylmethyl)(4-fluoro-2- methylphenyl)amino)cyclohexyl)(methyl)amino)-5- methyl-6-oxo-5,6-dihydro-1,5-naphthyridine-2- carbonitrile |
| 145 | 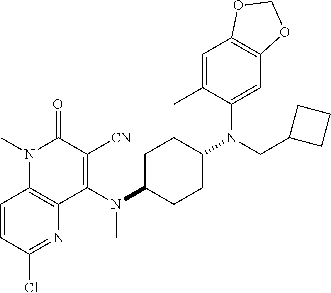 | trans-6-chloro-4-((4-((cyclobutylmethy])(6- methylbenzo[d][1,3]dioxol-5-yl)amino)cyclohexyl) (methyl)amino)-1-methyl-2-oxo-1,2-dihydro-1,5- naphthyridine-3-carbonitrile |
| 146 | 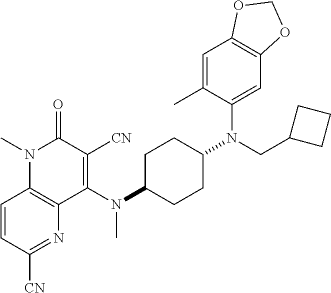 | trans-8-((4-((cyclobutylmethyl)(6-methylbenzo[d] [1,3]dioxol-5-yl)amino)cyclohexyl)(methyl)amino)- 5-methyl-6-oxo-5,6-dihydro-1,5-naphthyridine-2,7- dicarbonitrile |
| 147 | 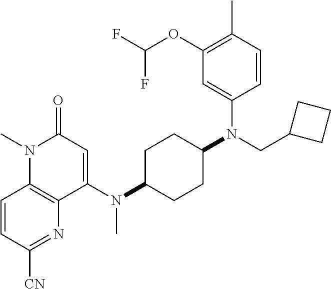 | cis-8-((-4-((cyclobutylmethyl)(3-(difluoromethoxy)- 4-methylphenyl)amino)cyclohexyl)(methyl)amino)-5- methyl-6-oxo-5,6-dihydro-1,5-naphthyridine-2- carbonitrile |
| 148 | 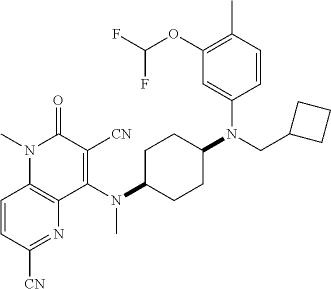 | cis-8-((-4-((cyclobutylmethyl)(3-(difluoromethoxy)- 4-methylphenyl)amino)cyclohexyl)(methyl)amino)-5- methyl-6-oxo-5,6-dihydro-1,5-naphthyridine-2,7- dicarbonitrile |
| 149 | 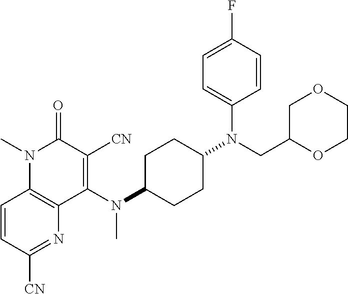 | trans-8-((-4-(((1,4-dioxan-2-yl)methyl)(4- fluorophenyl)amino)cyclohexyl)(methyl)amino)-5- methyl-6-oxo-5,6-dihydro-1,5-naphthyridine-2,7- dicarbonitrile |
| 150 | 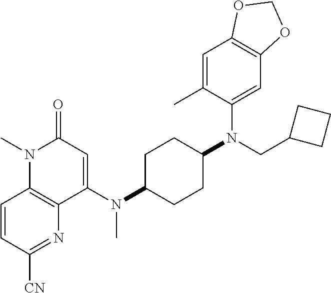 | cis-8-((4-((cyclobutylmethy])(6-methylbenzo[d][1,3] dioxol-5-yl)amino)cyclohexyl)(methyl)amino)-5- methyl-6-oxo-5,6-dihydro-1,5-naphthyridine-2- carbonitrile |
| 151 | 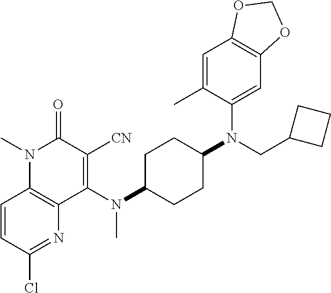 | cis-6-chloro-4-((4-((cyclobutylmethyl)(6- methylbenzo[d][1,3]dioxol-5-yl)amino)cyclohexyl) (methyl)amino)-1-methyl-2-oxo-1,2-dihydro-1,5- naphthyridine-3-carbonitrile |
| 152 | 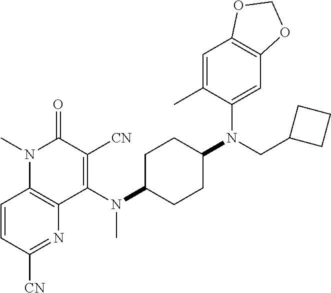 | cis-8-((4-((cyclobutylmethyl)(6-methylbenzo[d][1,3] dioxol-5-yl)amino)cyclohexyl)(methyl)amino)-5- methyl-6-oxo-5,6-dihydro-1,5-naphthyridine-2,7- dicarbonitrile |
| 153 | 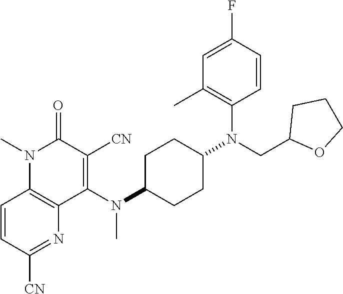 | cis-8-((-4-((4-fluoro-2-methylphenyl) ((tetrahydrofuran-2-yl)methyl)amino)cyclohexyl) (methyl)amino)-5-methyl-6-oxo-5,6-dihydro-1,5- naphthyridine-2,7-dicarbonitrile |
| 154 | 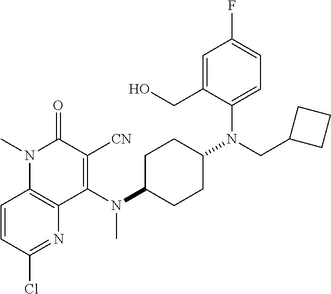 | trans-6-chloro-4-((4-((cyclobutylmethyl)(4-fluoro-2- (hydroxymethyl)phenyl)amino)cyclohexyl)(methyl) amino)-1-methyl-2-oxo-1,2-dihydro-1,5- naphthyridine-3-carbonitrile |
| 155 | 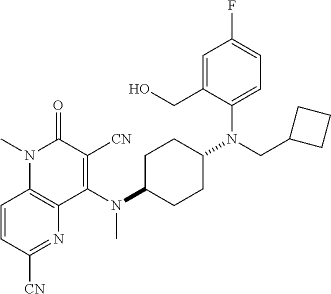 | trans-8-((4-((cyclobutylmethyl)(4-fluoro-2- (hydroxymethyl)phenyl)amino)cyclohexyl)(methyl) amino)-5-methyl-6-oxo-5,6-dihydro-1,5- naphthyridine-2,7-dicarbonitrile |
| 156 | 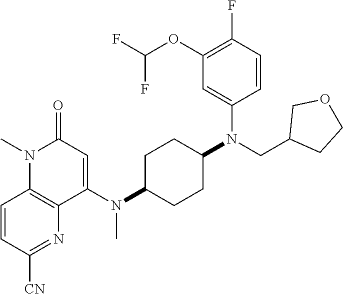 | cis-8-((-4-((3-(difluoromethoxy)-4-fluorophenyl) ((tetrahydrofuran-3-yl)methyl)amino)cyclohexyl) (methyl)amino)-5-methyl-6-oxo-5,6-dihydro-1,5- naphthyridine-2-carbonitrile |
| 157 | 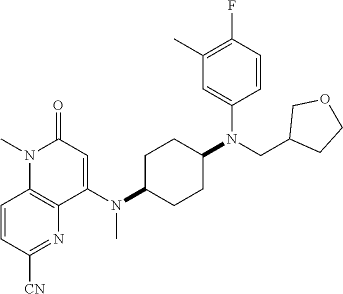 | cis-8-((-4-((4-fluoro-3-methylpheny]) ((tetrahydrofuran-3-yl)methyl)amino)cyclohexyl) (methyl)amino)-5-methyl-6-oxo-5,6-dihydro-1,5- naphthyridine-2-carbonitrile |
| 158 | 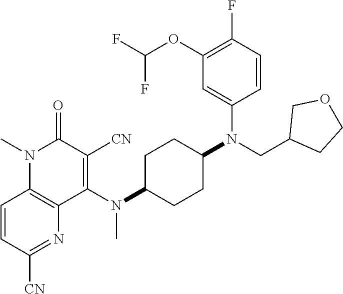 | cis-8-((-4-((3-(difluoromethoxy)-4-fluorophenyl) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 159 | 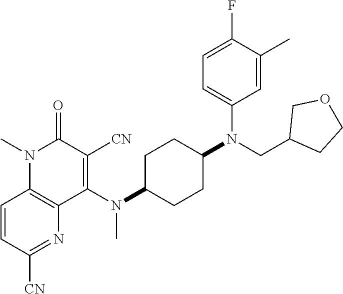 | cis-8-((-4-((4-fluoro-3-methylpheny]) ((tetrahydrofuran-3-yl)methyl)amino)cyclohexyl) (methyl)amino)-5-methyl-6-oxo-5,6-dihydro-1,5- naphthyridine-2,7-dicarbonitrile |
| 160 | 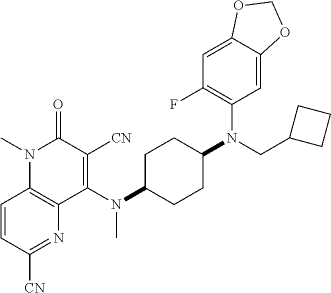 | cis-8-((-4-((cyclobutylmethyl)(6-fluorobenzo[d][1,3] dioxol-5-yl)amino)cyclohexyl)(methyl)amino)-5- methyl-6-oxo-5,6-dihydro-1,5-naphthyridine-2,7- dicarbonitrile |
| 161 | 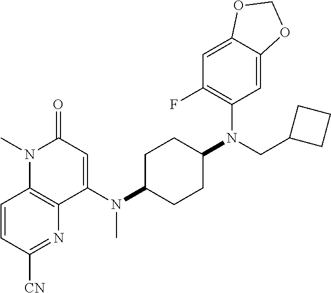 | cis-8-((-4-((cyclobutylmethyl)(6-fluorobenzo[d][1,3] dioxol-5-yl)amino)cyclohexyl)(methyl)amino)-5- methyl-6-oxo-5,6-dihydro-1,5-naphthyridine-2- carbonitrile |
| 162 | 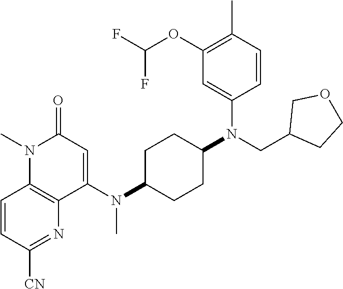 | cis-8-((-4-((3-(difluoromethoxy)-4-methylphenyl) ((tetrahydrofuran-3-yl)methyl)amino)cyclohexyl) (methyl)amino)-5-methyl-6-oxo-5,6-dihydro-1,5- naphthyridine-2-carbonitrile |
| 163 | 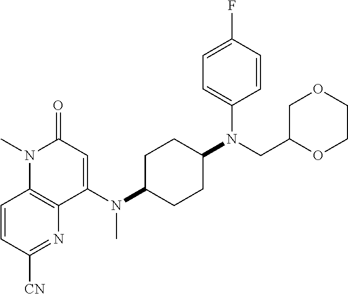 | cis-8-((-4-(((1,4-dioxan-2-yl)methyl)(4-fluorophenyl) amino)cyclohexyl)(methyl)amino)-5-methyl-6-oxo- 5,6-dihydro-1,5-naphthyridine-2-carbonitrile |
| 164 | 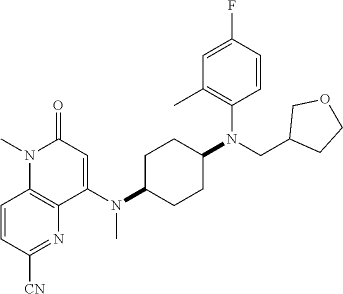 | cis-8-((-4-((4-fluoro-2-methylpheny]) ((tetrahydrofuran-3-yl)methyl)amino)cyclohexyl) (methyl)amino)-5-methyl-6-oxo-5,6-dihydro-1,5- naphthyridine-2-carbonitrile |
| 165 | 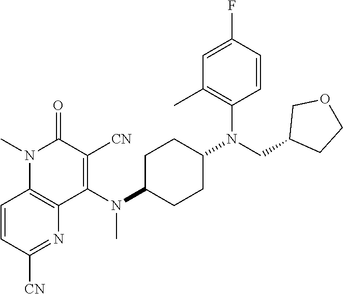 | trans-8-((-4-((4-fluoro-2-methylphenyl)(((R)- tetrahydrofuran-3-yl)methyl)amino)cyclohexyl) (methyl)amino)-5-methyl-6-oxo-5,6-dihydro-1,5- naphthyridine-2,7-dicarbonitrile |
| 166 | 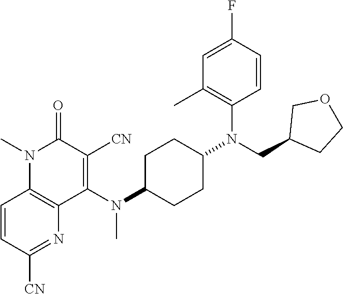 | trans-8-((-4-((4-fluoro-2-methylpheny])(((S)- tetrahydrofuran-3-yl)methyl)amino)cyclohexyl) (methyl)amino)-5-methyl-6-oxo-5,6-dihydro-1,5- naphthyridine-2,7-dicarbonitrile |
| 167 | 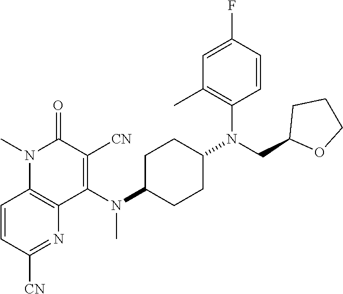 | trans-8-((-4-((4-fluoro-2-methylpheny])(((R)- tetrahydrofuran-2-yl)methyl)amino)cyclohexyl) (methyl)amino)-5-methyl-6-oxo-5,6-dihydro-1,5- naphthyridine-2,7-dicarbonitrile |
| 168 | 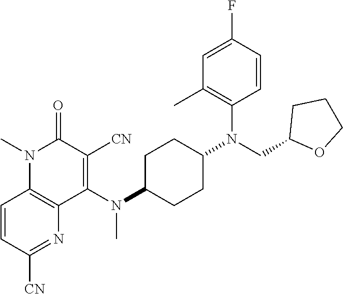 | trans-8-((-4-((4-fluoro-2-methylpheny])(((S)- tetrahydrofuran-2-yl)methyl)amino)cyclohexyl) (methyl)amino)-5-methyl-6-oxo-5,6-dihydro-1,5- naphthyridine-2,7-dicarbonitrile |
| 169 | 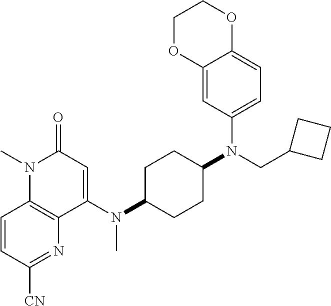 | cis-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2- carbonitrile |
| 170 | 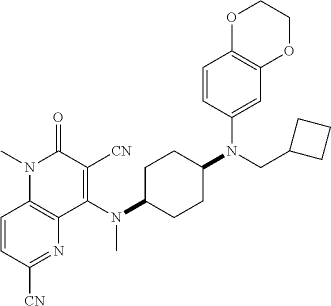 | cis-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2,7- dicarbonitrile |
| 171 | 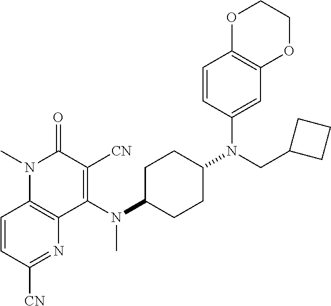 | trans-8-((4-((cyclobutylmethyl)(2,3-dihydrobenzo[b] [1,4]dioxin-6-yl)amino)cyclohexyl)(methyl)amino)- 5-methyl-6-oxo-5,6-dihydro-1,5-naphthyridine-2,7- dicarbonitrile |
| 172 | 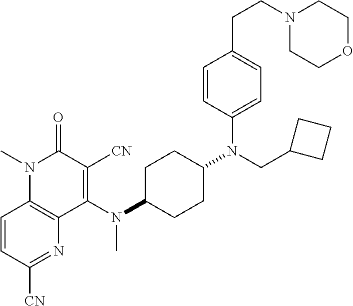 | trans-8-((4-((cyclobutylmethyl)(4-(2- morpholinoethyl)phenyl)amino)cyclohexyl)(methy]) amino)-5-methyl-6-oxo-5,6-dihydro-1,5- naphthyridine-2,7-dicarbonitrile |
| 173 | 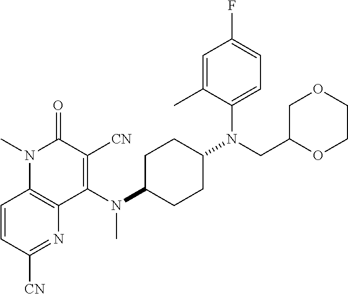 | trans-8-((4-(((1,4-dioxan-2-yl)methyl)(4-fluoro-2- methylphenyl)amino)cyclohexyl)(methyl)amino)-5- methyl-6-oxo-5,6-dihydro-1,5-naphthyridine-2,7- dicarbonitrile |
| 174 | 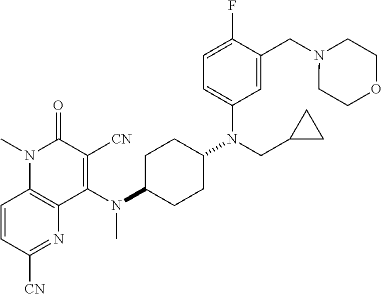 | trans-8-((-4-((cyclopropylmethyl)(4-fluoro-3- (morpholinomethyl)phenyl)amino)cyclohexyl) (methyl)amino)-5-methyl-6-oxo-5,6-dihydro-1,5- naphthyridine-2,7-dicarbonitrile |
| 175 | 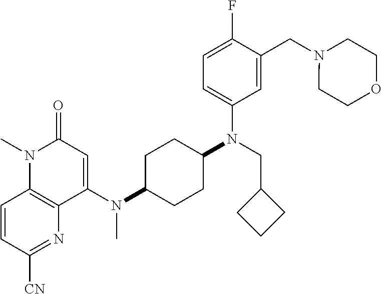 | cis-8-((-4-((cyclobutylmethyl)(4-fluoro-3- (morpholinomethyl)phenyl)amino)cyclohexyl) (methyl)amino)-5-methyl-6-oxo-5,6-dihydro-1,5- naphthyridine-2-carbonitrile |
| 176 | 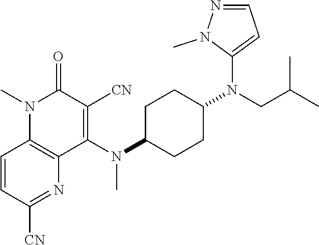 | trans-8-((-4-(isobutyl(1-methyl-1H-pyrazol-5-yl) amino)cyclohexyl)(methyl)amino)-5-methyl-6-oxo- 5,6-dihydro-1,5-naphthyridine-2,7-dicarbonitrile |
| 177 | 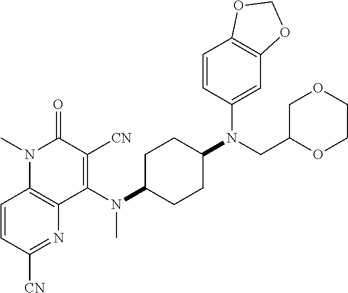 | cis-8-((4-(((1,4-dioxan-2-yl)methyl)(benzo[d][1,3] dioxol-5-yl)amino)cyclohexyl)(methyl)amino)-5- methyl-6-oxo-5,6-dihydro-1,5-naphthyridine-2,7- dicarbonitrile |
| 178 | 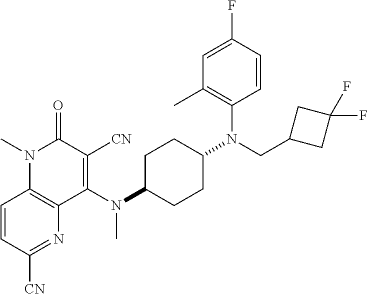 | trans-8-((-4-(((3,3-difluorocyclobutyl)methyl)(4- fluoro-2-methylphenyl)amino)cyclohexyl)(methy]) amino)-5-methyl-6-oxo-5,6-dihydro-1,5- naphthyridine-2,7-dicarbonitrile |
| 179 | 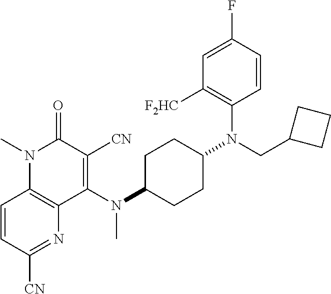 | trans-8-((-4-((cyclobutylmethyl)(2-(difluoromethyl)- 4-fluorophenyl)amino)cyclohexyl)(methyl)amino)-5- methyl-6-oxo-5,6-dihydro-1,5-naphthyridine-2,7- dicarbonitrile |
| 180 | 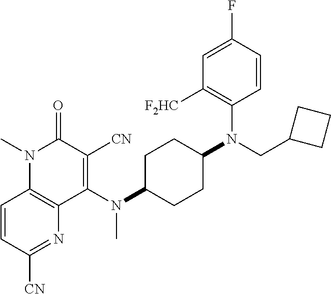 | cis-8-((-4-((cyclobutylmethyl)(2-(difluoromethyl)-4- fluorophenyl)amino)cyclohexyl)(methyl)amino)-5- methyl-6-oxo-5,6-dihydro-1,5-naphthyridine-2,7- dicarbonitrile |
| 181 | 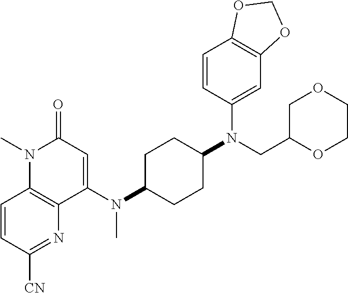 | cis-8-((4-(((1,4-dioxan-2-yl)methyl)(benzo[d][1,3] dioxol-5-yl)amino)cyclohexyl)(methyl)amino)-5- methyl-6-oxo-5,6-dihydro-1,5-naphthyridine-2- carbonitrile |
| 182 | 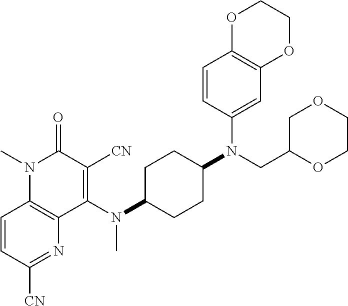 | cis-8-((4-(((1,4-dioxan-2-yl)methyl)(2,3- dihydrobenzo[b][1,4]dioxin-6-yl)amino)cyclohexyl) (methyl)amino)-5-methyl-6-oxo-5,6-dihydro-1,5- naphthyridine-2,7-dicarbonitrile |
| 183 | 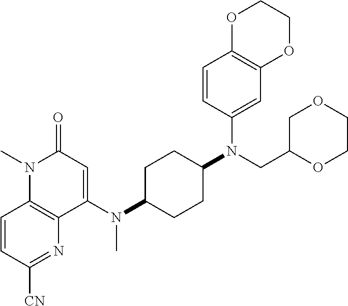 | cis-8-((4-(((1,4-dioxan-2-yl)methyl)(2,3- dihydrobenzo[b][1,4]dioxin-6-yl)amino)cyclohexyl) (methyl)amino)-5-methyl-6-oxo-5,6-dihydro-1,5- naphthyridine-2-carbonitrile |
| 184 | 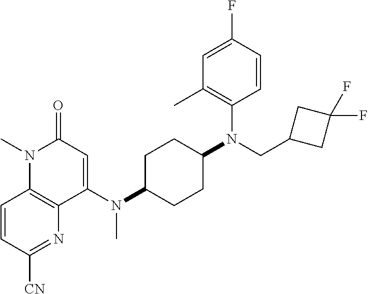 | cis-8-((-4-(((3,3-difluorocyclobutyl)methyl)(4-fluoro- 2-methylphenyl)amino)cyclohexyl)(methyl)amino)-5- methyl-6-oxo-5,6-dihydro-1,5-naphthyridine-2- carbonitrile |
| 185 | 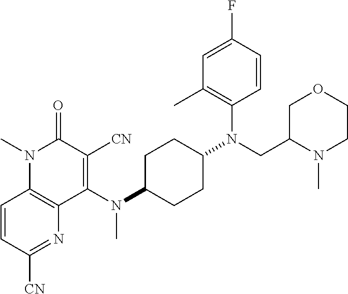 | trans-8-((-4-((4-fluoro-2-methylpheny])((4- methylmorpholin-3-yl)methyl)amino)cyclohexyl) (methyl)amino)-5-methyl-6-oxo-5,6-dihydro-1,5- naphthyridine-2,7-dicarbonitrile |
| 186 | 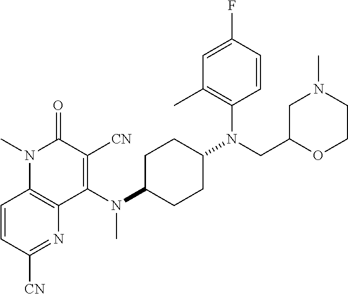 | trans-8-((-4-((4-fluoro-2-methylpheny])((4- methylmorpholinium-2-yl)methyl)amino)cyclohexyl) (methyl)amino)-5-methyl-6-oxo-5,6-dihydro-1,5- naphthyridine-2,7-dicarbonitriletrifluoroacetate |
| 187 | 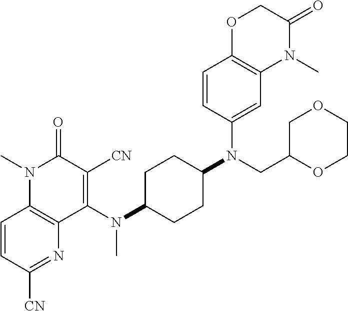 | cis-8-((4-(((1,4-dioxan-2-yl)methyl)(4-methyl-3-oxo- 3,4-dihydro-2H-benzo[b][1,4]oxazin-6-yl)amino) cyclohexyl)(methyl)amino)-5-methyl-6-oxo-5,6- dihydro-1,5-naphthyridine-2,7-dicarbonitrile |
| 188 | 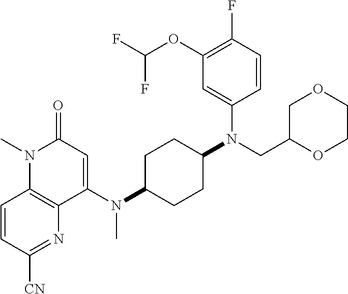 | cis-8-((-4-(((1,4-dioxan-2-yl)methyl)(3- (difluoromethoxy)-4-fluorophenyl)amino)cyclohexyl) (methyl)amino)-5-methyl-6-oxo-5,6-dihydro-1,5- naphthyridine-2-carbonitrile |
| 189 | 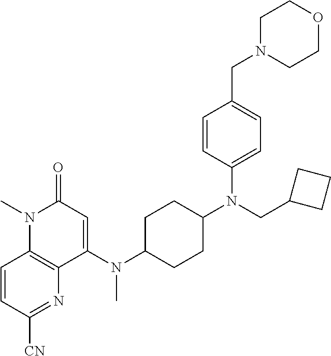 [SMILES: CN(c1cc(=O)n(C)c2ccc(C#N)nc12)C1CCC(N(CC2CCC2)c2ccc(CN3CCOCC3)cc2)CC1] | (cisortrans)-8-((4-((cyclobutylmethyl)(4- (morpholinomethyl)phenyl)amino)cyclohexyl) (methyl)amino)-5-methyl-6-oxo-5,6-dihydro-1,5- naphthyridine-2-carbonitrile |
| 190 | 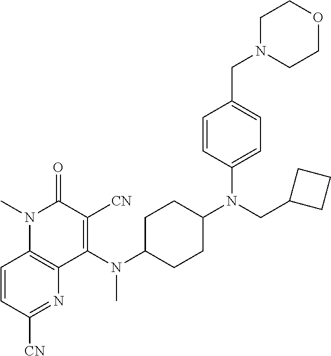 [SMILES: CN(c1c(C#N)c(=O)n(C)c2ccc(C#N)nc12)C1CCC(N(CC2CCC2)c2ccc(CN3CCOCC3)cc2)CC1] | (cisortrans)-8-((4-((cyclobutylmethyl)(4- (morpholinomethyl)phenyl)amino)cyclohexyl) (methyl)amino)-5-methyl-6-oxo-5,6-dihydro-1,5- naphthyridine-2,7-dicarbonitrile |
| 191 | 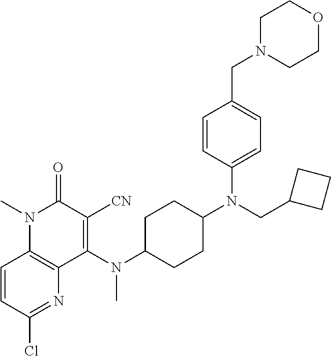 [SMILES: CN(c1c(C#N)c(=O)n(C)c2ccc(Cl)nc12)C1CCC(N(CC2CCC2)c2ccc(CN3CCOCC3)cc2)CC1] | (cisortrans)-6-chloro-4-((4-((cyclobutylmethyl)(4- (morpholinomethyl)phenyl)amino)cyclohexyl) (methyl)amino)-1-methyl-2-oxo-1,2-dihydro-1,5- naphthyridine-3-carbonitrile |
| 192 | 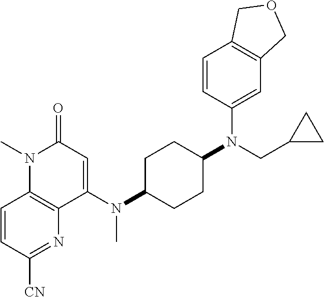 | cis-8-((4-((cyclopropylmethyl)(1,3- (methyl)amino)-5-methyl-6-oxo-5,6-dihydro-1,5- dihydroisobenzofuran-5-yl)amino)cyclohexyl) naphthyridine-2-carbonitrile |
| 193 | 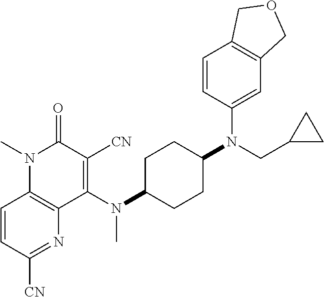 | cis-8-((4-((cyclopropylmethyl)(1,3- dihydroisobenzofuran-5-yl)amino)cyclohexyl) (methyl)amino)-5-methyl-6-oxo-5,6-dihydro-1,5- naphthyridine-2,7-dicarbonitrile |
| 194 | 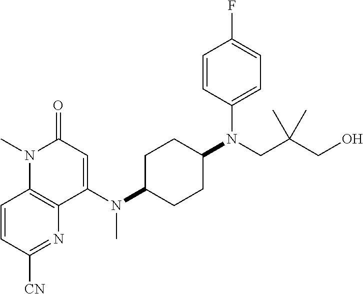 | cis-8-((-4-((4-fluorophenyl)(3-hydroxy-2,2- dimethylpropyl)amino)cyclohexyl)(methyl)amino)-5- methyl-6-oxo-5,6-dihydro-1,5-naphthyridine-2- carbonitrile |
| 195 | 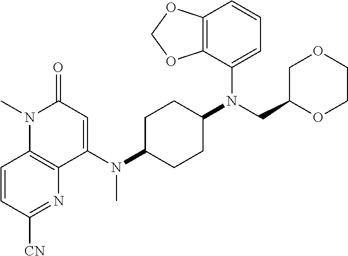 | cis-8-((-4-((((SorR)-1,4-dioxan-2-yl)methyl) (benzo[d][1,3]dioxol-4-yl)amino)cyclohexyl)(methyl) amino)-5-methyl-6-oxo-5,6-dihydro-1,5- naphthyridine-2-carbonitrile |
| 196 | 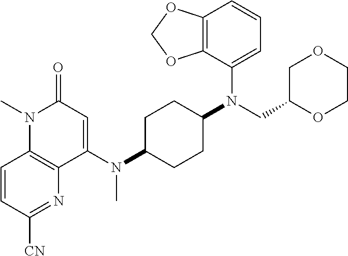 | cis-8-((-4-((((RorS)-1,4-dioxan-2-yl)methyl) (benzo[d][1,3]dioxol-4-yl)amino)cyclohexyl)(methyl) amino)-5-methyl-6-oxo-5,6-dihydro-1,5- naphthyridine-2-carbonitrile |
| 197 | 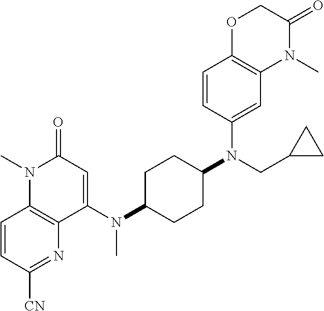 | cis-8-((4-((cyclopropylmethyl)(4-methyl-3-oxo-3,4- dihydro-2H-benzo[b][1,4]oxazin-6-yl)amino) cyclohexyl)(methyl)amino)-5-methyl-6-oxo-5,6- dihydro-1,5-naphthyridine-2-carbonitrile |
| 198 | 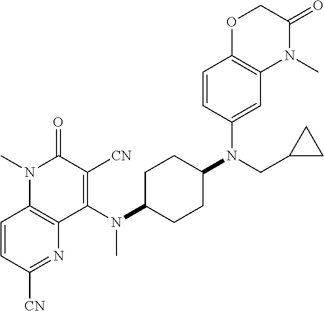 | cis-8-((4-((cyclopropylmethyl)(4-methyl-3-oxo-3,4- dihydro-2H-benzo[b][1,4]oxazin-6-yl)amino) cyclohexyl)(methyl)amino)-5-methyl-6-oxo-5,6- dihydro-1,5-naphthyridine-2,7-dicarbonitrile |
| 199 | 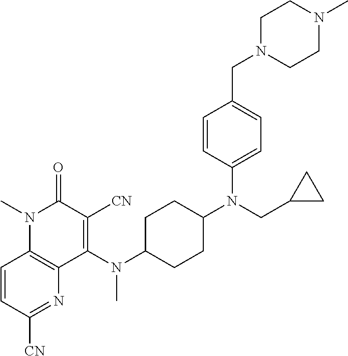 [SMILES: CN1CCN(Cc2ccc(N(CC3CC3)C3CCC(N(C)c4c(C#N)c(=O)n(C)c5ccc(C#N)nc45)CC3)cc2)CC1] | (cis or trans)-8-((4-((cyclopropylmethyl)(4-((4- methylpiperazin-1-yl)methyl)phenyl)amino) cyclohexyl)(methyl)amino)-5-methyl-6-oxo-5,6- dihydro-1,5-naphthyridine-2,7-dicarbonitrile |
| 200 | 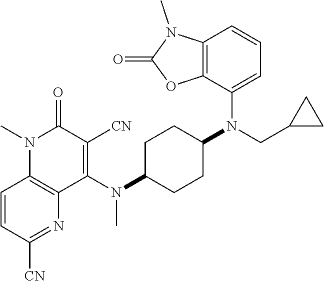 | cis-8-((-4-((cyclopropylmethyl)(3-methyl-2-oxo-2,3- dihydrobenzo[d]oxazol-7-yl)amino)cyclohexyl) (methyl)amino)-5-methyl-6-oxo-5,6-dihydro-1,5- naphthyridine-2,7-dicarbonitrile |
| 201 | 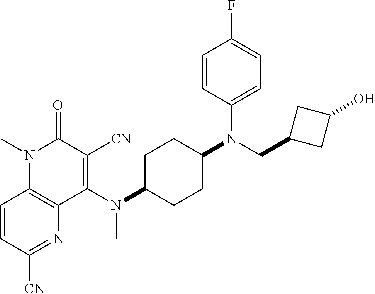 | cis-8-((-4-((4-fluorophenyl)((trans-3- hydroxycyclobutyl)methyl)amino)cyclohexyl) (methyl)amino)-5-methyl-6-oxo-5,6-dihydro-1,5- naphthyridine-2,7-dicarbonitrile |
| 202 | 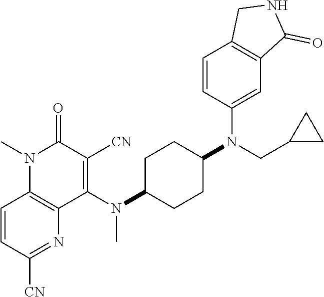 | cis-8-((-4-((cyclopropylmethyl)(3-oxoisoindolin-5-yl) amino)cyclohexyl)(methyl)amino)-5-methyl-6-oxo- 5,6-dihydro-1,5-naphthyridine-2,7-dicarbonitrile |
| 203 | 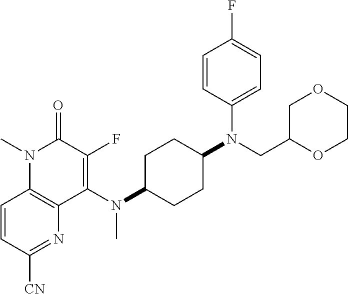 | cis-8-((-4-(((1,4-dioxan-2-yl)methyl)(4-fluorophenyl) amino)cyclohexyl)(methyl)amino)-7-fluoro-5- methyl-6-oxo-5,6-dihydro-1,5-naphthyridine-2- carbonitrile |
| 204 | 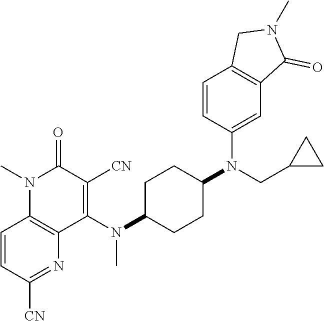 | cis-8-((-4-((cyclopropylmethyl)(2-methyl-3- oxoisoindolin-5-yl)amino)cyclohexyl)(methyl) amino)-5-methyl-6-oxo-5,6-dihydro-1,5- naphthyridine-2,7-dicarbonitrile |
| 205 | 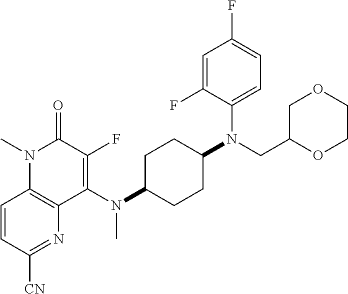 | cis-8-((-4-(((1,4-dioxan-2-yl)methyl)(2,4- difluorophenyl)amino)cyclohexyl)(methyl)amino)-7- fluoro-5-methyl-6-oxo-5,6-dihydro-1,5- naphthyridine-2-carbonitrile |
| 206 | 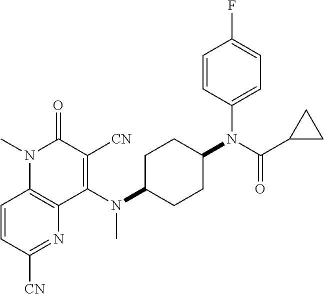 | cis-N-(4-((3,6-dicyano-1-methyl-2-oxo-1,2-dihydro- 1,5-naphthyridin-4-yl)(methyl)amino)cyclohexyl)-N- (4-fluorophenyl)cyclopropanecarboxamide |
| 207 | 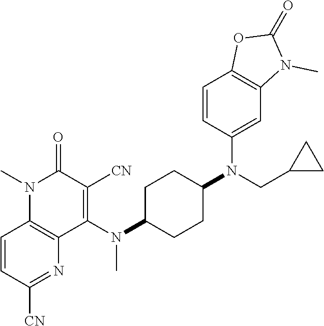 | cis-8-((-4-((cyclopropylmethyl)(3-methyl-2-oxo-2,3- dihydrobenzo[d]oxazol-5-yl)amino)cyclohexyl) (methyl)amino)-5-methyl-6-oxo-5,6-dihydro-1,5- naphthyridine-2,7-dicarbonitrile |
| 208 | 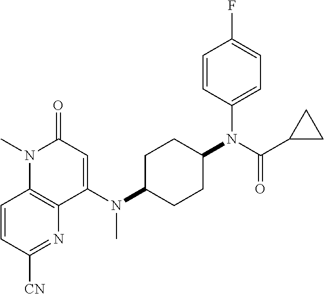 | cis-N-(4-((6-cyano-1-methyl-2-oxo-1,2-dihydro-1,5- naphthyridin-4-yl)(methyl)amino)cyclohexyl)-N-(4- fluorophenyl)cyclopropanecarboxamide |
| 209 | 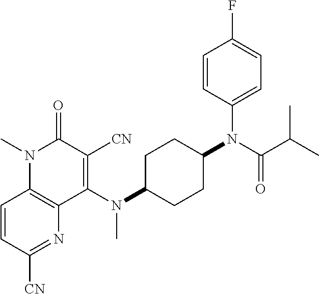 | cis-N-(4-((3,6-dicyano-1-methyl-2-oxo-1,2-dihydro- 1,5-naphthyridin-4-yl)(methyl)amino)cyclohexyl)-N- (4-fluorophenyl)isobutyramide |
| 210 | 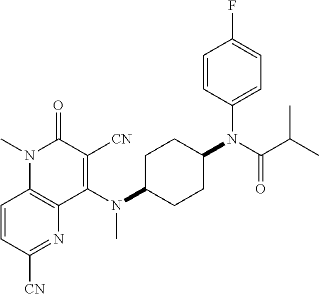 | cis-N-(4-((3,6-dicyano-1-methyl-2-oxo-1,2-dihydro- 1,5-naphthyridin-4-yl)(methyl)amino)cyclohexyl)-N- (4-fluorophenyl)isobutyramide |
| 211 | 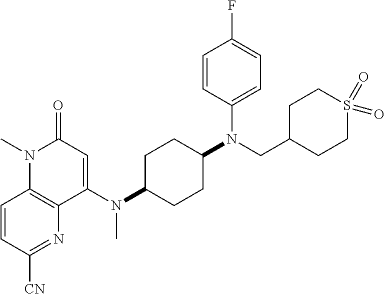 | cis-8-((4-(((1,1-dioxidotetrahydro-2H-thiopyran-4-yl) methyl)(4-fluorophenyl)amino)cyclohexyl)(methyl) amino)-5-methyl-6-oxo-5,6-dihydro-1,5- naphthyridine-2-carbonitrile |
| 212 | 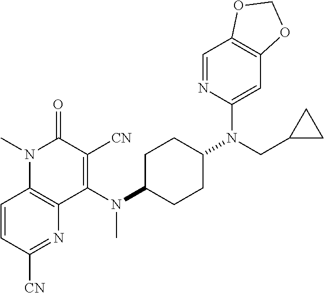 | trans-8-((-4-([1,3]dioxolo[4,5-c]pyridin-6- yl(cyclopropylmethyl)amino)cyclohexyl)(methyl) amino)-5-methyl-6-oxo-5,6-dihydro-1,5- naphthyridine-2,7-dicarbonitrile |
| 213 | 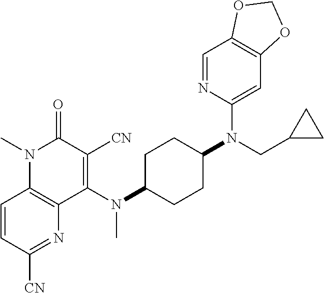 | cis-8-((-4-([1,3]dioxolo[4,5-c]pyridin-6- yl(cyclopropylmethyl)amino)cyclohexyl)(methyl) amino)-5-methyl-6-oxo-5,6-dihydro-1,5- naphthyridine-2,7-dicarbonitrile |
| 214 | 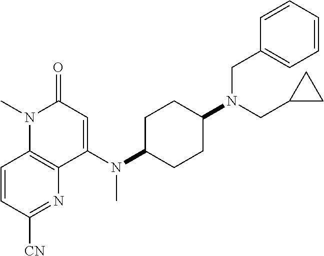 | cis-8-((-4-(benzyl(cyclopropylmethyl)amino) cyclohexyl)(methyl)amino)-5-methyl-6-oxo-5,6- dihydro-1,5-naphthyridine-2-carbonitrile |

or a pharmaceutically acceptable salt, solvate, hydrate, isomer, or isotope thereof.

40. A pharmaceutical composition comprising a compound claim 1 or 39, or a pharmaceutically acceptable salt, solvate, hydrate, isomer, or isotope thereof, and at least one pharmaceutically acceptable excipient.

41. A method for inhibiting the activity of at least one diacylglycerol kinase, comprising contacting the diacylglycerol kinase with a compound of claim 1 or 39, or a pharmaceutically acceptable salt, solvate, hydrate, isomer, or isotope thereof, or with the pharmaceutical composition of claim 40.

42. The method of claim 41, wherein the diacylglycerol kinase is a diacylglycerol kinase alpha (DGKα) or a diacylglycerol kinase zeta (DGKζ).

43. A method of treating a disease or disorder associated with the activity of DGKα, DGKζ, or both DGKα and DGKζ, comprising administering to a subject in need thereof an effective amount of a compound or claim 1 or 39, or a pharmaceutically acceptable salt, solvate, hydrate, isomer, or isotope thereof, or an effective amount of the pharmaceutical composition of claim 40.

44. A method of treating a proliferative disorder or a viral infection, comprising administering to a subject in need thereof an effective amount of a compound or claim 1 or 39, or a pharmaceutically acceptable salt, solvate, hydrate, isomer, or isotope thereof, or an effective amount of the pharmaceutical composition of claim 40.

45. The method of claim 44, wherein the proliferative disorder is cancer.

46. The method of claim 45, wherein the cancer is cancer of the colon, pancreatic cancer, breast cancer, prostate cancer, lung cancer, ovarian cancer, cervical cancer, renal cancer, cancer of the head and neck, lymphoma, leukemia, or melanoma.

47-52. (canceled)
